(19) United States
(12) Patent Application Publication
(10) Pub. No.: US 2024/0300978 A1
(43) Pub. Date: Sep. 12, 2024

(54) Novel Compound and Organic Electroluminescence Device Using the Same

(71) Applicant: IDEMITSU KOSAN CO., LTD., Tokyo (JP)

(72) Inventors: Yoshinao SHIRASAKI, Sodegaura-shi (JP); Masatoshi SAITO, Sodegaura-shi (JP); Kei YOSHIDA, Sodegaura-shi (JP); Tetsuya MASUDA, Sodegaura-shi (JP); Masato NAKAMURA, Sodegaura-shi (JP); Tomokatsu KUSHIDA, Sodegaura-shi (JP)

(73) Assignee: IDEMITSU KOSAN CO., LTD., Tokyo (JP)

(21) Appl. No.: 18/361,304

(22) Filed: Jul. 28, 2023

Related U.S. Application Data
(63) Continuation of application No. 17/299,937, filed on Jun. 4, 2021.

(30) Foreign Application Priority Data
Dec. 7, 2018 (JP) 2018-229962
Jun. 7, 2019 (JP) 2019-107425

(51) Int. Cl. C07D 519/00; H10K 50/11; H10K 50/16; H10K 85/60; H10K 101/10

(52) U.S. Cl. CPC C07D 519/00; H10K 85/624; H10K 85/654; H10K 85/6572; H10K 85/6574; H10K 85/6576; H10K 50/11; H10K 50/166; H10K 2101/10

(57) ABSTRACT

A compound represented by the following formula (A1) (X₁ is O or S; two or more of Y₁, Y₂, and Y₃ are N; and Ar₁ is an aryl group including 6 to 50 ring carbon atoms, comprising a benzene ring having at least substituent Ar₂ at the ortho-position; Ar₂ is an aryl group; and Ar₃ is a predetermined group).

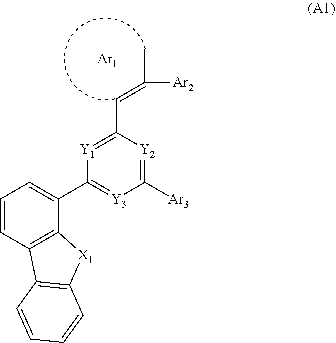

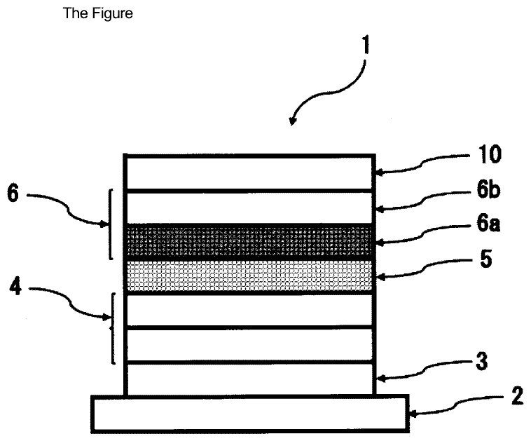

### CROSS-REFERENCE TO RELATED APPLICATIONS

The present application is a continuation of U.S. application Ser. No. 17/299,937, filed on Jun. 4, 2021, which claims priority under 35 U.S.C. § 371 to International Patent Application No. PCT/JP2019/047833, filed Dec. 6, 2019, which claims priority to and the benefit of Japanese Patent Application Nos. 2018-229962, filed on Dec. 7, 2018, and 2019-107425, filed on Jun. 7, 2019. The contents of these applications are hereby incorporated by reference in their entireties.

### TECHNICAL FIELD

The invention relates to a novel compound and an organic electroluminescence device using the same.

### BACKGROUND ART

When voltage is applied to an organic electroluminescence device (hereinafter, referred to as an organic EL device in several cases), holes and electrons are injected into an emitting layer from an anode and a cathode, respectively. Then, thus injected holes and electrons are recombined in the emitting layer, and excitons are formed therein.

Patent Documents 1 and 2 disclose a compound in which an azine ring and a dibenzothiophene ring are bonded with or without a linking group, as a material for an organic EL device, and an organic EL device using the same.

### RELATED ART DOCUMENT

### Patent Document

[Patent Document 1] WO2007/069569
[Patent Document 2] WO2013/077352

### SUMMARY OF THE INVENTION

It is an object of the invention to provide a novel compound which can be used as a material for an organic electroluminescence device, that makes the device to have high luminous efficiency, as well as an organic electroluminescence device which exhibits high luminous efficiency using the same.

According to the invention, the following novel compound, an electron transporting material for an organic electroluminescence device, an organic electroluminescence device, and an electronic apparatus are provided.
1. A compound represented by the following formula (A1):

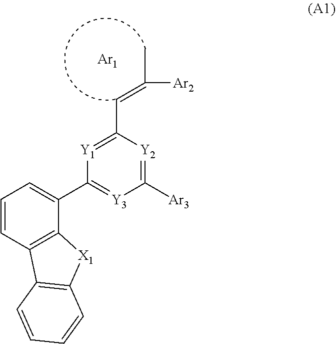

wherein in the formula (A1),
X₁ is O or S;
Y₁, Y₂, and Y₃ are independently CH or N;
provided that two or more of Y₁, Y₂, and Y₃ are N;
Ar₁ is an aryl group including 6 to 50 ring carbon atoms, comprising a benzene ring having at least substituent Ar₂ at the ortho-position;
Ar₂ is a substituted or unsubstituted aryl group including 6 to 50 ring carbon atoms;
Ar₁ and Ar₂ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring;
Ar₃ is a group selected from the group consisting of a group represented by the following formula (A2-1), a group represented by the following formula (A2-2), a group represented by the following formula (A2-3), and a group represented by the following formula (A2-4):

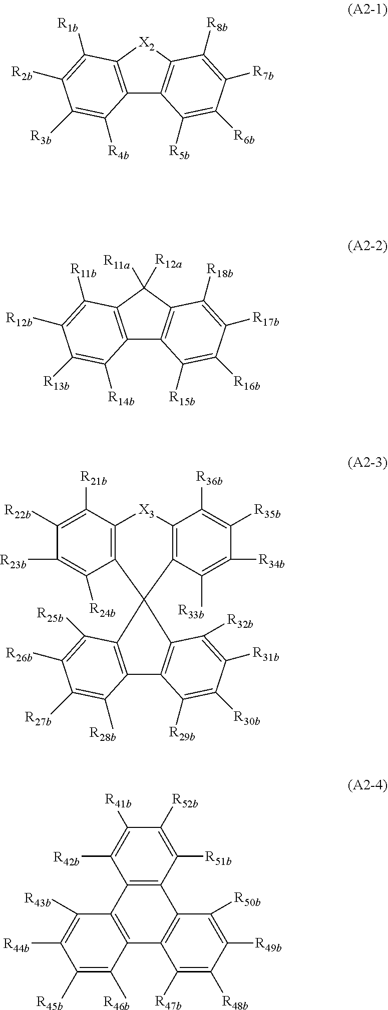

wherein in the formula (A2-1),
X₂ is O or S;
one of R_{1b} to R_{8b} is a single bond bonding with the carbon atom between Y₂ and Y₃, and the others are hydrogen atoms;
wherein in the formula (A2-2),
R₁₁ₐ and R₁₂ₐ form a substituted or unsubstituted, saturated or unsaturated ring by bonding with each other, or do not form a substituted or unsubstituted, saturated or unsaturated ring;
one of R_{11b} to R_{18b} is a single bond bonding with the carbon atom between Y₂ and Y₃;
R₁₁ₐ and R₁₂ₐ which do not form the substituted or unsubstituted, saturated or unsaturated ring, and R_{11b} to R_{18b} which are not the single bond are independently
a hydrogen atom,
a substituted or unsubstituted alkyl group including 1 to 50 carbon atoms, or
a substituted or unsubstituted aryl group including 6 to 50 ring carbon atoms;
   wherein in the formula (A2-3),
   one of R_{21b} to R_{36b} is a single bond bonding with the carbon atom between Y₂ and Y₃;
   X₃ is NR₂₁ₐ, CR₂₂ₐR₂₃ₐ, O, or S;
   R₂₁ₐ form a substituted or unsubstituted, saturated or unsaturated ring by bonding either or both of R_{21b} which is not a single bond or R_{36b} which is not a single bond, or does not form the substituted or unsubstituted, saturated or unsaturated ring;
   R_{22b} to R_{35b} which are not single bonds, R_{21b} and R_{36b} which are not single bonds and which do not form the substituted or unsubstituted, saturated or unsaturated ring, R₂₁ₐ which does not form the substituted or unsubstituted, saturated or unsaturated ring, and R₂₂ₐ and R₂₃ₐ are independently
a hydrogen atom,
a substituted or unsubstituted alkyl group including 1 to 50 carbon atoms, or
a substituted or unsubstituted aryl group including 6 to 50 ring carbon atoms;
   wherein in the formula (A2-4),
   one of R_{41b} to R_{52b} is a single bond bonding with the carbon atom between Y₂ and Y₃; and
   R_{41b} to R_{52b} which are not the single bond are independently
a hydrogen atom,
a substituted or unsubstituted alkyl group including 1 to 50 carbon atoms, or
a substituted or unsubstituted aryl group including 6 to 50 ring carbon atoms.
2. An electron-transporting material for an organic electroluminescence device, comprising the compound represented by the formula (A1).
3. An organic electroluminescence device comprising an anode, an organic layer, and a cathode in this order, wherein
   the organic layer comprises the compound represented by the formula (A1).
4. An organic electroluminescence device comprising an anode, an emitting layer, an electron-transporting region, and a cathode in this order, wherein
   the electron transporting region comprises the compound represented by the formula (A1).
5. An electronic apparatus comprising the above-mentioned organic electroluminescence device.

According to the invention, a novel compound which can be used as a material for an organic electroluminescence device, that makes the device to have high luminous efficiency, as well as an organic electroluminescence device which exhibits high luminous efficiency using the same can be provided.

### BRIEF DESCRIPTION OF THE DRAWINGS

the FIGURE is a schematic diagram of the organic EL device according to an aspect of the invention.

### MODE FOR CARRYING OUT THE INVENTION

In this specification, a hydrogen atom means an atom including isotopes different in the number of neutrons, namely, a protium, a deuterium and a tritium.

In this specification, to a bondable position in which a symbol such as “R”, or “D” representing a deuterium atom is not specified in a chemical formula, a hydrogen atom, that is, a protium atom, a deuterium atom, or a tritium atom is bonded thereto.

In this specification, a term “ring carbon atoms” represents the number of carbon atoms among atoms forming a subject ring itself of a compound having a structure in which atoms are bonded in a ring form (for example, a monocyclic compound, a fused ring compound, a cross-linked compound, a carbocyclic compound or a heterocyclic compound). When the subject ring is substituted by a substituent, the carbon contained in the substituent is not included in the number of ring carbon atoms. The same shall apply to the “ring carbon atoms” described below, unless otherwise noted. For example, a benzene ring has 6 ring carbon atoms, a naphthalene ring has 10 ring carbon atoms, a pyridine ring has 5 ring carbon atoms, and a furan ring has 4 ring carbon atoms. Further, for example, a 9,9-diphenylfluorenyl group has 13 ring carbon atoms, and a 9,9′-spirobifluorenyl group has 25 ring carbon atoms.

Further, when the benzene ring or the naphthalene ring is substituted by an alkyl group as a substituent, for example, the number of carbon atoms of the alkyl group is not included in the ring carbon atoms.

In this specification, a term “ring atoms” represents the number of atoms forming a subject ring itself of a compound having a structure in which atoms are bonded in a ring form (for example, a monocycle, a fused ring and a ring assembly) (for example, a monocyclic compound, a fused ring compound, a cross-linked compound, a carbocyclic compound or a heterocyclic compound). The term “ring atoms” does not include atoms which do not form the ring (for example, a hydrogen atom which terminates a bond of the atoms forming the ring) or atoms contained in a substituent when the ring is substituted by the substituent. The same shall apply to the “ring atoms” described below, unless otherwise noted. For example, a pyridine ring has 6 ring atoms, a quinazoline ring has 10 ring atoms, and a furan ring has 5 ring atoms. A hydrogen atom bonded with a carbon atom of the pyridine ring or the quinazoline ring or an atom forming the substituent is not included in the number of the ring atoms.

In this specification, a term “XX to YY carbon atoms” in an expression of “substituted or unsubstituted ZZ group including XX to YY carbon atoms” represents the number of carbon atoms when the ZZ group is unsubstituted. The number of carbon atoms of a substituent when the ZZ group is substituted is not included. Here, “YY” is larger than “XX”, and “XX” and “YY” each mean an integer of 1 or more.

In this specification, a term “XX to YY atoms” in an expression of “substituted or unsubstituted ZZ group including XX to YY atoms” represents the number of atoms when the ZZ group is unsubstituted. The number of atoms of a substituent when the group is substituted is not included. Here, “YY” is larger than “XX”, and “XX” and “YY” each mean an integer of 1 or more.

A term “unsubstituted” in the case of “substituted or unsubstituted ZZ group” means that the ZZ group is not substituted by a substituent, and a hydrogen atom is bonded therewith. Alternatively, a term “substituted” in the case of “substituted or unsubstituted ZZ group” means that one or more hydrogen atoms in the ZZ group are substituted by a substituent. Similarly, a term “substituted” in the case of “BB group substituted by an AA group” means that one or more hydrogen atoms in the BB group are substituted by the AA group.

Hereinafter, the substituent described in this specification will be described.

The number of the ring carbon atoms of the “unsubstituted aryl group” described in this specification is 6 to 50, preferably 6 to 30, and more preferably 6 to 18, unless otherwise specified.

The number of the ring carbon atoms of the “unsubstituted heterocyclic group” described in this specification is 5 to 50, preferably 5 to 30, and more preferably 5 to 18, unless otherwise specified.

The number of the carbon atoms of the “unsubstituted alkyl group” described in this specification is 1 to 50, preferably 1 to 20, and more preferably 1 to 6, unless otherwise specified.

The number of the carbon atoms of the “unsubstituted alkenyl group” described in this specification is 2 to 50, preferably 2 to 20, and more preferably 2 to 6, unless otherwise specified.

The number of the carbon atoms of the “unsubstituted alkynyl group” described in this specification is 2 to 50, preferably 2 to 20, and more preferably 2 to 6, unless otherwise specified.

The number of the ring carbon atoms of the “unsubstituted cycloalkyl group” described in this specification is 3 to 50, preferably 3 to 20, and more preferably 3 to 6, unless otherwise specified.

The number of the ring carbon atoms of the “unsubstituted arylene group” described in this specification is 6 to 50, preferably 6 to 30, and more preferably 6 to 18, unless otherwise specified.

The number of the ring atoms of the “unsubstituted divalent heterocyclic group” described in this specification is 5 to 50, preferably 5 to 30, and more preferably 5 to 18, unless otherwise specified.

The number of the carbon atoms of the “unsubstituted alkylene group” described in this specification is 1 to 50, preferably 1 to 20, and more preferably 1 to 6, unless otherwise specified.

Specific examples (specific example group G1) of the “substituted or unsubstituted aryl group” described in this specification include an unsubstituted aryl group and a substituted aryl group described below. (Here, a term “unsubstituted aryl group” refers to a case where the “substituted or unsubstituted aryl group” is the “unsubstituted aryl group,” and a term “substituted aryl group” refers to a case where the “substituted or unsubstituted aryl group” is the “substituted aryl group”. Hereinafter, a case of merely “aryl group” includes both the “unsubstituted aryl group” and the “substituted aryl group”.

The “substituted aryl group” refers to a case where the “unsubstituted aryl group” has a substituent, and specific examples thereof include a group in which the “unsubstituted aryl group” has the substituent, and a substituted aryl group described below. It should be noted that examples of the “unsubstituted aryl group” and examples of the “substituted aryl group” listed in this specification are only one example, and the “substituted aryl group” described in this specification also includes a group in which a group in which “unsubstituted aryl group” has a substituent further has a substituent, and a group in which “substituted aryl group” further has a substituent, and the like.

An unsubstituted aryl group:
a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group, and
a perylenyl group.

A substituted aryl group:
an o-tolyl group,
a m-tolyl group,
a p-tolyl group,
a p-xylyl group,
a m-xylyl group,
an o-xylyl group,
a p-isopropyl phenyl group,
a m-isopropyl phenyl group,
an o-isopropyl phenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group
a 9,9-di(4-methylphenyl)fluorenyl group,
a 9,9-di(4-isopropylphenyl)fluorenyl group,
a 9,9-di(4-t-butylphenyl)fluorenyl group,
a cyanophenyl group,
a triphenylsilylphenyl group,
a trimethylsilylphenyl group,
a phenylnaphthyl group, and
a naphthylphenyl group.

The “heterocyclic group” described in this specification is a ring group including at least one hetero atom in the ring atom. Specific examples of the hetero atom include a nitrogen atom, an oxygen atom, a sulfur atom, a silicon atom, a phosphorus atom and a boron atom.

The “heterocyclic group” described in this specification may be a monocyclic group, or a fused ring group.

The “heterocyclic group” described in this specification may be an aromatic heterocyclic group, or an aliphatic heterocyclic group.

Specific examples (specific example group G2) of the “substituted or unsubstituted heterocyclic group” include an unsubstituted heterocyclic group and a substituted heterocyclic group described below. (Here, the unsubstituted heterocyclic group refers to a case where the “substituted or unsubstituted heterocyclic group” is the “unsubstituted heterocyclic group,” and the substituted heterocyclic group refers to a case where the “substituted or unsubstituted heterocyclic group” is the “substituted heterocyclic group”. Hereinafter, the case of merely “heterocyclic group” includes both the “unsubstituted heterocyclic group” and the “substituted heterocyclic group”.

The “substituted heterocyclic group” refers to a case where the “unsubstituted heterocyclic group” has a substituent, and specific examples thereof include a group in which the “unsubstituted heterocyclic group” has a substituent, and a substituted heterocyclic group described below. It should be noted that examples of the “unsubstituted heterocyclic group” and examples of the “substituted heterocyclic group” listed in this specification are merely one example, and the “substituted heterocyclic group” described in this specification also includes a group in which “unsubstituted heterocyclic group” which has a substituent further has a substituent, and a group in which “substituted heterocyclic group” further has a substituent, and the like.

An unsubstituted heterocyclic group including a nitrogen atom:
a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

An unsubstituted heterocyclic group including an oxygen atom:
a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

An unsubstituted heterocyclic group including a sulfur atom:
a thienyl group,
a thiazolyl group,
an isothiazolyl group,
a thiadiazolyl group,
a benzothiophenyl group,
an isobenzothiophenyl group,
a dibenzothiophenyl group,
a naphthobenzothiophenyl group,
a benzothiazolyl group,
a benzisothiazolyl group,
a phenothiazinyl group,
a dinaphthothiophenyl group,
an azadibenzothiophenyl group,
a diazadibenzothiophenyl group,
an azanaphthobenzothiophenyl group, and
a diazanaphthobenzothiophenyl group.

A substituted heterocyclic group including a nitrogen atom:
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

A substituted heterocyclic group including an oxygen atom:
a phenyldibenzofuranyl group,
a methyldibenzofuranyl group,
a t-butyldibenzofuranyl group, and
a monovalent residue of spiro[9H-xanthene-9,9′-[9H]fluorene].

A substituted heterocyclic group including a sulfur atom:
a phenyldibenzothiophenyl group,
a methyldibenzothiophenyl group,
a t-butyldibenzothiophenyl group, and
a monovalent residue of spiro[9H-thioxantene-9,9′-[9H]fluorene].

A monovalent group derived from the following unsubstituted heterocyclic ring containing at least one of a nitrogen atom, an oxygen atom and a sulfur atom by removal of one hydrogen atom bonded to the ring atoms thereof, and a monovalent group in which a monovalent group derived from the following unsubstituted heterocyclic ring has a substituent by removal of one hydrogen atom bonded to the ring atoms thereof:

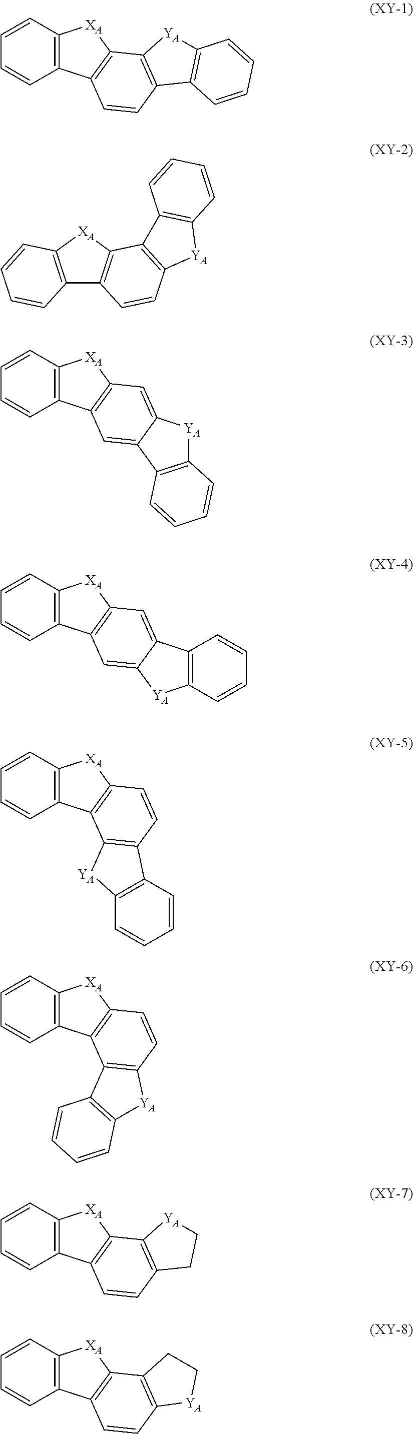

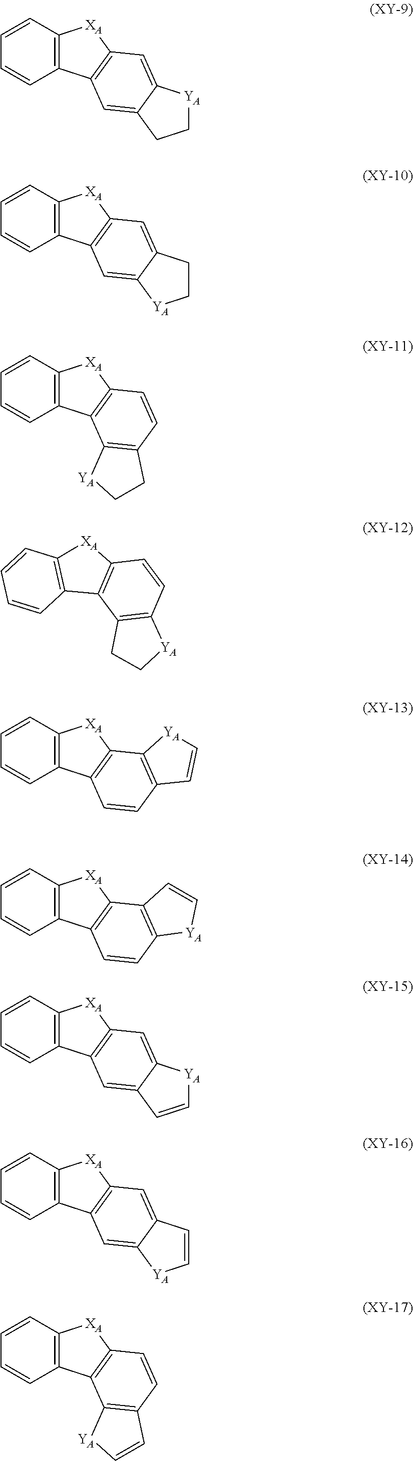

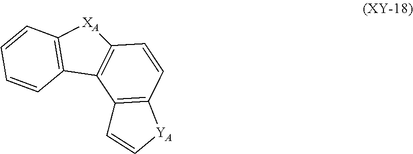

In the formulas (XY-1) to (XY-18), X_{A} and Y_{A} are independently an oxygen atom, a sulfur atom, NH or CH₂. However, at least one of X_{A} and Y_{A} is an oxygen atom, a sulfur atom or NH.

The heterocyclic ring represented by the formulas (XY-1) to (XY-18) becomes a monovalent heterocyclic group including a bond at an arbitrary position.

An expression “the monovalent group derived from the unsubstituted heterocyclic ring represented by the formulas (XY-1) to (XY-18) has a substituent” refers to a case where the hydrogen atom bonded with the carbon atom which constitutes a skeleton of the formulas is substituted by a substituent, or a state in which X_{A} Or Y_{A} is NH or CH₂, and the hydrogen atom in the NH or CH₂ is replaced with a substituent.

Specific examples (specific example group G3) of the “substituted or unsubstituted alkyl group” include an unsubstituted alkyl group and a substituted alkyl group described below. (Here, the unsubstituted alkyl group refers to a case where the “substituted or unsubstituted alkyl group” is the “unsubstituted alkyl group,” and the substituted alkyl group refers to a case where the “substituted or unsubstituted alkyl group” is the “substituted alkyl group”). Hereinafter, the case of merely “alkyl group” includes both the “unsubstituted alkyl group” and the “substituted alkyl group”.

The “substituted alkyl group” refers to a case where the “unsubstituted alkyl group” has a substituent, and specific examples thereof include a group in which the “unsubstituted alkyl group” has a substituent, and a substituted alkyl group described below. It should be noted that examples of the “unsubstituted alkyl group” and examples of the “substituted alkyl group” listed in this specification are merely one example, and the “substituted alkyl group” described in this specification also includes a group in which “unsubstituted alkyl group” has a substituent further has a substituent, a group in which “substituted alkyl group” further has a substituent, and the like.

An unsubstituted alkyl group:
a methyl group,
an ethyl group,
a n-propyl group,
an isopropyl group,
a n-butyl group,
an isobutyl group,
a s-butyl group, and
a t-butyl group.

A substituted alkyl group:
a heptafluoropropyl group (including an isomer),
a pentafluoroethyl group,
a 2,2,2-trifluoroethyl group, and
a trifluoromethyl group.

Specific examples (specific example group G4) of the “substituted or unsubstituted alkenyl group” include an unsubstituted alkenyl group and a substituted alkenyl group described below. (Here, the unsubstituted alkenyl group refers to a case where the “substituted or unsubstituted alkenyl group” is the “unsubstituted alkenyl group,” and the substituted alkenyl group refers to a case where the “substituted or unsubstituted alkenyl group” is the “substituted alkenyl group”). Hereinafter, the case of merely “alkenyl group” includes both the “unsubstituted alkenyl group” and the “substituted alkenyl group”.

The “substituted alkenyl group” refers to a case where the “unsubstituted alkenyl group” has a substituent, and specific examples thereof include a group in which the “unsubstituted alkenyl group” has a substituent, and a substituted alkenyl group described below. It should be noted that examples of the “unsubstituted alkenyl group” and examples of the “substituted alkenyl group” listed in this specification are merely one example, and the “substituted alkenyl group” described in this specification also includes a group in which “unsubstituted alkenyl group” has a substituent further has a substituent, a group in which “substituted alkenyl group” further has a substituent, and the like.

An unsubstituted alkenyl group and a substituted alkenyl group:
a vinyl group,
an allyl group,
a 1-butenyl group,
a 2-butenyl group,
a 3-butenyl group,
a 1,3-butanedienyl group,
a 1-methylvinyl group,
a 1-methylallyl group,
a 1,1-dimethylallyl group,
a 2-methylallyl group, and
a 1,2-dimethylallyl group.

Specific examples (specific example group G5) of the “substituted or unsubstituted alkynyl group” include an unsubstituted alkynyl group described below. (Here, the unsubstituted alkynyl group refers to a case where the “substituted or unsubstituted alkynyl group” is the “unsubstituted alkynyl group”). Hereinafter, a case of merely “alkynyl group” includes both the “unsubstituted alkynyl group” and the “substituted alkynyl group”.

The “substituted alkynyl group” refers to a case where the “unsubstituted alkynyl group” has a substituent, and specific examples thereof include a group in which the “unsubstituted alkynyl group” described below has a substituent.

An unsubstituted alkynyl group:
an ethynyl group.

Specific examples (specific example group G6) of the “substituted or unsubstituted cycloalkyl group” described in this specification include an unsubstituted cycloalkyl group and a substituted cycloalkyl group described below. (Here, the unsubstituted cycloalkyl group refers to a case where the “substituted or unsubstituted cycloalkyl group” is the “unsubstituted cycloalkyl group,” and the substituted cycloalkyl group refers to a case where the “substituted or unsubstituted cycloalkyl group” is the “substituted cycloalkyl group”). Hereinafter, a case of merely “cycloalkyl group” includes both the “unsubstituted cycloalkyl group” and the “substituted cycloalkyl group”.

The “substituted cycloalkyl group” refers to a case where the “unsubstituted cycloalkyl group” a the substituent, and specific examples thereof include a group in which the “unsubstituted cycloalkyl group” has a substituent, and a substituted cycloalkyl group described below. It should be noted that examples of the “unsubstituted cycloalkyl group” and examples of the “substituted cycloalkyl group” listed in this specification are merely one example, and the “substituted cycloalkyl group” described in this specification also includes a group in which “unsubstituted cycloalkyl group” has a substituent further has a substituent, a group in which “substituted cycloalkyl group” further has a substituent, and the like.

An unsubstituted aliphatic ring group:
a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

A substituted cycloalkyl group:
a 4-methylcyclohexyl group.

Specific examples (specific example group G7) of the group represented by
—Si(R₉₀₁)(R₉₀₂)(R₉₀₃) described in this specification include
—Si(G1)(G1)(G1),
—Si(G1)(G2)(G2),
—Si(G1)(G1)(G2),
—Si(G2)(G2)(G2),
—Si(G3)(G3)(G3),
—Si(G5)(G5)(G5) and
—Si(G6)(G6)(G6).

In which,
G1 is the “aryl group” described in the specific example group G1.
G2 is the “heterocyclic group” described in the specific example group G2.
G3 is the “alkyl group” described in the specific example group G3.
G5 is the “alkynyl group” described in the specific example group G5.
G6 is the “cycloalkyl group” described in the specific example group G6.

Specific examples (specific example group G8) of the group represented by
—O—(R₉₀₄) described in this specification include
—O(G1),
—O(G2),
—O(G3) and
—O(G6).

In which,
G1 is the “aryl group” described in the specific example group G1.
G2 is the “heterocyclic group” described in the specific example group G2.
G3 is the “alkyl group” described in the specific example group G3.
G6 is the “cycloalkyl group” described in the specific example group G6.

Specific examples (specific example group G9) of the group represented by
—S—(R₉₀₅) described in this specification include
—S(G1),
—S(G2),
—S(G3) and
—S(G6).

In which,
G1 is the “aryl group” described in the specific example group G1.
G2 is the “heterocycle group” described in the specific example group G2.
G3 is the “alkyl group” described in the specific example group G3.
G6 is the “cycloalkyl group” described in the specific example group G6.

Specific examples (specific example group G10) of the group represented by —N(R₉₀₆)(R₉₀₇) described in this specification include
—N(G1)(G1),
—N(G2)(G2),
—N(G1)(G2),
—N(G3)(G3) and
—N(G6) (G6).

In which,
G1 is the “aryl group” described in the specific example group G1.
G2 is the “heterocycle group” described in the specific example group G2.
G3 is the “alkyl group” described in the specific example group G3.
G6 is the “cycloalkyl group” described in the specific example group G6.

Specific examples (specific example group G11) of the “halogen atom” described in this specification include a fluorine atom, a chlorine atom, a bromine atom and an iodine atom.

Specific examples of the “alkoxy group” described in this specification include a group represented by —O(G3), where G3 is the “alkyl group” described in the specific example group G3. The number of carbon atoms of the “unsubstituted alkoxy group” are 1 to 50, preferably 1 to 30, and more preferably 1 to 18, unless otherwise specified.

Specific examples of the “alkylthio group” described in this specification include a group represented by —S(G3), where G3 is the “alkyl group” described in the specific example group G3. The number of carbon atoms of the “unsubstituted alkylthio group” are 1 to 50, preferably 1 to 30, and more preferably 1 to 18, unless otherwise specified.

Specific examples of the “aryloxy group” described in this specification include a group represented by —O(G1), where G1 is the “aryl group” described in the specific example group G1. The number of ring carbon atoms of the “unsubstituted aryloxy group” are 6 to 50, preferably 6 to 30, and more preferably 6 to 18, unless otherwise specified.

Specific examples of the “arylthio group” described in this specification include a group represented by —S(G1), where G1 is the “aryl group” described in the specific example group G1. The number of ring carbon atoms of the “unsubstituted arylthio group” are 6 to 50, preferably 6 to 30, and more preferably 6 to 18, unless otherwise specified.

Specific examples of the “aralkyl group” described in this specification include a group represented by -(G3)-(G1), where G3 is the “alkyl group” described in the specific example group G3, and G1 is the “aryl group” described in the specific example group G1. Accordingly, the “aralkyl group” is one embodiment of the “substituted alkyl group” substituted by the “aryl group”. The number of carbon atoms of the “unsubstituted aralkyl group,” which is the “unsubstituted alkyl group” substituted by the “unsubstituted aryl group,” are 7 to 50, preferably 7 to 30, and more preferably 7 to 18, unless otherwise specified.

Specific example of the “aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

The substituted or unsubstituted aryl group described in this specification is, unless otherwise specified, preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-diphenylfluorenyl group, or the like.

The substituted or unsubstituted heterocyclic group described in this specification is, unless otherwise specified, preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazole-9-yl group, a phenylcarbazol-9-yl group, a phenyltriazinyl group, a biphenylyltriazinyl group, diphenyltriazinyl group, a phenyldibenzofuranyl group, a phenyldibenzothiophenyl group, an indrocarbazolyl group, a pyrazinyl group, a pyridazinyl group, a quinazolinyl group, a cinnolinyl group, a phthalazinyl group, a quinoxalinyl group, a pyrrolyl group, an indolyl group, a pyrrolo[3,2,1-jk]carbazolyl group, a furanyl group, a benzofuranyl group, a thiophenyl group, a benzothiophenyl group, a pyrazolyl group, an imidazolyl group, a benzimidazolyl group, a triazolyl group, an oxazolyl group, a benzoxazolyl group, a thiazolyl group, a benzothiazolyl group, an isothiazolyl group, a benzisothiazolyl group, a thiadiazolyl group, an isoxazolyl group, a benzisoxazolyl group, a pyrrolidinyl group, a piperidinyl group, a piperazinyl group, an imidazolidinyl group, an indro[3,2,1-jk]carbazolyl group, a dibenzothiophenyl group, or the like.

The dibenzofuranyl group and the dibenzothiophenyl group as described above are specifically any group described below, unless otherwise specified.

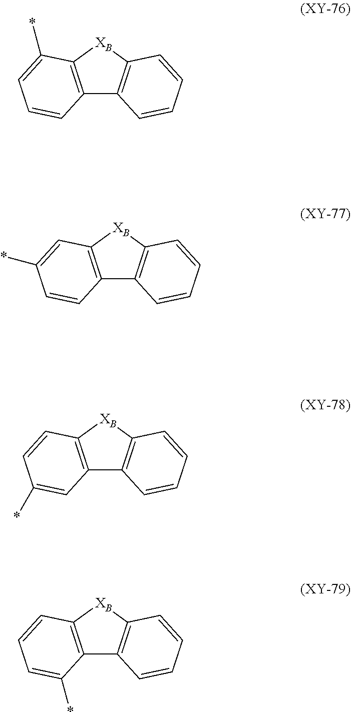

In the formulas (XY-76) to (XY-79), X_{B} is an oxygen atom or a sulfur atom.

The substituted or unsubstituted alkyl group described in this specification is, unless otherwise specified, preferably a methyl group, an ethyl group, a propyl group, an isopropyl group, a n-butyl group, an isobutyl group, a t-butyl group, or the like.

The “substituted or unsubstituted arylene group” descried in this specification refers to a group in which the above-described “aryl group” is converted into divalence, unless otherwise specified. Specific examples (specific example group G12) of the “substituted or unsubstituted arylene group” include a group in which the “aryl group” described in the specific example group G1 is converted into divalence. Namely, specific examples (specific example group G12) of the “substituted or unsubstituted arylene group” refer to a group derived from the “aryl group” described in specific example group G1 by removal of one hydrogen atom bonded to the ring carbon atoms thereof.

Specific examples (specific example group G13) of the “substituted or unsubstituted divalent heterocyclic group” include a group in which the “heterocyclic group” described in the specific example group G2 is converted into divalence. Namely, specific examples (specific example group G13) of the “substituted or unsubstituted divalent heterocyclic group” refer to a group derived from the “heterocyclic group” described in specific example group G2 by removal of one hydrogen atom bonded to the ring atoms thereof.

Specific examples (specific example group G14) of the “substituted or unsubstituted alkylene group” include a group in which the “alkyl group” described in the specific example group G3 is converted into divalence. Namely, specific examples (specific example group G14) of the “substituted or unsubstituted alkylene group” refer to a group derived from the “alkyl group” described in specific example group G3 by removal of one hydrogen atom bonded to the carbon atoms constituting the alkane structure thereof.

The substituted or unsubstituted arylene group described in this specification is any group described below, unless otherwise specified.

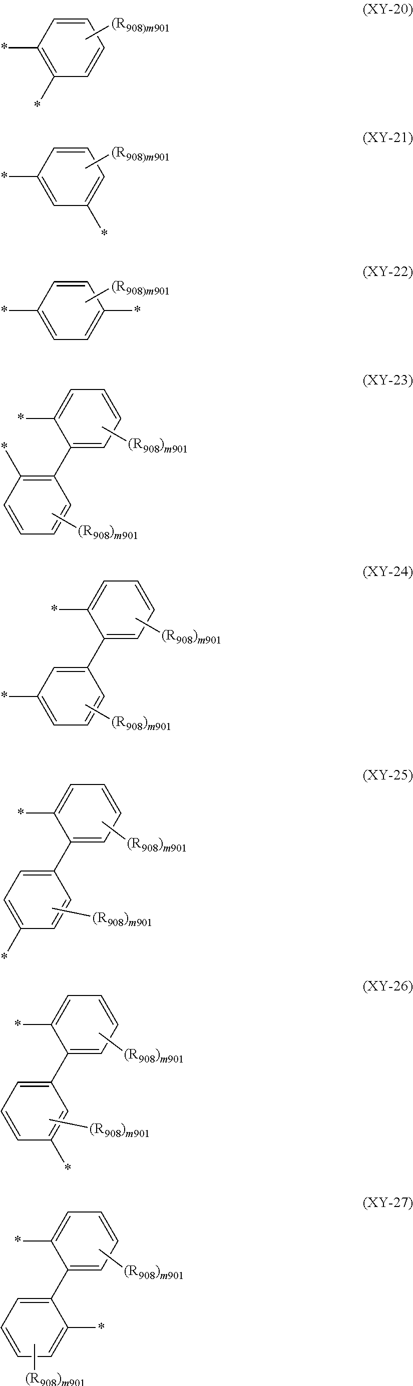

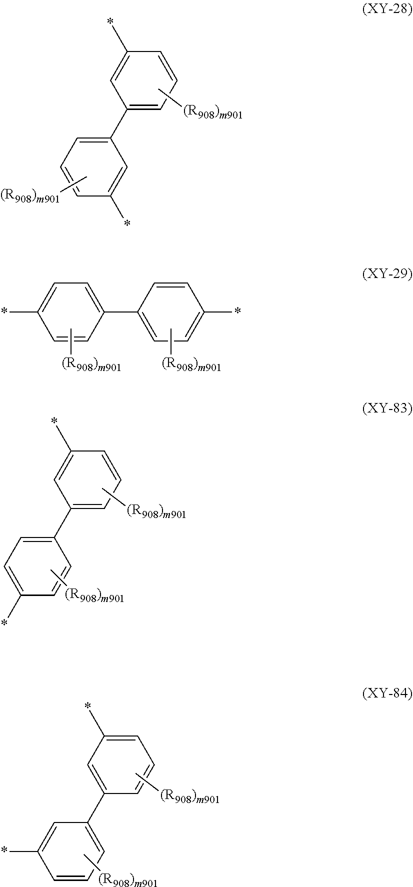

In the formulas (XY-20) to (XY-29), (XY-83) and (XY-84), R₉₀₈ is a substituent.

Then, m901 is an integer of 0 to 4, and when m901 is 2 or more, a plurality of R₉₀₈ may be the same with or different from each other.

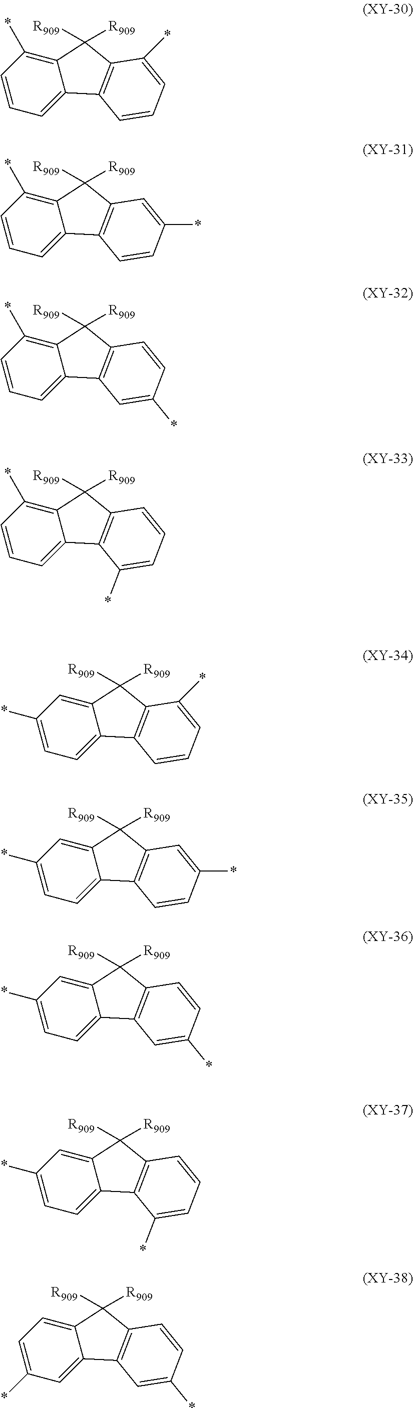

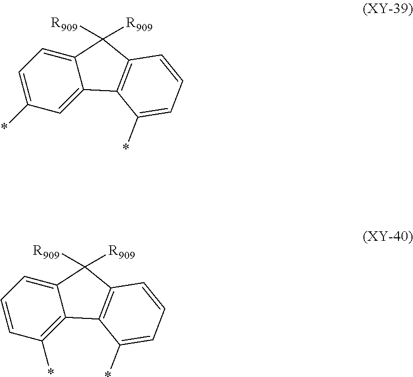

In the formulas (XY-30) to (XY-40), R₉₀₉ is independently a hydrogen atom or a substituent. Two of R₉₀₉ may form a ring by bonding with each other through a single bond.

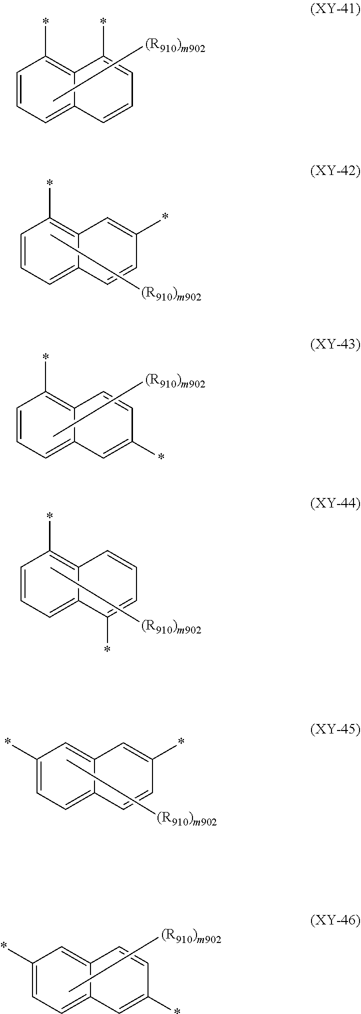

In the formulas (XY-41) to (XY-46), R₉₁₀ is a substituent.

Then, m902 is an integer of 0 to 6. When m902 is 2 or more, a plurality of R₉₁₀ may be the same with or different from each other.

The substituted or unsubstituted divalent heterocyclic group described in this specification is preferably any group described below, unless otherwise specified.

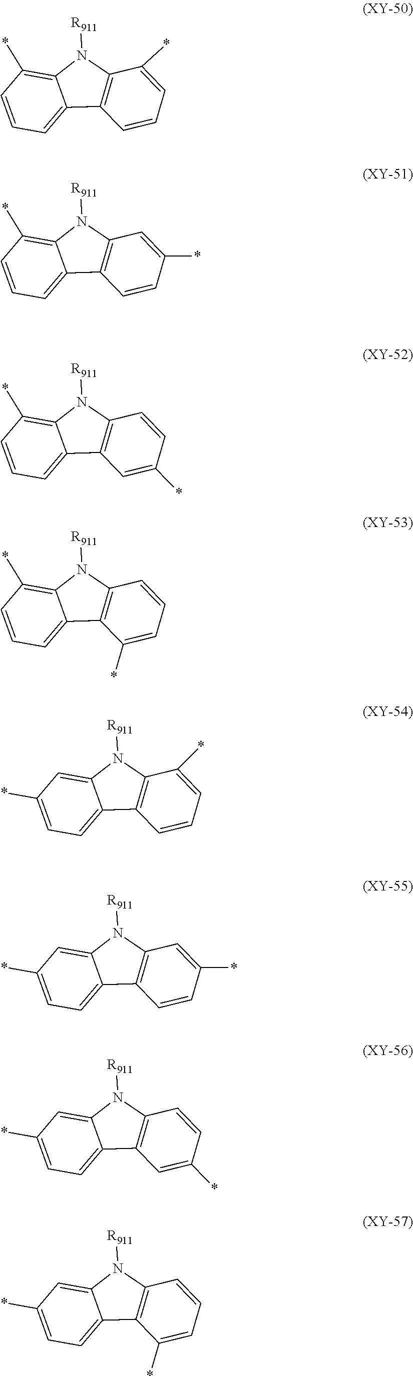

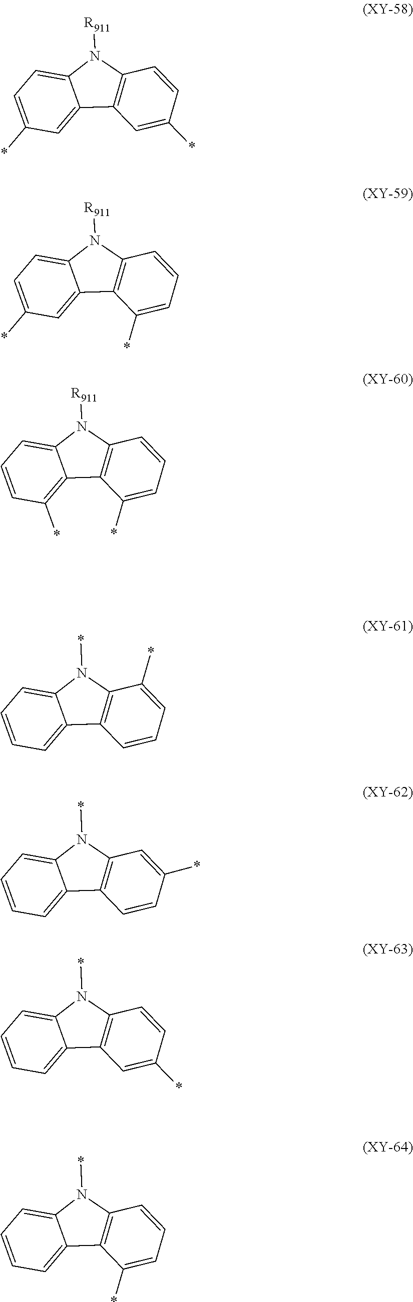

In the formulas (XY-50) to (XY-60), R₉₁₁ is a hydrogen atom or a substituent.

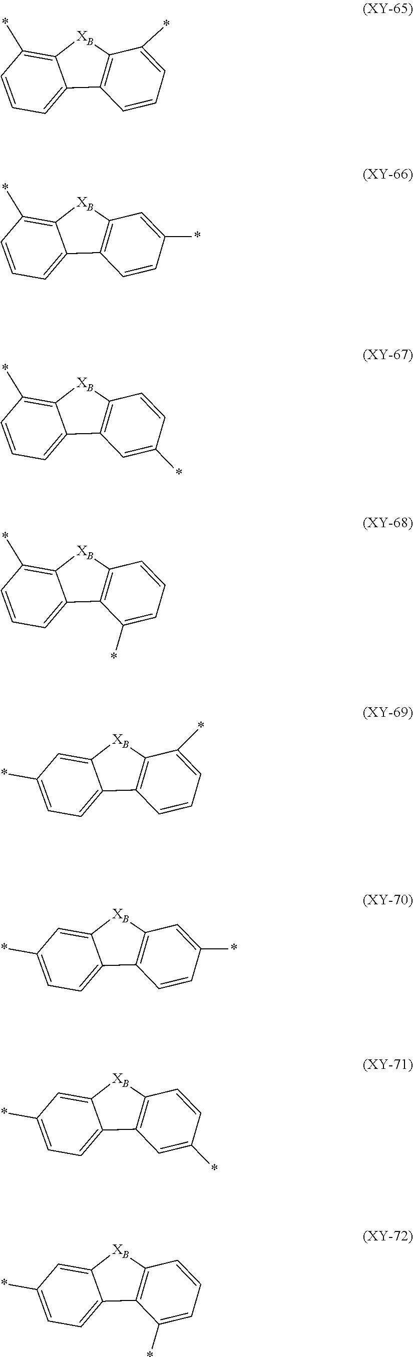

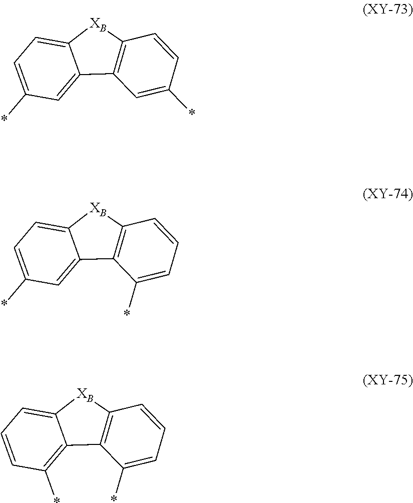

In the formulas (XY-65) to (XY-75), X_{B} is an oxygen atom or a sulfur atom.

In this specification, a case where “one or more sets of two or more groups adjacent to each other form a substituted or unsubstituted and saturated or unsaturated ring by bonding with each other” will be described by taking, as an example, a case of an anthracene compound represented by the following formula (XY-80) in which a mother skeleton is an anthracene ring.

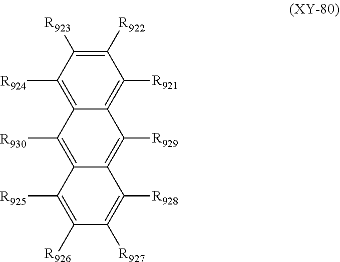

For example, two adjacent to each other into one set when “one or more sets of two or more groups adjacent to each other form the ring by bonding with each other” among R₉₂₁ to R₉₃₀ include R₉₂₁ and R₉₂₂, R₉₂₂ and R₉₂₃, R₉₂₃ and R₉₂₄, R₉₂₄ and R₉₃₀, R₉₃₀ and R₉₂₅, R₉₂₅ and R₉₂₆, R₉₂₆ and R₉₂₇, R₉₂₇ and R₉₂₈, R₉₂₈ and R₉₂₉, and R₉₂₉ and R₉₂₁.

The above-described “one or more sets” means that two or more sets of two groups adjacent to each other may simultaneously form the ring. For example, a case where R₉₂₁ and R₉₂₂ form a ring A by bonding with each other, and simultaneously R₉₂₅ and R₉₂₆ form a ring B by bonding with each other is represented by the following formula (XY-81).

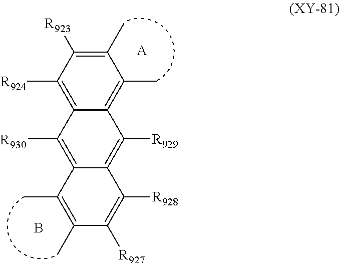

A case where “two or more groups adjacent to each other” form a ring means that, for example, R₉₂₁ and R₉₂₂ form a ring A by bonding with each other, and R₉₂₂ and R₉₂₃ form a ring C by bonding with each other. A case where the ring A and ring C sharing R₉₂₂ are formed, in which the ring A and the ring C are fused to the anthracene mother skeleton by three of R₉₂₁ to R₉₂₃ adjacent to each other, is represented by the following (XY-82).

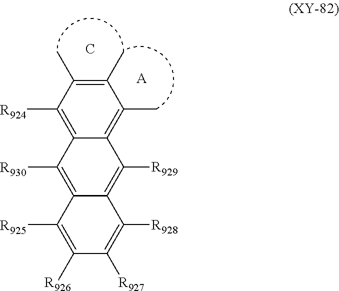

The rings A to C formed in the formulas (XY-81) and (XY-82) are a saturated or unsaturated ring.

A term “unsaturated ring” means an aromatic hydrocarbon ring or an aromatic heterocyclic ring. A term “saturated ring” means an aliphatic hydrocarbon ring or an aliphatic heterocyclic ring.

For example, the ring A formed by R₉₂₁ and R₉₂₂ being bonded with each other, represented by the formula (XY-81), means a ring formed by a carbon atom of the anthracene skeleton bonded with R₉₂₁, a carbon atom of the anthracene skeleton bonded with R₉₂₂, and one or more arbitrary elements. Specific examples include, when the ring A is formed by R₉₂₁ and R₉₂₂, a case where an unsaturated ring is formed of a carbon atom of an anthracene skeleton bonded with R₉₂₁, a carbon atom of the anthracene skeleton bonded with R₉₂₂, and four carbon atoms, in which a ring formed by R₉₂₁ and R₉₂₂ is formed into a benzene ring. Further, when a saturated ring is formed, the ring is formed into a cyclohexane ring.

Here, “arbitrary elements” are preferably a C element, a N element, an O element and a S element. In the arbitrary elements (for example, a case of the C element or the N element), the bond(s) that is(are) not involved in the formation of the ring may be terminated by a hydrogen atom, or may be substituted by an arbitrary substituent. When the ring contains the arbitrary elements other than the C element, the ring to be formed is a heterocyclic ring.

The number of “one or more arbitrary elements” forming the saturated or unsaturated ring is preferably 2 or more and 15 or less, more preferably 3 or more and 12 or less, and further preferably 3 or more and 5 or less.

As specific examples of the aromatic hydrocarbon ring, a structure in which the aryl group described in specific example group G1 is terminated with a hydrogen atom may be mentioned.

As specific examples of the aromatic heterocyclic ring, a structure in which the aromatic heterocyclic group described in specific example group G2 is terminated with a hydrogen atom may be mentioned.

As specific examples of the aliphatic hydrocarbon ring, a structure in which the cycloalkyl group described in specific example group G6 is terminated with a hydrogen atom may be mentioned.

When the above-described “saturated or unsaturated ring” has a substituent, the substituent is an “arbitrary substituent” as described below, for example. When the above-mentioned “saturated or unsaturated ring” has a substituent, specific examples of the substituent refer to the substituents described in above-mentioned “the substituent described herein”.

In one embodiment of this specification, the substituent (hereinafter, referred to as an “arbitrary substituent” in several cases) in the case of the “substituted or unsubstituted” is a group selected from the group consisting of
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅)
—N(R₉₀₆)(R₉₀₇)
wherein,
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and when two or more of R₉₀₁ to R₉₇ exist, two or more of R₉₀₁ to R₉₀₇ may be the same with or different from each other,
a halogen atom, a cyano group, a nitro group,
an unsubstituted aryl group including 6 to 50 ring carbon atoms, and
an unsubstituted monovalent heterocyclic group including 5 to 50 ring atoms.

In one embodiment, the substituent in the case of “substituted or unsubstituted” is a group selected from the group consisting of
an alkyl group including 1 to 50 carbon atoms,
an aryl group including 6 to 50 ring carbon atoms, and
a monovalent heterocyclic group including 5 to 50 ring atoms.

In one embodiment, the substituent in the case of “substituted or unsubstituted” is a group selected from the group consisting of
an alkyl group including 1 to 18 carbon atoms,
an aryl group including 6 to 18 ring carbon atoms, and
a monovalent heterocyclic group including 5 to 18 ring atoms.

Specific examples of each group of the arbitrary substituent described above are as described above.

In this specification, unless otherwise specified, the saturated or unsaturated ring (preferably substituted or unsubstituted and saturated or unsaturated five-membered or six-membered ring, more preferably a benzene ring) may be formed by the arbitrary substituents adjacent to each other.

In this specification, unless otherwise specified, the arbitrary substituent may further have the substituent. Specific examples of the substituent that the arbitrary substituent further has include to the ones same as the arbitrary substituent described above.

### [Compound Represented by the Formula (A1)]

A novel compound of an aspect of the invention is represented by the following formula (A1).

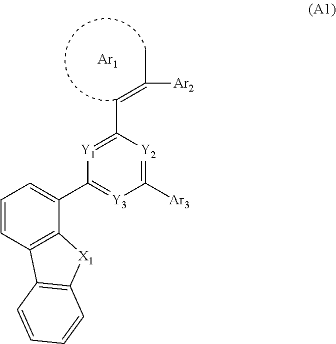

In the formula (A1),
X₁ is O or S;
Y₁, Y₂, and Y₃ are independently CH or N;
provided that two or more of Y₁, Y₂, and Y₃ are N;
Ar₁ is an aryl group including 6 to 50 ring carbon atoms, comprising a benzene ring having at least substituent Ar₂ at the ortho-position;
Ar₂ is a substituted or unsubstituted aryl group including 6 to 50 ring carbon atoms;
Ar₁ and Ar₂ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring;
Ar₃ is a group selected from the group consisting of a group represented by the following formula (A2-1), a group represented by the following formula (A2-2), a group represented by the following formula (A2-3), and a group represented by the following formula (A2-4).

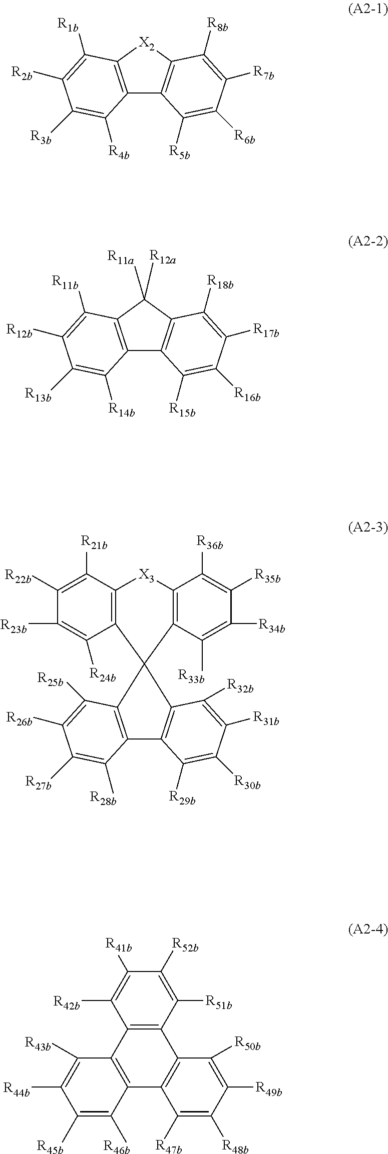

In the formula (A2-1),
X₂ is O or S;
one of R_{1b} to R_{8b} is a single bond bonding with the carbon atom between Y₂ and Y₃, and the others are hydrogen atoms;
wherein in the formula (A2-2),
R₁₁ₐ and R₁₂ₐ form a substituted or unsubstituted, saturated or unsaturated ring by bonding with each other, or do not form a substituted or unsubstituted, saturated or unsaturated ring;
one of R_{11b} to R_{18b} is a single bond bonding with the carbon atom between Y₂ and Y₃;
R₁₁ₐ and R₁₂ₐ which do not form the substituted or unsubstituted, saturated or unsaturated ring, and R_{11b} to R_{18b} which are not the single bond are independently a hydrogen atom,
   a substituted or unsubstituted alkyl group including 1 to 50 carbon atoms, or
   a substituted or unsubstituted aryl group including 6 to 50 ring carbon atoms;
wherein in the formula (A2-3),
one of R_{21b} to R_{36b} is a single bond bonding with the carbon atom between Y₂ and Y₃;
X₃ is NR₂₁ₐ, CR₂₂ₐR₂₃ₐ, O, or S;
R₂₁ₐ form a substituted or unsubstituted, saturated or unsaturated ring by bonding either or both of R_{21b} which is not a single bond or R_{36b} which is not a single bond, or does not form the substituted or unsubstituted, saturated or unsaturated ring;
R_{22b} to R_{35b} which are not single bonds, R_{21b} and R_{36b} which are not single bonds and which do not form the substituted or unsubstituted, saturated or unsaturated ring, R₂₁ₐ which does not form the substituted or unsubstituted, saturated or unsaturated ring, and R₂₂ₐ and R₂₃ₐ are independently a hydrogen atom,
   a substituted or unsubstituted alkyl group including 1 to 50 carbon atoms, or
   a substituted or unsubstituted aryl group including 6 to 50 ring carbon atoms;
wherein in the formula (A2-4),
one of R_{41b} to R_{52b} is a single bond bonding with the carbon atom between Y₂ and Y₃; and
R_{41b} to R_{52b} which are not the single bond are independently a hydrogen atom,
   a substituted or unsubstituted alkyl group including 1 to 50 carbon atoms, or
   a substituted or unsubstituted aryl group including 6 to 50 ring carbon atoms.

The compound represented by the formula (A1) has a low affinity value, when the compound represented by the formula (A1) is used as a material for the electron-transporting layer, the electron-injecting property into the emitting layer is improved, resulting in an organic EL device with high luminous efficiency and/or low drive voltage.

As mentioned above, a “hydrogen atom” as used in this specification includes a protium atoms, a deuterium atom, and a tritium atom. Thus, the compound represented by the formula (A1) may have a naturally derived deuterium atom.

A deuterium atom may also be intentionally introduced into the compound represented by the formula (A1) by using, as a raw material compound, a compound in which some or all of the hydrogen atoms of the compound are deuterium atoms (hereinafter referred to as “deuterated compound”). Thus, in one embodiment, the compound represented by the formula (A1) has at least one deuterium atom (hereinafter, the embodiment of the compound represented by the formula (A1) having a deuterium atom is referred to as “Embodiment D”). That is, the compound represented by the formula (A1) is a compound represented by the formula (A1) or a preferred embodiment thereof, and at least one of the hydrogen atoms possessed by the compound may be a deuterium atom. The deuterium atom may be a hydrogen atom at any position of the compound represented by the formula (A1) or a preferred embodiment thereof.

The deuterated ratio (the ratio of the number of deuterium atoms to the total number of hydrogen atoms in the compound represented by the formula (A1)) of the compound represented by the formula (A1) of Embodiment D depends on the deuterated ratio of the raw material compound used.

Since it is generally difficult to achieve a deuterated ratio of 100% for all raw material compounds used, the deuterated ratio of the compound represented by the formula (A1) is preferably less than 100%.

The deuterated ratio (the ratio of the number of deuterium atoms to the total number of hydrogen atoms in the compound represented by the formula (A1)) of the compound represented by the formula (A1) in Embodiment D is 1% or more, preferably 3% or more, more preferably 5% or more, and still more preferably 10% or more.

The compound represented by the formula (A1) of Embodiment D may be a mixture containing a deuterated compound and a non-deuterated compound having the same chemical structure, or a mixture of two or more compounds having different deuterated ratios. The deuterated ratio (the ratio of the number of deuterium atoms to the total number of hydrogen atoms in the compound represented by the formula (A1) contained in the mixture) of such a mixture is 1% or more, preferably 3% or more, more preferably 5% or more, still more preferably 10% or more, and less than 100%.

In the compound represented by the formula (A1) of Embodiment D, H (hydrogen atom) in the case where one of Y₁, Y₂, and Y₃ is CH may be a deuterium atom.

In the compound represented by the formula (A1) in Embodiment D, at least one of the hydrogen atoms possessed by the aryl group represented by Ar₁ and Ar₂ may be a deuterium atom. The deuterated ratio (the ratio of the number of deuterium atoms to the total number of hydrogen atoms possessed by the aryl group represented by Ar₁ and Ar₂) is 1% or more, preferably 3% or more, more preferably 5% or more, still more preferably 10% or more, and less than 100%.

In the compound represented by the formula (A1) or (1) in Embodiment D, at least one hydrogen atom selected from the hydrogen atoms possessed by the group consisting of the group represented by the formula (A2-1), the group represented by the following formula (A2-2), the group represented by the following formula (A2-3), and the group represented by the following formula (A2-4), which are represented by Ar₃, may be a deuterium atom. The deuterated ratio (the ratio of the number of deuterium atoms to the total number of hydrogen atoms possessed by the group represented by Ar₃) is 1% or more, preferably 3% or more, more preferably 5% or more, still more preferably 10% or more, and less than 100%.

In one embodiment, the group represented by the formula (A2-1) is a group represented by the following formula (A2-1-1) or (A2-1-2).

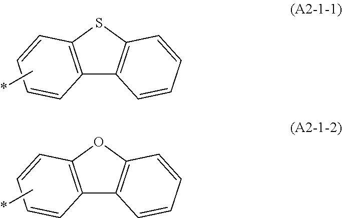

In the formulas (A2-1-1) and (A2-1-2), *s represent a single bond bonding with the carbon atom between Y₂ and Y₃.

In one embodiment, one of R_{12b}, R_{13b}, R_{16b}, and R_{17b} in the formula (A2-2) is a single bond bonding with the carbon atom between Y₂ and Y₃.

In one embodiment, one of R_{14b} and R_{15b} in the formula (A2-2) is a single bond bonding with the carbon atom between Y₂ and Y₃.

In one embodiment, one of R_{12b} and R_{17b} in the formula (A2-2) is a single bond bonding with the carbon atom between Y₂ and Y₃.

In one embodiment, the group represented by the formula (A2-2) is selected from the group represented by the following formula (A2-2-1), the group represented by the following formula (A2-2-2), and the group represented by the following formula (A2-2-3).

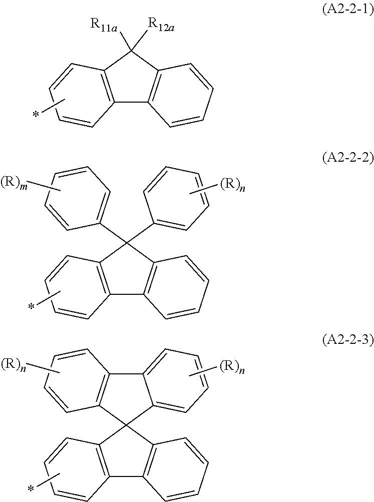

In formulas (A2-2-1) to (A2-2-3), R₁₁ₐ and R₁₂ₐ are as defined in the formula (A1).
*s represent a single bond bonding with the carbon atom between Y₂ and Y₃.
   R's are a substituent.
   m's are an integer of 0 to 5.
   n's are an integer of 0 to 4.

In one embodiment, the group represented by the formula (A2-3) is selected from the group represented by the following formula (A2-3-1), the group represented by the following formula (A2-3-2), the group represented by the following formula (A2-3-3), and the group represented by the following formula (A2-3-4).

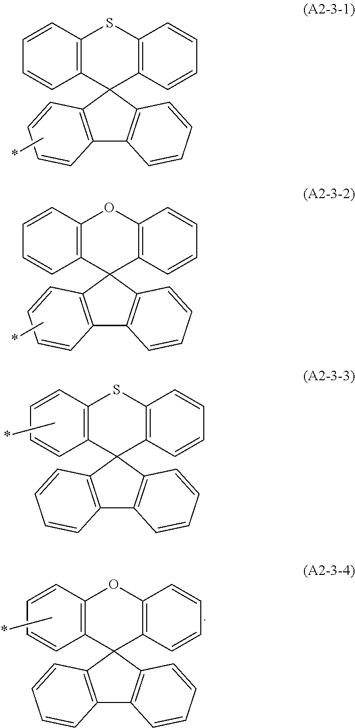

In the formulas (A2-3-1) to (A2-3-4), *s represent a single bond bonding with the carbon atom between Y₂ and Y₃.

In one embodiment, the group represented by the formula (A2-3) is selected from the group represented by the following formula (A2-3-5), and the group represented by the following formula (A2-3-6).

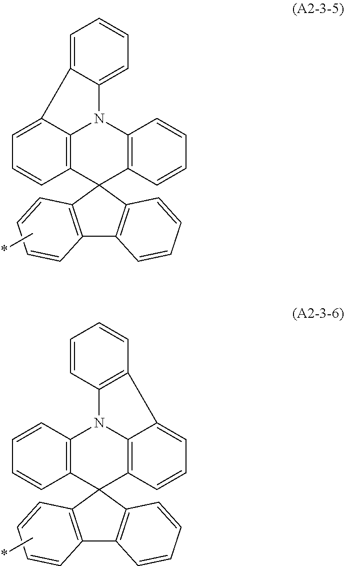

In the formulas (A2-3-5) and (A2-3-6), *s represent a single bond bonding with the carbon atom between Y₂ and Y₃.

In one embodiment, the group represented by the formula (A2-4) is a group represented by the following formula (A2-4-1).

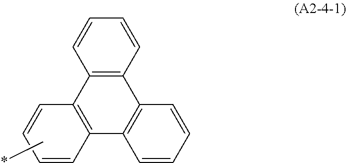

In the formula (A2-4-1), * represents a single bond bonding with the carbon atom between Y₂ and Y₃.

In one embodiment, the group represented by the formula (A1) is a group represented by the following formula (A3).

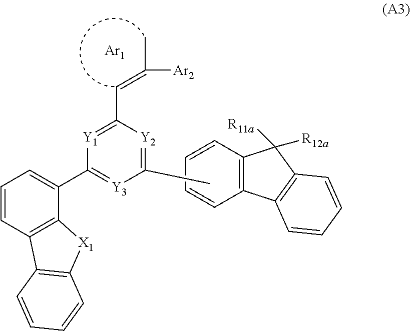

In the formula (A3), X₁, Y₁ to Y₃, Ar₁, and Ar₂ are as defined in formula (A1);
R₁₁ₐ and R₁₂ₐ form a substituted or unsubstituted, saturated or unsaturated ring by bonding with each other, or do not form a substituted or unsubstituted, saturated or unsaturated ring;
R₁₁ₐ and R₁₂ₐ which do not form the substituted or unsubstituted, saturated or unsaturated ring are independently
   a hydrogen atom,
   a substituted or unsubstituted alkyl group including 1 to 50 carbon atoms, or
   a substituted or unsubstituted aryl group including 6 to 50 ring carbon atoms.

In one embodiment, the compound represented by the formula (A3) is a compound represented by the following formula (A4-1) or (A4-2).

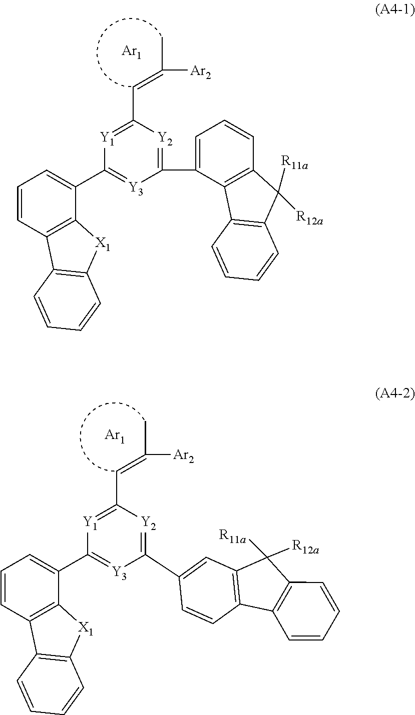

In the formulas (A4-1) and (A4-2), X₁, Y₁ to Y₃, Ar₁, Ar₂, R₁₁ₐ, and R₁₂ₐ are as defined in formula (A3).

In one embodiment, the compound represented by the formula (A3) is a compound represented by the following formula (A5-1) or (A5-2).

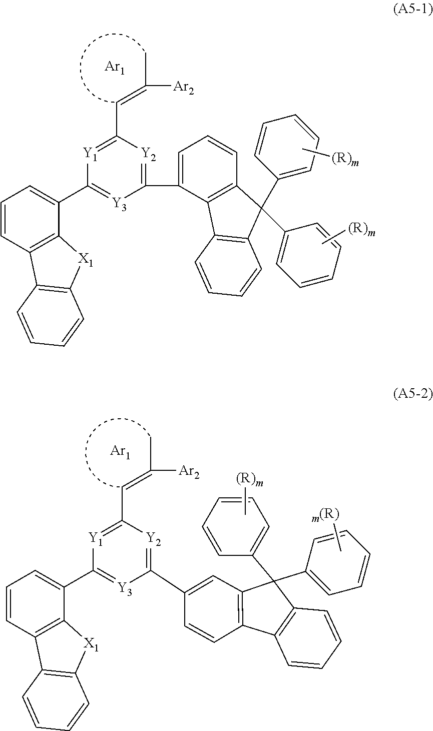

In the formulas (A5-1) and (A5-2), X₁, Y₁ to Y₃, Ar₁, and Ar₂ are as defined in formula (A3);
R's are a substituent;
m's are an integer of 0 to 5.

In one embodiment, the compound represented by the formula (A3) is a compound represented by the following formula (A6).

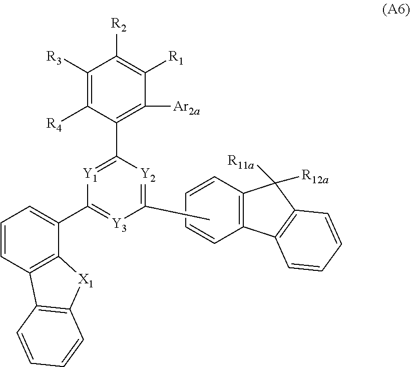

In the formula (A6), X₁, Y₁ to Y₃, R₁₁ₐ, and R₁₂ₐ are as defined in formula (A3);
Ar₂ₐ is a substituted or unsubstituted aryl group including 6 to 50 ring carbon atoms;
Ar₂ₐ, which is the aryl group, and R₁ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring;
one or more sets of adjacent two or more of R₁, which does not form the substituted or unsubstituted 5-membered hydrocarbon ring by bonding with Ar₂ₐ, and R₂ to R₄ form a substituted or unsubstituted, saturated or unsaturated ring or do not form a substituted or unsubstituted, saturated or unsaturated ring;
R₁ which does not form the substituted or unsubstituted 5-membered hydrocarbon ring by bonding with Ar₂ₐ and does not form the substituted or unsubstituted, saturated or unsaturated ring, and R₂ to R₄ which do not form the substituted or unsubstituted, saturated or unsaturated ring are independently a hydrogen atom,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃),

### —O—(R₉₀₄),

### —S—(R₉₀₅),

—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇: independently,
   a hydrogen atom,
   a substituted or unsubstituted alkyl group including 1 to 50 carbon atoms,
   a substituted or unsubstituted cycloalkyl group including 3 to 50 ring carbon atoms,
   a substituted or unsubstituted aryl group including 6 to 50 ring carbon atoms, or
   a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

In one embodiment, Ar₂ₐ in the formula (A6) is
a substituted or unsubstituted phenyl group,
a substituted or unsubstituted naphthyl group,
a substituted or unsubstituted anthryl group, or
a substituted or unsubstituted biphenyl group.

In one embodiment, all of R₁ to R₄ in the formula (A6) are hydrogen atoms.

In one embodiment, Ar₃ is the group represented by the formula (A2-1).

In one embodiment, the compound represented by the formula (A1) is a compound represented by the following formula (A7).

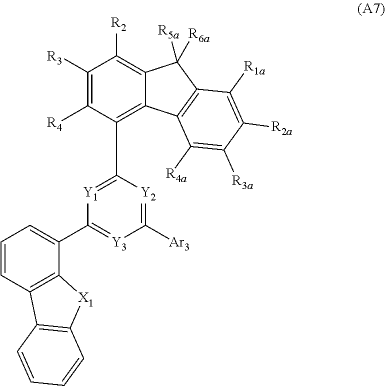

In the formula (A7), X₁ and Y₁ to Y₃ are as defined in formula (A1);
R₅ₐ and R₆ₐ form a substituted or unsubstituted, saturated or unsaturated ring by bonding with each other, or do not form a substituted or unsubstituted, saturated or unsaturated ring;
R₅ₐ and R₆ₐ which do not form the substituted or unsubstituted, saturated or unsaturated ring are independently
   a hydrogen atom,
   a substituted or unsubstituted alkyl group including 1 to 50 carbon atoms, or
   a substituted or unsubstituted aryl group including 6 to 50 ring carbon atoms; and
one or more sets of adjacent two or more of R₂ to R₄ and one or more sets of adjacent two or more of R₁ₐ to R₄ₐ form a substituted or unsubstituted, saturated or unsaturated ring by bonding with each other, or do not form a substituted or unsubstituted, saturated or unsaturated ring;
R₂ to R₄ and R₁ₐ to R₄ₐ which do not form the substituted or unsubstituted, saturated or unsaturated ring are independently
   a hydrogen atom,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃),

### —O—(R₉₀₄),

### —S—(R₉₀₅),

—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
   a hydrogen atom,
   a substituted or unsubstituted alkyl group including 1 to 50 carbon atoms,
   a substituted or unsubstituted cycloalkyl group including 3 to 50 ring carbon atoms,
   a substituted or unsubstituted aryl group including 6 to 50 ring carbon atoms, or
   a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

In one embodiment, Y₁ to Y₃ are N.

In one embodiment, X₁ is S.

### [Compound Represented by the Formula (1)]

A novel compound of an aspect of the invention is represented by the following formula (1). The compound represented by the following formula (1) is one embodiment of the compound represented by the formula (A1).

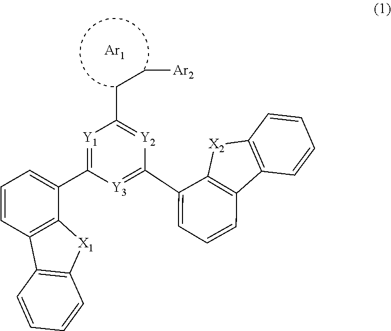

In the formula (1),
X₁ and X₂ are independently O or S;
Y₁, Y₂, and Y₃ are independently CH or N;
provided that two or more of Y₁, Y₂, and Y₃ are N;
Ar₁ is an aryl group including 6 to 50 ring carbon atoms having at least one substituent, comprising a benzene ring substituted by Ar₂ at least in the ortho-position;
Ar₂ is a substituted or unsubstituted aryl group including 6 to 50 ring carbon atoms;
Ar₁ and Ar₂ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring.

The compound represented by the formula (1) has a bulky structure, and due to the bulkiness of the structure, the compound represented by the formula (1) is also expected to have high electron mobility, good solubility, etc.

Specific examples of the “aryl group” of “an aryl group including 6 to 50 ring carbon atoms having at least one substituent, comprising a benzene ring substituted by Ar₂ at least in the ortho-position,” which is Ar₁ in the formula (1), include, for example, a phenyl group, a naphthyl group, an anthryl group, a phenanthryl group, a fluorenyl group, a naphthacenyl group, a pyrenyl group, a chrysenyl group, a triphenylethenyl group, a fluoranthenyl group, and the like.

Specific examples of a polycyclic fused aryl group formed by fusing of the “aryl group” of “an aryl group including 6 to 50 ring carbon atoms having at least one substituent, comprising a benzene ring substituted by Ar₂ at least in the ortho-position,” which is Ar₁, and the aryl group, which is Ar₂, via a 5-membered hydrocarbon ring include, for example, the group represented by the following formulas.

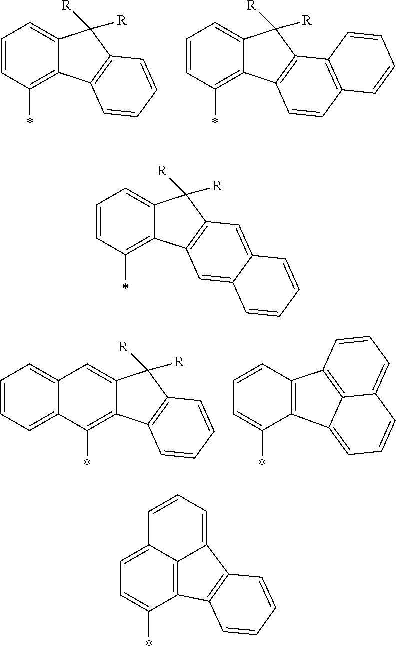

In the formulas, R's are a hydrogen atom or a substituent, and *s are the bonding position to the 6-membered ring containing Y₁ to Y₃.

The compound represented by the formula (1) is, more specifically, a compound represented by the following formula (2).

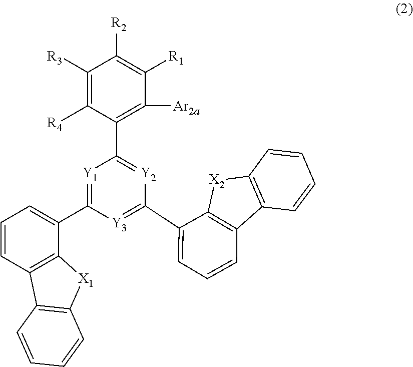

In the formula (2), X₁, X₂, and Y₁ to Y₃ are as defined in the formula (1);
Ar₂ₐ is a substituted or unsubstituted aryl group including 6 to 50 ring carbon atoms;
Ar₂ₐ, which is the aryl group, and R₁ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring;
one or more sets of adjacent two or more of R₁, which does not form the substituted or unsubstituted 5-membered hydrocarbon ring by bonding with Ar₂ₐ, and R₂ to R₄ form a substituted or unsubstituted, saturated or unsaturated ring or do not form a substituted or unsubstituted, saturated or unsaturated ring;
R₁ which does not form the substituted or unsubstituted 5-membered hydrocarbon ring by bonding with Ar₂ₐ and does not form the substituted or unsubstituted, saturated or unsaturated ring, and R₂ to R₄ which do not form the substituted or unsubstituted, saturated or unsaturated ring are independently
   a hydrogen atom,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃),

### —O—(R₉₀₄),

### —S—(R₉₀₅),

—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
   a hydrogen atom,
   a substituted or unsubstituted alkyl group including 1 to 50 carbon atoms,
   a substituted or unsubstituted cycloalkyl group including 3 to 50 ring carbon atoms,
   a substituted or unsubstituted aryl group including 6 to 50 ring carbon atoms, or
   a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

In the case where “an aryl group including 6 to 50 ring carbon atoms having at least one substituent, comprising a benzene ring substituted by Ar₂ at least in the ortho-position,” which is Ar₁ is a phenyl group, two ortho-positions are present, and one of the ortho-position is substituted by Ar₂ₐ, and the other is substituted by R₄ which does not form a ring. In the case where R₄ is a substituted or unsubstituted aryl group including 6 to 50 ring carbon atoms, Ar₁, which is a phenyl group, have the aryl group in two ortho-positions.

In one embodiment, the compound represented by the formula (1) is a compound represented by the following formula (2H).

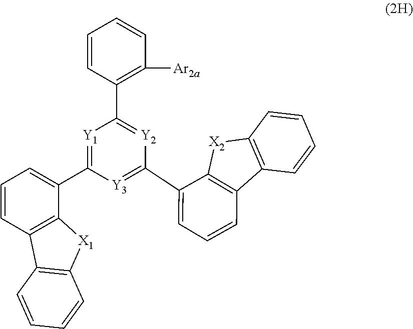

In the formula (2H), X₁, X₂, and Y₁ to Y₃ are as defined in formula (1);
Ar₂ₐ is a substituted or unsubstituted aryl group including 6 to 50 ring carbon atoms.

In one embodiment, the compound represented by the formula (1) is a compound represented by the following formula (3-1) or a compound represented by the following formula (3-2).

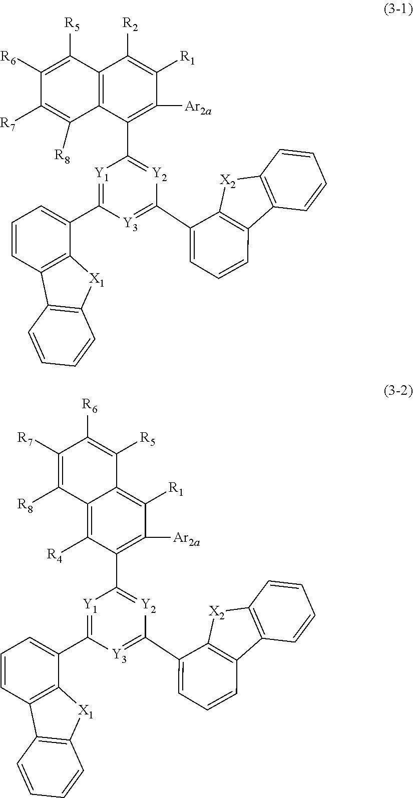

In the formulas (3-1) and (3-2), X₁, X₂, and Y₁ to Y₃ are as defined in the formula (1);
Ar₂ₐ is a substituted or unsubstituted aryl group including 6 to 50 ring carbon atoms;
Ar₂ₐ, which is the aryl group, and R₁ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring;
one or more sets of adjacent two or more of R₁, which does not form the substituted or unsubstituted 5-membered hydrocarbon ring by bonding with Ar₂ₐ, and R₂ and R₄ to R₈ form a substituted or unsubstituted, saturated or unsaturated ring or do not form a substituted or unsubstituted, saturated or unsaturated ring;
R₁ which does not form the substituted or unsubstituted 5-membered hydrocarbon ring by bonding with Ar₂ₐ and does not form the substituted or unsubstituted, saturated or unsaturated ring, and R₂ and R₄ to R₈ which do not form the substituted or unsubstituted, saturated or unsaturated ring are independently a hydrogen atom,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃),

### —O—(R₉₀₄),

### —S—(R₉₀₅),

—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
   a hydrogen atom,
   a substituted or unsubstituted alkyl group including 1 to 50 carbon atoms,
   a substituted or unsubstituted cycloalkyl group including 3 to 50 ring carbon atoms,
   a substituted or unsubstituted aryl group including 6 to 50 ring carbon atoms, or
   a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

In one embodiment, the compound represented by the formula (1) is a compound represented by the following formula (3H-1) or a compound represented by the following formula (3H-2).

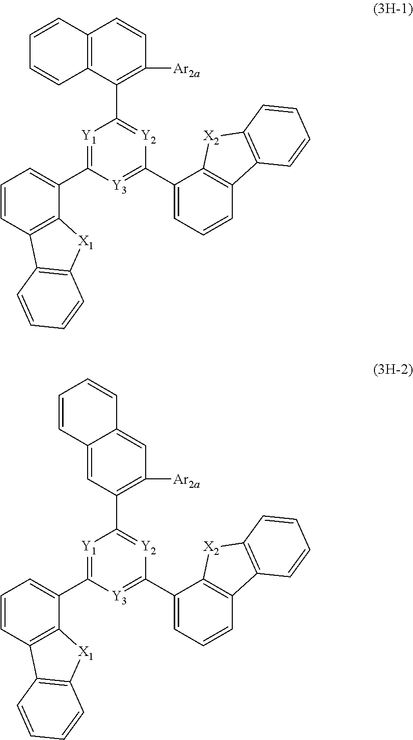

In the formulas (3H-1) and (3H-2), X₁, X₂, and Y₁ to Y₃ are as defined in formula (1);
Ar₂ₐ is a substituted or unsubstituted aryl group including 6 to 50 ring carbon atoms.

In one embodiment, the compound represented by the formula (1) is a compound represented by the following formula (4).

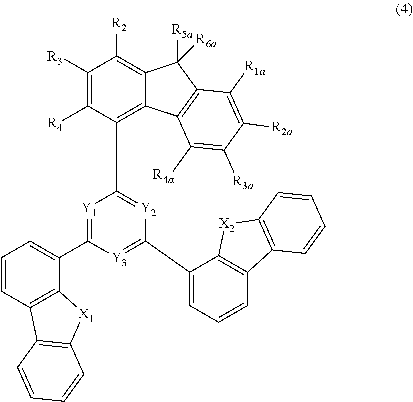

In the formula (4), X₁, X₂, and Y₁ to Y₃ are as defined in formula (1);
R₅ₐ and R₆ₐ form a substituted or unsubstituted, saturated or unsaturated ring by bonding with each other, or do not form a substituted or unsubstituted, saturated or unsaturated ring;
R₅ₐ and R₆ₐ which do not form the substituted or unsubstituted, saturated or unsaturated ring are independently a hydrogen atom,
   a substituted or unsubstituted alkyl group including 1 to 50 carbon atoms, or
   a substituted or unsubstituted aryl group including 6 to 50 ring carbon atoms; and
one or more sets of adjacent two or more of R₂ to R₄ and one or more sets of adjacent two or more of R₁ₐ to R₄ₐ form a substituted or unsubstituted, saturated or unsaturated ring by bonding with each other, or do not form a substituted or unsubstituted, saturated or unsaturated ring;
R₂ to R₄ and R₁ₐ to R₄ₐ which do not form the substituted or unsubstituted, saturated or unsaturated ring are independently
   a hydrogen atom,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃),

### —O—(R₉₀₄),

### —S—(R₉₀₅),

—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
   a hydrogen atom,
   a substituted or unsubstituted alkyl group including 1 to 50 carbon atoms,
   a substituted or unsubstituted cycloalkyl group including 3 to 50 ring carbon atoms,
   a substituted or unsubstituted aryl group including 6 to 50 ring carbon atoms, or
   a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

In one embodiment, the compound represented by the formula (1) is a compound represented by the following formula (4H).

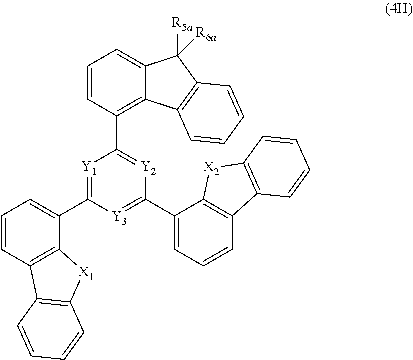

In the formula (4H), X₁, X₂, and Y₁ to Y₃ are as defined in formula (1);
R₅ₐ and R₆ₐ form a substituted or unsubstituted, saturated or unsaturated ring by bonding with each other, or do not form a substituted or unsubstituted, saturated or unsaturated ring; and
R₅ₐ and R₆ₐ which do not form the substituted or unsubstituted, saturated or unsaturated ring are independently a hydrogen atom,
   a substituted or unsubstituted alkyl group including 1 to 50 carbon atoms, or
   a substituted or unsubstituted aryl group including 6 to 50 ring carbon atoms.

In one embodiment, Ar₂ₐ is
a substituted or unsubstituted phenyl group,
a substituted or unsubstituted naphthyl group,
a substituted or unsubstituted anthryl group, or
a substituted or unsubstituted biphenyl group.

In one embodiment, the substituent of the “substituted or unsubstituted” is
a substituted or unsubstituted alkyl group including 1 to 50 carbon atoms, or
a substituted or unsubstituted aryl group including 6 to 50 ring carbon atoms.

In one embodiment, one of X₁ and X₂ is S and the other is O.

In one embodiment, both X₁ and X₂ are S.

When two of Y₁ to Y₃ are N, Y₁ and Y₂ may be N, Y₁ and Y₃ may be N, or Y₂ and Y₃ may be N.

In one embodiment, all of Y₁ to Y₃ are N.

In one embodiment, the compound represented by the formula (2) is a compound represented by the following formula (5).

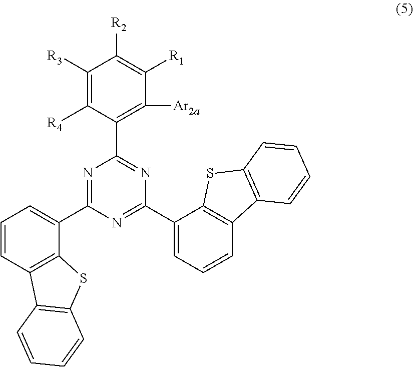

In the formula (5), R₁ to R₄ and Ar₂ₐ are as defined in the formula (2).

In one embodiment, X₁ and X₂ are S, Y₁ to Y₃ are N.

In one embodiment, X₁ and X₂ are S, Y₁ and Y₂ are N, and Y₃ is CH.

In one embodiment, X₁ and X₂ are O, Y₁ to Y₃ are N.

In one embodiment, X₁ and X₂ are O, Y₁ and Y₂ are N, and Y₃ is CH.

In one embodiment, one of X₁ and X₂ is S and the other is O, and Y₁ to Y₃ are N.

In one embodiment, one of X₁ and X₂ is S and the other is O, Y₁ and Y₂ are N, and Y₃ is CH.

Details of each substituent in the formulas (A1), (A2-1) to (A2-4), (A3), (A4-1), (A4-2), (A5-1), (A5-2), (A6), (A7), (1), (2), (2H), (3-1), (3-2), (3H-1), (3H-2), (4), (4H), and (5), and each substituent in the case of “a substituted or unsubstituted” are as defined in the [Definition] part of this specification.

Specific examples of the compound represented by the formula (A1) are described below, but these are merely examples, and the compound represented by the formula (A1) is not limited to the following specific examples. In the following specific examples, D represents a deuterium atom.

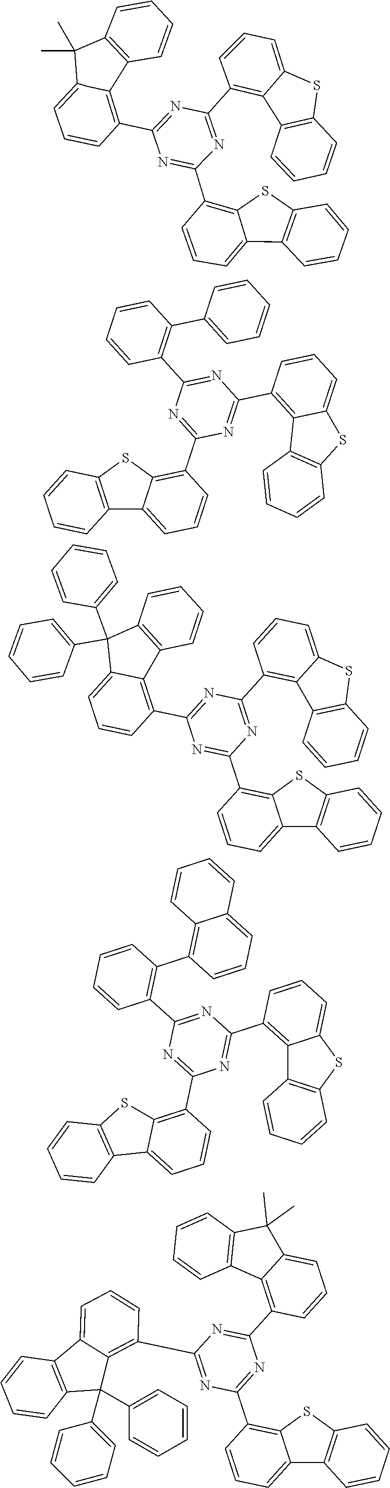

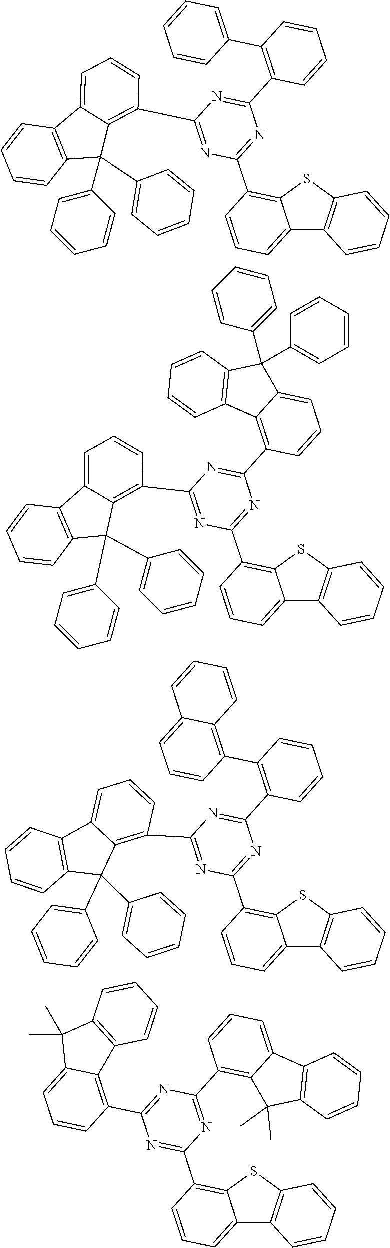

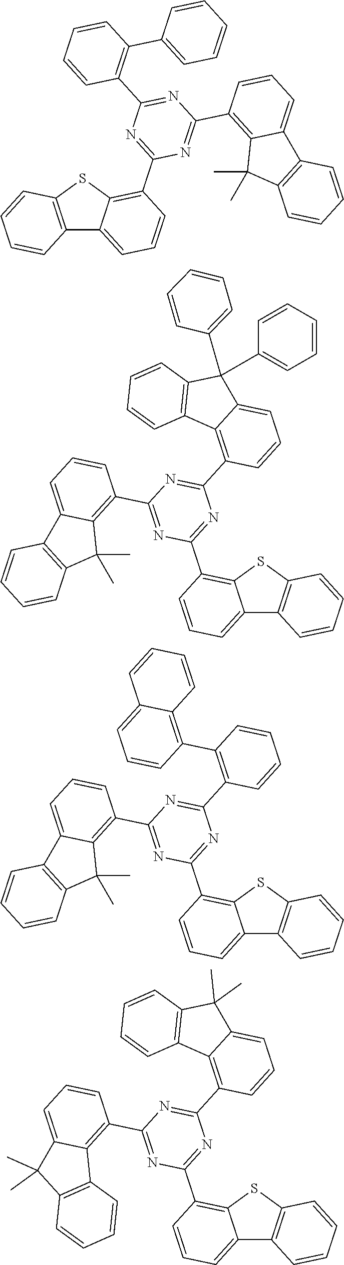

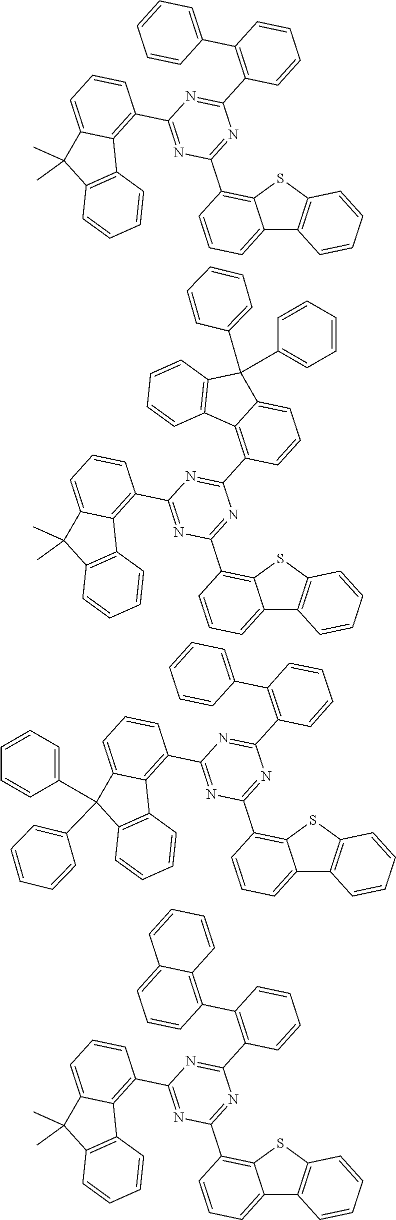

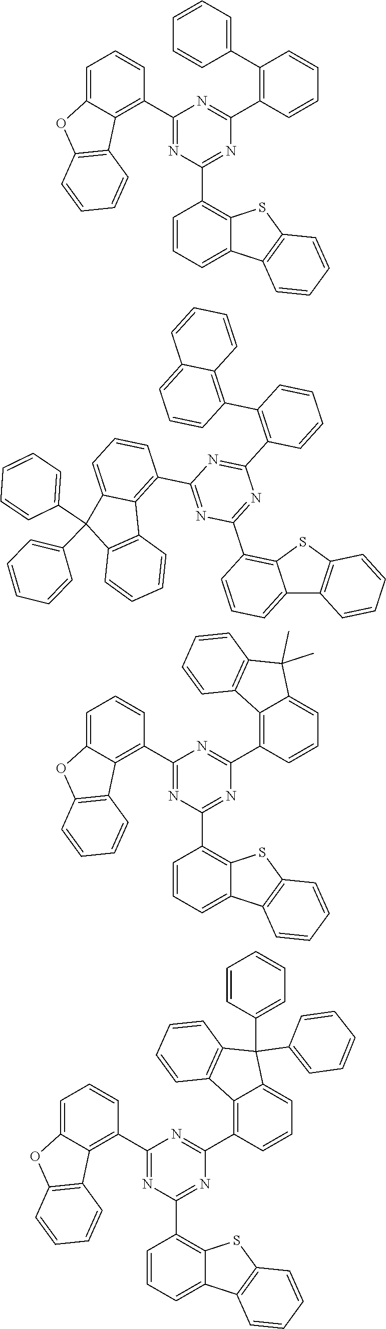

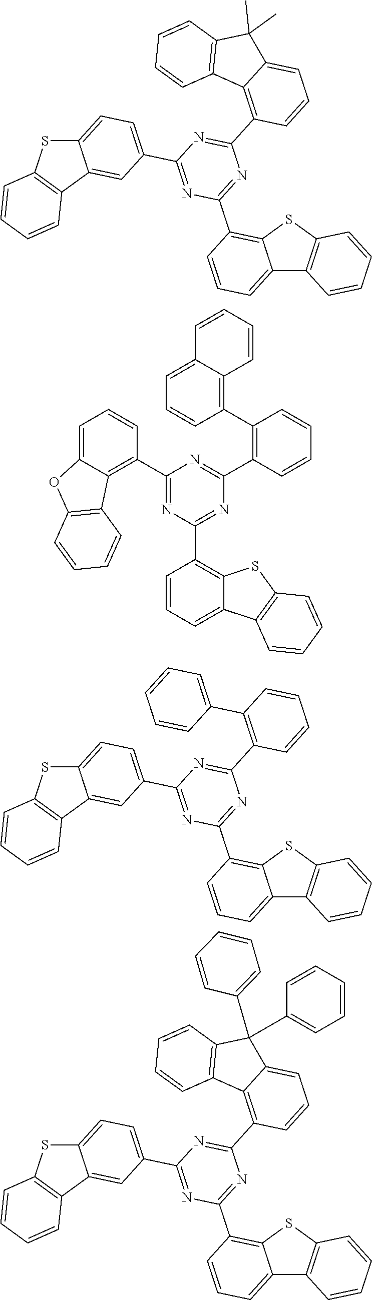

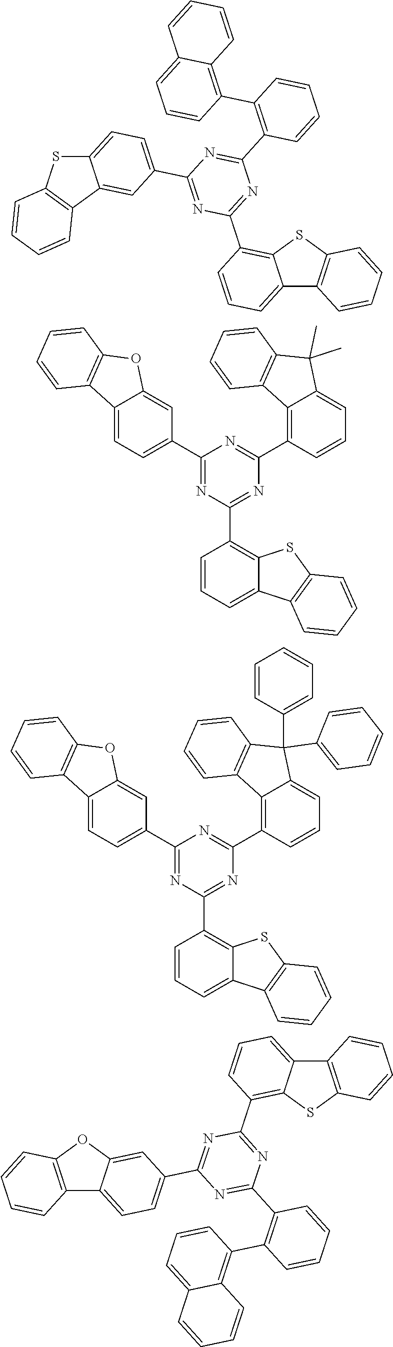

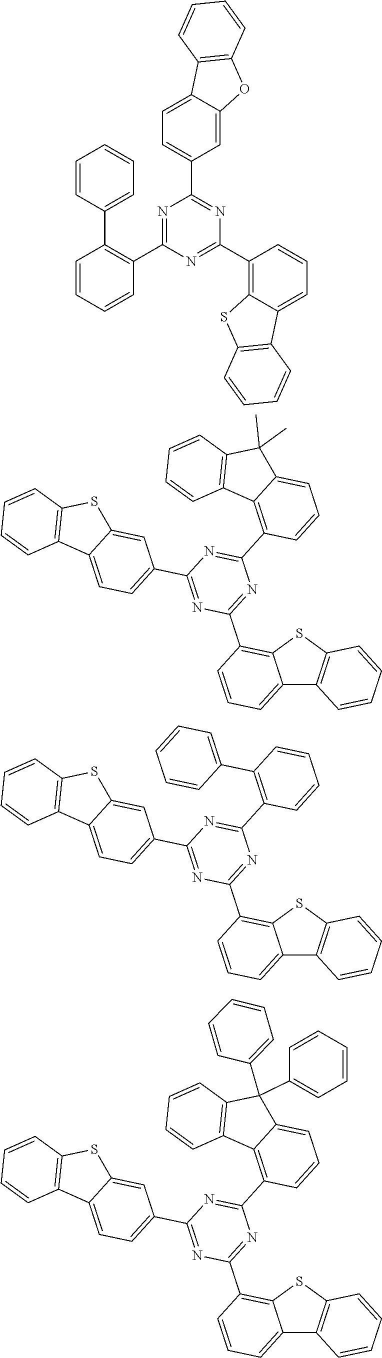

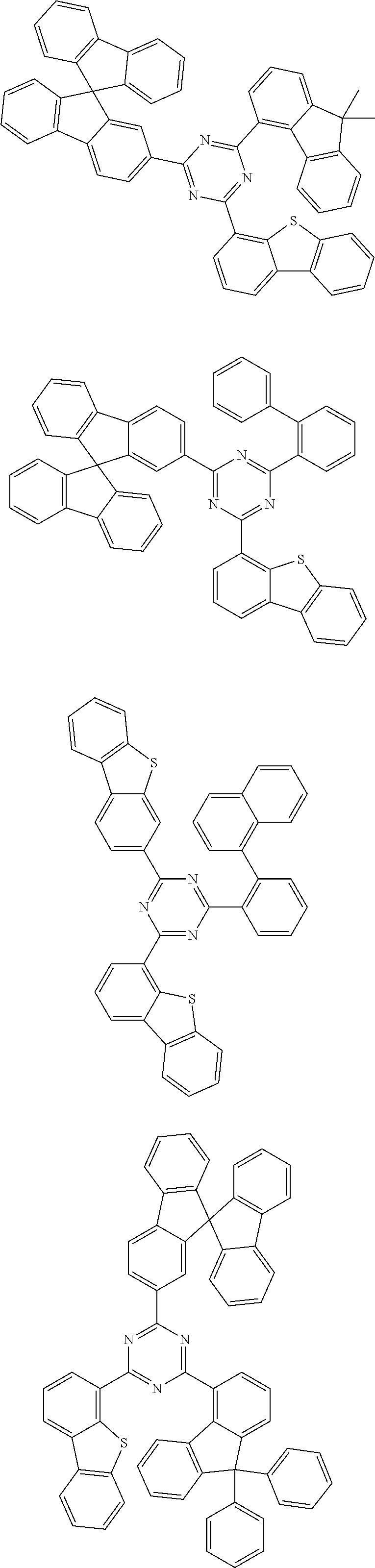

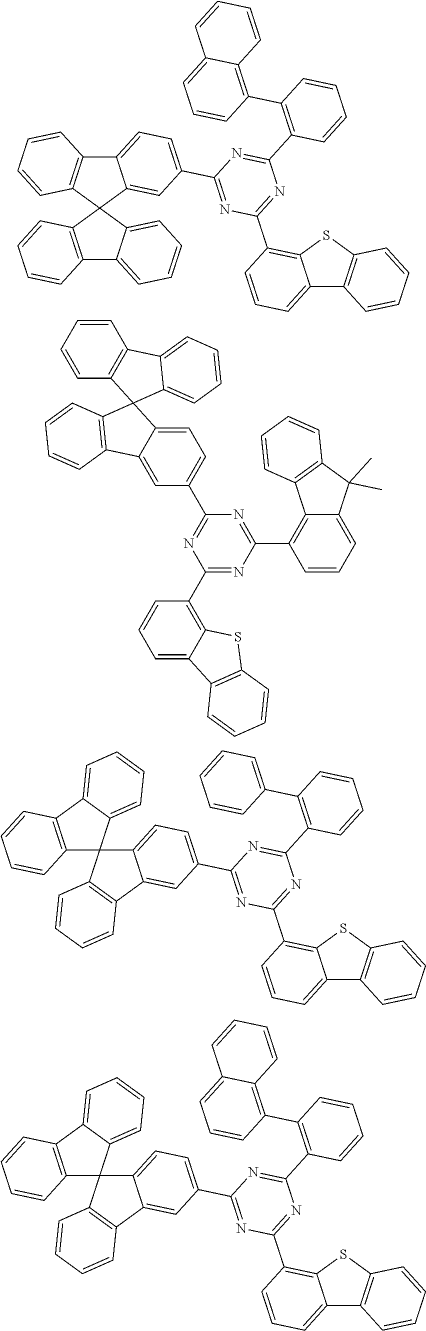

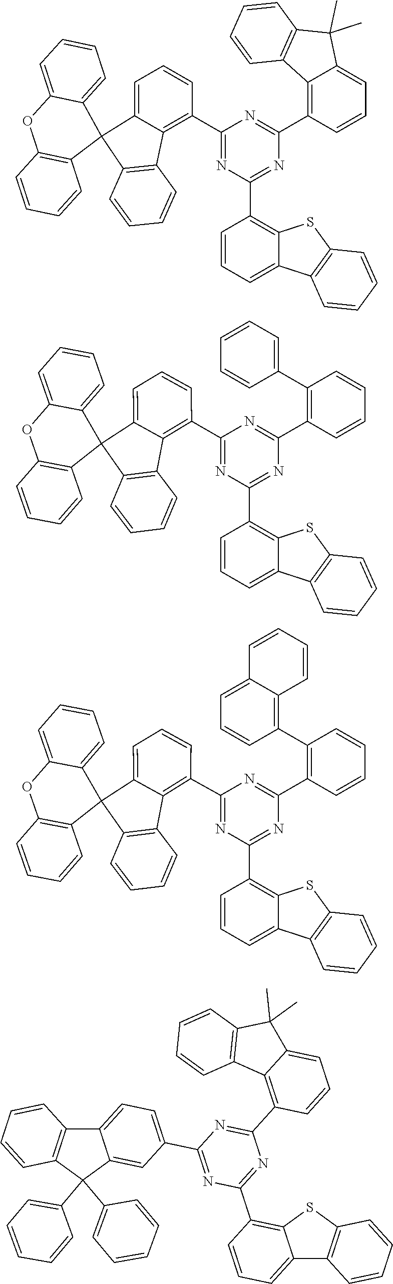

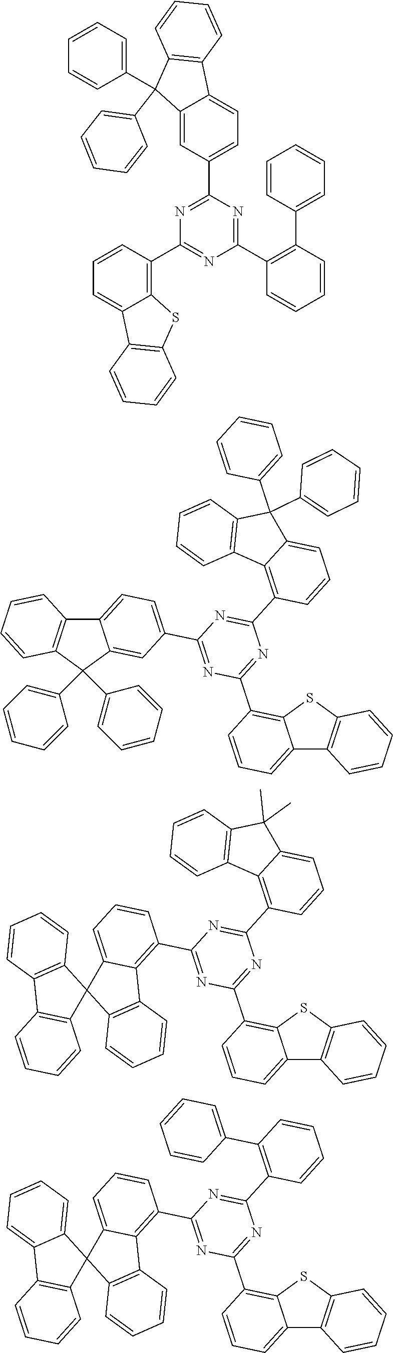

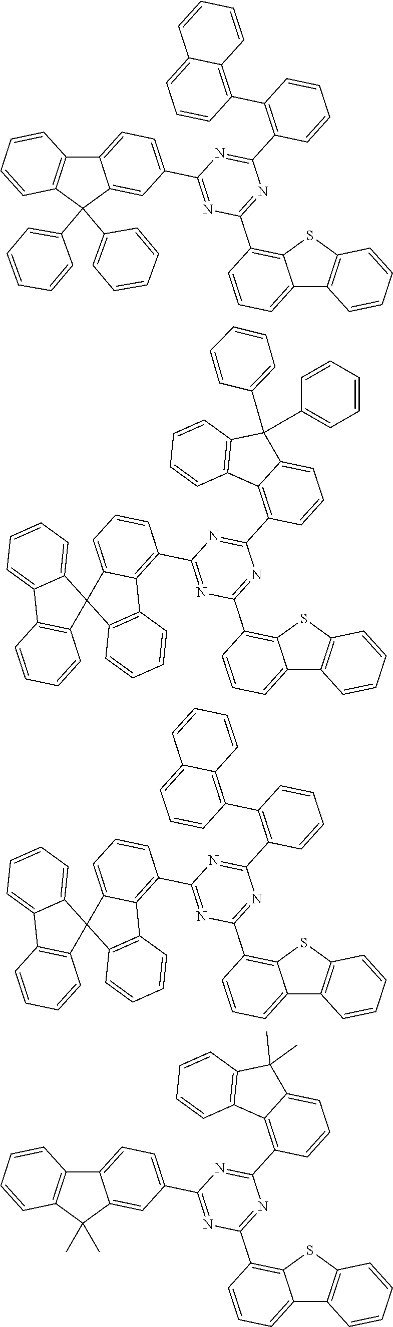

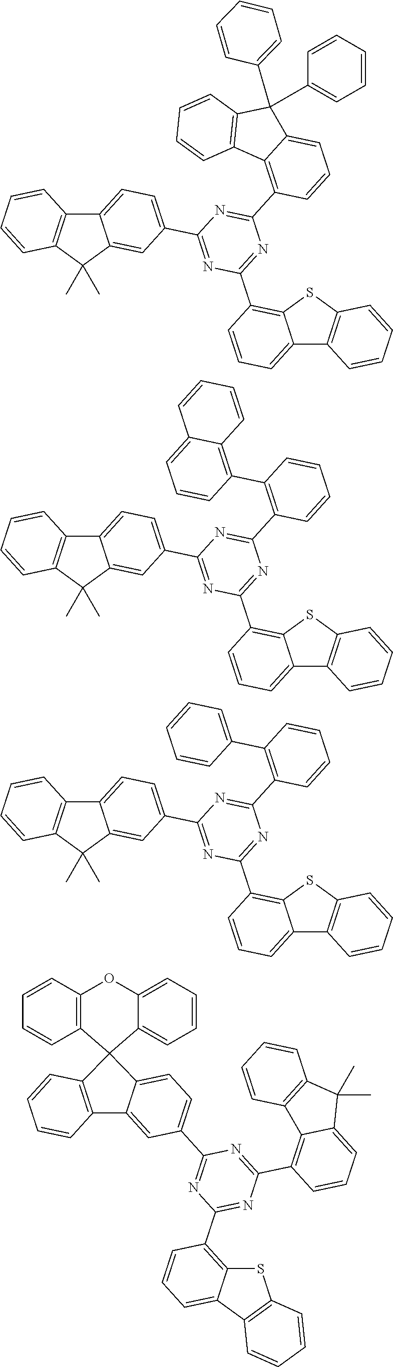

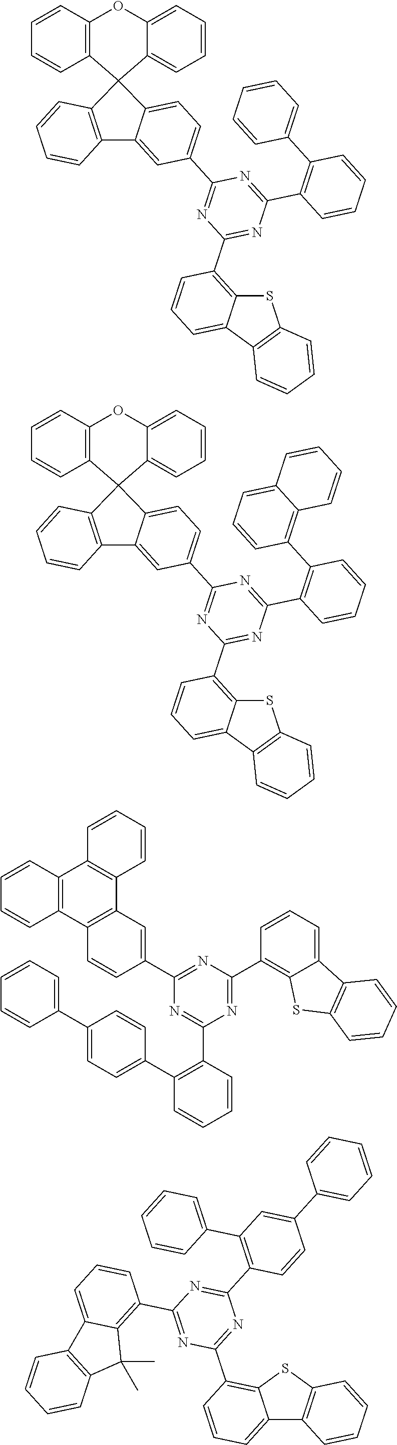 [SMILES: c1ccc(-c2ccccc2-c2nc(-c3ccc4c(c3)-c3ccccc3C43c4ccccc4Oc4ccccc43)nc(-c3cccc4c3sc3ccccc34)n2)cc1 ; c1ccc2c(c1)Oc1ccccc1C21c2ccccc2-c2cc(-c3nc(-c4ccccc4-c4cccc5ccccc45)nc(-c4cccc5c4sc4ccccc45)n3)ccc21 ; c1ccc(-c2ccc(-c3ccccc3-c3nc(-c4ccc5c6ccccc6c6ccccc6c5c4)nc(-c4cccc5c4sc4ccccc45)n3)cc2)cc1 ; CC1(C)c2ccccc2-c2cccc(-c3nc(-c4ccc(-c5ccccc5)cc4-c4ccccc4)nc(-c4cccc5c4sc4ccccc45)n3)c21]

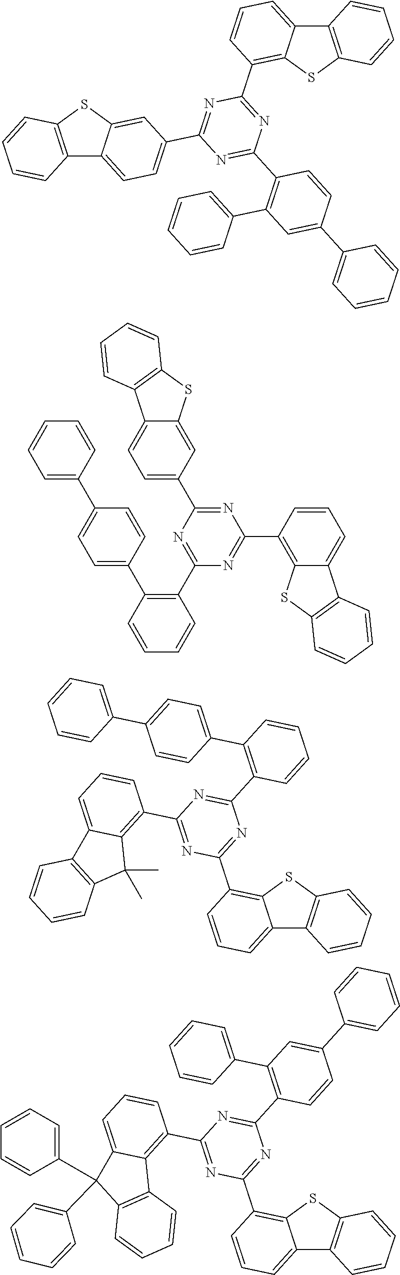

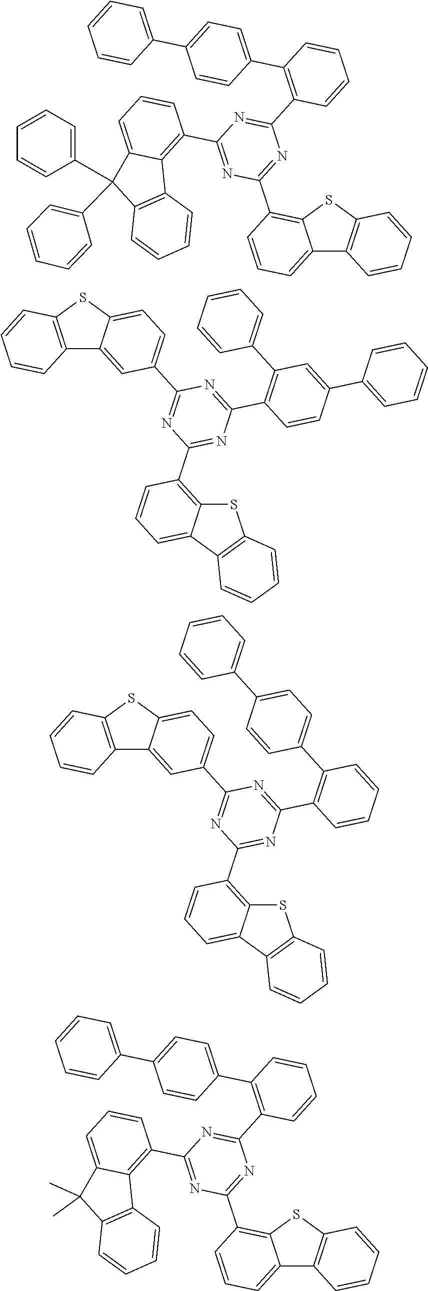

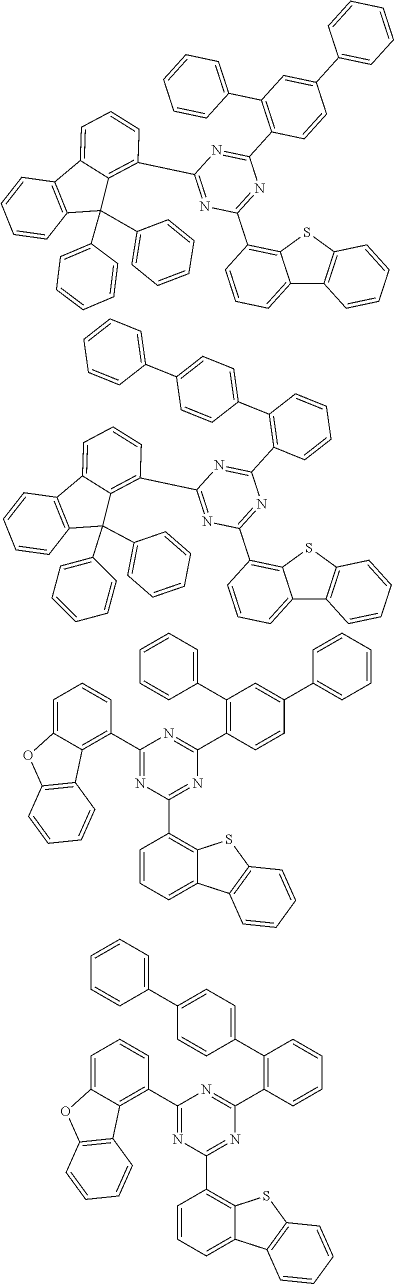

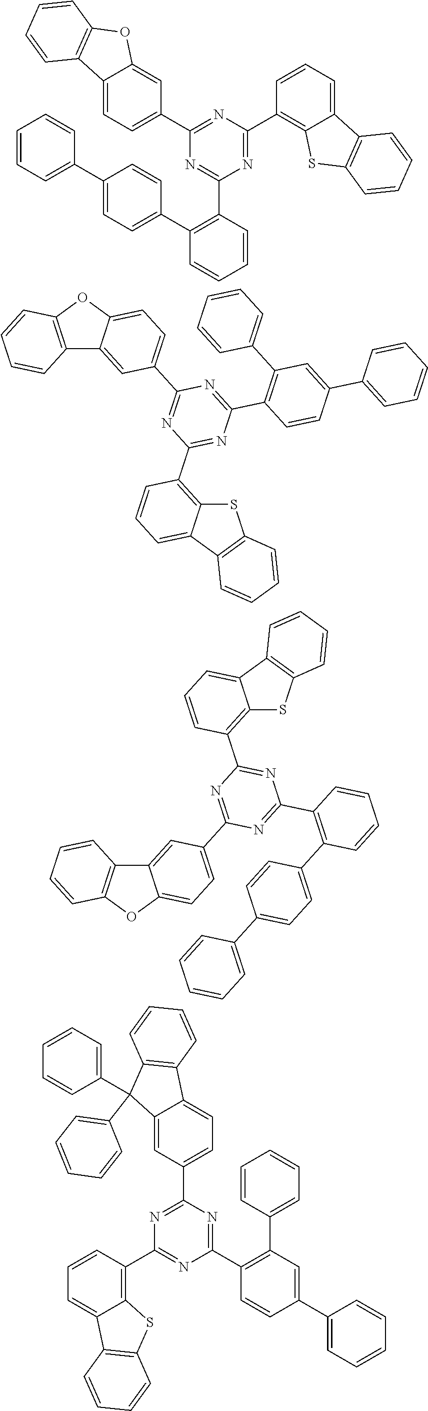

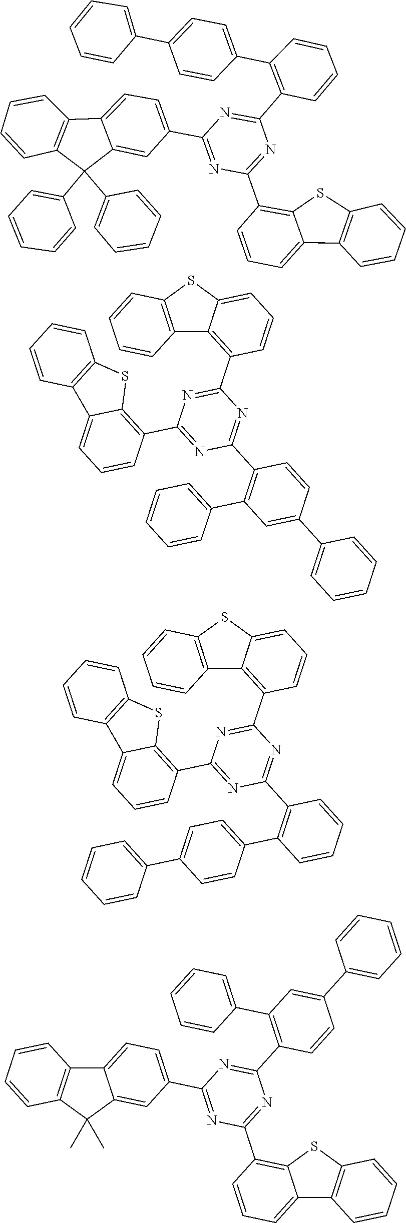

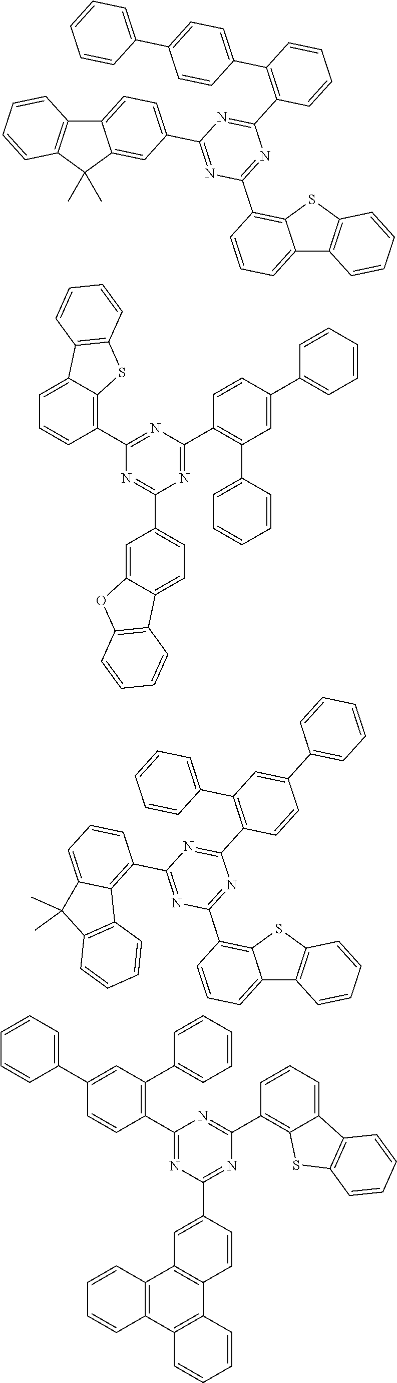

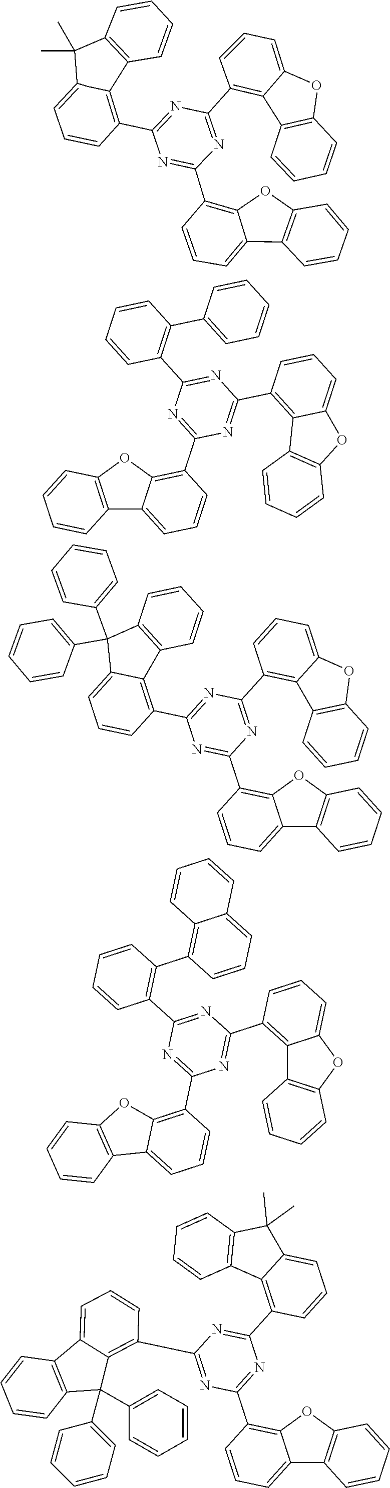

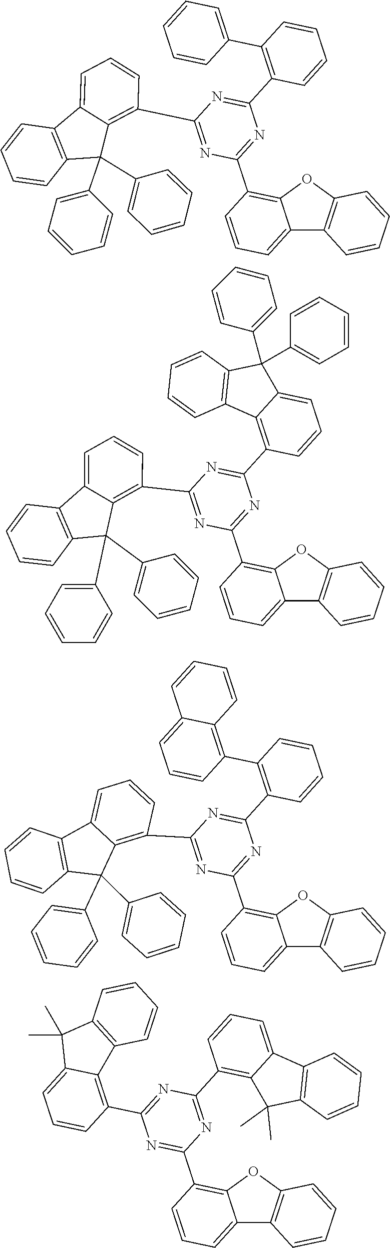

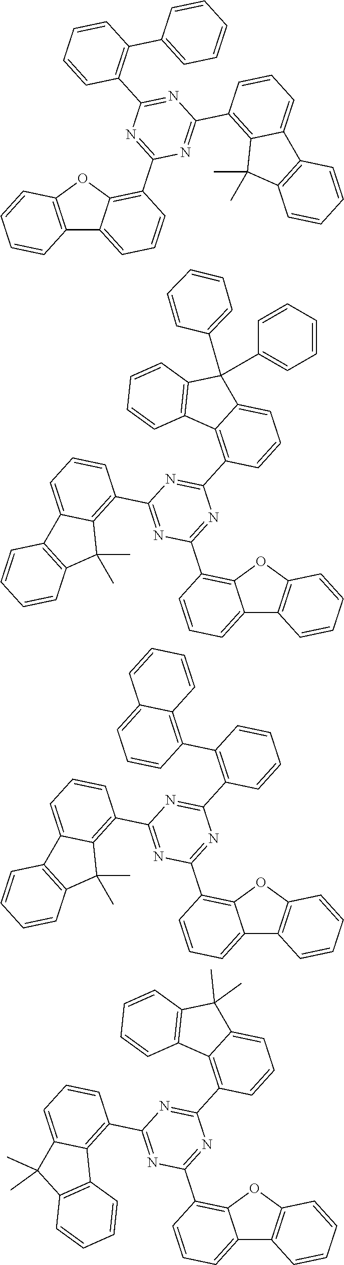

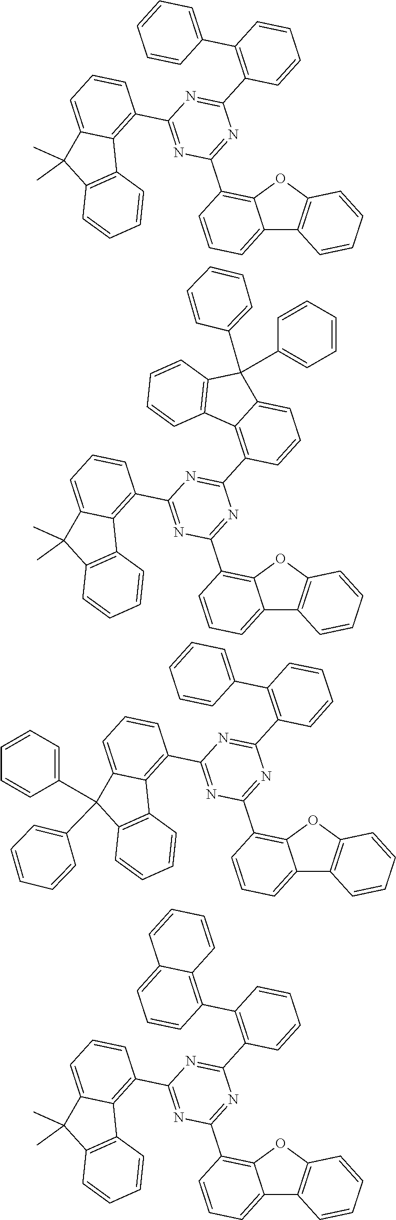

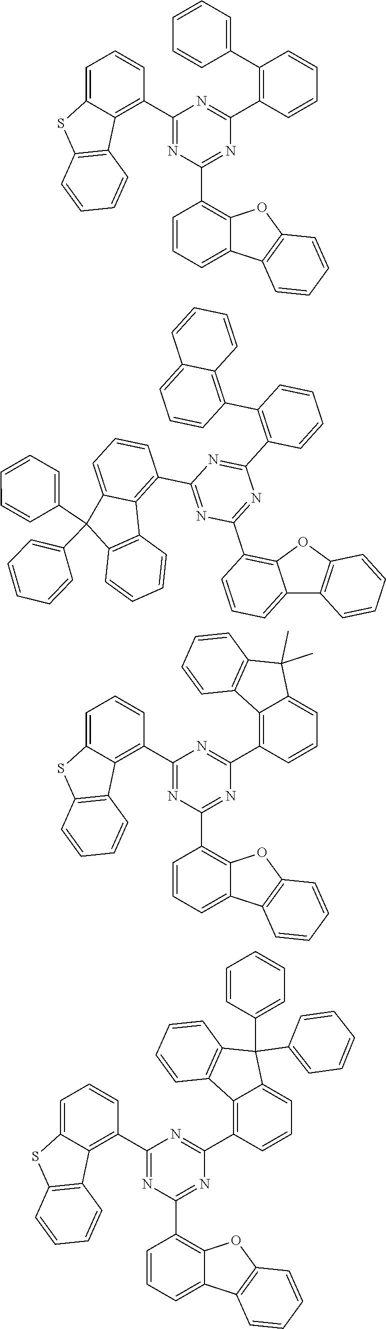

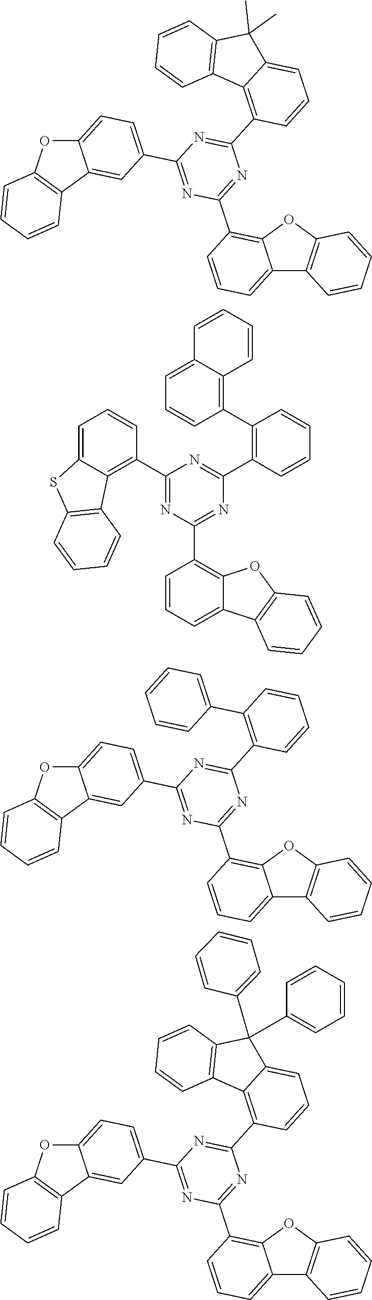

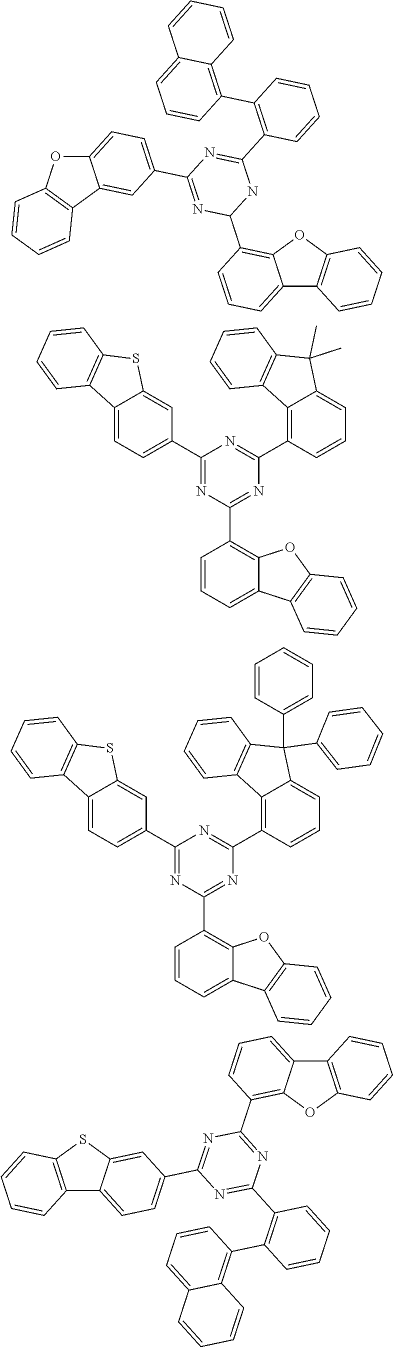

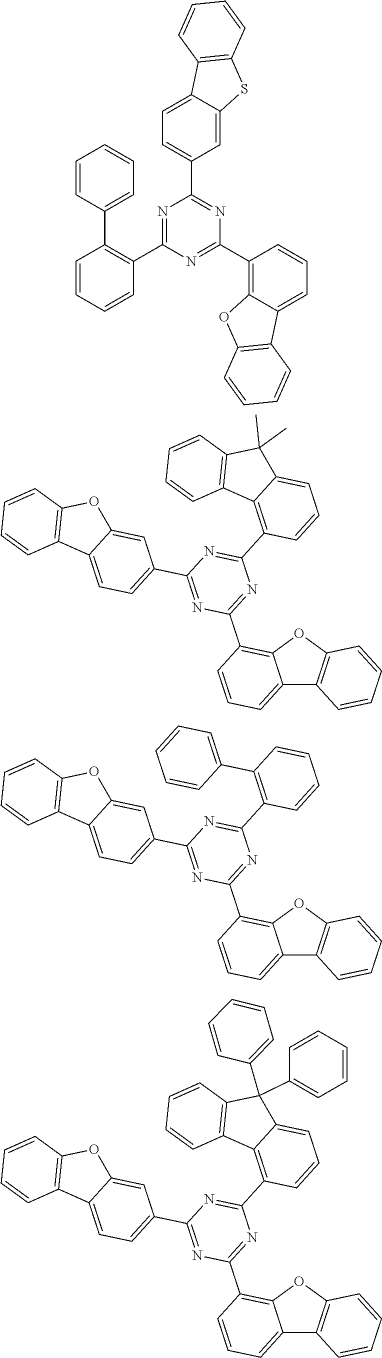

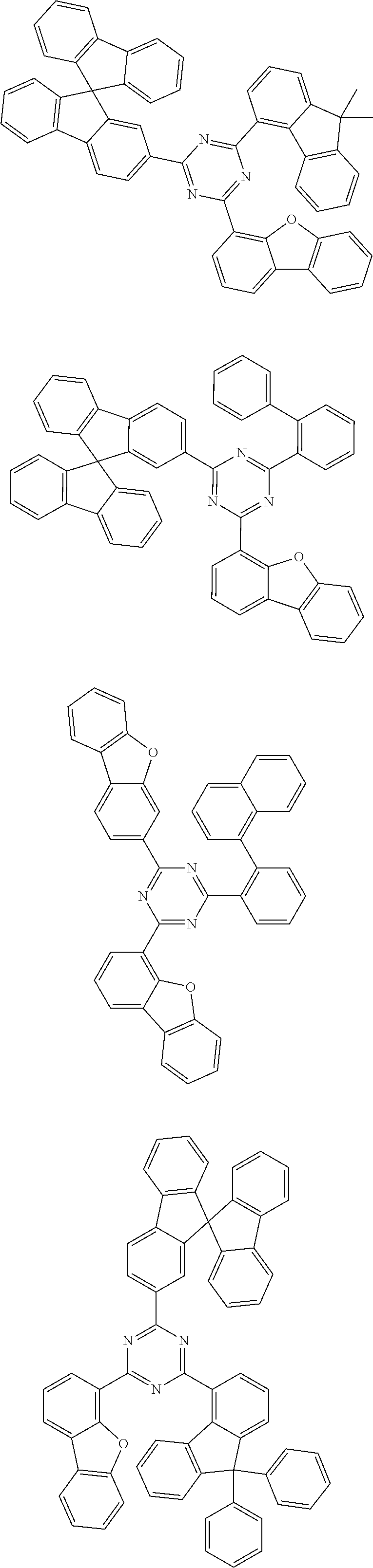 [SMILES: CC1(C)c2ccccc2-c2c(-c3nc(-c4ccc5c(c4)C4(c6ccccc6-c6ccccc64)c4ccccc4-5)nc(-c4cccc5c4oc4ccccc45)n3)cccc21 ; c1ccc(-c2ccccc2-c2nc(-c3ccc4c(c3)C3(c5ccccc5-c5ccccc53)c3ccccc3-4)nc(-c3cccc4c3oc3ccccc34)n2)cc1 ; c1ccc(-c2cccc3ccccc23)c(-c2nc(-c3ccc4c(c3)oc3ccccc34)nc(-c3cccc4c3oc3ccccc34)n2)c1 ; c1ccc(C2(c3ccccc3)c3ccccc3-c3c(-c4nc(-c5ccc6c(c5)C5(c7ccccc7-c7ccccc75)c5ccccc5-6)nc(-c5cccc6c5oc5ccccc56)n4)cccc32)cc1]

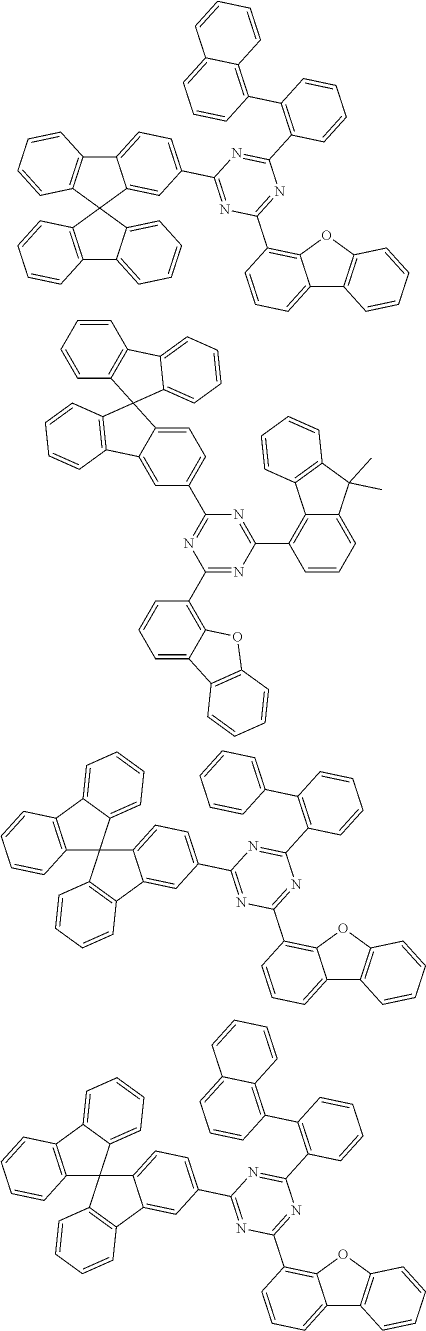

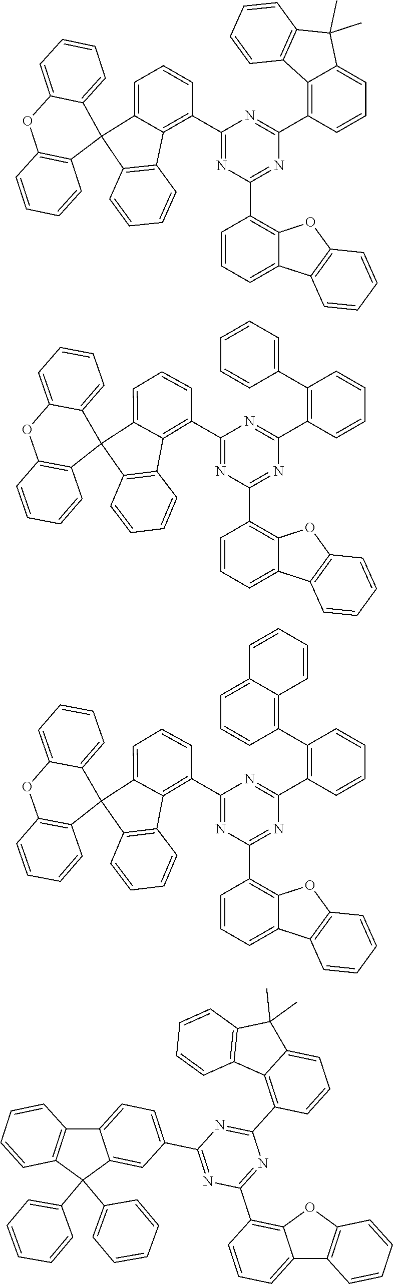

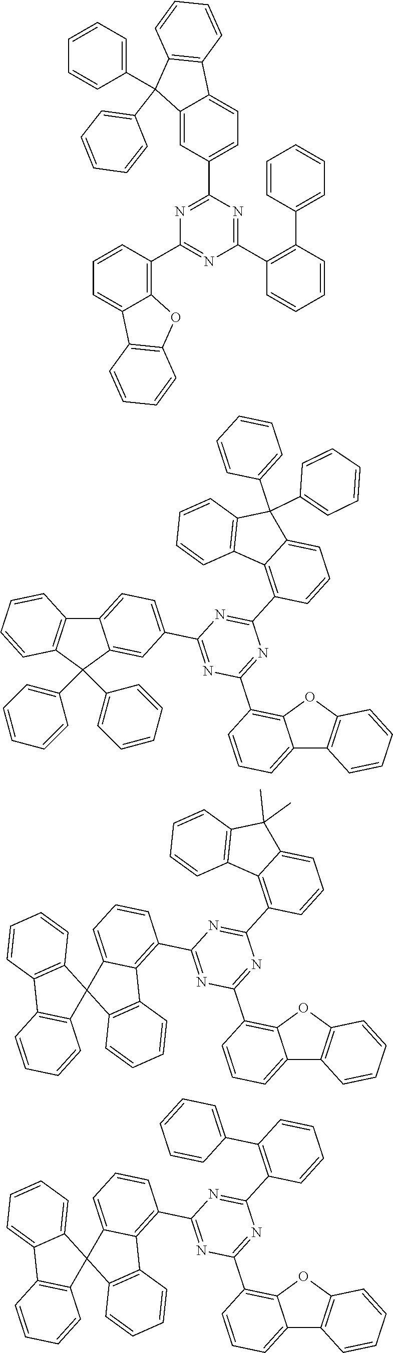

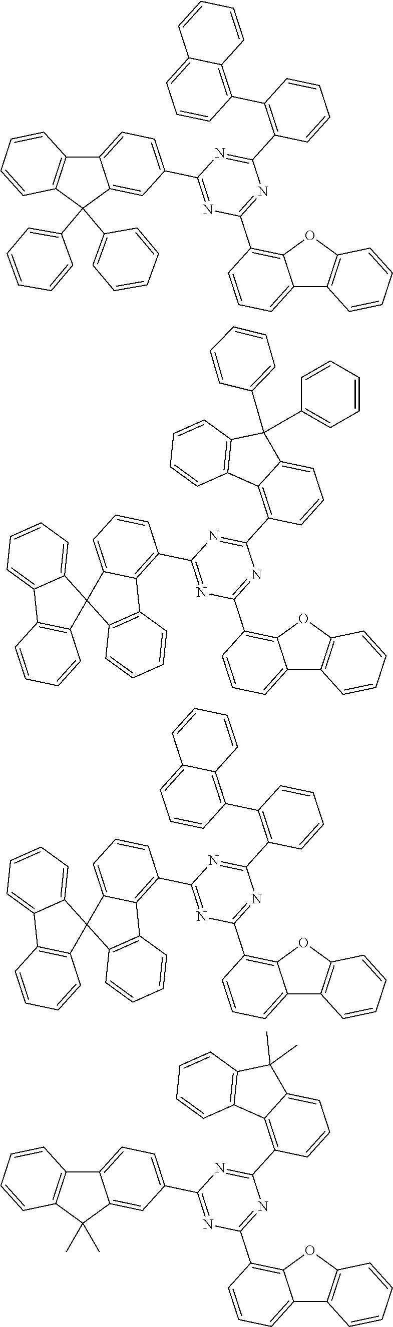

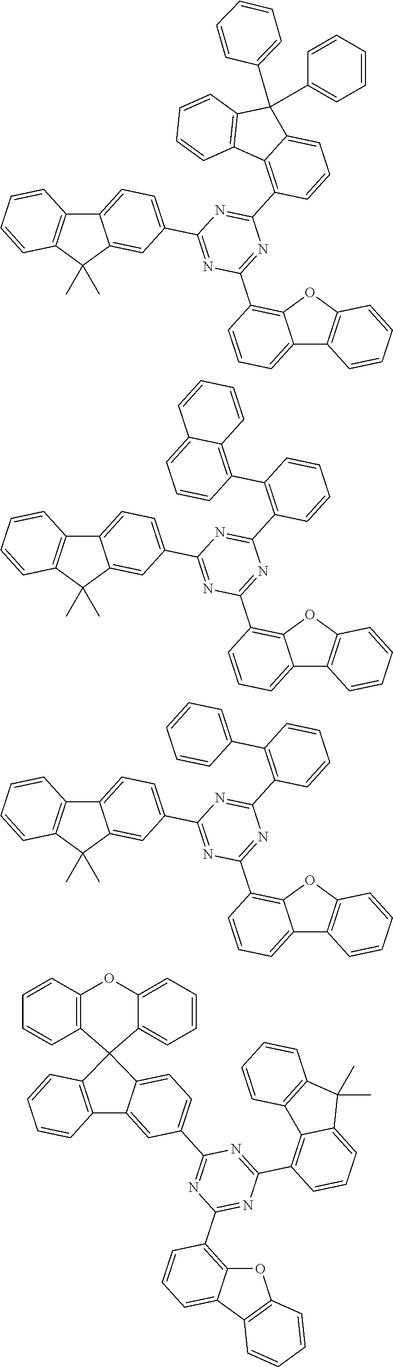

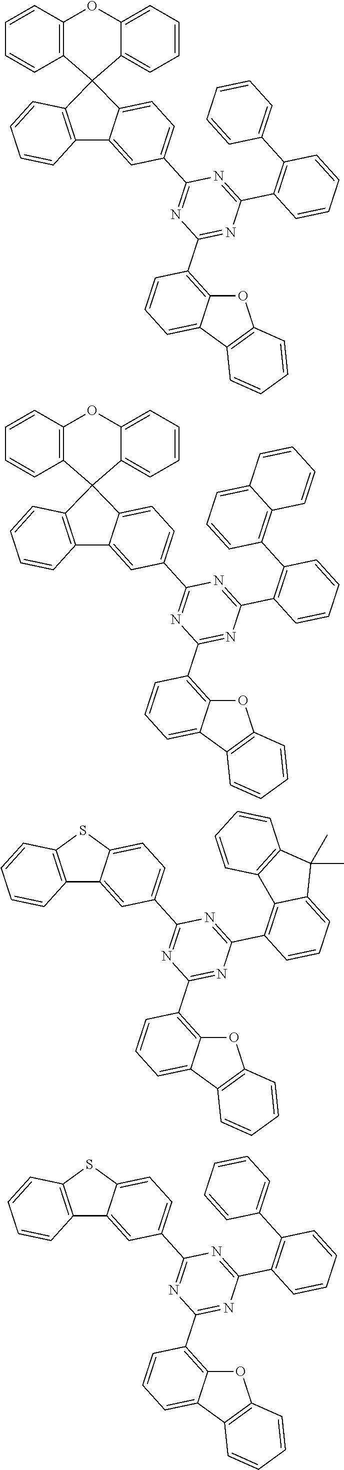

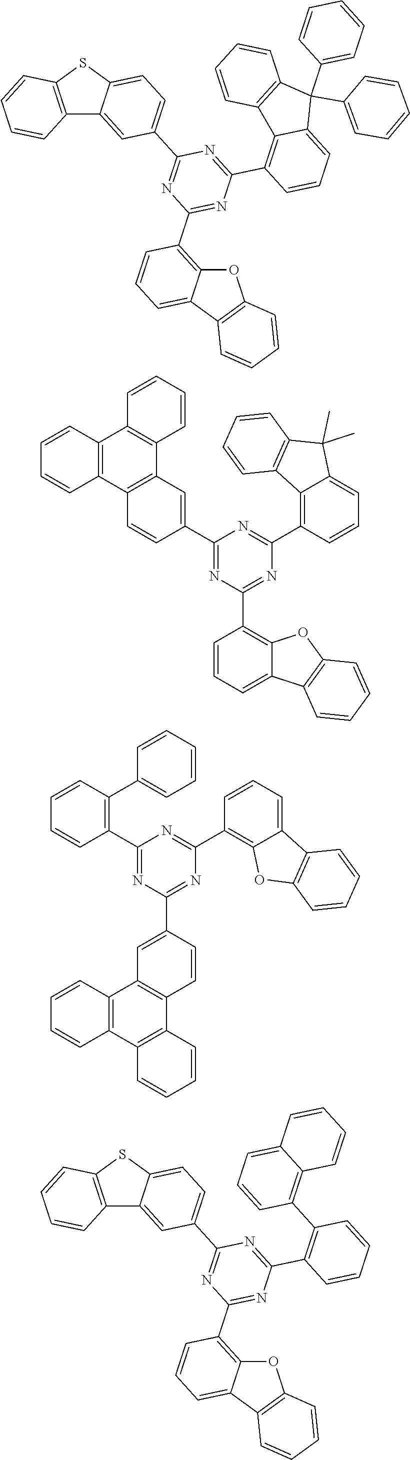 [SMILES: c1ccc(C2(c3ccccc3)c3ccccc3-c3c(-c4nc(-c5ccc6sc7ccccc7c6c5)nc(-c5cccc6c5oc5ccccc56)n4)cccc32)cc1 ; CC1(C)c2ccccc2-c2c(-c3nc(-c4ccc5c6ccccc6c6ccccc6c5c4)nc(-c4cccc5c4oc4ccccc45)n3)cccc21 ; c1ccc(-c2ccccc2-c2nc(-c3ccc4c5ccccc5c5ccccc5c4c3)nc(-c3cccc4c3oc3ccccc34)n2)cc1 ; c1ccc(-c2cccc3ccccc23)c(-c2nc(-c3ccc4sc5ccccc5c4c3)nc(-c3cccc4c3oc3ccccc34)n2)c1]

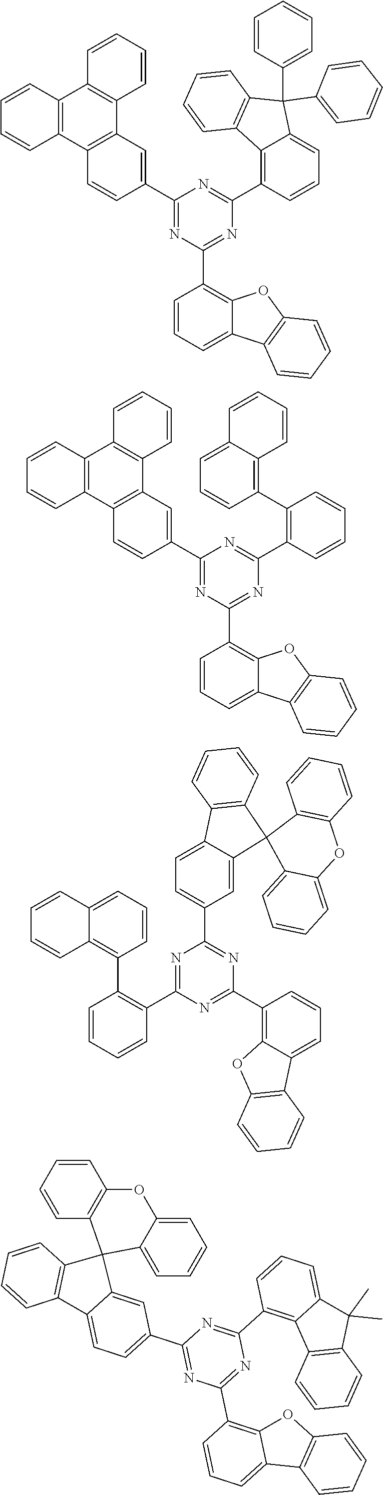

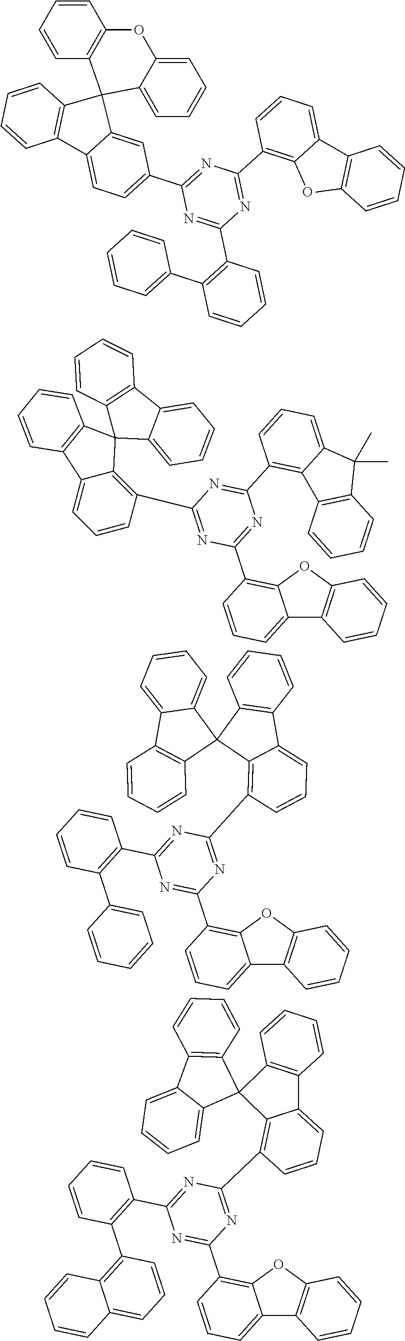

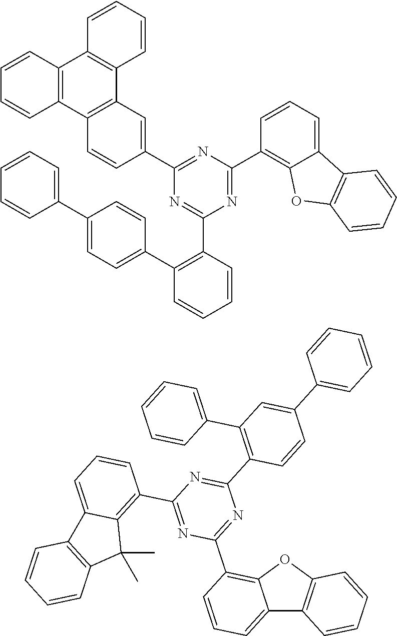 [SMILES: c1ccc(-c2ccc(-c3ccccc3-c3nc(-c4ccc5c6ccccc6c6ccccc6c5c4)nc(-c4cccc5c4oc4ccccc45)n3)cc2)cc1 ; CC1(C)c2ccccc2-c2cccc(-c3nc(-c4ccc(-c5ccccc5)cc4-c4ccccc4)nc(-c4cccc5c4oc4ccccc45)n3)c21]

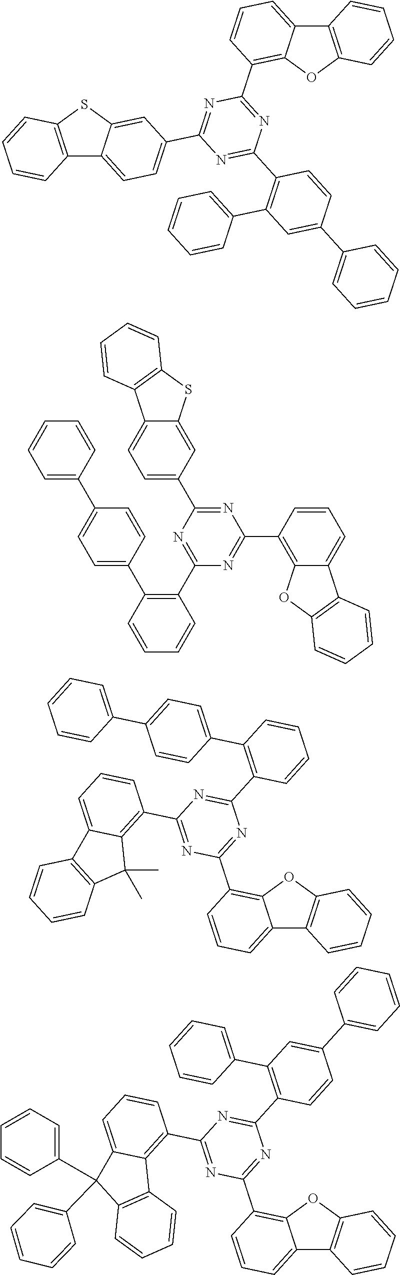

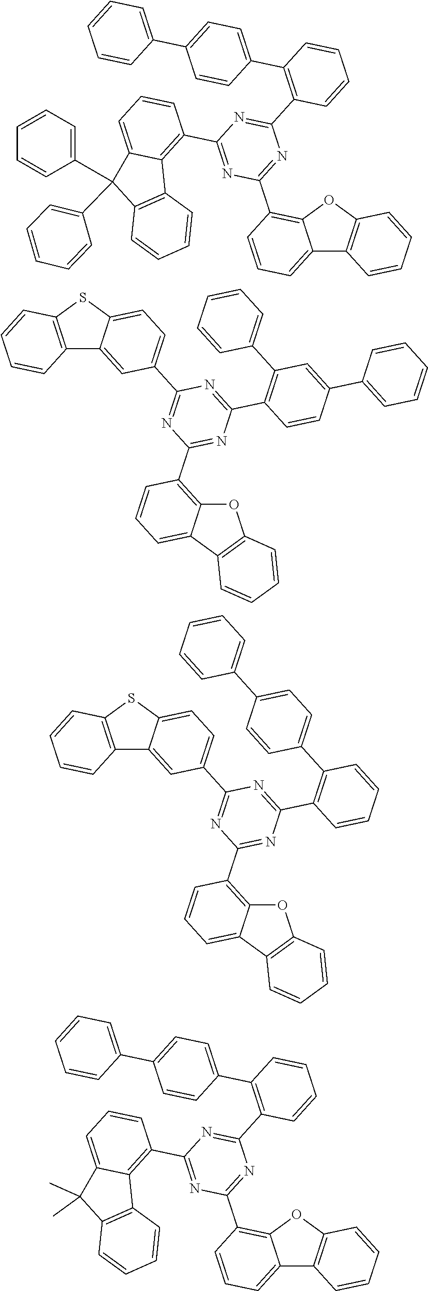

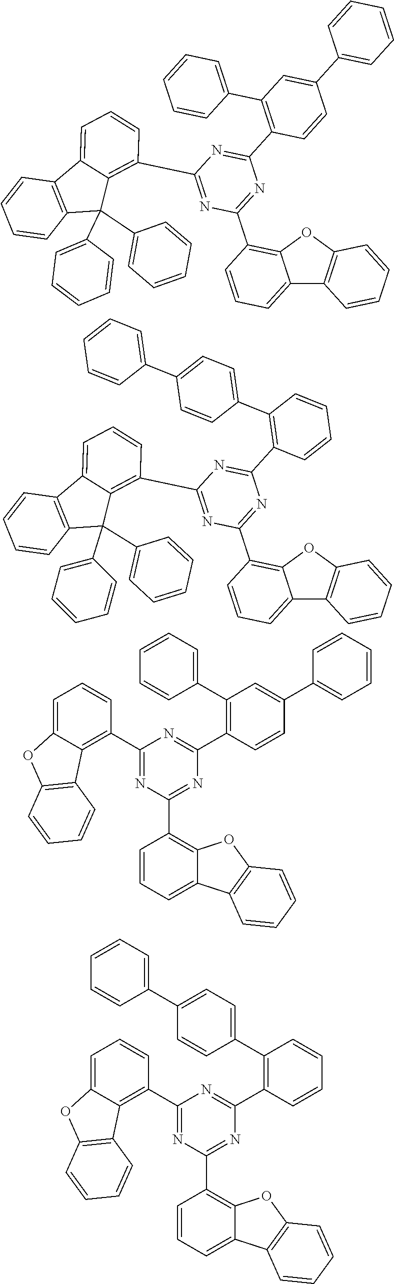

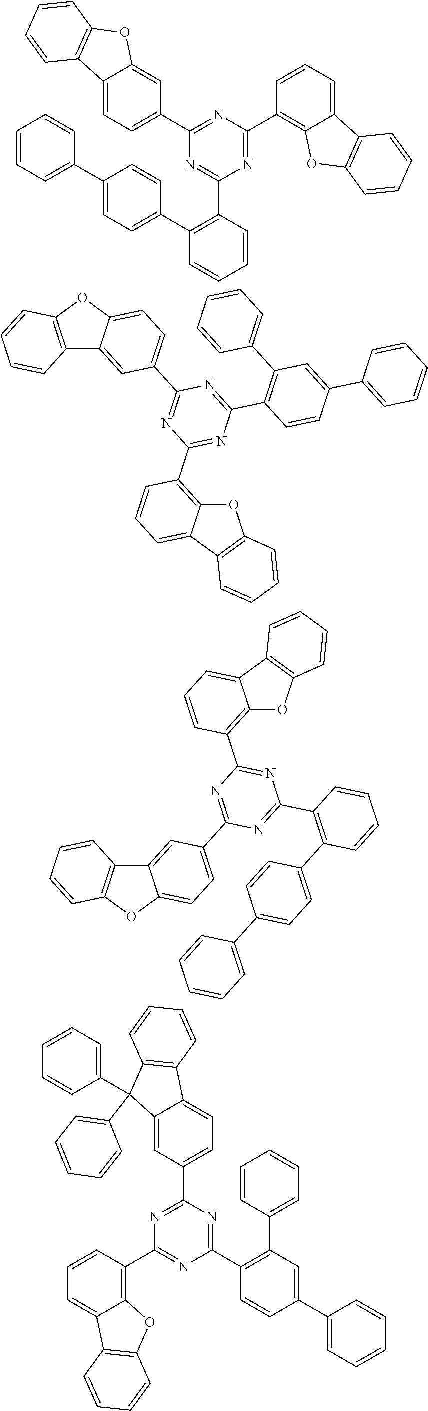

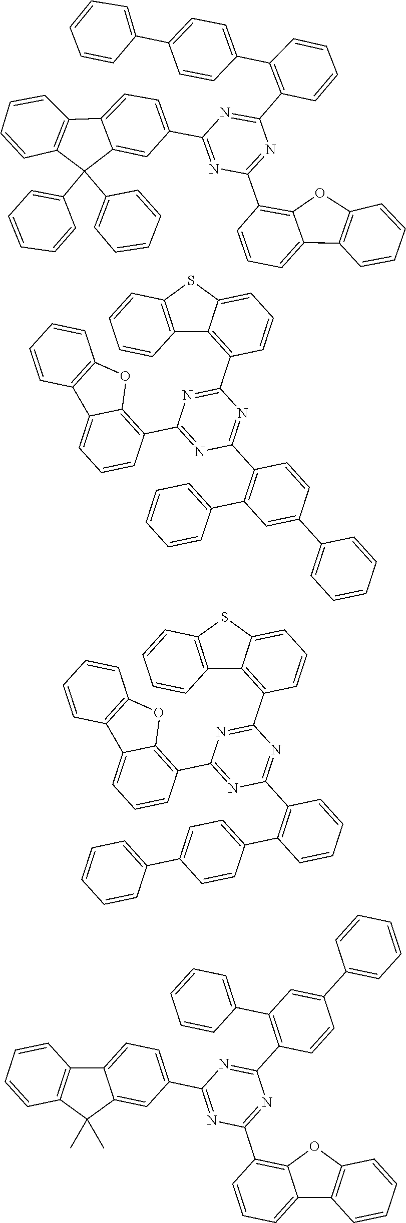

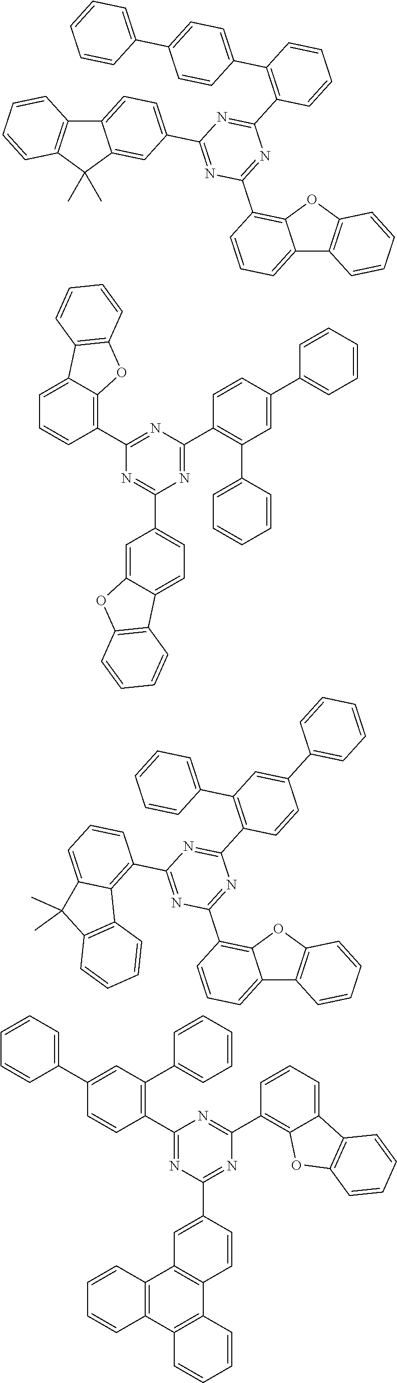

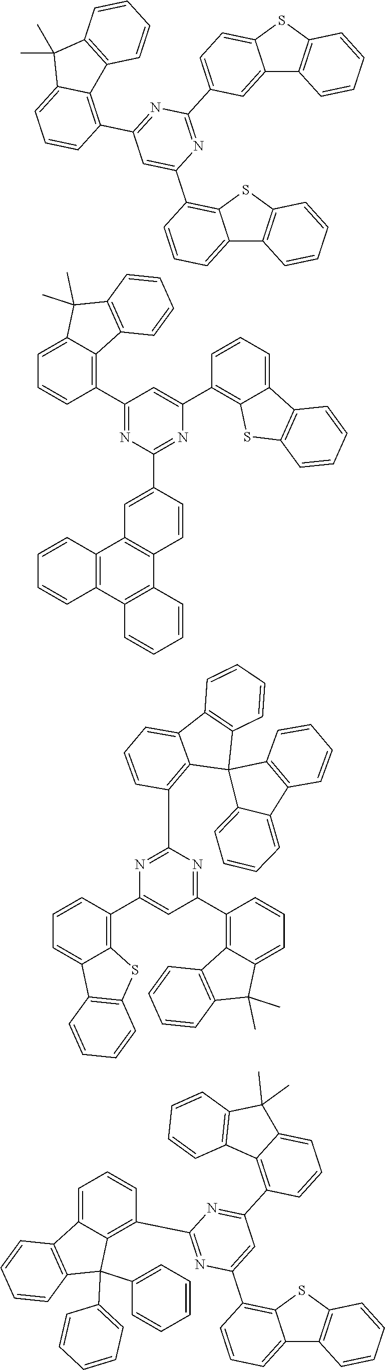

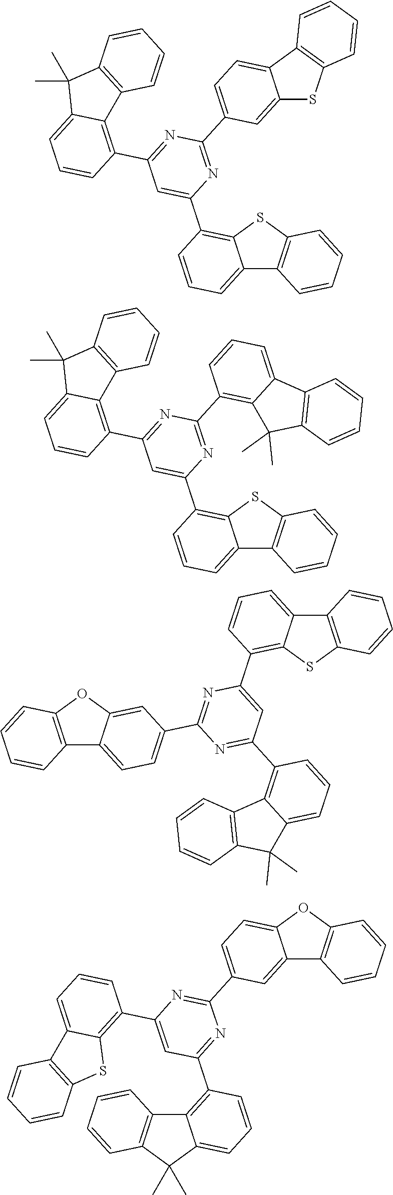

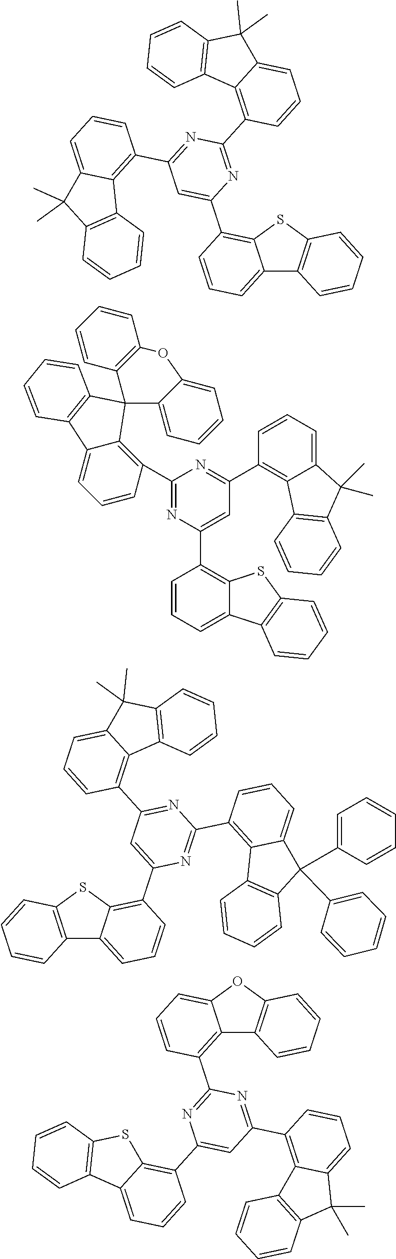

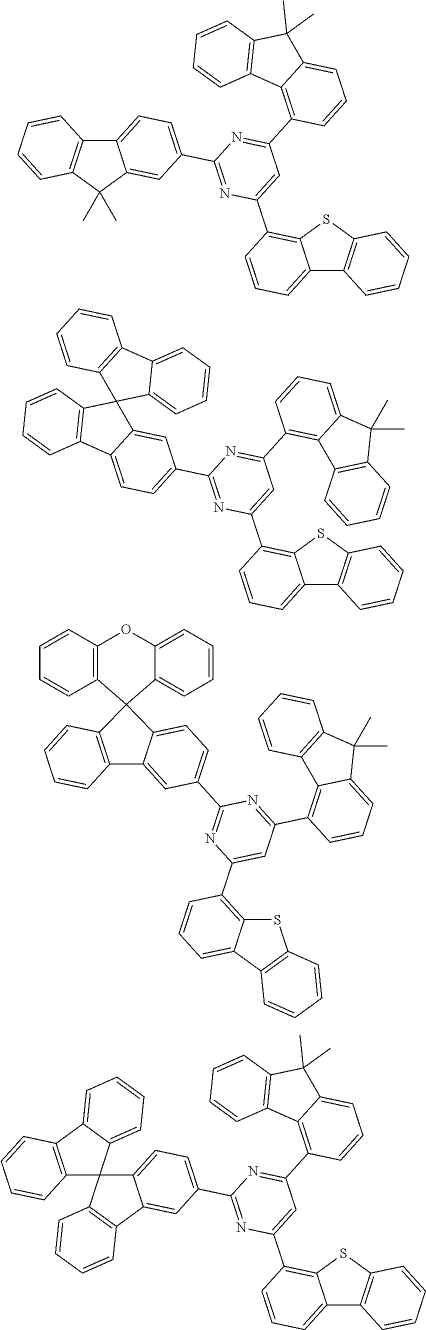

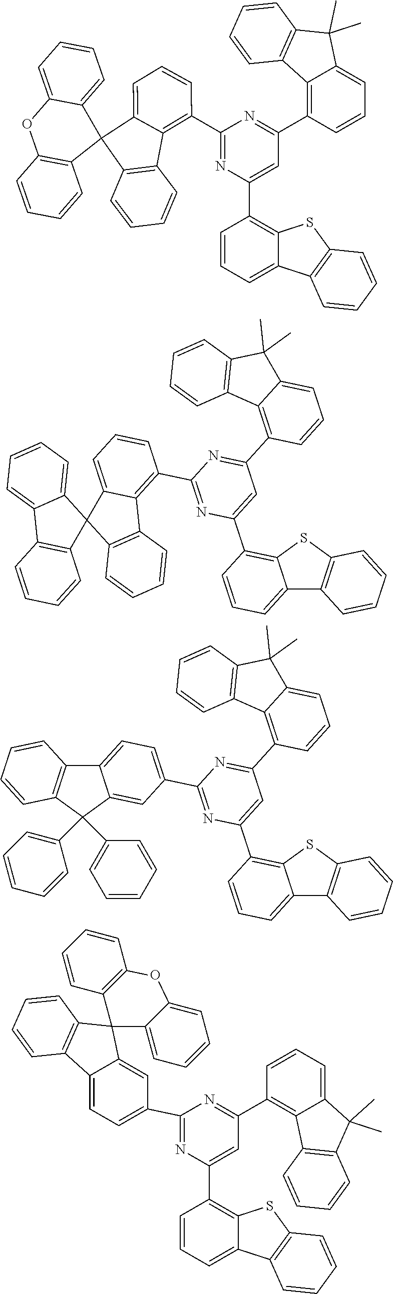

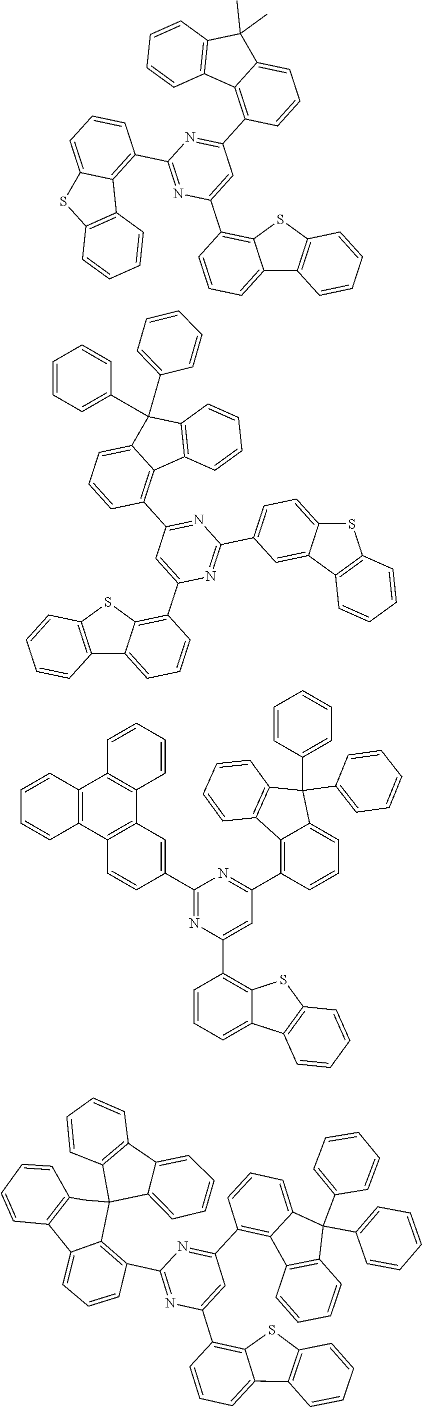

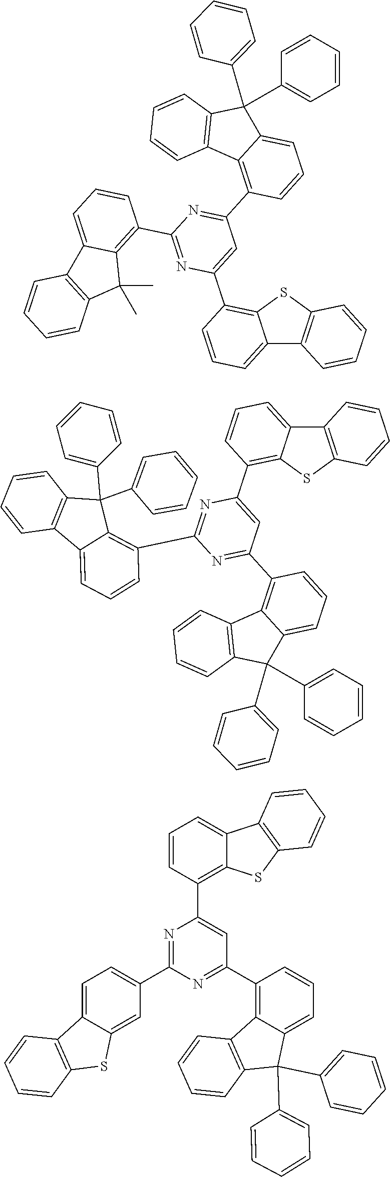

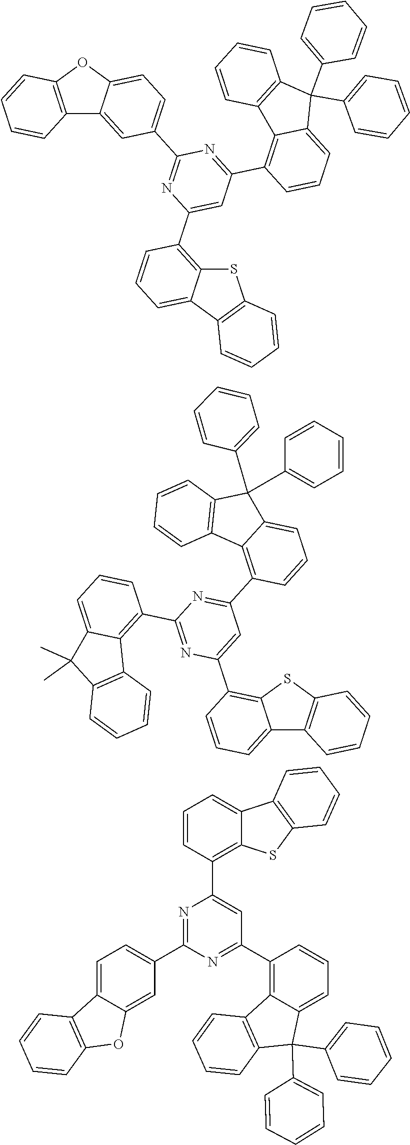

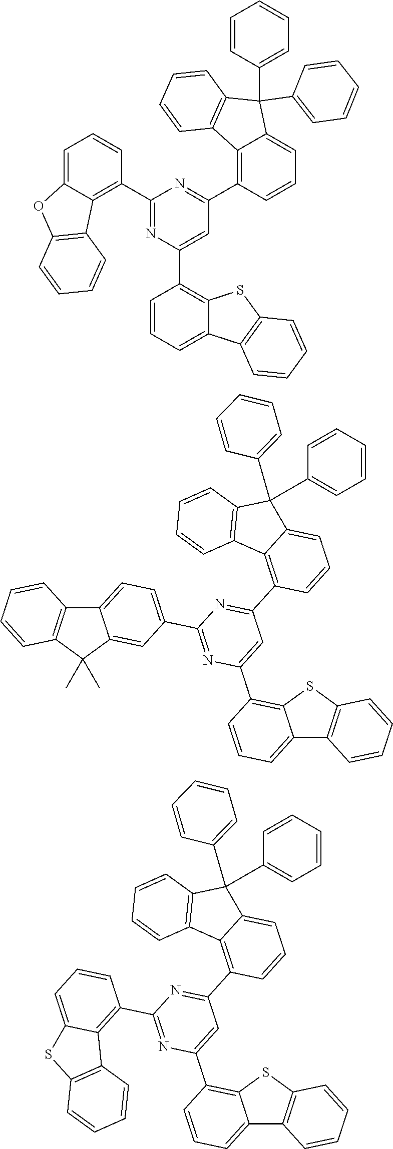

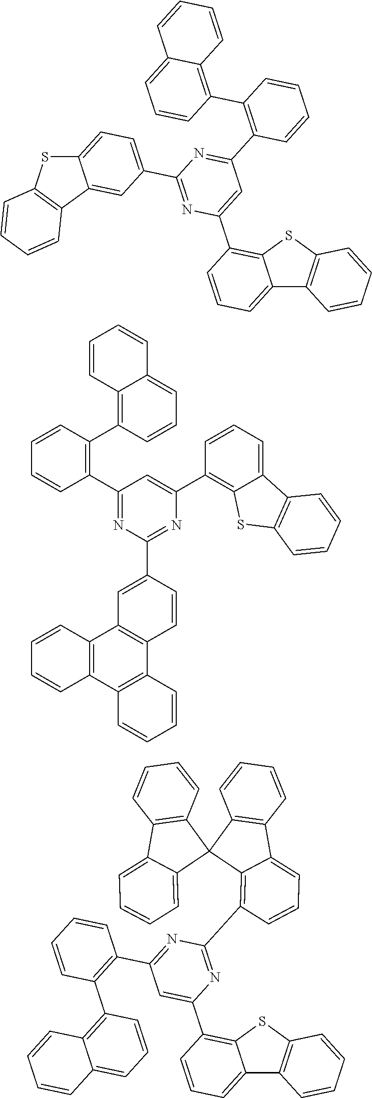

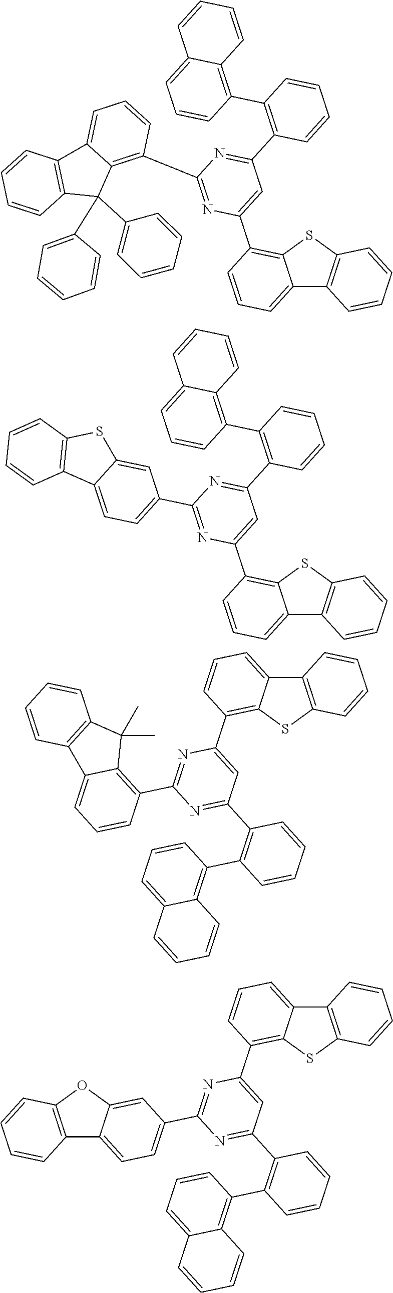

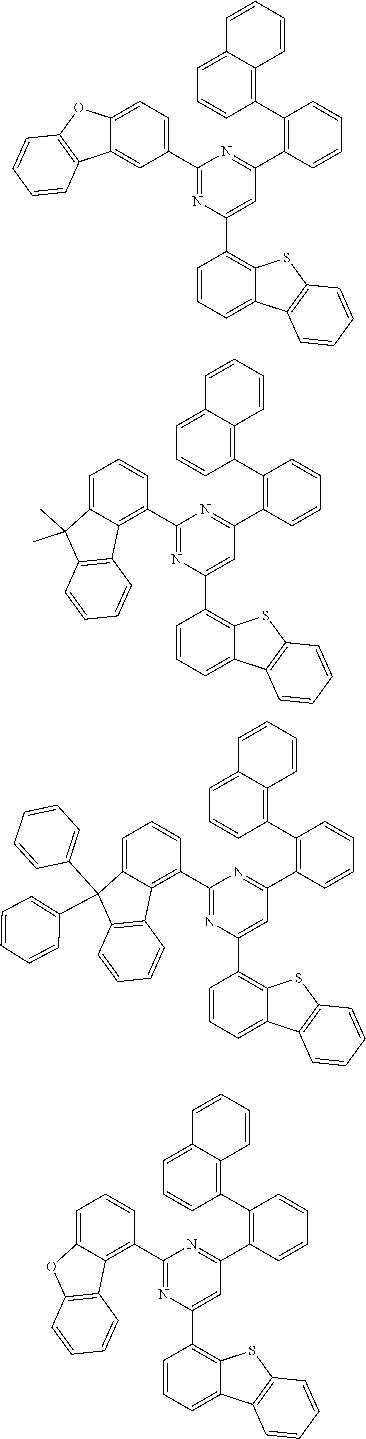

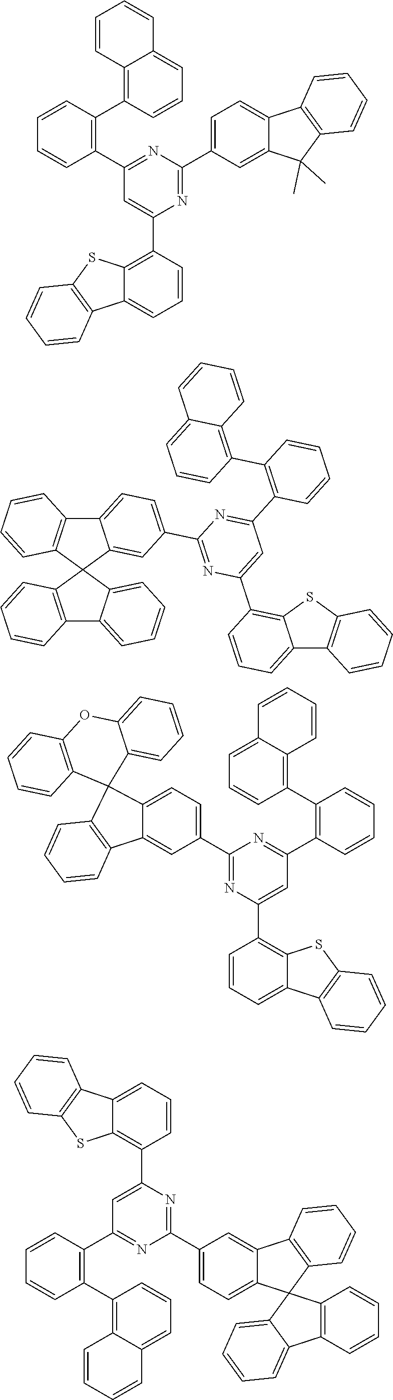

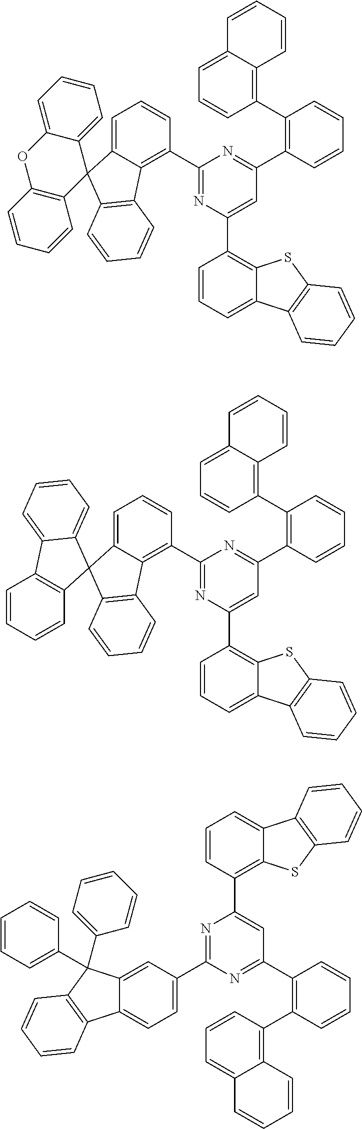

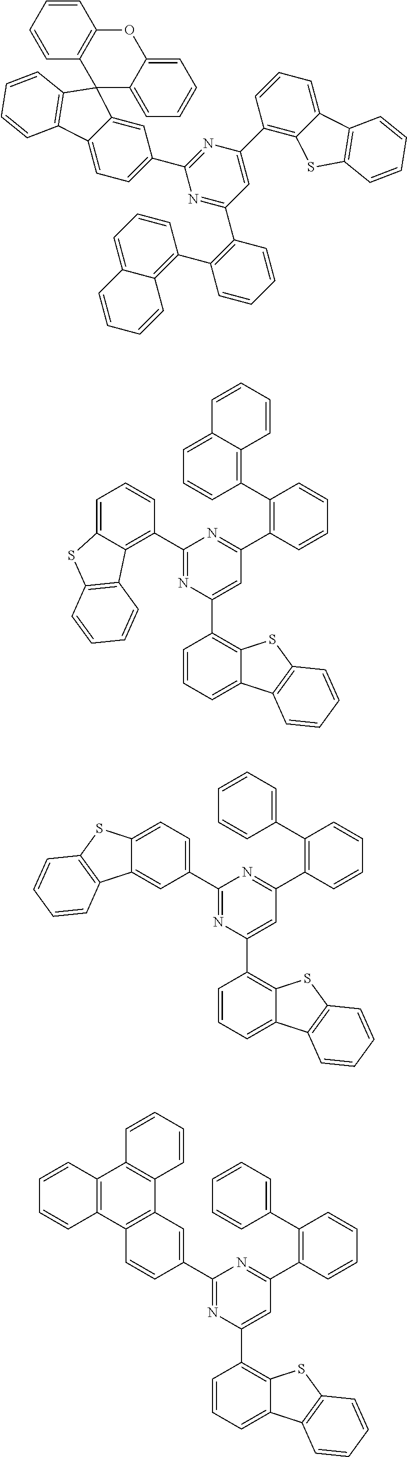 [SMILES: c1ccc2c(c1)Oc1ccccc1C21c2ccccc2-c2ccc(-c3nc(-c4ccccc4-c4cccc5ccccc45)cc(-c4cccc5c4sc4ccccc45)n3)cc21 ; c1ccc(-c2cccc3ccccc23)c(-c2cc(-c3cccc4c3sc3ccccc34)nc(-c3cccc4sc5ccccc5c34)n2)c1 ; c1ccc(-c2ccccc2-c2cc(-c3cccc4c3sc3ccccc34)nc(-c3ccc4sc5ccccc5c4c3)n2)cc1 ; c1ccc(-c2ccccc2-c2cc(-c3cccc4c3sc3ccccc34)nc(-c3ccc4c5ccccc5c5ccccc5c4c3)n2)cc1]

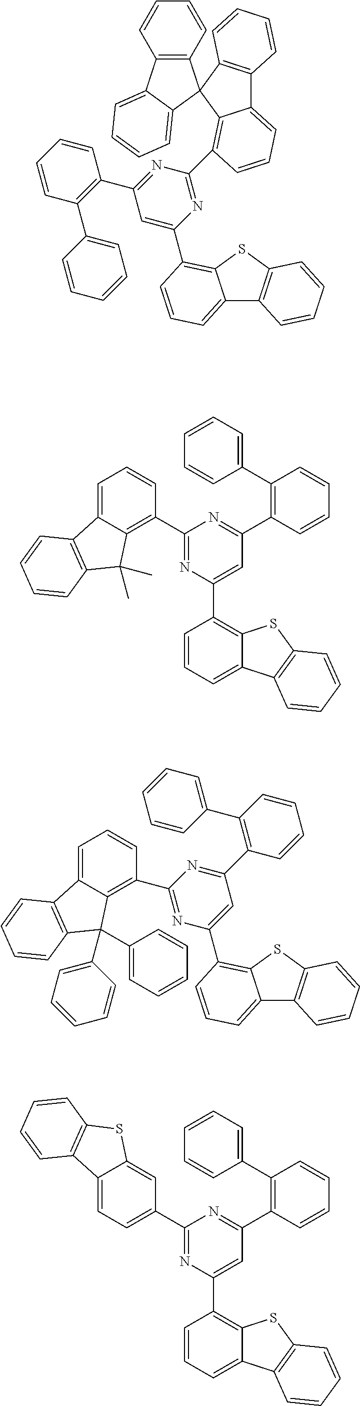 [SMILES: c1ccc(-c2ccccc2-c2cc(-c3cccc4c3sc3ccccc34)nc(-c3cccc4c3C3(c5ccccc5-c5ccccc53)c3ccccc3-4)n2)cc1 ; CC1(C)c2ccccc2-c2cccc(-c3nc(-c4ccccc4-c4ccccc4)cc(-c4cccc5c4sc4ccccc45)n3)c21 ; c1ccc(-c2ccccc2-c2cc(-c3cccc4c3sc3ccccc34)nc(-c3cccc4c3C(c3ccccc3)(c3ccccc3)c3ccccc3-4)n2)cc1 ; c1ccc(-c2ccccc2-c2cc(-c3cccc4c3sc3ccccc34)nc(-c3ccc4c(c3)sc3ccccc34)n2)cc1]

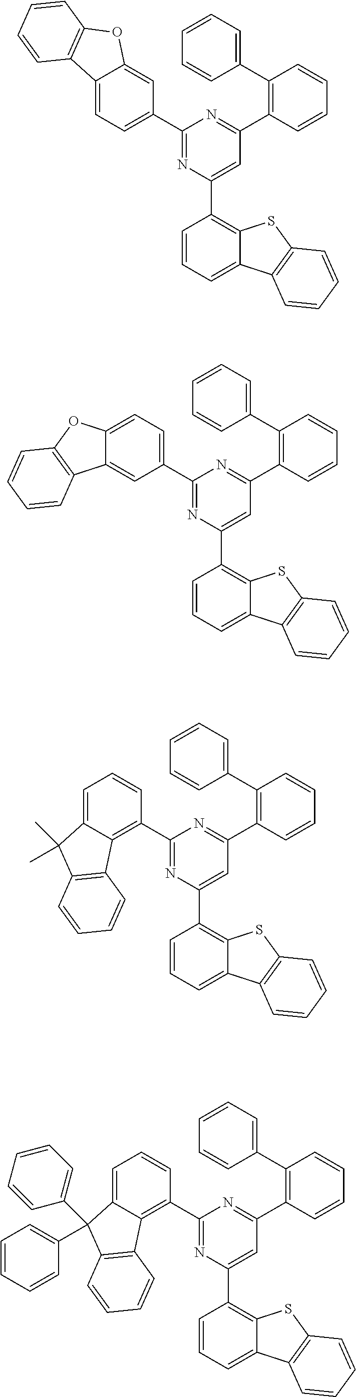 [SMILES: c1ccc(-c2ccccc2-c2cc(-c3cccc4c3sc3ccccc34)nc(-c3ccc4c(c3)oc3ccccc34)n2)cc1 ; c1ccc(-c2ccccc2-c2cc(-c3cccc4c3sc3ccccc34)nc(-c3ccc4oc5ccccc5c4c3)n2)cc1 ; CC1(C)c2ccccc2-c2c(-c3nc(-c4ccccc4-c4ccccc4)cc(-c4cccc5c4sc4ccccc45)n3)cccc21 ; c1ccc(-c2ccccc2-c2cc(-c3cccc4c3sc3ccccc34)nc(-c3cccc4c3-c3ccccc3C4(c3ccccc3)c3ccccc3)n2)cc1]

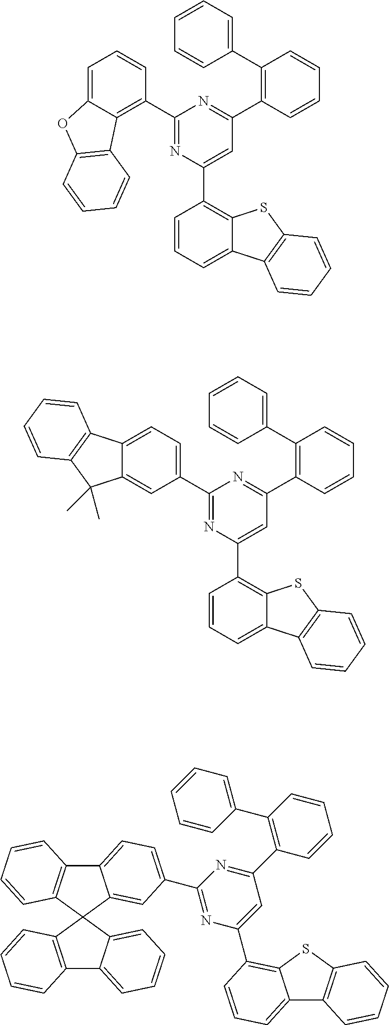 [SMILES: c1ccc(-c2ccccc2-c2cc(-c3cccc4c3sc3ccccc34)nc(-c3cccc4oc5ccccc5c34)n2)cc1 ; CC1(C)c2ccccc2-c2ccc(-c3nc(-c4ccccc4-c4ccccc4)cc(-c4cccc5c4sc4ccccc45)n3)cc21 ; c1ccc(-c2ccccc2-c2cc(-c3cccc4c3sc3ccccc34)nc(-c3ccc4c(c3)C3(c5ccccc5-c5ccccc53)c3ccccc3-4)n2)cc1]

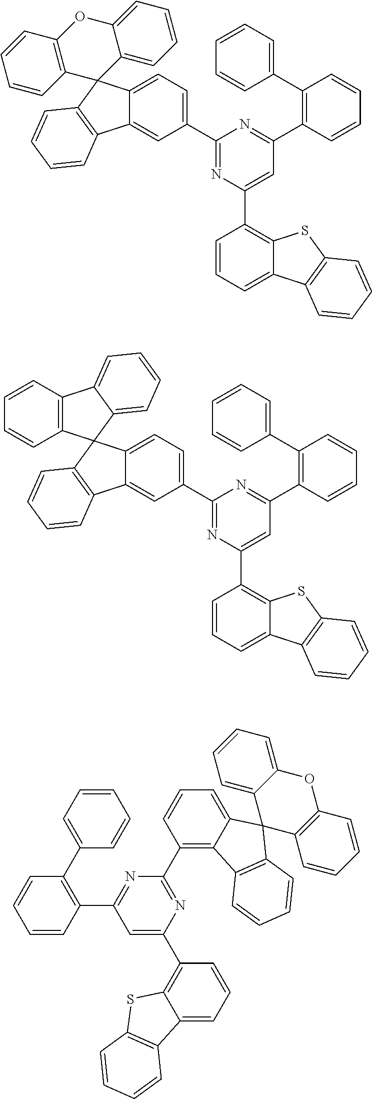 [SMILES: c1ccc(-c2ccccc2-c2cc(-c3cccc4c3sc3ccccc34)nc(-c3ccc4c(c3)-c3ccccc3C43c4ccccc4Oc4ccccc43)n2)cc1 ; c1ccc(-c2ccccc2-c2cc(-c3cccc4c3sc3ccccc34)nc(-c3ccc4c(c3)-c3ccccc3C43c4ccccc4-c4ccccc43)n2)cc1 ; c1ccc(-c2ccccc2-c2cc(-c3cccc4c3sc3ccccc34)nc(-c3cccc4c3-c3ccccc3C43c4ccccc4Oc4ccccc43)n2)cc1]

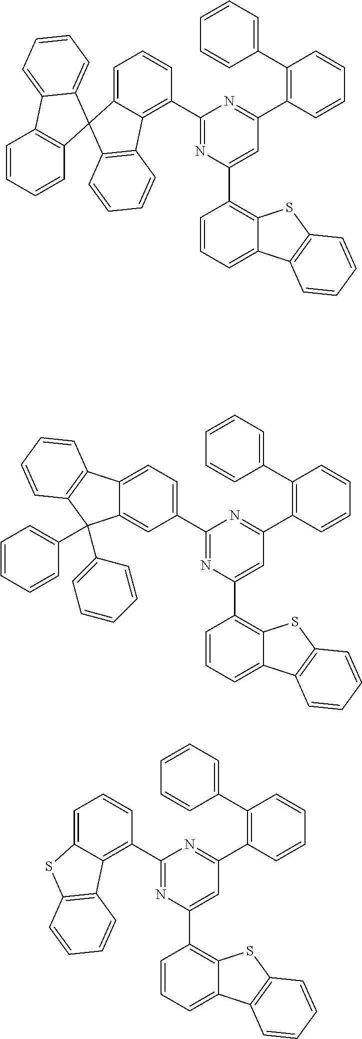 [SMILES: c1ccc(-c2ccccc2-c2cc(-c3cccc4c3sc3ccccc34)nc(-c3cccc4c3-c3ccccc3C43c4ccccc4-c4ccccc43)n2)cc1 ; c1ccc(-c2ccccc2-c2cc(-c3cccc4c3sc3ccccc34)nc(-c3ccc4c(c3)C(c3ccccc3)(c3ccccc3)c3ccccc3-4)n2)cc1 ; c1ccc(-c2ccccc2-c2cc(-c3cccc4c3sc3ccccc34)nc(-c3cccc4sc5ccccc5c34)n2)cc1]

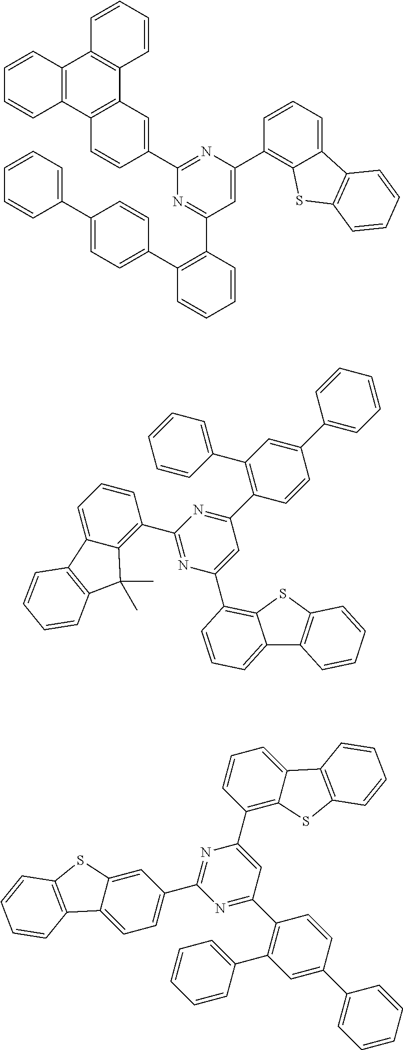 [SMILES: c1ccc(-c2ccc(-c3ccccc3-c3cc(-c4cccc5c4sc4ccccc45)nc(-c4ccc5c6ccccc6c6ccccc6c5c4)n3)cc2)cc1 ; CC1(C)c2ccccc2-c2cccc(-c3nc(-c4ccc(-c5ccccc5)cc4-c4ccccc4)cc(-c4cccc5c4sc4ccccc45)n3)c21 ; c1ccc(-c2ccc(-c3cc(-c4cccc5c4sc4ccccc45)nc(-c4ccc5c(c4)sc4ccccc45)n3)c(-c3ccccc3)c2)cc1]

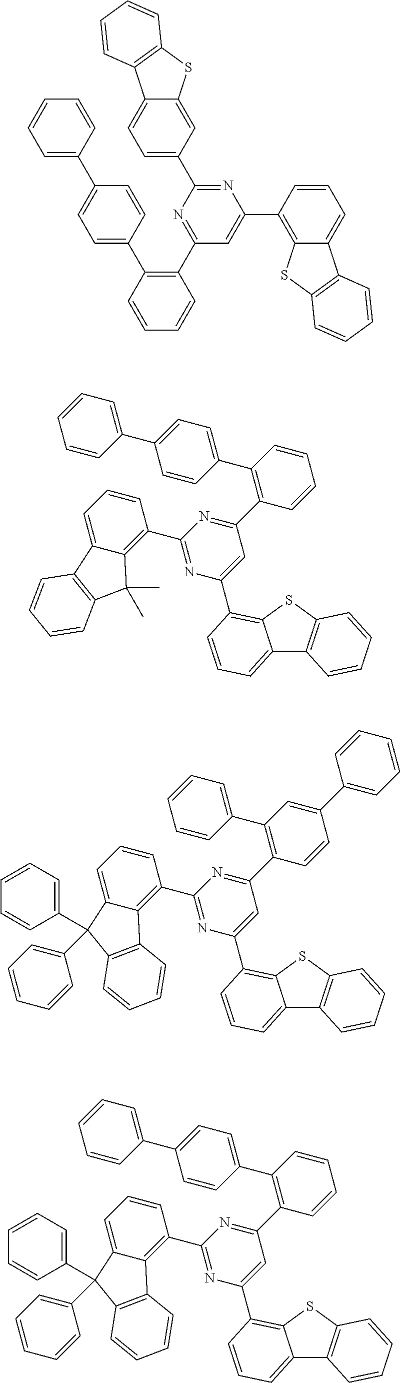 [SMILES: c1ccc(-c2ccc(-c3ccccc3-c3cc(-c4cccc5c4sc4ccccc45)nc(-c4ccc5c(c4)sc4ccccc45)n3)cc2)cc1 ; CC1(C)c2ccccc2-c2cccc(-c3nc(-c4ccccc4-c4ccc(-c5ccccc5)cc4)cc(-c4cccc5c4sc4ccccc45)n3)c21 ; c1ccc(-c2ccc(-c3cc(-c4cccc5c4sc4ccccc45)nc(-c4cccc5c4-c4ccccc4C5(c4ccccc4)c4ccccc4)n3)c(-c3ccccc3)c2)cc1 ; c1ccc(-c2ccc(-c3ccccc3-c3cc(-c4cccc5c4sc4ccccc45)nc(-c4cccc5c4-c4ccccc4C5(c4ccccc4)c4ccccc4)n3)cc2)cc1]

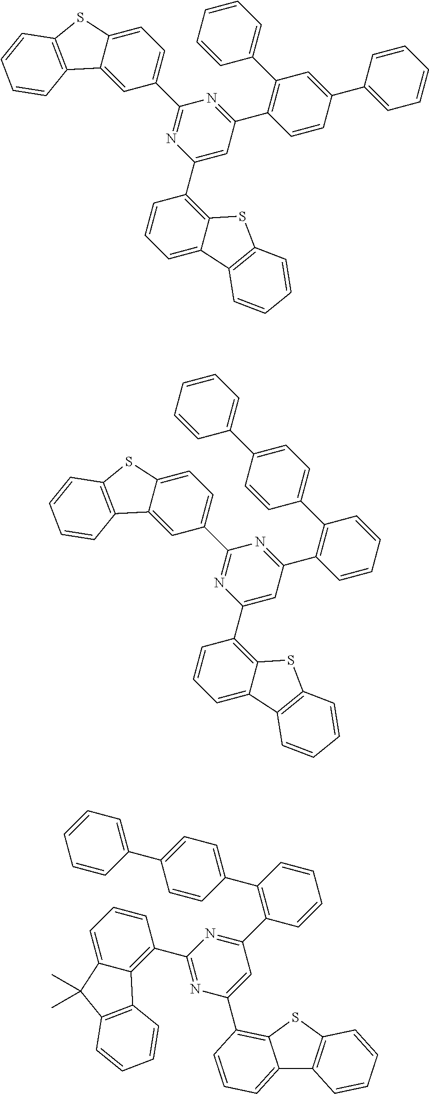 [SMILES: c1ccc(-c2ccc(-c3cc(-c4cccc5c4sc4ccccc45)nc(-c4ccc5sc6ccccc6c5c4)n3)c(-c3ccccc3)c2)cc1 ; c1ccc(-c2ccc(-c3ccccc3-c3cc(-c4cccc5c4sc4ccccc45)nc(-c4ccc5sc6ccccc6c5c4)n3)cc2)cc1 ; CC1(C)c2ccccc2-c2c(-c3nc(-c4ccccc4-c4ccc(-c5ccccc5)cc4)cc(-c4cccc5c4sc4ccccc45)n3)cccc21]

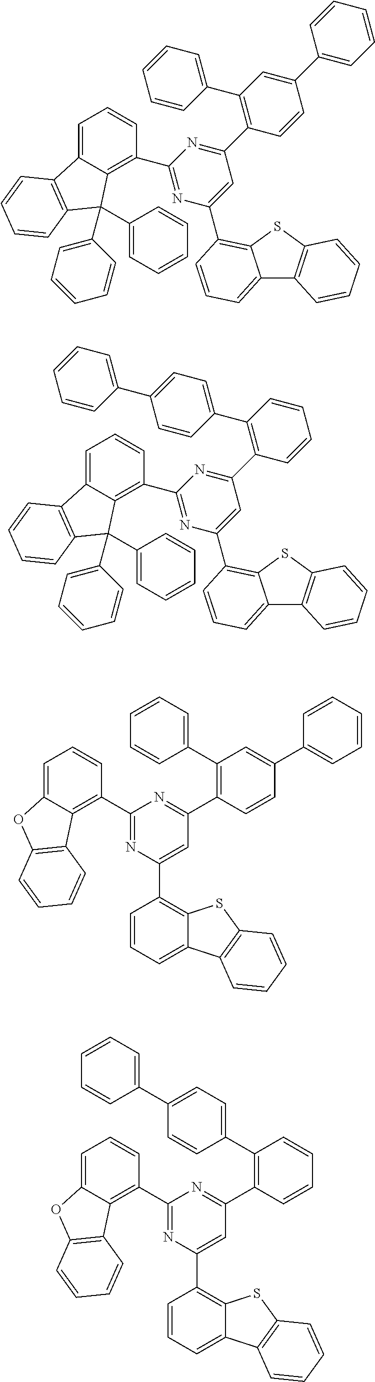

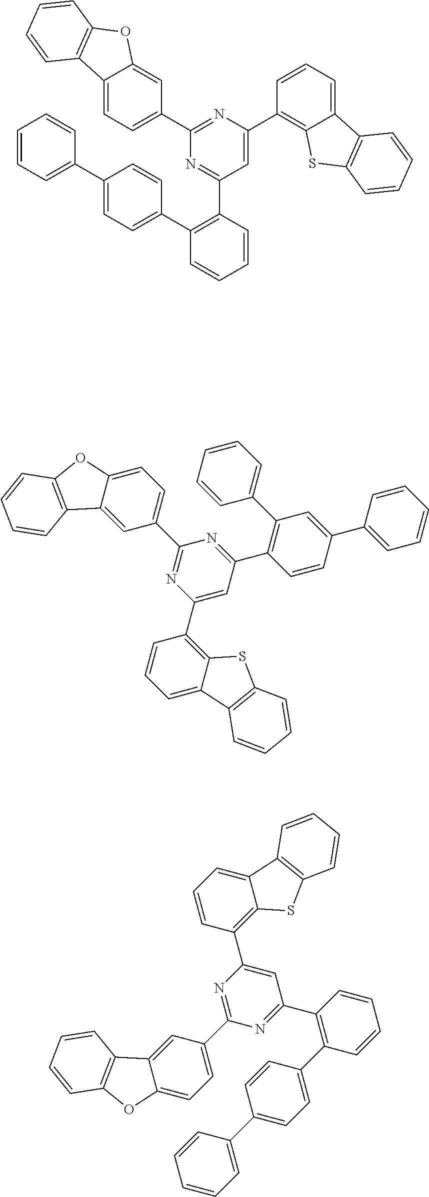 [SMILES: c1ccc(-c2ccc(-c3ccccc3-c3cc(-c4cccc5c4sc4ccccc45)nc(-c4ccc5c(c4)oc4ccccc45)n3)cc2)cc1 ; c1ccc(-c2ccc(-c3cc(-c4cccc5c4sc4ccccc45)nc(-c4ccc5oc6ccccc6c5c4)n3)c(-c3ccccc3)c2)cc1 ; c1ccc(-c2ccc(-c3ccccc3-c3cc(-c4cccc5c4sc4ccccc45)nc(-c4ccc5oc6ccccc6c5c4)n3)cc2)cc1]

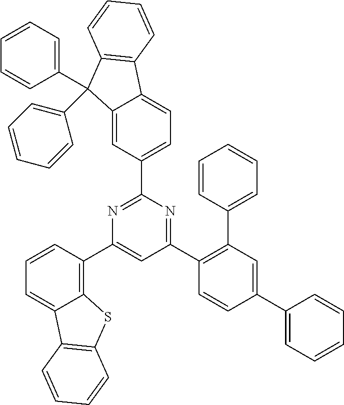 [SMILES: c1ccc(-c2ccc(-c3cc(-c4cccc5c4sc4ccccc45)nc(-c4ccc5c(c4)C(c4ccccc4)(c4ccccc4)c4ccccc4-5)n3)c(-c3ccccc3)c2)cc1]

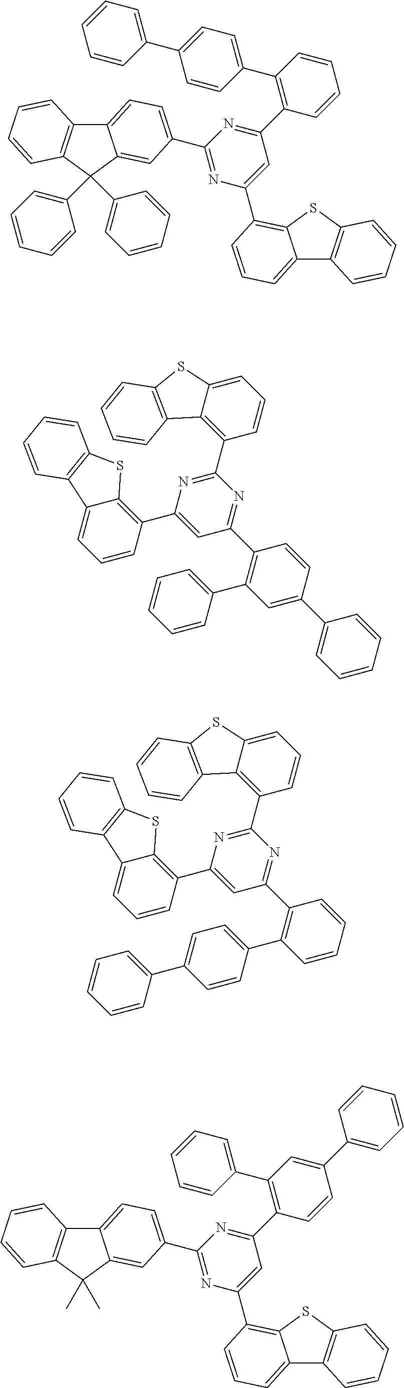 [SMILES: c1ccc(-c2ccc(-c3ccccc3-c3cc(-c4cccc5c4sc4ccccc45)nc(-c4ccc5c(c4)C(c4ccccc4)(c4ccccc4)c4ccccc4-5)n3)cc2)cc1 ; c1ccc(-c2ccc(-c3cc(-c4cccc5c4sc4ccccc45)nc(-c4cccc5sc6ccccc6c45)n3)c(-c3ccccc3)c2)cc1 ; c1ccc(-c2ccc(-c3ccccc3-c3cc(-c4cccc5c4sc4ccccc45)nc(-c4cccc5sc6ccccc6c45)n3)cc2)cc1 ; CC1(C)c2ccccc2-c2ccc(-c3nc(-c4ccc(-c5ccccc5)cc4-c4ccccc4)cc(-c4cccc5c4sc4ccccc45)n3)cc21]

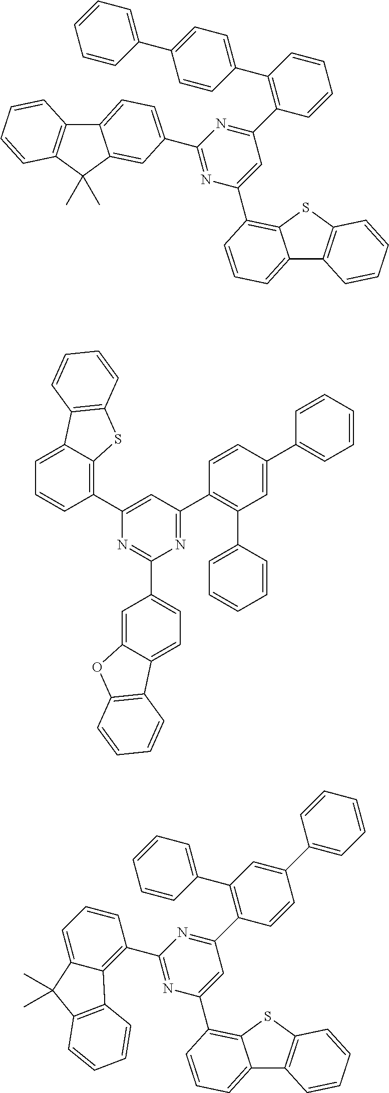 [SMILES: CC1(C)c2ccccc2-c2ccc(-c3nc(-c4ccccc4-c4ccc(-c5ccccc5)cc4)cc(-c4cccc5c4sc4ccccc45)n3)cc21 ; c1ccc(-c2ccc(-c3cc(-c4cccc5c4sc4ccccc45)nc(-c4ccc5c(c4)oc4ccccc45)n3)c(-c3ccccc3)c2)cc1 ; CC1(C)c2ccccc2-c2c(-c3nc(-c4ccc(-c5ccccc5)cc4-c4ccccc4)cc(-c4cccc5c4sc4ccccc45)n3)cccc21]

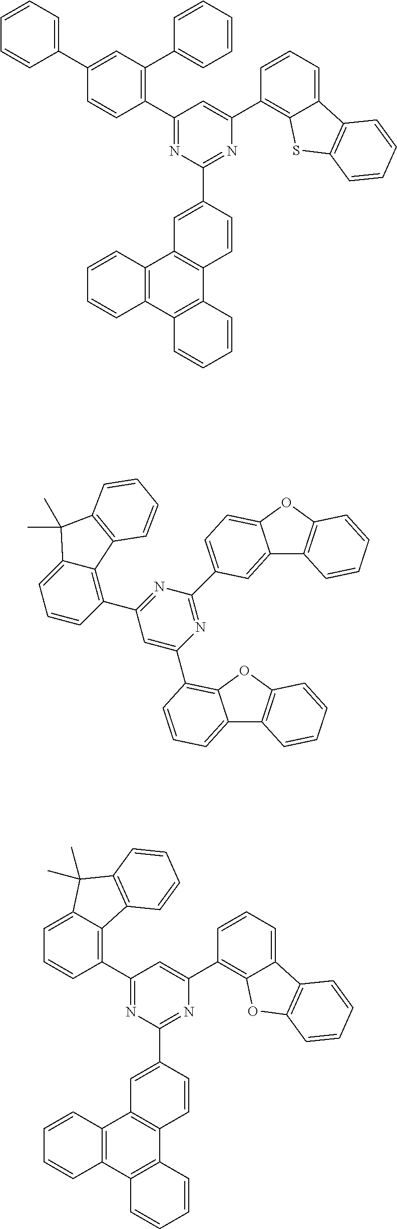 [SMILES: c1ccc(-c2ccc(-c3cc(-c4cccc5c4sc4ccccc45)nc(-c4ccc5c6ccccc6c6ccccc6c5c4)n3)c(-c3ccccc3)c2)cc1 ; CC1(C)c2ccccc2-c2c(-c3cc(-c4cccc5c4oc4ccccc45)nc(-c4ccc5oc6ccccc6c5c4)n3)cccc21 ; CC1(C)c2ccccc2-c2c(-c3cc(-c4cccc5c4oc4ccccc45)nc(-c4ccc5c6ccccc6c6ccccc6c5c4)n3)cccc21]

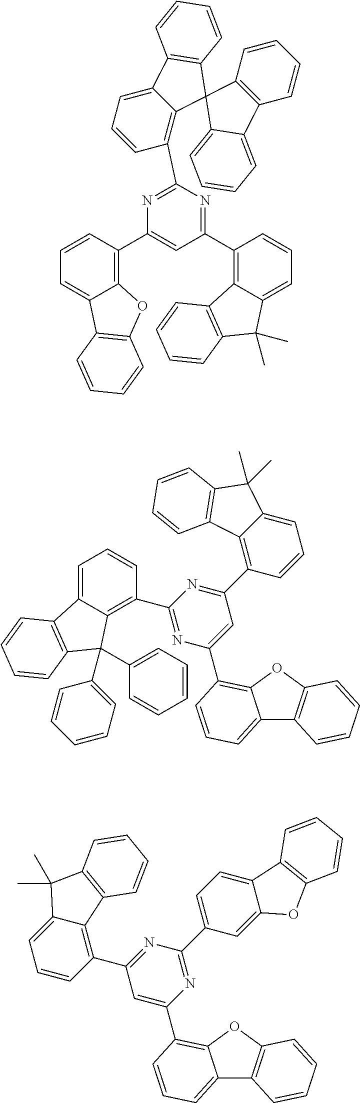 [SMILES: CC1(C)c2ccccc2-c2c(-c3cc(-c4cccc5c4oc4ccccc45)nc(-c4cccc5c4C4(c6ccccc6-c6ccccc64)c4ccccc4-5)n3)cccc21 ; CC1(C)c2ccccc2-c2c(-c3cc(-c4cccc5c4oc4ccccc45)nc(-c4cccc5c4C(c4ccccc4)(c4ccccc4)c4ccccc4-5)n3)cccc21 ; CC1(C)c2ccccc2-c2c(-c3cc(-c4cccc5c4oc4ccccc45)nc(-c4ccc5c(c4)oc4ccccc45)n3)cccc21]

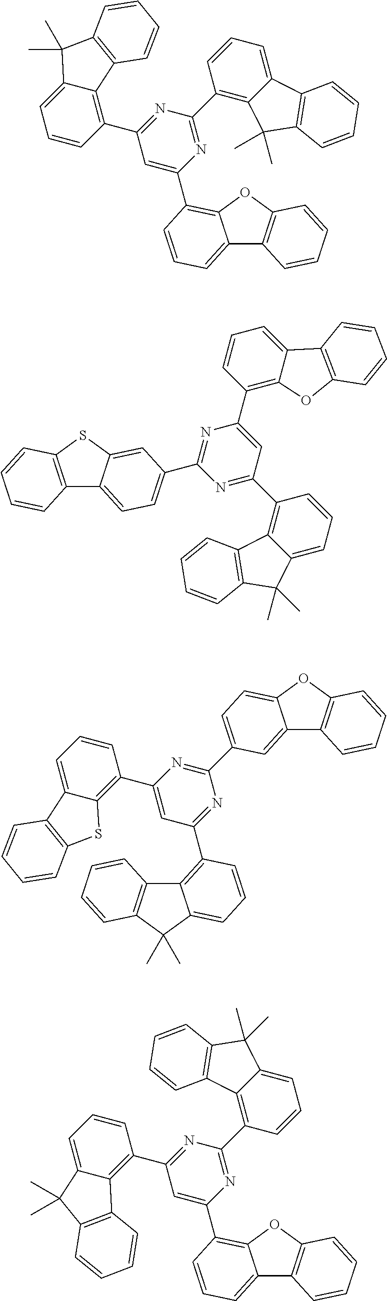 [SMILES: CC1(C)c2ccccc2-c2c(-c3cc(-c4cccc5c4oc4ccccc45)nc(-c4cccc5c4C(C)(C)c4ccccc4-5)n3)cccc21 ; CC1(C)c2ccccc2-c2c(-c3cc(-c4cccc5c4oc4ccccc45)nc(-c4ccc5c(c4)sc4ccccc45)n3)cccc21 ; CC1(C)c2ccccc2-c2c(-c3cc(-c4cccc5c4sc4ccccc45)nc(-c4ccc5oc6ccccc6c5c4)n3)cccc21 ; CC1(C)c2ccccc2-c2c(-c3cc(-c4cccc5c4oc4ccccc45)nc(-c4cccc5c4-c4ccccc4C5(C)C)n3)cccc21]

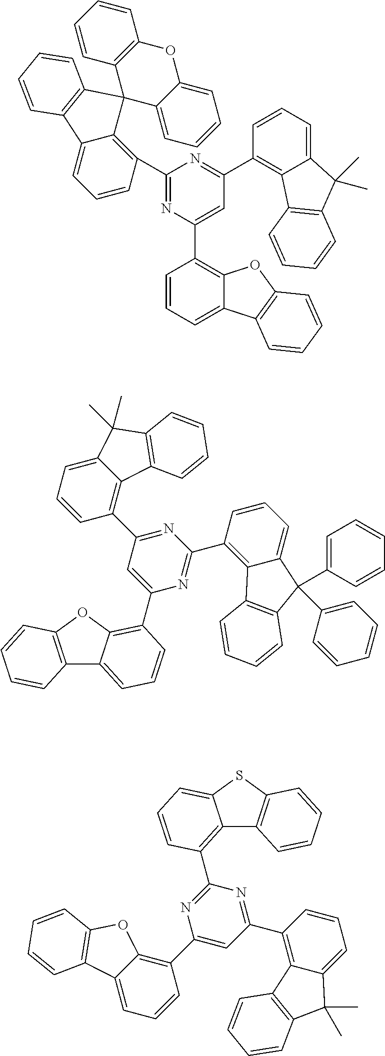 [SMILES: CC1(C)c2ccccc2-c2c(-c3cc(-c4cccc5c4oc4ccccc45)nc(-c4cccc5c4C4(c6ccccc6Oc6ccccc64)c4ccccc4-5)n3)cccc21 ; CC1(C)c2ccccc2-c2c(-c3cc(-c4cccc5c4oc4ccccc45)nc(-c4cccc5c4-c4ccccc4C5(c4ccccc4)c4ccccc4)n3)cccc21 ; CC1(C)c2ccccc2-c2c(-c3cc(-c4cccc5c4oc4ccccc45)nc(-c4cccc5sc6ccccc6c45)n3)cccc21]

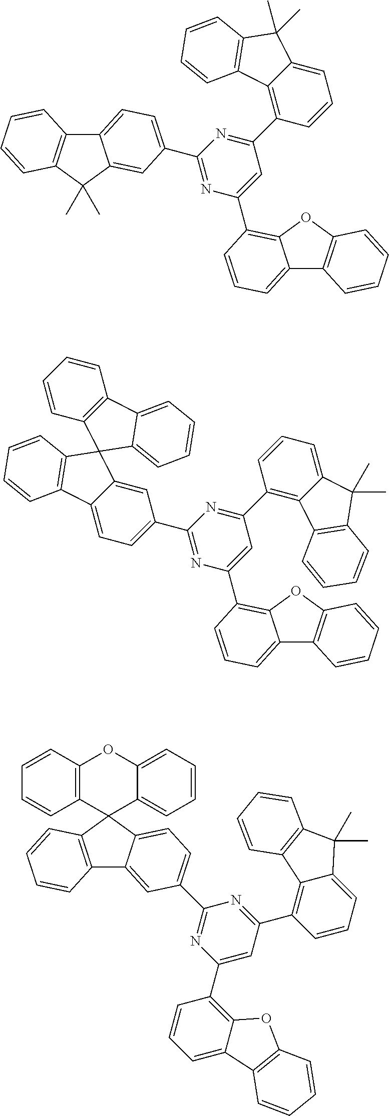 [SMILES: CC1(C)c2ccccc2-c2ccc(-c3nc(-c4cccc5c4-c4ccccc4C5(C)C)cc(-c4cccc5c4oc4ccccc45)n3)cc21 ; CC1(C)c2ccccc2-c2c(-c3cc(-c4cccc5c4oc4ccccc45)nc(-c4ccc5c(c4)C4(c6ccccc6-c6ccccc64)c4ccccc4-5)n3)cccc21 ; CC1(C)c2ccccc2-c2c(-c3cc(-c4cccc5c4oc4ccccc45)nc(-c4ccc5c(c4)-c4ccccc4C54c5ccccc5Oc5ccccc54)n3)cccc21]

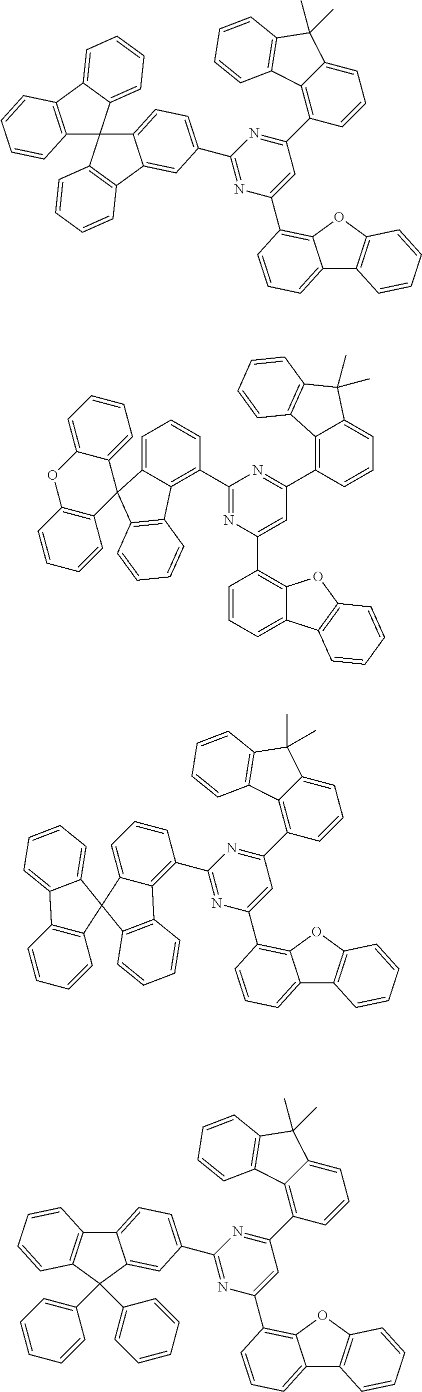 [SMILES: CC1(C)c2ccccc2-c2c(-c3cc(-c4cccc5c4oc4ccccc45)nc(-c4ccc5c(c4)-c4ccccc4C54c5ccccc5-c5ccccc54)n3)cccc21 ; CC1(C)c2ccccc2-c2c(-c3cc(-c4cccc5c4oc4ccccc45)nc(-c4cccc5c4-c4ccccc4C54c5ccccc5Oc5ccccc54)n3)cccc21 ; CC1(C)c2ccccc2-c2c(-c3cc(-c4cccc5c4oc4ccccc45)nc(-c4cccc5c4-c4ccccc4C54c5ccccc5-c5ccccc54)n3)cccc21 ; CC1(C)c2ccccc2-c2c(-c3cc(-c4cccc5c4oc4ccccc45)nc(-c4ccc5c(c4)C(c4ccccc4)(c4ccccc4)c4ccccc4-5)n3)cccc21]

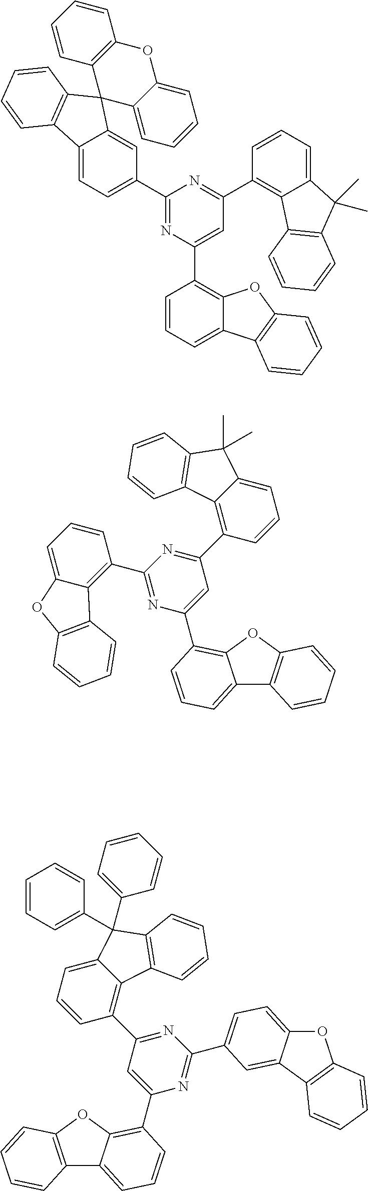 [SMILES: CC1(C)c2ccccc2-c2c(-c3cc(-c4cccc5c4oc4ccccc45)nc(-c4ccc5c(c4)C4(c6ccccc6Oc6ccccc64)c4ccccc4-5)n3)cccc21 ; CC1(C)c2ccccc2-c2c(-c3cc(-c4cccc5c4oc4ccccc45)nc(-c4cccc5oc6ccccc6c45)n3)cccc21 ; c1ccc(C2(c3ccccc3)c3ccccc3-c3c(-c4cc(-c5cccc6c5oc5ccccc56)nc(-c5ccc6oc7ccccc7c6c5)n4)cccc32)cc1]

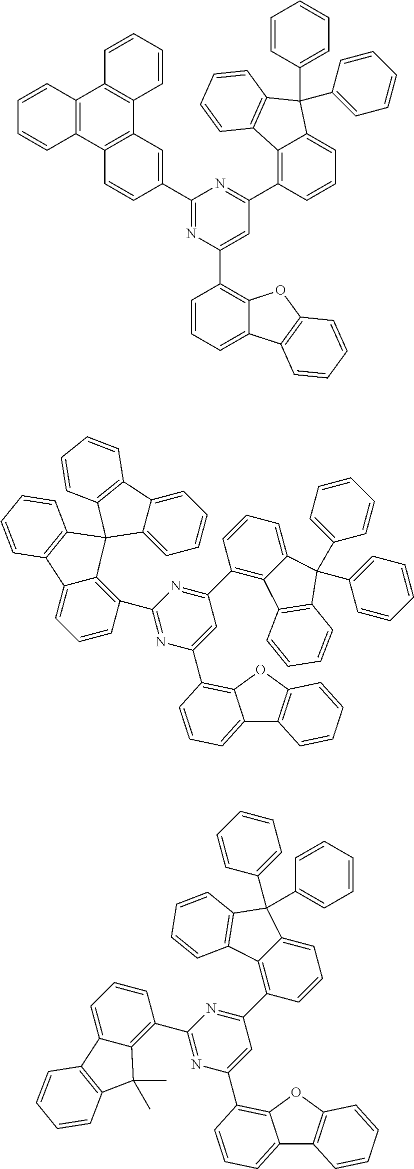 [SMILES: c1ccc(C2(c3ccccc3)c3ccccc3-c3c(-c4cc(-c5cccc6c5oc5ccccc56)nc(-c5ccc6c7ccccc7c7ccccc7c6c5)n4)cccc32)cc1 ; c1ccc(C2(c3ccccc3)c3ccccc3-c3c(-c4cc(-c5cccc6c5oc5ccccc56)nc(-c5cccc6c5C5(c7ccccc7-c7ccccc75)c5ccccc5-6)n4)cccc32)cc1 ; CC1(C)c2ccccc2-c2cccc(-c3nc(-c4cccc5c4-c4ccccc4C5(c4ccccc4)c4ccccc4)cc(-c4cccc5c4oc4ccccc45)n3)c21]

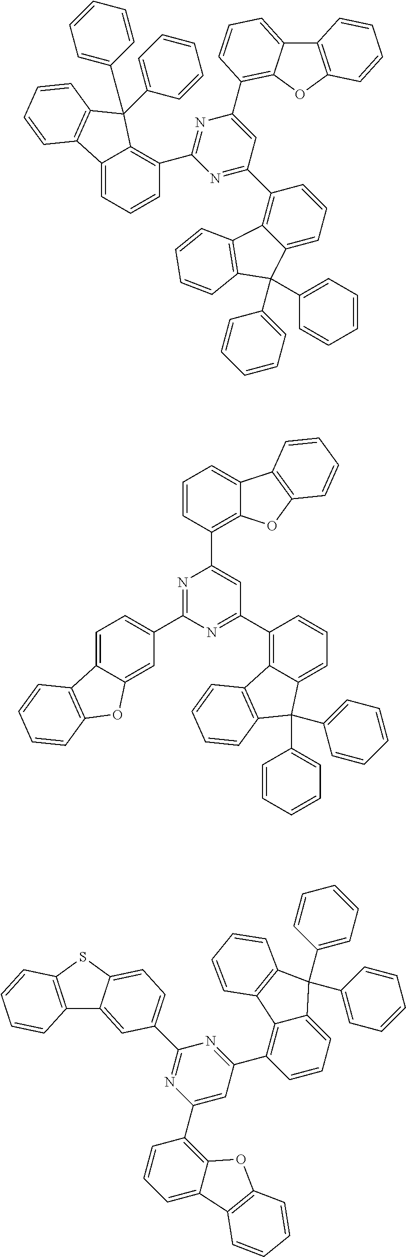 [SMILES: c1ccc(C2(c3ccccc3)c3ccccc3-c3c(-c4cc(-c5cccc6c5oc5ccccc56)nc(-c5cccc6c5C(c5ccccc5)(c5ccccc5)c5ccccc5-6)n4)cccc32)cc1 ; c1ccc(C2(c3ccccc3)c3ccccc3-c3c(-c4cc(-c5cccc6c5oc5ccccc56)nc(-c5ccc6c(c5)oc5ccccc56)n4)cccc32)cc1 ; c1ccc(C2(c3ccccc3)c3ccccc3-c3c(-c4cc(-c5cccc6c5oc5ccccc56)nc(-c5ccc6sc7ccccc7c6c5)n4)cccc32)cc1]

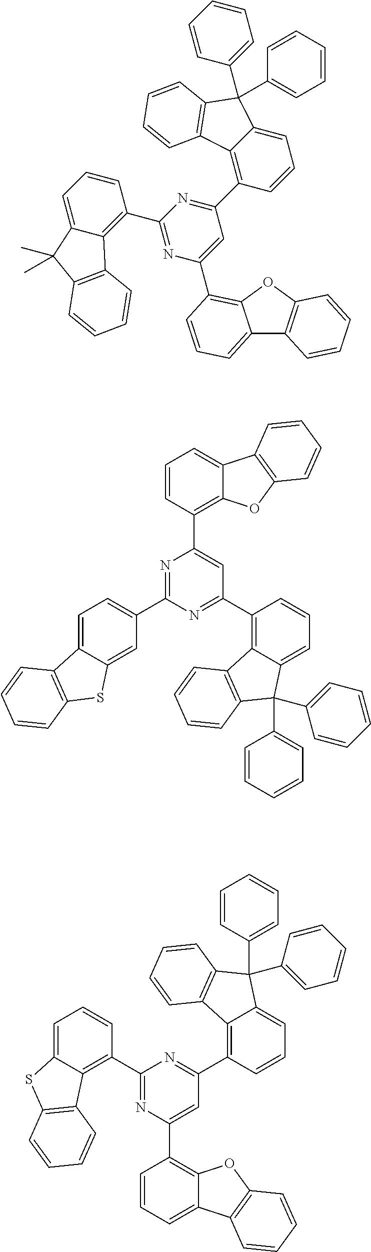 [SMILES: CC1(C)c2ccccc2-c2c(-c3nc(-c4cccc5c4-c4ccccc4C5(c4ccccc4)c4ccccc4)cc(-c4cccc5c4oc4ccccc45)n3)cccc21 ; c1ccc(C2(c3ccccc3)c3ccccc3-c3c(-c4cc(-c5cccc6c5oc5ccccc56)nc(-c5ccc6c(c5)sc5ccccc56)n4)cccc32)cc1 ; c1ccc(C2(c3ccccc3)c3ccccc3-c3c(-c4cc(-c5cccc6c5oc5ccccc56)nc(-c5cccc6sc7ccccc7c56)n4)cccc32)cc1]

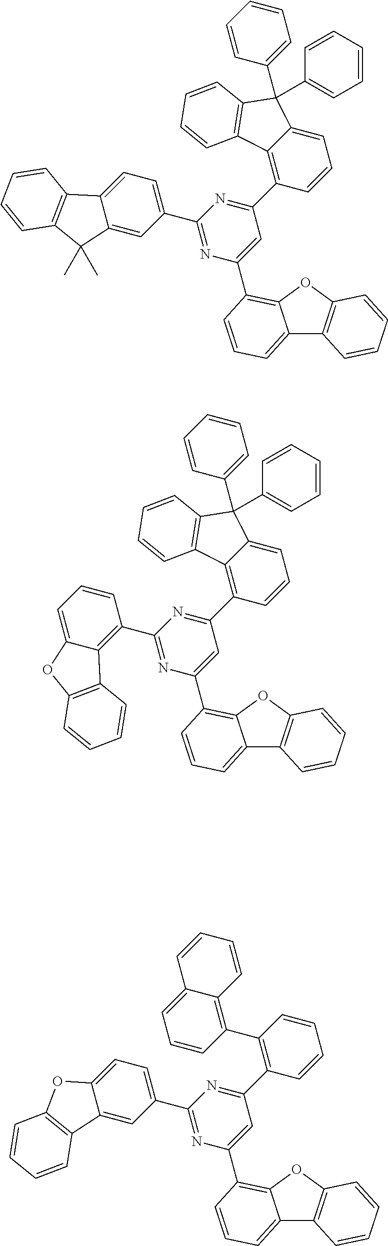 [SMILES: CC1(C)c2ccccc2-c2ccc(-c3nc(-c4cccc5c4-c4ccccc4C5(c4ccccc4)c4ccccc4)cc(-c4cccc5c4oc4ccccc45)n3)cc21 ; c1ccc(C2(c3ccccc3)c3ccccc3-c3c(-c4cc(-c5cccc6c5oc5ccccc56)nc(-c5cccc6oc7ccccc7c56)n4)cccc32)cc1 ; c1ccc(-c2cccc3ccccc23)c(-c2cc(-c3cccc4c3oc3ccccc34)nc(-c3ccc4oc5ccccc5c4c3)n2)c1]

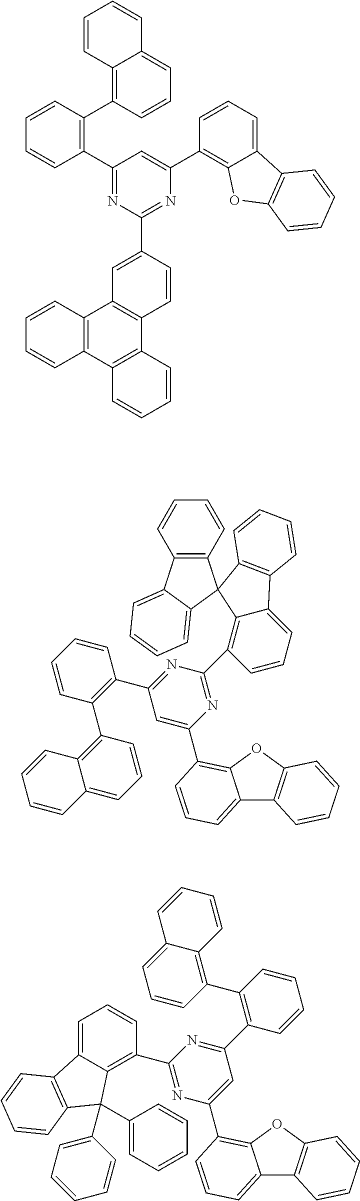 [SMILES: c1ccc(-c2cccc3ccccc23)c(-c2cc(-c3cccc4c3oc3ccccc34)nc(-c3ccc4c5ccccc5c5ccccc5c4c3)n2)c1 ; c1ccc(-c2cccc3ccccc23)c(-c2cc(-c3cccc4c3oc3ccccc34)nc(-c3cccc4c3C3(c5ccccc5-c5ccccc53)c3ccccc3-4)n2)c1 ; c1ccc(C2(c3ccccc3)c3ccccc3-c3cccc(-c4nc(-c5ccccc5-c5cccc6ccccc56)cc(-c5cccc6c5oc5ccccc56)n4)c32)cc1]

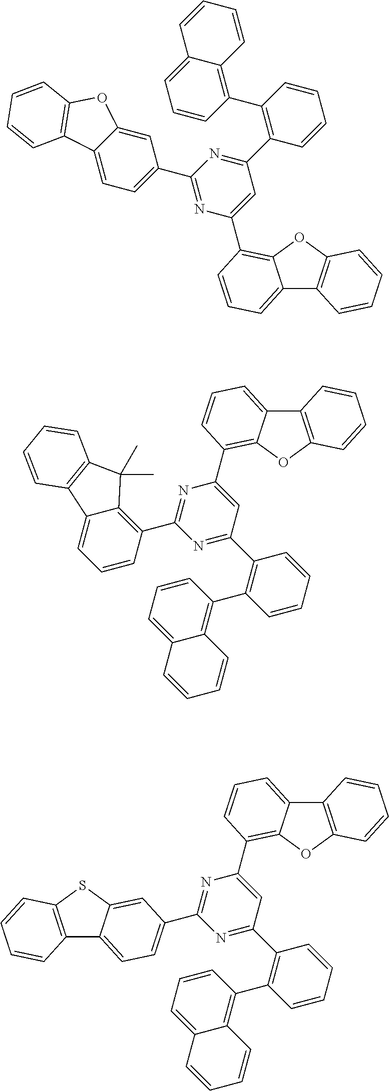 [SMILES: c1ccc(-c2cccc3ccccc23)c(-c2cc(-c3cccc4c3oc3ccccc34)nc(-c3ccc4c(c3)oc3ccccc34)n2)c1 ; CC1(C)c2ccccc2-c2cccc(-c3nc(-c4ccccc4-c4cccc5ccccc45)cc(-c4cccc5c4oc4ccccc45)n3)c21 ; c1ccc(-c2cccc3ccccc23)c(-c2cc(-c3cccc4c3oc3ccccc34)nc(-c3ccc4c(c3)sc3ccccc34)n2)c1]

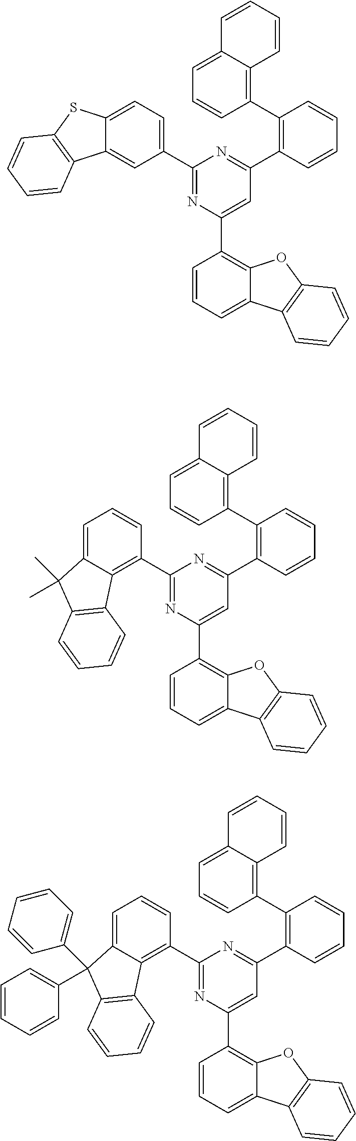 [SMILES: c1ccc(-c2cccc3ccccc23)c(-c2cc(-c3cccc4c3oc3ccccc34)nc(-c3ccc4sc5ccccc5c4c3)n2)c1 ; CC1(C)c2ccccc2-c2c(-c3nc(-c4ccccc4-c4cccc5ccccc45)cc(-c4cccc5c4oc4ccccc45)n3)cccc21 ; c1ccc(C2(c3ccccc3)c3ccccc3-c3c(-c4nc(-c5ccccc5-c5cccc6ccccc56)cc(-c5cccc6c5oc5ccccc56)n4)cccc32)cc1]

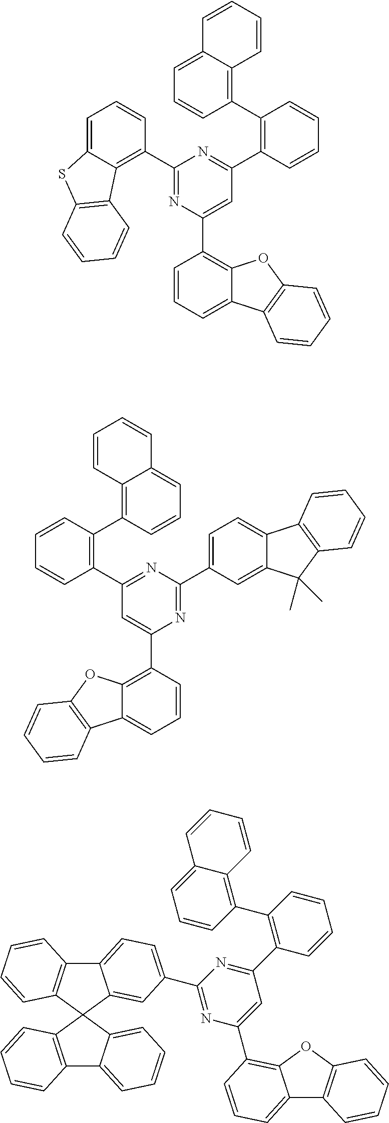 [SMILES: c1ccc(-c2cccc3ccccc23)c(-c2cc(-c3cccc4c3oc3ccccc34)nc(-c3cccc4sc5ccccc5c34)n2)c1 ; CC1(C)c2ccccc2-c2ccc(-c3nc(-c4ccccc4-c4cccc5ccccc45)cc(-c4cccc5c4oc4ccccc45)n3)cc21 ; c1ccc(-c2cccc3ccccc23)c(-c2cc(-c3cccc4c3oc3ccccc34)nc(-c3ccc4c(c3)C3(c5ccccc5-c5ccccc53)c3ccccc3-4)n2)c1]

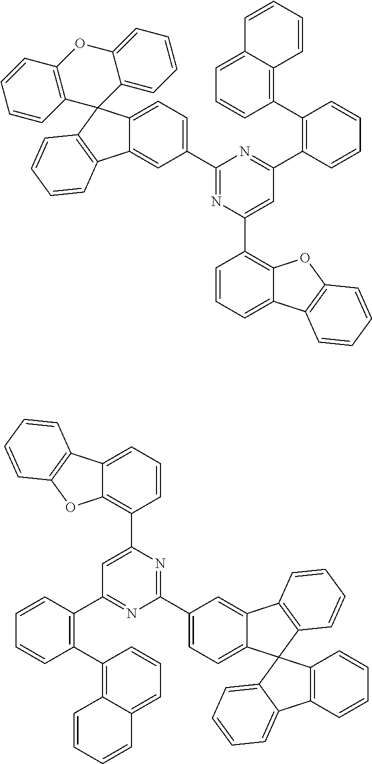 [SMILES: c1ccc2c(c1)Oc1ccccc1C21c2ccccc2-c2cc(-c3nc(-c4ccccc4-c4cccc5ccccc45)cc(-c4cccc5c4oc4ccccc45)n3)ccc21 ; c1ccc(-c2cccc3ccccc23)c(-c2cc(-c3cccc4c3oc3ccccc34)nc(-c3ccc4c(c3)-c3ccccc3C43c4ccccc4-c4ccccc43)n2)c1]

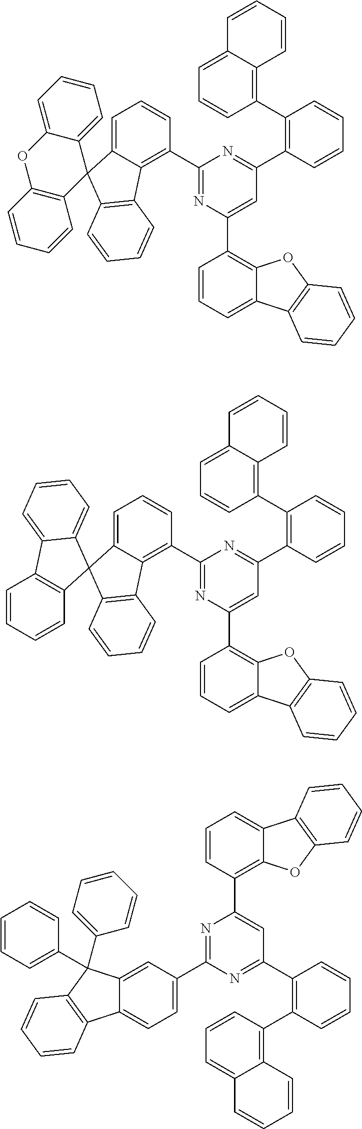

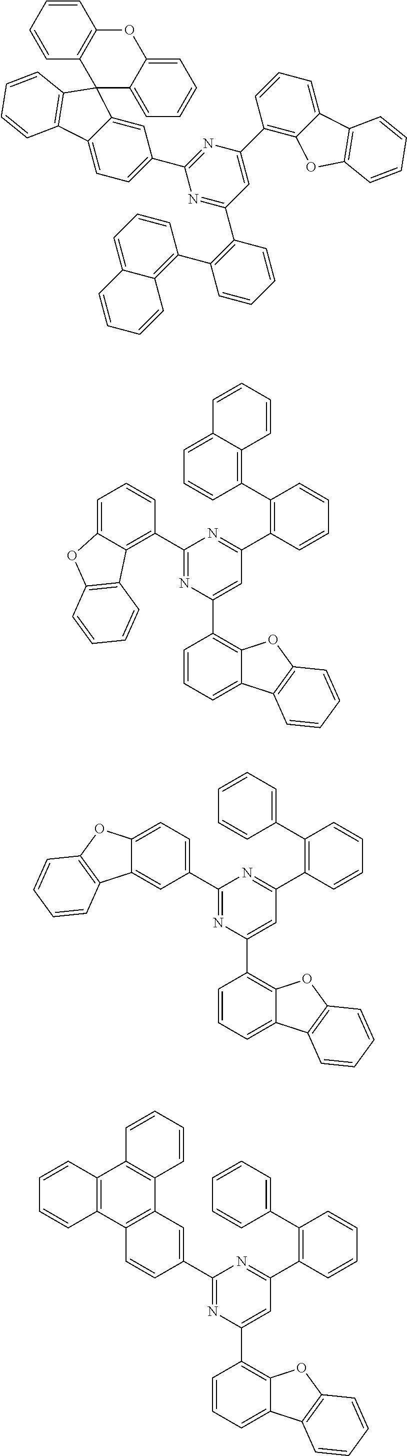 [SMILES: c1ccc2c(c1)Oc1ccccc1C21c2ccccc2-c2ccc(-c3nc(-c4ccccc4-c4cccc5ccccc45)cc(-c4cccc5c4oc4ccccc45)n3)cc21 ; c1ccc(-c2cccc3ccccc23)c(-c2cc(-c3cccc4c3oc3ccccc34)nc(-c3cccc4oc5ccccc5c34)n2)c1 ; c1ccc(-c2ccccc2-c2cc(-c3cccc4c3oc3ccccc34)nc(-c3ccc4oc5ccccc5c4c3)n2)cc1 ; c1ccc(-c2ccccc2-c2cc(-c3cccc4c3oc3ccccc34)nc(-c3ccc4c5ccccc5c5ccccc5c4c3)n2)cc1]

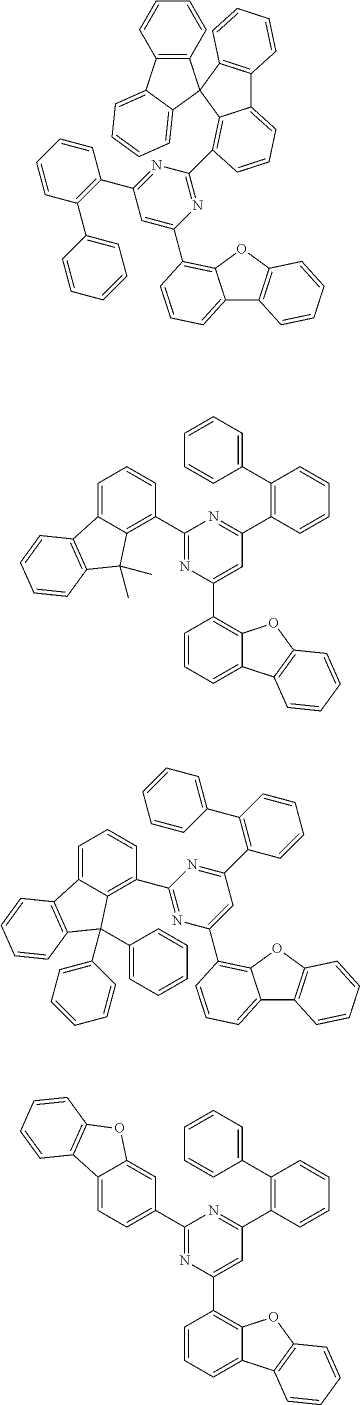 [SMILES: c1ccc(-c2ccccc2-c2cc(-c3cccc4c3oc3ccccc34)nc(-c3cccc4c3C3(c5ccccc5-c5ccccc53)c3ccccc3-4)n2)cc1 ; CC1(C)c2ccccc2-c2cccc(-c3nc(-c4ccccc4-c4ccccc4)cc(-c4cccc5c4oc4ccccc45)n3)c21 ; c1ccc(-c2ccccc2-c2cc(-c3cccc4c3oc3ccccc34)nc(-c3cccc4c3C(c3ccccc3)(c3ccccc3)c3ccccc3-4)n2)cc1 ; c1ccc(-c2ccccc2-c2cc(-c3cccc4c3oc3ccccc34)nc(-c3ccc4c(c3)oc3ccccc34)n2)cc1]

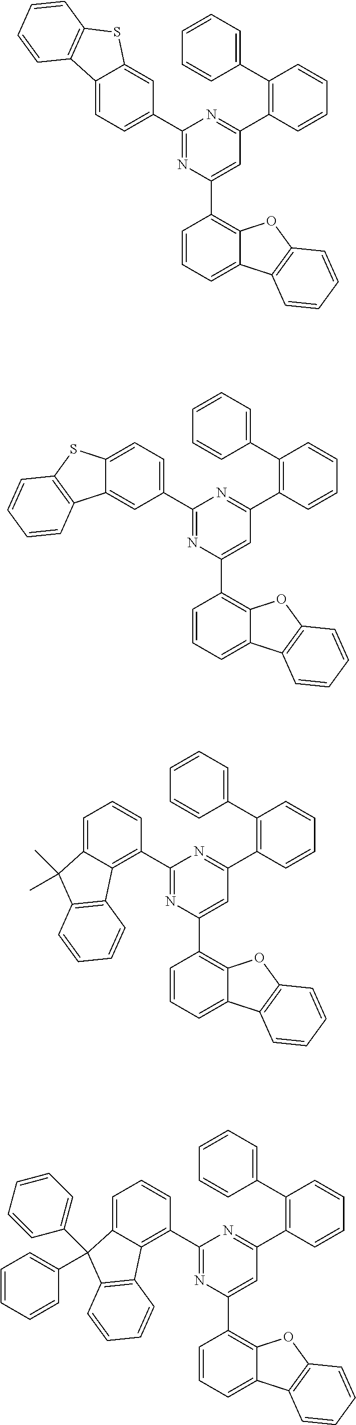 [SMILES: c1ccc(-c2ccccc2-c2cc(-c3cccc4c3oc3ccccc34)nc(-c3ccc4c(c3)sc3ccccc34)n2)cc1 ; c1ccc(-c2ccccc2-c2cc(-c3cccc4c3oc3ccccc34)nc(-c3ccc4sc5ccccc5c4c3)n2)cc1 ; CC1(C)c2ccccc2-c2c(-c3nc(-c4ccccc4-c4ccccc4)cc(-c4cccc5c4oc4ccccc45)n3)cccc21 ; c1ccc(-c2ccccc2-c2cc(-c3cccc4c3oc3ccccc34)nc(-c3cccc4c3-c3ccccc3C4(c3ccccc3)c3ccccc3)n2)cc1]

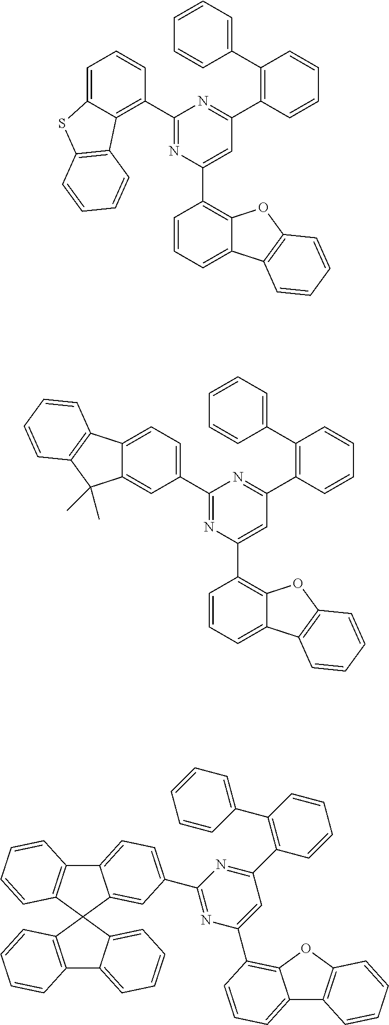 [SMILES: c1ccc(-c2ccccc2-c2cc(-c3cccc4c3oc3ccccc34)nc(-c3cccc4sc5ccccc5c34)n2)cc1 ; CC1(C)c2ccccc2-c2ccc(-c3nc(-c4ccccc4-c4ccccc4)cc(-c4cccc5c4oc4ccccc45)n3)cc21 ; c1ccc(-c2ccccc2-c2cc(-c3cccc4c3oc3ccccc34)nc(-c3ccc4c(c3)C3(c5ccccc5-c5ccccc53)c3ccccc3-4)n2)cc1]

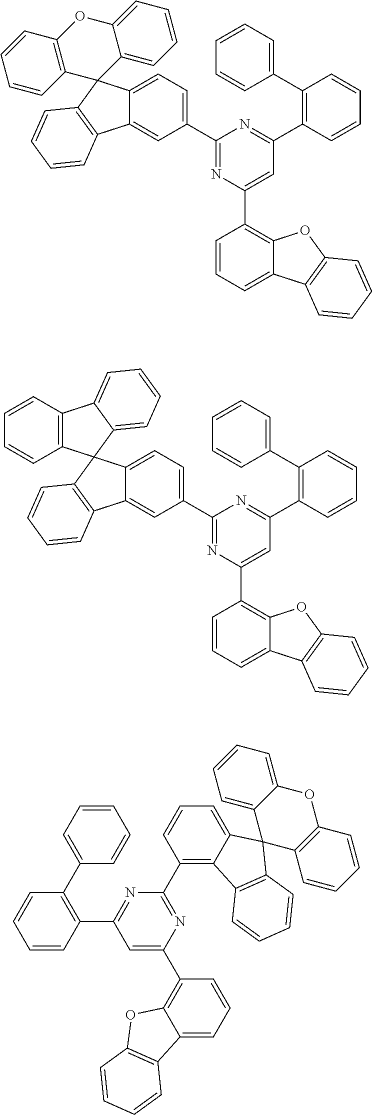 [SMILES: c1ccc(-c2ccccc2-c2cc(-c3cccc4c3oc3ccccc34)nc(-c3ccc4c(c3)-c3ccccc3C43c4ccccc4Oc4ccccc43)n2)cc1 ; c1ccc(-c2ccccc2-c2cc(-c3cccc4c3oc3ccccc34)nc(-c3ccc4c(c3)-c3ccccc3C43c4ccccc4-c4ccccc43)n2)cc1 ; c1ccc(-c2ccccc2-c2cc(-c3cccc4c3oc3ccccc34)nc(-c3cccc4c3-c3ccccc3C43c4ccccc4Oc4ccccc43)n2)cc1]

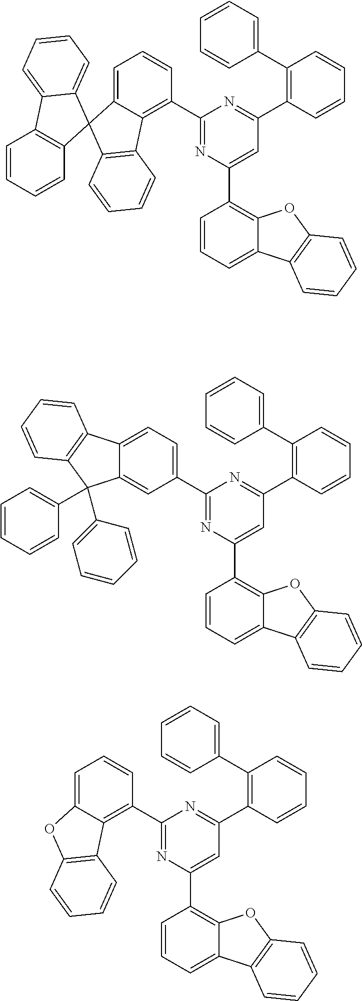 [SMILES: c1ccc(-c2ccccc2-c2cc(-c3cccc4c3oc3ccccc34)nc(-c3cccc4c3-c3ccccc3C43c4ccccc4-c4ccccc43)n2)cc1 ; c1ccc(-c2ccccc2-c2cc(-c3cccc4c3oc3ccccc34)nc(-c3ccc4c(c3)C(c3ccccc3)(c3ccccc3)c3ccccc3-4)n2)cc1 ; c1ccc(-c2ccccc2-c2cc(-c3cccc4c3oc3ccccc34)nc(-c3cccc4oc5ccccc5c34)n2)cc1]

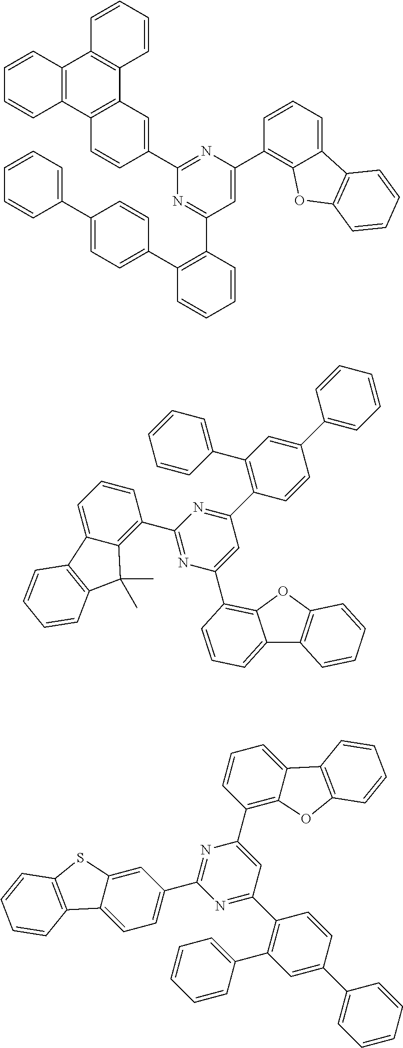 [SMILES: c1ccc(-c2ccc(-c3ccccc3-c3cc(-c4cccc5c4oc4ccccc45)nc(-c4ccc5c6ccccc6c6ccccc6c5c4)n3)cc2)cc1 ; CC1(C)c2ccccc2-c2cccc(-c3nc(-c4ccc(-c5ccccc5)cc4-c4ccccc4)cc(-c4cccc5c4oc4ccccc45)n3)c21 ; c1ccc(-c2ccc(-c3cc(-c4cccc5c4oc4ccccc45)nc(-c4ccc5c(c4)sc4ccccc45)n3)c(-c3ccccc3)c2)cc1]

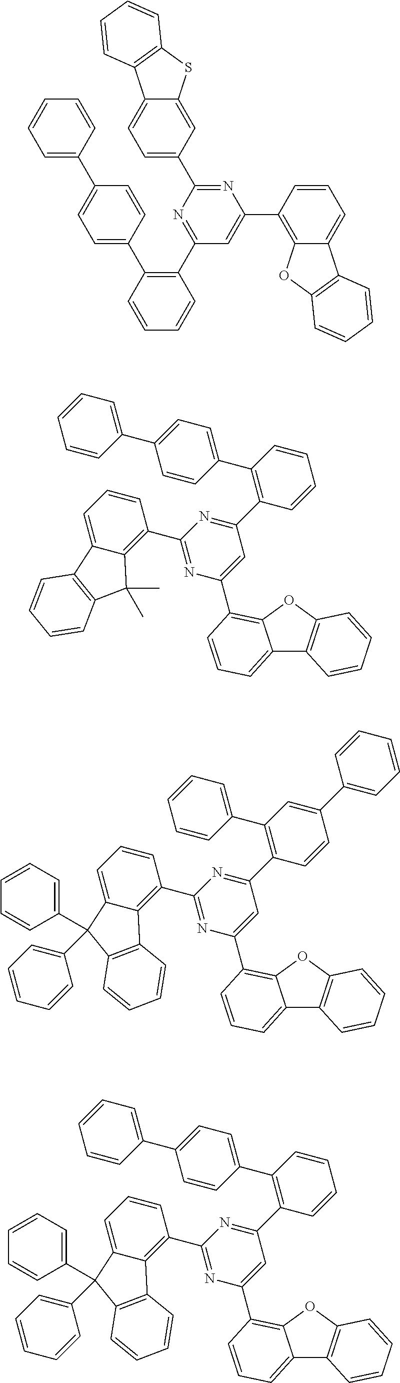 [SMILES: c1ccc(-c2ccc(-c3ccccc3-c3cc(-c4cccc5c4oc4ccccc45)nc(-c4ccc5c(c4)sc4ccccc45)n3)cc2)cc1 ; CC1(C)c2ccccc2-c2cccc(-c3nc(-c4ccccc4-c4ccc(-c5ccccc5)cc4)cc(-c4cccc5c4oc4ccccc45)n3)c21 ; c1ccc(-c2ccc(-c3cc(-c4cccc5c4oc4ccccc45)nc(-c4cccc5c4-c4ccccc4C5(c4ccccc4)c4ccccc4)n3)c(-c3ccccc3)c2)cc1 ; c1ccc(-c2ccc(-c3ccccc3-c3cc(-c4cccc5c4oc4ccccc45)nc(-c4cccc5c4-c4ccccc4C5(c4ccccc4)c4ccccc4)n3)cc2)cc1]

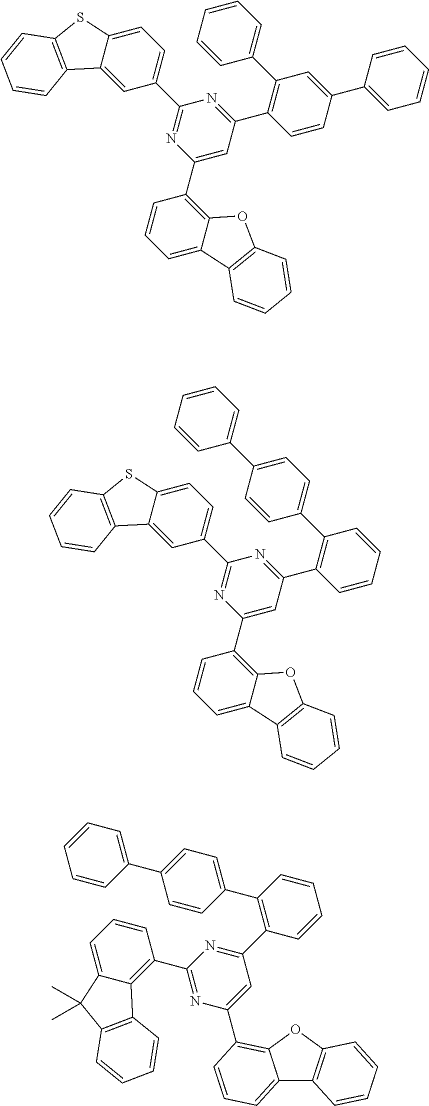 [SMILES: c1ccc(-c2ccc(-c3cc(-c4cccc5c4oc4ccccc45)nc(-c4ccc5sc6ccccc6c5c4)n3)c(-c3ccccc3)c2)cc1 ; c1ccc(-c2ccc(-c3ccccc3-c3cc(-c4cccc5c4oc4ccccc45)nc(-c4ccc5sc6ccccc6c5c4)n3)cc2)cc1 ; CC1(C)c2ccccc2-c2c(-c3nc(-c4ccccc4-c4ccc(-c5ccccc5)cc4)cc(-c4cccc5c4oc4ccccc45)n3)cccc21]

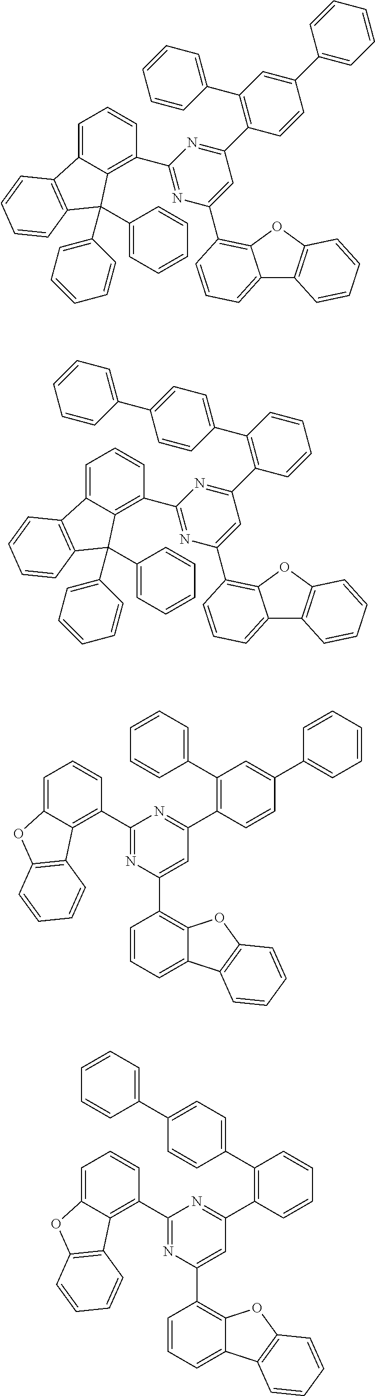 [SMILES: c1ccc(-c2ccc(-c3cc(-c4cccc5c4oc4ccccc45)nc(-c4cccc5c4C(c4ccccc4)(c4ccccc4)c4ccccc4-5)n3)c(-c3ccccc3)c2)cc1 ; c1ccc(-c2ccc(-c3ccccc3-c3cc(-c4cccc5c4oc4ccccc45)nc(-c4cccc5c4C(c4ccccc4)(c4ccccc4)c4ccccc4-5)n3)cc2)cc1 ; c1ccc(-c2ccc(-c3cc(-c4cccc5c4oc4ccccc45)nc(-c4cccc5oc6ccccc6c45)n3)c(-c3ccccc3)c2)cc1 ; c1ccc(-c2ccc(-c3ccccc3-c3cc(-c4cccc5c4oc4ccccc45)nc(-c4cccc5oc6ccccc6c45)n3)cc2)cc1]

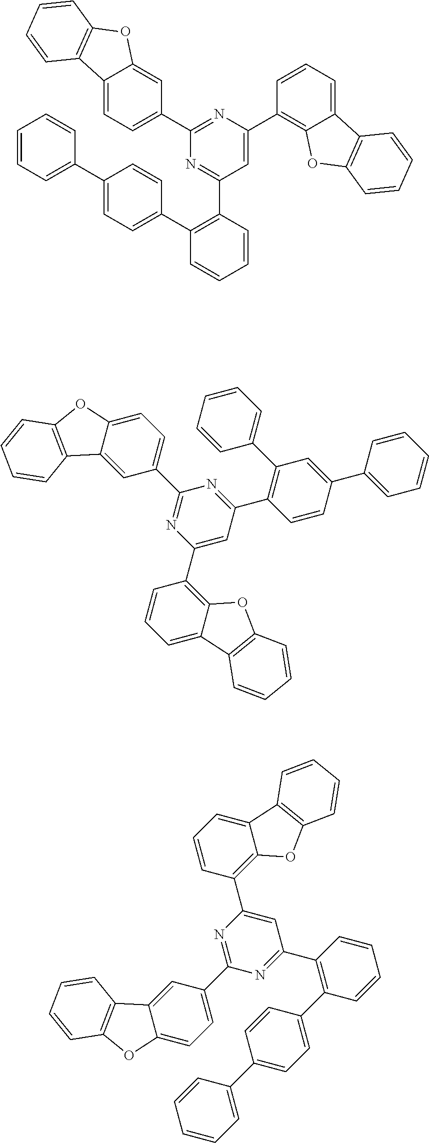 [SMILES: c1ccc(-c2ccc(-c3ccccc3-c3cc(-c4cccc5c4oc4ccccc45)nc(-c4ccc5c(c4)oc4ccccc45)n3)cc2)cc1 ; c1ccc(-c2ccc(-c3cc(-c4cccc5c4oc4ccccc45)nc(-c4ccc5oc6ccccc6c5c4)n3)c(-c3ccccc3)c2)cc1 ; c1ccc(-c2ccc(-c3ccccc3-c3cc(-c4cccc5c4oc4ccccc45)nc(-c4ccc5oc6ccccc6c5c4)n3)cc2)cc1]

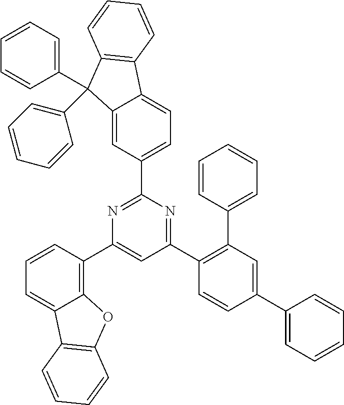 [SMILES: c1ccc(-c2ccc(-c3cc(-c4cccc5c4oc4ccccc45)nc(-c4ccc5c(c4)C(c4ccccc4)(c4ccccc4)c4ccccc4-5)n3)c(-c3ccccc3)c2)cc1]

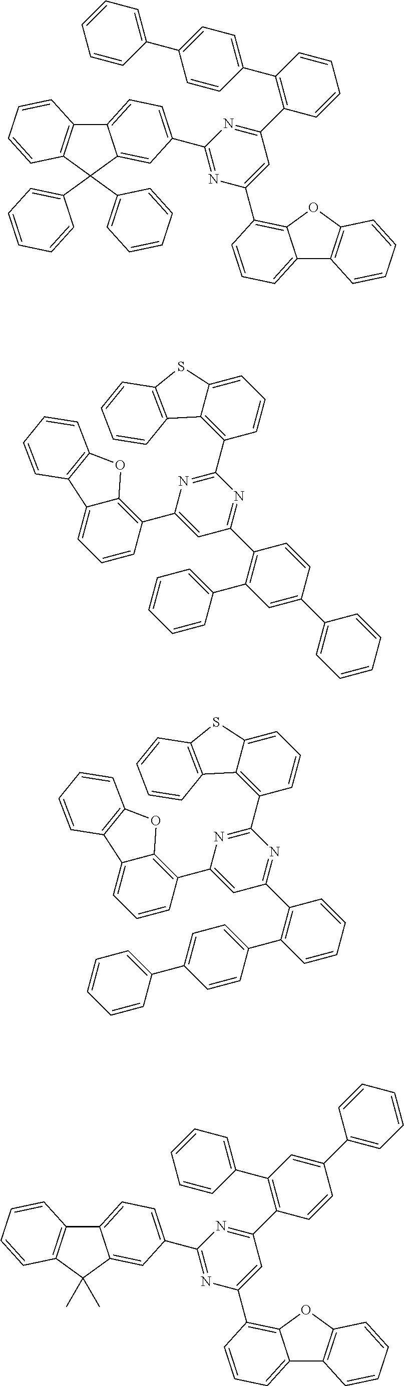 [SMILES: c1ccc(-c2ccc(-c3ccccc3-c3cc(-c4cccc5c4oc4ccccc45)nc(-c4ccc5c(c4)C(c4ccccc4)(c4ccccc4)c4ccccc4-5)n3)cc2)cc1 ; c1ccc(-c2ccc(-c3cc(-c4cccc5c4oc4ccccc45)nc(-c4cccc5sc6ccccc6c45)n3)c(-c3ccccc3)c2)cc1 ; c1ccc(-c2ccc(-c3ccccc3-c3cc(-c4cccc5c4oc4ccccc45)nc(-c4cccc5sc6ccccc6c45)n3)cc2)cc1 ; CC1(C)c2ccccc2-c2ccc(-c3nc(-c4ccc(-c5ccccc5)cc4-c4ccccc4)cc(-c4cccc5c4oc4ccccc45)n3)cc21]

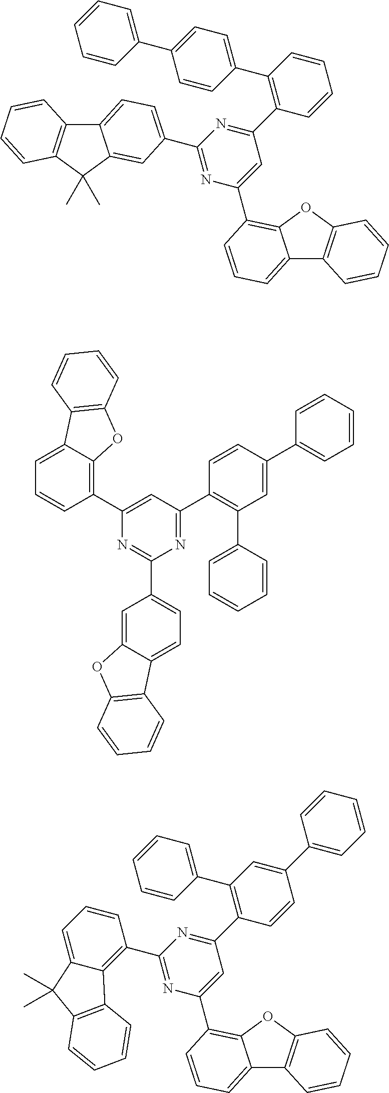 [SMILES: CC1(C)c2ccccc2-c2ccc(-c3nc(-c4ccccc4-c4ccc(-c5ccccc5)cc4)cc(-c4cccc5c4oc4ccccc45)n3)cc21 ; c1ccc(-c2ccc(-c3cc(-c4cccc5c4oc4ccccc45)nc(-c4ccc5c(c4)oc4ccccc45)n3)c(-c3ccccc3)c2)cc1 ; CC1(C)c2ccccc2-c2c(-c3nc(-c4ccc(-c5ccccc5)cc4-c4ccccc4)cc(-c4cccc5c4oc4ccccc45)n3)cccc21]

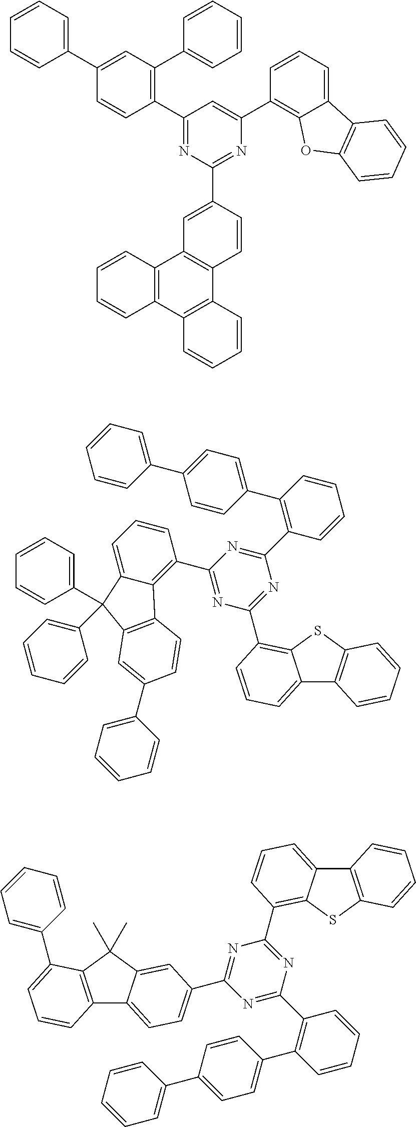 [SMILES: c1ccc(-c2ccc(-c3cc(-c4cccc5c4oc4ccccc45)nc(-c4ccc5c6ccccc6c6ccccc6c5c4)n3)c(-c3ccccc3)c2)cc1 ; c1ccc(-c2ccc(-c3ccccc3-c3nc(-c4cccc5c4-c4ccc(-c6ccccc6)cc4C5(c4ccccc4)c4ccccc4)nc(-c4cccc5c4sc4ccccc45)n3)cc2)cc1 ; CC1(C)c2cc(-c3nc(-c4ccccc4-c4ccc(-c5ccccc5)cc4)nc(-c4cccc5c4sc4ccccc45)n3)ccc2-c2cccc(-c3ccccc3)c21]

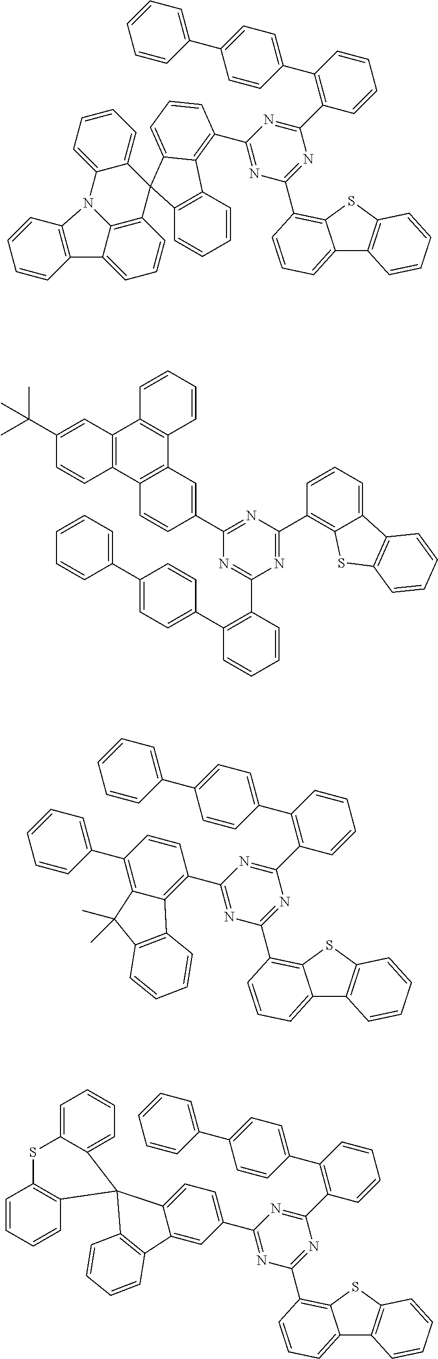 [SMILES: c1ccc(-c2ccc(-c3ccccc3-c3nc(-c4cccc5c4-c4ccccc4C54c5ccccc5-n5c6ccccc6c6cccc4c65)nc(-c4cccc5c4sc4ccccc45)n3)cc2)cc1 ; CC(C)(C)c1ccc2c3ccc(-c4nc(-c5ccccc5-c5ccc(-c6ccccc6)cc5)nc(-c5cccc6c5sc5ccccc56)n4)cc3c3ccccc3c2c1 ; CC1(C)c2ccccc2-c2c(-c3nc(-c4ccccc4-c4ccc(-c5ccccc5)cc4)nc(-c4cccc5c4sc4ccccc45)n3)ccc(-c3ccccc3)c21 ; c1ccc(-c2ccc(-c3ccccc3-c3nc(-c4ccc5c(c4)-c4ccccc4C54c5ccccc5Sc5ccccc54)nc(-c4cccc5c4sc4ccccc45)n3)cc2)cc1]

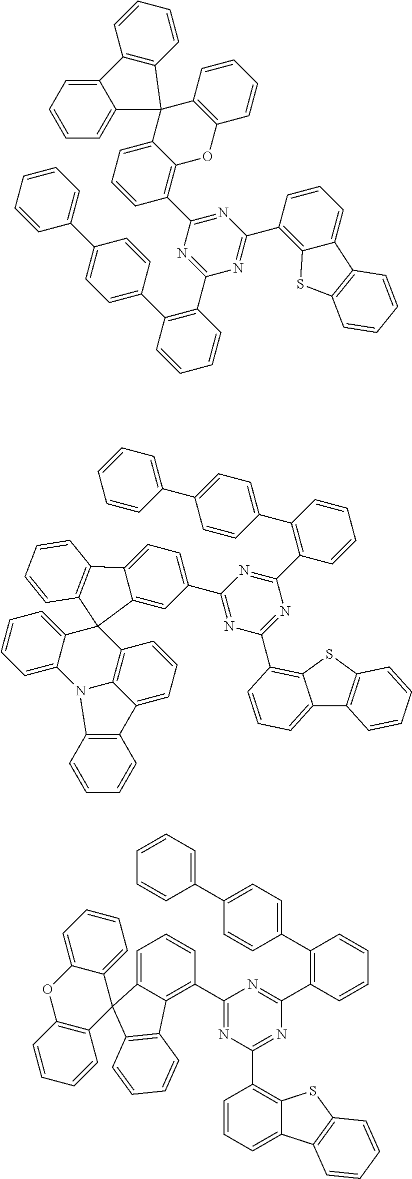 [SMILES: c1ccc(-c2ccc(-c3ccccc3-c3nc(-c4cccc5c4Oc4ccccc4C54c5ccccc5-c5ccccc54)nc(-c4cccc5c4sc4ccccc45)n3)cc2)cc1 ; c1ccc(-c2ccc(-c3ccccc3-c3nc(-c4ccc5c(c4)C4(c6ccccc6-5)c5ccccc5-n5c6ccccc6c6cccc4c65)nc(-c4cccc5c4sc4ccccc45)n3)cc2)cc1 ; c1ccc(-c2ccc(-c3ccccc3-c3nc(-c4cccc5c4-c4ccccc4C54c5ccccc5Oc5ccccc54)nc(-c4cccc5c4sc4ccccc45)n3)cc2)cc1]

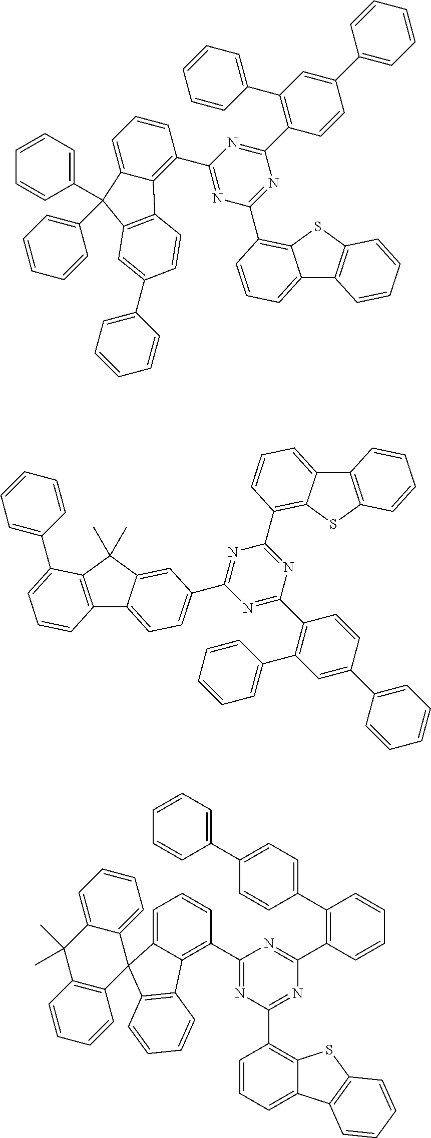 [SMILES: c1ccc(-c2ccc(-c3nc(-c4cccc5c4-c4ccc(-c6ccccc6)cc4C5(c4ccccc4)c4ccccc4)nc(-c4cccc5c4sc4ccccc45)n3)c(-c3ccccc3)c2)cc1 ; CC1(C)c2cc(-c3nc(-c4ccc(-c5ccccc5)cc4-c4ccccc4)nc(-c4cccc5c4sc4ccccc45)n3)ccc2-c2cccc(-c3ccccc3)c21 ; CC1(C)c2ccccc2C2(c3ccccc3-c3c(-c4nc(-c5ccccc5-c5ccc(-c6ccccc6)cc5)nc(-c5cccc6c5sc5ccccc56)n4)cccc32)c2ccccc21]

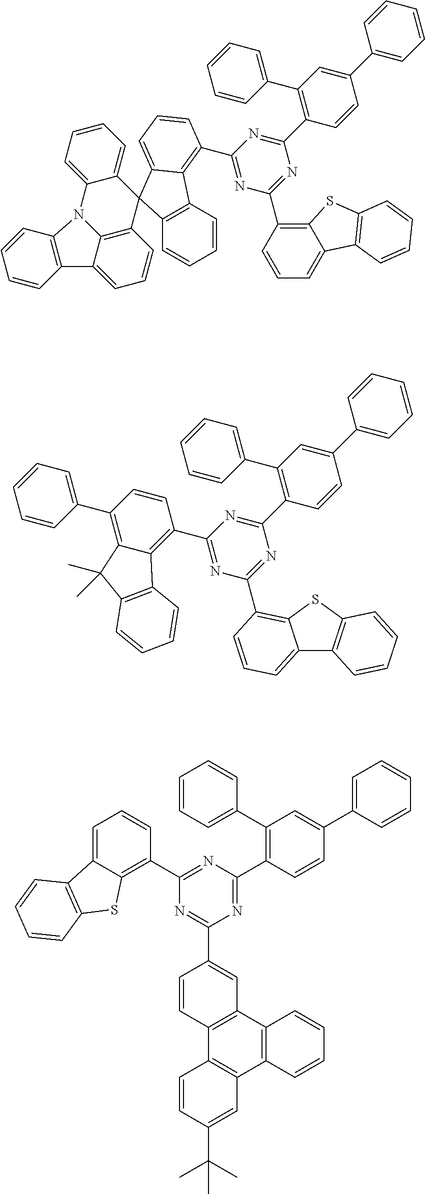 [SMILES: c1ccc(-c2ccc(-c3nc(-c4cccc5c4-c4ccccc4C54c5ccccc5-n5c6ccccc6c6cccc4c65)nc(-c4cccc5c4sc4ccccc45)n3)c(-c3ccccc3)c2)cc1 ; CC1(C)c2ccccc2-c2c(-c3nc(-c4ccc(-c5ccccc5)cc4-c4ccccc4)nc(-c4cccc5c4sc4ccccc45)n3)ccc(-c3ccccc3)c21 ; CC(C)(C)c1ccc2c3ccc(-c4nc(-c5ccc(-c6ccccc6)cc5-c5ccccc5)nc(-c5cccc6c5sc5ccccc56)n4)cc3c3ccccc3c2c1]

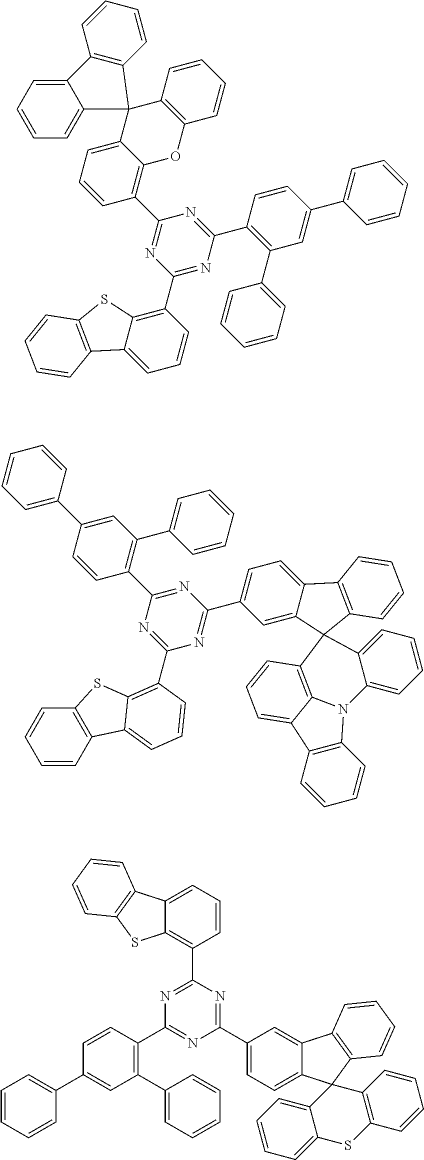 [SMILES: c1ccc(-c2ccc(-c3nc(-c4cccc5c4Oc4ccccc4C54c5ccccc5-c5ccccc54)nc(-c4cccc5c4sc4ccccc45)n3)c(-c3ccccc3)c2)cc1 ; c1ccc(-c2ccc(-c3nc(-c4ccc5c(c4)C4(c6ccccc6-5)c5ccccc5-n5c6ccccc6c6cccc4c65)nc(-c4cccc5c4sc4ccccc45)n3)c(-c3ccccc3)c2)cc1 ; c1ccc(-c2ccc(-c3nc(-c4ccc5c(c4)-c4ccccc4C54c5ccccc5Sc5ccccc54)nc(-c4cccc5c4sc4ccccc45)n3)c(-c3ccccc3)c2)cc1]

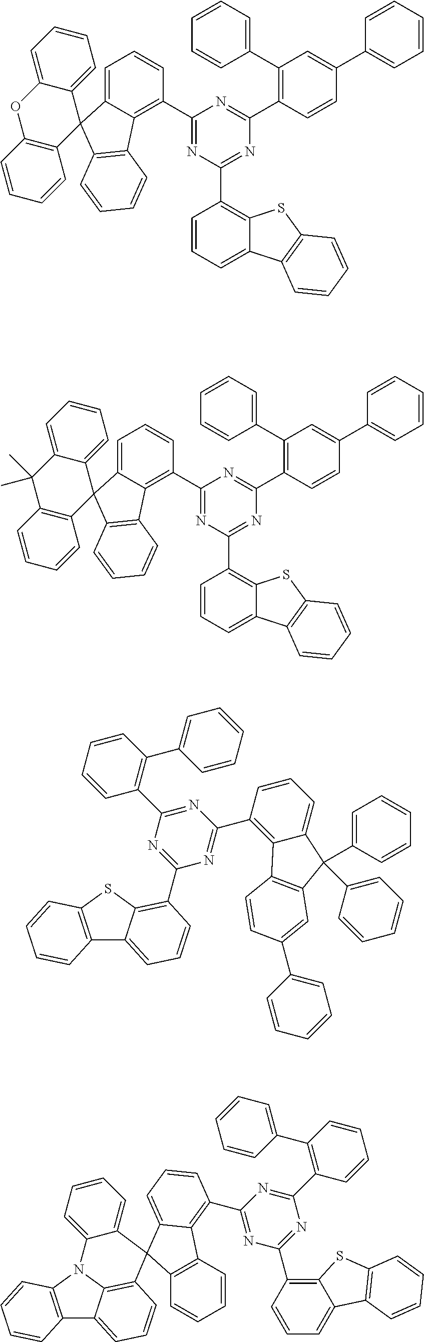 [SMILES: c1ccc(-c2ccc(-c3nc(-c4cccc5c4-c4ccccc4C54c5ccccc5Oc5ccccc54)nc(-c4cccc5c4sc4ccccc45)n3)c(-c3ccccc3)c2)cc1 ; CC1(C)c2ccccc2C2(c3ccccc3-c3c(-c4nc(-c5ccc(-c6ccccc6)cc5-c5ccccc5)nc(-c5cccc6c5sc5ccccc56)n4)cccc32)c2ccccc21 ; c1ccc(-c2ccc3c(c2)C(c2ccccc2)(c2ccccc2)c2cccc(-c4nc(-c5ccccc5-c5ccccc5)nc(-c5cccc6c5sc5ccccc56)n4)c2-3)cc1 ; c1ccc(-c2ccccc2-c2nc(-c3cccc4c3-c3ccccc3C43c4ccccc4-n4c5ccccc5c5cccc3c54)nc(-c3cccc4c3sc3ccccc34)n2)cc1]

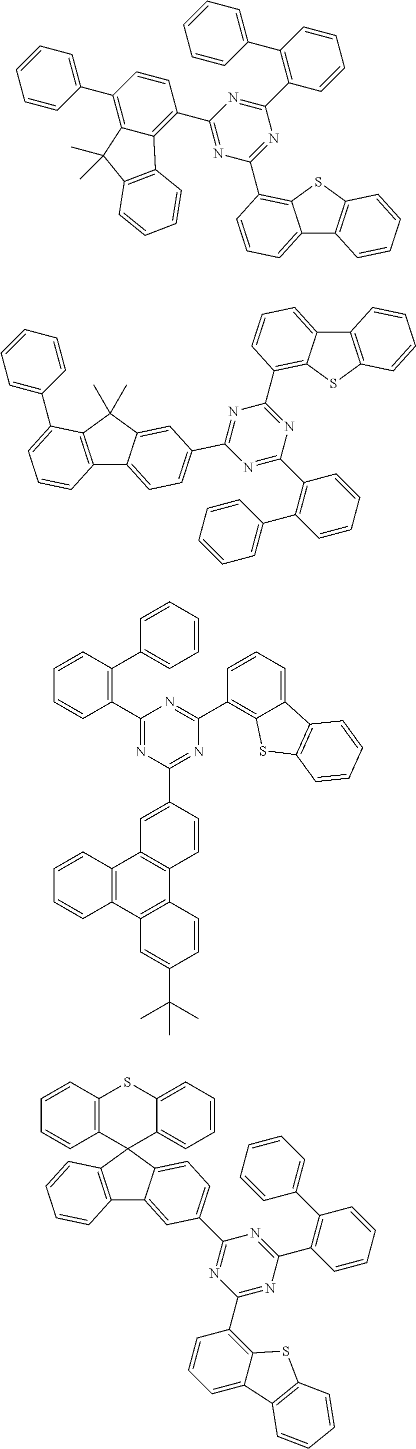 [SMILES: CC1(C)c2ccccc2-c2c(-c3nc(-c4ccccc4-c4ccccc4)nc(-c4cccc5c4sc4ccccc45)n3)ccc(-c3ccccc3)c21 ; CC1(C)c2cc(-c3nc(-c4ccccc4-c4ccccc4)nc(-c4cccc5c4sc4ccccc45)n3)ccc2-c2cccc(-c3ccccc3)c21 ; CC(C)(C)c1ccc2c3ccc(-c4nc(-c5ccccc5-c5ccccc5)nc(-c5cccc6c5sc5ccccc56)n4)cc3c3ccccc3c2c1 ; c1ccc(-c2ccccc2-c2nc(-c3ccc4c(c3)-c3ccccc3C43c4ccccc4Sc4ccccc43)nc(-c3cccc4c3sc3ccccc34)n2)cc1]

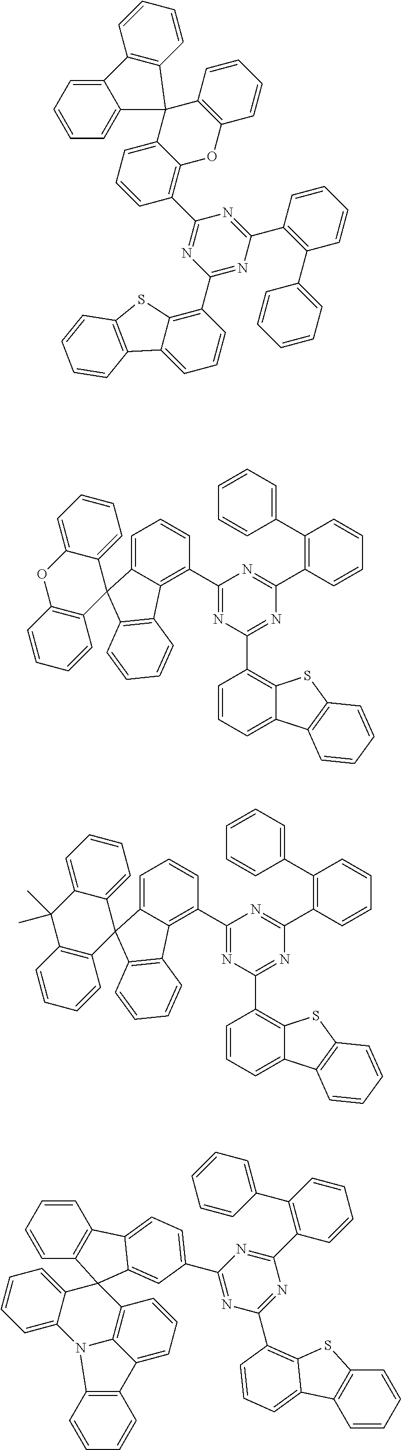 [SMILES: c1ccc(-c2ccccc2-c2nc(-c3cccc4c3Oc3ccccc3C43c4ccccc4-c4ccccc43)nc(-c3cccc4c3sc3ccccc34)n2)cc1 ; c1ccc(-c2ccccc2-c2nc(-c3cccc4c3-c3ccccc3C43c4ccccc4Oc4ccccc43)nc(-c3cccc4c3sc3ccccc34)n2)cc1 ; CC1(C)c2ccccc2C2(c3ccccc3-c3c(-c4nc(-c5ccccc5-c5ccccc5)nc(-c5cccc6c5sc5ccccc56)n4)cccc32)c2ccccc21 ; c1ccc(-c2ccccc2-c2nc(-c3ccc4c(c3)C3(c5ccccc5-4)c4ccccc4-n4c5ccccc5c5cccc3c54)nc(-c3cccc4c3sc3ccccc34)n2)cc1]

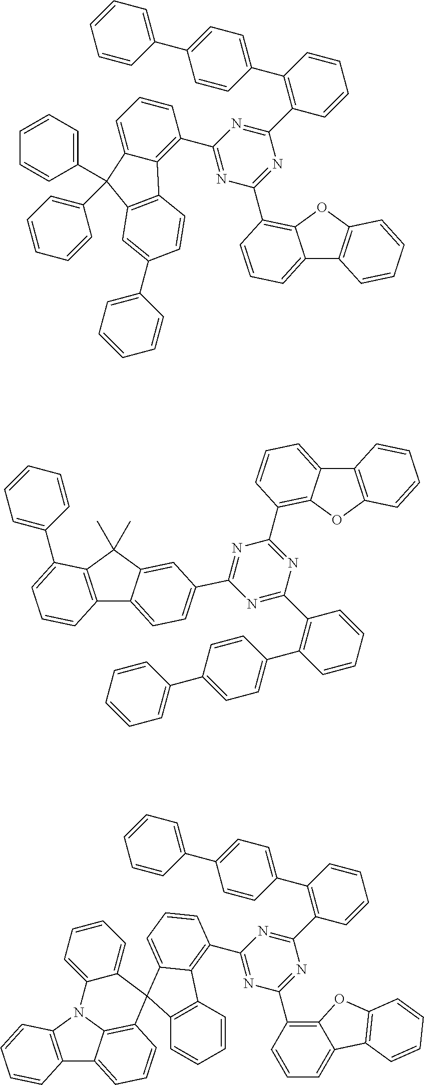 [SMILES: c1ccc(-c2ccc(-c3ccccc3-c3nc(-c4cccc5c4-c4ccc(-c6ccccc6)cc4C5(c4ccccc4)c4ccccc4)nc(-c4cccc5c4oc4ccccc45)n3)cc2)cc1 ; CC1(C)c2cc(-c3nc(-c4ccccc4-c4ccc(-c5ccccc5)cc4)nc(-c4cccc5c4oc4ccccc45)n3)ccc2-c2cccc(-c3ccccc3)c21 ; c1ccc(-c2ccc(-c3ccccc3-c3nc(-c4cccc5c4-c4ccccc4C54c5ccccc5-n5c6ccccc6c6cccc4c65)nc(-c4cccc5c4oc4ccccc45)n3)cc2)cc1]

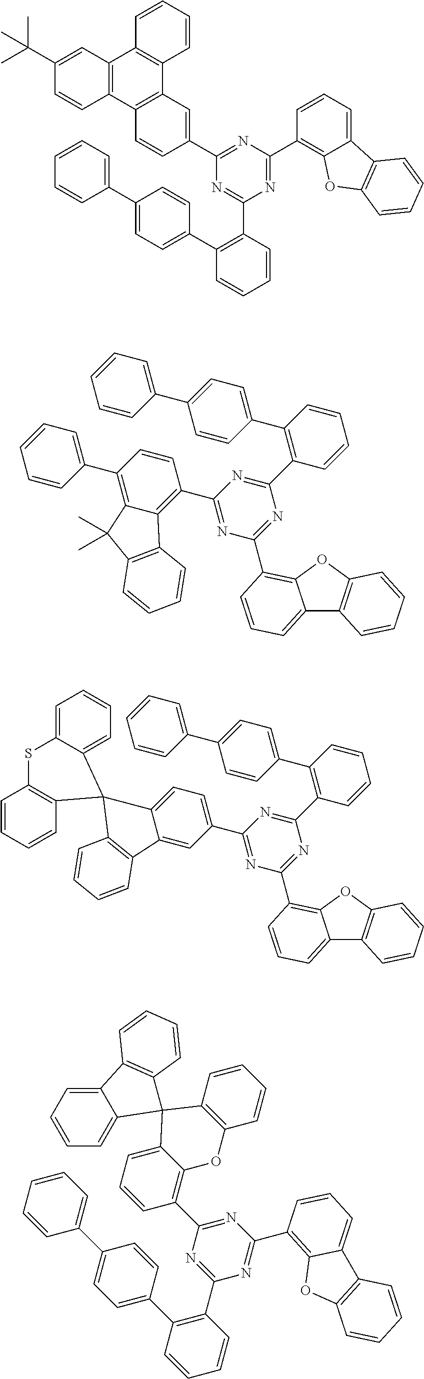 [SMILES: CC(C)(C)c1ccc2c3ccc(-c4nc(-c5ccccc5-c5ccc(-c6ccccc6)cc5)nc(-c5cccc6c5oc5ccccc56)n4)cc3c3ccccc3c2c1 ; CC1(C)c2ccccc2-c2c(-c3nc(-c4ccccc4-c4ccc(-c5ccccc5)cc4)nc(-c4cccc5c4oc4ccccc45)n3)ccc(-c3ccccc3)c21 ; c1ccc(-c2ccc(-c3ccccc3-c3nc(-c4ccc5c(c4)-c4ccccc4C54c5ccccc5Sc5ccccc54)nc(-c4cccc5c4oc4ccccc45)n3)cc2)cc1 ; c1ccc(-c2ccc(-c3ccccc3-c3nc(-c4cccc5c4Oc4ccccc4C54c5ccccc5-c5ccccc54)nc(-c4cccc5c4oc4ccccc45)n3)cc2)cc1]

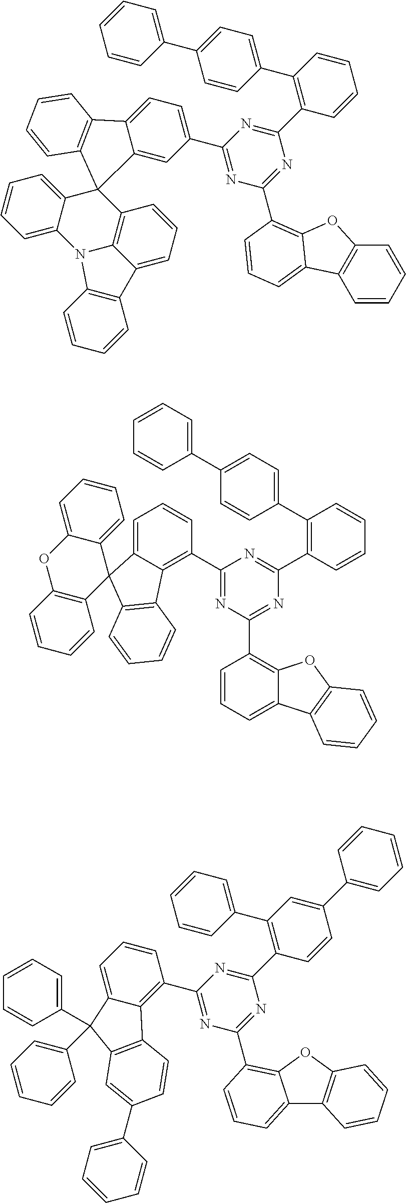 [SMILES: c1ccc(-c2ccc(-c3ccccc3-c3nc(-c4ccc5c(c4)C4(c6ccccc6-5)c5ccccc5-n5c6ccccc6c6cccc4c65)nc(-c4cccc5c4oc4ccccc45)n3)cc2)cc1 ; c1ccc(-c2ccc(-c3ccccc3-c3nc(-c4cccc5c4-c4ccccc4C54c5ccccc5Oc5ccccc54)nc(-c4cccc5c4oc4ccccc45)n3)cc2)cc1 ; c1ccc(-c2ccc(-c3nc(-c4cccc5c4-c4ccc(-c6ccccc6)cc4C5(c4ccccc4)c4ccccc4)nc(-c4cccc5c4oc4ccccc45)n3)c(-c3ccccc3)c2)cc1]

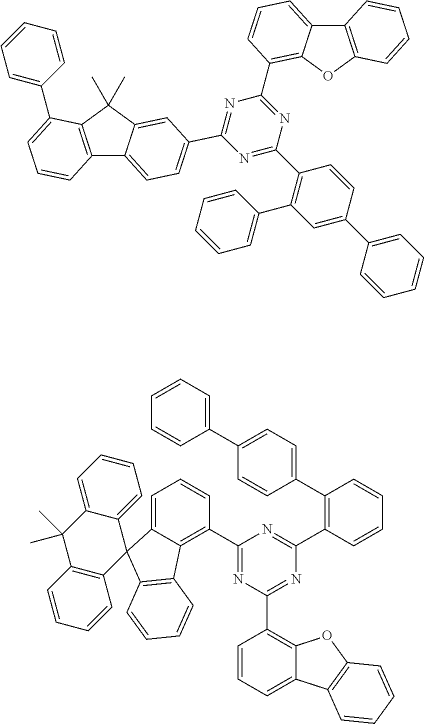 [SMILES: CC1(C)c2cc(-c3nc(-c4ccc(-c5ccccc5)cc4-c4ccccc4)nc(-c4cccc5c4oc4ccccc45)n3)ccc2-c2cccc(-c3ccccc3)c21 ; CC1(C)c2ccccc2C2(c3ccccc3-c3c(-c4nc(-c5ccccc5-c5ccc(-c6ccccc6)cc5)nc(-c5cccc6c5oc5ccccc56)n4)cccc32)c2ccccc21]

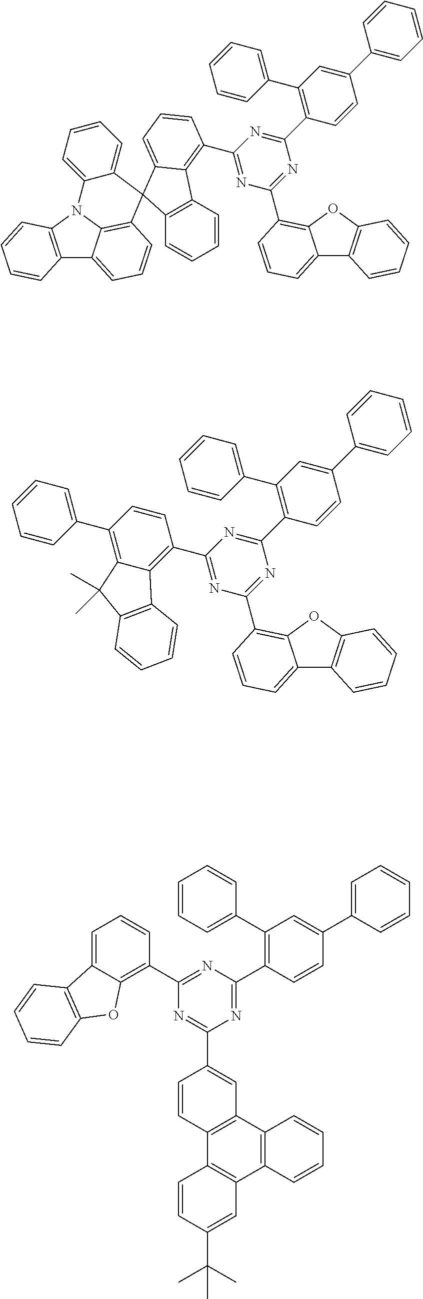 [SMILES: c1ccc(-c2ccc(-c3nc(-c4cccc5c4-c4ccccc4C54c5ccccc5-n5c6ccccc6c6cccc4c65)nc(-c4cccc5c4oc4ccccc45)n3)c(-c3ccccc3)c2)cc1 ; CC1(C)c2ccccc2-c2c(-c3nc(-c4ccc(-c5ccccc5)cc4-c4ccccc4)nc(-c4cccc5c4oc4ccccc45)n3)ccc(-c3ccccc3)c21 ; CC(C)(C)c1ccc2c3ccc(-c4nc(-c5ccc(-c6ccccc6)cc5-c5ccccc5)nc(-c5cccc6c5oc5ccccc56)n4)cc3c3ccccc3c2c1]

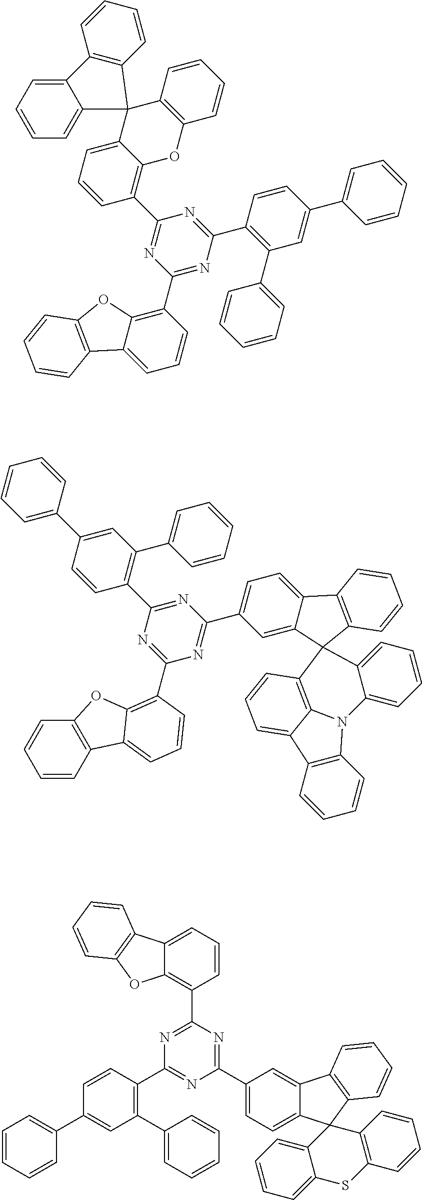 [SMILES: c1ccc(-c2ccc(-c3nc(-c4cccc5c4Oc4ccccc4C54c5ccccc5-c5ccccc54)nc(-c4cccc5c4oc4ccccc45)n3)c(-c3ccccc3)c2)cc1 ; c1ccc(-c2ccc(-c3nc(-c4ccc5c(c4)C4(c6ccccc6-5)c5ccccc5-n5c6ccccc6c6cccc4c65)nc(-c4cccc5c4oc4ccccc45)n3)c(-c3ccccc3)c2)cc1 ; c1ccc(-c2ccc(-c3nc(-c4ccc5c(c4)-c4ccccc4C54c5ccccc5Sc5ccccc54)nc(-c4cccc5c4oc4ccccc45)n3)c(-c3ccccc3)c2)cc1]

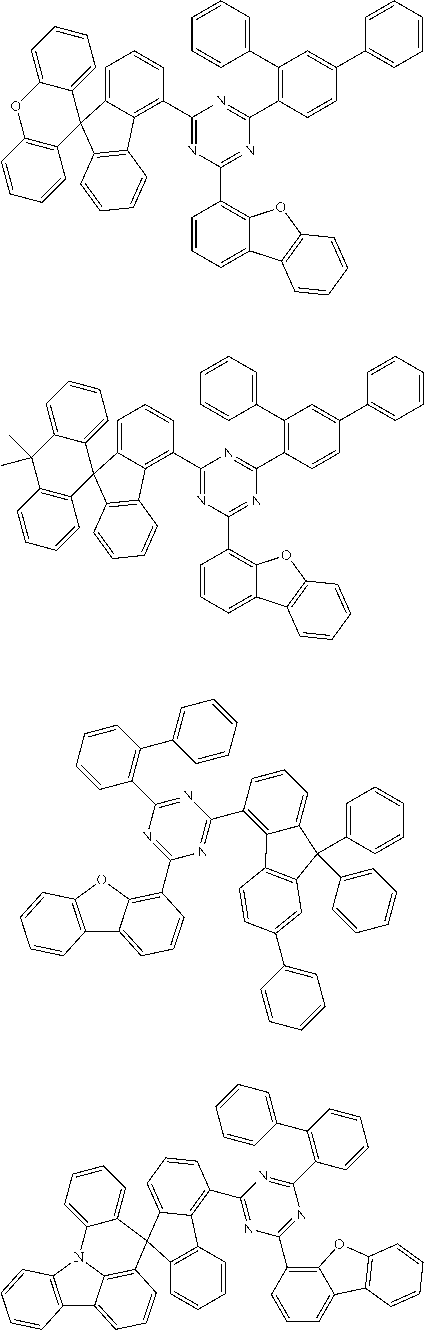 [SMILES: c1ccc(-c2ccc(-c3nc(-c4cccc5c4-c4ccccc4C54c5ccccc5Oc5ccccc54)nc(-c4cccc5c4oc4ccccc45)n3)c(-c3ccccc3)c2)cc1 ; CC1(C)c2ccccc2C2(c3ccccc3-c3c(-c4nc(-c5ccc(-c6ccccc6)cc5-c5ccccc5)nc(-c5cccc6c5oc5ccccc56)n4)cccc32)c2ccccc21 ; c1ccc(-c2ccc3c(c2)C(c2ccccc2)(c2ccccc2)c2cccc(-c4nc(-c5ccccc5-c5ccccc5)nc(-c5cccc6c5oc5ccccc56)n4)c2-3)cc1 ; c1ccc(-c2ccccc2-c2nc(-c3cccc4c3-c3ccccc3C43c4ccccc4-n4c5ccccc5c5cccc3c54)nc(-c3cccc4c3oc3ccccc34)n2)cc1]

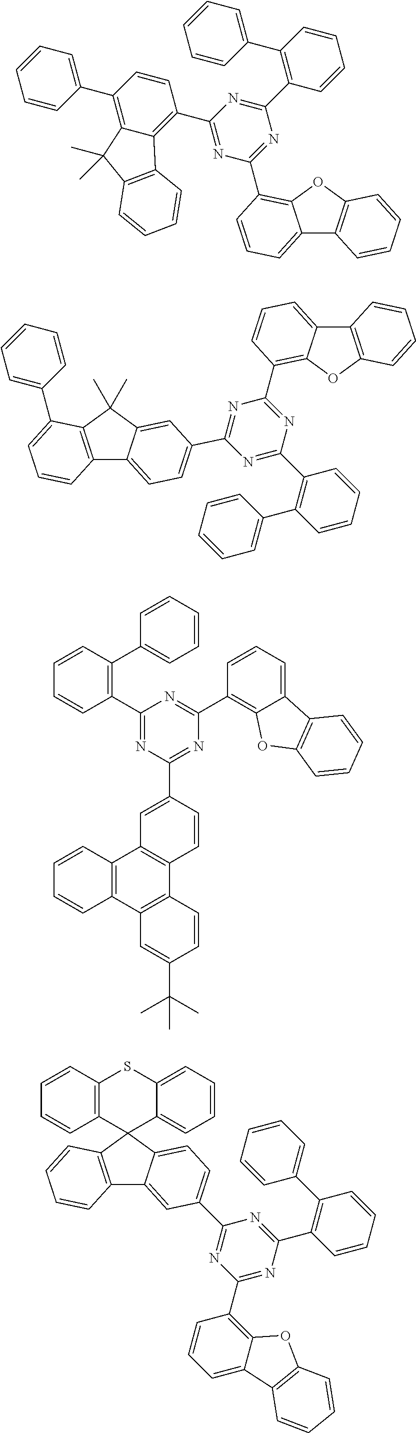

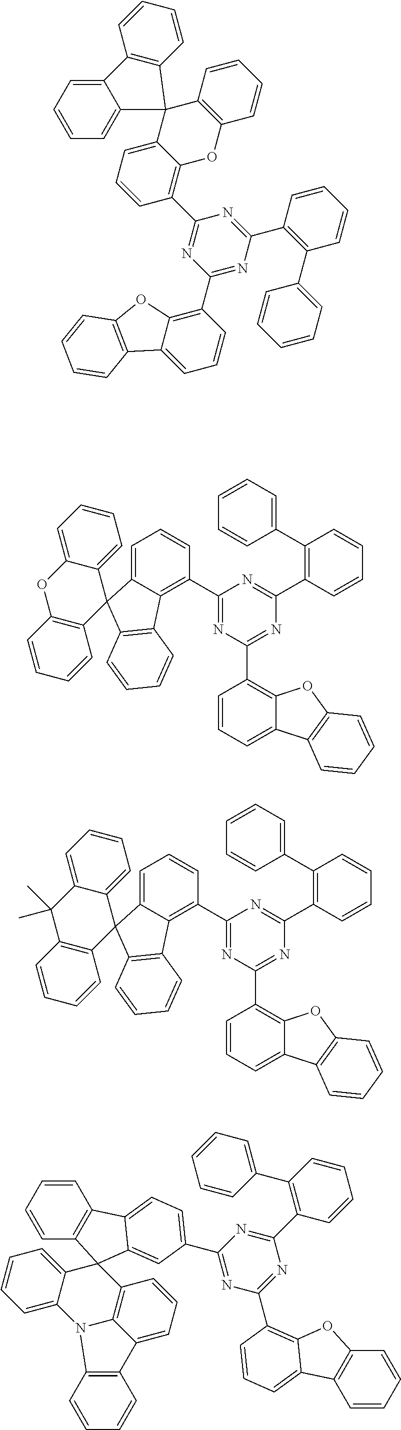 [SMILES: c1ccc(-c2ccccc2-c2nc(-c3cccc4c3Oc3ccccc3C43c4ccccc4-c4ccccc43)nc(-c3cccc4c3oc3ccccc34)n2)cc1 ; c1ccc(-c2ccccc2-c2nc(-c3cccc4c3-c3ccccc3C43c4ccccc4Oc4ccccc43)nc(-c3cccc4c3oc3ccccc34)n2)cc1 ; CC1(C)c2ccccc2C2(c3ccccc3-c3c(-c4nc(-c5ccccc5-c5ccccc5)nc(-c5cccc6c5oc5ccccc56)n4)cccc32)c2ccccc21 ; c1ccc(-c2ccccc2-c2nc(-c3ccc4c(c3)C3(c5ccccc5-4)c4ccccc4-n4c5ccccc5c5cccc3c54)nc(-c3cccc4c3oc3ccccc34)n2)cc1]

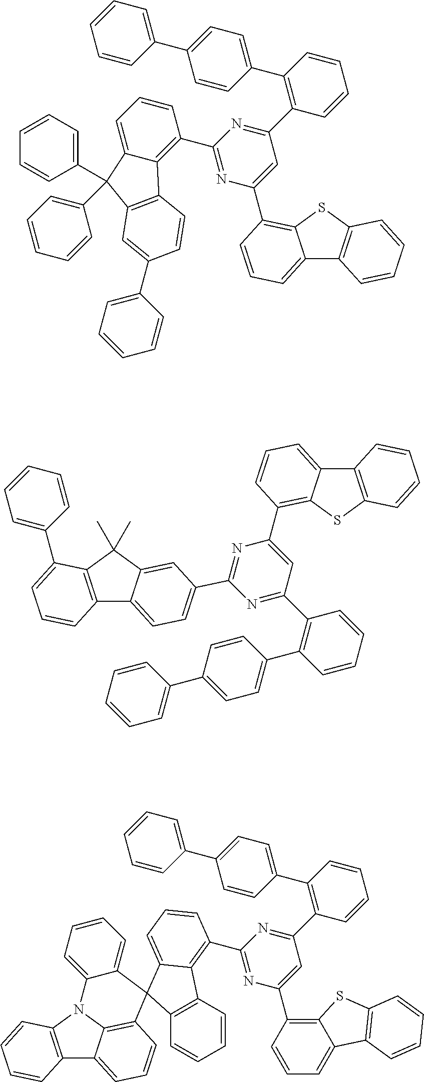 [SMILES: c1ccc(-c2ccc(-c3ccccc3-c3cc(-c4cccc5c4sc4ccccc45)nc(-c4cccc5c4-c4ccc(-c6ccccc6)cc4C5(c4ccccc4)c4ccccc4)n3)cc2)cc1 ; CC1(C)c2cc(-c3nc(-c4ccccc4-c4ccc(-c5ccccc5)cc4)cc(-c4cccc5c4sc4ccccc45)n3)ccc2-c2cccc(-c3ccccc3)c21 ; c1ccc(-c2ccc(-c3ccccc3-c3cc(-c4cccc5c4sc4ccccc45)nc(-c4cccc5c4-c4ccccc4C54c5ccccc5-n5c6ccccc6c6cccc4c65)n3)cc2)cc1]

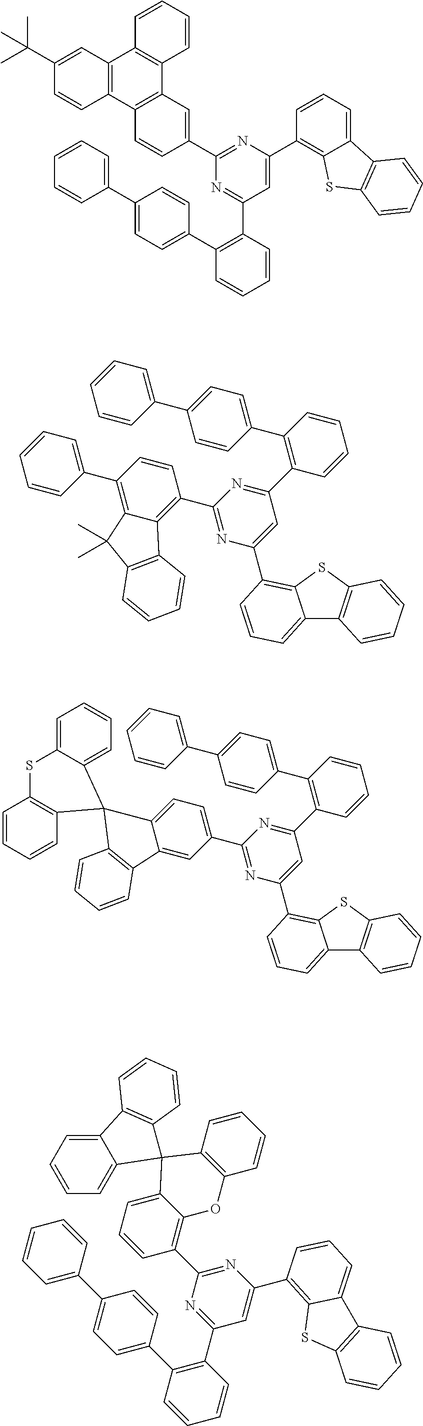 [SMILES: CC(C)(C)c1ccc2c3ccc(-c4nc(-c5ccccc5-c5ccc(-c6ccccc6)cc5)cc(-c5cccc6c5sc5ccccc56)n4)cc3c3ccccc3c2c1 ; CC1(C)c2ccccc2-c2c(-c3nc(-c4ccccc4-c4ccc(-c5ccccc5)cc4)cc(-c4cccc5c4sc4ccccc45)n3)ccc(-c3ccccc3)c21 ; c1ccc(-c2ccc(-c3ccccc3-c3cc(-c4cccc5c4sc4ccccc45)nc(-c4ccc5c(c4)-c4ccccc4C54c5ccccc5Sc5ccccc54)n3)cc2)cc1 ; c1ccc(-c2ccc(-c3ccccc3-c3cc(-c4cccc5c4sc4ccccc45)nc(-c4cccc5c4Oc4ccccc4C54c5ccccc5-c5ccccc54)n3)cc2)cc1]

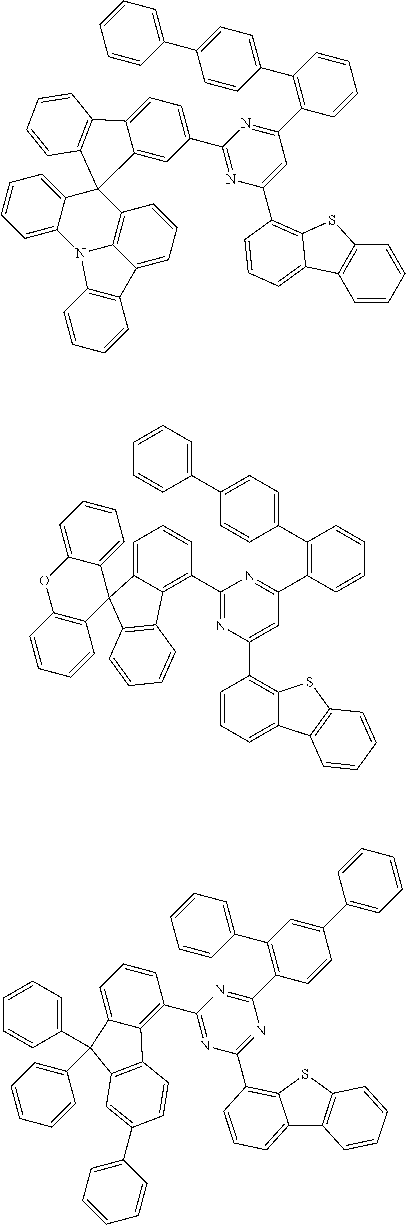 [SMILES: c1ccc(-c2ccc(-c3ccccc3-c3cc(-c4cccc5c4sc4ccccc45)nc(-c4ccc5c(c4)C4(c6ccccc6-5)c5ccccc5-n5c6ccccc6c6cccc4c65)n3)cc2)cc1 ; c1ccc(-c2ccc(-c3ccccc3-c3cc(-c4cccc5c4sc4ccccc45)nc(-c4cccc5c4-c4ccccc4C54c5ccccc5Oc5ccccc54)n3)cc2)cc1 ; c1ccc(-c2ccc(-c3nc(-c4cccc5c4-c4ccc(-c6ccccc6)cc4C5(c4ccccc4)c4ccccc4)nc(-c4cccc5c4sc4ccccc45)n3)c(-c3ccccc3)c2)cc1]

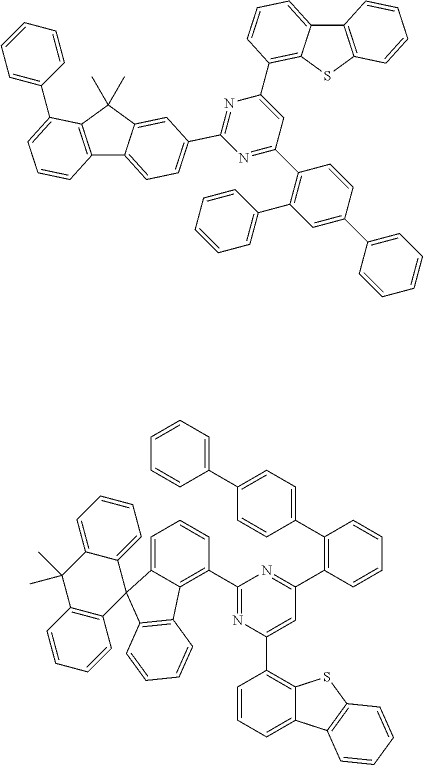 [SMILES: CC1(C)c2cc(-c3nc(-c4ccc(-c5ccccc5)cc4-c4ccccc4)cc(-c4cccc5c4sc4ccccc45)n3)ccc2-c2cccc(-c3ccccc3)c21 ; CC1(C)c2ccccc2C2(c3ccccc3-c3c(-c4nc(-c5ccccc5-c5ccc(-c6ccccc6)cc5)cc(-c5cccc6c5sc5ccccc56)n4)cccc32)c2ccccc21]

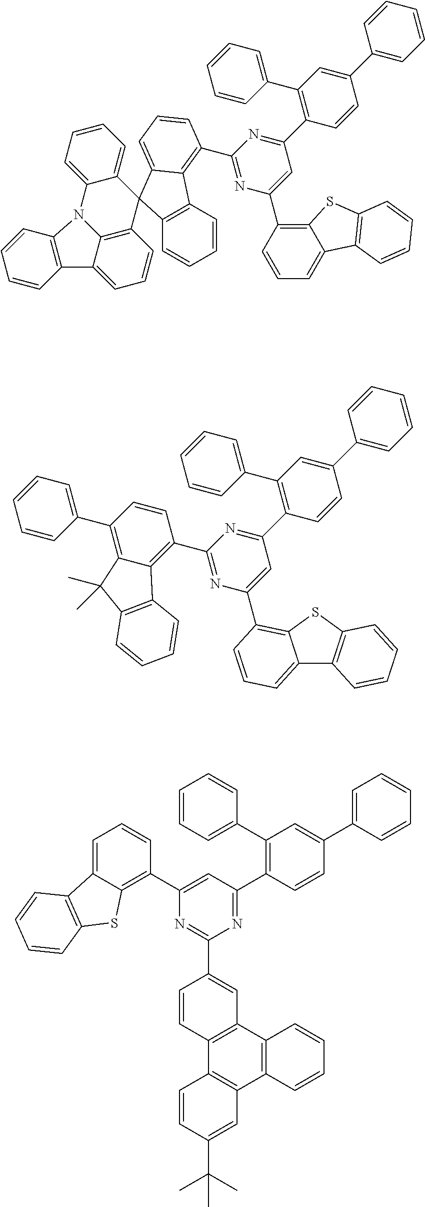 [SMILES: c1ccc(-c2ccc(-c3cc(-c4cccc5c4sc4ccccc45)nc(-c4cccc5c4-c4ccccc4C54c5ccccc5-n5c6ccccc6c6cccc4c65)n3)c(-c3ccccc3)c2)cc1 ; CC1(C)c2ccccc2-c2c(-c3nc(-c4ccc(-c5ccccc5)cc4-c4ccccc4)cc(-c4cccc5c4sc4ccccc45)n3)ccc(-c3ccccc3)c21 ; CC(C)(C)c1ccc2c3ccc(-c4nc(-c5ccc(-c6ccccc6)cc5-c5ccccc5)cc(-c5cccc6c5sc5ccccc56)n4)cc3c3ccccc3c2c1]

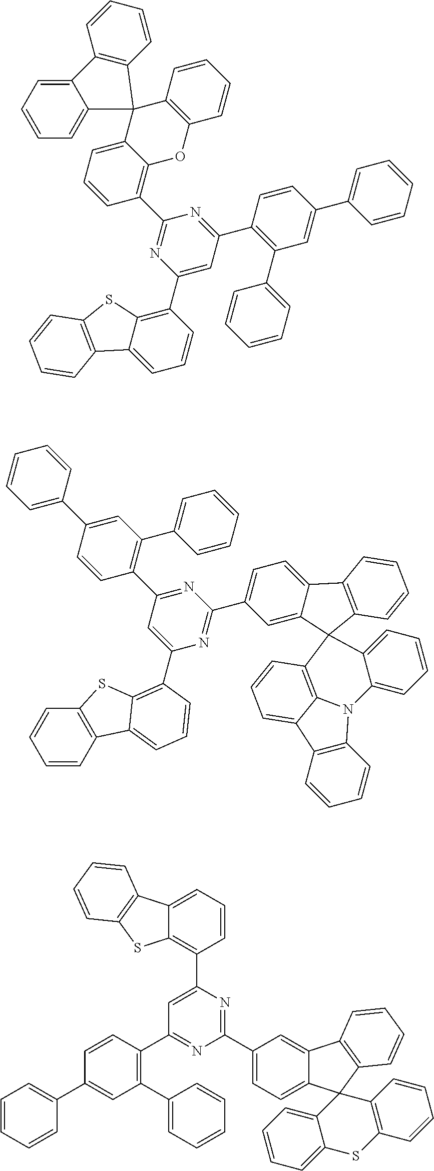 [SMILES: c1ccc(-c2ccc(-c3cc(-c4cccc5c4sc4ccccc45)nc(-c4cccc5c4Oc4ccccc4C54c5ccccc5-c5ccccc54)n3)c(-c3ccccc3)c2)cc1 ; c1ccc(-c2ccc(-c3cc(-c4cccc5c4sc4ccccc45)nc(-c4ccc5c(c4)C4(c6ccccc6-5)c5ccccc5-n5c6ccccc6c6cccc4c65)n3)c(-c3ccccc3)c2)cc1 ; c1ccc(-c2ccc(-c3cc(-c4cccc5c4sc4ccccc45)nc(-c4ccc5c(c4)-c4ccccc4C54c5ccccc5Sc5ccccc54)n3)c(-c3ccccc3)c2)cc1]

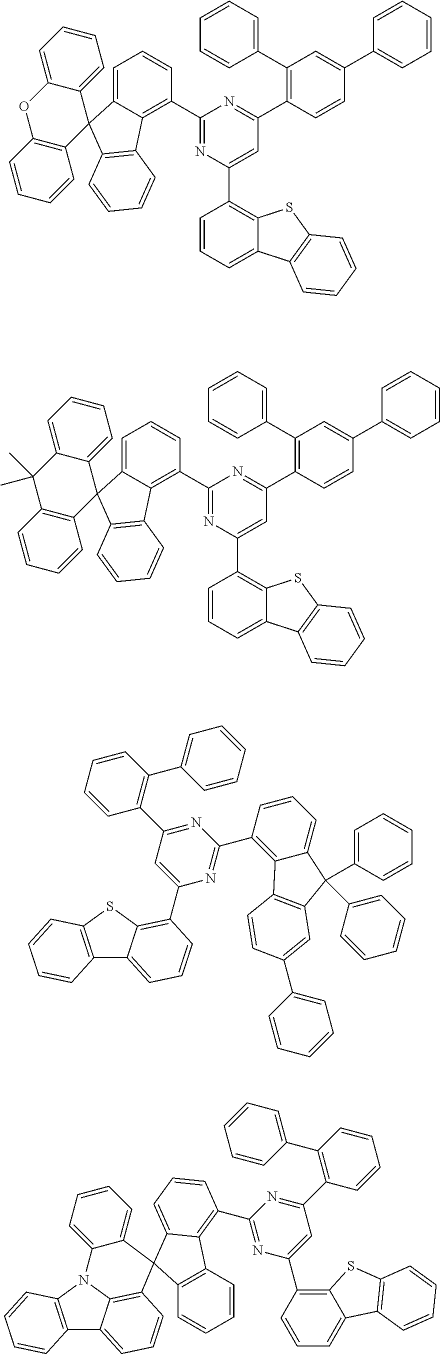 [SMILES: c1ccc(-c2ccc(-c3cc(-c4cccc5c4sc4ccccc45)nc(-c4cccc5c4-c4ccccc4C54c5ccccc5Oc5ccccc54)n3)c(-c3ccccc3)c2)cc1 ; CC1(C)c2ccccc2C2(c3ccccc3-c3c(-c4nc(-c5ccc(-c6ccccc6)cc5-c5ccccc5)cc(-c5cccc6c5sc5ccccc56)n4)cccc32)c2ccccc21 ; c1ccc(-c2ccc3c(c2)C(c2ccccc2)(c2ccccc2)c2cccc(-c4nc(-c5ccccc5-c5ccccc5)cc(-c5cccc6c5sc5ccccc56)n4)c2-3)cc1 ; c1ccc(-c2ccccc2-c2cc(-c3cccc4c3sc3ccccc34)nc(-c3cccc4c3-c3ccccc3C43c4ccccc4-n4c5ccccc5c5cccc3c54)n2)cc1]

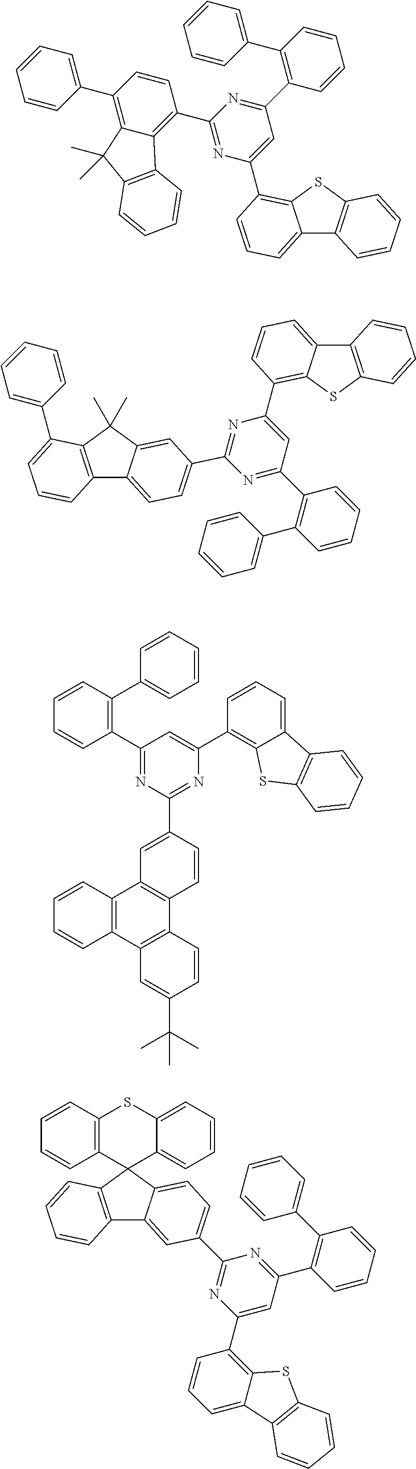

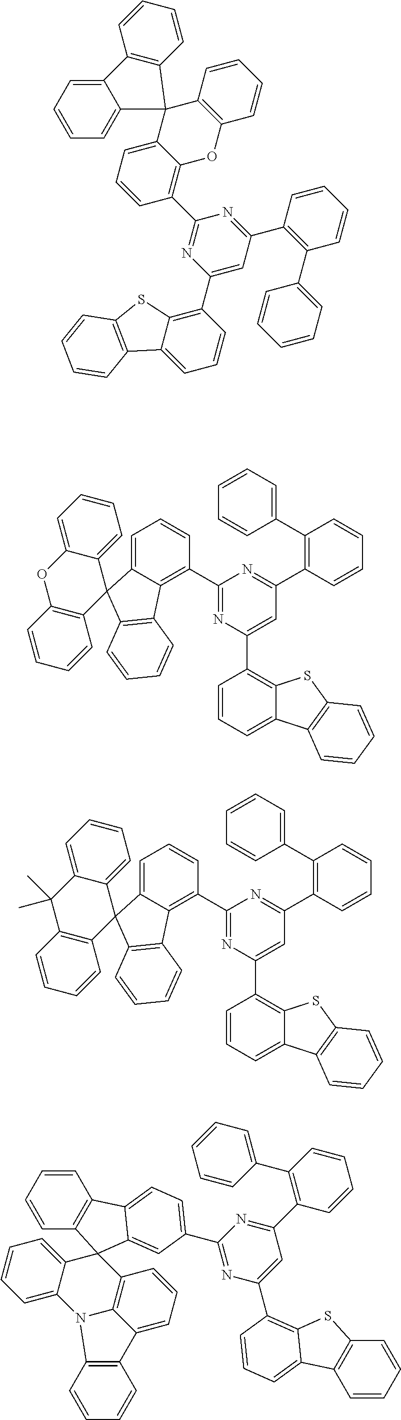 [SMILES: c1ccc(-c2ccccc2-c2cc(-c3cccc4c3sc3ccccc34)nc(-c3cccc4c3Oc3ccccc3C43c4ccccc4-c4ccccc43)n2)cc1 ; c1ccc(-c2ccccc2-c2cc(-c3cccc4c3sc3ccccc34)nc(-c3cccc4c3-c3ccccc3C43c4ccccc4Oc4ccccc43)n2)cc1 ; CC1(C)c2ccccc2C2(c3ccccc3-c3c(-c4nc(-c5ccccc5-c5ccccc5)cc(-c5cccc6c5sc5ccccc56)n4)cccc32)c2ccccc21 ; c1ccc(-c2ccccc2-c2cc(-c3cccc4c3sc3ccccc34)nc(-c3ccc4c(c3)C3(c5ccccc5-4)c4ccccc4-n4c5ccccc5c5cccc3c54)n2)cc1]

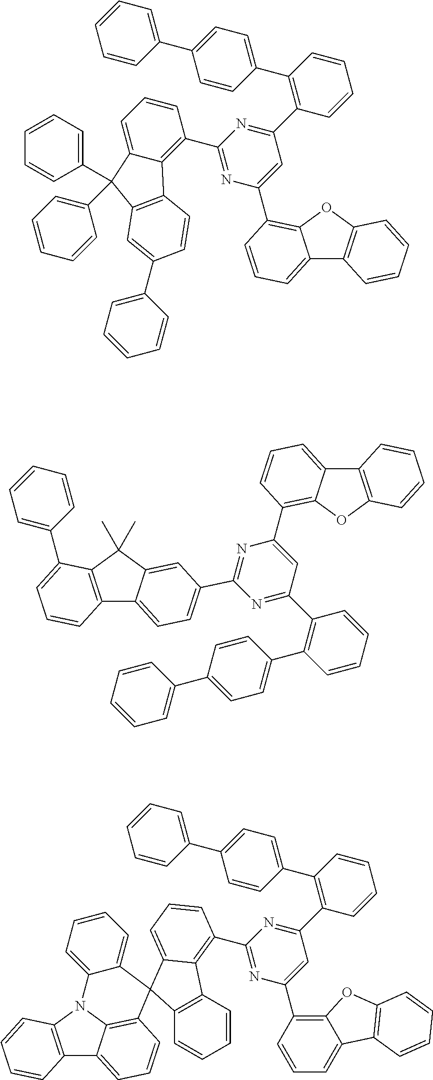 [SMILES: c1ccc(-c2ccc(-c3ccccc3-c3cc(-c4cccc5c4oc4ccccc45)nc(-c4cccc5c4-c4ccc(-c6ccccc6)cc4C5(c4ccccc4)c4ccccc4)n3)cc2)cc1 ; CC1(C)c2cc(-c3nc(-c4ccccc4-c4ccc(-c5ccccc5)cc4)cc(-c4cccc5c4oc4ccccc45)n3)ccc2-c2cccc(-c3ccccc3)c21 ; c1ccc(-c2ccc(-c3ccccc3-c3cc(-c4cccc5c4oc4ccccc45)nc(-c4cccc5c4-c4ccccc4C54c5ccccc5-n5c6ccccc6c6cccc4c65)n3)cc2)cc1]

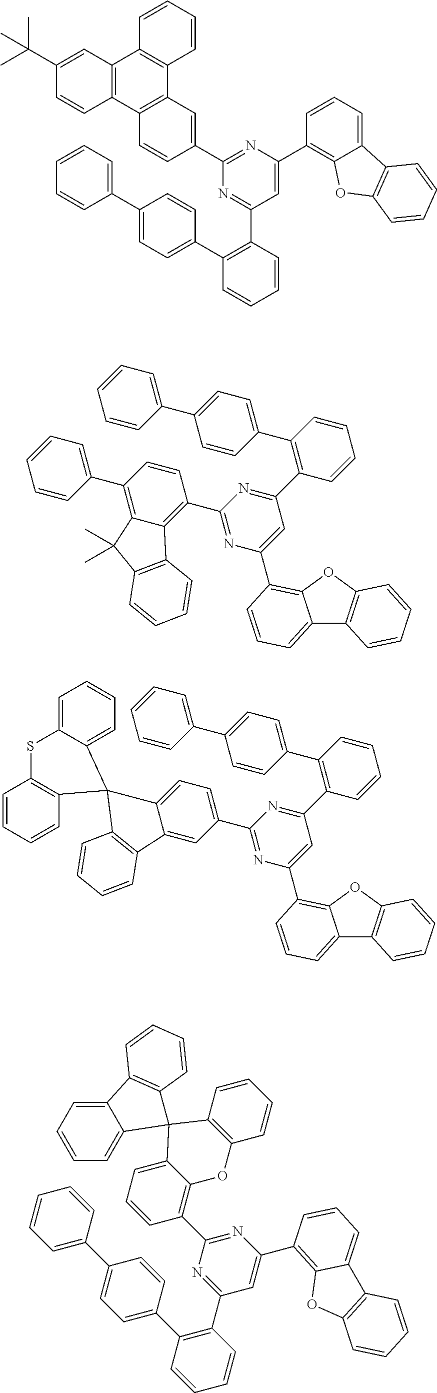 [SMILES: CC(C)(C)c1ccc2c3ccc(-c4nc(-c5ccccc5-c5ccc(-c6ccccc6)cc5)cc(-c5cccc6c5oc5ccccc56)n4)cc3c3ccccc3c2c1 ; CC1(C)c2ccccc2-c2c(-c3nc(-c4ccccc4-c4ccc(-c5ccccc5)cc4)cc(-c4cccc5c4oc4ccccc45)n3)ccc(-c3ccccc3)c21 ; c1ccc(-c2ccc(-c3ccccc3-c3cc(-c4cccc5c4oc4ccccc45)nc(-c4ccc5c(c4)-c4ccccc4C54c5ccccc5Sc5ccccc54)n3)cc2)cc1 ; c1ccc(-c2ccc(-c3ccccc3-c3cc(-c4cccc5c4oc4ccccc45)nc(-c4cccc5c4Oc4ccccc4C54c5ccccc5-c5ccccc54)n3)cc2)cc1]

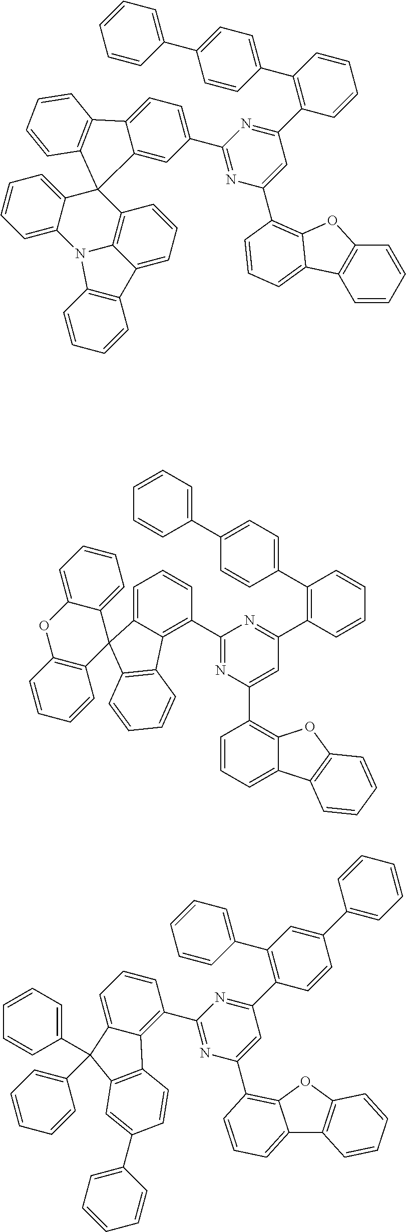

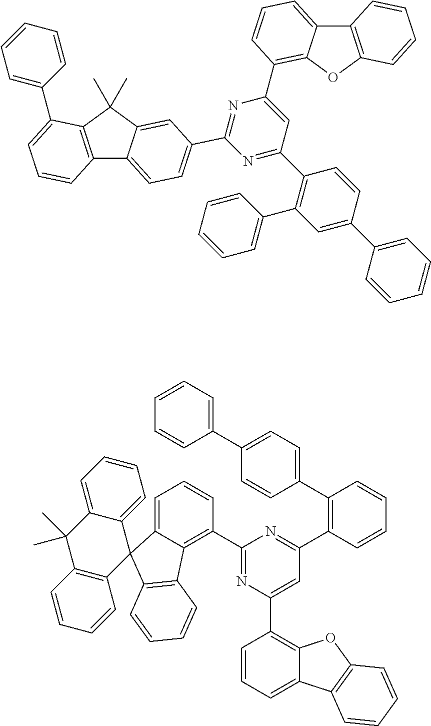 [SMILES: CC1(C)c2cc(-c3nc(-c4ccc(-c5ccccc5)cc4-c4ccccc4)cc(-c4cccc5c4oc4ccccc45)n3)ccc2-c2cccc(-c3ccccc3)c21 ; CC1(C)c2ccccc2C2(c3ccccc3-c3c(-c4nc(-c5ccccc5-c5ccc(-c6ccccc6)cc5)cc(-c5cccc6c5oc5ccccc56)n4)cccc32)c2ccccc21]

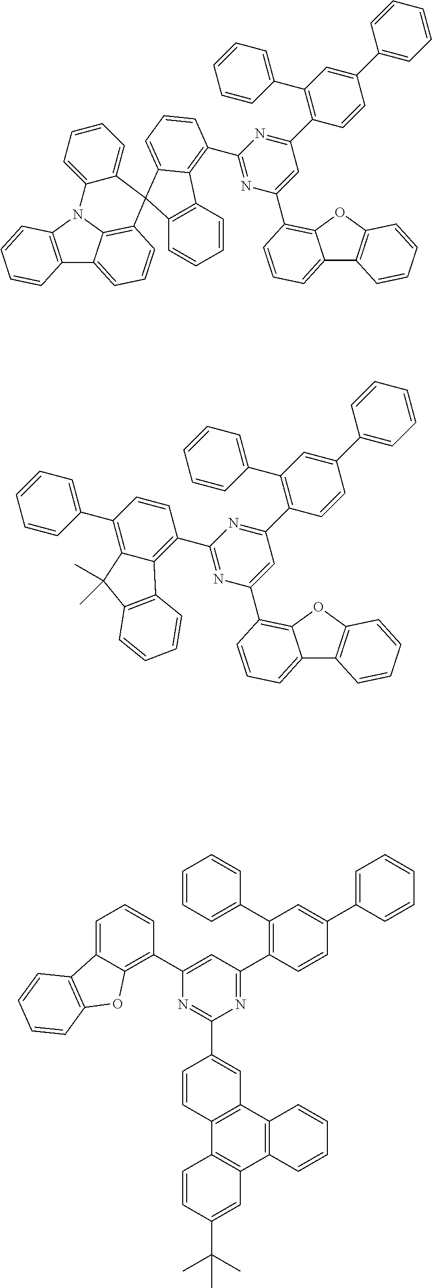 [SMILES: c1ccc(-c2ccc(-c3cc(-c4cccc5c4oc4ccccc45)nc(-c4cccc5c4-c4ccccc4C54c5ccccc5-n5c6ccccc6c6cccc4c65)n3)c(-c3ccccc3)c2)cc1 ; CC1(C)c2ccccc2-c2c(-c3nc(-c4ccc(-c5ccccc5)cc4-c4ccccc4)cc(-c4cccc5c4oc4ccccc45)n3)ccc(-c3ccccc3)c21 ; CC(C)(C)c1ccc2c3ccc(-c4nc(-c5ccc(-c6ccccc6)cc5-c5ccccc5)cc(-c5cccc6c5oc5ccccc56)n4)cc3c3ccccc3c2c1]

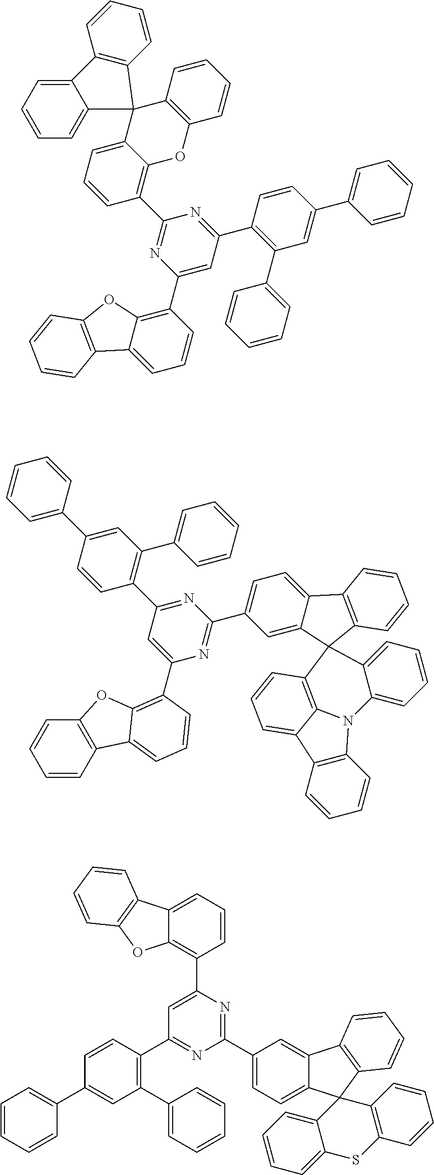 [SMILES: c1ccc(-c2ccc(-c3cc(-c4cccc5c4oc4ccccc45)nc(-c4cccc5c4Oc4ccccc4C54c5ccccc5-c5ccccc54)n3)c(-c3ccccc3)c2)cc1 ; c1ccc(-c2ccc(-c3cc(-c4cccc5c4oc4ccccc45)nc(-c4ccc5c(c4)C4(c6ccccc6-5)c5ccccc5-n5c6ccccc6c6cccc4c65)n3)c(-c3ccccc3)c2)cc1 ; c1ccc(-c2ccc(-c3cc(-c4cccc5c4oc4ccccc45)nc(-c4ccc5c(c4)-c4ccccc4C54c5ccccc5Sc5ccccc54)n3)c(-c3ccccc3)c2)cc1]

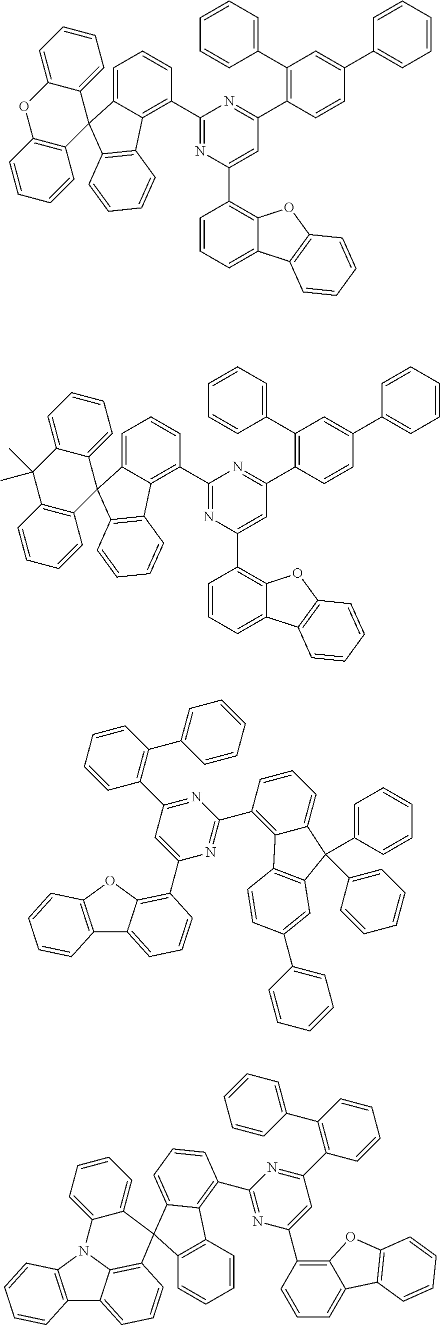 [SMILES: c1ccc(-c2ccc(-c3cc(-c4cccc5c4oc4ccccc45)nc(-c4cccc5c4-c4ccccc4C54c5ccccc5Oc5ccccc54)n3)c(-c3ccccc3)c2)cc1 ; CC1(C)c2ccccc2C2(c3ccccc3-c3c(-c4nc(-c5ccc(-c6ccccc6)cc5-c5ccccc5)cc(-c5cccc6c5oc5ccccc56)n4)cccc32)c2ccccc21 ; c1ccc(-c2ccc3c(c2)C(c2ccccc2)(c2ccccc2)c2cccc(-c4nc(-c5ccccc5-c5ccccc5)cc(-c5cccc6c5oc5ccccc56)n4)c2-3)cc1 ; c1ccc(-c2ccccc2-c2cc(-c3cccc4c3oc3ccccc34)nc(-c3cccc4c3-c3ccccc3C43c4ccccc4-n4c5ccccc5c5cccc3c54)n2)cc1]

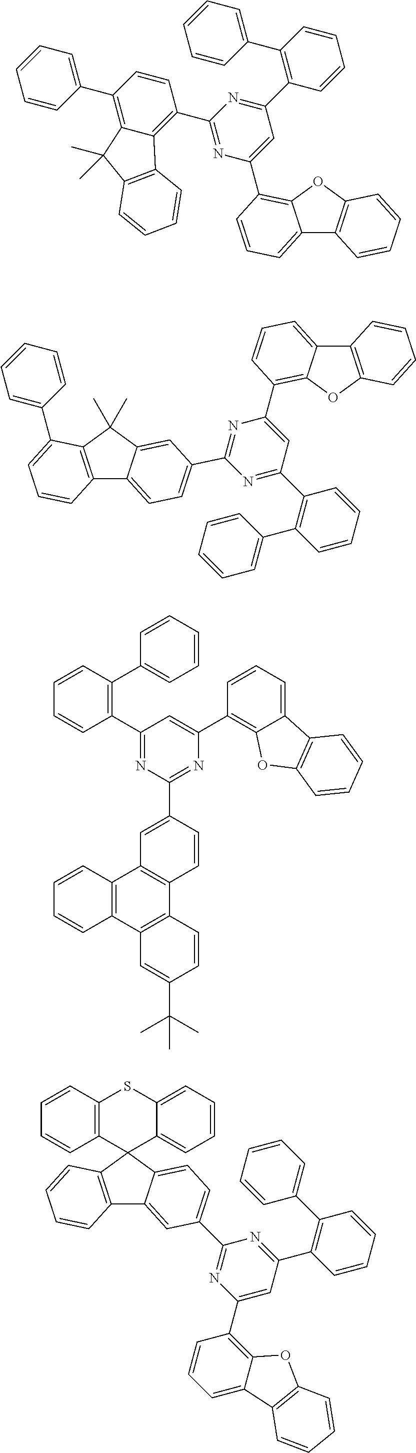

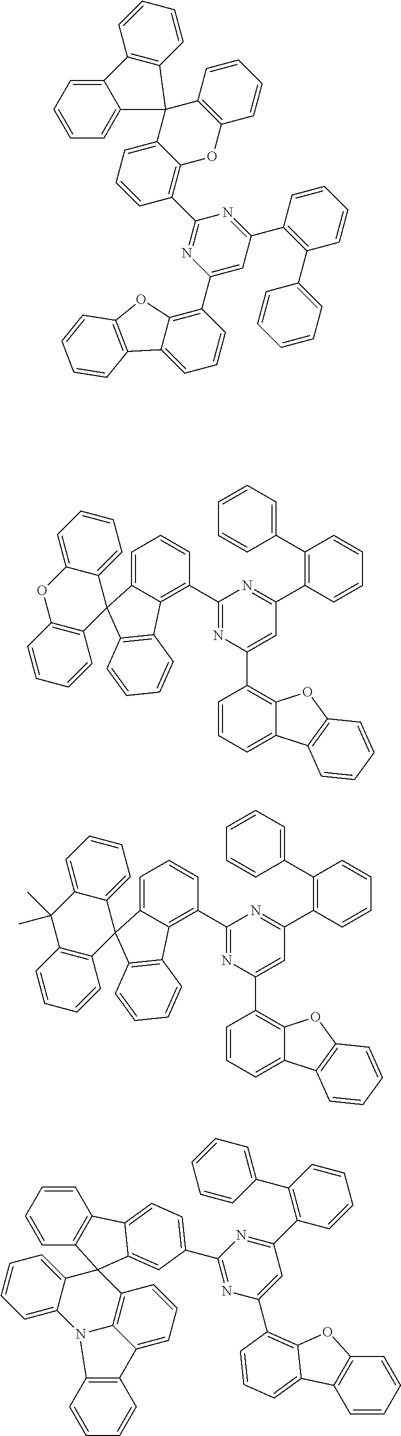 [SMILES: c1ccc(-c2ccccc2-c2cc(-c3cccc4c3oc3ccccc34)nc(-c3cccc4c3Oc3ccccc3C43c4ccccc4-c4ccccc43)n2)cc1 ; c1ccc(-c2ccccc2-c2cc(-c3cccc4c3oc3ccccc34)nc(-c3cccc4c3-c3ccccc3C43c4ccccc4Oc4ccccc43)n2)cc1 ; CC1(C)c2ccccc2C2(c3ccccc3-c3c(-c4nc(-c5ccccc5-c5ccccc5)cc(-c5cccc6c5oc5ccccc56)n4)cccc32)c2ccccc21 ; c1ccc(-c2ccccc2-c2cc(-c3cccc4c3oc3ccccc34)nc(-c3ccc4c(c3)C3(c5ccccc5-4)c4ccccc4-n4c5ccccc5c5cccc3c54)n2)cc1]

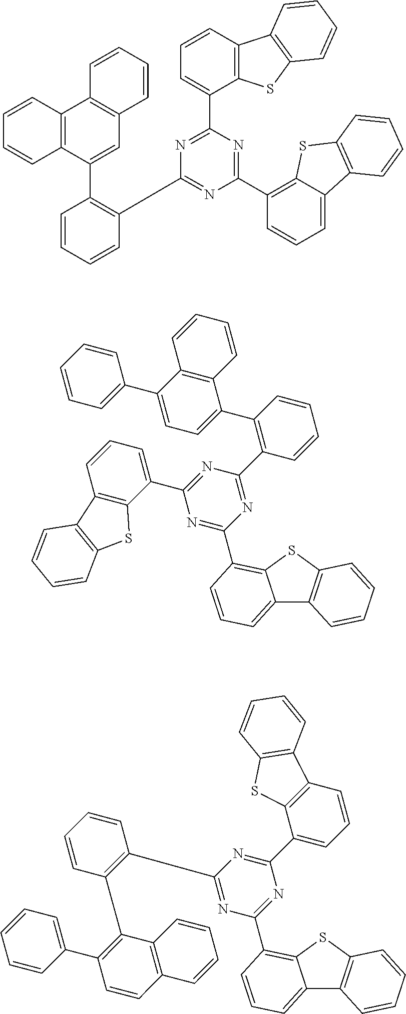 [SMILES: c1ccc(-c2cc3ccccc3c3ccccc23)c(-c2nc(-c3cccc4c3sc3ccccc34)nc(-c3cccc4c3sc3ccccc34)n2)c1 ; c1ccc(-c2ccc(-c3ccccc3-c3nc(-c4cccc5c4sc4ccccc45)nc(-c4cccc5c4sc4ccccc45)n3)c3ccccc23)cc1 ; c1ccc(-c2ccc3ccccc3c2-c2ccccc2-c2nc(-c3cccc4c3sc3ccccc34)nc(-c3cccc4c3sc3ccccc34)n2)cc1]

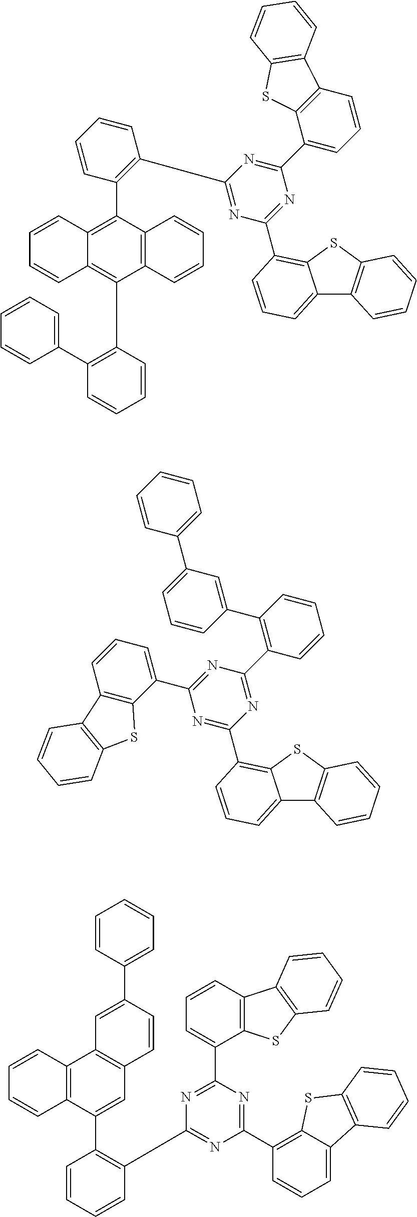 [SMILES: c1ccc(-c2ccccc2-c2c3ccccc3c(-c3ccccc3-c3nc(-c4cccc5c4sc4ccccc45)nc(-c4cccc5c4sc4ccccc45)n3)c3ccccc23)cc1 ; c1ccc(-c2cccc(-c3ccccc3-c3nc(-c4cccc5c4sc4ccccc45)nc(-c4cccc5c4sc4ccccc45)n3)c2)cc1 ; c1ccc(-c2ccc3cc(-c4ccccc4-c4nc(-c5cccc6c5sc5ccccc56)nc(-c5cccc6c5sc5ccccc56)n4)c4ccccc4c3c2)cc1]

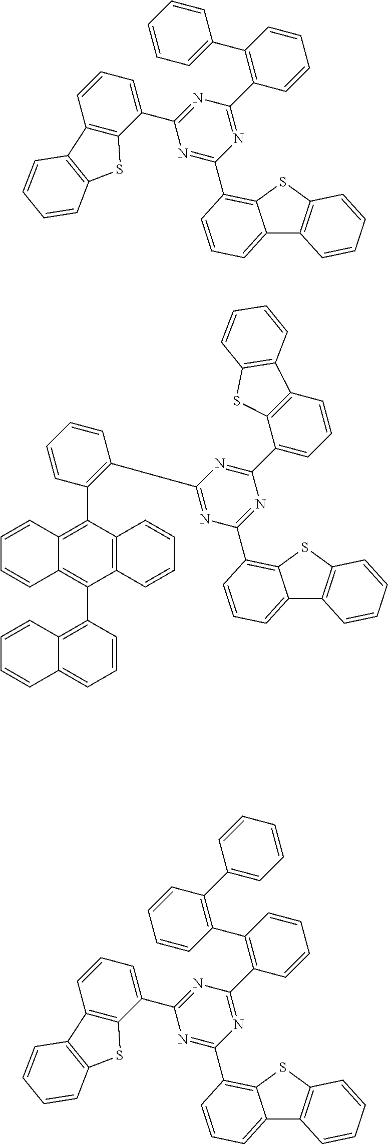 [SMILES: c1ccc(-c2ccccc2-c2nc(-c3cccc4c3sc3ccccc34)nc(-c3cccc4c3sc3ccccc34)n2)cc1 ; c1ccc(-c2c3ccccc3c(-c3cccc4ccccc34)c3ccccc23)c(-c2nc(-c3cccc4c3sc3ccccc34)nc(-c3cccc4c3sc3ccccc34)n2)c1 ; c1ccc(-c2ccccc2-c2ccccc2-c2nc(-c3cccc4c3sc3ccccc34)nc(-c3cccc4c3sc3ccccc34)n2)cc1]

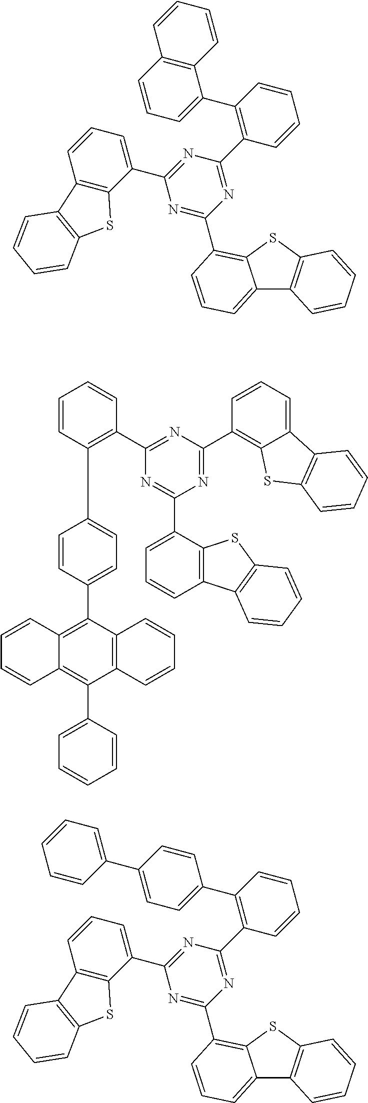

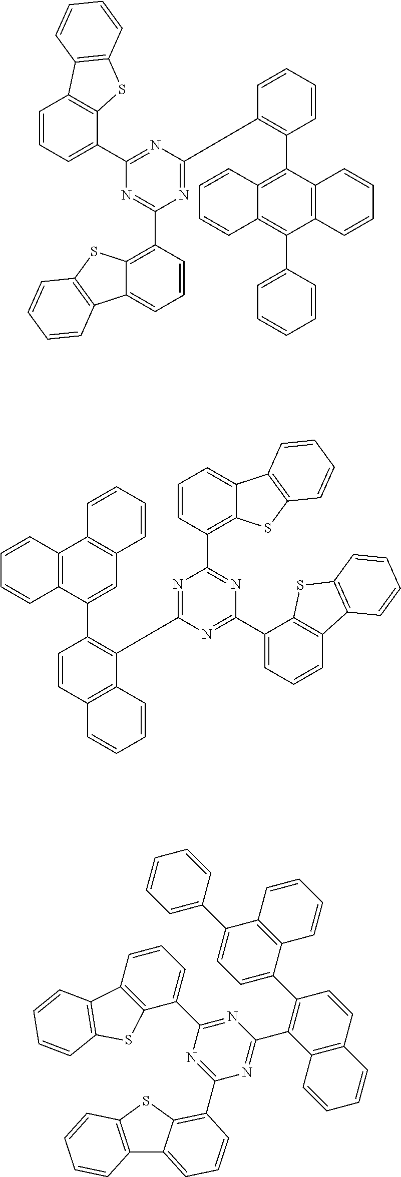 [SMILES: c1ccc(-c2c3ccccc3c(-c3ccccc3-c3nc(-c4cccc5c4sc4ccccc45)nc(-c4cccc5c4sc4ccccc45)n3)c3ccccc23)cc1 ; c1ccc2c(-c3nc(-c4cccc5c4sc4ccccc45)nc(-c4cccc5c4sc4ccccc45)n3)c(-c3cc4ccccc4c4ccccc34)ccc2c1 ; c1ccc(-c2ccc(-c3ccc4ccccc4c3-c3nc(-c4cccc5c4sc4ccccc45)nc(-c4cccc5c4sc4ccccc45)n3)c3ccccc23)cc1]

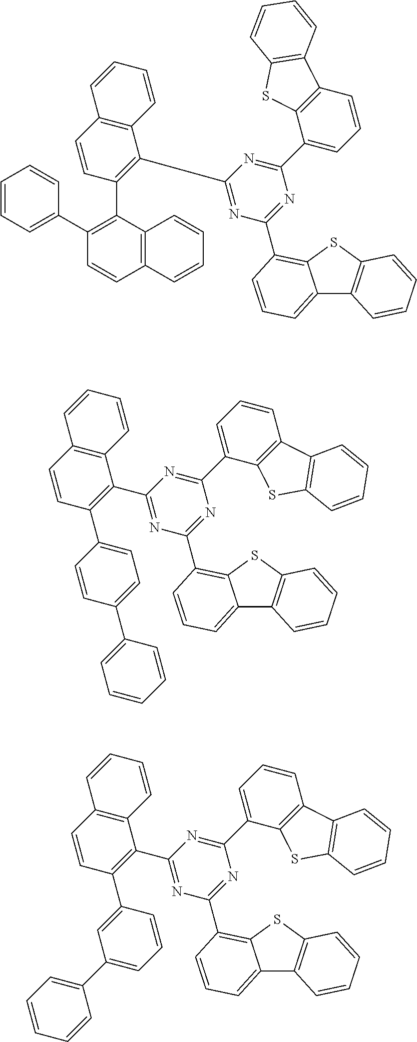 [SMILES: c1ccc(-c2ccc3ccccc3c2-c2ccc3ccccc3c2-c2nc(-c3cccc4c3sc3ccccc34)nc(-c3cccc4c3sc3ccccc34)n2)cc1 ; c1ccc(-c2ccc(-c3ccc4ccccc4c3-c3nc(-c4cccc5c4sc4ccccc45)nc(-c4cccc5c4sc4ccccc45)n3)cc2)cc1 ; c1ccc(-c2cccc(-c3ccc4ccccc4c3-c3nc(-c4cccc5c4sc4ccccc45)nc(-c4cccc5c4sc4ccccc45)n3)c2)cc1]

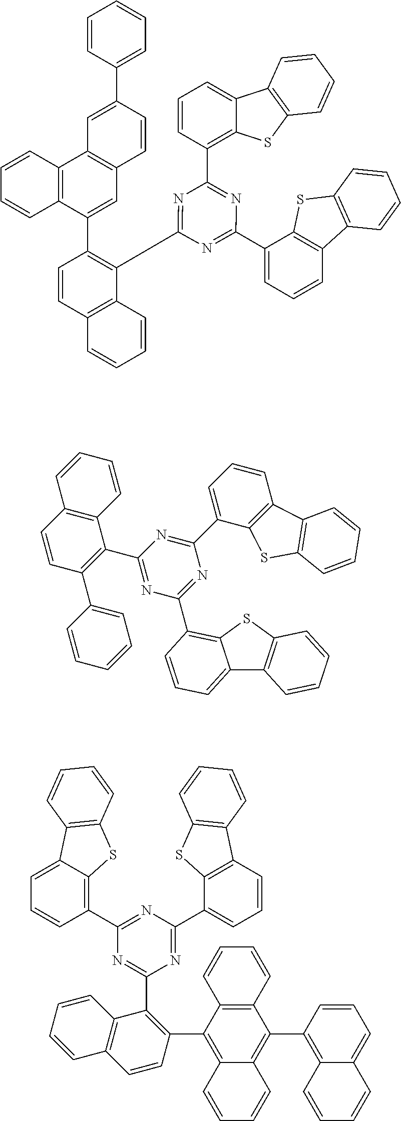 [SMILES: c1ccc(-c2ccc3cc(-c4ccc5ccccc5c4-c4nc(-c5cccc6c5sc5ccccc56)nc(-c5cccc6c5sc5ccccc56)n4)c4ccccc4c3c2)cc1 ; c1ccc(-c2ccc3ccccc3c2-c2nc(-c3cccc4c3sc3ccccc34)nc(-c3cccc4c3sc3ccccc34)n2)cc1 ; c1ccc2c(-c3c4ccccc4c(-c4ccc5ccccc5c4-c4nc(-c5cccc6c5sc5ccccc56)nc(-c5cccc6c5sc5ccccc56)n4)c4ccccc34)cccc2c1]

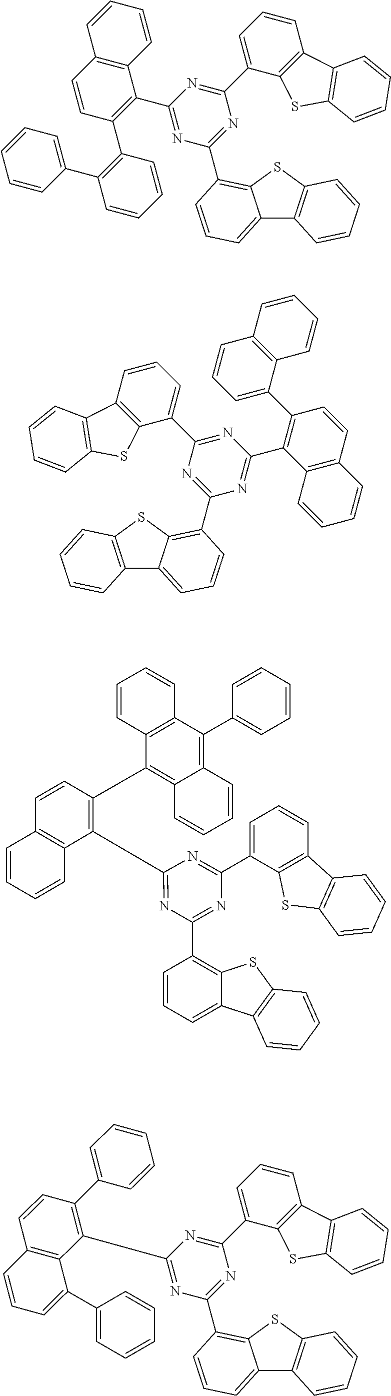 [SMILES: c1ccc(-c2ccccc2-c2ccc3ccccc3c2-c2nc(-c3cccc4c3sc3ccccc34)nc(-c3cccc4c3sc3ccccc34)n2)cc1 ; c1ccc2c(-c3ccc4ccccc4c3-c3nc(-c4cccc5c4sc4ccccc45)nc(-c4cccc5c4sc4ccccc45)n3)cccc2c1 ; c1ccc(-c2c3ccccc3c(-c3ccc4ccccc4c3-c3nc(-c4cccc5c4sc4ccccc45)nc(-c4cccc5c4sc4ccccc45)n3)c3ccccc23)cc1 ; c1ccc(-c2ccc3cccc(-c4ccccc4)c3c2-c2nc(-c3cccc4c3sc3ccccc34)nc(-c3cccc4c3sc3ccccc34)n2)cc1]

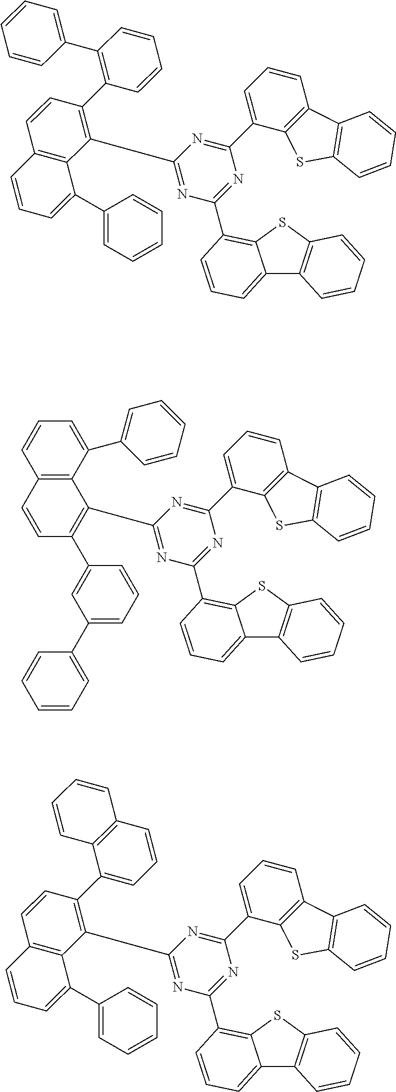 [SMILES: c1ccc(-c2ccccc2-c2ccc3cccc(-c4ccccc4)c3c2-c2nc(-c3cccc4c3sc3ccccc34)nc(-c3cccc4c3sc3ccccc34)n2)cc1 ; c1ccc(-c2cccc(-c3ccc4cccc(-c5ccccc5)c4c3-c3nc(-c4cccc5c4sc4ccccc45)nc(-c4cccc5c4sc4ccccc45)n3)c2)cc1 ; c1ccc(-c2cccc3ccc(-c4cccc5ccccc45)c(-c4nc(-c5cccc6c5sc5ccccc56)nc(-c5cccc6c5sc5ccccc56)n4)c23)cc1]

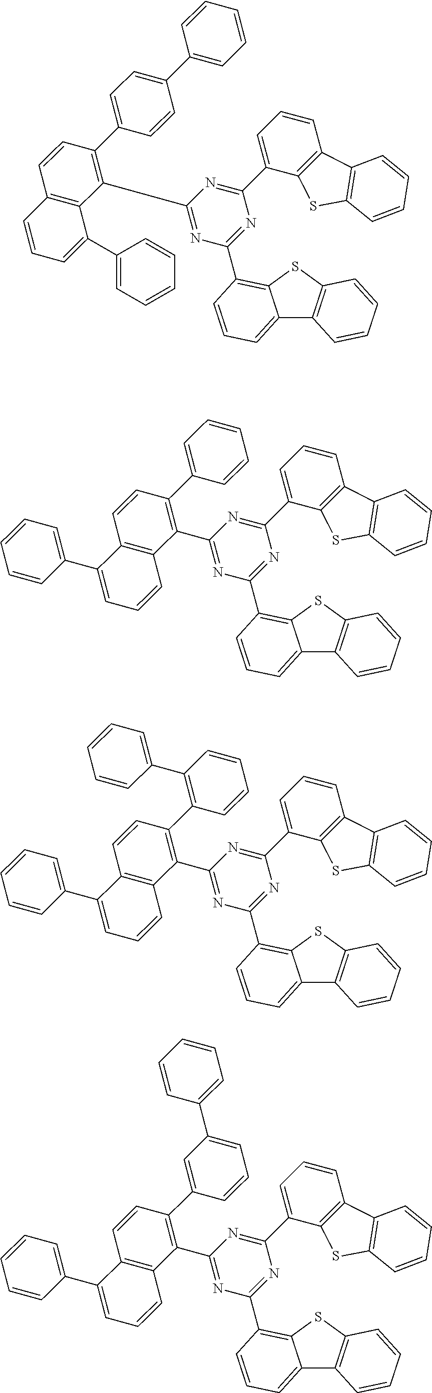 [SMILES: c1ccc(-c2ccc(-c3ccc4cccc(-c5ccccc5)c4c3-c3nc(-c4cccc5c4sc4ccccc45)nc(-c4cccc5c4sc4ccccc45)n3)cc2)cc1 ; c1ccc(-c2ccc3c(-c4ccccc4)cccc3c2-c2nc(-c3cccc4c3sc3ccccc34)nc(-c3cccc4c3sc3ccccc34)n2)cc1 ; c1ccc(-c2ccccc2-c2ccc3c(-c4ccccc4)cccc3c2-c2nc(-c3cccc4c3sc3ccccc34)nc(-c3cccc4c3sc3ccccc34)n2)cc1 ; c1ccc(-c2cccc(-c3ccc4c(-c5ccccc5)cccc4c3-c3nc(-c4cccc5c4sc4ccccc45)nc(-c4cccc5c4sc4ccccc45)n3)c2)cc1]

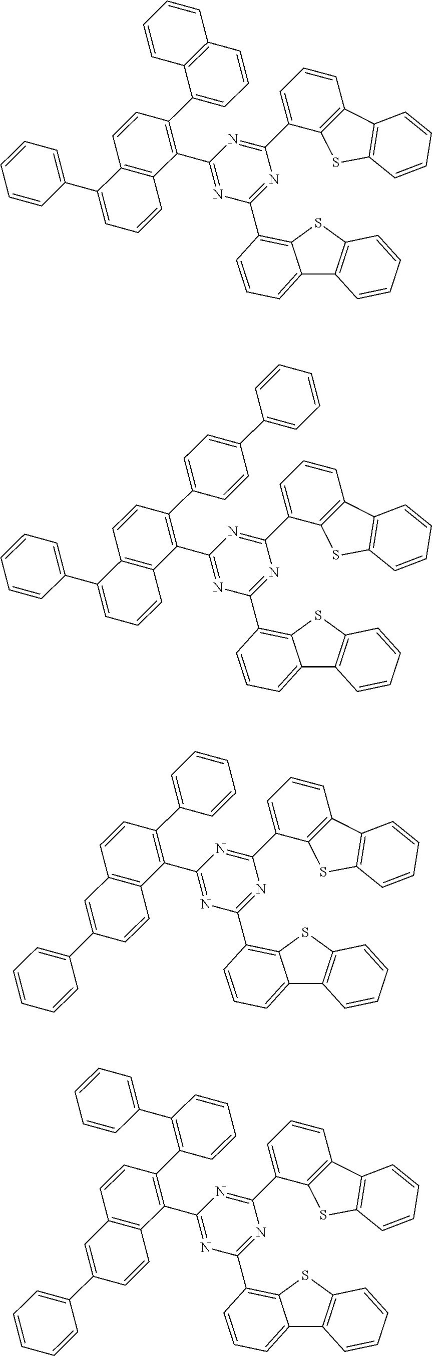 [SMILES: c1ccc(-c2cccc3c(-c4nc(-c5cccc6c5sc5ccccc56)nc(-c5cccc6c5sc5ccccc56)n4)c(-c4cccc5ccccc45)ccc23)cc1 ; c1ccc(-c2ccc(-c3ccc4c(-c5ccccc5)cccc4c3-c3nc(-c4cccc5c4sc4ccccc45)nc(-c4cccc5c4sc4ccccc45)n3)cc2)cc1 ; c1ccc(-c2ccc3c(-c4nc(-c5cccc6c5sc5ccccc56)nc(-c5cccc6c5sc5ccccc56)n4)c(-c4ccccc4)ccc3c2)cc1 ; c1ccc(-c2ccc3c(-c4nc(-c5cccc6c5sc5ccccc56)nc(-c5cccc6c5sc5ccccc56)n4)c(-c4ccccc4-c4ccccc4)ccc3c2)cc1]

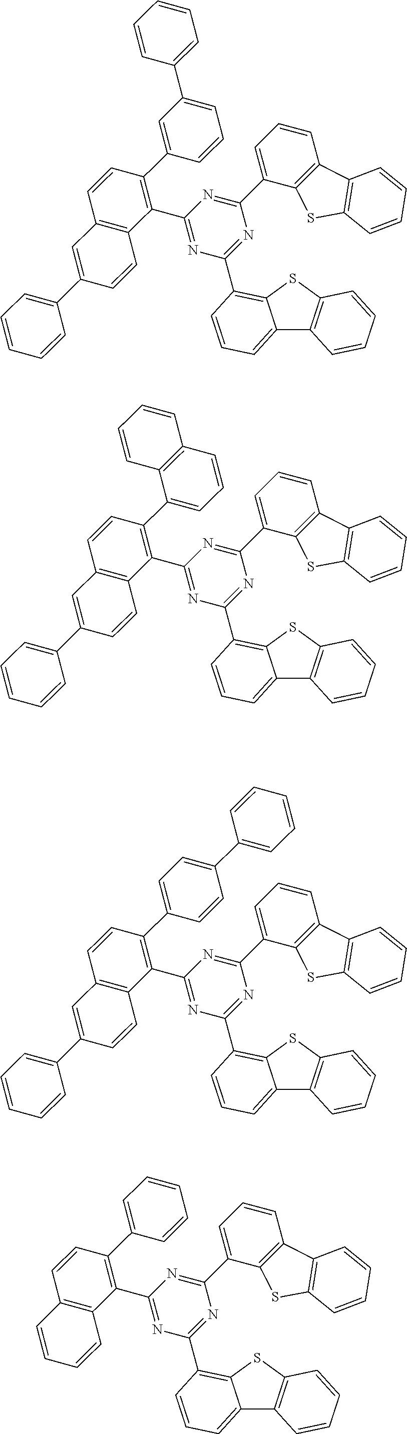 [SMILES: c1ccc(-c2cccc(-c3ccc4cc(-c5ccccc5)ccc4c3-c3nc(-c4cccc5c4sc4ccccc45)nc(-c4cccc5c4sc4ccccc45)n3)c2)cc1 ; c1ccc(-c2ccc3c(-c4nc(-c5cccc6c5sc5ccccc56)nc(-c5cccc6c5sc5ccccc56)n4)c(-c4cccc5ccccc45)ccc3c2)cc1 ; c1ccc(-c2ccc(-c3ccc4cc(-c5ccccc5)ccc4c3-c3nc(-c4cccc5c4sc4ccccc45)nc(-c4cccc5c4sc4ccccc45)n3)cc2)cc1 ; c1ccc(-c2ccc3ccccc3c2-c2nc(-c3cccc4c3sc3ccccc34)nc(-c3cccc4c3sc3ccccc34)n2)cc1]

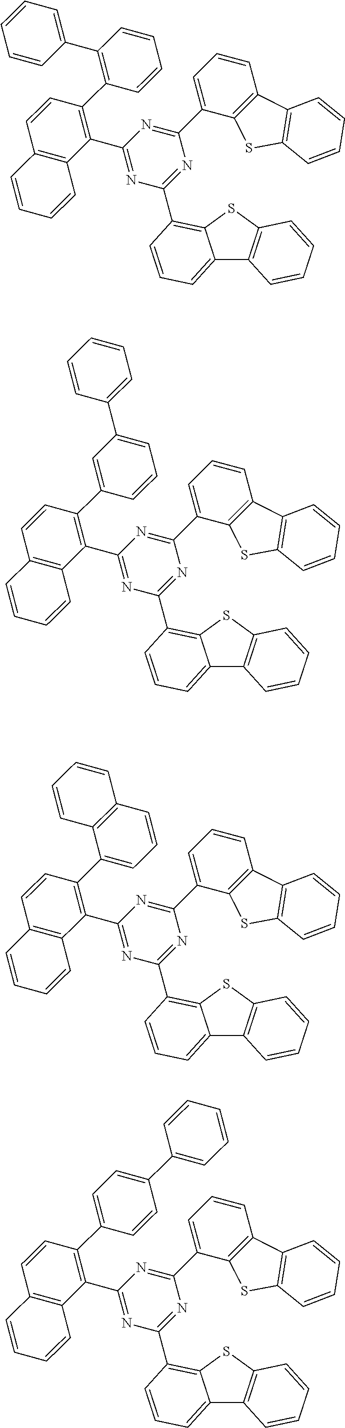

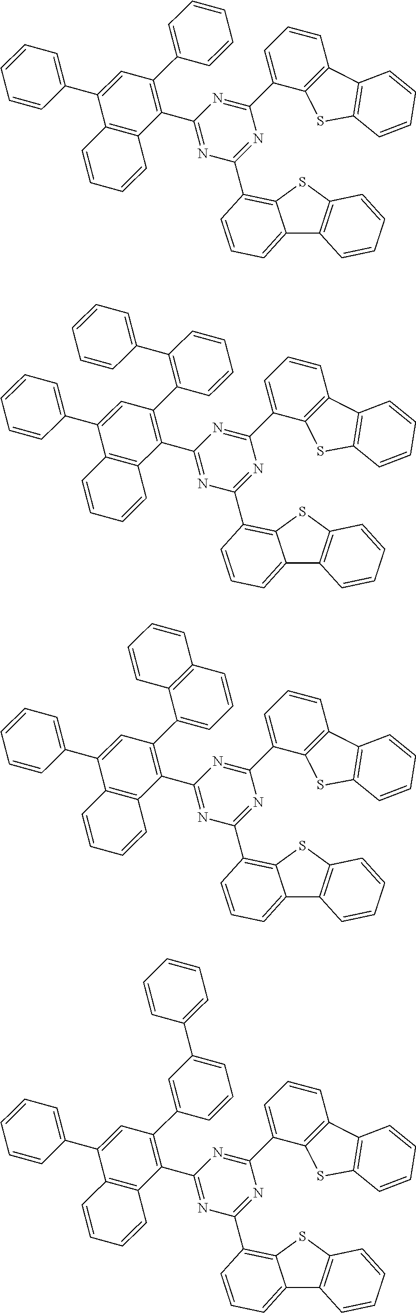 [SMILES: c1ccc(-c2cc(-c3ccccc3)c3ccccc3c2-c2nc(-c3cccc4c3sc3ccccc34)nc(-c3cccc4c3sc3ccccc34)n2)cc1 ; c1ccc(-c2ccccc2-c2cc(-c3ccccc3)c3ccccc3c2-c2nc(-c3cccc4c3sc3ccccc34)nc(-c3cccc4c3sc3ccccc34)n2)cc1 ; c1ccc(-c2cc(-c3cccc4ccccc34)c(-c3nc(-c4cccc5c4sc4ccccc45)nc(-c4cccc5c4sc4ccccc45)n3)c3ccccc23)cc1 ; c1ccc(-c2cccc(-c3cc(-c4ccccc4)c4ccccc4c3-c3nc(-c4cccc5c4sc4ccccc45)nc(-c4cccc5c4sc4ccccc45)n3)c2)cc1]

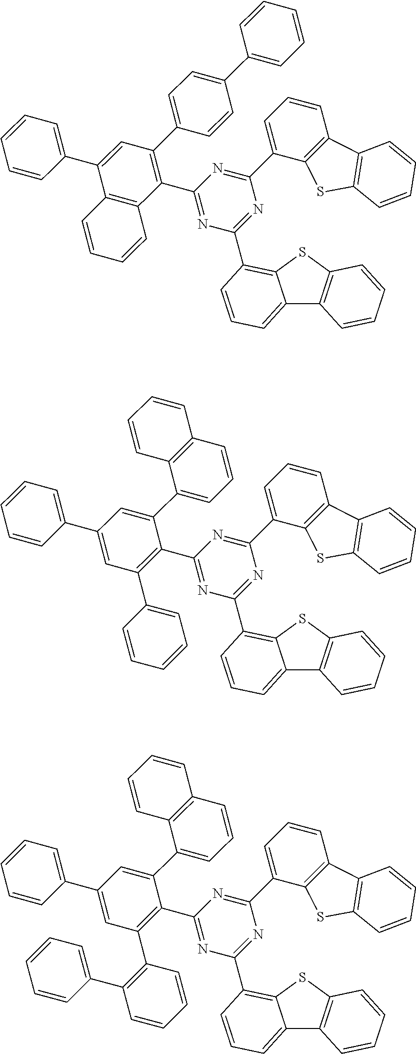 [SMILES: c1ccc(-c2ccc(-c3cc(-c4ccccc4)c4ccccc4c3-c3nc(-c4cccc5c4sc4ccccc45)nc(-c4cccc5c4sc4ccccc45)n3)cc2)cc1 ; c1ccc(-c2cc(-c3ccccc3)c(-c3nc(-c4cccc5c4sc4ccccc45)nc(-c4cccc5c4sc4ccccc45)n3)c(-c3cccc4ccccc34)c2)cc1 ; c1ccc(-c2cc(-c3ccccc3-c3ccccc3)c(-c3nc(-c4cccc5c4sc4ccccc45)nc(-c4cccc5c4sc4ccccc45)n3)c(-c3cccc4ccccc34)c2)cc1]

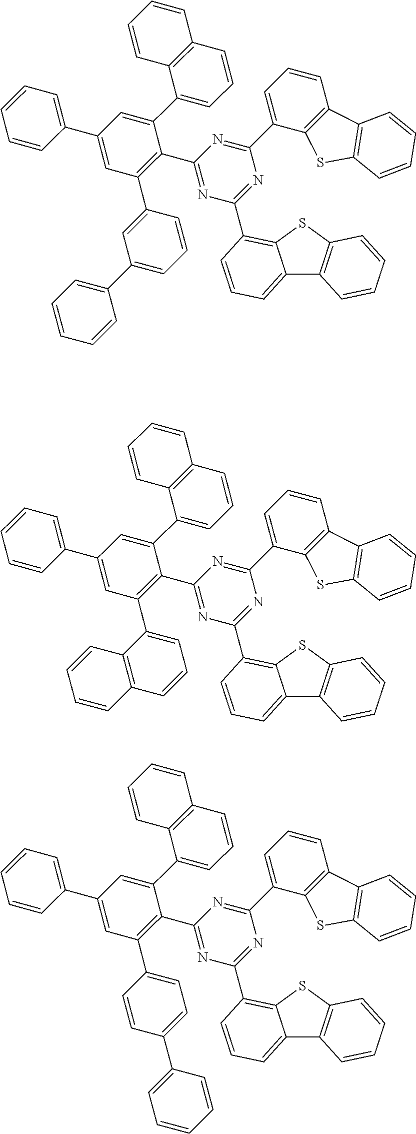 [SMILES: c1ccc(-c2cccc(-c3cc(-c4ccccc4)cc(-c4cccc5ccccc45)c3-c3nc(-c4cccc5c4sc4ccccc45)nc(-c4cccc5c4sc4ccccc45)n3)c2)cc1 ; c1ccc(-c2cc(-c3cccc4ccccc34)c(-c3nc(-c4cccc5c4sc4ccccc45)nc(-c4cccc5c4sc4ccccc45)n3)c(-c3cccc4ccccc34)c2)cc1 ; c1ccc(-c2ccc(-c3cc(-c4ccccc4)cc(-c4cccc5ccccc45)c3-c3nc(-c4cccc5c4sc4ccccc45)nc(-c4cccc5c4sc4ccccc45)n3)cc2)cc1]

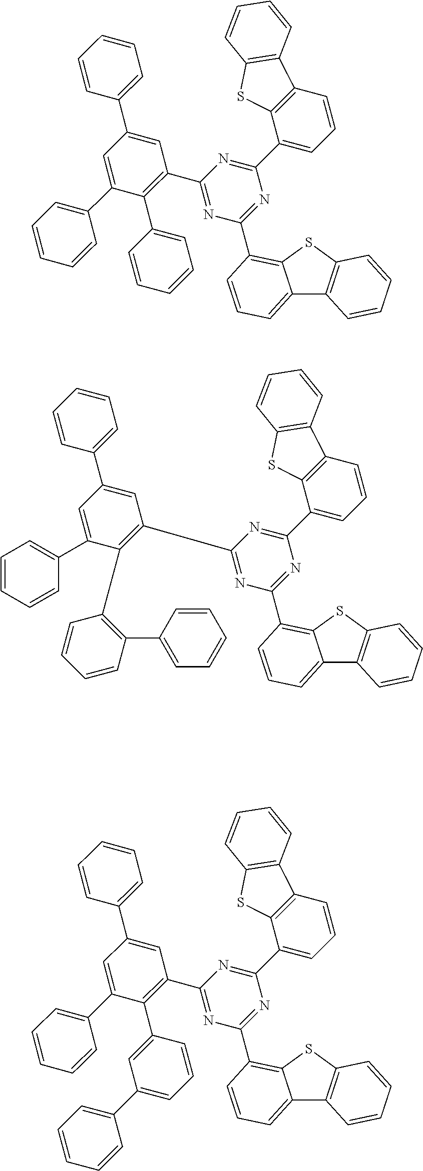 [SMILES: c1ccc(-c2cc(-c3ccccc3)c(-c3ccccc3)c(-c3nc(-c4cccc5c4sc4ccccc45)nc(-c4cccc5c4sc4ccccc45)n3)c2)cc1 ; c1ccc(-c2cc(-c3ccccc3)c(-c3ccccc3-c3ccccc3)c(-c3nc(-c4cccc5c4sc4ccccc45)nc(-c4cccc5c4sc4ccccc45)n3)c2)cc1 ; c1ccc(-c2cccc(-c3c(-c4ccccc4)cc(-c4ccccc4)cc3-c3nc(-c4cccc5c4sc4ccccc45)nc(-c4cccc5c4sc4ccccc45)n3)c2)cc1]

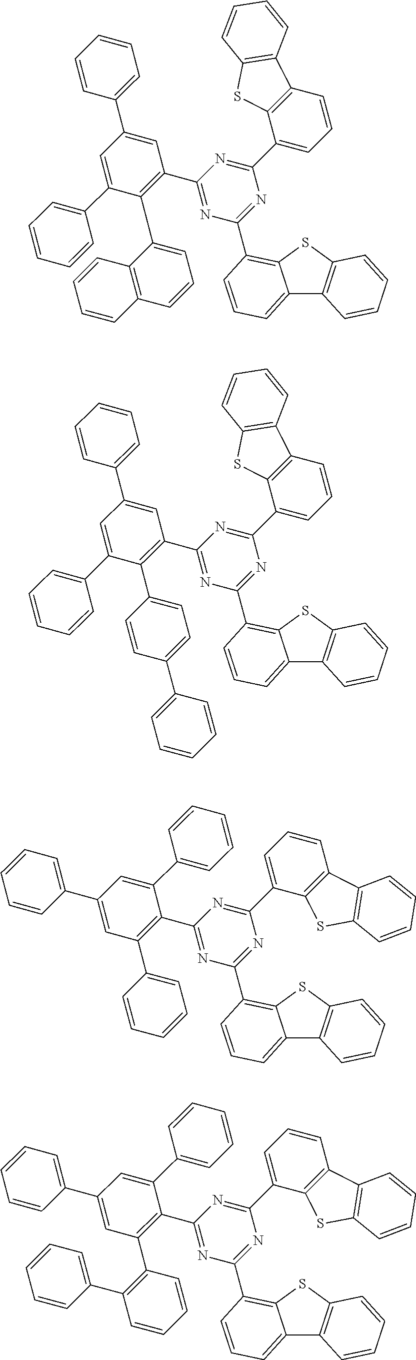 [SMILES: c1ccc(-c2cc(-c3ccccc3)c(-c3cccc4ccccc34)c(-c3nc(-c4cccc5c4sc4ccccc45)nc(-c4cccc5c4sc4ccccc45)n3)c2)cc1 ; c1ccc(-c2ccc(-c3c(-c4ccccc4)cc(-c4ccccc4)cc3-c3nc(-c4cccc5c4sc4ccccc45)nc(-c4cccc5c4sc4ccccc45)n3)cc2)cc1 ; c1ccc(-c2cc(-c3ccccc3)c(-c3nc(-c4cccc5c4sc4ccccc45)nc(-c4cccc5c4sc4ccccc45)n3)c(-c3ccccc3)c2)cc1 ; c1ccc(-c2cc(-c3ccccc3)c(-c3nc(-c4cccc5c4sc4ccccc45)nc(-c4cccc5c4sc4ccccc45)n3)c(-c3ccccc3-c3ccccc3)c2)cc1]

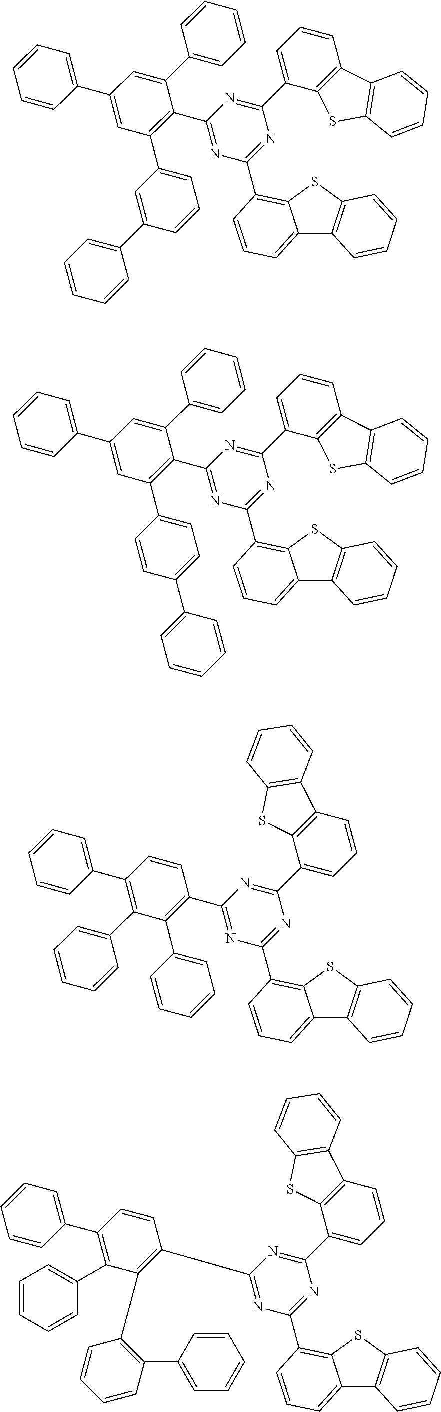 [SMILES: c1ccc(-c2cccc(-c3cc(-c4ccccc4)cc(-c4ccccc4)c3-c3nc(-c4cccc5c4sc4ccccc45)nc(-c4cccc5c4sc4ccccc45)n3)c2)cc1 ; c1ccc(-c2ccc(-c3cc(-c4ccccc4)cc(-c4ccccc4)c3-c3nc(-c4cccc5c4sc4ccccc45)nc(-c4cccc5c4sc4ccccc45)n3)cc2)cc1 ; c1ccc(-c2ccc(-c3nc(-c4cccc5c4sc4ccccc45)nc(-c4cccc5c4sc4ccccc45)n3)c(-c3ccccc3)c2-c2ccccc2)cc1 ; c1ccc(-c2ccccc2-c2c(-c3nc(-c4cccc5c4sc4ccccc45)nc(-c4cccc5c4sc4ccccc45)n3)ccc(-c3ccccc3)c2-c2ccccc2)cc1]

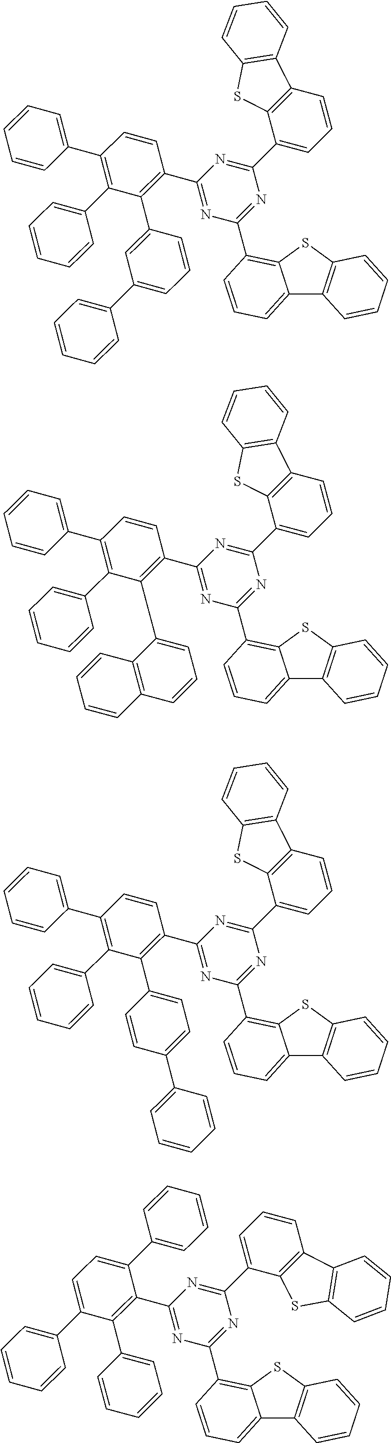

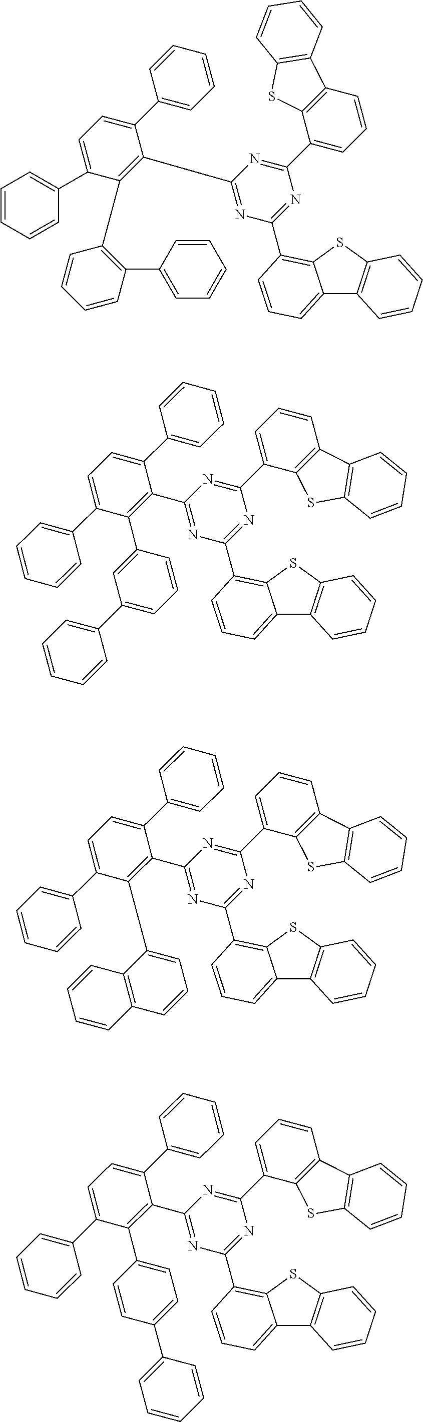 [SMILES: c1ccc(-c2ccccc2-c2c(-c3ccccc3)ccc(-c3ccccc3)c2-c2nc(-c3cccc4c3sc3ccccc34)nc(-c3cccc4c3sc3ccccc34)n2)cc1 ; c1ccc(-c2cccc(-c3c(-c4ccccc4)ccc(-c4ccccc4)c3-c3nc(-c4cccc5c4sc4ccccc45)nc(-c4cccc5c4sc4ccccc45)n3)c2)cc1 ; c1ccc(-c2ccc(-c3ccccc3)c(-c3cccc4ccccc34)c2-c2nc(-c3cccc4c3sc3ccccc34)nc(-c3cccc4c3sc3ccccc34)n2)cc1 ; c1ccc(-c2ccc(-c3c(-c4ccccc4)ccc(-c4ccccc4)c3-c3nc(-c4cccc5c4sc4ccccc45)nc(-c4cccc5c4sc4ccccc45)n3)cc2)cc1]

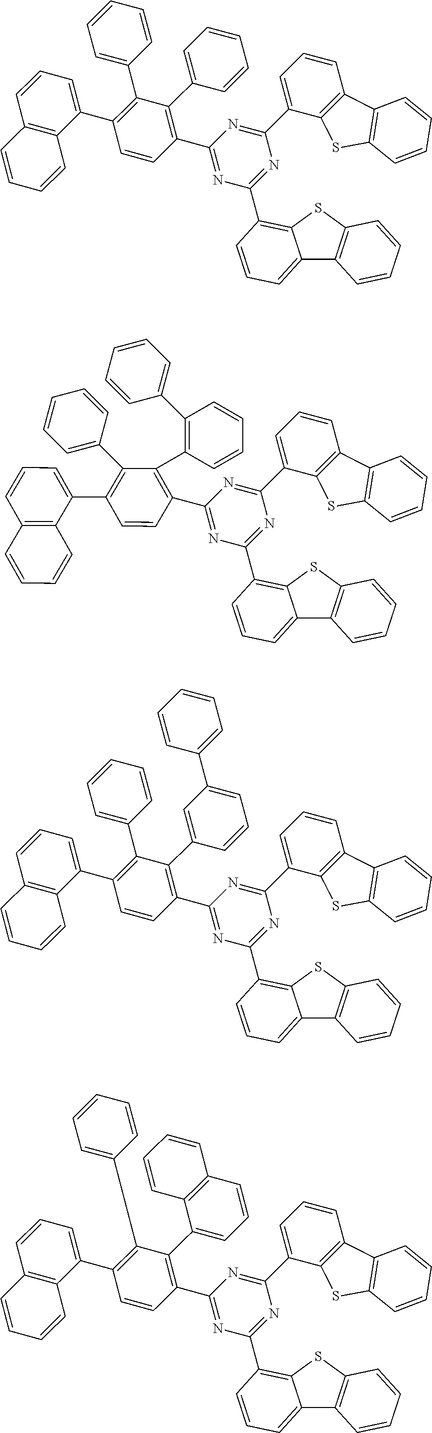 [SMILES: c1ccc(-c2c(-c3nc(-c4cccc5c4sc4ccccc45)nc(-c4cccc5c4sc4ccccc45)n3)ccc(-c3cccc4ccccc34)c2-c2ccccc2)cc1 ; c1ccc(-c2ccccc2-c2c(-c3nc(-c4cccc5c4sc4ccccc45)nc(-c4cccc5c4sc4ccccc45)n3)ccc(-c3cccc4ccccc34)c2-c2ccccc2)cc1 ; c1ccc(-c2cccc(-c3c(-c4nc(-c5cccc6c5sc5ccccc56)nc(-c5cccc6c5sc5ccccc56)n4)ccc(-c4cccc5ccccc45)c3-c3ccccc3)c2)cc1 ; c1ccc(-c2c(-c3cccc4ccccc34)ccc(-c3nc(-c4cccc5c4sc4ccccc45)nc(-c4cccc5c4sc4ccccc45)n3)c2-c2cccc3ccccc23)cc1]

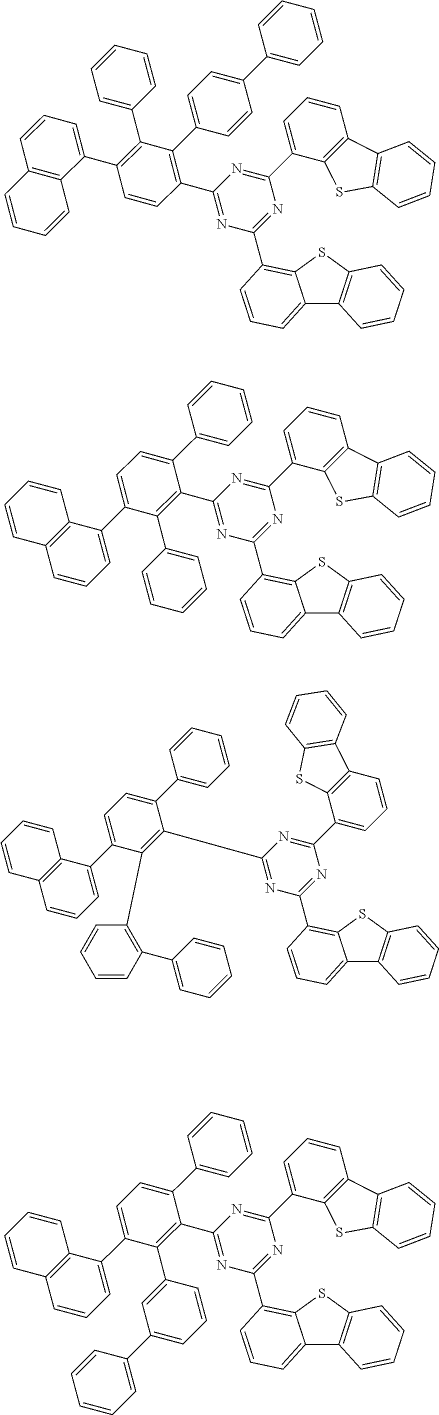 [SMILES: c1ccc(-c2ccc(-c3c(-c4nc(-c5cccc6c5sc5ccccc56)nc(-c5cccc6c5sc5ccccc56)n4)ccc(-c4cccc5ccccc45)c3-c3ccccc3)cc2)cc1 ; c1ccc(-c2ccc(-c3cccc4ccccc34)c(-c3ccccc3)c2-c2nc(-c3cccc4c3sc3ccccc34)nc(-c3cccc4c3sc3ccccc34)n2)cc1 ; c1ccc(-c2ccccc2-c2c(-c3cccc4ccccc34)ccc(-c3ccccc3)c2-c2nc(-c3cccc4c3sc3ccccc34)nc(-c3cccc4c3sc3ccccc34)n2)cc1 ; c1ccc(-c2cccc(-c3c(-c4cccc5ccccc45)ccc(-c4ccccc4)c3-c3nc(-c4cccc5c4sc4ccccc45)nc(-c4cccc5c4sc4ccccc45)n3)c2)cc1]

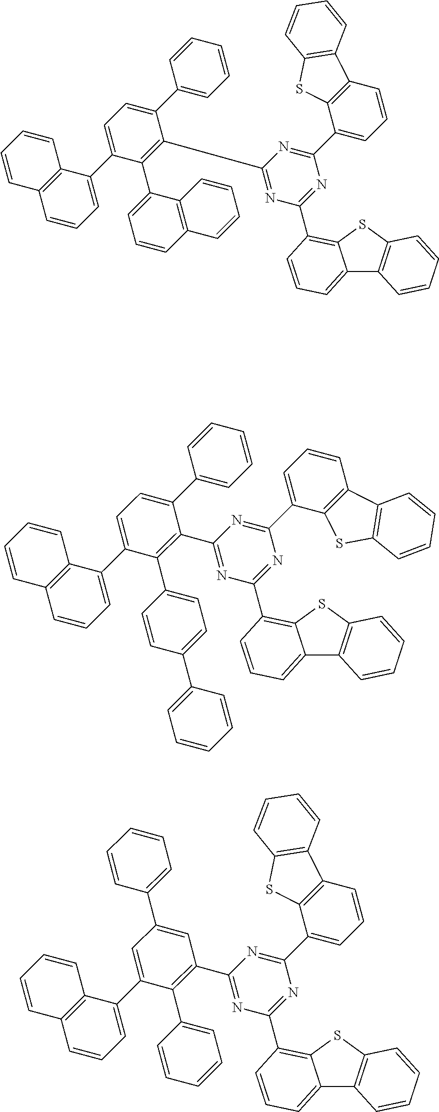 [SMILES: c1ccc(-c2ccc(-c3cccc4ccccc34)c(-c3cccc4ccccc34)c2-c2nc(-c3cccc4c3sc3ccccc34)nc(-c3cccc4c3sc3ccccc34)n2)cc1 ; c1ccc(-c2ccc(-c3c(-c4cccc5ccccc45)ccc(-c4ccccc4)c3-c3nc(-c4cccc5c4sc4ccccc45)nc(-c4cccc5c4sc4ccccc45)n3)cc2)cc1 ; c1ccc(-c2cc(-c3nc(-c4cccc5c4sc4ccccc45)nc(-c4cccc5c4sc4ccccc45)n3)c(-c3ccccc3)c(-c3cccc4ccccc34)c2)cc1]

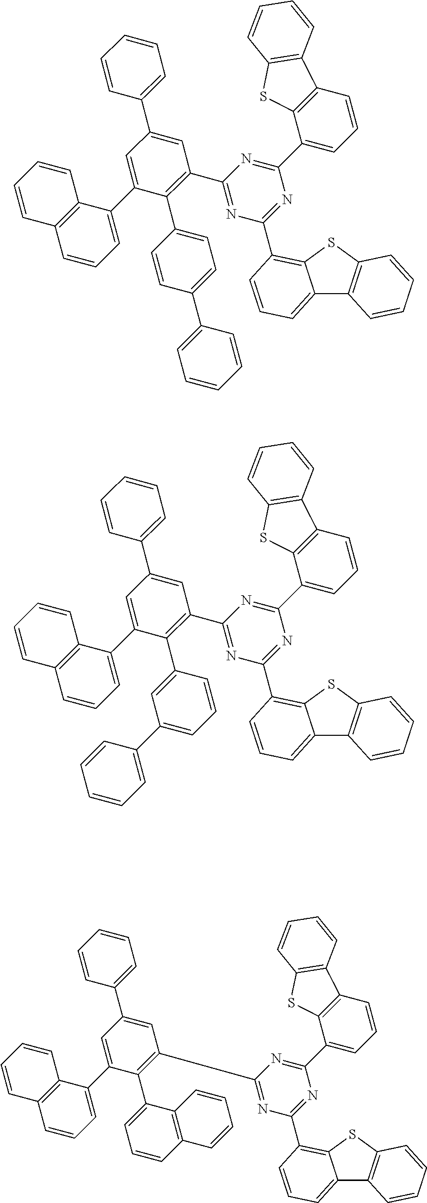 [SMILES: c1ccc(-c2ccc(-c3c(-c4nc(-c5cccc6c5sc5ccccc56)nc(-c5cccc6c5sc5ccccc56)n4)cc(-c4ccccc4)cc3-c3cccc4ccccc34)cc2)cc1 ; c1ccc(-c2cccc(-c3c(-c4nc(-c5cccc6c5sc5ccccc56)nc(-c5cccc6c5sc5ccccc56)n4)cc(-c4ccccc4)cc3-c3cccc4ccccc34)c2)cc1 ; c1ccc(-c2cc(-c3nc(-c4cccc5c4sc4ccccc45)nc(-c4cccc5c4sc4ccccc45)n3)c(-c3cccc4ccccc34)c(-c3cccc4ccccc34)c2)cc1]

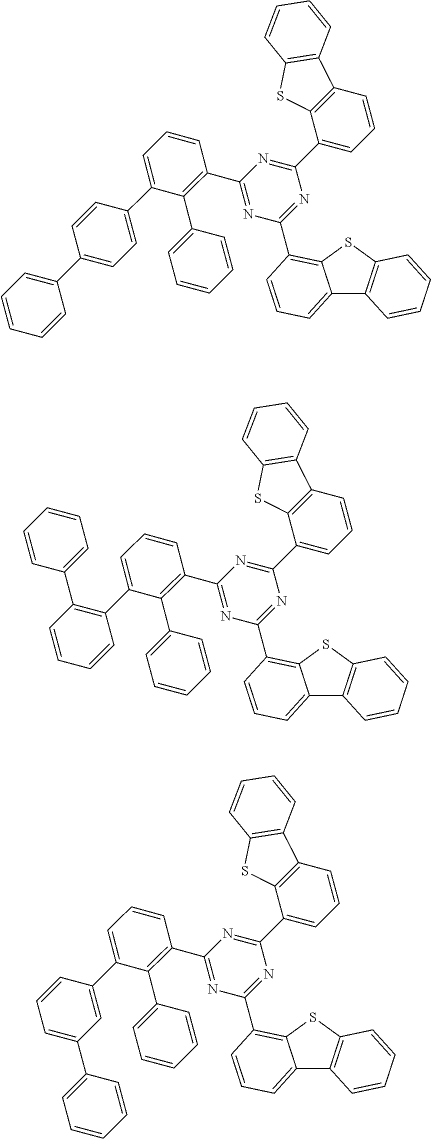 [SMILES: c1ccc(-c2ccc(-c3cccc(-c4nc(-c5cccc6c5sc5ccccc56)nc(-c5cccc6c5sc5ccccc56)n4)c3-c3ccccc3)cc2)cc1 ; c1ccc(-c2ccccc2-c2cccc(-c3nc(-c4cccc5c4sc4ccccc45)nc(-c4cccc5c4sc4ccccc45)n3)c2-c2ccccc2)cc1 ; c1ccc(-c2cccc(-c3cccc(-c4nc(-c5cccc6c5sc5ccccc56)nc(-c5cccc6c5sc5ccccc56)n4)c3-c3ccccc3)c2)cc1]

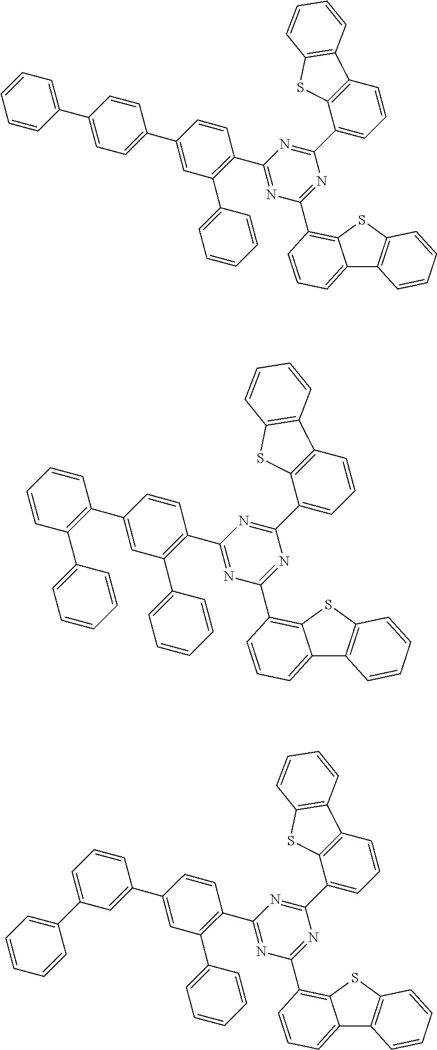 [SMILES: c1ccc(-c2ccc(-c3ccc(-c4nc(-c5cccc6c5sc5ccccc56)nc(-c5cccc6c5sc5ccccc56)n4)c(-c4ccccc4)c3)cc2)cc1 ; c1ccc(-c2ccccc2-c2ccc(-c3nc(-c4cccc5c4sc4ccccc45)nc(-c4cccc5c4sc4ccccc45)n3)c(-c3ccccc3)c2)cc1 ; c1ccc(-c2cccc(-c3ccc(-c4nc(-c5cccc6c5sc5ccccc56)nc(-c5cccc6c5sc5ccccc56)n4)c(-c4ccccc4)c3)c2)cc1]

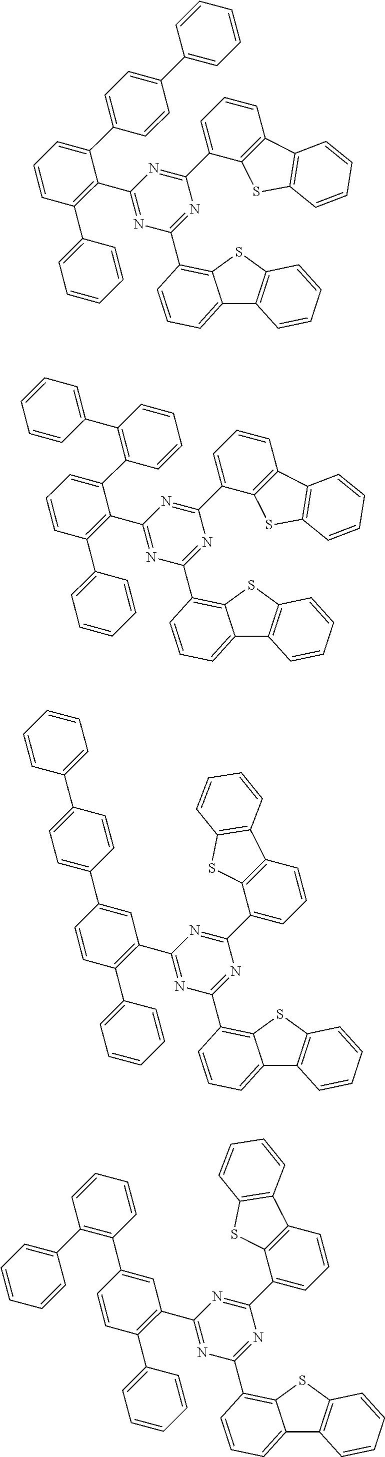 [SMILES: c1ccc(-c2ccc(-c3cccc(-c4ccccc4)c3-c3nc(-c4cccc5c4sc4ccccc45)nc(-c4cccc5c4sc4ccccc45)n3)cc2)cc1 ; c1ccc(-c2ccccc2-c2cccc(-c3ccccc3)c2-c2nc(-c3cccc4c3sc3ccccc34)nc(-c3cccc4c3sc3ccccc34)n2)cc1 ; c1ccc(-c2ccc(-c3ccc(-c4ccccc4)c(-c4nc(-c5cccc6c5sc5ccccc56)nc(-c5cccc6c5sc5ccccc56)n4)c3)cc2)cc1 ; c1ccc(-c2ccccc2-c2ccc(-c3ccccc3)c(-c3nc(-c4cccc5c4sc4ccccc45)nc(-c4cccc5c4sc4ccccc45)n3)c2)cc1]

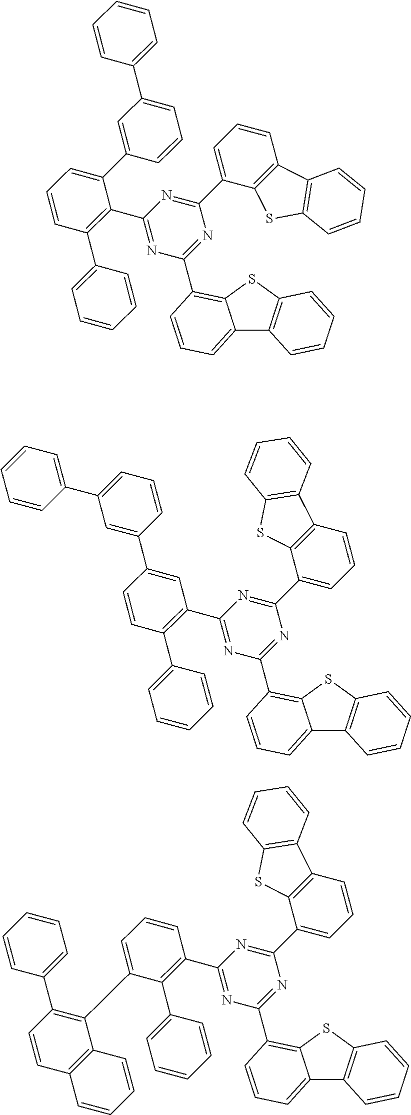

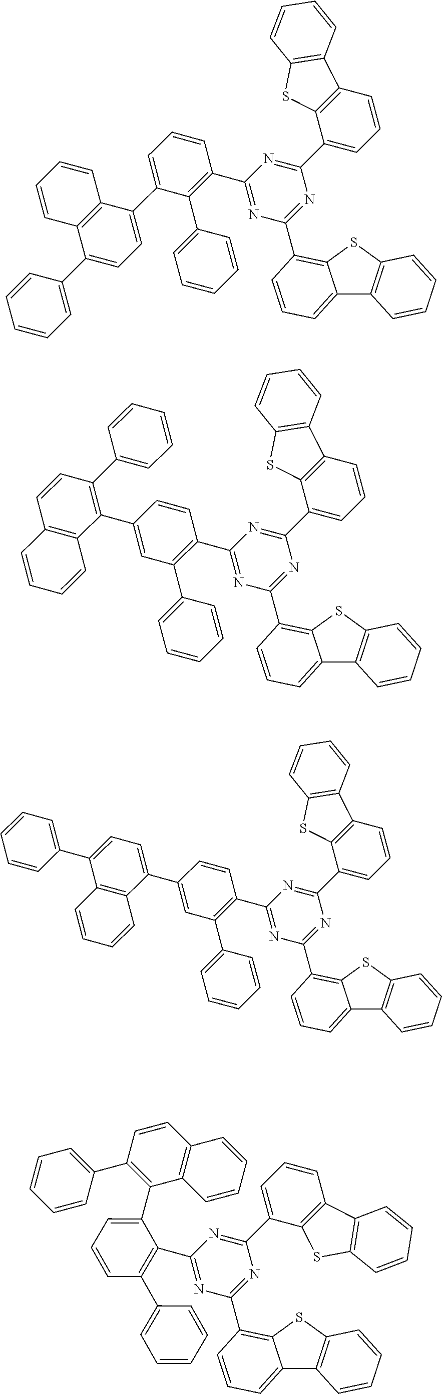 [SMILES: c1ccc(-c2c(-c3nc(-c4cccc5c4sc4ccccc45)nc(-c4cccc5c4sc4ccccc45)n3)cccc2-c2ccc(-c3ccccc3)c3ccccc23)cc1 ; c1ccc(-c2cc(-c3c(-c4ccccc4)ccc4ccccc34)ccc2-c2nc(-c3cccc4c3sc3ccccc34)nc(-c3cccc4c3sc3ccccc34)n2)cc1 ; c1ccc(-c2cc(-c3ccc(-c4ccccc4)c4ccccc34)ccc2-c2nc(-c3cccc4c3sc3ccccc34)nc(-c3cccc4c3sc3ccccc34)n2)cc1 ; c1ccc(-c2cccc(-c3c(-c4ccccc4)ccc4ccccc34)c2-c2nc(-c3cccc4c3sc3ccccc34)nc(-c3cccc4c3sc3ccccc34)n2)cc1]

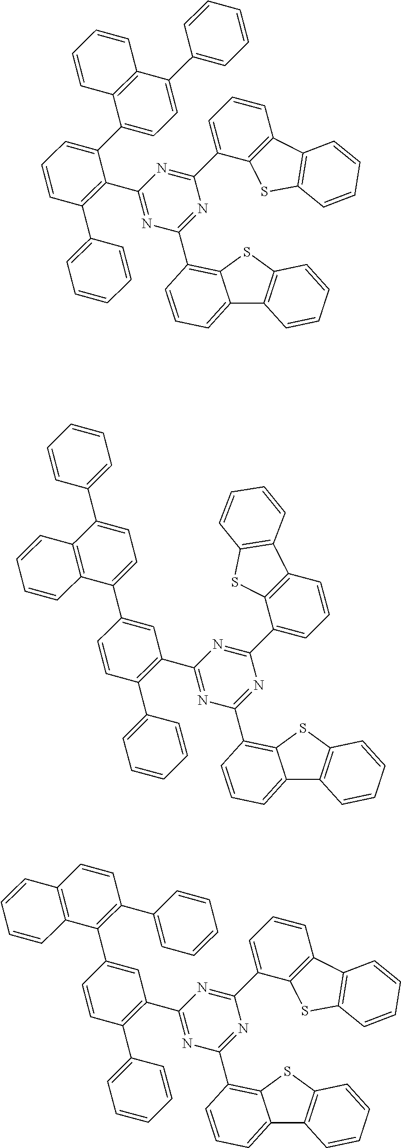 [SMILES: c1ccc(-c2cccc(-c3ccc(-c4ccccc4)c4ccccc34)c2-c2nc(-c3cccc4c3sc3ccccc34)nc(-c3cccc4c3sc3ccccc34)n2)cc1 ; c1ccc(-c2ccc(-c3ccc(-c4ccccc4)c4ccccc34)cc2-c2nc(-c3cccc4c3sc3ccccc34)nc(-c3cccc4c3sc3ccccc34)n2)cc1 ; c1ccc(-c2ccc(-c3c(-c4ccccc4)ccc4ccccc34)cc2-c2nc(-c3cccc4c3sc3ccccc34)nc(-c3cccc4c3sc3ccccc34)n2)cc1]

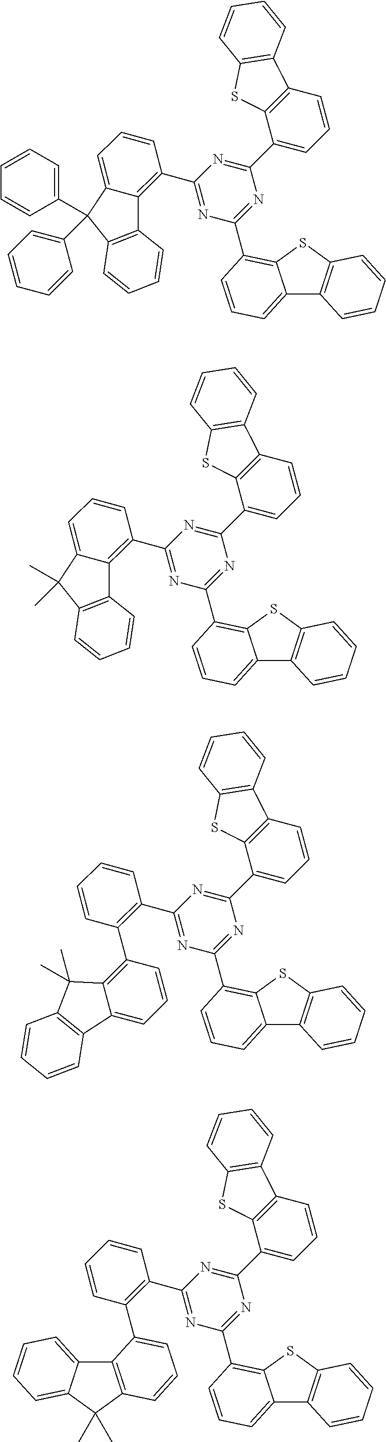 [SMILES: c1ccc(C2(c3ccccc3)c3ccccc3-c3c(-c4nc(-c5cccc6c5sc5ccccc56)nc(-c5cccc6c5sc5ccccc56)n4)cccc32)cc1 ; CC1(C)c2ccccc2-c2c(-c3nc(-c4cccc5c4sc4ccccc45)nc(-c4cccc5c4sc4ccccc45)n3)cccc21 ; CC1(C)c2ccccc2-c2cccc(-c3ccccc3-c3nc(-c4cccc5c4sc4ccccc45)nc(-c4cccc5c4sc4ccccc45)n3)c21 ; CC1(C)c2ccccc2-c2c(-c3ccccc3-c3nc(-c4cccc5c4sc4ccccc45)nc(-c4cccc5c4sc4ccccc45)n3)cccc21]

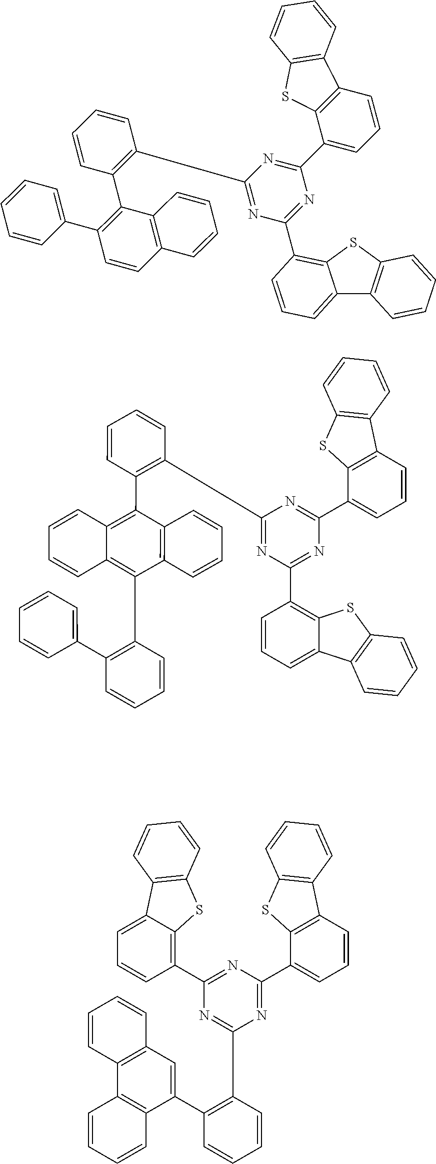

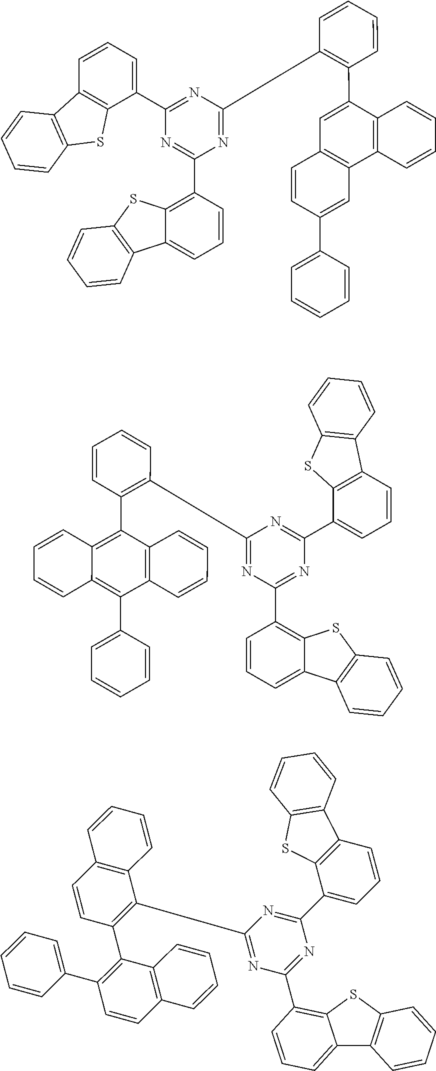 [SMILES: c1ccc(-c2ccc3cc(-c4ccccc4-c4nc(-c5cccc6c5sc5ccccc56)nc(-c5cccc6c5sc5ccccc56)n4)c4ccccc4c3c2)cc1 ; c1ccc(-c2c3ccccc3c(-c3ccccc3-c3nc(-c4cccc5c4sc4ccccc45)nc(-c4cccc5c4sc4ccccc45)n3)c3ccccc23)cc1 ; c1ccc(-c2ccc3ccccc3c2-c2ccc3ccccc3c2-c2nc(-c3cccc4c3sc3ccccc34)nc(-c3cccc4c3sc3ccccc34)n2)cc1]

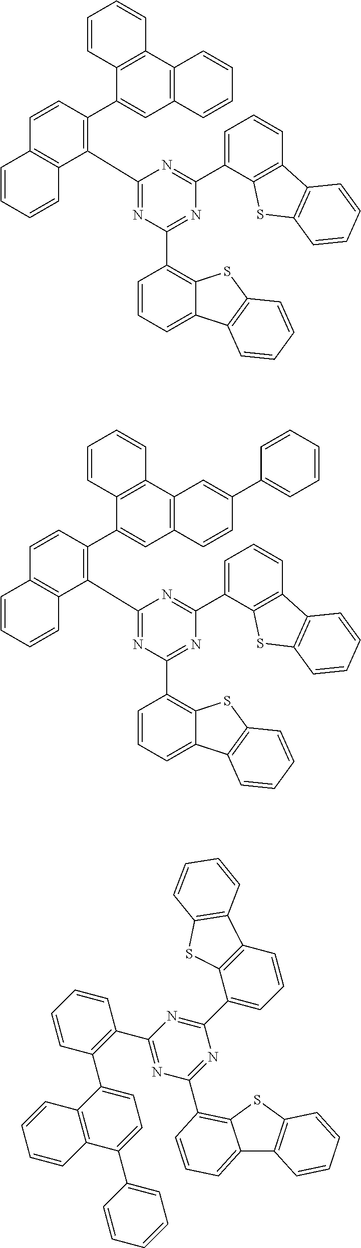 [SMILES: c1ccc2c(-c3nc(-c4cccc5c4sc4ccccc45)nc(-c4cccc5c4sc4ccccc45)n3)c(-c3cc4ccccc4c4ccccc34)ccc2c1 ; c1ccc(-c2ccc3cc(-c4ccc5ccccc5c4-c4nc(-c5cccc6c5sc5ccccc56)nc(-c5cccc6c5sc5ccccc56)n4)c4ccccc4c3c2)cc1 ; c1ccc(-c2ccc(-c3ccccc3-c3nc(-c4cccc5c4sc4ccccc45)nc(-c4cccc5c4sc4ccccc45)n3)c3ccccc23)cc1]

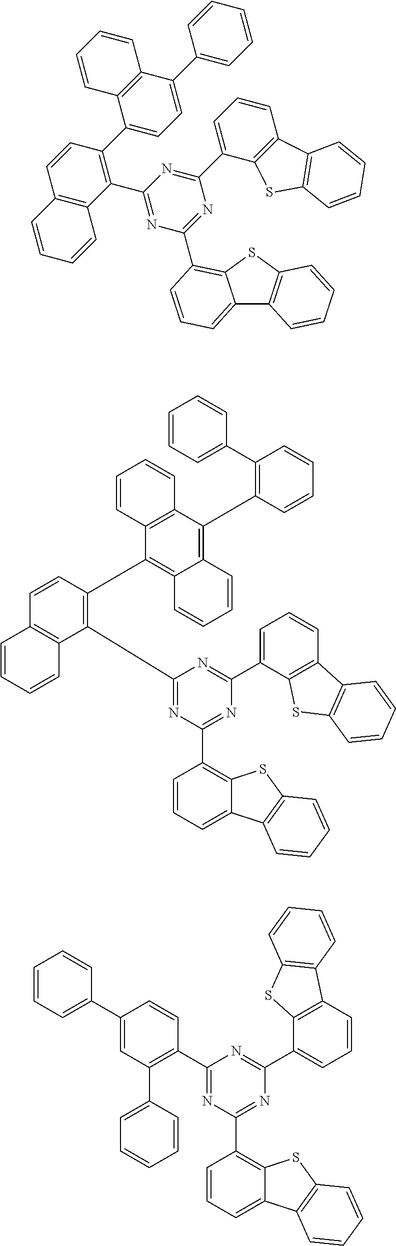 [SMILES: c1ccc(-c2ccc(-c3ccc4ccccc4c3-c3nc(-c4cccc5c4sc4ccccc45)nc(-c4cccc5c4sc4ccccc45)n3)c3ccccc23)cc1 ; c1ccc(-c2ccccc2-c2c3ccccc3c(-c3ccc4ccccc4c3-c3nc(-c4cccc5c4sc4ccccc45)nc(-c4cccc5c4sc4ccccc45)n3)c3ccccc23)cc1 ; c1ccc(-c2ccc(-c3nc(-c4cccc5c4sc4ccccc45)nc(-c4cccc5c4sc4ccccc45)n3)c(-c3ccccc3)c2)cc1]

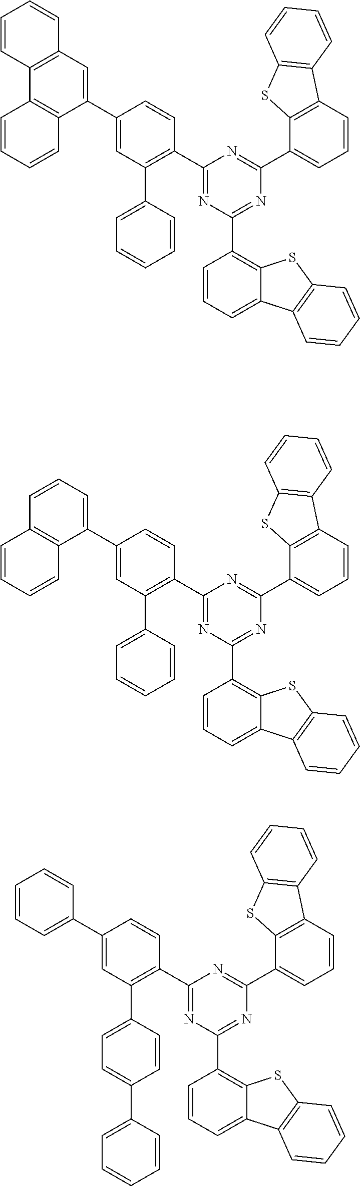 [SMILES: c1ccc(-c2cc(-c3cc4ccccc4c4ccccc34)ccc2-c2nc(-c3cccc4c3sc3ccccc34)nc(-c3cccc4c3sc3ccccc34)n2)cc1 ; c1ccc(-c2cc(-c3cccc4ccccc34)ccc2-c2nc(-c3cccc4c3sc3ccccc34)nc(-c3cccc4c3sc3ccccc34)n2)cc1 ; c1ccc(-c2ccc(-c3cc(-c4ccccc4)ccc3-c3nc(-c4cccc5c4sc4ccccc45)nc(-c4cccc5c4sc4ccccc45)n3)cc2)cc1]

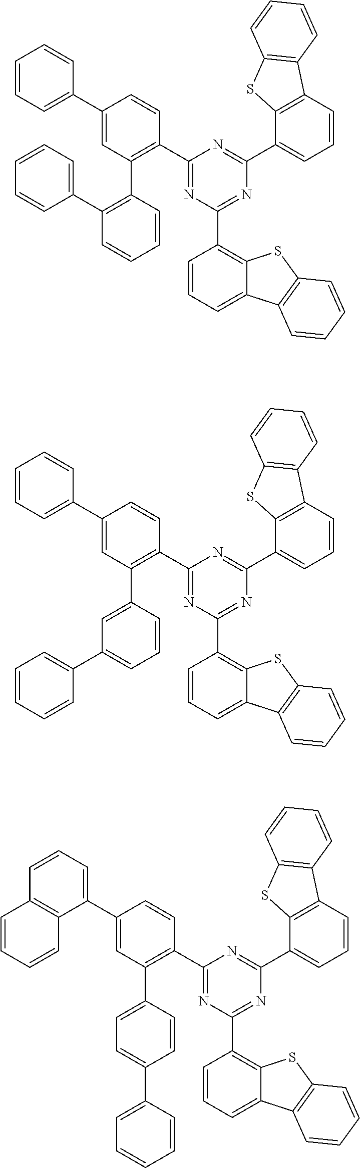 [SMILES: c1ccc(-c2ccc(-c3nc(-c4cccc5c4sc4ccccc45)nc(-c4cccc5c4sc4ccccc45)n3)c(-c3ccccc3-c3ccccc3)c2)cc1 ; c1ccc(-c2cccc(-c3cc(-c4ccccc4)ccc3-c3nc(-c4cccc5c4sc4ccccc45)nc(-c4cccc5c4sc4ccccc45)n3)c2)cc1 ; c1ccc(-c2ccc(-c3cc(-c4cccc5ccccc45)ccc3-c3nc(-c4cccc5c4sc4ccccc45)nc(-c4cccc5c4sc4ccccc45)n3)cc2)cc1]

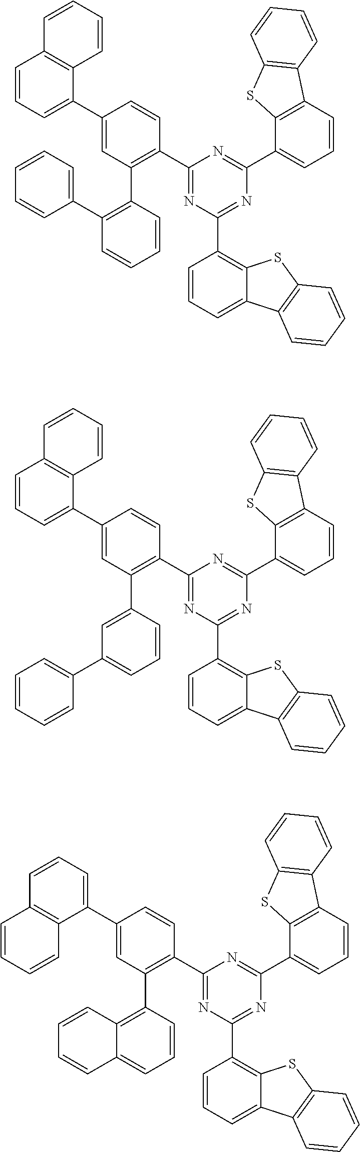 [SMILES: c1ccc(-c2ccccc2-c2cc(-c3cccc4ccccc34)ccc2-c2nc(-c3cccc4c3sc3ccccc34)nc(-c3cccc4c3sc3ccccc34)n2)cc1 ; c1ccc(-c2cccc(-c3cc(-c4cccc5ccccc45)ccc3-c3nc(-c4cccc5c4sc4ccccc45)nc(-c4cccc5c4sc4ccccc45)n3)c2)cc1 ; c1ccc2c(-c3ccc(-c4nc(-c5cccc6c5sc5ccccc56)nc(-c5cccc6c5sc5ccccc56)n4)c(-c4cccc5ccccc45)c3)cccc2c1]

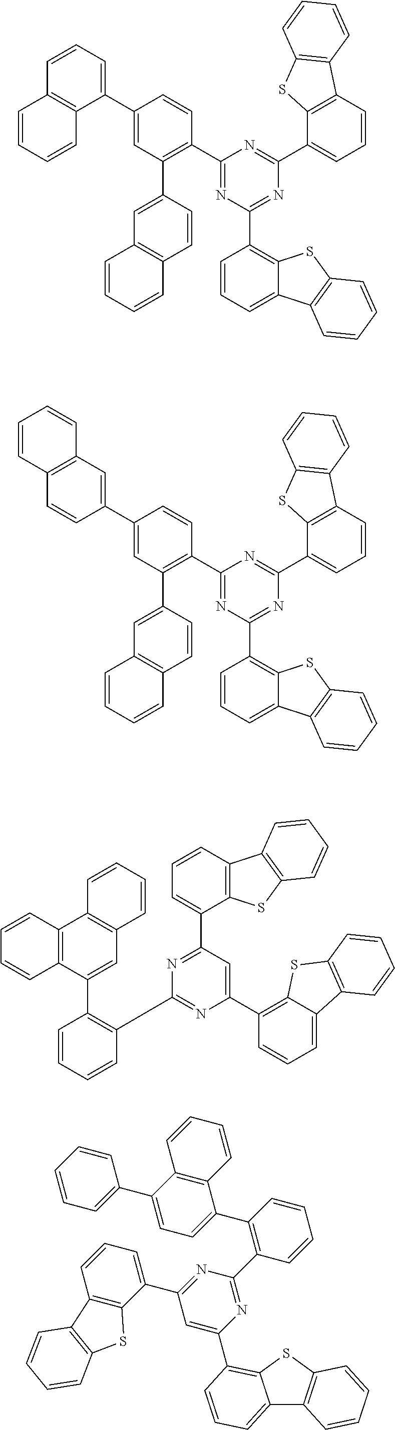 [SMILES: c1ccc2cc(-c3cc(-c4cccc5ccccc45)ccc3-c3nc(-c4cccc5c4sc4ccccc45)nc(-c4cccc5c4sc4ccccc45)n3)ccc2c1 ; c1ccc2cc(-c3ccc(-c4nc(-c5cccc6c5sc5ccccc56)nc(-c5cccc6c5sc5ccccc56)n4)c(-c4ccc5ccccc5c4)c3)ccc2c1 ; c1ccc(-c2cc3ccccc3c3ccccc23)c(-c2nc(-c3cccc4c3sc3ccccc34)cc(-c3cccc4c3sc3ccccc34)n2)c1 ; c1ccc(-c2ccc(-c3ccccc3-c3nc(-c4cccc5c4sc4ccccc45)cc(-c4cccc5c4sc4ccccc45)n3)c3ccccc23)cc1]

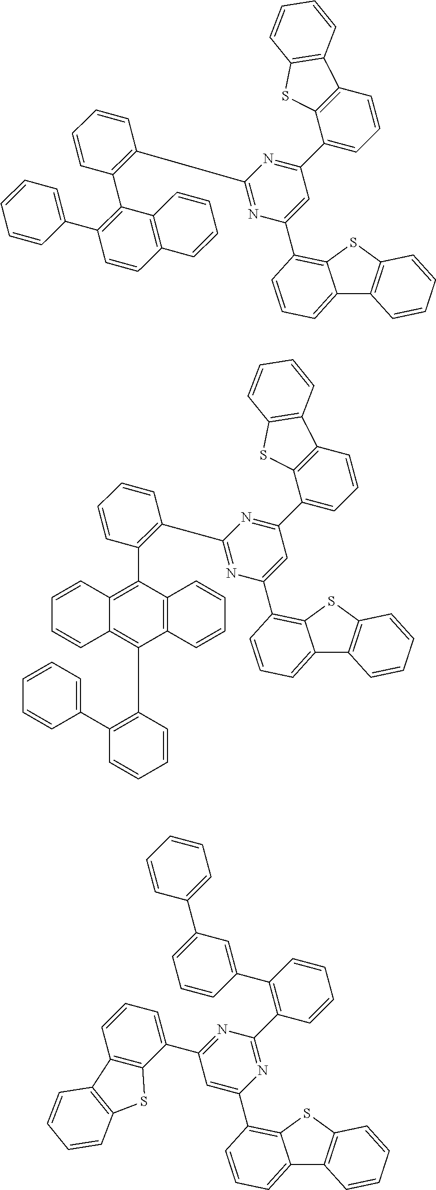 [SMILES: c1ccc(-c2ccc3ccccc3c2-c2ccccc2-c2nc(-c3cccc4c3sc3ccccc34)cc(-c3cccc4c3sc3ccccc34)n2)cc1 ; c1ccc(-c2ccccc2-c2c3ccccc3c(-c3ccccc3-c3nc(-c4cccc5c4sc4ccccc45)cc(-c4cccc5c4sc4ccccc45)n3)c3ccccc23)cc1 ; c1ccc(-c2cccc(-c3ccccc3-c3nc(-c4cccc5c4sc4ccccc45)cc(-c4cccc5c4sc4ccccc45)n3)c2)cc1]

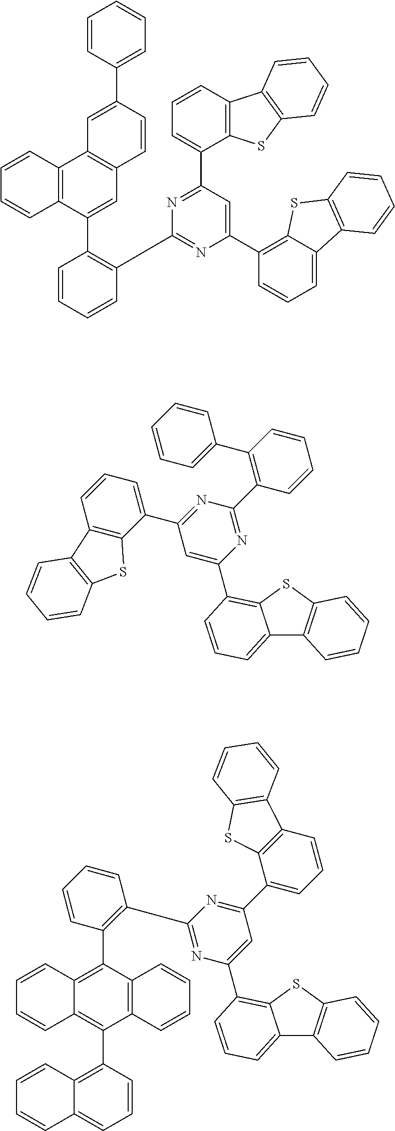 [SMILES: c1ccc(-c2ccc3cc(-c4ccccc4-c4nc(-c5cccc6c5sc5ccccc56)cc(-c5cccc6c5sc5ccccc56)n4)c4ccccc4c3c2)cc1 ; c1ccc(-c2ccccc2-c2nc(-c3cccc4c3sc3ccccc34)cc(-c3cccc4c3sc3ccccc34)n2)cc1 ; c1ccc(-c2c3ccccc3c(-c3cccc4ccccc34)c3ccccc23)c(-c2nc(-c3cccc4c3sc3ccccc34)cc(-c3cccc4c3sc3ccccc34)n2)c1]

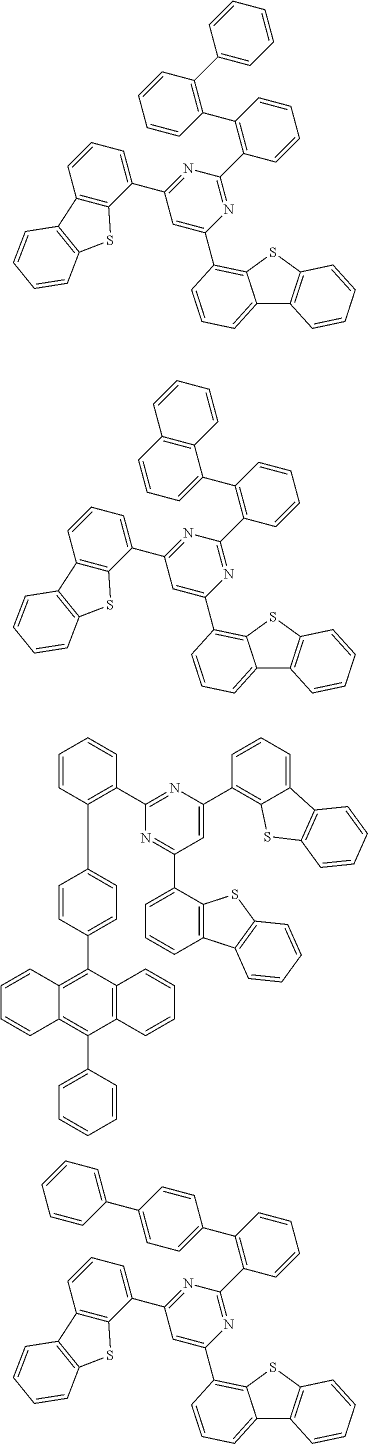

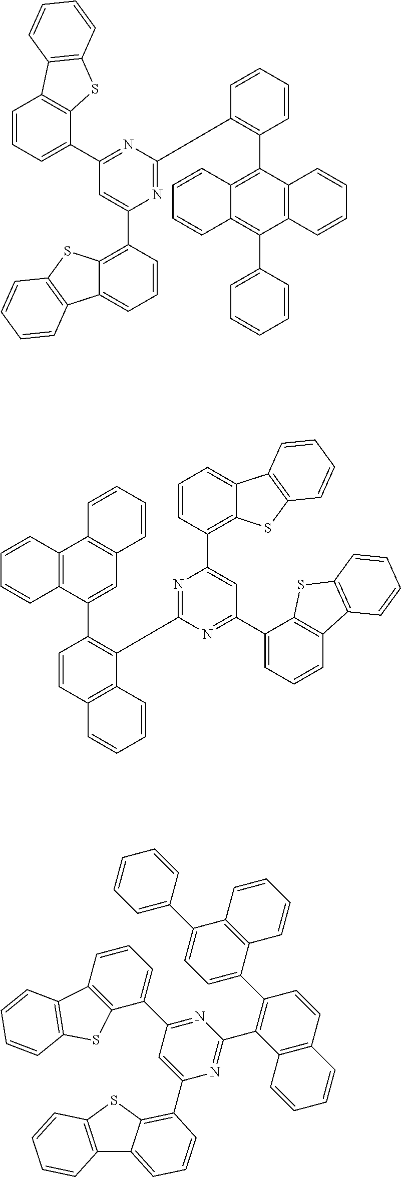 [SMILES: c1ccc(-c2c3ccccc3c(-c3ccccc3-c3nc(-c4cccc5c4sc4ccccc45)cc(-c4cccc5c4sc4ccccc45)n3)c3ccccc23)cc1 ; c1ccc2c(-c3nc(-c4cccc5c4sc4ccccc45)cc(-c4cccc5c4sc4ccccc45)n3)c(-c3cc4ccccc4c4ccccc34)ccc2c1 ; c1ccc(-c2ccc(-c3ccc4ccccc4c3-c3nc(-c4cccc5c4sc4ccccc45)cc(-c4cccc5c4sc4ccccc45)n3)c3ccccc23)cc1]

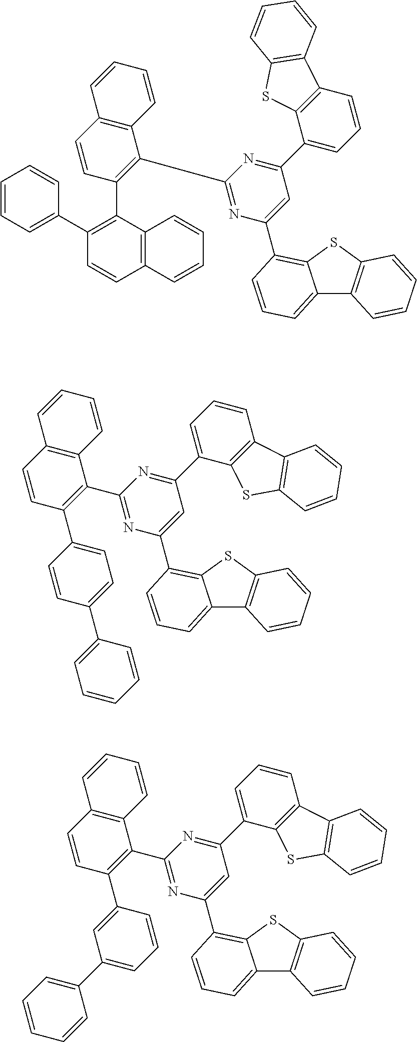 [SMILES: c1ccc(-c2ccc3ccccc3c2-c2ccc3ccccc3c2-c2nc(-c3cccc4c3sc3ccccc34)cc(-c3cccc4c3sc3ccccc34)n2)cc1 ; c1ccc(-c2ccc(-c3ccc4ccccc4c3-c3nc(-c4cccc5c4sc4ccccc45)cc(-c4cccc5c4sc4ccccc45)n3)cc2)cc1 ; c1ccc(-c2cccc(-c3ccc4ccccc4c3-c3nc(-c4cccc5c4sc4ccccc45)cc(-c4cccc5c4sc4ccccc45)n3)c2)cc1]

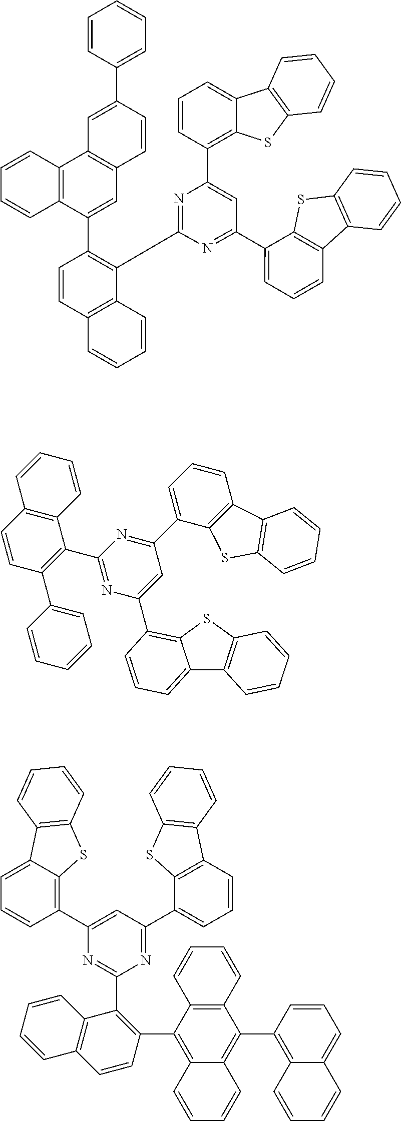 [SMILES: c1ccc(-c2ccc3cc(-c4ccc5ccccc5c4-c4nc(-c5cccc6c5sc5ccccc56)cc(-c5cccc6c5sc5ccccc56)n4)c4ccccc4c3c2)cc1 ; c1ccc(-c2ccc3ccccc3c2-c2nc(-c3cccc4c3sc3ccccc34)cc(-c3cccc4c3sc3ccccc34)n2)cc1 ; c1ccc2c(-c3c4ccccc4c(-c4ccc5ccccc5c4-c4nc(-c5cccc6c5sc5ccccc56)cc(-c5cccc6c5sc5ccccc56)n4)c4ccccc34)cccc2c1]

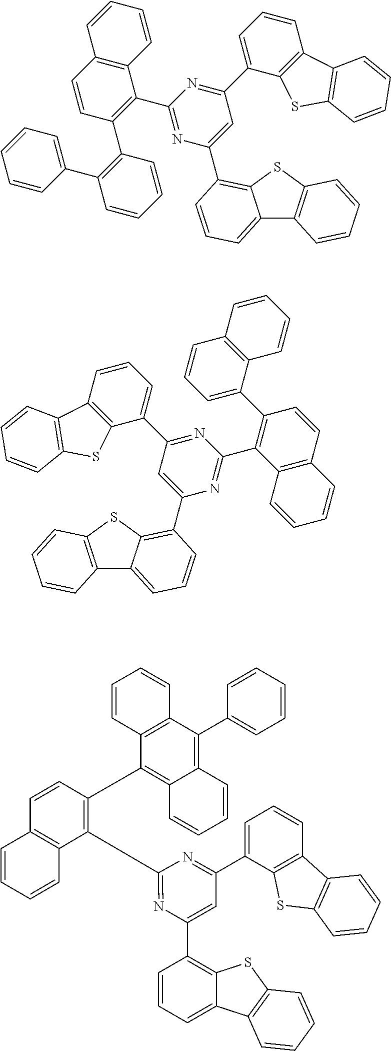 [SMILES: c1ccc(-c2ccccc2-c2ccc3ccccc3c2-c2nc(-c3cccc4c3sc3ccccc34)cc(-c3cccc4c3sc3ccccc34)n2)cc1 ; c1ccc2c(-c3ccc4ccccc4c3-c3nc(-c4cccc5c4sc4ccccc45)cc(-c4cccc5c4sc4ccccc45)n3)cccc2c1 ; c1ccc(-c2c3ccccc3c(-c3ccc4ccccc4c3-c3nc(-c4cccc5c4sc4ccccc45)cc(-c4cccc5c4sc4ccccc45)n3)c3ccccc23)cc1]

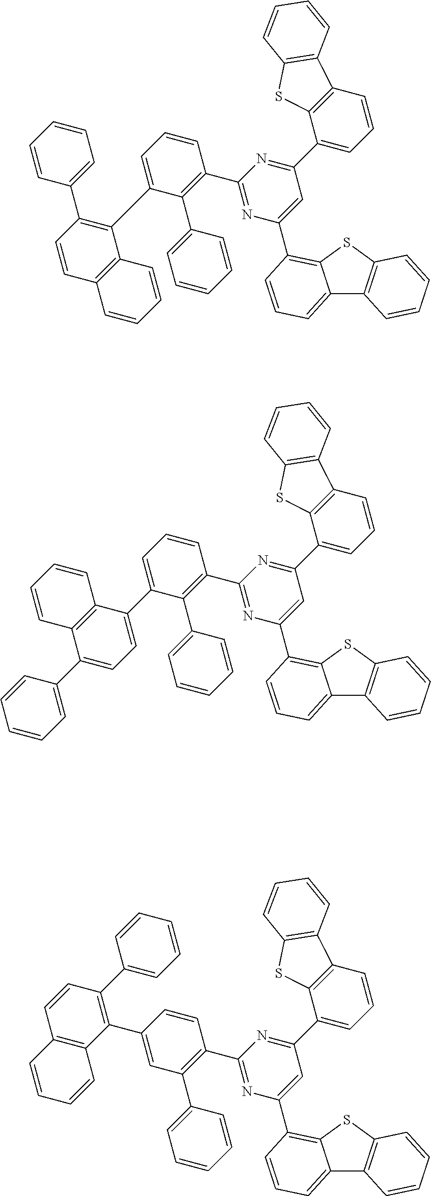 [SMILES: c1ccc(-c2ccc3ccccc3c2-c2cccc(-c3nc(-c4cccc5c4sc4ccccc45)cc(-c4cccc5c4sc4ccccc45)n3)c2-c2ccccc2)cc1 ; c1ccc(-c2c(-c3nc(-c4cccc5c4sc4ccccc45)cc(-c4cccc5c4sc4ccccc45)n3)cccc2-c2ccc(-c3ccccc3)c3ccccc23)cc1 ; c1ccc(-c2cc(-c3c(-c4ccccc4)ccc4ccccc34)ccc2-c2nc(-c3cccc4c3sc3ccccc34)cc(-c3cccc4c3sc3ccccc34)n2)cc1]

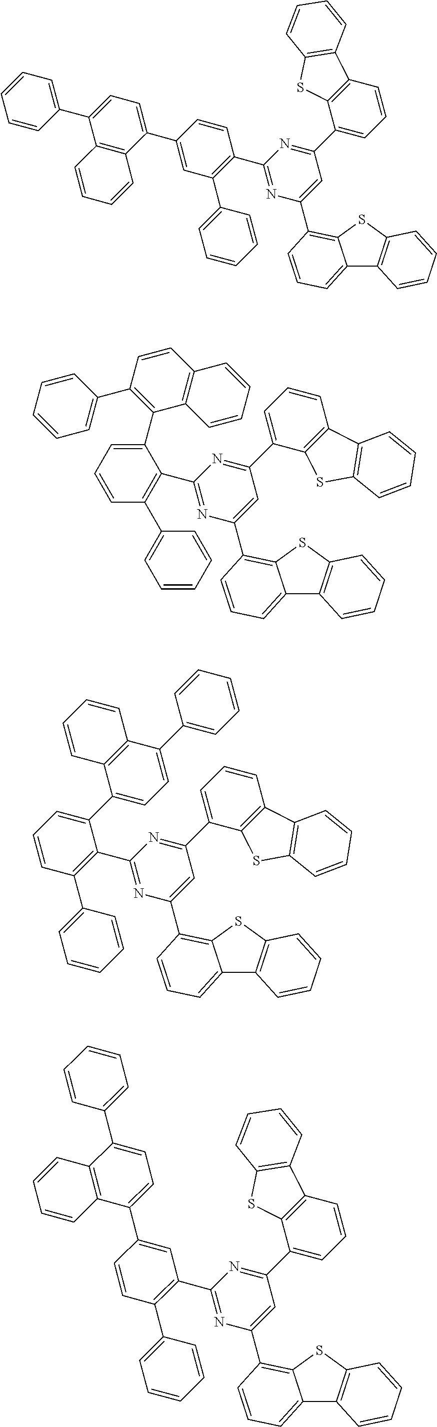 [SMILES: c1ccc(-c2cc(-c3ccc(-c4ccccc4)c4ccccc34)ccc2-c2nc(-c3cccc4c3sc3ccccc34)cc(-c3cccc4c3sc3ccccc34)n2)cc1 ; c1ccc(-c2cccc(-c3c(-c4ccccc4)ccc4ccccc34)c2-c2nc(-c3cccc4c3sc3ccccc34)cc(-c3cccc4c3sc3ccccc34)n2)cc1 ; c1ccc(-c2cccc(-c3ccc(-c4ccccc4)c4ccccc34)c2-c2nc(-c3cccc4c3sc3ccccc34)cc(-c3cccc4c3sc3ccccc34)n2)cc1 ; c1ccc(-c2ccc(-c3ccc(-c4ccccc4)c4ccccc34)cc2-c2nc(-c3cccc4c3sc3ccccc34)cc(-c3cccc4c3sc3ccccc34)n2)cc1]

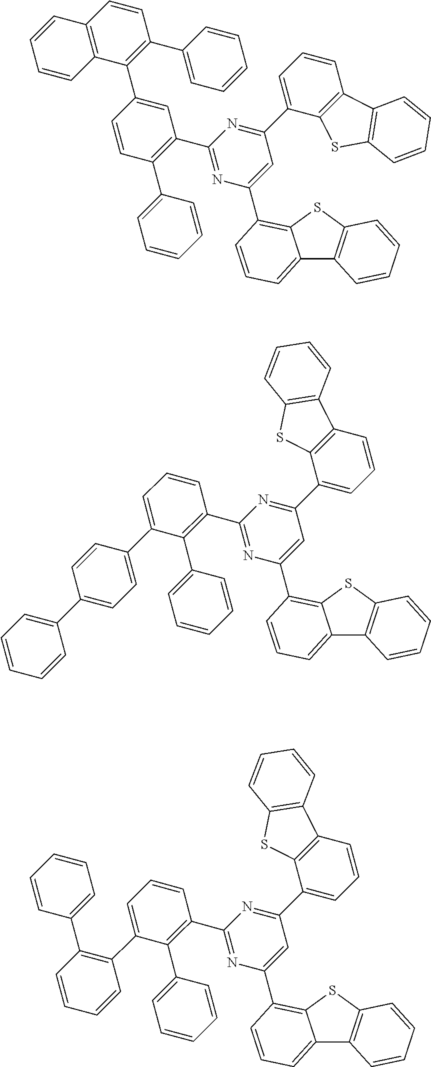 [SMILES: c1ccc(-c2ccc(-c3c(-c4ccccc4)ccc4ccccc34)cc2-c2nc(-c3cccc4c3sc3ccccc34)cc(-c3cccc4c3sc3ccccc34)n2)cc1 ; c1ccc(-c2ccc(-c3cccc(-c4nc(-c5cccc6c5sc5ccccc56)cc(-c5cccc6c5sc5ccccc56)n4)c3-c3ccccc3)cc2)cc1 ; c1ccc(-c2ccccc2-c2cccc(-c3nc(-c4cccc5c4sc4ccccc45)cc(-c4cccc5c4sc4ccccc45)n3)c2-c2ccccc2)cc1]

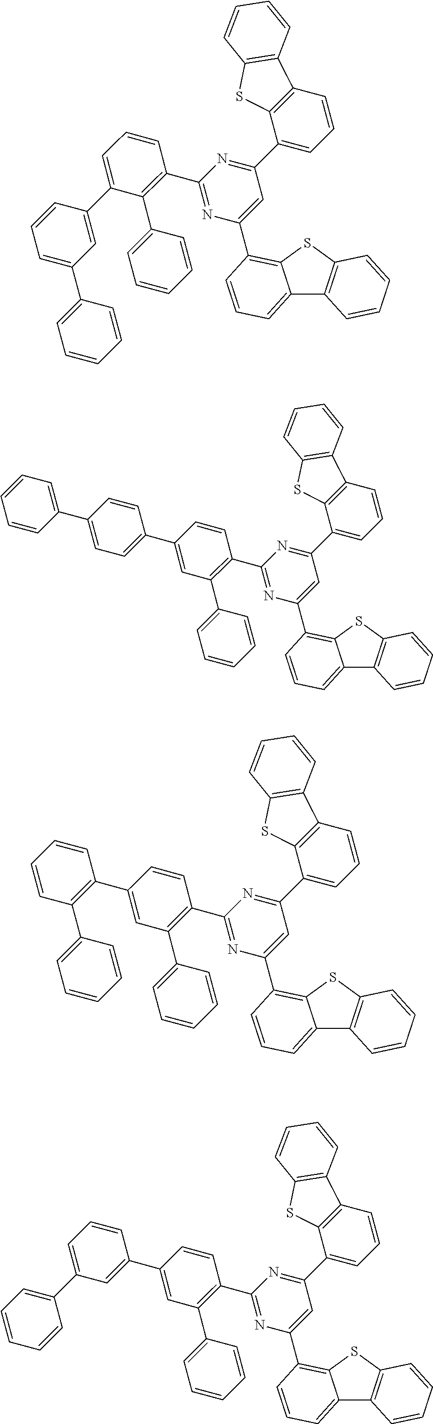 [SMILES: c1ccc(-c2cccc(-c3cccc(-c4nc(-c5cccc6c5sc5ccccc56)cc(-c5cccc6c5sc5ccccc56)n4)c3-c3ccccc3)c2)cc1 ; c1ccc(-c2ccc(-c3ccc(-c4nc(-c5cccc6c5sc5ccccc56)cc(-c5cccc6c5sc5ccccc56)n4)c(-c4ccccc4)c3)cc2)cc1 ; c1ccc(-c2ccccc2-c2ccc(-c3nc(-c4cccc5c4sc4ccccc45)cc(-c4cccc5c4sc4ccccc45)n3)c(-c3ccccc3)c2)cc1 ; c1ccc(-c2cccc(-c3ccc(-c4nc(-c5cccc6c5sc5ccccc56)cc(-c5cccc6c5sc5ccccc56)n4)c(-c4ccccc4)c3)c2)cc1]

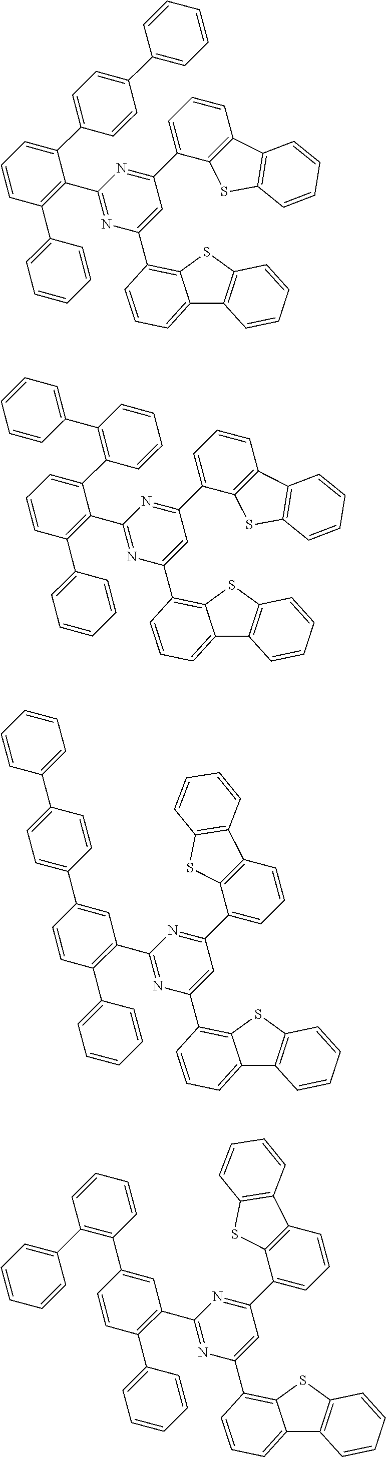 [SMILES: c1ccc(-c2ccc(-c3cccc(-c4ccccc4)c3-c3nc(-c4cccc5c4sc4ccccc45)cc(-c4cccc5c4sc4ccccc45)n3)cc2)cc1 ; c1ccc(-c2ccccc2-c2cccc(-c3ccccc3)c2-c2nc(-c3cccc4c3sc3ccccc34)cc(-c3cccc4c3sc3ccccc34)n2)cc1 ; c1ccc(-c2ccc(-c3ccc(-c4ccccc4)c(-c4nc(-c5cccc6c5sc5ccccc56)cc(-c5cccc6c5sc5ccccc56)n4)c3)cc2)cc1 ; c1ccc(-c2ccccc2-c2ccc(-c3ccccc3)c(-c3nc(-c4cccc5c4sc4ccccc45)cc(-c4cccc5c4sc4ccccc45)n3)c2)cc1]

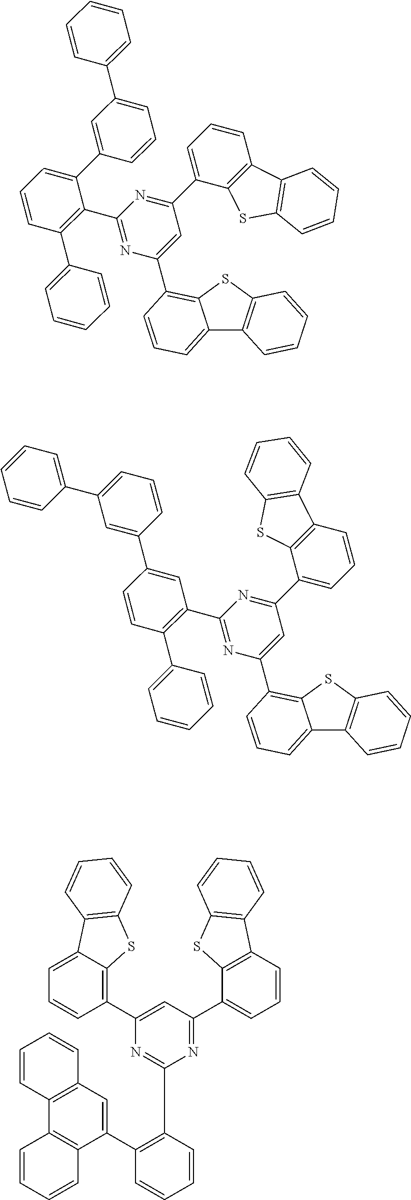 [SMILES: c1ccc(-c2cccc(-c3cccc(-c4ccccc4)c3-c3nc(-c4cccc5c4sc4ccccc45)cc(-c4cccc5c4sc4ccccc45)n3)c2)cc1 ; c1ccc(-c2cccc(-c3ccc(-c4ccccc4)c(-c4nc(-c5cccc6c5sc5ccccc56)cc(-c5cccc6c5sc5ccccc56)n4)c3)c2)cc1 ; c1ccc(-c2cc3ccccc3c3ccccc23)c(-c2nc(-c3cccc4c3sc3ccccc34)cc(-c3cccc4c3sc3ccccc34)n2)c1]

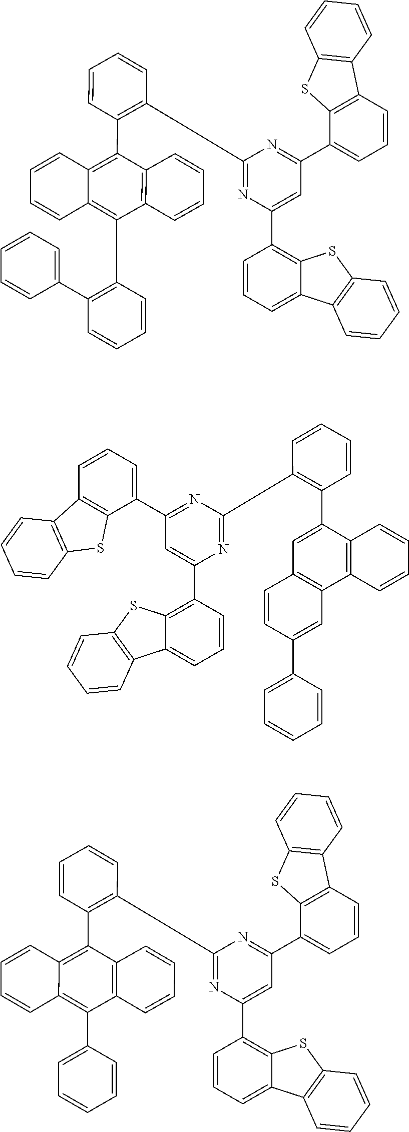 [SMILES: c1ccc(-c2ccccc2-c2c3ccccc3c(-c3ccccc3-c3nc(-c4cccc5c4sc4ccccc45)cc(-c4cccc5c4sc4ccccc45)n3)c3ccccc23)cc1 ; c1ccc(-c2ccc3cc(-c4ccccc4-c4nc(-c5cccc6c5sc5ccccc56)cc(-c5cccc6c5sc5ccccc56)n4)c4ccccc4c3c2)cc1 ; c1ccc(-c2c3ccccc3c(-c3ccccc3-c3nc(-c4cccc5c4sc4ccccc45)cc(-c4cccc5c4sc4ccccc45)n3)c3ccccc23)cc1]

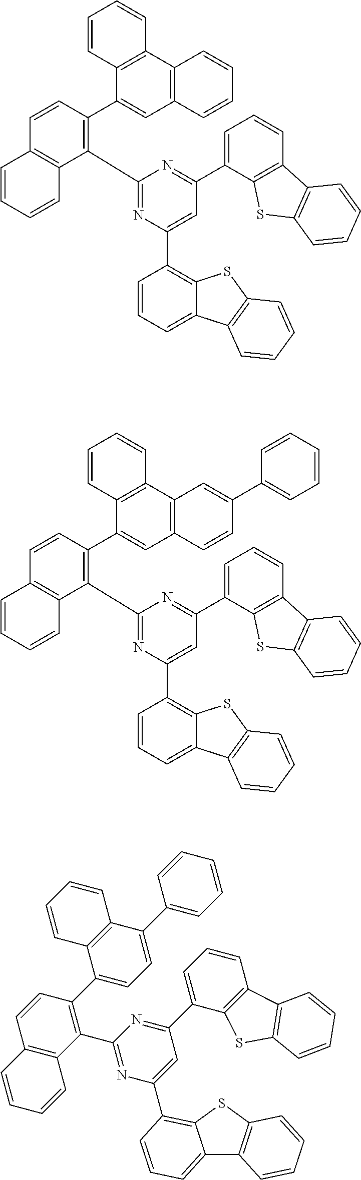 [SMILES: c1ccc2c(-c3nc(-c4cccc5c4sc4ccccc45)cc(-c4cccc5c4sc4ccccc45)n3)c(-c3cc4ccccc4c4ccccc34)ccc2c1 ; c1ccc(-c2ccc3cc(-c4ccc5ccccc5c4-c4nc(-c5cccc6c5sc5ccccc56)cc(-c5cccc6c5sc5ccccc56)n4)c4ccccc4c3c2)cc1 ; c1ccc(-c2ccc(-c3ccc4ccccc4c3-c3nc(-c4cccc5c4sc4ccccc45)cc(-c4cccc5c4sc4ccccc45)n3)c3ccccc23)cc1]

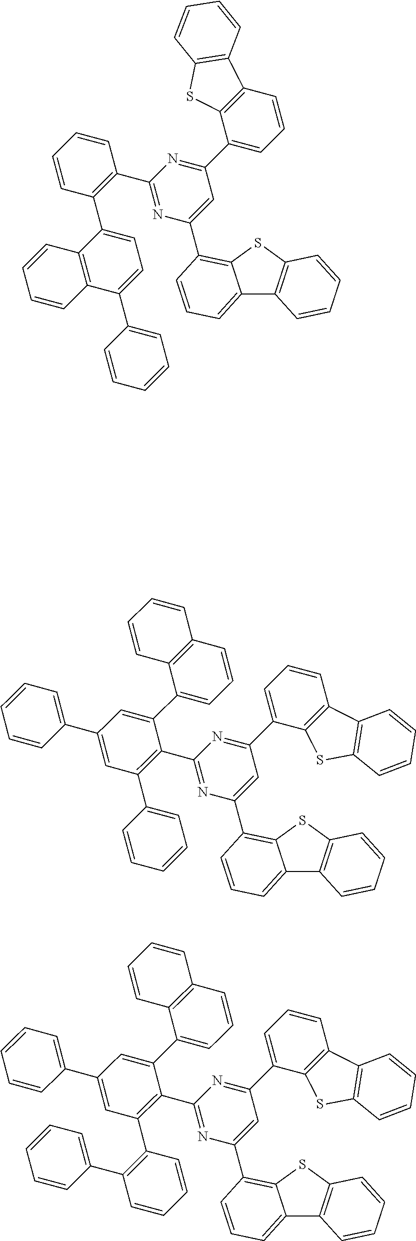 [SMILES: c1ccc(-c2ccc(-c3ccccc3-c3nc(-c4cccc5c4sc4ccccc45)cc(-c4cccc5c4sc4ccccc45)n3)c3ccccc23)cc1 ; c1ccc(-c2cc(-c3ccccc3)c(-c3nc(-c4cccc5c4sc4ccccc45)cc(-c4cccc5c4sc4ccccc45)n3)c(-c3cccc4ccccc34)c2)cc1 ; c1ccc(-c2cc(-c3ccccc3-c3ccccc3)c(-c3nc(-c4cccc5c4sc4ccccc45)cc(-c4cccc5c4sc4ccccc45)n3)c(-c3cccc4ccccc34)c2)cc1]

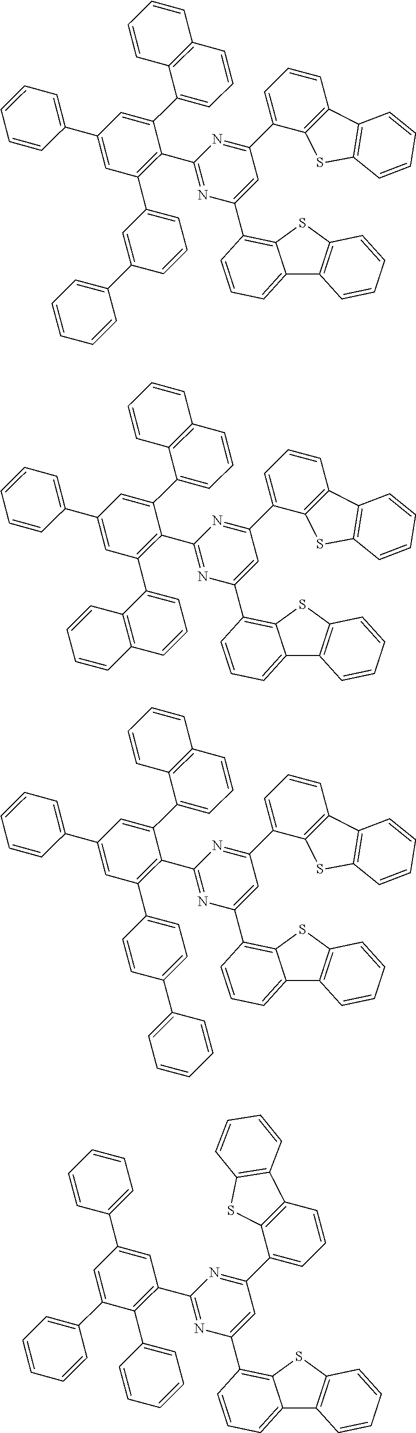 [SMILES: c1ccc(-c2cccc(-c3cc(-c4ccccc4)cc(-c4cccc5ccccc45)c3-c3nc(-c4cccc5c4sc4ccccc45)cc(-c4cccc5c4sc4ccccc45)n3)c2)cc1 ; c1ccc(-c2cc(-c3cccc4ccccc34)c(-c3nc(-c4cccc5c4sc4ccccc45)cc(-c4cccc5c4sc4ccccc45)n3)c(-c3cccc4ccccc34)c2)cc1 ; c1ccc(-c2ccc(-c3cc(-c4ccccc4)cc(-c4cccc5ccccc45)c3-c3nc(-c4cccc5c4sc4ccccc45)cc(-c4cccc5c4sc4ccccc45)n3)cc2)cc1 ; c1ccc(-c2cc(-c3ccccc3)c(-c3ccccc3)c(-c3nc(-c4cccc5c4sc4ccccc45)cc(-c4cccc5c4sc4ccccc45)n3)c2)cc1]

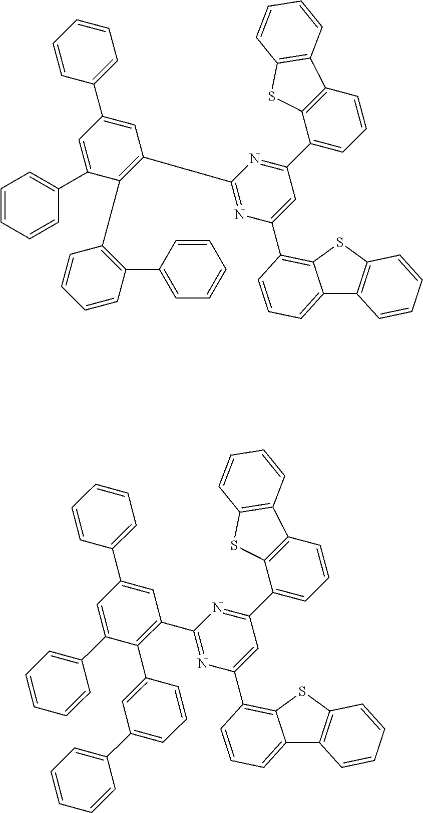 [SMILES: c1ccc(-c2cc(-c3ccccc3)c(-c3ccccc3-c3ccccc3)c(-c3nc(-c4cccc5c4sc4ccccc45)cc(-c4cccc5c4sc4ccccc45)n3)c2)cc1 ; c1ccc(-c2cccc(-c3c(-c4ccccc4)cc(-c4ccccc4)cc3-c3nc(-c4cccc5c4sc4ccccc45)cc(-c4cccc5c4sc4ccccc45)n3)c2)cc1]

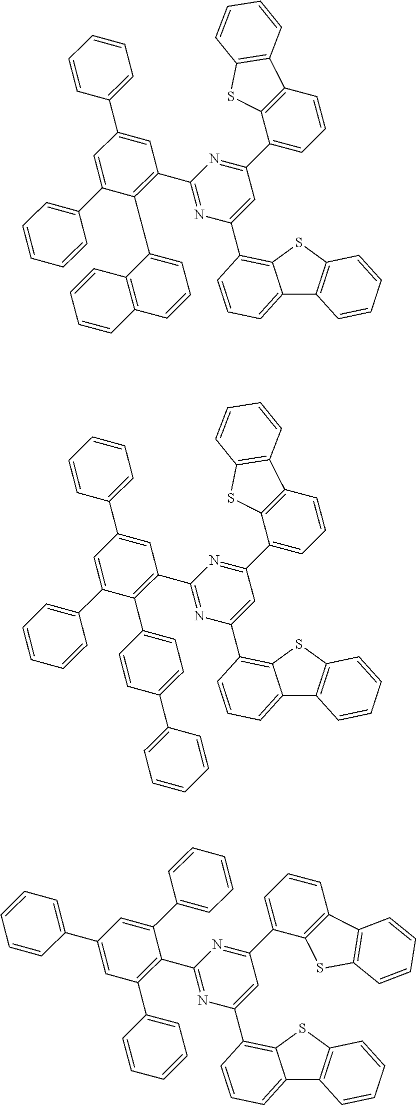 [SMILES: c1ccc(-c2cc(-c3ccccc3)c(-c3cccc4ccccc34)c(-c3nc(-c4cccc5c4sc4ccccc45)cc(-c4cccc5c4sc4ccccc45)n3)c2)cc1 ; c1ccc(-c2ccc(-c3c(-c4ccccc4)cc(-c4ccccc4)cc3-c3nc(-c4cccc5c4sc4ccccc45)cc(-c4cccc5c4sc4ccccc45)n3)cc2)cc1 ; c1ccc(-c2cc(-c3ccccc3)c(-c3nc(-c4cccc5c4sc4ccccc45)cc(-c4cccc5c4sc4ccccc45)n3)c(-c3ccccc3)c2)cc1]

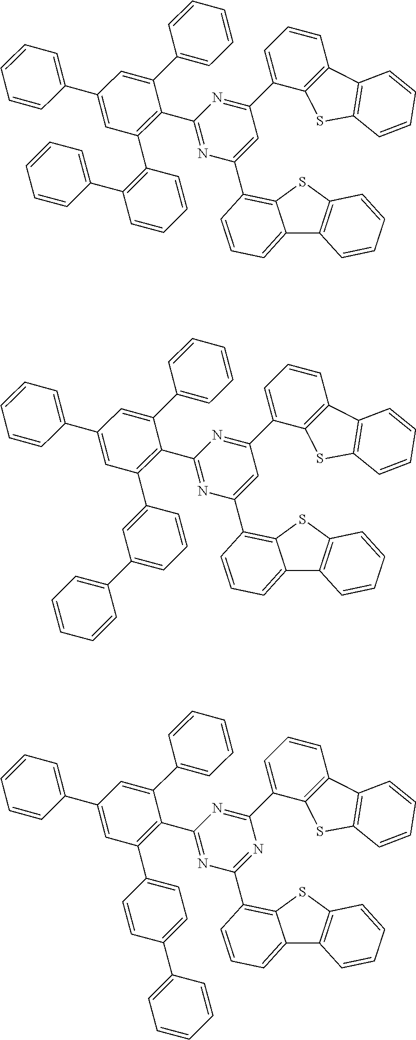 [SMILES: c1ccc(-c2cc(-c3ccccc3)c(-c3nc(-c4cccc5c4sc4ccccc45)cc(-c4cccc5c4sc4ccccc45)n3)c(-c3ccccc3-c3ccccc3)c2)cc1 ; c1ccc(-c2cccc(-c3cc(-c4ccccc4)cc(-c4ccccc4)c3-c3nc(-c4cccc5c4sc4ccccc45)cc(-c4cccc5c4sc4ccccc45)n3)c2)cc1 ; c1ccc(-c2ccc(-c3cc(-c4ccccc4)cc(-c4ccccc4)c3-c3nc(-c4cccc5c4sc4ccccc45)nc(-c4cccc5c4sc4ccccc45)n3)cc2)cc1]

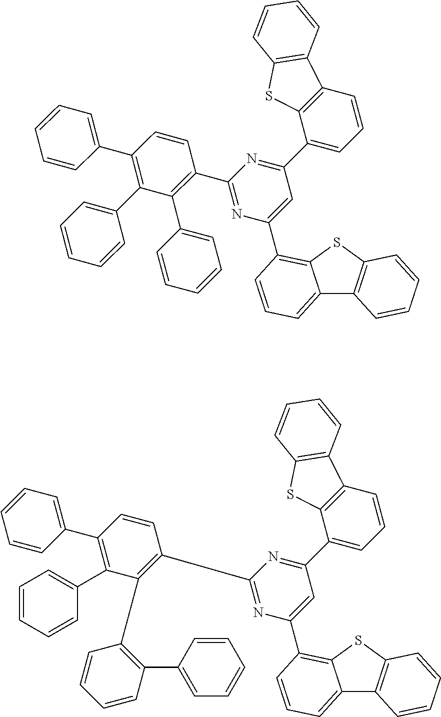 [SMILES: c1ccc(-c2ccc(-c3nc(-c4cccc5c4sc4ccccc45)cc(-c4cccc5c4sc4ccccc45)n3)c(-c3ccccc3)c2-c2ccccc2)cc1 ; c1ccc(-c2ccccc2-c2c(-c3nc(-c4cccc5c4sc4ccccc45)cc(-c4cccc5c4sc4ccccc45)n3)ccc(-c3ccccc3)c2-c2ccccc2)cc1]

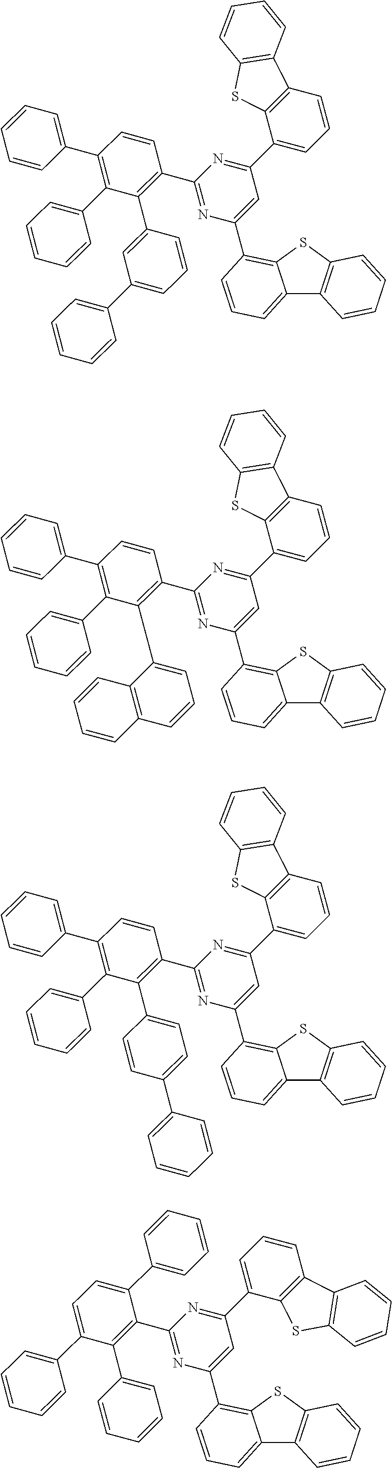

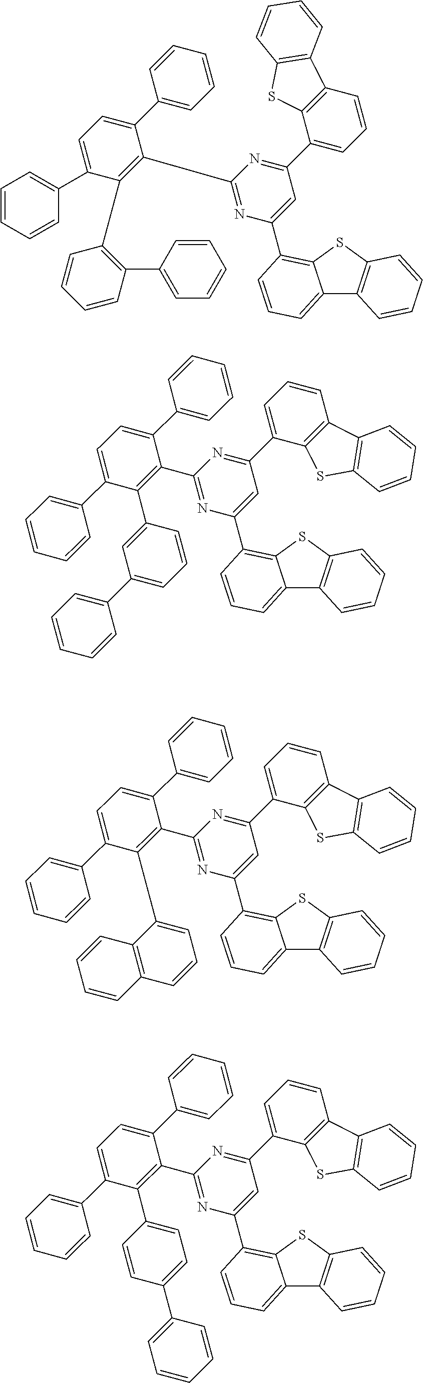 [SMILES: c1ccc(-c2ccccc2-c2c(-c3ccccc3)ccc(-c3ccccc3)c2-c2nc(-c3cccc4c3sc3ccccc34)cc(-c3cccc4c3sc3ccccc34)n2)cc1 ; c1ccc(-c2cccc(-c3c(-c4ccccc4)ccc(-c4ccccc4)c3-c3nc(-c4cccc5c4sc4ccccc45)cc(-c4cccc5c4sc4ccccc45)n3)c2)cc1 ; c1ccc(-c2ccc(-c3ccccc3)c(-c3cccc4ccccc34)c2-c2nc(-c3cccc4c3sc3ccccc34)cc(-c3cccc4c3sc3ccccc34)n2)cc1 ; c1ccc(-c2ccc(-c3c(-c4ccccc4)ccc(-c4ccccc4)c3-c3nc(-c4cccc5c4sc4ccccc45)cc(-c4cccc5c4sc4ccccc45)n3)cc2)cc1]

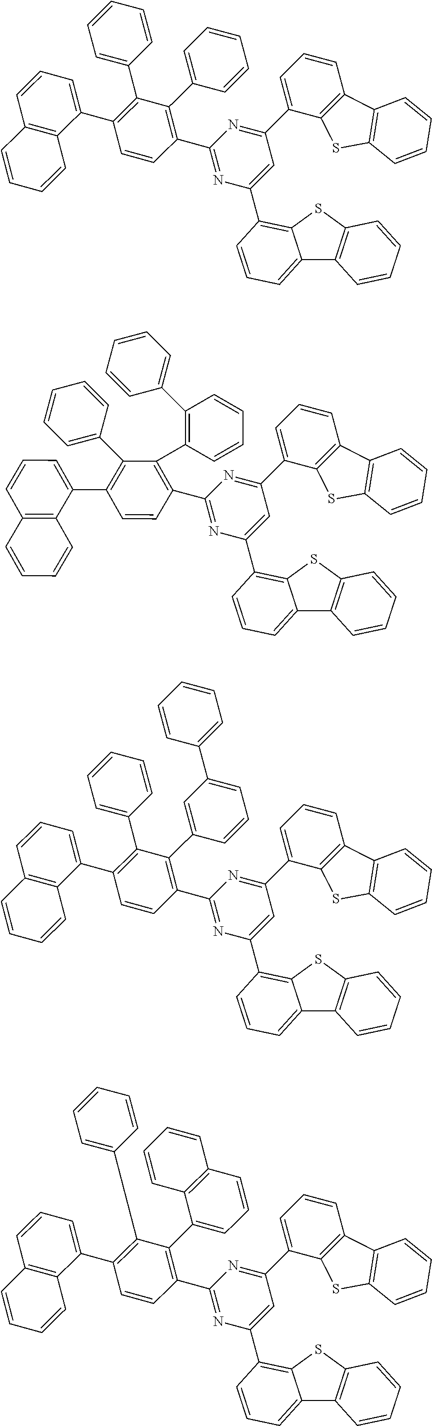 [SMILES: c1ccc(-c2c(-c3nc(-c4cccc5c4sc4ccccc45)cc(-c4cccc5c4sc4ccccc45)n3)ccc(-c3cccc4ccccc34)c2-c2ccccc2)cc1 ; c1ccc(-c2ccccc2-c2c(-c3nc(-c4cccc5c4sc4ccccc45)cc(-c4cccc5c4sc4ccccc45)n3)ccc(-c3cccc4ccccc34)c2-c2ccccc2)cc1 ; c1ccc(-c2cccc(-c3c(-c4nc(-c5cccc6c5sc5ccccc56)cc(-c5cccc6c5sc5ccccc56)n4)ccc(-c4cccc5ccccc45)c3-c3ccccc3)c2)cc1 ; c1ccc(-c2c(-c3cccc4ccccc34)ccc(-c3nc(-c4cccc5c4sc4ccccc45)cc(-c4cccc5c4sc4ccccc45)n3)c2-c2cccc3ccccc23)cc1]

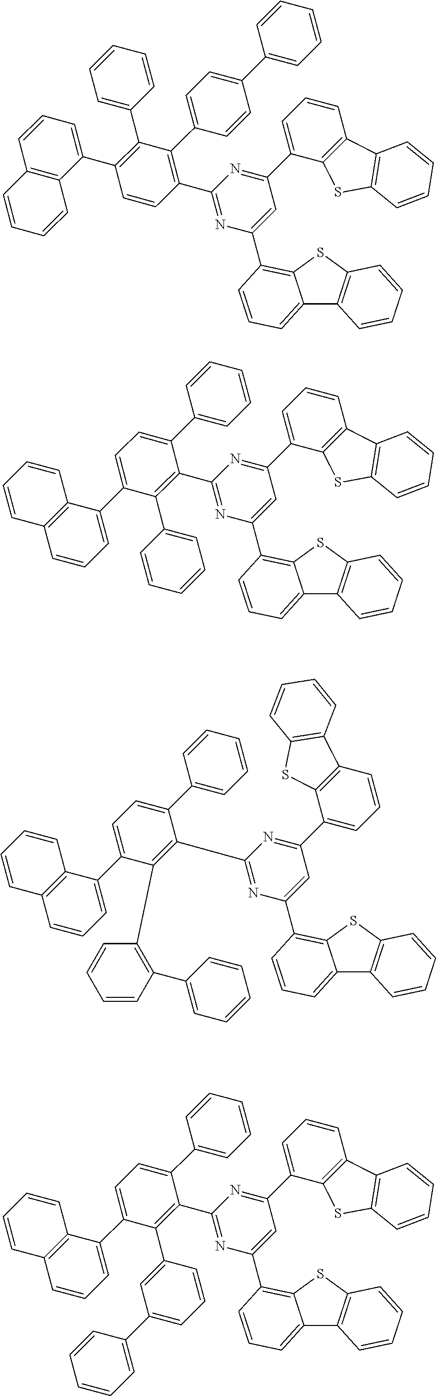 [SMILES: c1ccc(-c2ccc(-c3c(-c4nc(-c5cccc6c5sc5ccccc56)cc(-c5cccc6c5sc5ccccc56)n4)ccc(-c4cccc5ccccc45)c3-c3ccccc3)cc2)cc1 ; c1ccc(-c2ccc(-c3cccc4ccccc34)c(-c3ccccc3)c2-c2nc(-c3cccc4c3sc3ccccc34)cc(-c3cccc4c3sc3ccccc34)n2)cc1 ; c1ccc(-c2ccccc2-c2c(-c3cccc4ccccc34)ccc(-c3ccccc3)c2-c2nc(-c3cccc4c3sc3ccccc34)cc(-c3cccc4c3sc3ccccc34)n2)cc1 ; c1ccc(-c2cccc(-c3c(-c4cccc5ccccc45)ccc(-c4ccccc4)c3-c3nc(-c4cccc5c4sc4ccccc45)cc(-c4cccc5c4sc4ccccc45)n3)c2)cc1]

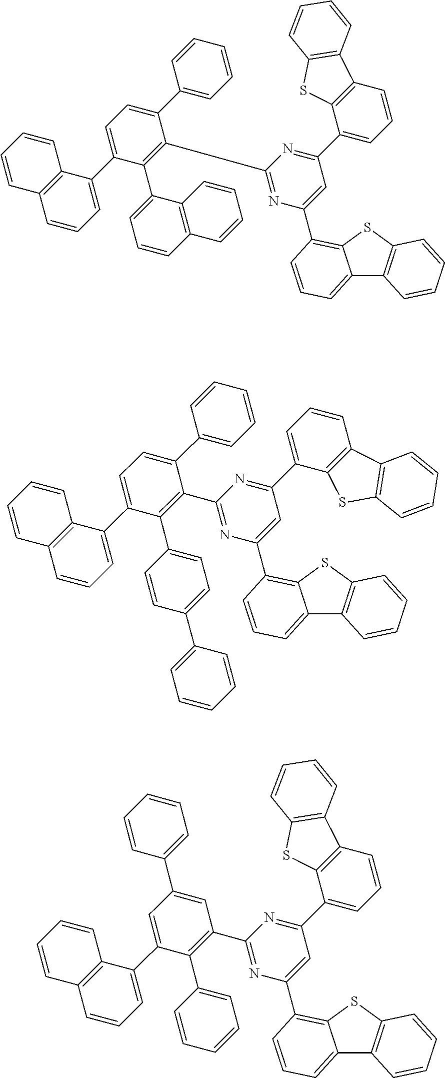 [SMILES: c1ccc(-c2ccc(-c3cccc4ccccc34)c(-c3cccc4ccccc34)c2-c2nc(-c3cccc4c3sc3ccccc34)cc(-c3cccc4c3sc3ccccc34)n2)cc1 ; c1ccc(-c2ccc(-c3c(-c4cccc5ccccc45)ccc(-c4ccccc4)c3-c3nc(-c4cccc5c4sc4ccccc45)cc(-c4cccc5c4sc4ccccc45)n3)cc2)cc1 ; c1ccc(-c2cc(-c3nc(-c4cccc5c4sc4ccccc45)cc(-c4cccc5c4sc4ccccc45)n3)c(-c3ccccc3)c(-c3cccc4ccccc34)c2)cc1]

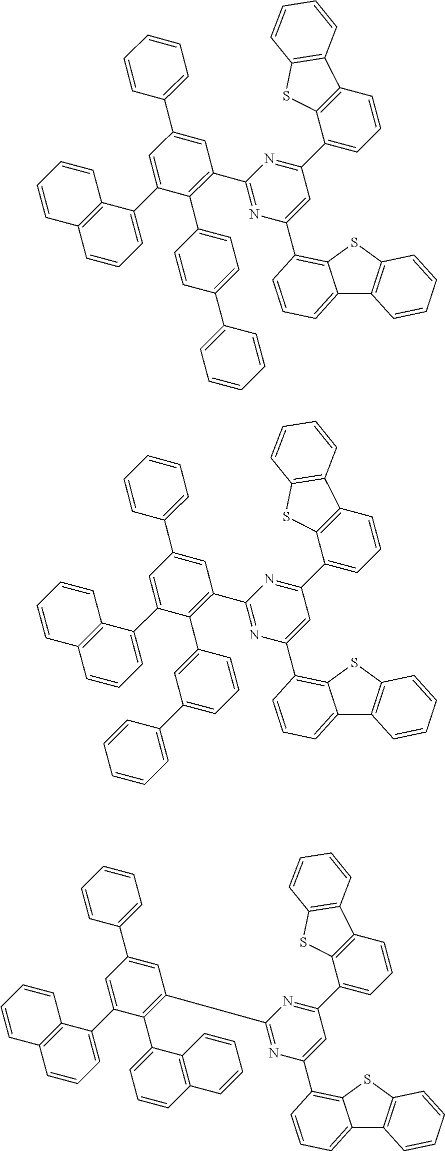 [SMILES: c1ccc(-c2ccc(-c3c(-c4nc(-c5cccc6c5sc5ccccc56)cc(-c5cccc6c5sc5ccccc56)n4)cc(-c4ccccc4)cc3-c3cccc4ccccc34)cc2)cc1 ; c1ccc(-c2cccc(-c3c(-c4nc(-c5cccc6c5sc5ccccc56)cc(-c5cccc6c5sc5ccccc56)n4)cc(-c4ccccc4)cc3-c3cccc4ccccc34)c2)cc1 ; c1ccc(-c2cc(-c3nc(-c4cccc5c4sc4ccccc45)cc(-c4cccc5c4sc4ccccc45)n3)c(-c3cccc4ccccc34)c(-c3cccc4ccccc34)c2)cc1]

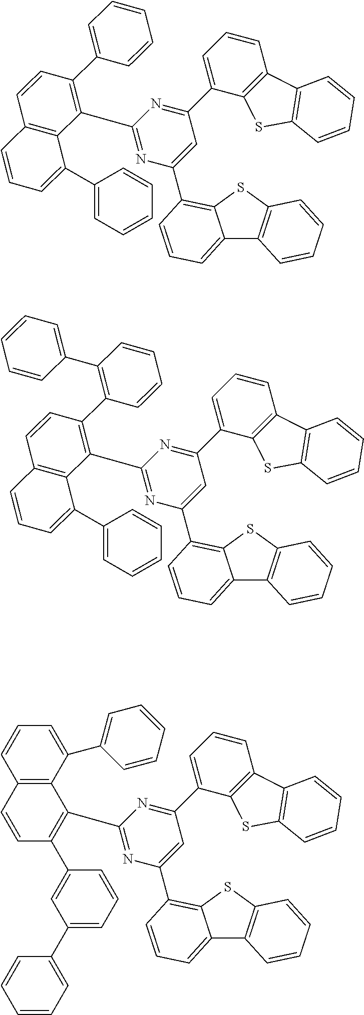 [SMILES: c1ccc(-c2ccc3cccc(-c4ccccc4)c3c2-c2nc(-c3cccc4c3sc3ccccc34)cc(-c3cccc4c3sc3ccccc34)n2)cc1 ; c1ccc(-c2ccccc2-c2ccc3cccc(-c4ccccc4)c3c2-c2nc(-c3cccc4c3sc3ccccc34)cc(-c3cccc4c3sc3ccccc34)n2)cc1 ; c1ccc(-c2cccc(-c3ccc4cccc(-c5ccccc5)c4c3-c3nc(-c4cccc5c4sc4ccccc45)cc(-c4cccc5c4sc4ccccc45)n3)c2)cc1]

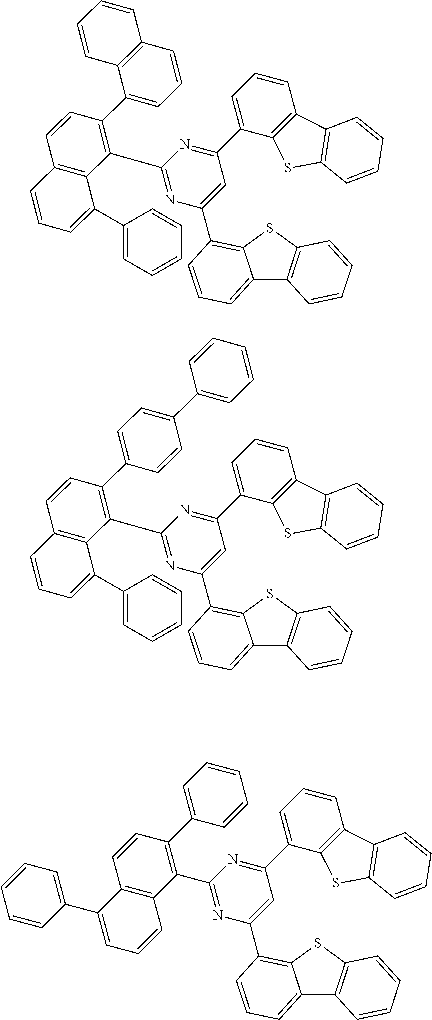

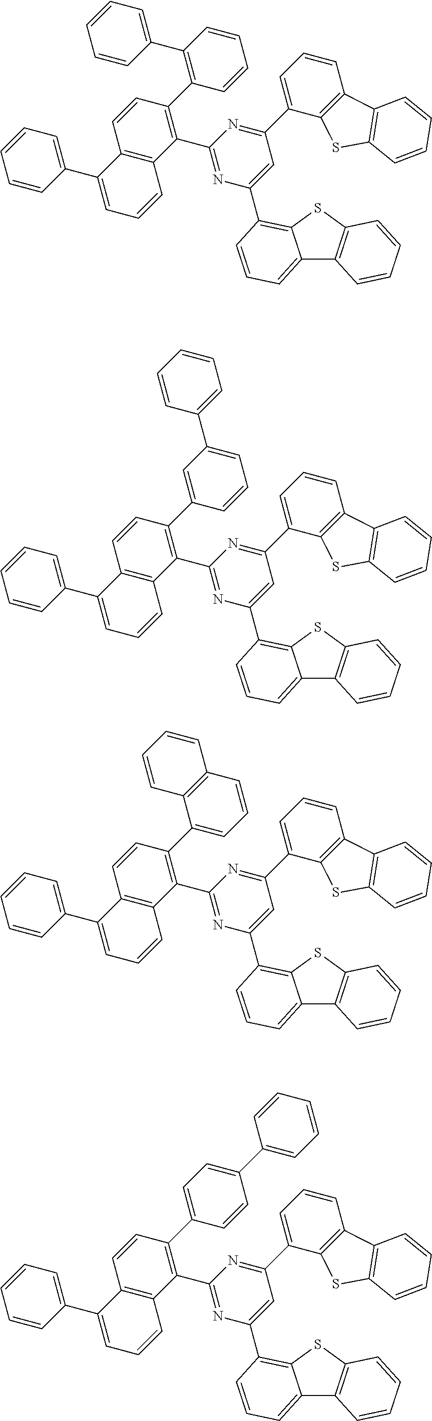 [SMILES: c1ccc(-c2ccccc2-c2ccc3c(-c4ccccc4)cccc3c2-c2nc(-c3cccc4c3sc3ccccc34)cc(-c3cccc4c3sc3ccccc34)n2)cc1 ; c1ccc(-c2cccc(-c3ccc4c(-c5ccccc5)cccc4c3-c3nc(-c4cccc5c4sc4ccccc45)cc(-c4cccc5c4sc4ccccc45)n3)c2)cc1 ; c1ccc(-c2cccc3c(-c4nc(-c5cccc6c5sc5ccccc56)cc(-c5cccc6c5sc5ccccc56)n4)c(-c4cccc5ccccc45)ccc23)cc1 ; c1ccc(-c2ccc(-c3ccc4c(-c5ccccc5)cccc4c3-c3nc(-c4cccc5c4sc4ccccc45)cc(-c4cccc5c4sc4ccccc45)n3)cc2)cc1]

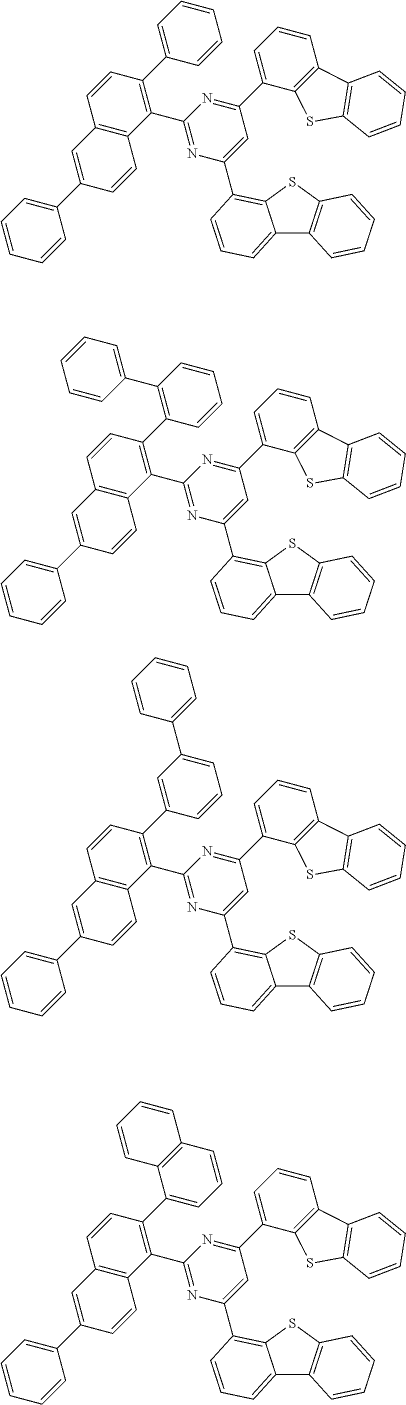 [SMILES: c1ccc(-c2ccc3c(-c4nc(-c5cccc6c5sc5ccccc56)cc(-c5cccc6c5sc5ccccc56)n4)c(-c4ccccc4)ccc3c2)cc1 ; c1ccc(-c2ccc3c(-c4nc(-c5cccc6c5sc5ccccc56)cc(-c5cccc6c5sc5ccccc56)n4)c(-c4ccccc4-c4ccccc4)ccc3c2)cc1 ; c1ccc(-c2cccc(-c3ccc4cc(-c5ccccc5)ccc4c3-c3nc(-c4cccc5c4sc4ccccc45)cc(-c4cccc5c4sc4ccccc45)n3)c2)cc1 ; c1ccc(-c2ccc3c(-c4nc(-c5cccc6c5sc5ccccc56)cc(-c5cccc6c5sc5ccccc56)n4)c(-c4cccc5ccccc45)ccc3c2)cc1]

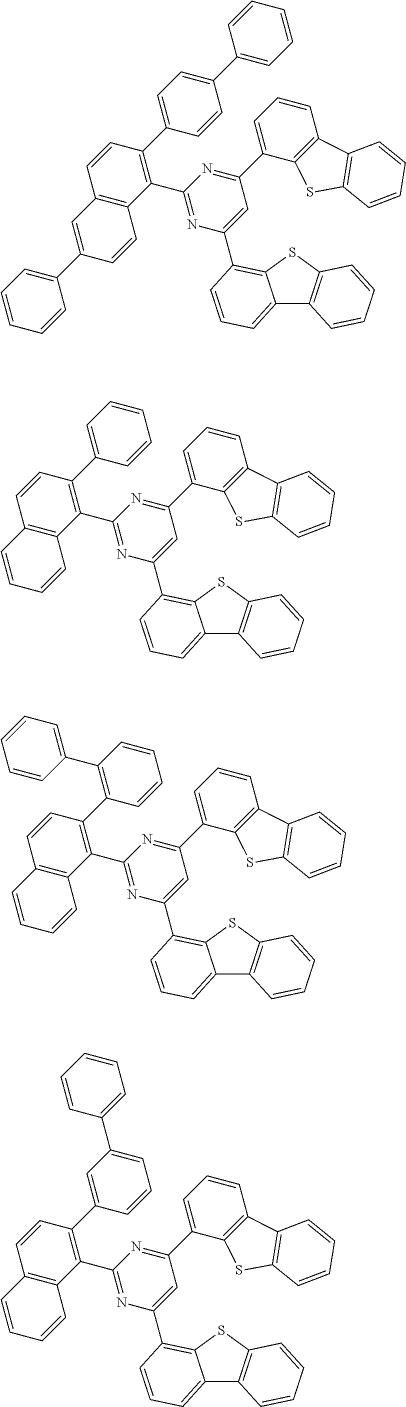 [SMILES: c1ccc(-c2ccc(-c3ccc4cc(-c5ccccc5)ccc4c3-c3nc(-c4cccc5c4sc4ccccc45)cc(-c4cccc5c4sc4ccccc45)n3)cc2)cc1 ; c1ccc(-c2ccc3ccccc3c2-c2nc(-c3cccc4c3sc3ccccc34)cc(-c3cccc4c3sc3ccccc34)n2)cc1 ; c1ccc(-c2ccccc2-c2ccc3ccccc3c2-c2nc(-c3cccc4c3sc3ccccc34)cc(-c3cccc4c3sc3ccccc34)n2)cc1 ; c1ccc(-c2cccc(-c3ccc4ccccc4c3-c3nc(-c4cccc5c4sc4ccccc45)cc(-c4cccc5c4sc4ccccc45)n3)c2)cc1]

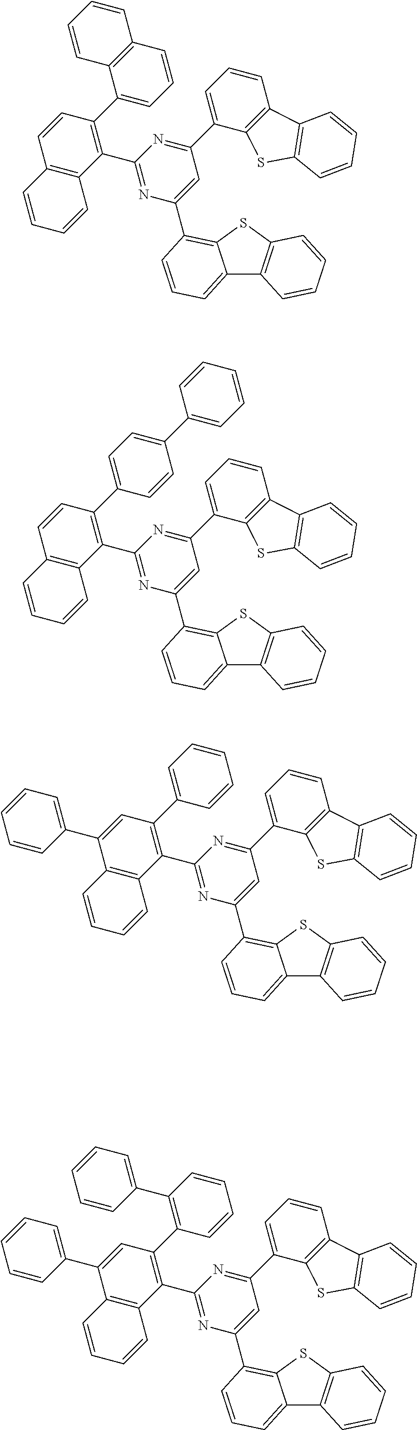 [SMILES: c1ccc2c(-c3ccc4ccccc4c3-c3nc(-c4cccc5c4sc4ccccc45)cc(-c4cccc5c4sc4ccccc45)n3)cccc2c1 ; c1ccc(-c2ccc(-c3ccc4ccccc4c3-c3nc(-c4cccc5c4sc4ccccc45)cc(-c4cccc5c4sc4ccccc45)n3)cc2)cc1 ; c1ccc(-c2cc(-c3ccccc3)c3ccccc3c2-c2nc(-c3cccc4c3sc3ccccc34)cc(-c3cccc4c3sc3ccccc34)n2)cc1 ; c1ccc(-c2ccccc2-c2cc(-c3ccccc3)c3ccccc3c2-c2nc(-c3cccc4c3sc3ccccc34)cc(-c3cccc4c3sc3ccccc34)n2)cc1]

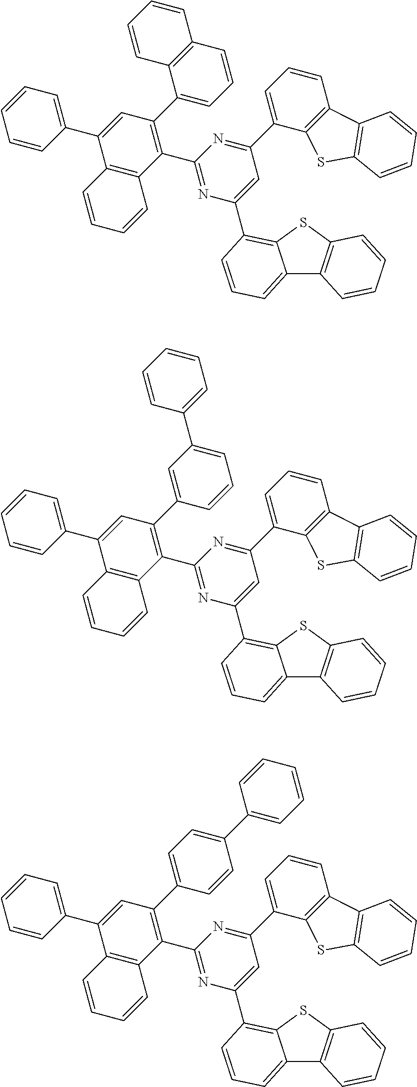 [SMILES: c1ccc(-c2cc(-c3cccc4ccccc34)c(-c3nc(-c4cccc5c4sc4ccccc45)cc(-c4cccc5c4sc4ccccc45)n3)c3ccccc23)cc1 ; c1ccc(-c2cccc(-c3cc(-c4ccccc4)c4ccccc4c3-c3nc(-c4cccc5c4sc4ccccc45)cc(-c4cccc5c4sc4ccccc45)n3)c2)cc1 ; c1ccc(-c2ccc(-c3cc(-c4ccccc4)c4ccccc4c3-c3nc(-c4cccc5c4sc4ccccc45)cc(-c4cccc5c4sc4ccccc45)n3)cc2)cc1]

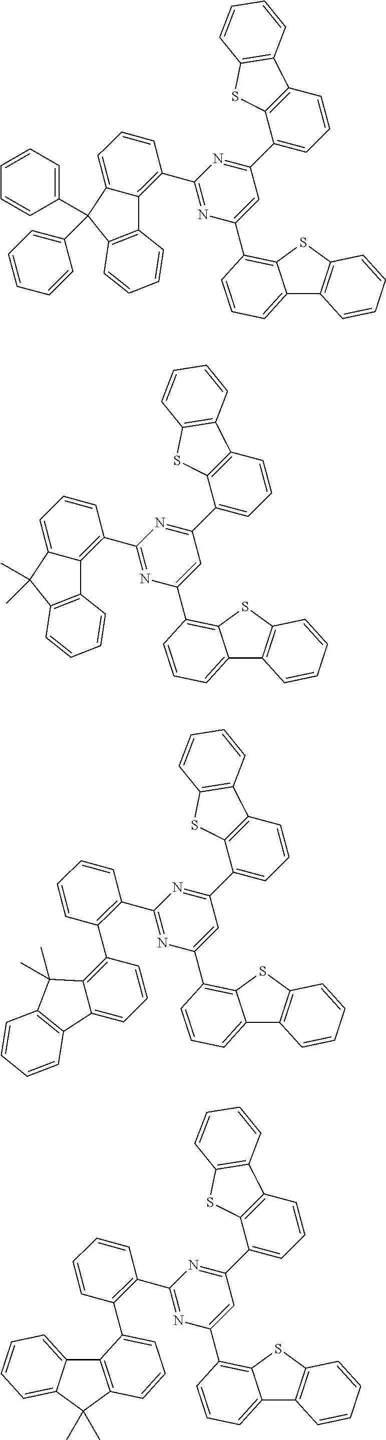 [SMILES: c1ccc(C2(c3ccccc3)c3ccccc3-c3c(-c4nc(-c5cccc6c5sc5ccccc56)cc(-c5cccc6c5sc5ccccc56)n4)cccc32)cc1 ; CC1(C)c2ccccc2-c2c(-c3nc(-c4cccc5c4sc4ccccc45)cc(-c4cccc5c4sc4ccccc45)n3)cccc21 ; CC1(C)c2ccccc2-c2cccc(-c3ccccc3-c3nc(-c4cccc5c4sc4ccccc45)cc(-c4cccc5c4sc4ccccc45)n3)c21 ; CC1(C)c2ccccc2-c2c(-c3ccccc3-c3nc(-c4cccc5c4sc4ccccc45)cc(-c4cccc5c4sc4ccccc45)n3)cccc21]

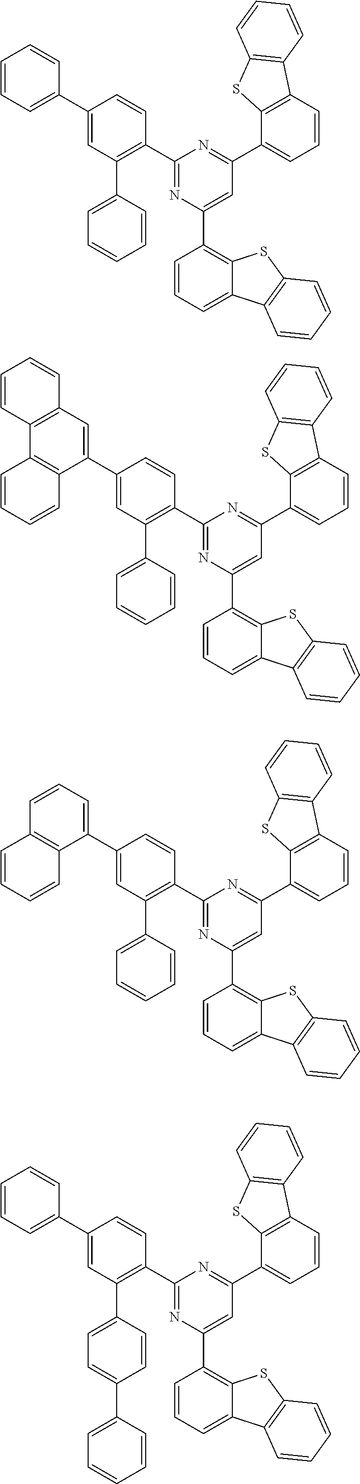

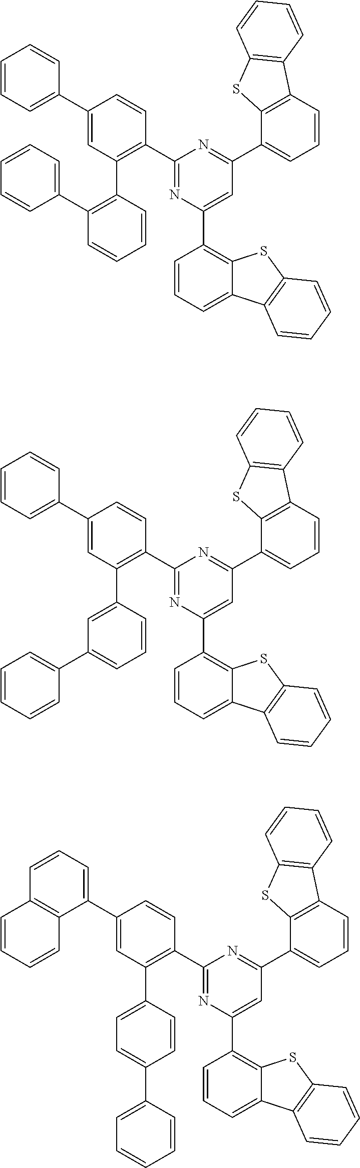 [SMILES: c1ccc(-c2ccc(-c3nc(-c4cccc5c4sc4ccccc45)cc(-c4cccc5c4sc4ccccc45)n3)c(-c3ccccc3-c3ccccc3)c2)cc1 ; c1ccc(-c2cccc(-c3cc(-c4ccccc4)ccc3-c3nc(-c4cccc5c4sc4ccccc45)cc(-c4cccc5c4sc4ccccc45)n3)c2)cc1 ; c1ccc(-c2ccc(-c3cc(-c4cccc5ccccc45)ccc3-c3nc(-c4cccc5c4sc4ccccc45)cc(-c4cccc5c4sc4ccccc45)n3)cc2)cc1]

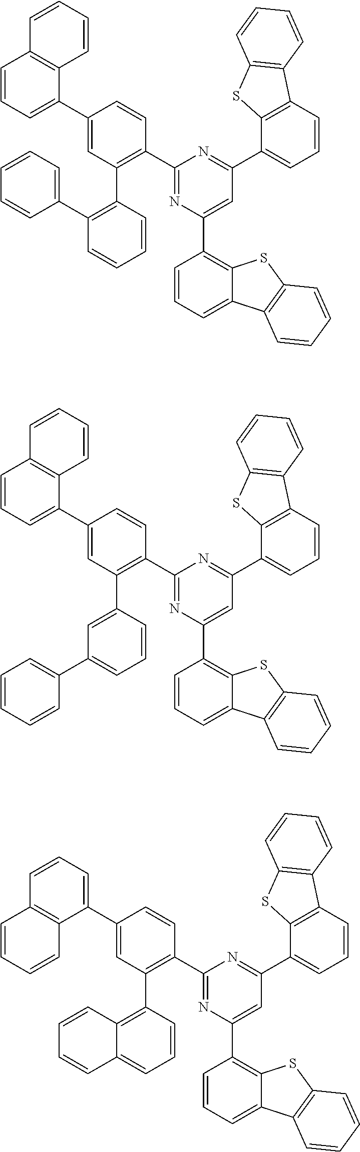 [SMILES: c1ccc(-c2ccccc2-c2cc(-c3cccc4ccccc34)ccc2-c2nc(-c3cccc4c3sc3ccccc34)cc(-c3cccc4c3sc3ccccc34)n2)cc1 ; c1ccc(-c2cccc(-c3cc(-c4cccc5ccccc45)ccc3-c3nc(-c4cccc5c4sc4ccccc45)cc(-c4cccc5c4sc4ccccc45)n3)c2)cc1 ; c1ccc2c(-c3ccc(-c4nc(-c5cccc6c5sc5ccccc56)cc(-c5cccc6c5sc5ccccc56)n4)c(-c4cccc5ccccc45)c3)cccc2c1]

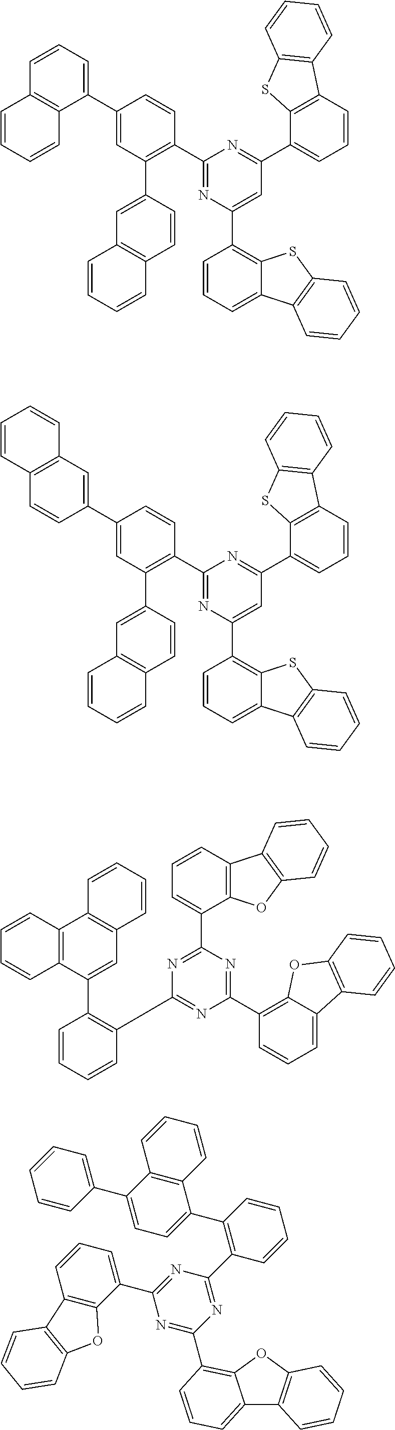 [SMILES: c1ccc2cc(-c3cc(-c4cccc5ccccc45)ccc3-c3nc(-c4cccc5c4sc4ccccc45)cc(-c4cccc5c4sc4ccccc45)n3)ccc2c1 ; c1ccc2cc(-c3ccc(-c4nc(-c5cccc6c5sc5ccccc56)cc(-c5cccc6c5sc5ccccc56)n4)c(-c4ccc5ccccc5c4)c3)ccc2c1 ; c1ccc(-c2cc3ccccc3c3ccccc23)c(-c2nc(-c3cccc4c3oc3ccccc34)nc(-c3cccc4c3oc3ccccc34)n2)c1 ; c1ccc(-c2ccc(-c3ccccc3-c3nc(-c4cccc5c4oc4ccccc45)nc(-c4cccc5c4oc4ccccc45)n3)c3ccccc23)cc1]

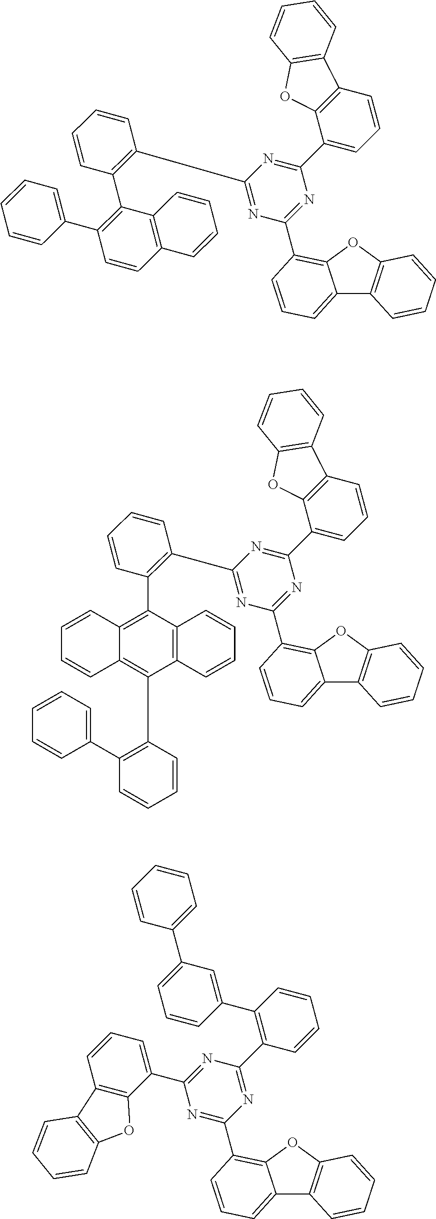 [SMILES: c1ccc(-c2ccc3ccccc3c2-c2ccccc2-c2nc(-c3cccc4c3oc3ccccc34)nc(-c3cccc4c3oc3ccccc34)n2)cc1 ; c1ccc(-c2ccccc2-c2c3ccccc3c(-c3ccccc3-c3nc(-c4cccc5c4oc4ccccc45)nc(-c4cccc5c4oc4ccccc45)n3)c3ccccc23)cc1 ; c1ccc(-c2cccc(-c3ccccc3-c3nc(-c4cccc5c4oc4ccccc45)nc(-c4cccc5c4oc4ccccc45)n3)c2)cc1]

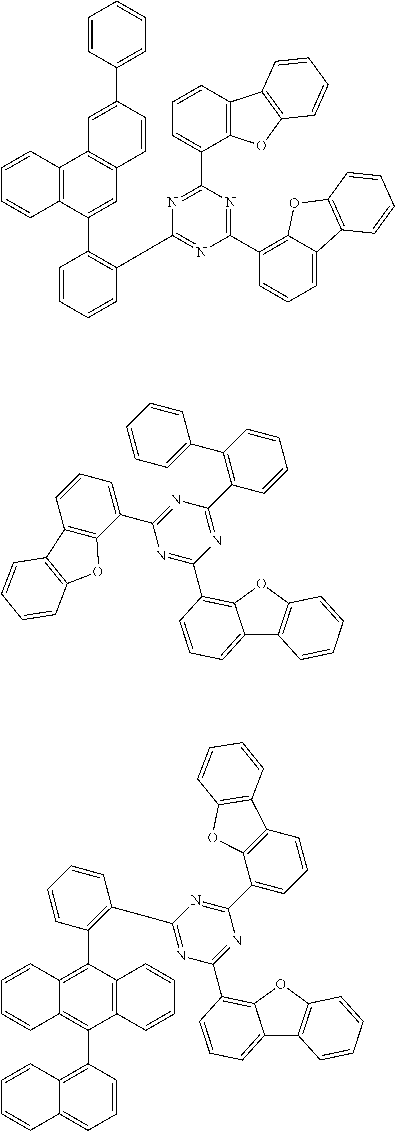 [SMILES: c1ccc(-c2ccc3cc(-c4ccccc4-c4nc(-c5cccc6c5oc5ccccc56)nc(-c5cccc6c5oc5ccccc56)n4)c4ccccc4c3c2)cc1 ; c1ccc(-c2ccccc2-c2nc(-c3cccc4c3oc3ccccc34)nc(-c3cccc4c3oc3ccccc34)n2)cc1 ; c1ccc(-c2c3ccccc3c(-c3cccc4ccccc34)c3ccccc23)c(-c2nc(-c3cccc4c3oc3ccccc34)nc(-c3cccc4c3oc3ccccc34)n2)c1]

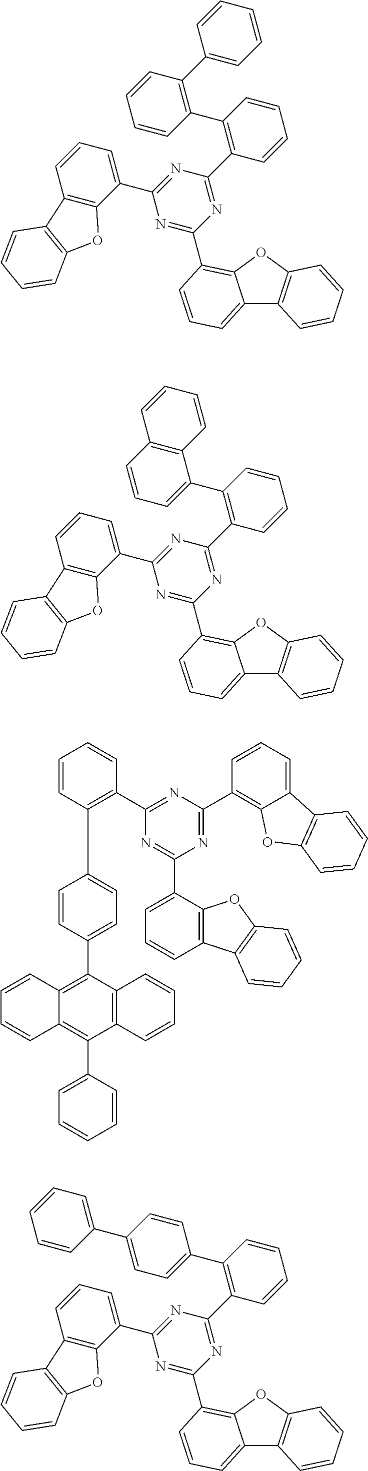 [SMILES: c1ccc(-c2ccccc2-c2ccccc2-c2nc(-c3cccc4c3oc3ccccc34)nc(-c3cccc4c3oc3ccccc34)n2)cc1 ; c1ccc(-c2cccc3ccccc23)c(-c2nc(-c3cccc4c3oc3ccccc34)nc(-c3cccc4c3oc3ccccc34)n2)c1 ; c1ccc(-c2c3ccccc3c(-c3ccc(-c4ccccc4-c4nc(-c5cccc6c5oc5ccccc56)nc(-c5cccc6c5oc5ccccc56)n4)cc3)c3ccccc23)cc1 ; c1ccc(-c2ccc(-c3ccccc3-c3nc(-c4cccc5c4oc4ccccc45)nc(-c4cccc5c4oc4ccccc45)n3)cc2)cc1]

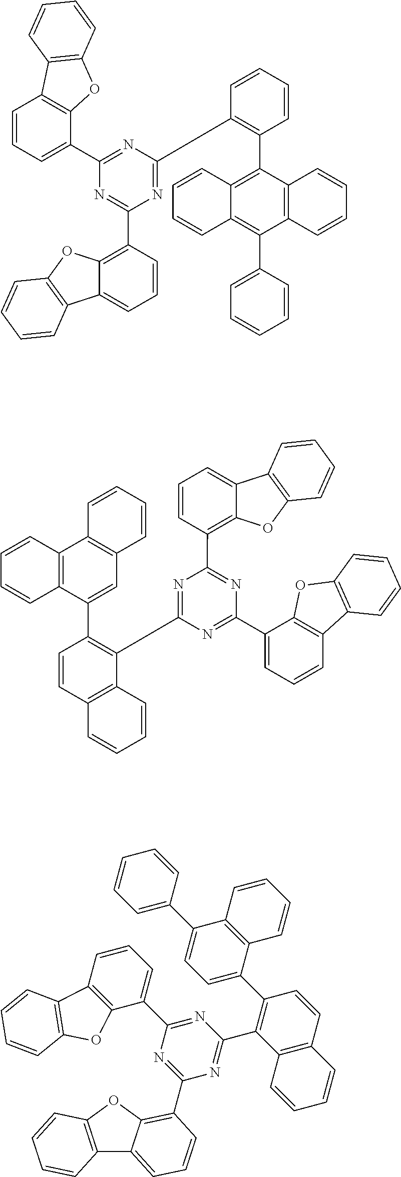 [SMILES: c1ccc(-c2c3ccccc3c(-c3ccccc3-c3nc(-c4cccc5c4oc4ccccc45)nc(-c4cccc5c4oc4ccccc45)n3)c3ccccc23)cc1 ; c1ccc2c(-c3nc(-c4cccc5c4oc4ccccc45)nc(-c4cccc5c4oc4ccccc45)n3)c(-c3cc4ccccc4c4ccccc34)ccc2c1 ; c1ccc(-c2ccc(-c3ccc4ccccc4c3-c3nc(-c4cccc5c4oc4ccccc45)nc(-c4cccc5c4oc4ccccc45)n3)c3ccccc23)cc1]

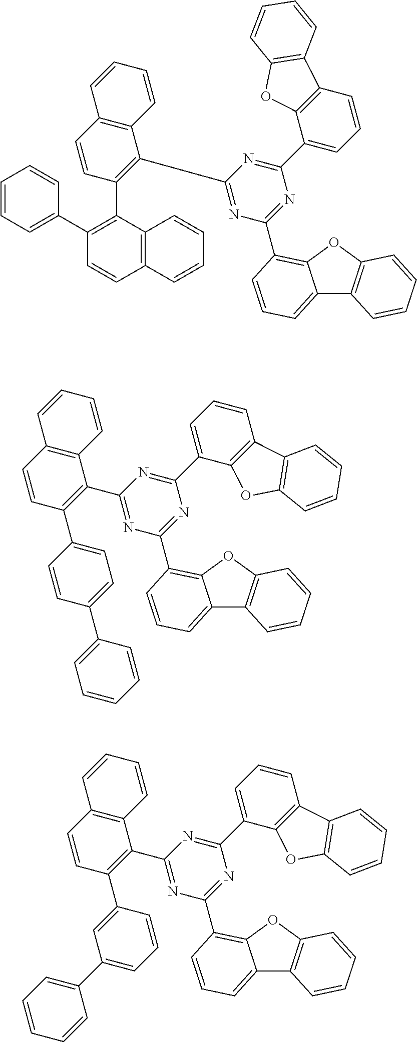 [SMILES: c1ccc(-c2ccc3ccccc3c2-c2ccc3ccccc3c2-c2nc(-c3cccc4c3oc3ccccc34)nc(-c3cccc4c3oc3ccccc34)n2)cc1 ; c1ccc(-c2ccc(-c3ccc4ccccc4c3-c3nc(-c4cccc5c4oc4ccccc45)nc(-c4cccc5c4oc4ccccc45)n3)cc2)cc1 ; c1ccc(-c2cccc(-c3ccc4ccccc4c3-c3nc(-c4cccc5c4oc4ccccc45)nc(-c4cccc5c4oc4ccccc45)n3)c2)cc1]

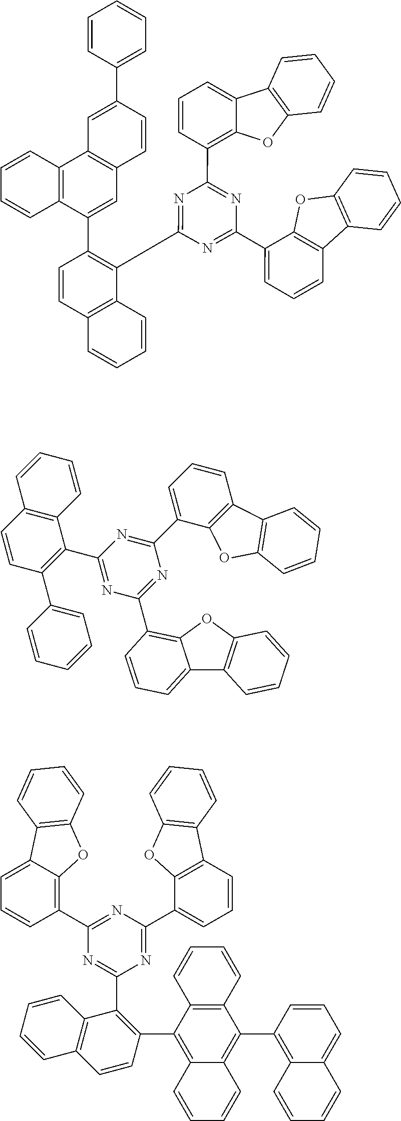 [SMILES: c1ccc(-c2ccc3cc(-c4ccc5ccccc5c4-c4nc(-c5cccc6c5oc5ccccc56)nc(-c5cccc6c5oc5ccccc56)n4)c4ccccc4c3c2)cc1 ; c1ccc(-c2ccc3ccccc3c2-c2nc(-c3cccc4c3oc3ccccc34)nc(-c3cccc4c3oc3ccccc34)n2)cc1 ; c1ccc2c(-c3c4ccccc4c(-c4ccc5ccccc5c4-c4nc(-c5cccc6c5oc5ccccc56)nc(-c5cccc6c5oc5ccccc56)n4)c4ccccc34)cccc2c1]

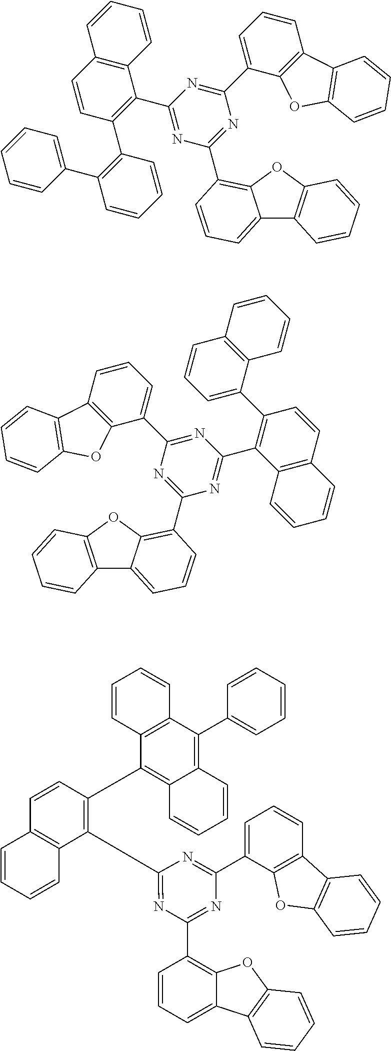 [SMILES: c1ccc(-c2ccccc2-c2ccc3ccccc3c2-c2nc(-c3cccc4c3oc3ccccc34)nc(-c3cccc4c3oc3ccccc34)n2)cc1 ; c1ccc2c(-c3ccc4ccccc4c3-c3nc(-c4cccc5c4oc4ccccc45)nc(-c4cccc5c4oc4ccccc45)n3)cccc2c1 ; c1ccc(-c2c3ccccc3c(-c3ccc4ccccc4c3-c3nc(-c4cccc5c4oc4ccccc45)nc(-c4cccc5c4oc4ccccc45)n3)c3ccccc23)cc1]

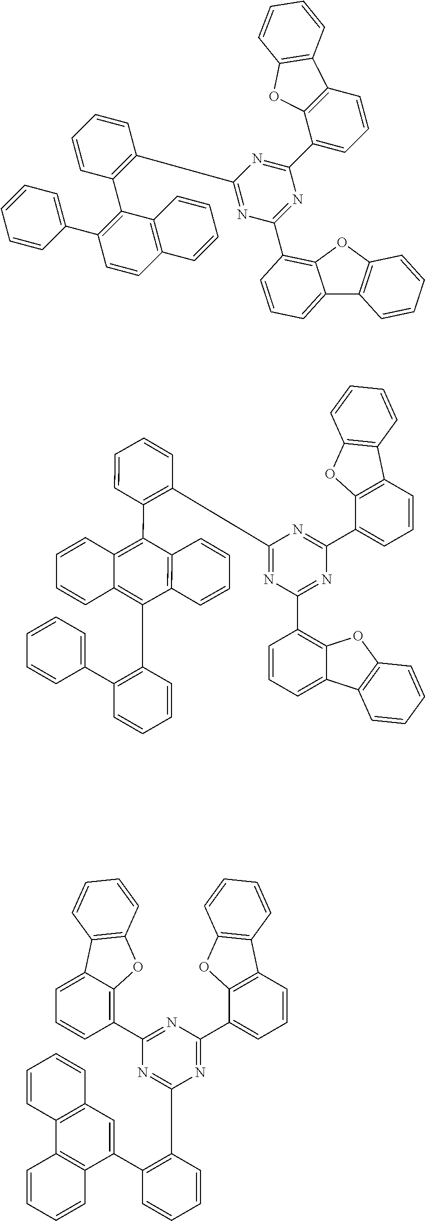 [SMILES: c1ccc(-c2ccc3ccccc3c2-c2ccccc2-c2nc(-c3cccc4c3oc3ccccc34)nc(-c3cccc4c3oc3ccccc34)n2)cc1 ; c1ccc(-c2ccccc2-c2c3ccccc3c(-c3ccccc3-c3nc(-c4cccc5c4oc4ccccc45)nc(-c4cccc5c4oc4ccccc45)n3)c3ccccc23)cc1 ; c1ccc(-c2cc3ccccc3c3ccccc23)c(-c2nc(-c3cccc4c3oc3ccccc34)nc(-c3cccc4c3oc3ccccc34)n2)c1]

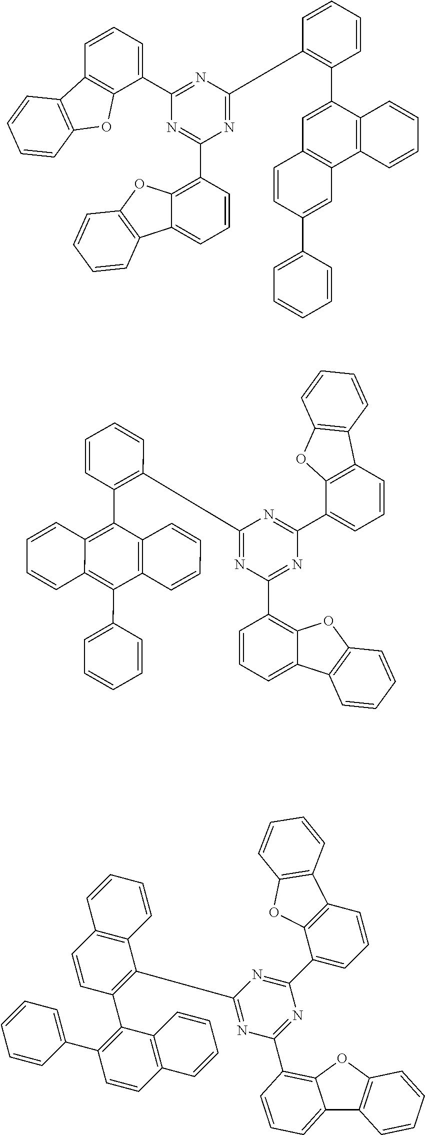 [SMILES: c1ccc(-c2ccc3cc(-c4ccccc4-c4nc(-c5cccc6c5oc5ccccc56)nc(-c5cccc6c5oc5ccccc56)n4)c4ccccc4c3c2)cc1 ; c1ccc(-c2c3ccccc3c(-c3ccccc3-c3nc(-c4cccc5c4oc4ccccc45)nc(-c4cccc5c4oc4ccccc45)n3)c3ccccc23)cc1 ; c1ccc(-c2ccc3ccccc3c2-c2ccc3ccccc3c2-c2nc(-c3cccc4c3oc3ccccc34)nc(-c3cccc4c3oc3ccccc34)n2)cc1]

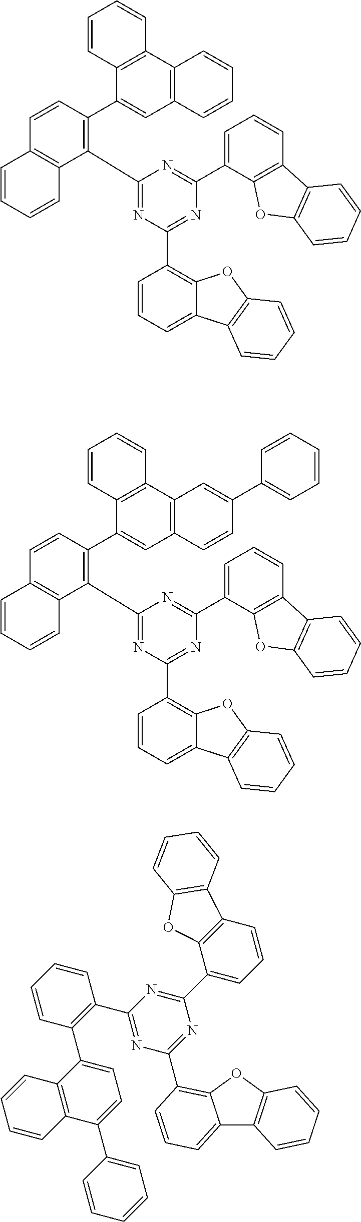 [SMILES: c1ccc2c(-c3nc(-c4cccc5c4oc4ccccc45)nc(-c4cccc5c4oc4ccccc45)n3)c(-c3cc4ccccc4c4ccccc34)ccc2c1 ; c1ccc(-c2ccc3cc(-c4ccc5ccccc5c4-c4nc(-c5cccc6c5oc5ccccc56)nc(-c5cccc6c5oc5ccccc56)n4)c4ccccc4c3c2)cc1 ; c1ccc(-c2ccc(-c3ccccc3-c3nc(-c4cccc5c4oc4ccccc45)nc(-c4cccc5c4oc4ccccc45)n3)c3ccccc23)cc1]

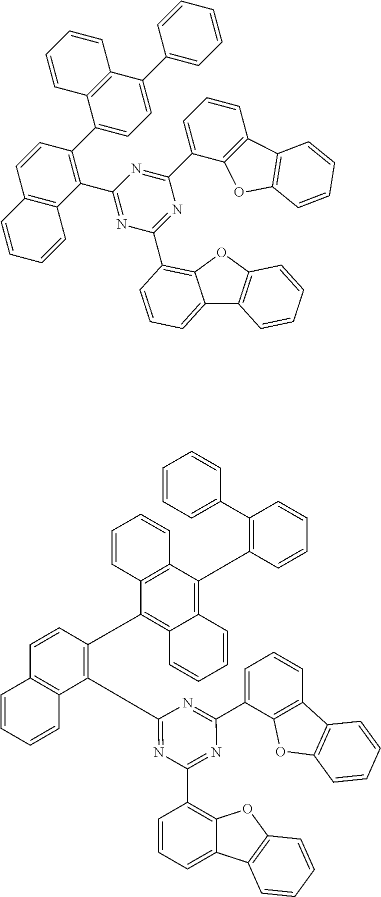 [SMILES: c1ccc(-c2ccc(-c3ccc4ccccc4c3-c3nc(-c4cccc5c4oc4ccccc45)nc(-c4cccc5c4oc4ccccc45)n3)c3ccccc23)cc1 ; c1ccc(-c2ccccc2-c2c3ccccc3c(-c3ccc4ccccc4c3-c3nc(-c4cccc5c4oc4ccccc45)nc(-c4cccc5c4oc4ccccc45)n3)c3ccccc23)cc1]

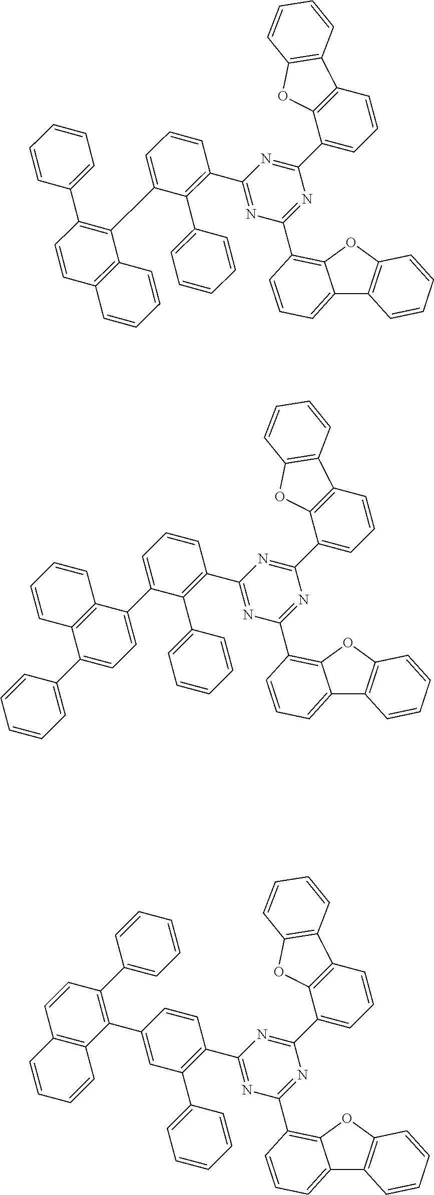 [SMILES: c1ccc(-c2ccc3ccccc3c2-c2cccc(-c3nc(-c4cccc5c4oc4ccccc45)nc(-c4cccc5c4oc4ccccc45)n3)c2-c2ccccc2)cc1 ; c1ccc(-c2c(-c3nc(-c4cccc5c4oc4ccccc45)nc(-c4cccc5c4oc4ccccc45)n3)cccc2-c2ccc(-c3ccccc3)c3ccccc23)cc1 ; c1ccc(-c2cc(-c3c(-c4ccccc4)ccc4ccccc34)ccc2-c2nc(-c3cccc4c3oc3ccccc34)nc(-c3cccc4c3oc3ccccc34)n2)cc1]

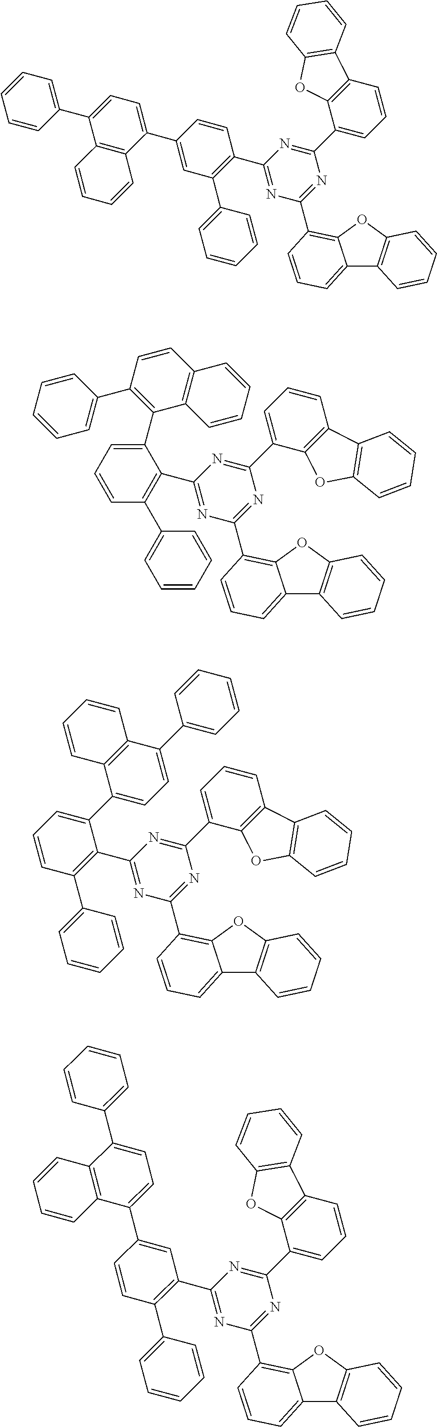 [SMILES: c1ccc(-c2cc(-c3ccc(-c4ccccc4)c4ccccc34)ccc2-c2nc(-c3cccc4c3oc3ccccc34)nc(-c3cccc4c3oc3ccccc34)n2)cc1 ; c1ccc(-c2cccc(-c3c(-c4ccccc4)ccc4ccccc34)c2-c2nc(-c3cccc4c3oc3ccccc34)nc(-c3cccc4c3oc3ccccc34)n2)cc1 ; c1ccc(-c2cccc(-c3ccc(-c4ccccc4)c4ccccc34)c2-c2nc(-c3cccc4c3oc3ccccc34)nc(-c3cccc4c3oc3ccccc34)n2)cc1 ; c1ccc(-c2ccc(-c3ccc(-c4ccccc4)c4ccccc34)cc2-c2nc(-c3cccc4c3oc3ccccc34)nc(-c3cccc4c3oc3ccccc34)n2)cc1]

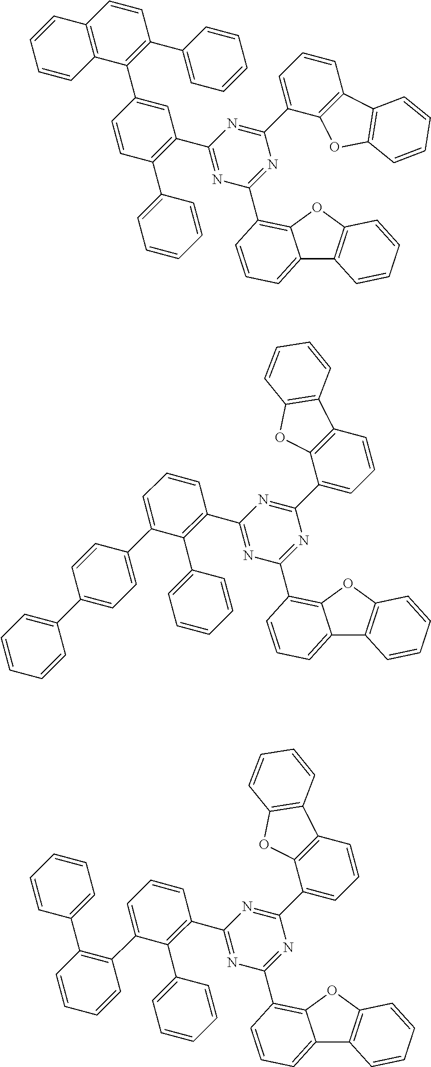 [SMILES: c1ccc(-c2ccc(-c3c(-c4ccccc4)ccc4ccccc34)cc2-c2nc(-c3cccc4c3oc3ccccc34)nc(-c3cccc4c3oc3ccccc34)n2)cc1 ; c1ccc(-c2ccc(-c3cccc(-c4nc(-c5cccc6c5oc5ccccc56)nc(-c5cccc6c5oc5ccccc56)n4)c3-c3ccccc3)cc2)cc1 ; c1ccc(-c2ccccc2-c2cccc(-c3nc(-c4cccc5c4oc4ccccc45)nc(-c4cccc5c4oc4ccccc45)n3)c2-c2ccccc2)cc1]

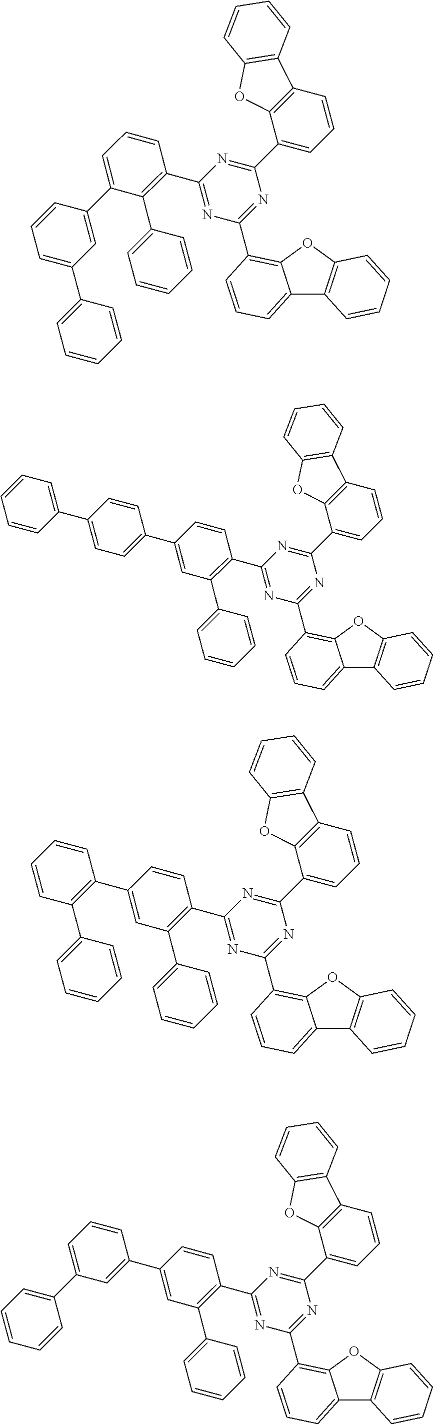 [SMILES: c1ccc(-c2cccc(-c3cccc(-c4nc(-c5cccc6c5oc5ccccc56)nc(-c5cccc6c5oc5ccccc56)n4)c3-c3ccccc3)c2)cc1 ; c1ccc(-c2ccc(-c3ccc(-c4nc(-c5cccc6c5oc5ccccc56)nc(-c5cccc6c5oc5ccccc56)n4)c(-c4ccccc4)c3)cc2)cc1 ; c1ccc(-c2ccccc2-c2ccc(-c3nc(-c4cccc5c4oc4ccccc45)nc(-c4cccc5c4oc4ccccc45)n3)c(-c3ccccc3)c2)cc1 ; c1ccc(-c2cccc(-c3ccc(-c4nc(-c5cccc6c5oc5ccccc56)nc(-c5cccc6c5oc5ccccc56)n4)c(-c4ccccc4)c3)c2)cc1]

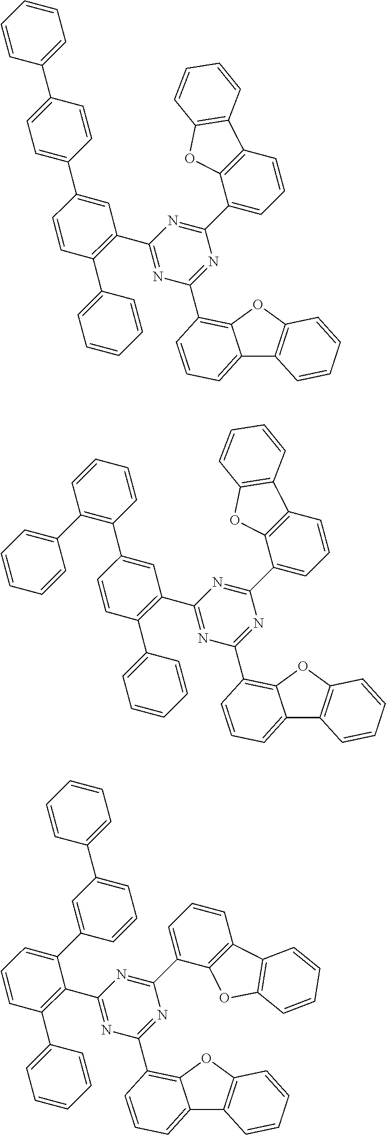 [SMILES: c1ccc(-c2ccc(-c3ccc(-c4ccccc4)c(-c4nc(-c5cccc6c5oc5ccccc56)nc(-c5cccc6c5oc5ccccc56)n4)c3)cc2)cc1 ; c1ccc(-c2ccccc2-c2ccc(-c3ccccc3)c(-c3nc(-c4cccc5c4oc4ccccc45)nc(-c4cccc5c4oc4ccccc45)n3)c2)cc1 ; c1ccc(-c2cccc(-c3cccc(-c4ccccc4)c3-c3nc(-c4cccc5c4oc4ccccc45)nc(-c4cccc5c4oc4ccccc45)n3)c2)cc1]

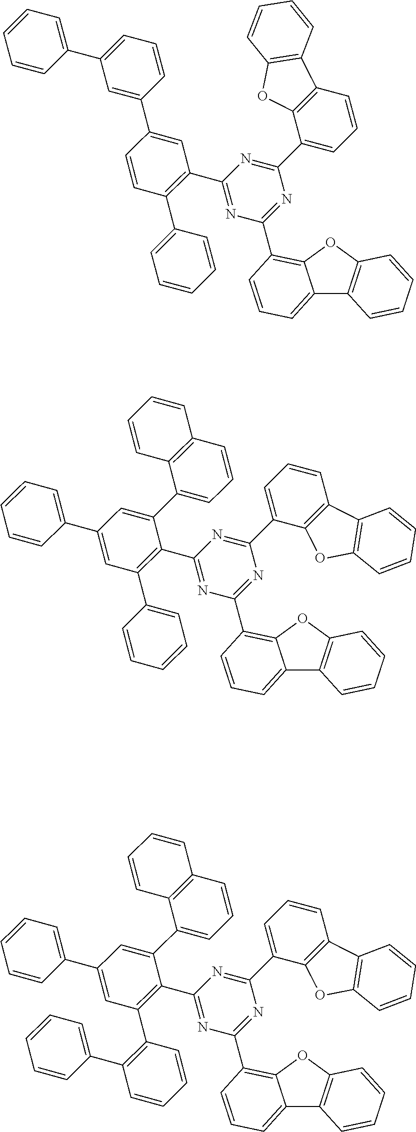 [SMILES: c1ccc(-c2cccc(-c3ccc(-c4ccccc4)c(-c4nc(-c5cccc6c5oc5ccccc56)nc(-c5cccc6c5oc5ccccc56)n4)c3)c2)cc1 ; c1ccc(-c2cc(-c3ccccc3)c(-c3nc(-c4cccc5c4oc4ccccc45)nc(-c4cccc5c4oc4ccccc45)n3)c(-c3cccc4ccccc34)c2)cc1 ; c1ccc(-c2cc(-c3ccccc3-c3ccccc3)c(-c3nc(-c4cccc5c4oc4ccccc45)nc(-c4cccc5c4oc4ccccc45)n3)c(-c3cccc4ccccc34)c2)cc1]

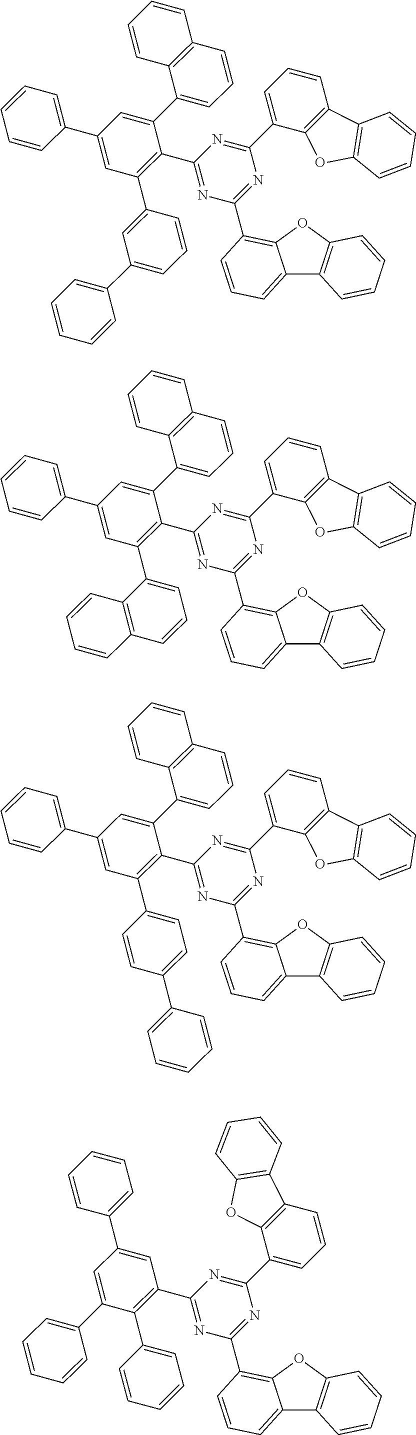 [SMILES: c1ccc(-c2cccc(-c3cc(-c4ccccc4)cc(-c4cccc5ccccc45)c3-c3nc(-c4cccc5c4oc4ccccc45)nc(-c4cccc5c4oc4ccccc45)n3)c2)cc1 ; c1ccc(-c2cc(-c3cccc4ccccc34)c(-c3nc(-c4cccc5c4oc4ccccc45)nc(-c4cccc5c4oc4ccccc45)n3)c(-c3cccc4ccccc34)c2)cc1 ; c1ccc(-c2ccc(-c3cc(-c4ccccc4)cc(-c4cccc5ccccc45)c3-c3nc(-c4cccc5c4oc4ccccc45)nc(-c4cccc5c4oc4ccccc45)n3)cc2)cc1 ; c1ccc(-c2cc(-c3ccccc3)c(-c3ccccc3)c(-c3nc(-c4cccc5c4oc4ccccc45)nc(-c4cccc5c4oc4ccccc45)n3)c2)cc1]

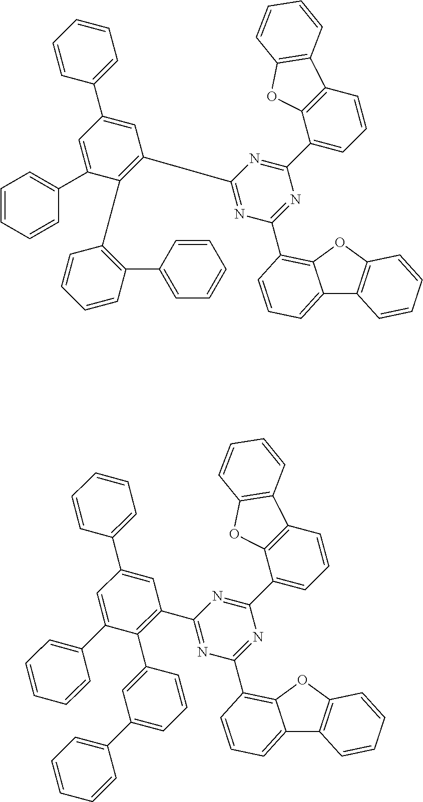 [SMILES: c1ccc(-c2cc(-c3ccccc3)c(-c3ccccc3-c3ccccc3)c(-c3nc(-c4cccc5c4oc4ccccc45)nc(-c4cccc5c4oc4ccccc45)n3)c2)cc1 ; c1ccc(-c2cccc(-c3c(-c4ccccc4)cc(-c4ccccc4)cc3-c3nc(-c4cccc5c4oc4ccccc45)nc(-c4cccc5c4oc4ccccc45)n3)c2)cc1]

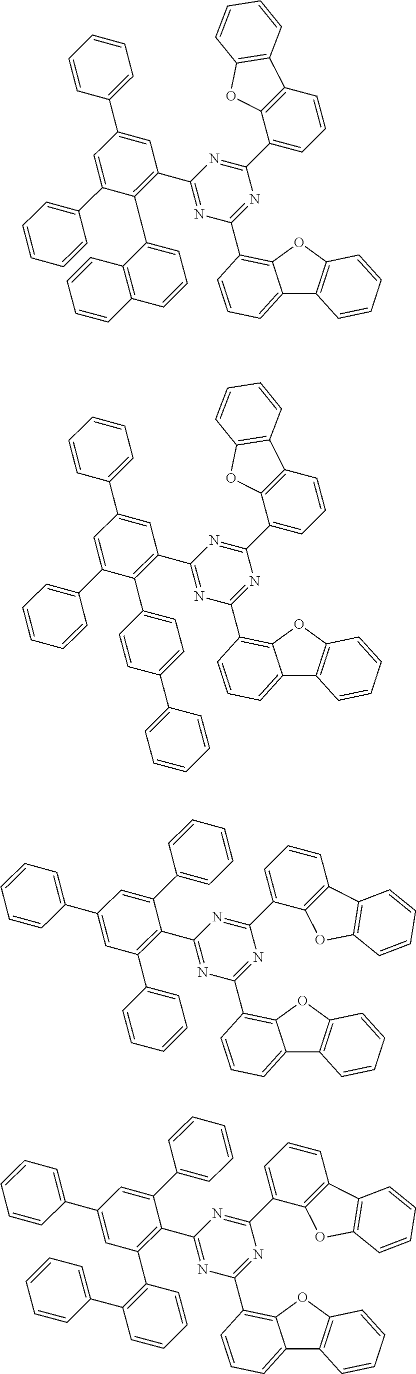 [SMILES: c1ccc(-c2cc(-c3ccccc3)c(-c3cccc4ccccc34)c(-c3nc(-c4cccc5c4oc4ccccc45)nc(-c4cccc5c4oc4ccccc45)n3)c2)cc1 ; c1ccc(-c2ccc(-c3c(-c4ccccc4)cc(-c4ccccc4)cc3-c3nc(-c4cccc5c4oc4ccccc45)nc(-c4cccc5c4oc4ccccc45)n3)cc2)cc1 ; c1ccc(-c2cc(-c3ccccc3)c(-c3nc(-c4cccc5c4oc4ccccc45)nc(-c4cccc5c4oc4ccccc45)n3)c(-c3ccccc3)c2)cc1 ; c1ccc(-c2cc(-c3ccccc3)c(-c3nc(-c4cccc5c4oc4ccccc45)nc(-c4cccc5c4oc4ccccc45)n3)c(-c3ccccc3-c3ccccc3)c2)cc1]

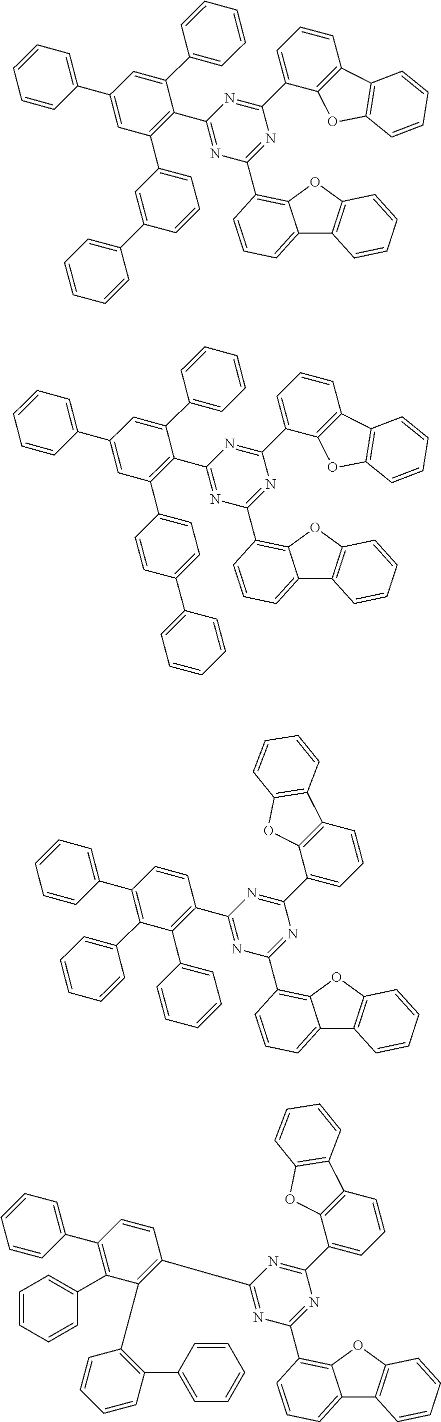 [SMILES: c1ccc(-c2cccc(-c3cc(-c4ccccc4)cc(-c4ccccc4)c3-c3nc(-c4cccc5c4oc4ccccc45)nc(-c4cccc5c4oc4ccccc45)n3)c2)cc1 ; c1ccc(-c2ccc(-c3cc(-c4ccccc4)cc(-c4ccccc4)c3-c3nc(-c4cccc5c4oc4ccccc45)nc(-c4cccc5c4oc4ccccc45)n3)cc2)cc1 ; c1ccc(-c2ccc(-c3nc(-c4cccc5c4oc4ccccc45)nc(-c4cccc5c4oc4ccccc45)n3)c(-c3ccccc3)c2-c2ccccc2)cc1 ; c1ccc(-c2ccccc2-c2c(-c3nc(-c4cccc5c4oc4ccccc45)nc(-c4cccc5c4oc4ccccc45)n3)ccc(-c3ccccc3)c2-c2ccccc2)cc1]

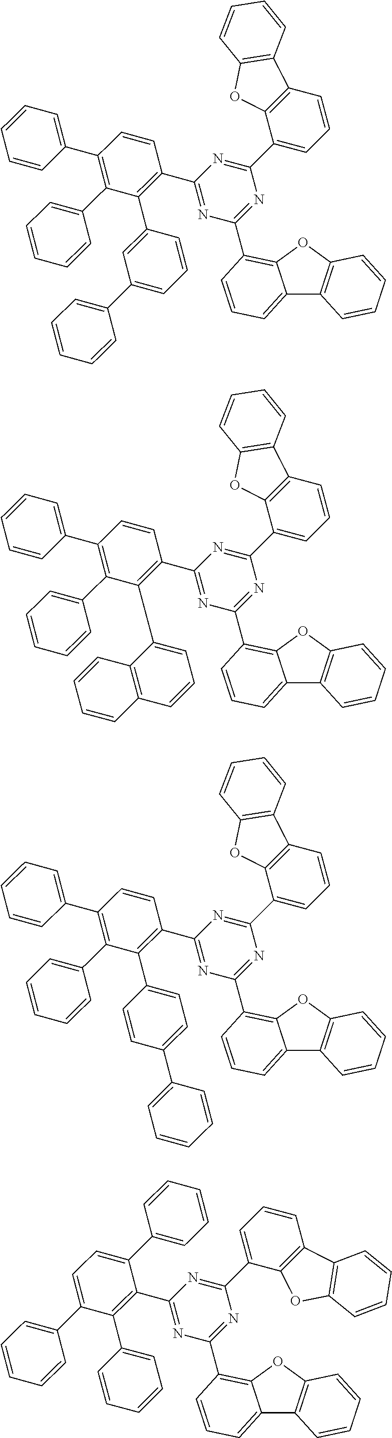

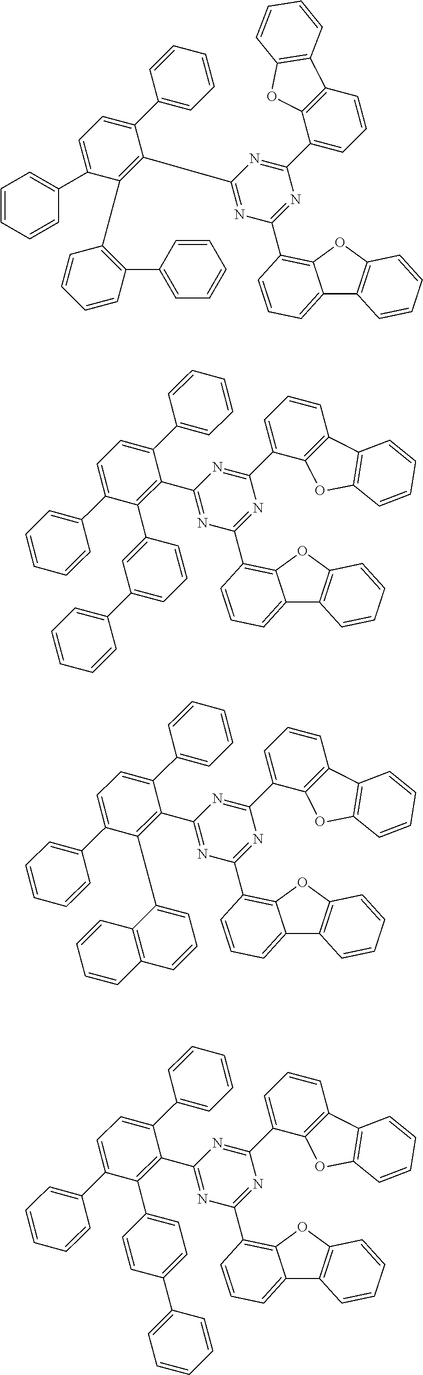 [SMILES: c1ccc(-c2ccccc2-c2c(-c3ccccc3)ccc(-c3ccccc3)c2-c2nc(-c3cccc4c3oc3ccccc34)nc(-c3cccc4c3oc3ccccc34)n2)cc1 ; c1ccc(-c2cccc(-c3c(-c4ccccc4)ccc(-c4ccccc4)c3-c3nc(-c4cccc5c4oc4ccccc45)nc(-c4cccc5c4oc4ccccc45)n3)c2)cc1 ; c1ccc(-c2ccc(-c3ccccc3)c(-c3cccc4ccccc34)c2-c2nc(-c3cccc4c3oc3ccccc34)nc(-c3cccc4c3oc3ccccc34)n2)cc1 ; c1ccc(-c2ccc(-c3c(-c4ccccc4)ccc(-c4ccccc4)c3-c3nc(-c4cccc5c4oc4ccccc45)nc(-c4cccc5c4oc4ccccc45)n3)cc2)cc1]

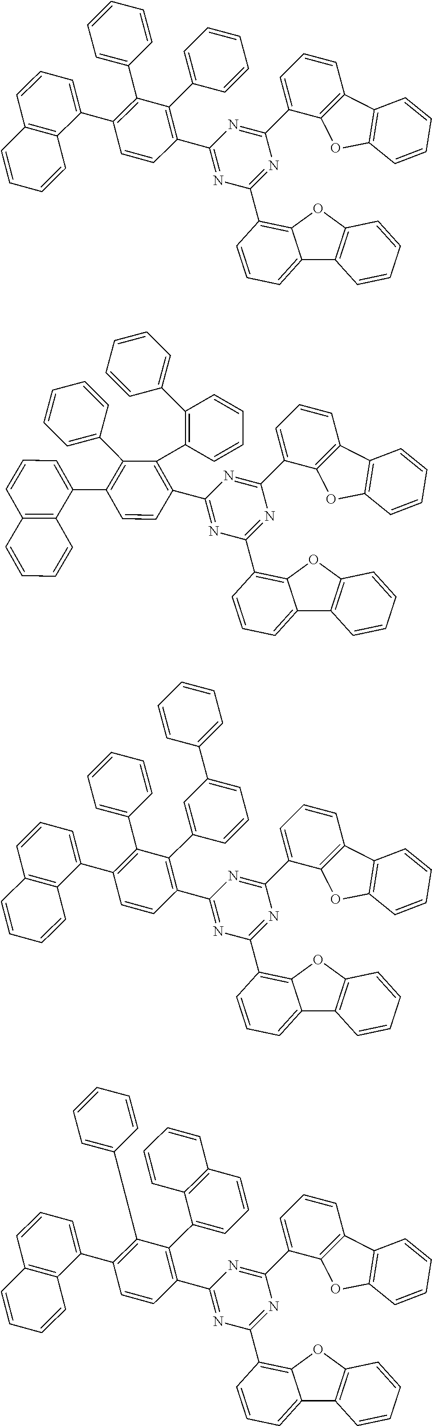 [SMILES: c1ccc(-c2c(-c3nc(-c4cccc5c4oc4ccccc45)nc(-c4cccc5c4oc4ccccc45)n3)ccc(-c3cccc4ccccc34)c2-c2ccccc2)cc1 ; c1ccc(-c2ccccc2-c2c(-c3nc(-c4cccc5c4oc4ccccc45)nc(-c4cccc5c4oc4ccccc45)n3)ccc(-c3cccc4ccccc34)c2-c2ccccc2)cc1 ; c1ccc(-c2cccc(-c3c(-c4nc(-c5cccc6c5oc5ccccc56)nc(-c5cccc6c5oc5ccccc56)n4)ccc(-c4cccc5ccccc45)c3-c3ccccc3)c2)cc1 ; c1ccc(-c2c(-c3cccc4ccccc34)ccc(-c3nc(-c4cccc5c4oc4ccccc45)nc(-c4cccc5c4oc4ccccc45)n3)c2-c2cccc3ccccc23)cc1]

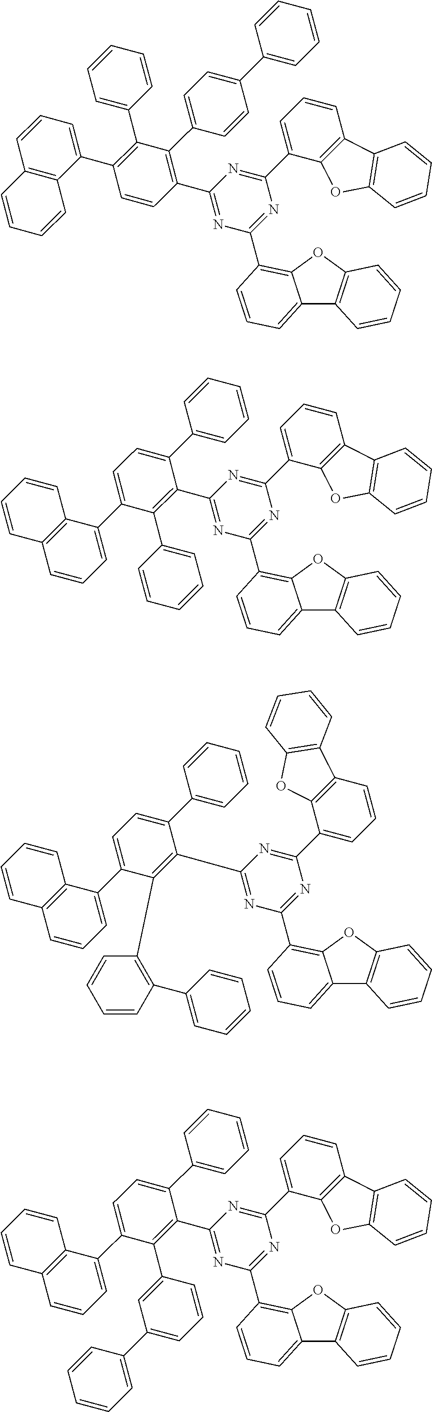 [SMILES: c1ccc(-c2ccc(-c3c(-c4nc(-c5cccc6c5oc5ccccc56)nc(-c5cccc6c5oc5ccccc56)n4)ccc(-c4cccc5ccccc45)c3-c3ccccc3)cc2)cc1 ; c1ccc(-c2ccc(-c3cccc4ccccc34)c(-c3ccccc3)c2-c2nc(-c3cccc4c3oc3ccccc34)nc(-c3cccc4c3oc3ccccc34)n2)cc1 ; c1ccc(-c2ccccc2-c2c(-c3cccc4ccccc34)ccc(-c3ccccc3)c2-c2nc(-c3cccc4c3oc3ccccc34)nc(-c3cccc4c3oc3ccccc34)n2)cc1 ; c1ccc(-c2cccc(-c3c(-c4cccc5ccccc45)ccc(-c4ccccc4)c3-c3nc(-c4cccc5c4oc4ccccc45)nc(-c4cccc5c4oc4ccccc45)n3)c2)cc1]

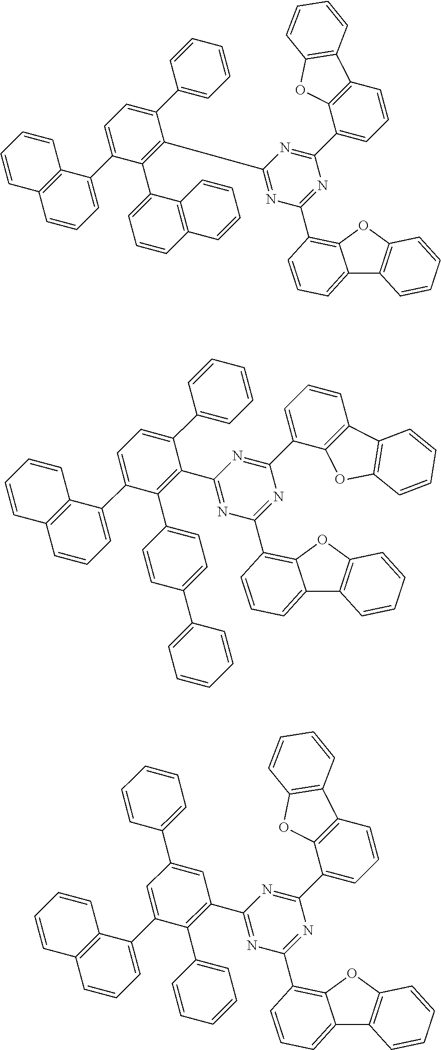 [SMILES: c1ccc(-c2ccc(-c3cccc4ccccc34)c(-c3cccc4ccccc34)c2-c2nc(-c3cccc4c3oc3ccccc34)nc(-c3cccc4c3oc3ccccc34)n2)cc1 ; c1ccc(-c2ccc(-c3c(-c4cccc5ccccc45)ccc(-c4ccccc4)c3-c3nc(-c4cccc5c4oc4ccccc45)nc(-c4cccc5c4oc4ccccc45)n3)cc2)cc1 ; c1ccc(-c2cc(-c3nc(-c4cccc5c4oc4ccccc45)nc(-c4cccc5c4oc4ccccc45)n3)c(-c3ccccc3)c(-c3cccc4ccccc34)c2)cc1]

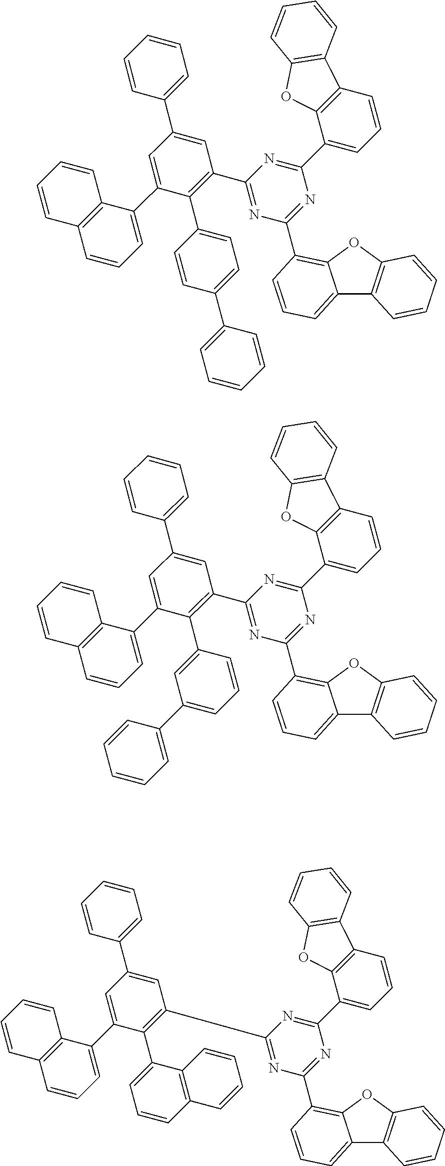 [SMILES: c1ccc(-c2ccc(-c3c(-c4nc(-c5cccc6c5oc5ccccc56)nc(-c5cccc6c5oc5ccccc56)n4)cc(-c4ccccc4)cc3-c3cccc4ccccc34)cc2)cc1 ; c1ccc(-c2cccc(-c3c(-c4nc(-c5cccc6c5oc5ccccc56)nc(-c5cccc6c5oc5ccccc56)n4)cc(-c4ccccc4)cc3-c3cccc4ccccc34)c2)cc1 ; c1ccc(-c2cc(-c3nc(-c4cccc5c4oc4ccccc45)nc(-c4cccc5c4oc4ccccc45)n3)c(-c3cccc4ccccc34)c(-c3cccc4ccccc34)c2)cc1]

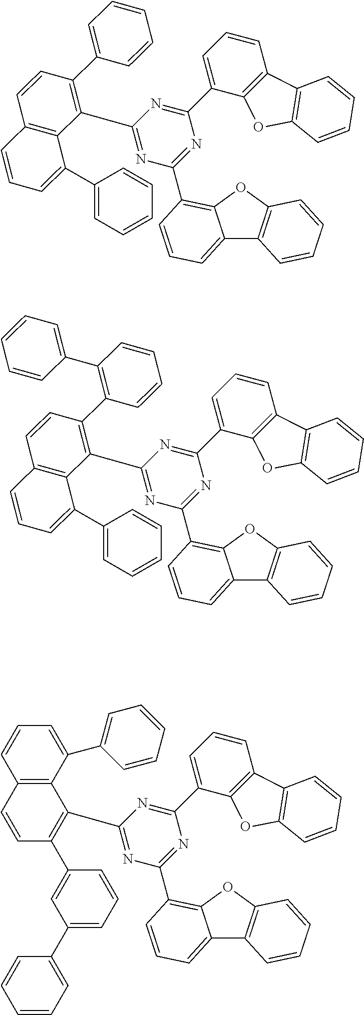 [SMILES: c1ccc(-c2ccc3cccc(-c4ccccc4)c3c2-c2nc(-c3cccc4c3oc3ccccc34)nc(-c3cccc4c3oc3ccccc34)n2)cc1 ; c1ccc(-c2ccccc2-c2ccc3cccc(-c4ccccc4)c3c2-c2nc(-c3cccc4c3oc3ccccc34)nc(-c3cccc4c3oc3ccccc34)n2)cc1 ; c1ccc(-c2cccc(-c3ccc4cccc(-c5ccccc5)c4c3-c3nc(-c4cccc5c4oc4ccccc45)nc(-c4cccc5c4oc4ccccc45)n3)c2)cc1]

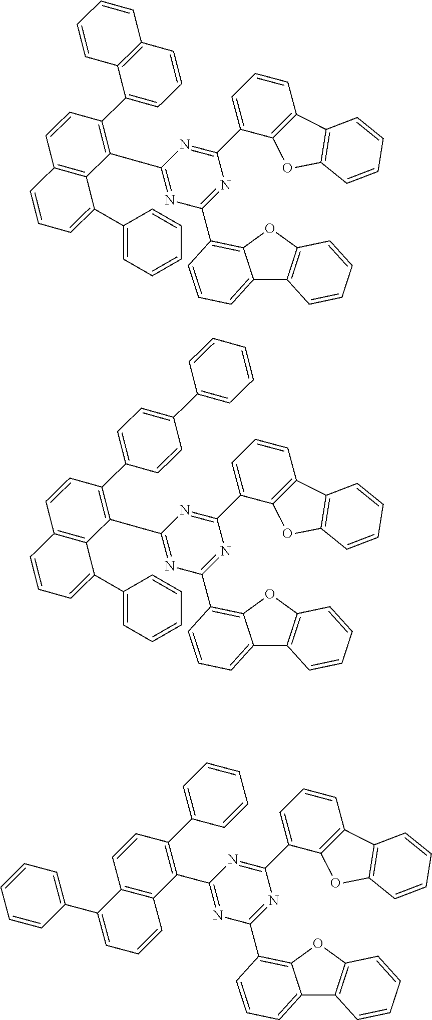

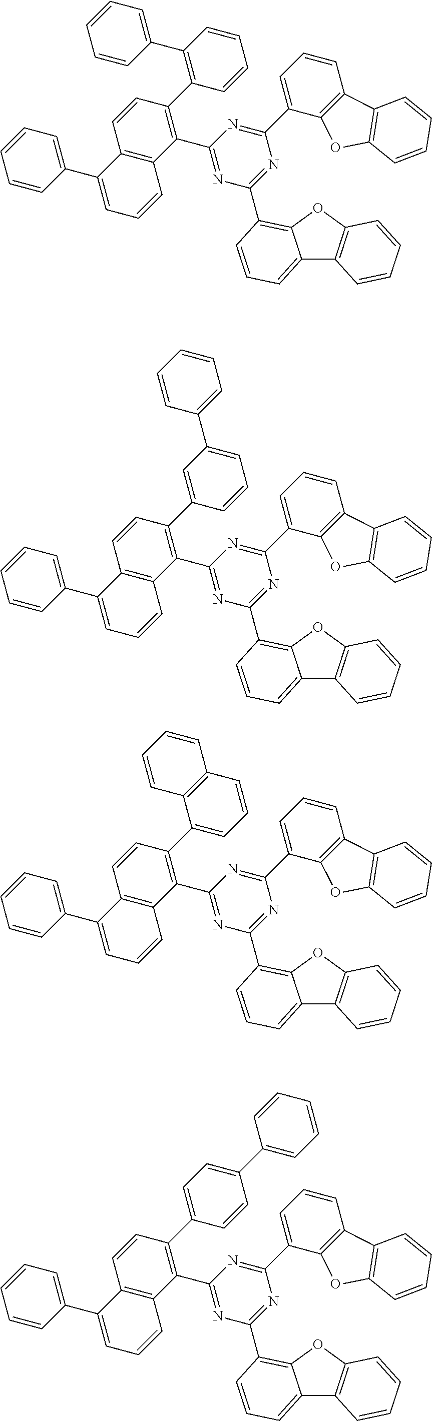 [SMILES: c1ccc(-c2ccccc2-c2ccc3c(-c4ccccc4)cccc3c2-c2nc(-c3cccc4c3oc3ccccc34)nc(-c3cccc4c3oc3ccccc34)n2)cc1 ; c1ccc(-c2cccc(-c3ccc4c(-c5ccccc5)cccc4c3-c3nc(-c4cccc5c4oc4ccccc45)nc(-c4cccc5c4oc4ccccc45)n3)c2)cc1 ; c1ccc(-c2cccc3c(-c4nc(-c5cccc6c5oc5ccccc56)nc(-c5cccc6c5oc5ccccc56)n4)c(-c4cccc5ccccc45)ccc23)cc1 ; c1ccc(-c2ccc(-c3ccc4c(-c5ccccc5)cccc4c3-c3nc(-c4cccc5c4oc4ccccc45)nc(-c4cccc5c4oc4ccccc45)n3)cc2)cc1]

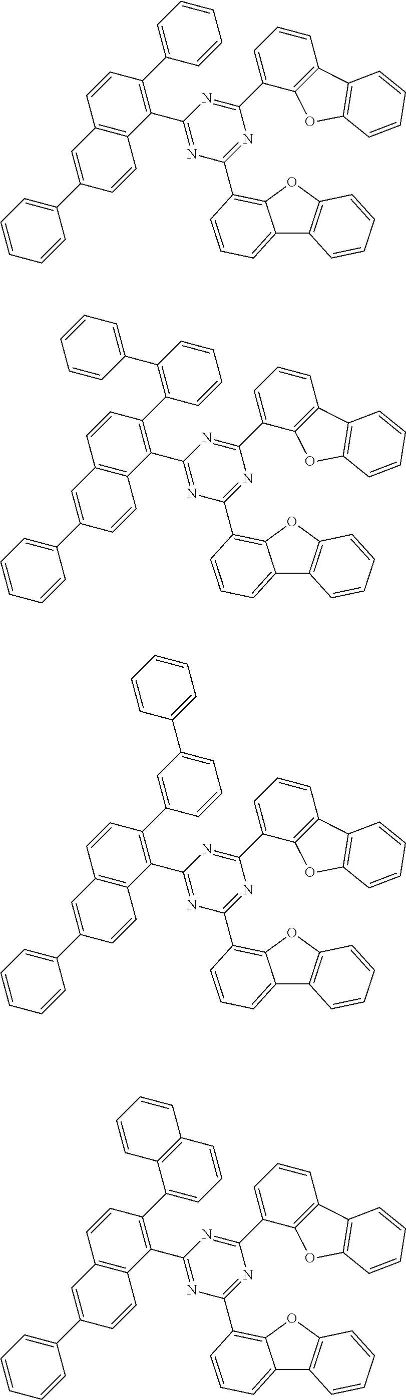 [SMILES: c1ccc(-c2ccc3c(-c4nc(-c5cccc6c5oc5ccccc56)nc(-c5cccc6c5oc5ccccc56)n4)c(-c4ccccc4)ccc3c2)cc1 ; c1ccc(-c2ccc3c(-c4nc(-c5cccc6c5oc5ccccc56)nc(-c5cccc6c5oc5ccccc56)n4)c(-c4ccccc4-c4ccccc4)ccc3c2)cc1 ; c1ccc(-c2cccc(-c3ccc4cc(-c5ccccc5)ccc4c3-c3nc(-c4cccc5c4oc4ccccc45)nc(-c4cccc5c4oc4ccccc45)n3)c2)cc1 ; c1ccc(-c2ccc3c(-c4nc(-c5cccc6c5oc5ccccc56)nc(-c5cccc6c5oc5ccccc56)n4)c(-c4cccc5ccccc45)ccc3c2)cc1]

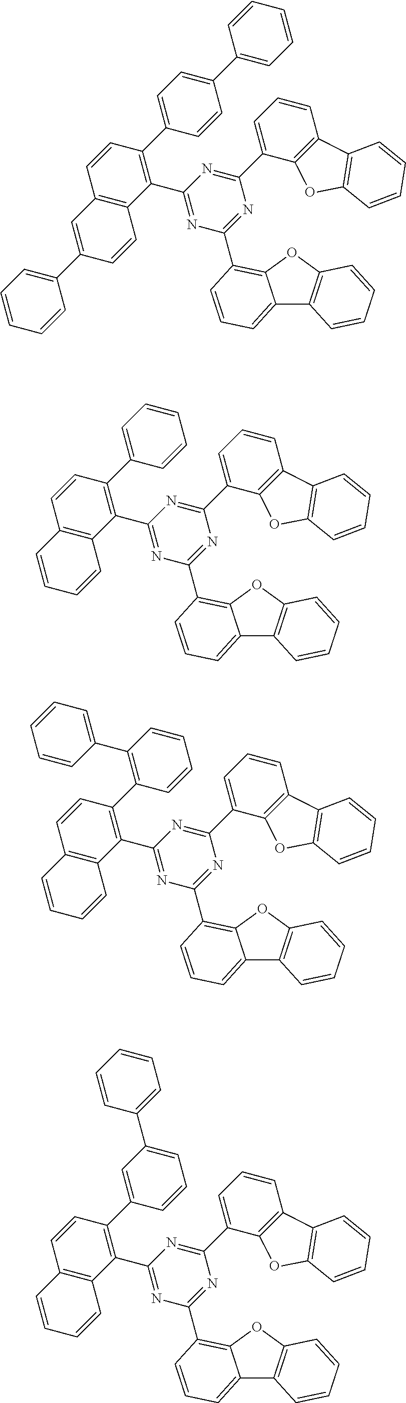 [SMILES: c1ccc(-c2ccc(-c3ccc4cc(-c5ccccc5)ccc4c3-c3nc(-c4cccc5c4oc4ccccc45)nc(-c4cccc5c4oc4ccccc45)n3)cc2)cc1 ; c1ccc(-c2ccc3ccccc3c2-c2nc(-c3cccc4c3oc3ccccc34)nc(-c3cccc4c3oc3ccccc34)n2)cc1 ; c1ccc(-c2ccccc2-c2ccc3ccccc3c2-c2nc(-c3cccc4c3oc3ccccc34)nc(-c3cccc4c3oc3ccccc34)n2)cc1 ; c1ccc(-c2cccc(-c3ccc4ccccc4c3-c3nc(-c4cccc5c4oc4ccccc45)nc(-c4cccc5c4oc4ccccc45)n3)c2)cc1]

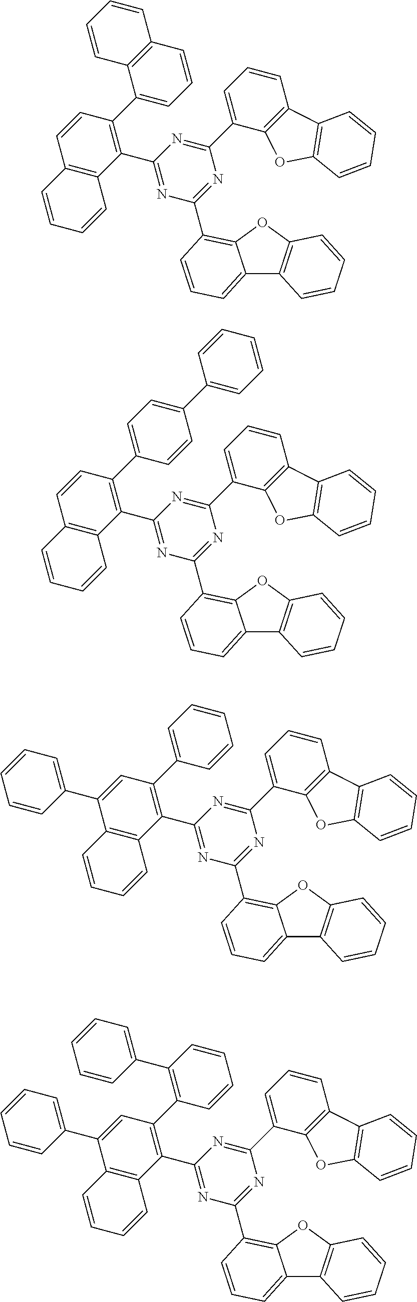

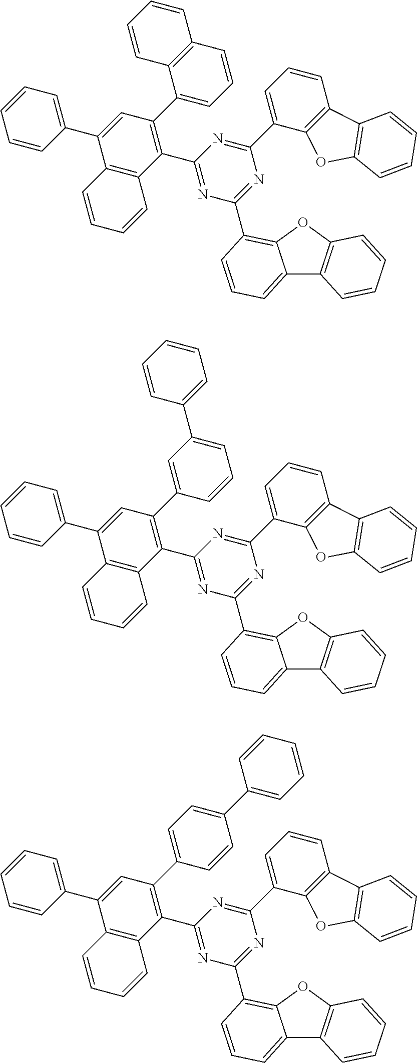

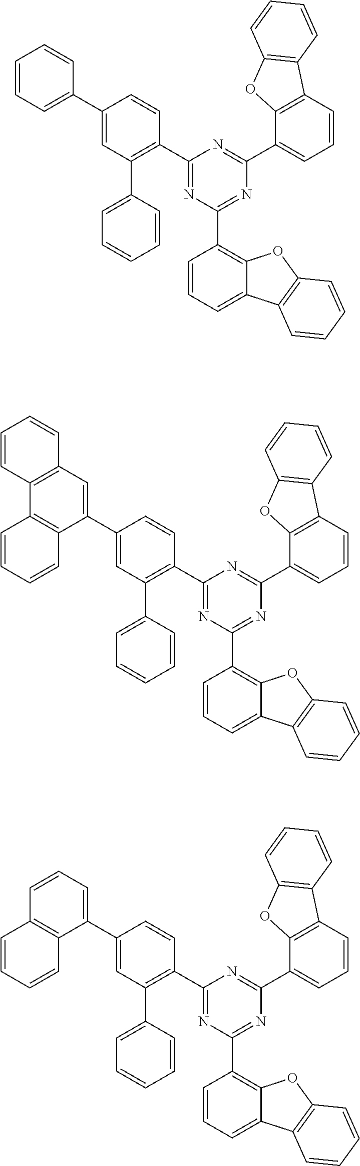 [SMILES: c1ccc(-c2ccc(-c3nc(-c4cccc5c4oc4ccccc45)nc(-c4cccc5c4oc4ccccc45)n3)c(-c3ccccc3)c2)cc1 ; c1ccc(-c2cc(-c3cc4ccccc4c4ccccc34)ccc2-c2nc(-c3cccc4c3oc3ccccc34)nc(-c3cccc4c3oc3ccccc34)n2)cc1 ; c1ccc(-c2cc(-c3cccc4ccccc34)ccc2-c2nc(-c3cccc4c3oc3ccccc34)nc(-c3cccc4c3oc3ccccc34)n2)cc1]

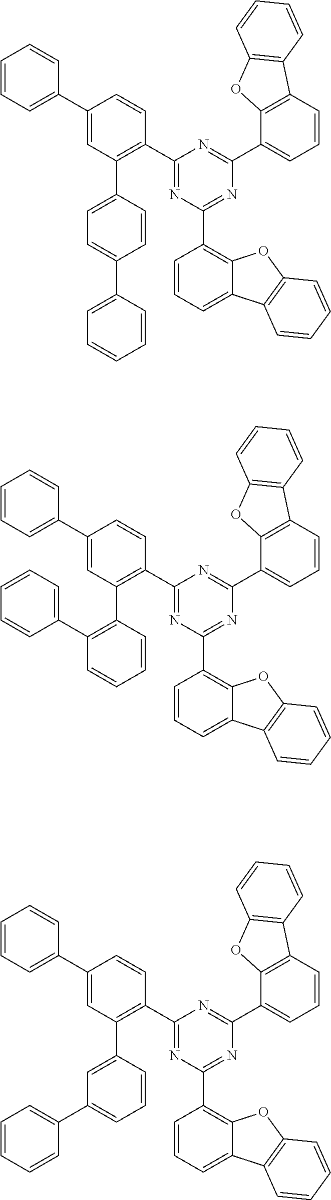 [SMILES: c1ccc(-c2ccc(-c3cc(-c4ccccc4)ccc3-c3nc(-c4cccc5c4oc4ccccc45)nc(-c4cccc5c4oc4ccccc45)n3)cc2)cc1 ; c1ccc(-c2ccc(-c3nc(-c4cccc5c4oc4ccccc45)nc(-c4cccc5c4oc4ccccc45)n3)c(-c3ccccc3-c3ccccc3)c2)cc1 ; c1ccc(-c2cccc(-c3cc(-c4ccccc4)ccc3-c3nc(-c4cccc5c4oc4ccccc45)nc(-c4cccc5c4oc4ccccc45)n3)c2)cc1]

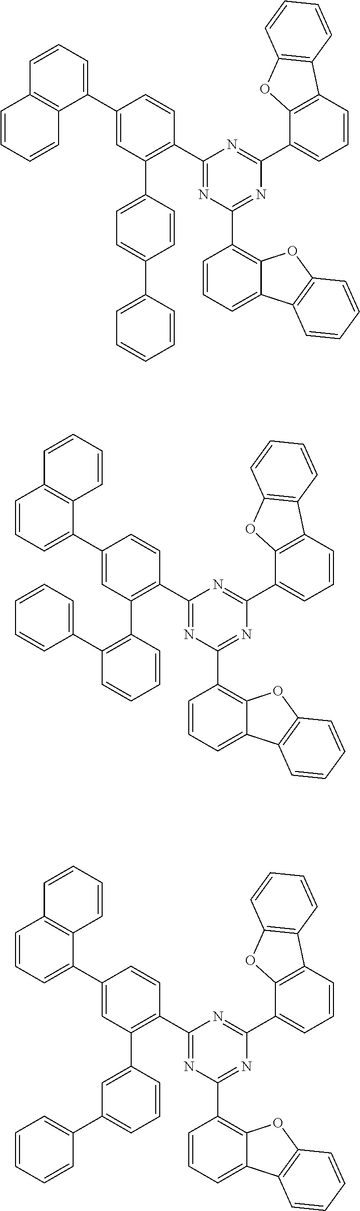 [SMILES: c1ccc(-c2ccc(-c3cc(-c4cccc5ccccc45)ccc3-c3nc(-c4cccc5c4oc4ccccc45)nc(-c4cccc5c4oc4ccccc45)n3)cc2)cc1 ; c1ccc(-c2ccccc2-c2cc(-c3cccc4ccccc34)ccc2-c2nc(-c3cccc4c3oc3ccccc34)nc(-c3cccc4c3oc3ccccc34)n2)cc1 ; c1ccc(-c2cccc(-c3cc(-c4cccc5ccccc45)ccc3-c3nc(-c4cccc5c4oc4ccccc45)nc(-c4cccc5c4oc4ccccc45)n3)c2)cc1]

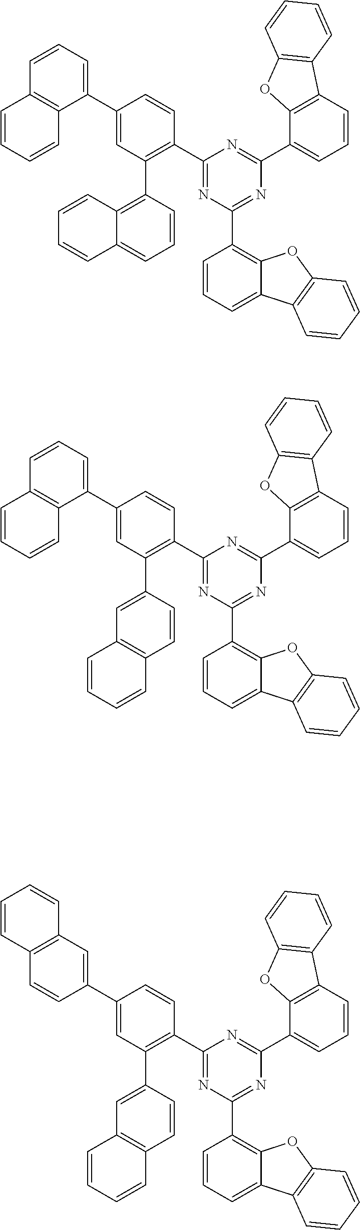 [SMILES: c1ccc2c(-c3ccc(-c4nc(-c5cccc6c5oc5ccccc56)nc(-c5cccc6c5oc5ccccc56)n4)c(-c4cccc5ccccc45)c3)cccc2c1 ; c1ccc2cc(-c3cc(-c4cccc5ccccc45)ccc3-c3nc(-c4cccc5c4oc4ccccc45)nc(-c4cccc5c4oc4ccccc45)n3)ccc2c1 ; c1ccc2cc(-c3ccc(-c4nc(-c5cccc6c5oc5ccccc56)nc(-c5cccc6c5oc5ccccc56)n4)c(-c4ccc5ccccc5c4)c3)ccc2c1]

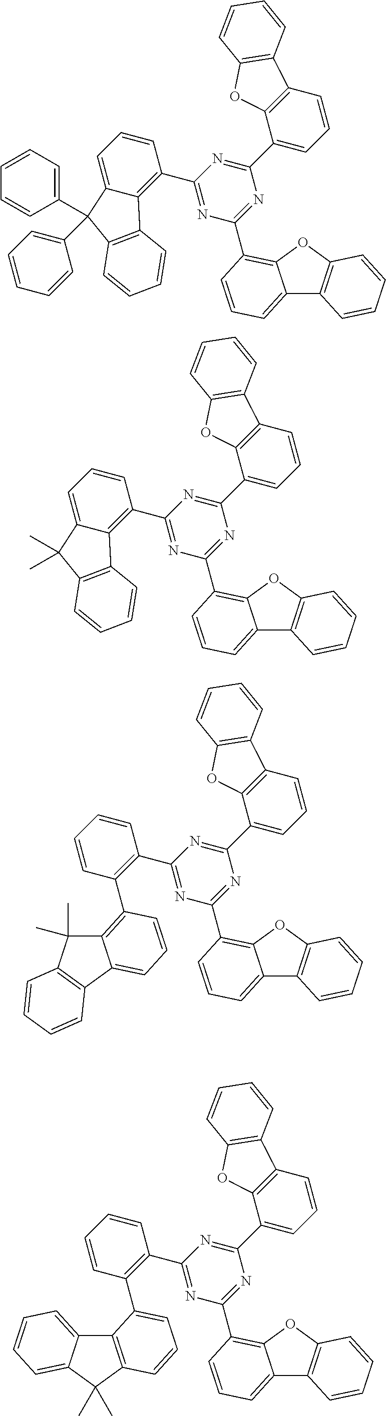

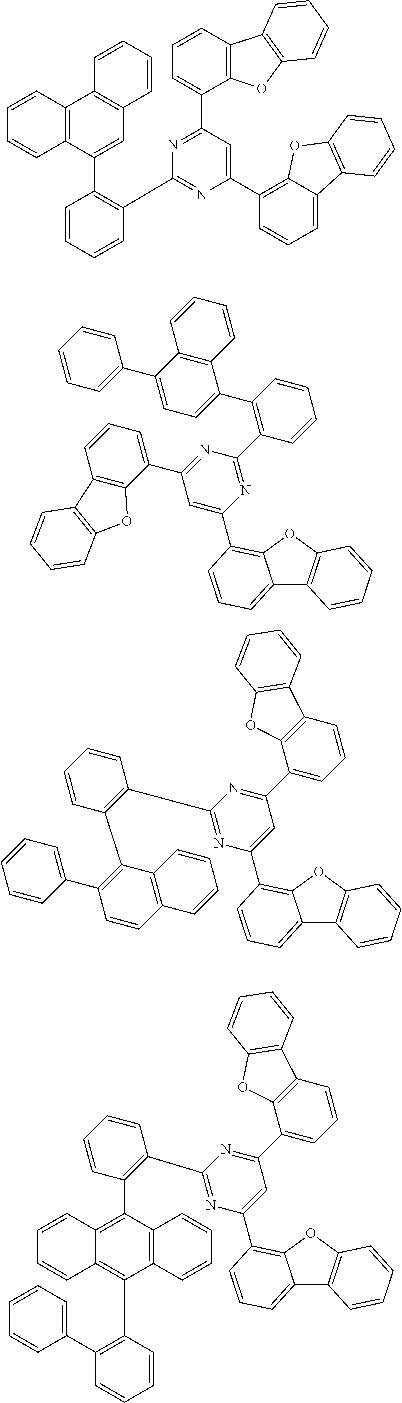

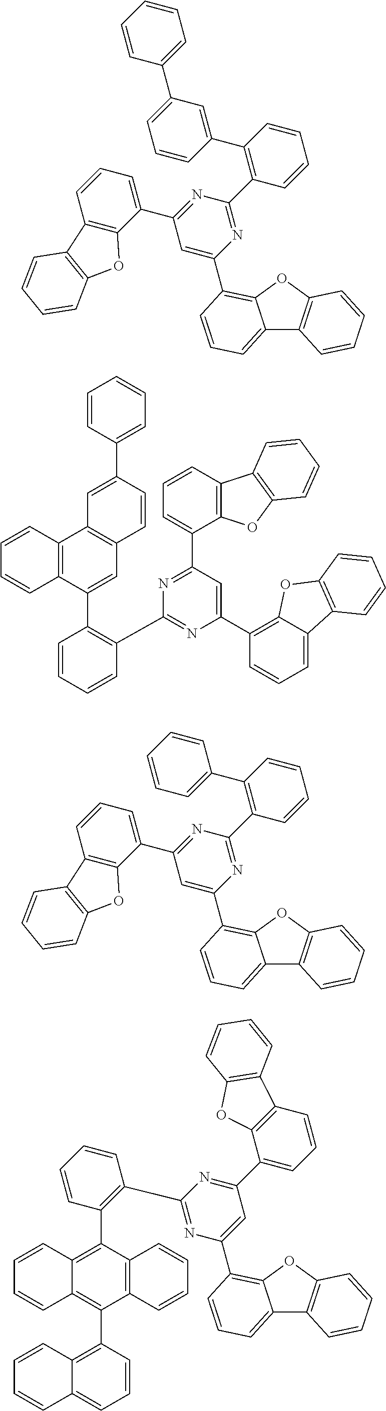

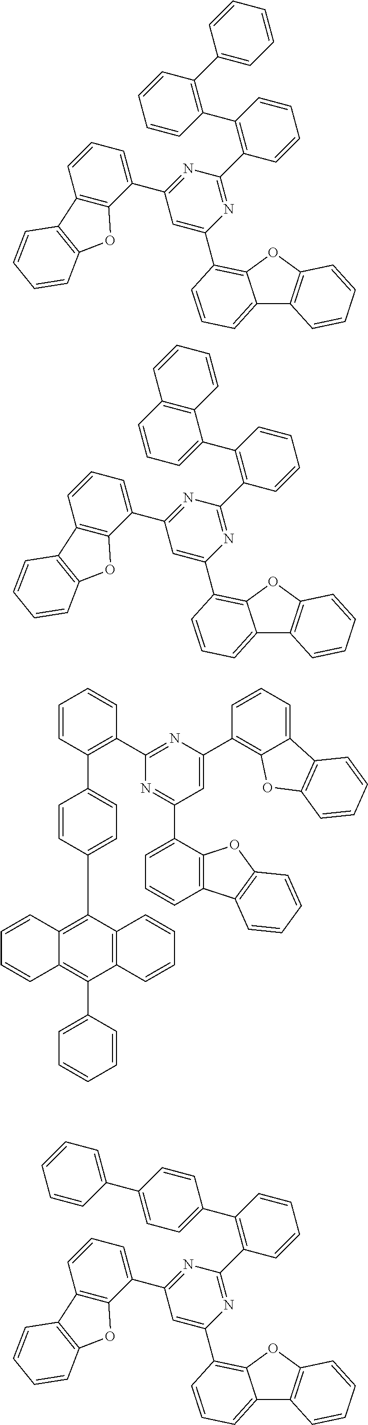

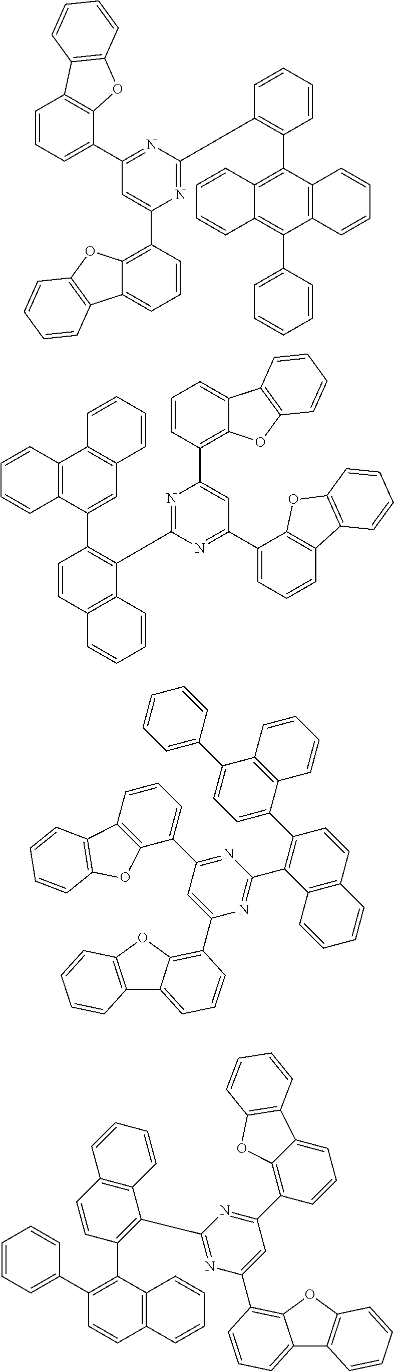

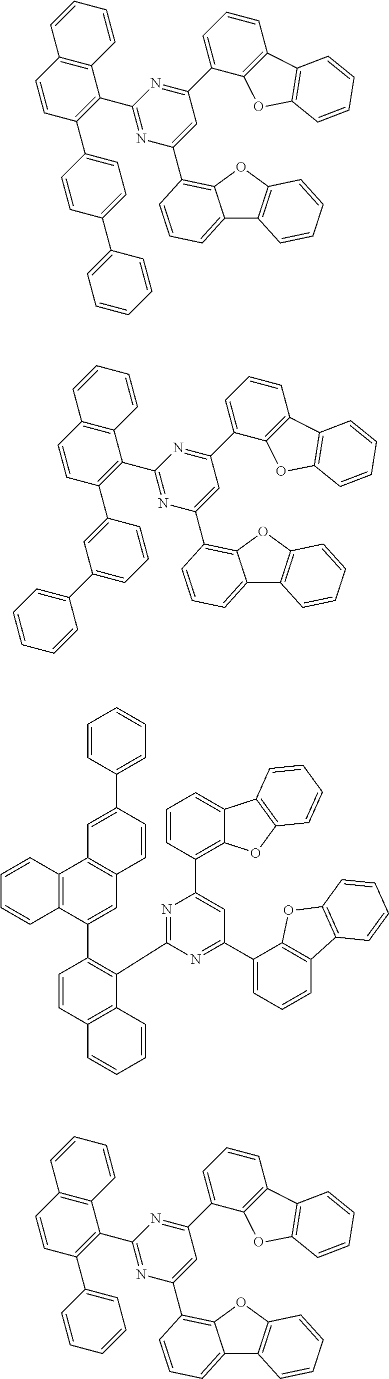 [SMILES: c1ccc(-c2ccc(-c3ccc4ccccc4c3-c3nc(-c4cccc5c4oc4ccccc45)cc(-c4cccc5c4oc4ccccc45)n3)cc2)cc1 ; c1ccc(-c2cccc(-c3ccc4ccccc4c3-c3nc(-c4cccc5c4oc4ccccc45)cc(-c4cccc5c4oc4ccccc45)n3)c2)cc1 ; c1ccc(-c2ccc3cc(-c4ccc5ccccc5c4-c4nc(-c5cccc6c5oc5ccccc56)cc(-c5cccc6c5oc5ccccc56)n4)c4ccccc4c3c2)cc1 ; c1ccc(-c2ccc3ccccc3c2-c2nc(-c3cccc4c3oc3ccccc34)cc(-c3cccc4c3oc3ccccc34)n2)cc1]

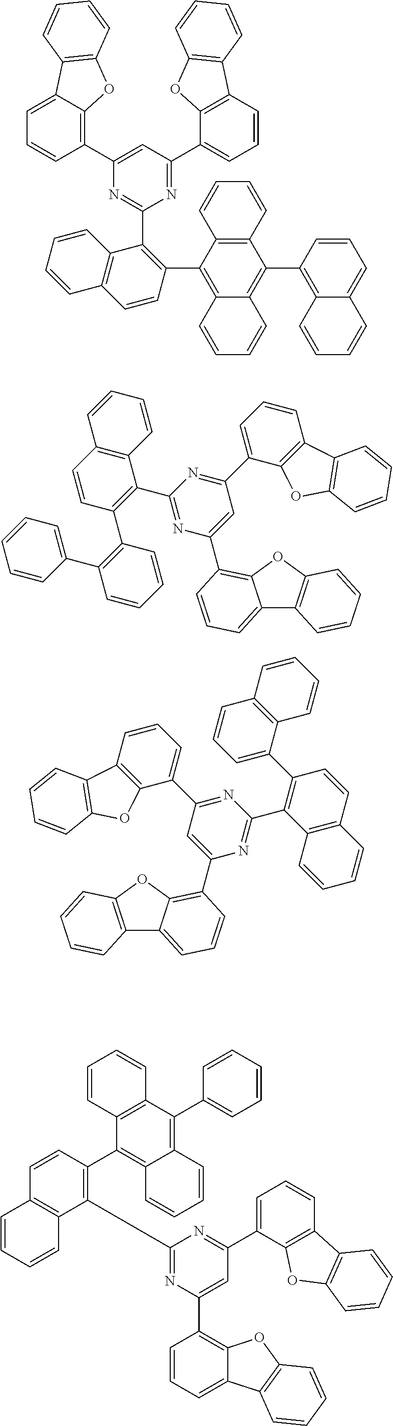

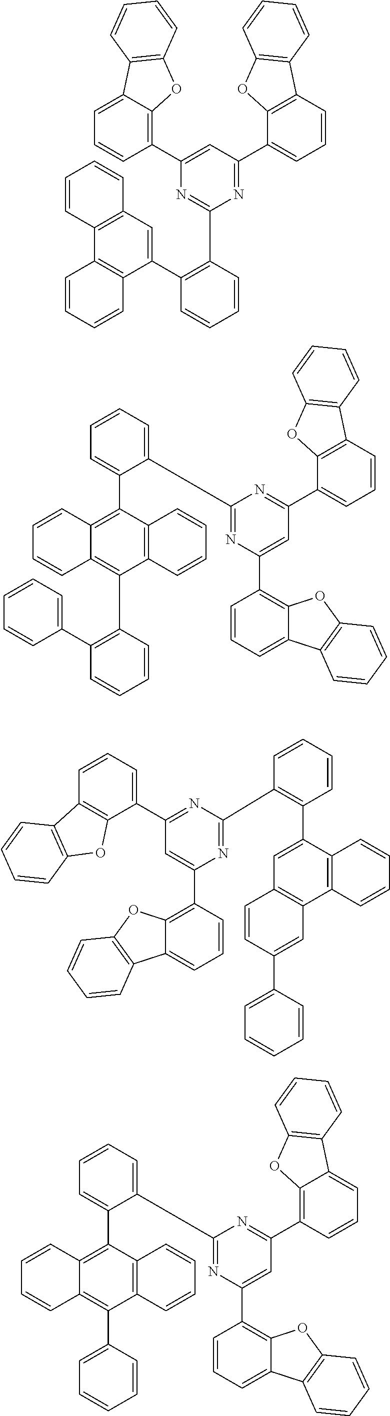

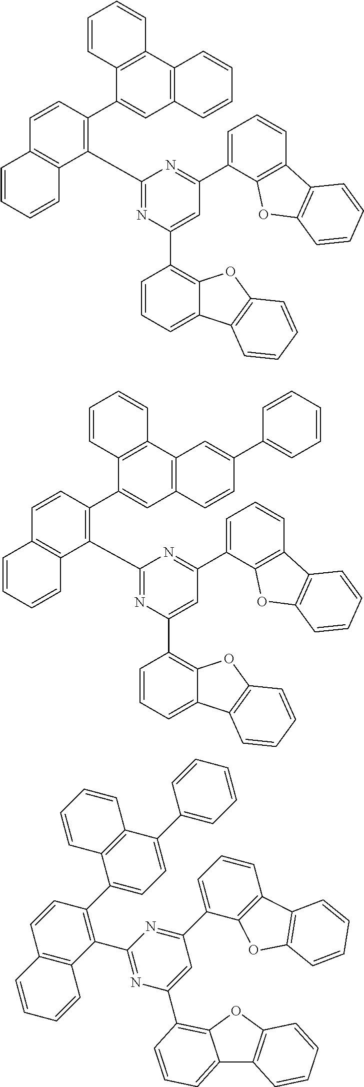

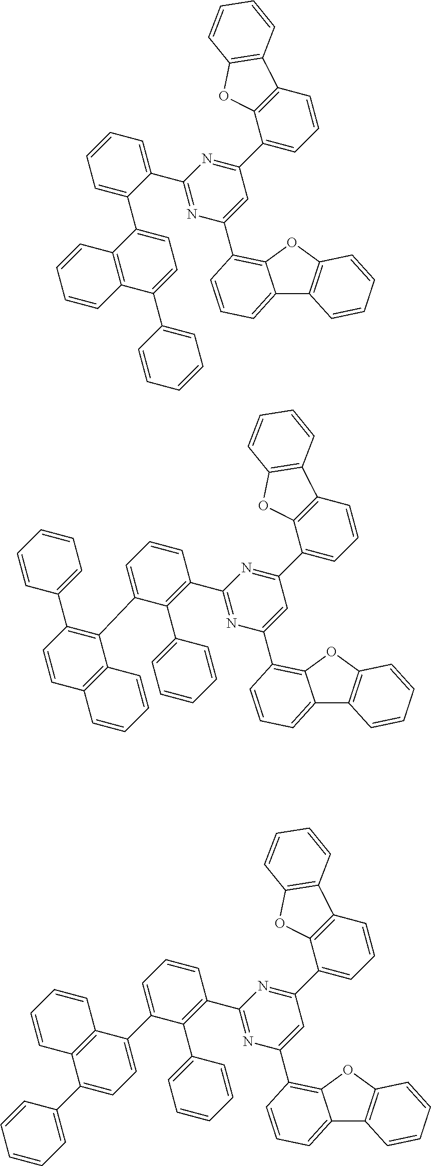 [SMILES: c1ccc(-c2ccc(-c3ccccc3-c3nc(-c4cccc5c4oc4ccccc45)cc(-c4cccc5c4oc4ccccc45)n3)c3ccccc23)cc1 ; c1ccc(-c2ccc3ccccc3c2-c2cccc(-c3nc(-c4cccc5c4oc4ccccc45)cc(-c4cccc5c4oc4ccccc45)n3)c2-c2ccccc2)cc1 ; c1ccc(-c2c(-c3nc(-c4cccc5c4oc4ccccc45)cc(-c4cccc5c4oc4ccccc45)n3)cccc2-c2ccc(-c3ccccc3)c3ccccc23)cc1]

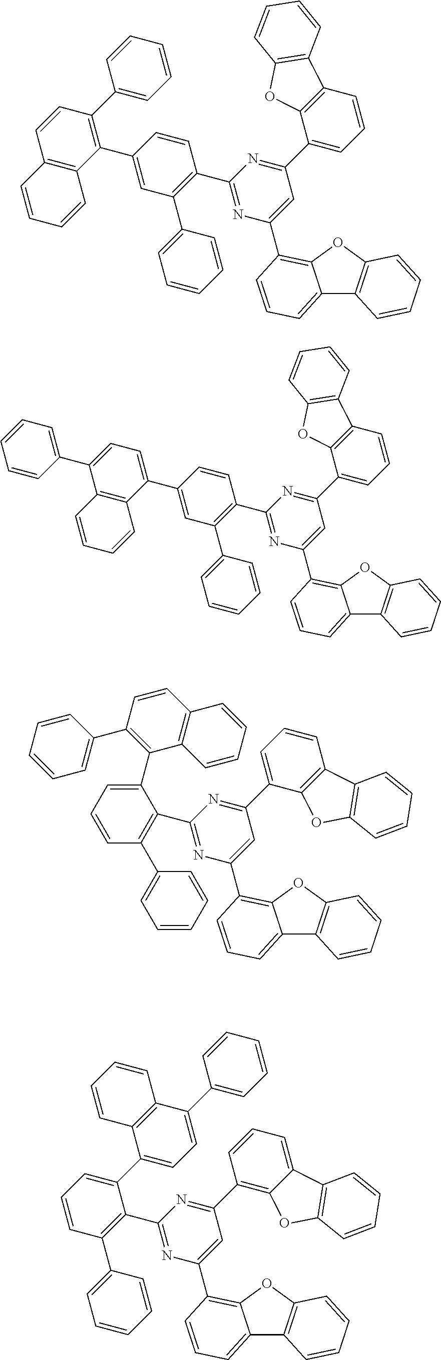

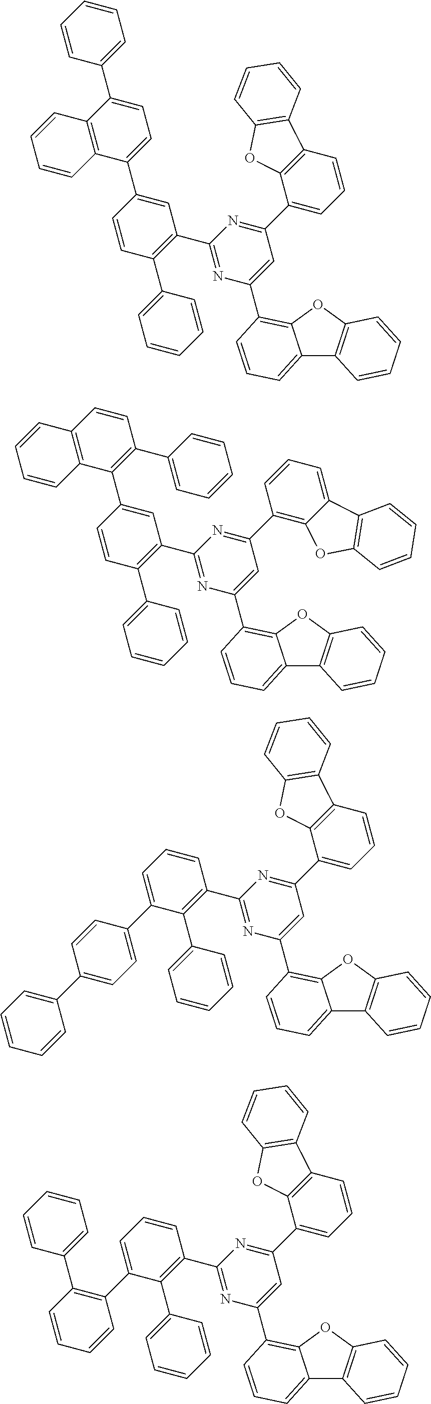

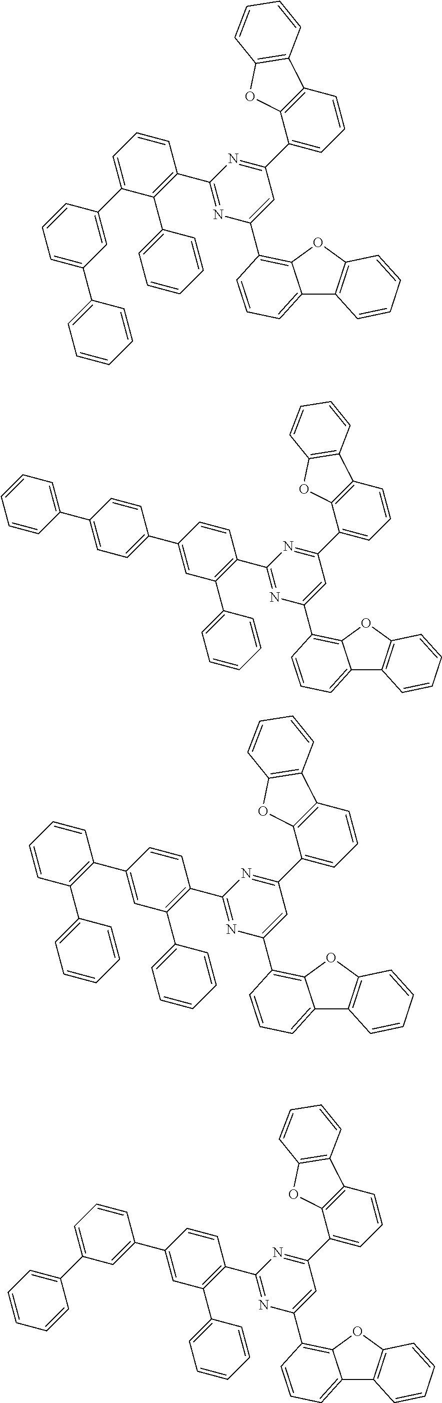

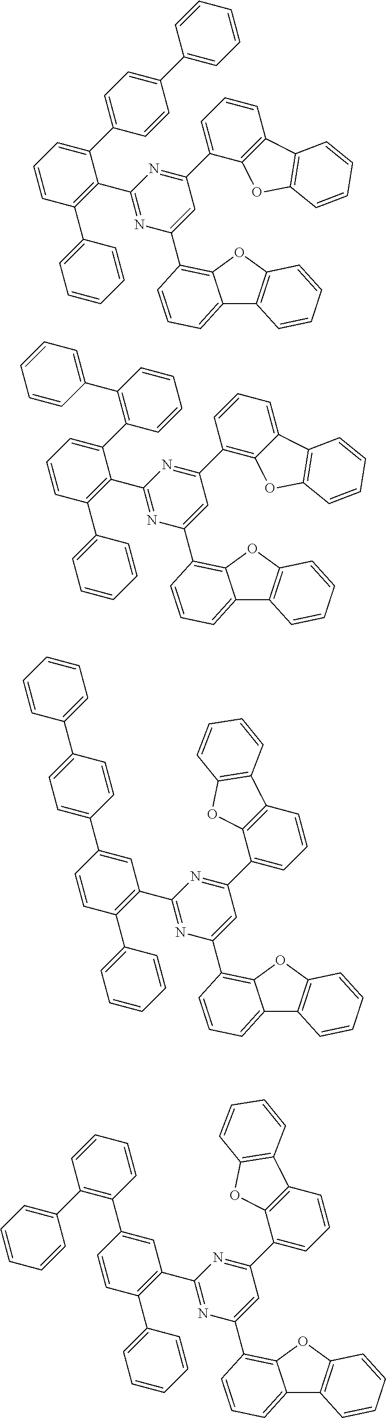 [SMILES: c1ccc(-c2ccc(-c3cccc(-c4ccccc4)c3-c3nc(-c4cccc5c4oc4ccccc45)cc(-c4cccc5c4oc4ccccc45)n3)cc2)cc1 ; c1ccc(-c2ccccc2-c2cccc(-c3ccccc3)c2-c2nc(-c3cccc4c3oc3ccccc34)cc(-c3cccc4c3oc3ccccc34)n2)cc1 ; c1ccc(-c2ccc(-c3ccc(-c4ccccc4)c(-c4nc(-c5cccc6c5oc5ccccc56)cc(-c5cccc6c5oc5ccccc56)n4)c3)cc2)cc1 ; c1ccc(-c2ccccc2-c2ccc(-c3ccccc3)c(-c3nc(-c4cccc5c4oc4ccccc45)cc(-c4cccc5c4oc4ccccc45)n3)c2)cc1]

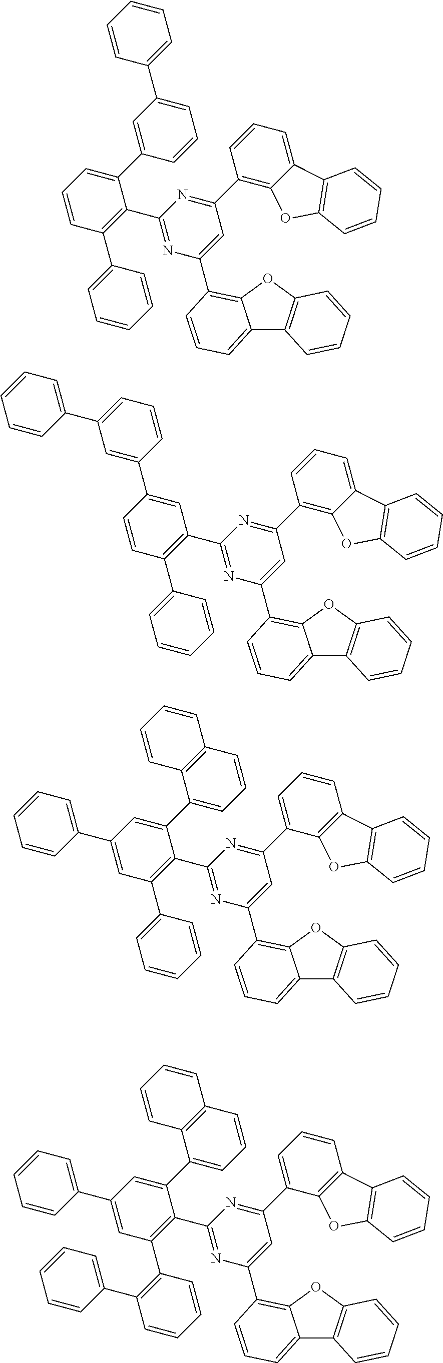 [SMILES: c1ccc(-c2cccc(-c3cccc(-c4ccccc4)c3-c3nc(-c4cccc5c4oc4ccccc45)cc(-c4cccc5c4oc4ccccc45)n3)c2)cc1 ; c1ccc(-c2cccc(-c3ccc(-c4ccccc4)c(-c4nc(-c5cccc6c5oc5ccccc56)cc(-c5cccc6c5oc5ccccc56)n4)c3)c2)cc1 ; c1ccc(-c2cc(-c3ccccc3)c(-c3nc(-c4cccc5c4oc4ccccc45)cc(-c4cccc5c4oc4ccccc45)n3)c(-c3cccc4ccccc34)c2)cc1 ; c1ccc(-c2cc(-c3ccccc3-c3ccccc3)c(-c3nc(-c4cccc5c4oc4ccccc45)cc(-c4cccc5c4oc4ccccc45)n3)c(-c3cccc4ccccc34)c2)cc1]

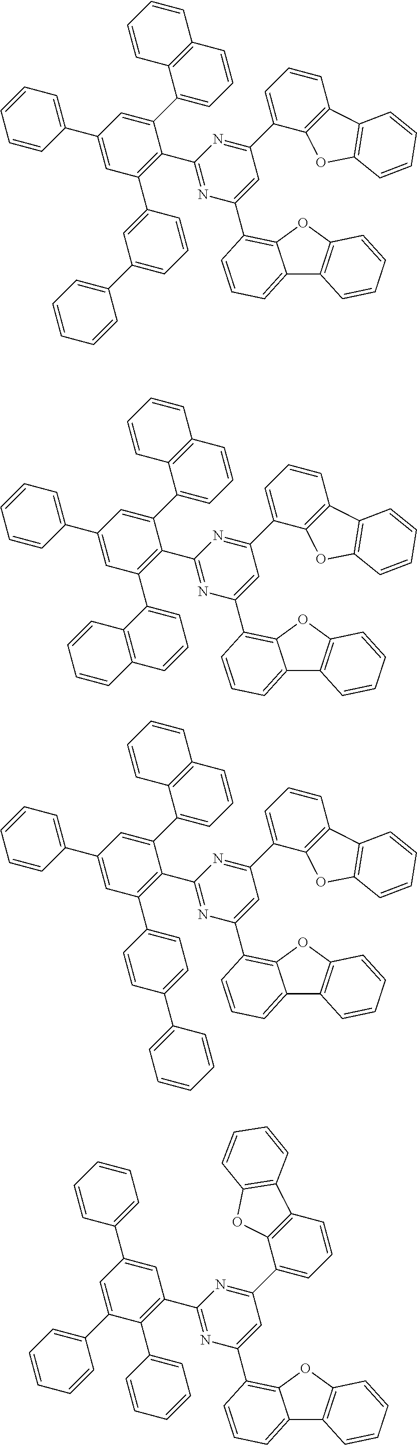 [SMILES: c1ccc(-c2cccc(-c3cc(-c4ccccc4)cc(-c4cccc5ccccc45)c3-c3nc(-c4cccc5c4oc4ccccc45)cc(-c4cccc5c4oc4ccccc45)n3)c2)cc1 ; c1ccc(-c2cc(-c3cccc4ccccc34)c(-c3nc(-c4cccc5c4oc4ccccc45)cc(-c4cccc5c4oc4ccccc45)n3)c(-c3cccc4ccccc34)c2)cc1 ; c1ccc(-c2ccc(-c3cc(-c4ccccc4)cc(-c4cccc5ccccc45)c3-c3nc(-c4cccc5c4oc4ccccc45)cc(-c4cccc5c4oc4ccccc45)n3)cc2)cc1 ; c1ccc(-c2cc(-c3ccccc3)c(-c3ccccc3)c(-c3nc(-c4cccc5c4oc4ccccc45)cc(-c4cccc5c4oc4ccccc45)n3)c2)cc1]

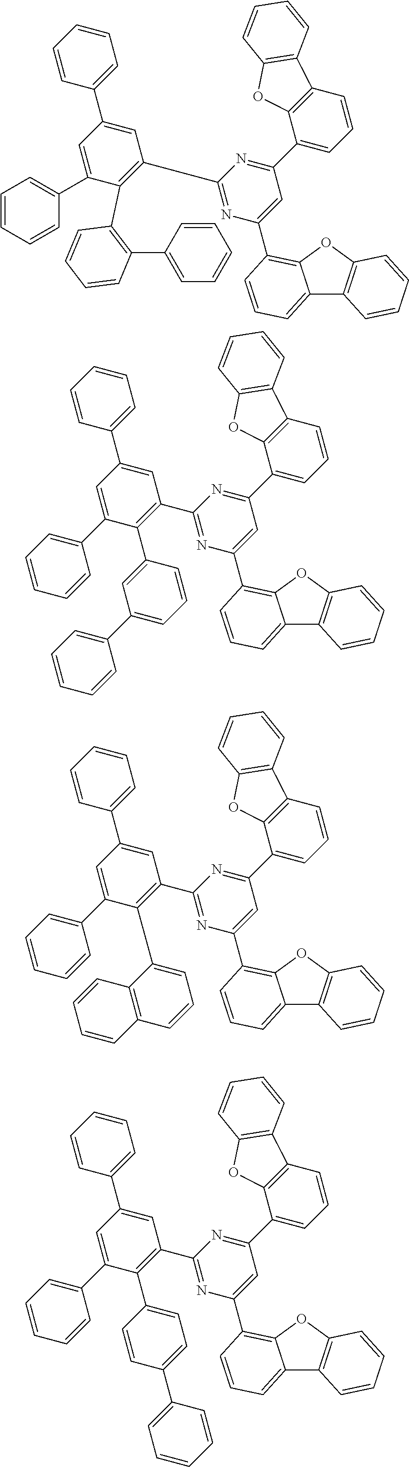

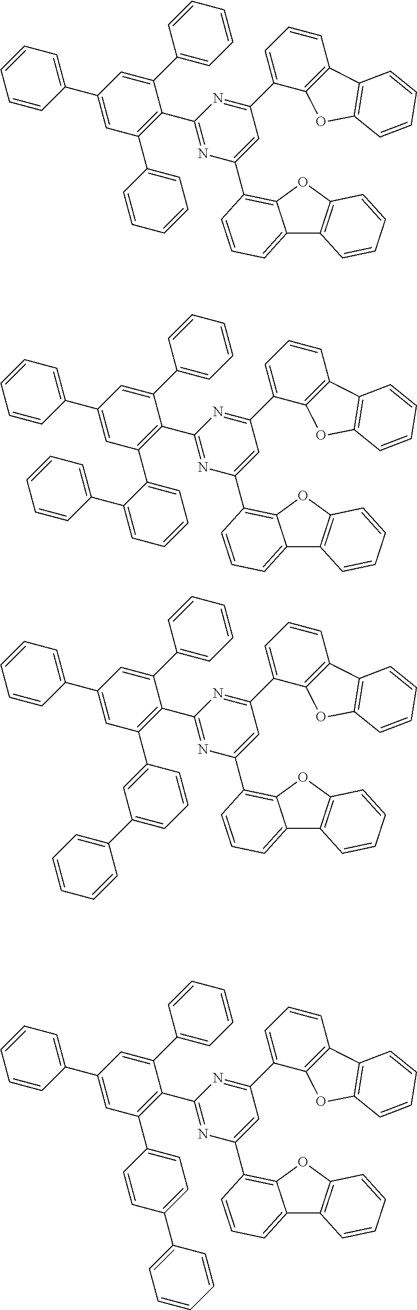

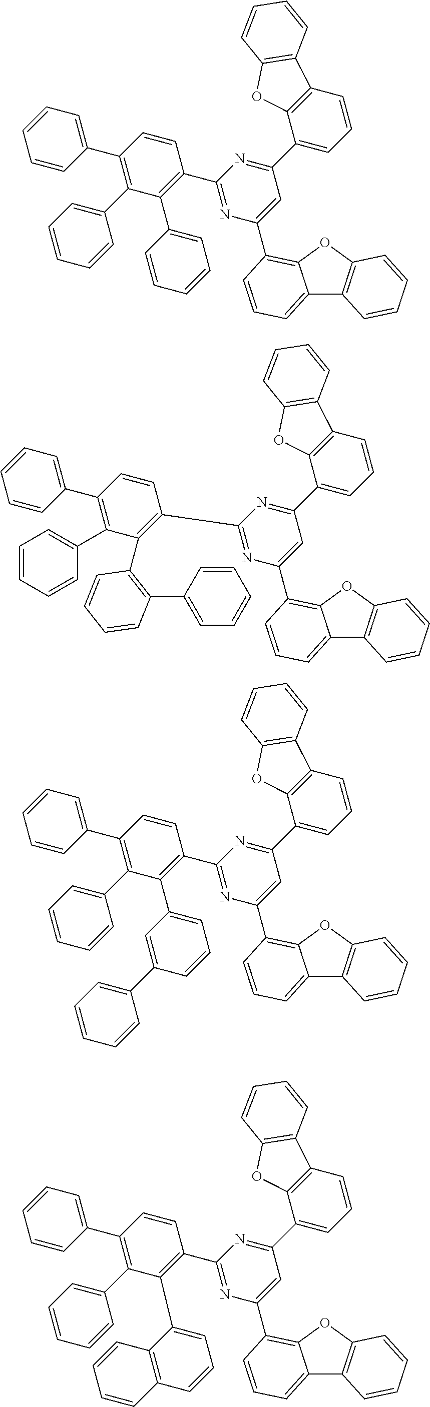

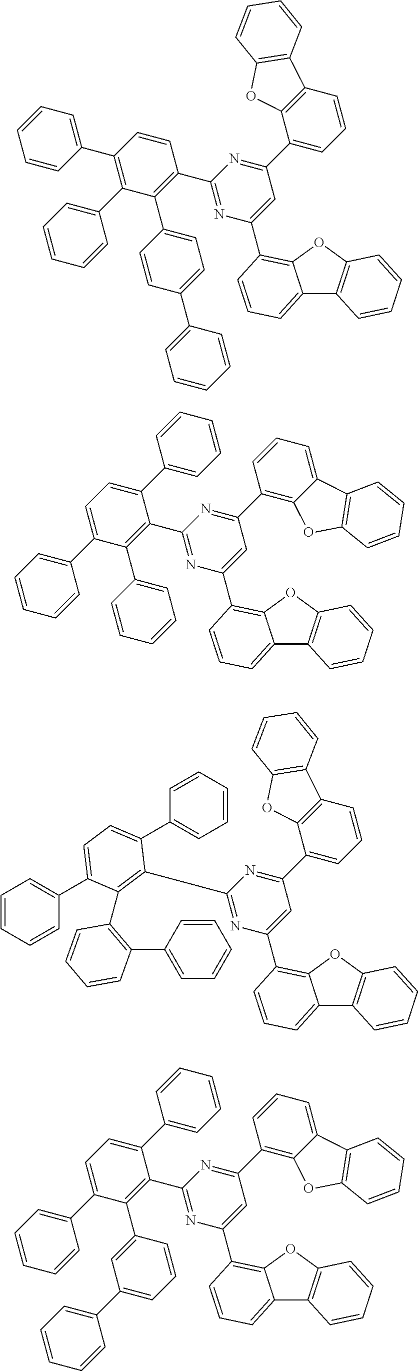

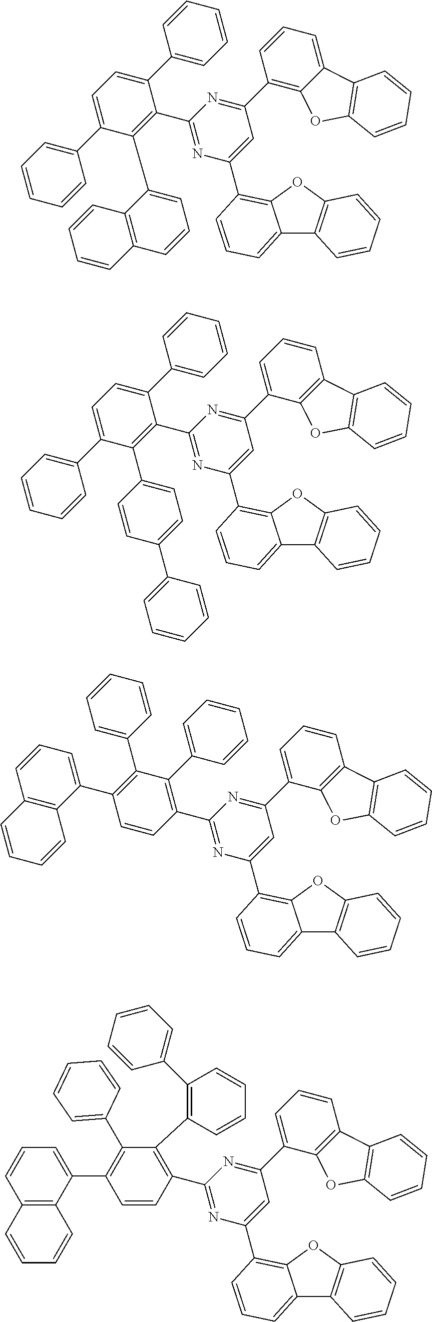 [SMILES: c1ccc(-c2ccc(-c3ccccc3)c(-c3cccc4ccccc34)c2-c2nc(-c3cccc4c3oc3ccccc34)cc(-c3cccc4c3oc3ccccc34)n2)cc1 ; c1ccc(-c2ccc(-c3c(-c4ccccc4)ccc(-c4ccccc4)c3-c3nc(-c4cccc5c4oc4ccccc45)cc(-c4cccc5c4oc4ccccc45)n3)cc2)cc1 ; c1ccc(-c2c(-c3nc(-c4cccc5c4oc4ccccc45)cc(-c4cccc5c4oc4ccccc45)n3)ccc(-c3cccc4ccccc34)c2-c2ccccc2)cc1 ; c1ccc(-c2ccccc2-c2c(-c3nc(-c4cccc5c4oc4ccccc45)cc(-c4cccc5c4oc4ccccc45)n3)ccc(-c3cccc4ccccc34)c2-c2ccccc2)cc1]

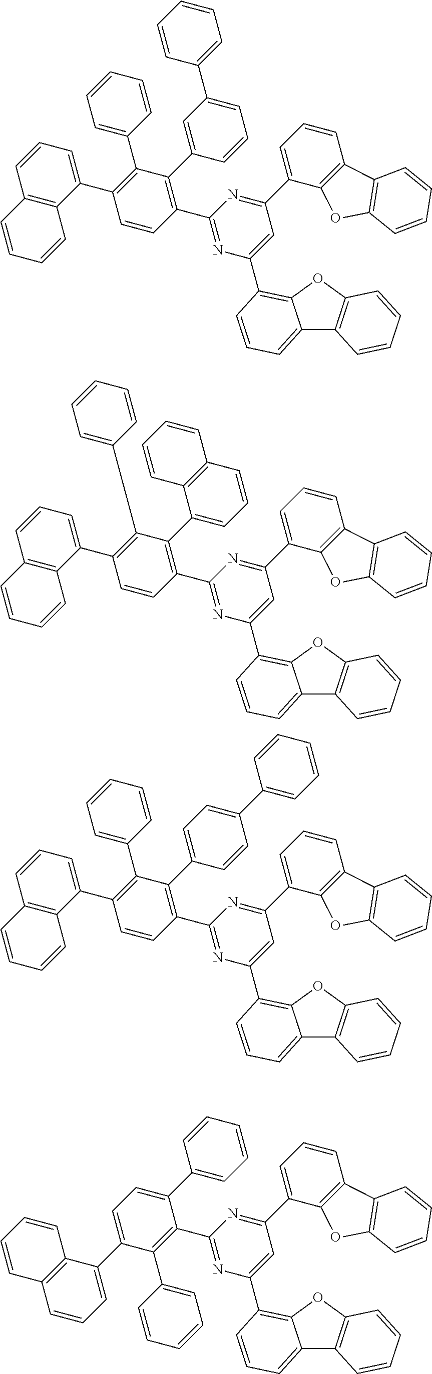

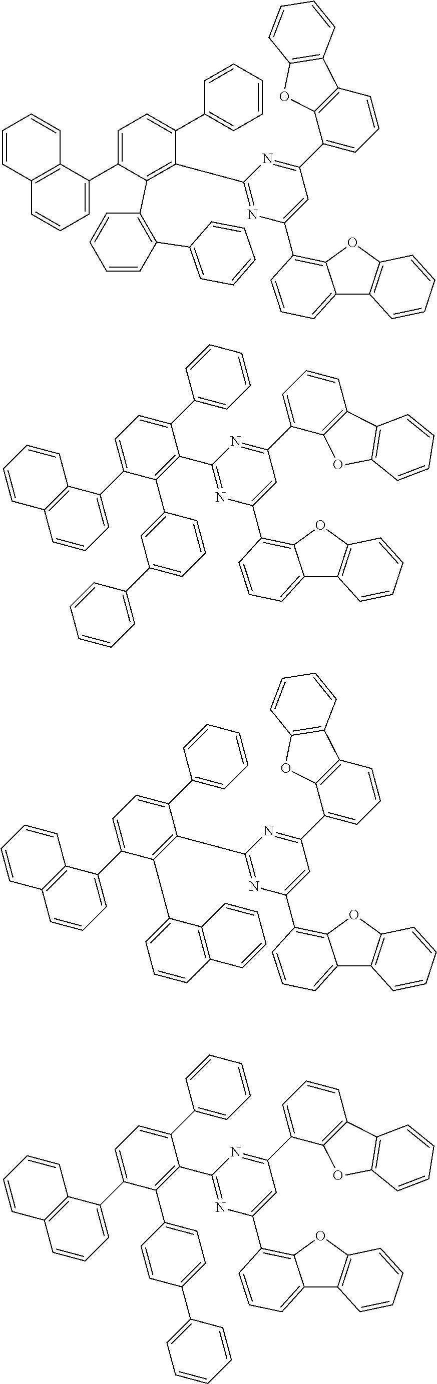 [SMILES: c1ccc(-c2ccccc2-c2c(-c3cccc4ccccc34)ccc(-c3ccccc3)c2-c2nc(-c3cccc4c3oc3ccccc34)cc(-c3cccc4c3oc3ccccc34)n2)cc1 ; c1ccc(-c2cccc(-c3c(-c4cccc5ccccc45)ccc(-c4ccccc4)c3-c3nc(-c4cccc5c4oc4ccccc45)cc(-c4cccc5c4oc4ccccc45)n3)c2)cc1 ; c1ccc(-c2ccc(-c3cccc4ccccc34)c(-c3cccc4ccccc34)c2-c2nc(-c3cccc4c3oc3ccccc34)cc(-c3cccc4c3oc3ccccc34)n2)cc1 ; c1ccc(-c2ccc(-c3c(-c4cccc5ccccc45)ccc(-c4ccccc4)c3-c3nc(-c4cccc5c4oc4ccccc45)cc(-c4cccc5c4oc4ccccc45)n3)cc2)cc1]

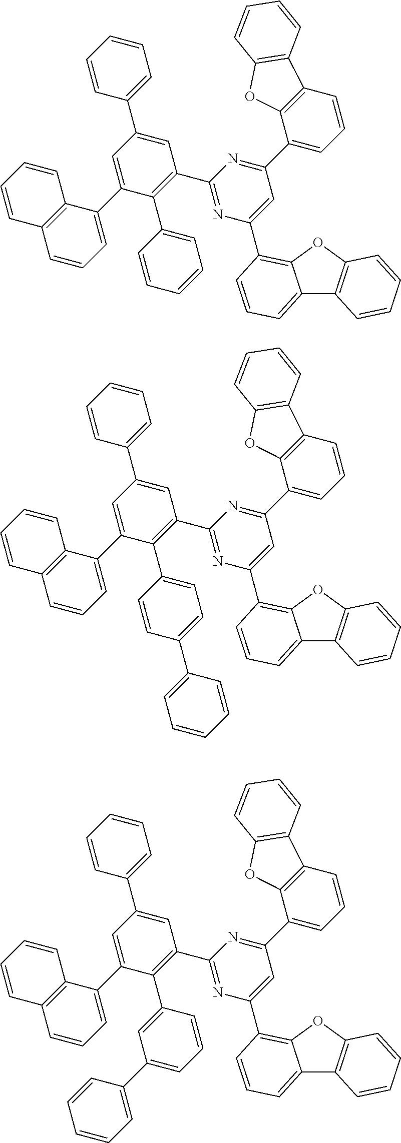

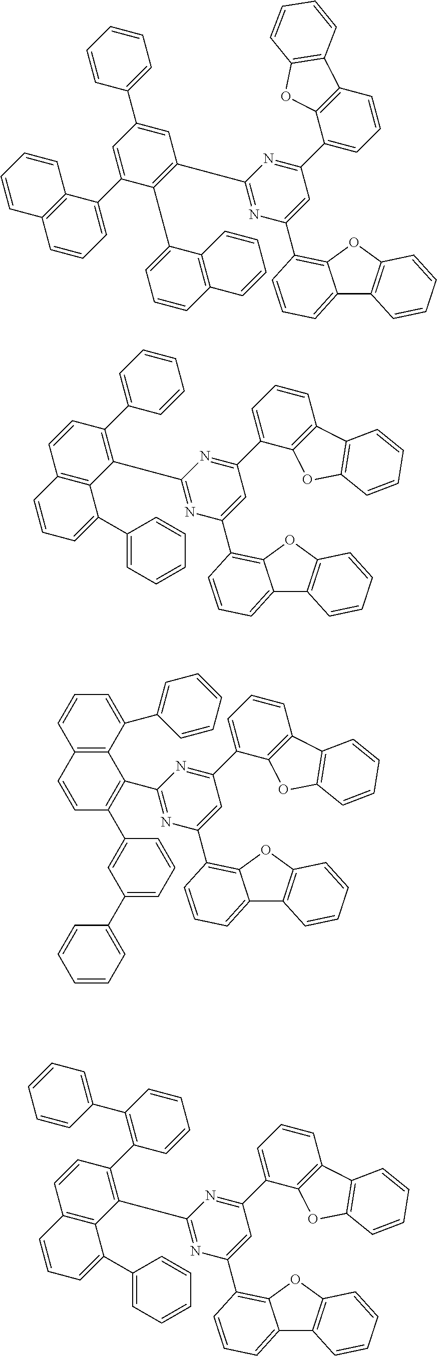 [SMILES: c1ccc(-c2cc(-c3nc(-c4cccc5c4oc4ccccc45)cc(-c4cccc5c4oc4ccccc45)n3)c(-c3cccc4ccccc34)c(-c3cccc4ccccc34)c2)cc1 ; c1ccc(-c2ccc3cccc(-c4ccccc4)c3c2-c2nc(-c3cccc4c3oc3ccccc34)cc(-c3cccc4c3oc3ccccc34)n2)cc1 ; c1ccc(-c2cccc(-c3ccc4cccc(-c5ccccc5)c4c3-c3nc(-c4cccc5c4oc4ccccc45)cc(-c4cccc5c4oc4ccccc45)n3)c2)cc1 ; c1ccc(-c2ccccc2-c2ccc3cccc(-c4ccccc4)c3c2-c2nc(-c3cccc4c3oc3ccccc34)cc(-c3cccc4c3oc3ccccc34)n2)cc1]

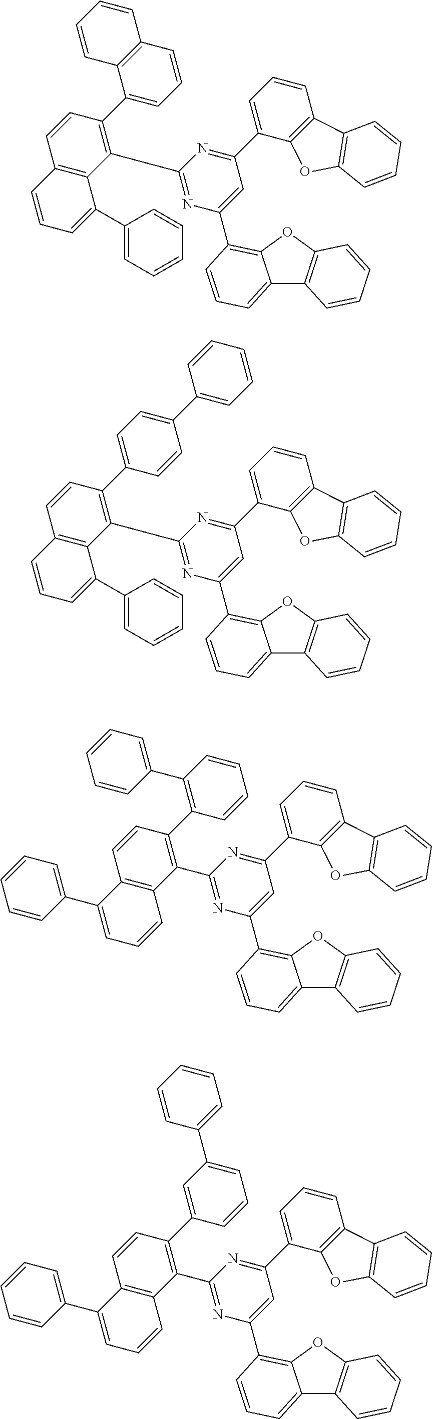

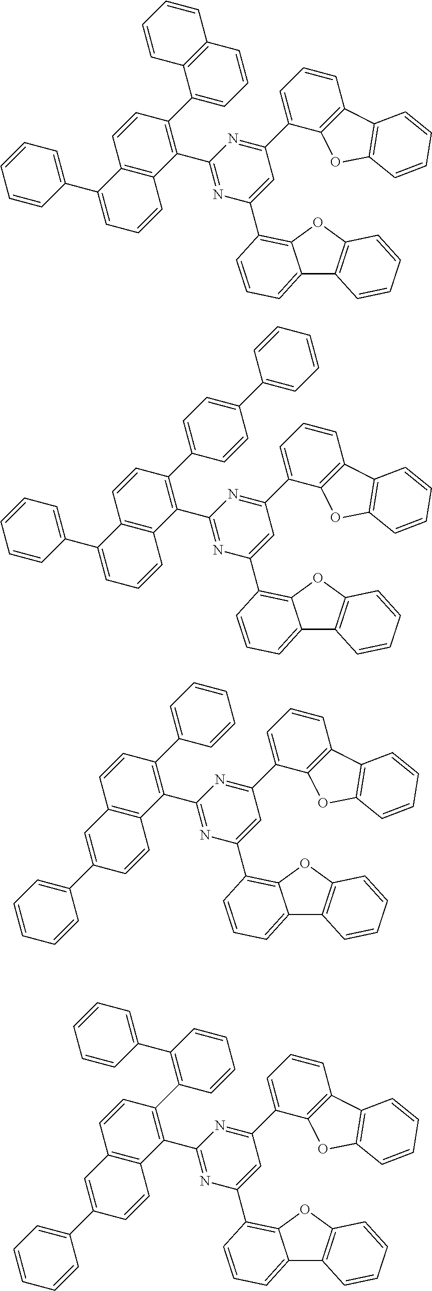

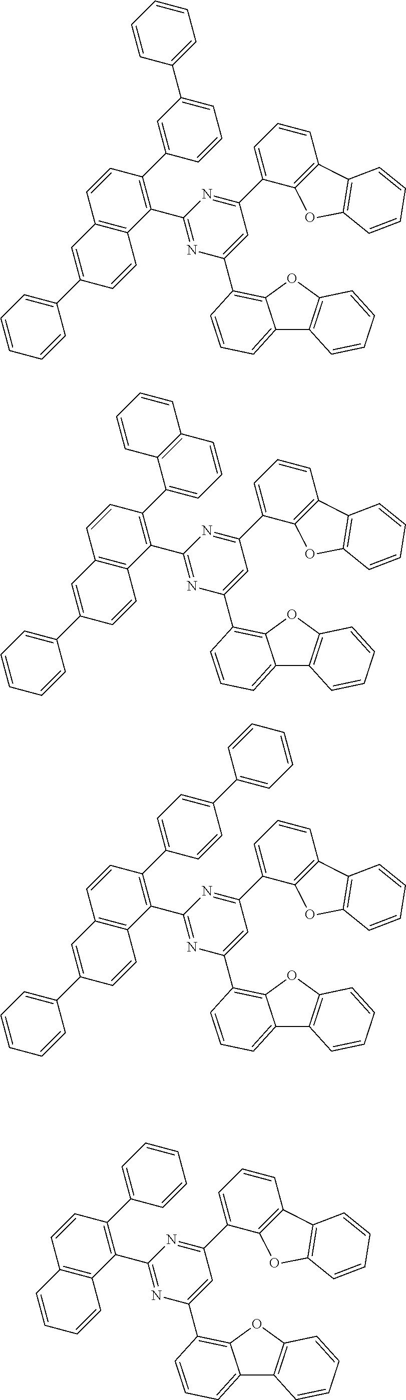

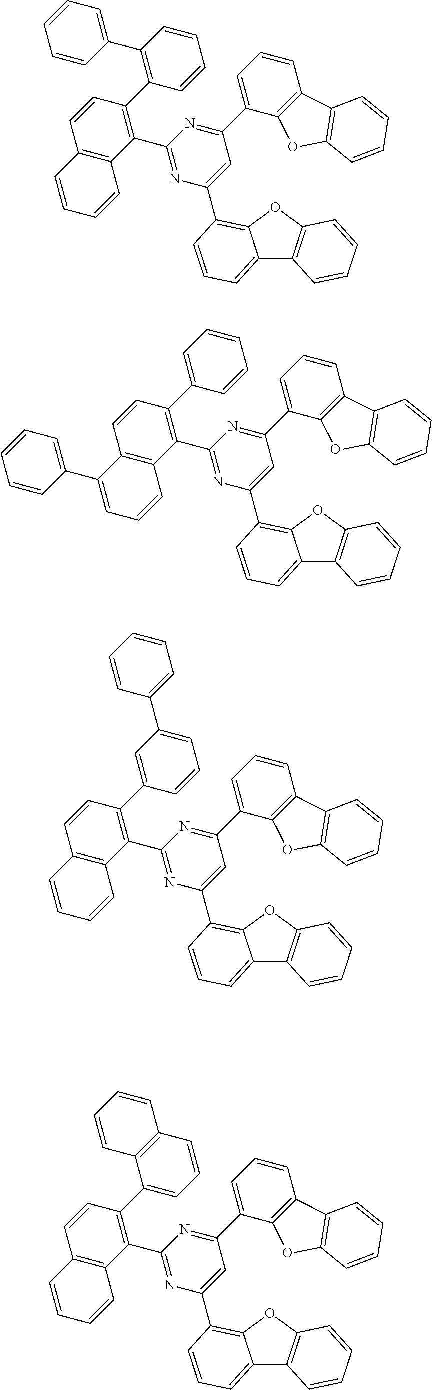 [SMILES: c1ccc(-c2ccccc2-c2ccc3ccccc3c2-c2nc(-c3cccc4c3oc3ccccc34)cc(-c3cccc4c3oc3ccccc34)n2)cc1 ; c1ccc(-c2ccc3c(-c4ccccc4)cccc3c2-c2nc(-c3cccc4c3oc3ccccc34)cc(-c3cccc4c3oc3ccccc34)n2)cc1 ; c1ccc(-c2cccc(-c3ccc4ccccc4c3-c3nc(-c4cccc5c4oc4ccccc45)cc(-c4cccc5c4oc4ccccc45)n3)c2)cc1 ; c1ccc2c(-c3ccc4ccccc4c3-c3nc(-c4cccc5c4oc4ccccc45)cc(-c4cccc5c4oc4ccccc45)n3)cccc2c1]

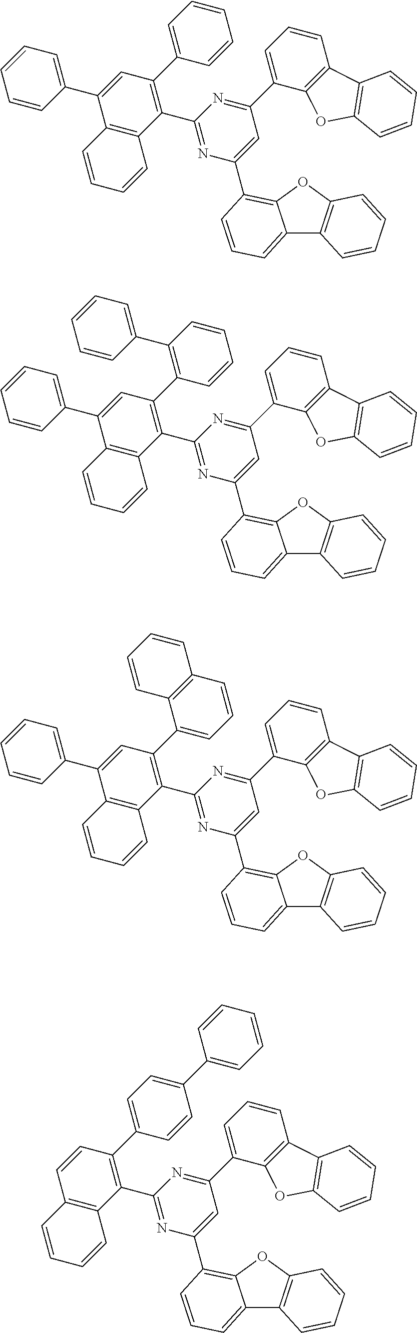 [SMILES: c1ccc(-c2cc(-c3ccccc3)c3ccccc3c2-c2nc(-c3cccc4c3oc3ccccc34)cc(-c3cccc4c3oc3ccccc34)n2)cc1 ; c1ccc(-c2ccccc2-c2cc(-c3ccccc3)c3ccccc3c2-c2nc(-c3cccc4c3oc3ccccc34)cc(-c3cccc4c3oc3ccccc34)n2)cc1 ; c1ccc(-c2cc(-c3cccc4ccccc34)c(-c3nc(-c4cccc5c4oc4ccccc45)cc(-c4cccc5c4oc4ccccc45)n3)c3ccccc23)cc1 ; c1ccc(-c2ccc(-c3ccc4ccccc4c3-c3nc(-c4cccc5c4oc4ccccc45)cc(-c4cccc5c4oc4ccccc45)n3)cc2)cc1]

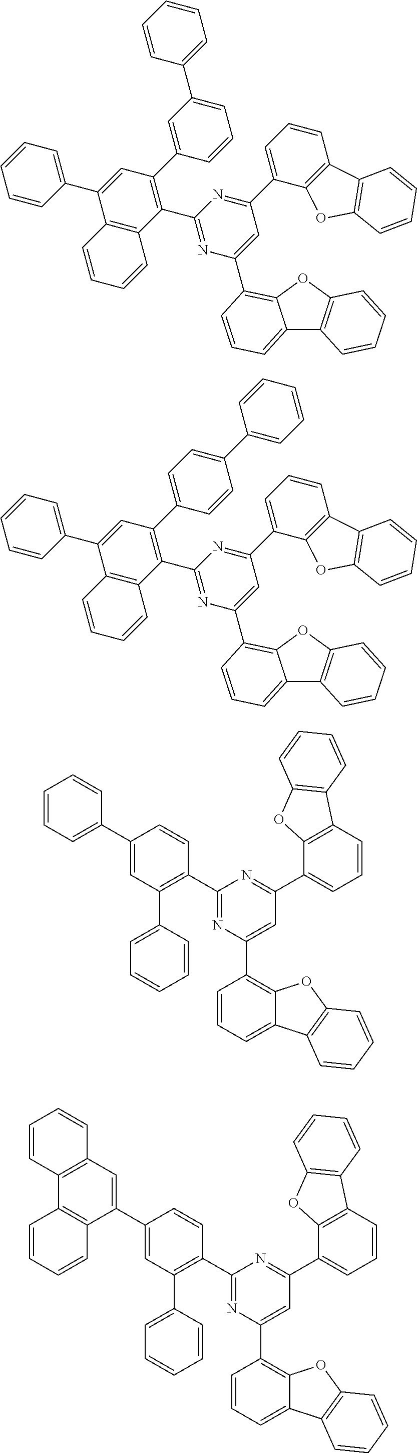

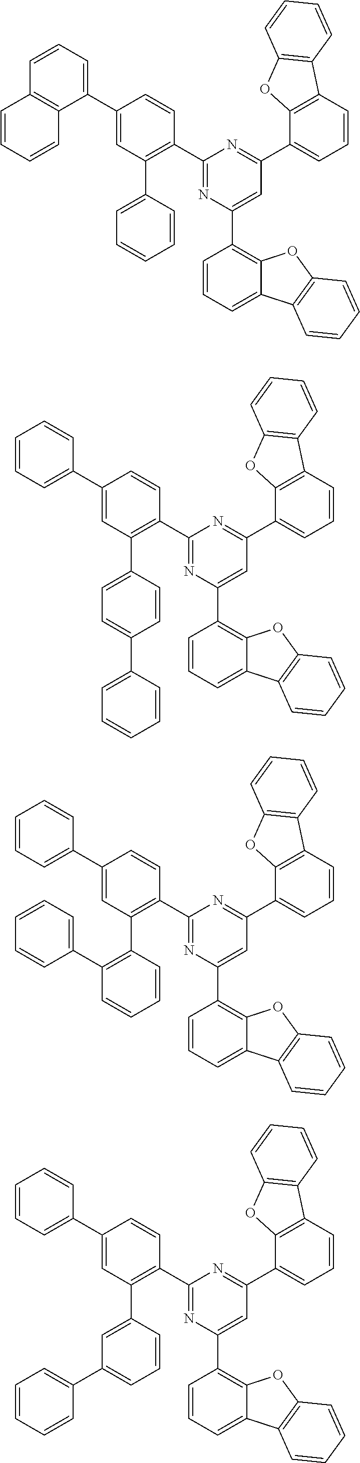

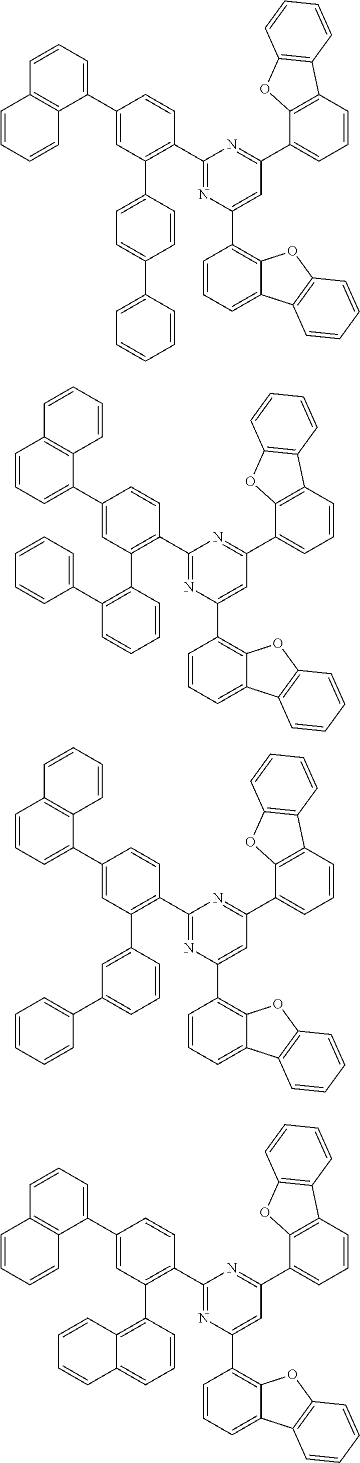

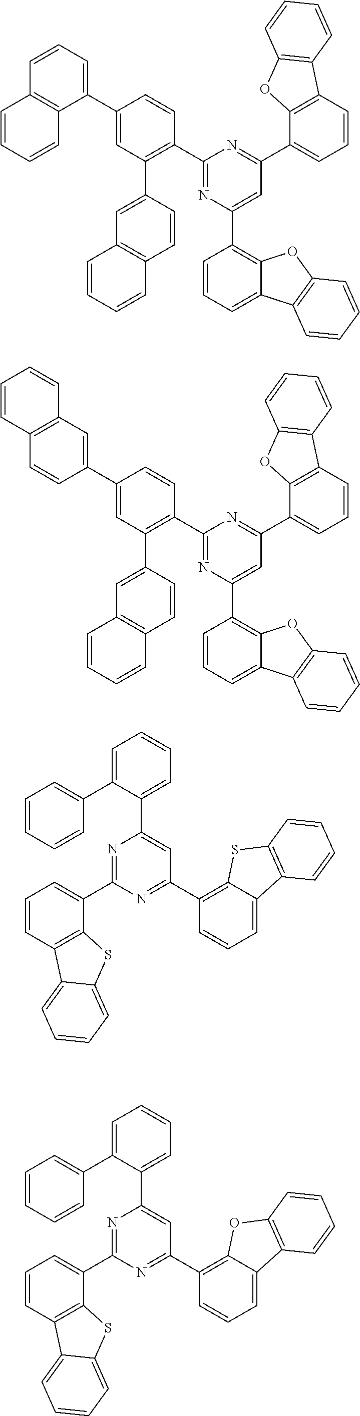

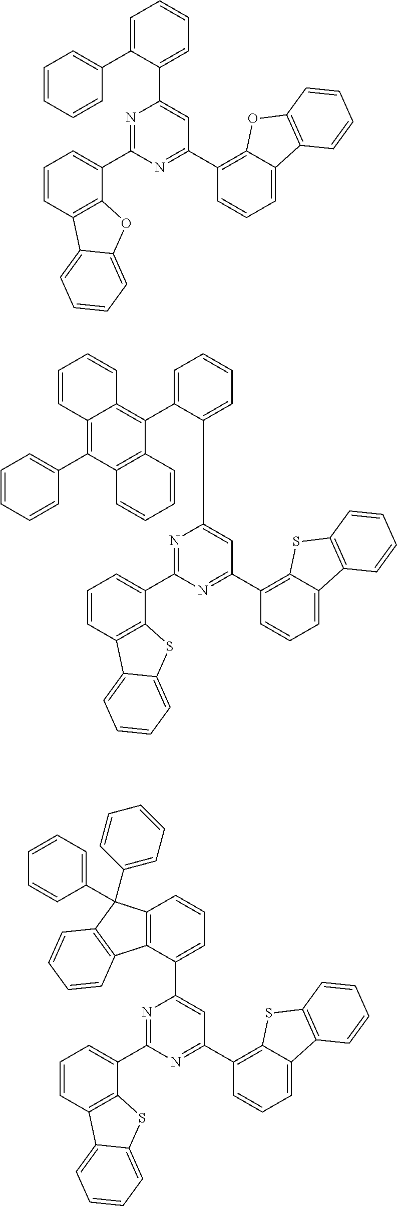 [SMILES: c1ccc(-c2ccccc2-c2cc(-c3cccc4c3oc3ccccc34)nc(-c3cccc4c3oc3ccccc34)n2)cc1 ; c1ccc(-c2c3ccccc3c(-c3ccccc3-c3cc(-c4cccc5c4sc4ccccc45)nc(-c4cccc5c4sc4ccccc45)n3)c3ccccc23)cc1 ; c1ccc(C2(c3ccccc3)c3ccccc3-c3c(-c4cc(-c5cccc6c5sc5ccccc56)nc(-c5cccc6c5sc5ccccc56)n4)cccc32)cc1]

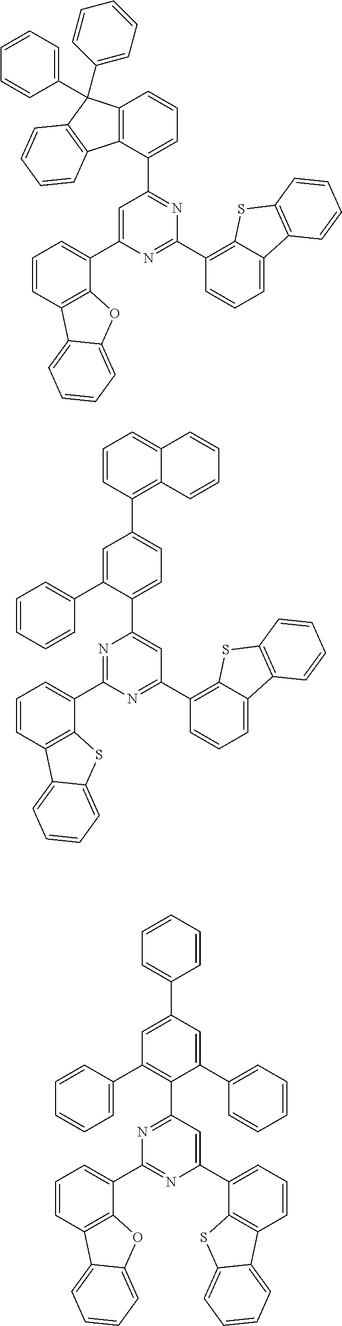 [SMILES: c1ccc(C2(c3ccccc3)c3ccccc3-c3c(-c4cc(-c5cccc6c5oc5ccccc56)nc(-c5cccc6c5sc5ccccc56)n4)cccc32)cc1 ; c1ccc(-c2cc(-c3cccc4ccccc34)ccc2-c2cc(-c3cccc4c3sc3ccccc34)nc(-c3cccc4c3sc3ccccc34)n2)cc1 ; c1ccc(-c2cc(-c3ccccc3)c(-c3cc(-c4cccc5c4sc4ccccc45)nc(-c4cccc5c4oc4ccccc45)n3)c(-c3ccccc3)c2)cc1]

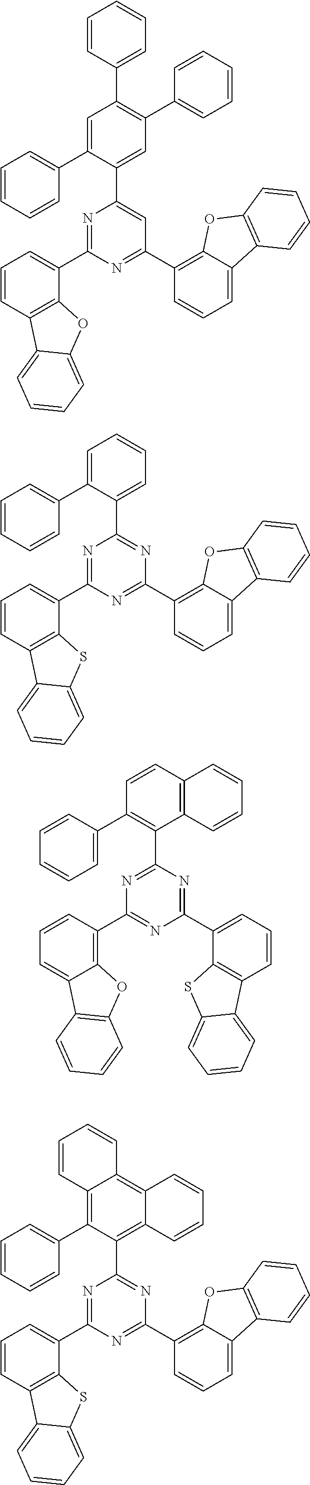

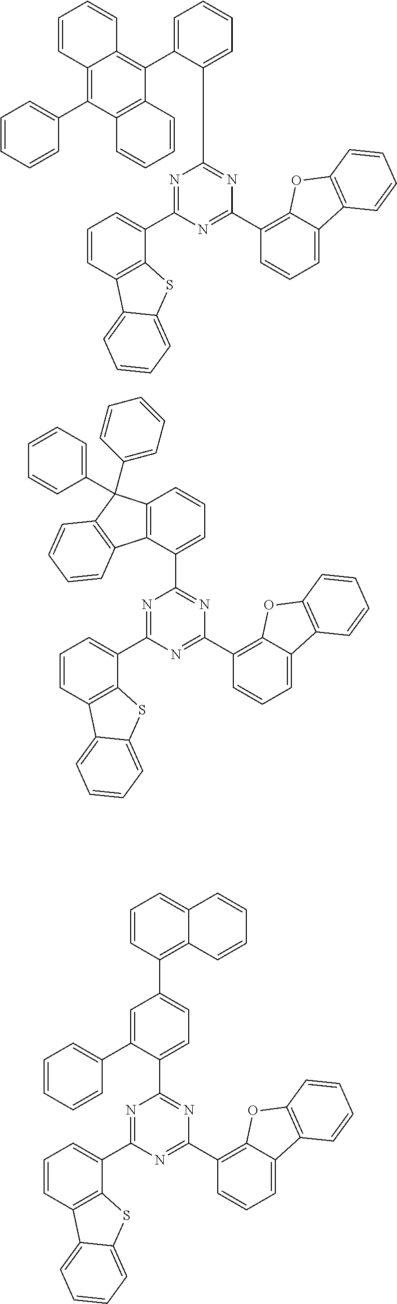

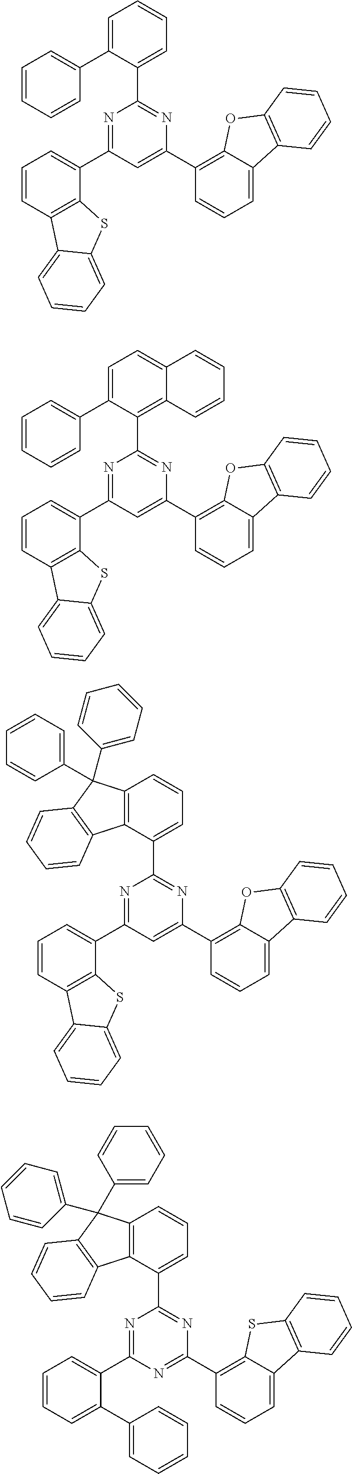

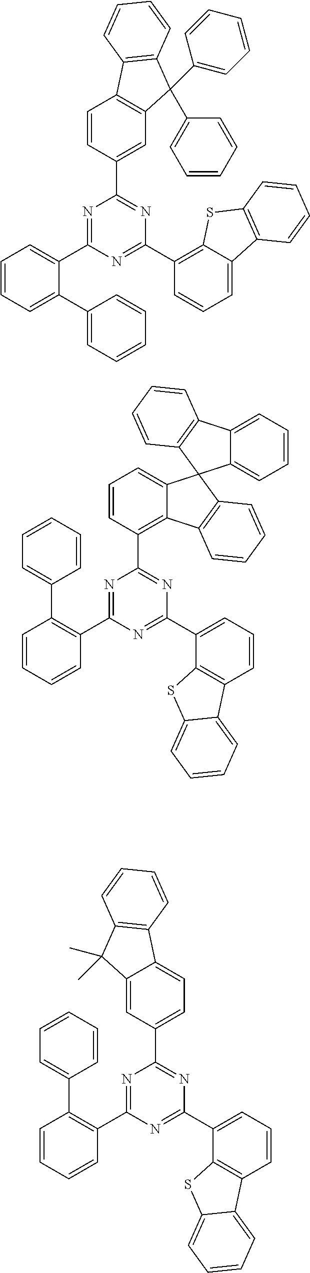

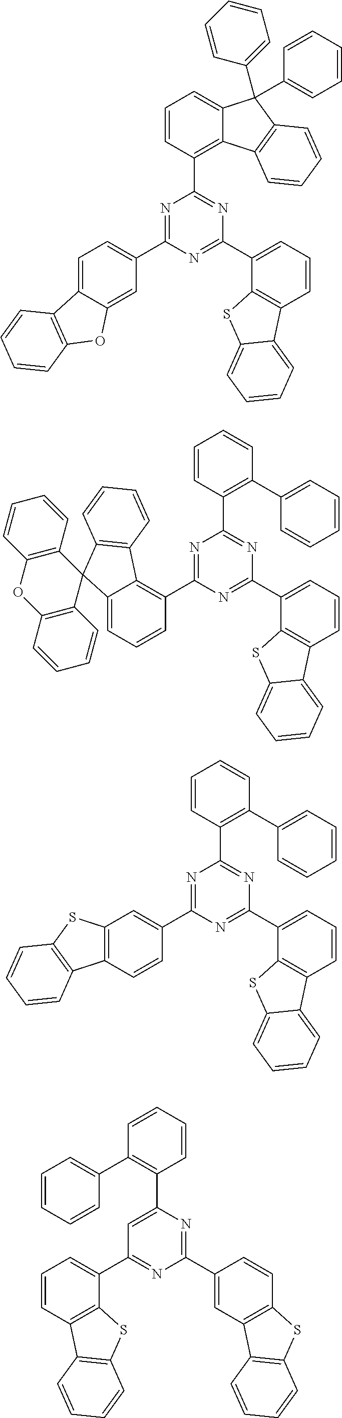

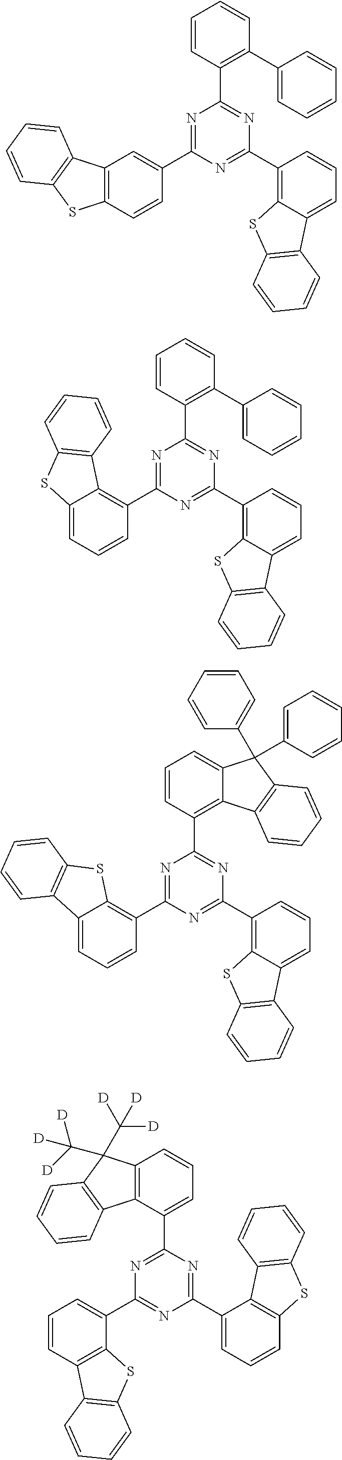

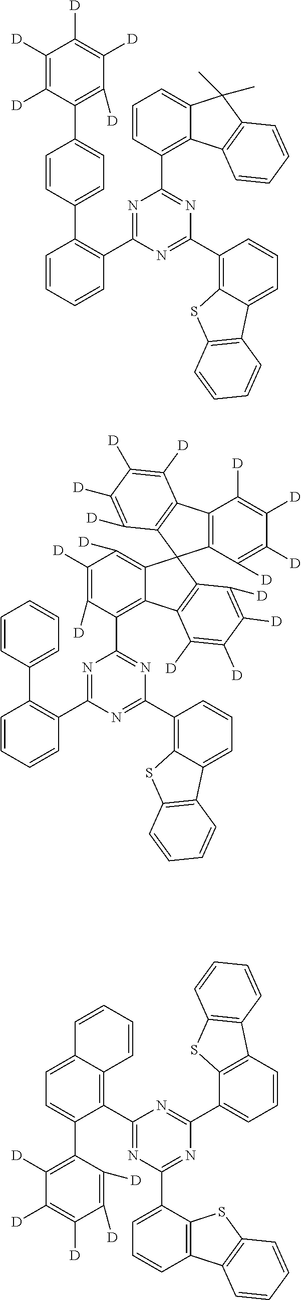 [SMILES: [2H]c1c([2H])c([2H])c(-c2ccc(-c3ccccc3-c3nc(-c4cccc5c4-c4ccccc4C5(C)C)nc(-c4cccc5c4sc4ccccc45)n3)cc2)c([2H])c1[2H] ; [2H]c1c([2H])c([2H])c2c(c1[2H])-c1c([2H])c([2H])c([2H])c([2H])c1C21c2c([2H])c([2H])c([2H])c([2H])c2-c2c(-c3nc(-c4ccccc4-c4ccccc4)nc(-c4cccc5c4sc4ccccc45)n3)c([2H])c([2H])c([2H])c21 ; [2H]c1c([2H])c([2H])c(-c2ccc3ccccc3c2-c2nc(-c3cccc4c3sc3ccccc34)nc(-c3cccc4c3sc3ccccc34)n2)c([2H])c1[2H]]

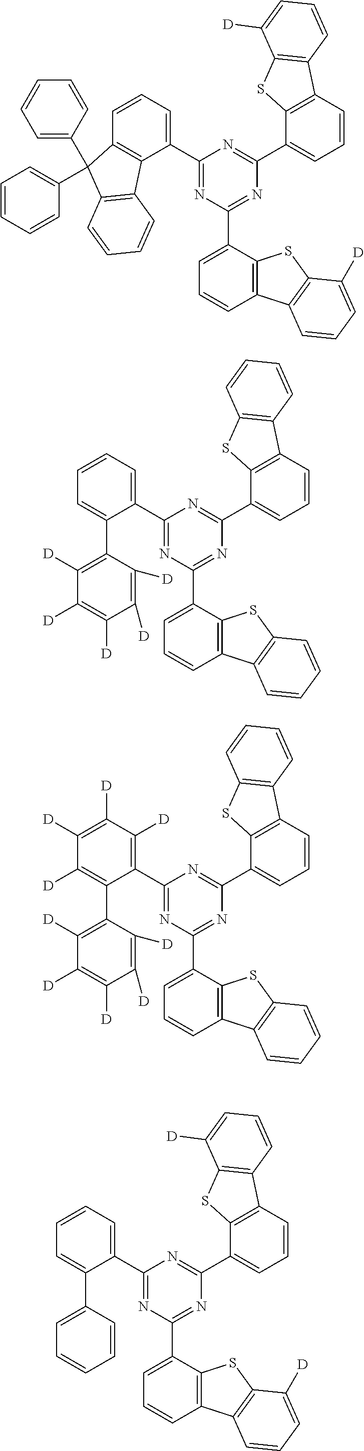

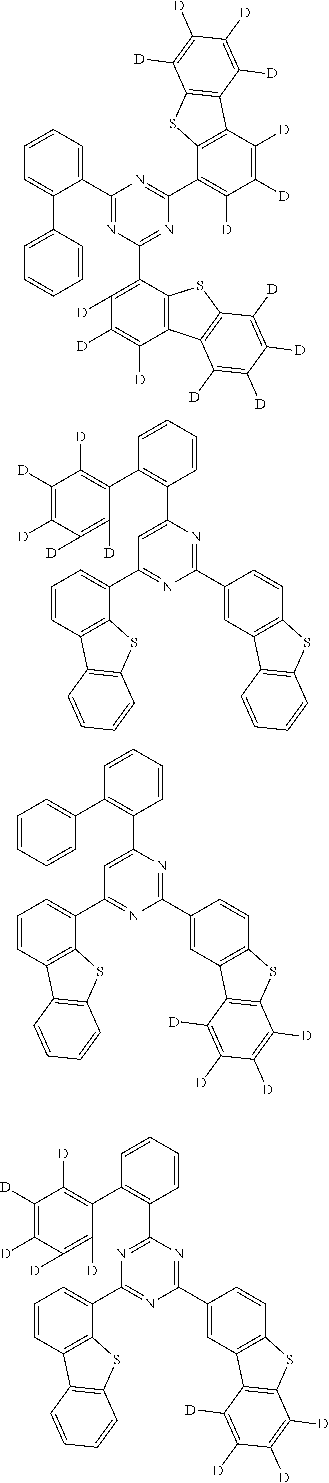

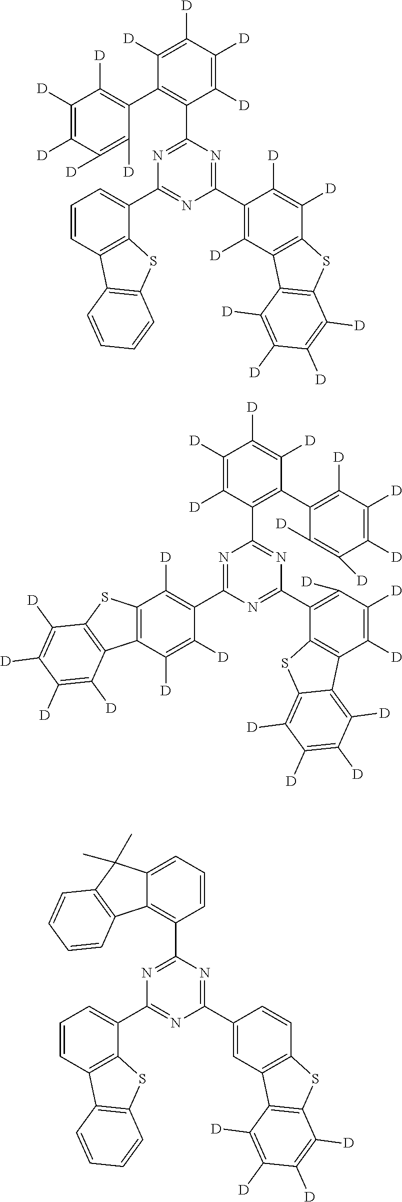

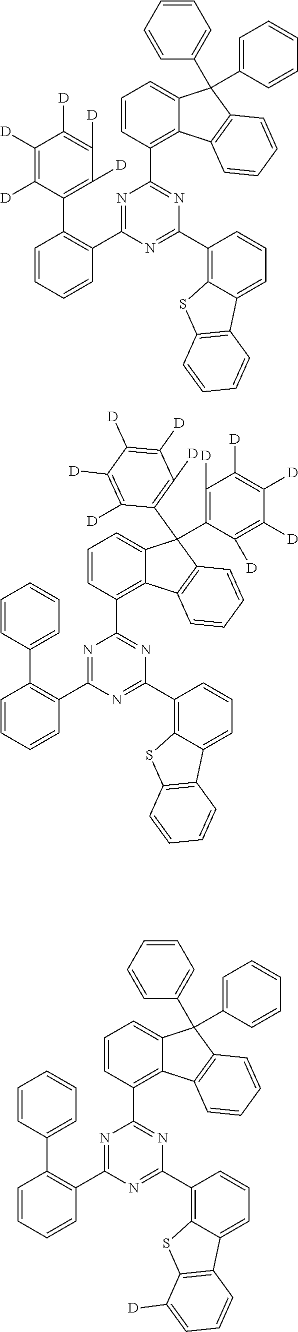

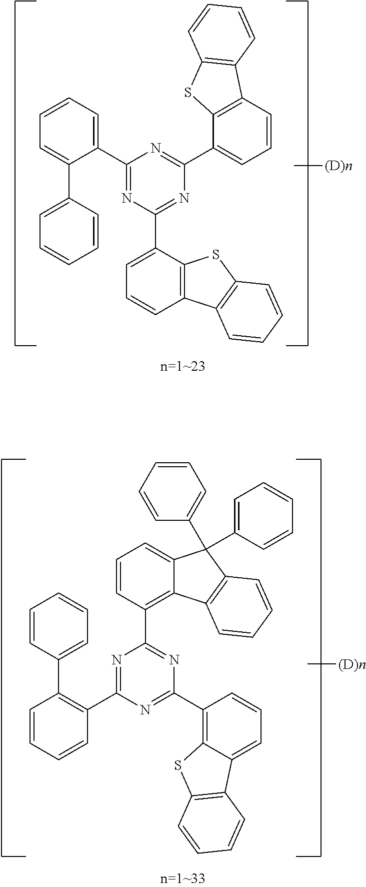

Known alternative reaction or raw materials according to an intended product are used in copying the synthesis in Examples described later, whereby the compound represented by the formula (A1) can be synthesized.

### [Electron-Transporting Material for Organic EL Device]

The compound represented by the formula (A1) according to an aspect of the invention is useful as a material for an organic EL device, and particularly useful as an electron-transporting material or a phosphorescent host material.

The electron-transporting material for an organic EL device according to an aspect of the invention comprises the compound represented by the formula (A1).

### [Organic Electroluminescence Device]

The organic electroluminescence device according to an aspect of the invention comprises an anode, an organic layer, and a cathode in this order, wherein
the organic layer comprises the compound represented by the formula (A1).

When the organic EL device comprises a plurality of organic layers, the compound represented by the formula (A1) may be contained any layer among the plurality of the organic layers. The types of organic layers will be described later.

Also, the organic electroluminescence device according to an aspect of the invention comprises an anode, an emitting layer, an electron-transporting region, and a cathode, in this order, wherein
the electron transporting region comprises the compound represented by the formula (A1).

In one embodiment, the electron-transporting region comprises a first electron-transporting layer, and a second electron-transporting layer, and the emitting layer, the first electron-transporting layer, the second electron-transporting layer and the cathode in this order, and
at least one of the first electron-transporting layer and the second electron-transporting layer comprises the compound represented by the formula (A1).

In one embodiment, the electron-transporting region comprises a first electron-transporting layer, and a second electron-transporting layer, and the emitting layer, the first electron-transporting layer, the second electron-transporting layer and the cathode in this order, and
the second electron-transporting layer comprises the compound represented by the formula (A1).

By including the compound represented by the formula (A1) in one or both of the first electron-transporting layer and the second electron-transporting layer, an organic EL device with high luminous efficiency can be obtained.

In one embodiment of an organic EL device, a compound represented by the formula (A1) has at least one deuterium atom.

In addition, a compound represented by the formula (A1) may be a mixture of a compound represented by the formula (A1) in which all hydrogen atoms in the compound are protium atoms (hereinafter referred to as “protium compound (A1)”) and a compound represented by the formula (A1) in which at least one of hydrogen atom in the compound is a deuterium atom (hereinafter referred to as “deuterium compound (A1)”).

However, the protium compound (A1) may inevitably contain deuterium atoms at a proportion of the natural abundance ratio or less.

In one embodiment, the compound represented by the formula (A1) contained in either or both of the first electron-transporting layer and the second electron-transporting layer is preferably a compound represented by the formula (A1) in which all hydrogen atoms in the compound represented by the formula (A1) are protium atoms (protium compound (A1)) from the viewpoint of production costs.

Accordingly, one embodiment includes the organic EL device in which either or both of the first electron-transport layer and the second-electron transport layer contain a compound represented by the formula (A1) which substantially consist only of the protium compound (A1).

The “compound represented by the formula (A1) which substantially consist only of the protium compound (A1)” means that the content ratio of the protium compound (A1) relative to the total amount of the compound represented by the formula (A1) is 90 mol % or more, preferably 95 mol % or more, and more preferably 99 mol % or more (each including 100%).

Schematic configuration of organic EL device according to one aspect of the invention will be explained referring to the FIGURE.

Organic EL device 1 according to one aspect of the invention comprises: substrate 2; anode 3; organic thin film layer 4; emitting layer 5; organic thin film layer 6; and cathode 10 in this order. The organic thin film layer 4, which is positioned between the anode 3 and the emitting layer 5, functions as a hole-transporting region, and the organic thin film layer 6, which is positioned between the emitting layer 5 and the cathode 10, functions as an electron-transporting region.

The organic thin film layer 6 includes a first electron-transporting layer 6a which is positioned to the emitting layer 5 side and a second electron-transporting layer 6b which is positioned to the cathode 10 side.

One or both of the first electron-transporting layer 6a and the second electron-transporting layer 6b include the compound represented by the formula (A1). By including the compound represented by the formula (A1) in the first electron-transporting layer 6a or the second electron-transporting layer 6b, an organic EL device with improved luminous efficiency can be obtained.

In one embodiment, the emitting layer comprises the compound represented by the following formula (11).

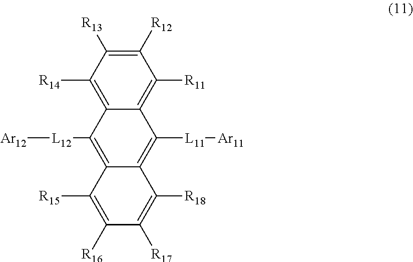

In the formula (11),
R₁₁ to R₁₈ are independently
   a hydrogen atom,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃)

### —O—(R₉₀₄)

### —S—(R₉₀₅)

—N(R₉₀₆)(R₉₀₇)
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
   a hydrogen atom,
   a substituted or unsubstituted alkyl group including 1 to 50 carbon atoms,
   a substituted or unsubstituted cycloalkyl group including 3 to 50 ring carbon atoms,
   a substituted or unsubstituted aryl group including 6 to 50 ring carbon atoms, or
   a substituted or unsubstituted monovalent heterocyclic group including 5 to 50 ring atoms;
when two or more of each of R₉₀₁ to R₉₀₇ are present, two or more of each of R₉₀₁ to R₉₀₇ may be the same as or different to each other;
adjacent two or more of R₁₁ to R₁₄, and adjacent two or more R₁₅ to R₁₈ do not form a ring by bonding with each other;
L₁₁ and L₁₂ are independently
   a single bond,
   a substituted or unsubstituted arylene group including 6 to 50 ring carbon atoms, or
   a substituted or unsubstituted divalent heterocyclic group including 5 to 50 ring atoms; and
Ar₁₁ and Ar₁₂ are independently
   a substituted or unsubstituted aryl group including 6 to 50 ring carbon atoms, or
   a substituted or unsubstituted monovalent heterocyclic group including 5 to 50 ring atoms.

Inclusion of the compound represented by the formula (11) in the emitting layer results in an organic EL device having a more increased luminous efficiency.

In one embodiment, the compound represented by the formula (11) is the compound represented by the following formula (12).

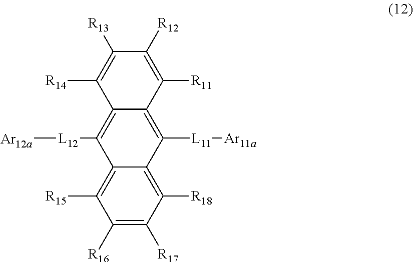

In the formula (12), R₁₁ to R₁₈, L₁₁ and L₁₂ are as defined in the formula (11);
at least one of Ar₁₁a and Ar₁₂ₐ is the monovalent group represented by the following formula (20):

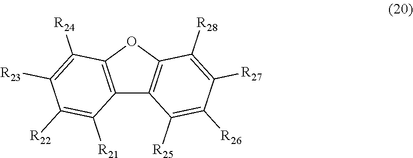

wherein in the formula (20),
one of R₂₁ to R₂₈ is a single bond bonding with L₁₁ or L₁₂;
R₂₁ to R₂₈ that are not single bond bonding with L₁₁ or L₁₂ are independently
   a hydrogen atom,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃)

### —O—(R₉₀₄)

### —S—(R₉₀₅)

—N(R₉₀₆)(R₉₀₇)
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are as defined in the formula (11); and
adjacent two or more of R₂₁ to R₂₈ that are not single bond bonding with L₁₁ or L₁₂ do not form a ring by bonding with each other;
Ar₁₁a or Ar₁₂ₐ that is not a monovalent group represented by the formula (20) is
   a substituted or unsubstituted aryl group including 6 to 50 ring carbon atoms, or
   a substituted or unsubstituted monovalent heterocyclic group including 5 to 50 ring atoms, other than the monovalent group represented by the formula (20).

In one embodiment, the compound represented by the formula (12) is the compound represented by the following formula (12-1).

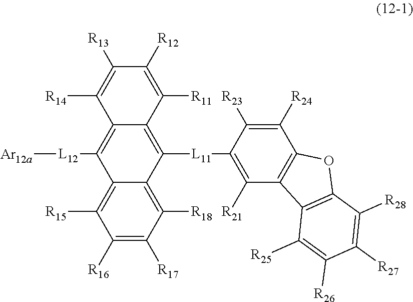

In the formula (12-1), R₁₁ to R₁₈, L₁₁ and L₁₂ are as defined in the formula (11);
Ar₁₂ₐ is a substituted or unsubstituted aryl group including 6 to 50 ring carbon atoms, or
   a substituted or unsubstituted monovalent heterocyclic group including 5 to 50 ring atoms, other than the monovalent group represented by the formula (20);
R₂₁ and R₂₃ to R₂₈ are independently a hydrogen atom,
   a substituted or unsubstituted alkyl group including 1 to 50 carbon atoms,
   a substituted or unsubstituted alkenyl group including 2 to 50 carbon atoms
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃)

### —O—(R₉₀₄)

### —S—(R₉₀₅)

—N(R₉₀₆)(R₉₀₇)
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms; and
R₉₀₁ to R₉₀₇ are as defined in the formula (11).

In one embodiment, the compound represented by the formula (11) is the compound represented by the following formula (13).

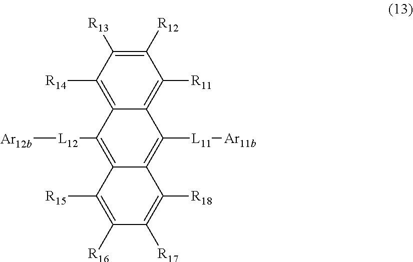

In the formula (13), R₁₁ to R₁₈, L₁₁ and L₁₂ are as defined in the formula (11);
Ar_{11b} and Ar_{12b} are independently
   a substituted or unsubstituted aryl group including 6 to 50 ring carbon atoms, that is constituted only with a benzene ring.

In one embodiment, the compound represented by the formula (13) is the compound represented by the following formula (13-1).

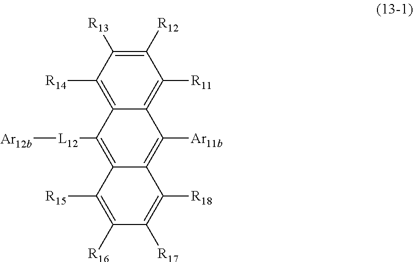

In the formula (13-1), R₁₁ to R₁₈ and L₁₂ are as defined in the formula (11); and
Ar_{11b} and Ar_{12b} are independently a substituted or unsubstituted aryl group including 6 to 50 ring carbon atoms, that are constituted only with a benzene ring.

Here, the aryl group “constituted only with a benzene ring” means that aryl groups including a ring other than the benzene ring are excluded. Specifically, a group derived from a fluorene ring which includes a 5-membered ring in addition to benzene rings, and the like are excluded.

The aryl group “constituted only of a benzene ring” includes a group composed of a monocycle of a benzene ring (namely, a phenyl group), a group in which two or more benzene rings are sequentially linked via a single bond (for example, a biphenylyl group, or the like), and a group formed by fusing benzene rings (for example, a naphthyl group, or the like).

The aryl group constituted only of a benzene ring may be substituted by an optional substituent.

In one embodiment, Ar_{11b} and Ar_{12b} are independently

a substituted or unsubstituted phenyl group,
a substituted or unsubstituted naphthyl group,
a substituted or unsubstituted biphenylyl group,
a substituted or unsubstituted terphenylyl group,
a substituted or unsubstituted anthryl group, or
a substituted or unsubstituted phenanthryl group.

In one embodiment, the compound represented by the formula (11) is a compound represented by the following formula (14).

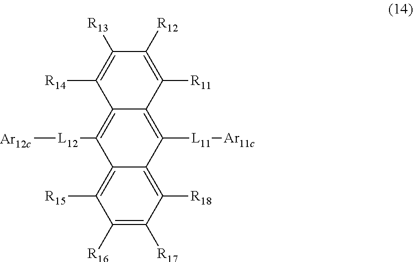

In the formula (14), R₁₁ to R₁₈, L₁₁ and L₁₂ are as defined in the formula (11);
at least one of Ar₁₁c and Ar₁₂c is a monovalent group represented by the following formula (30):

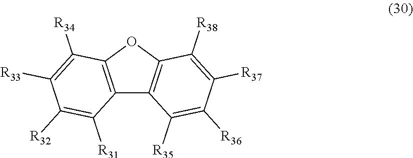

wherein in the formula (30),
adjacent two of R₃₁ to R₃₄, or adjacent two of R₃₅ to R₃₈ form an unsaturated ring represented by the following formula (40) by bonding with each other:

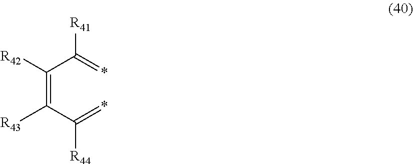

wherein in the formula (40),
two *'s are bonding positions with the adjacent two of R₃₁ to R₃₄, or the adjacent two of R₃₅ to R₃₈;
adjacent two or more of R₃₁ to R₃₈ which do not form the unsaturated ring represented by the formula (40), and R₄₁ to R₄₄ do not form a ring by bonding with each other;
one of R₃₁ to R₃₈ which do not form the unsaturated ring represented by the formula (40), and R₄₁ to R₄₄ is a single bond bonding with L₁₁ or L₁₂;
R₃₁ to R₃₈ which do not form the unsaturated ring represented by the formula (40) and are not the single bond bonding with L₁₁ or L₁₂, and R₄₁ to R₄₄ which are not the single bond bonding with L₁₁ or L₁₂ are independently
   a hydrogen atom,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃),

### —O—(R₉₀₄),

### —S—(R₉₀₅),

—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms; and
R₉₀₁ to R₉₀₇ are as defined in the formula (11).

In one embodiment, the monovalent group represented by the formula (30) is selected from monovalent groups represented by any of the following formulas (30A) to (30C).

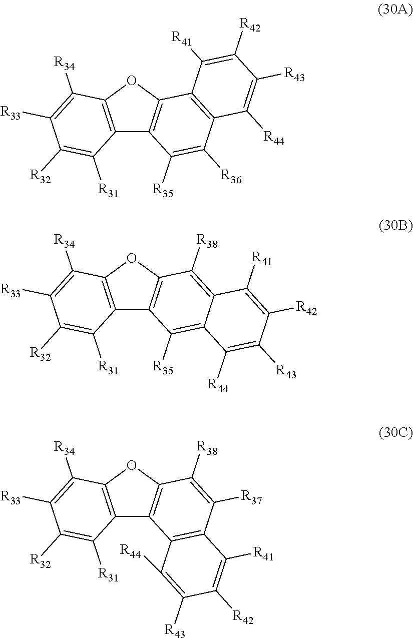

In the formulas (30A) to (30C), R₃₁ to R₃₈ and R₄₁ to R₄₄ are as defined in the formula (14).

In one embodiment, the compound represented by the formula (11) is a compound represented by the following formula (15).

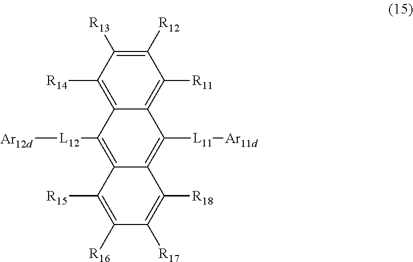

In the formula (15), R₁₁ to R₁₈, L₁₁ and L₁₂ are as defined in the formula (11);
at least one of Ar_{11d} and Ar_{12d} is a monovalent group represented by the following formula (50);
Ar_{11d} and Ar_{12d} which is not the monovalent group represented by the following formula (50) is
   a substituted or unsubstituted aryl group including 6 to 50 ring carbon atoms, or
   a substituted or unsubstituted monovalent heterocyclic group including 5 to 50 ring atoms; and
when Ar_{11d} and Ar_{12d} are both the monovalent group represented by the following formula (50), Ar_{11d} and Ar_{12d} which are the monovalent groups represented by the following formula (50) may be the same as or different to each other,

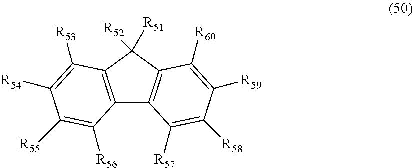

wherein in the formula (50),
R₅₁ and R₅₂ are independently
   a hydrogen atom, a halogen atom, a cyano group, a nitro group,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms, or
   a substituted or unsubstituted cycloalkyl group including 3 to 50 ring carbon atoms;
R₅₁ and R₅₂ do not form a ring by bonding with each other;
one or more sets of adjacent two or more of R₅₃ to R₆₀ form an unsaturated ring represented by the following formula (60) by bonding with each other, or do not form the unsubstituted ring represented by the following formula (60);

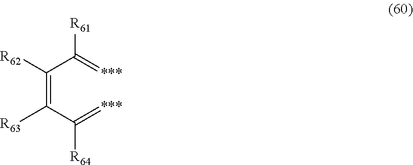

wherein in the formula (60), ***s indicate positions bonding to adjacent two of R₅₃ to R₆₀;
when one or more sets of adjacent two of R₅₃ to R₆₀ form the unsaturated ring represented by the formula (60) by bonding with each other, one of R₅₃ to R₆₀ that do not form the unsaturated ring represented by the formula (60), and R₆₁ to R₆₄ is a single bond bonding with L₁₁ or L₁₂;
when two or more of the unsaturated rings represented by the formula (60) are formed, a plurality of each of R₆₁ to R₆₄ may be the same as or different to each other;
when one or more sets of adjacent two of R₅₃ to R₆₀ do not form the unsaturated ring represented by the formula (60), one of R₅₃ to R₆₀ is a single bond bonding with L₁₁ Or L₁₂;
in the case where the unsaturated ring represented by the formula (60) is formed, and the case where the unsaturated ring represented by the formula (60) is not formed, one or more sets of adjacent two of R₅₃ to R₆₀, that do not form the unsaturated ring represented by the formula (60) and that are not the single bond bonding with L₁₁ or L₁₂, form a substituted or unsubstituted, saturated or unsaturated ring other than the unsaturated ring represented by the formula (60), or do not form a substituted or unsubstituted, saturated or unsaturated ring;
R₅₃ to R₆₀ that do not form the unsaturated ring represented by the formula (60), do not form a substituted or unsubstituted, saturated or unsaturated ring other than the unsaturated ring represented by the formula (60), and are not the single bond bonding with L₁₁ or L₁₂, and R₆₁ to R₆₄ that are not the single bond bonding with L₁₁ or L₁₂ are independently,
   a hydrogen atom, a halogen atom, a cyano group, a nitro group,
   a substituted or unsubstituted alkyl group including 1 to 50 carbon atoms,
   a substituted or unsubstituted alkenyl group including 2 to 50 carbon atoms,
   a substituted or unsubstituted alkynyl group including 2 to 50 carbon atoms,
   a substituted or unsubstituted cycloalkyl group including 3 to 50 ring carbon atoms,
   —Si(R₉₀₁)(R₉₀₂)(R₉₀₃),

### —O—(R₉₀₄),

### —S—(R₉₀₅),

—N(R₉₀₆)(R₉₀₇),
a substituted or unsubstituted aryl group including 6 to 50 ring carbon atoms, or
a substituted or unsubstituted monovalent heterocyclic group including 5 to 50 ring atoms; and
R₉₀₁ to R₉₀₇ are as defined in the formula (11).

In one embodiment, the compound represented by the formula (15) is a compound represented by the following formula (15-1).

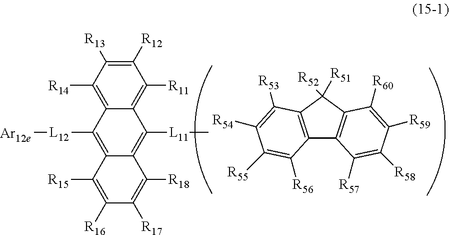

In the formula (15-1), R₁₁ to R₁₈, L₁₁, L₁₂ and R₅₁ to R₆₀ are as defined in the formula (15); and
Ar₁₂ₑ is a substituted or unsubstituted aryl group including 6 to 50 ring carbon atoms, other than the monovalent group represented by the formula (50), or a substituted or unsubstituted monovalent heterocyclic group including 5 to 50 ring atoms.

In one embodiment, R₁₁ to R₁₈ in the formulas (11) to (15) are a hydrogen atom.

In one embodiment, L₁₁ and L₁₂ in the formulas (11) to (15) are independently

a single bond,
an unsubstituted phenylene group,
an unsubstituted naphthylene group,
an unsubstituted biphenyldiyl group, or
an unsubstituted terphenyldiyl group.

In one embodiment, one or both of the first electron-transporting layer and the second electron-transporting layer further includes one or two or more kinds selected from the group consisting of an alkali metal, an alkaline earth metal, a rare earth metal, an alkali metal oxide, an alkali metal halide, an alkaline earth metal oxide, an alkaline earth metal halide, a rare earth metal oxide, a rare earth metal halide, an organic complex containing an alkali metal, an organic complex containing an alkaline earth metal, and an organic complex containing a rare earth metal.

In one embodiment, the second electron-transporting layer further includes one or two or more kinds selected from the group consisting of an alkali metal, an alkaline earth metal, a rare earth metal, an alkali metal oxide, an alkali metal halide, an alkaline earth metal oxide, an alkaline earth metal halide, a rare earth metal oxide, a rare earth metal halide, an organic complex containing an alkali metal, an organic complex containing an alkaline earth metal, and an organic complex containing a rare earth metal.

In one embodiment, a hole-transporting layer is disposed between the anode and the emitting layer.

Hereinafter, a layer configuration of the organic EL device according to an aspect of the invention will be described.

The organic EL device according to an aspect of the invention has an organic layer between a pair of electrodes that are the cathode and the anode. The organic layer contains at least one layer containing an organic compound. Alternatively, the organic layer is formed by stacking a plurality of layers containing an organic compound. The organic layer is formed by stacking a plurality of layers containing an organic compound. The organic layer may have a layer consisting only of one or a plurality of organic compounds. The organic layer may have a layer containing an organic compound and an inorganic compound together. The organic layer may have a layer consisting only of one or a plurality of inorganic compounds.

At least one of the layers contained by the organic layer is an emitting layer. The organic layer may be formed, for example, as one layer of the emitting layer, or may contain other layers which can be adopted in the layer configuration of an organic EL device. Examples of the layers that may be employed in the layer configuration of the organic EL device include, but are not limited to, a hole-transporting region (e.g., a hole-transporting layer, a hole-injecting layer, an electron-blocking layer, an exciton-blocking layer, etc.) disposed between an anode and an emitting layer, an emitting layer, a space layer, and an electron-transporting region (e.g., an electron-transporting layer, an electron-injecting layer, a hole-blocking layer, etc.) disposed between a cathode and an emitting layer.

The organic EL device according to an aspect of the invention may be, for example, a monochromatic emitting device of a fluorescent or phosphorescent type, or a white emitting device of a fluorescent/phosphorescent hybrid type. In addition, it may be a simple type including a single light emitting unit or a tandem type including a plurality of light emitting units.

The “emitting unit” refers to the smallest unit which includes organic layers, in which at least one of the organic layers is an emitting layer, and which emits light by recombination of injected holes and electrons.

The “emitting layer” described in this specification is an organic layer having an emitting function. The emitting layer is, for example, a phosphorescent emitting layer, a fluorescent emitting layer, or the like, and may be a single layer or a plurality of layers.

The light-emitting unit may be of a stacked type including a plurality of a phosphorescent emitting layer and a fluorescent emitting layer, and in this case, for example, it may include a spacing layer between each emitting layer for preventing excitons generated by the phosphorescent emitting layer from diffusing into the fluorescent emitting layer.

The simple type organic EL device includes, for example, a device configuration such as anode/emitting unit/cathode.

Typical layer configurations of the emitting unit are shown below. The layers in parentheses are optional layers.
(a) (hole-injecting layer/) hole-transporting layer/fluorescent emitting layer (/electron-transporting layer/electron-injecting layer)
(b) (hole-injecting layer/) hole-transporting layer/phosphorescent emitting layer (/electron-transporting layer/electron-injecting layer)
(c) (hole-injecting layer/) hole-transporting layer/first fluorescent emitting layer/second fluorescent emitting layer (/electron-transporting layer/electron-injecting layer)
(d) (hole-injecting layer/) hole-transporting layer/first phosphorescent emitting layer/second phosphorescent emitting layer (/electron-transporting layer/electron-injecting layer)
(e) (hole-injecting layer/) hole-transporting layer/phosphorescent emitting layer/spacing layer/fluorescent emitting layer (/electron-transporting layer/electron-injecting layer)
(f) (hole-injecting layer/) hole-transporting layer/first phosphorescent emitting layer/second phosphorescent emitting layer/spacing layer/fluorescent emitting layer (/electron-transporting layer/electron-injecting layer)
(g) (hole-injecting layer/) hole-transporting layer/first phosphorescent layer/spacing layer/second phosphorescent emitting layer/spacing layer/fluorescent emitting layer (/electron-transporting layer/electron-injecting layer)
(h) (hole-injecting layer/) hole-transporting layer/phosphorescent emitting layer/spacing layer/first fluorescent emitting layer/second fluorescent emitting layer (/electron-transporting layer/electron-injecting layer)
(i) (hole-injecting layer/) hole-transporting layer/electron-blocking layer/fluorescent emitting layer (/electron-transporting layer/electron-injecting layer)
(j) (hole-injecting layer/) hole-transporting layer/electron-blocking layer/phosphorescent emitting layer (/electron-transporting layer/electron-injecting layer)
(k) (hole-injecting layer/) hole-transporting layer/exciton-blocking layer/fluorescent emitting layer (/electron-transporting layer/electron-injecting layer)
(l) (hole-injecting layer/) hole-transporting layer/exciton-blocking layer/phosphorescent emitting layer (/electron-transporting layer/electron-injecting layer)
(m) (hole-injecting layer/) first hole-transporting layer/second hole-transporting layer/fluorescent emitting layer (/electron-transporting layer/electron-injecting layer)
(n) (hole-injecting layer/) first hole-transporting layer/second hole-transporting layer/fluorescent emitting layer (/first electron-transporting layer/second electron-transporting layer/electron-injecting layer)
(o) (hole-injecting layer/) first hole-transporting layer/second hole-transporting layer/phosphorescent emitting layer (/electron-transporting layer/electron-injecting layer)
(p) (hole-injecting layer/) first hole-transporting layer/second hole-transporting layer/phosphorescent emitting layer (/first electron-transporting layer/second electron-transporting layer/electron-injecting layer)
(q) (hole-injecting layer/) hole-transporting layer/fluorescent emitting layer/hole-blocking layer (/electron-transporting layer/electron-injecting layer)
(r) (hole-injecting layer/) hole-transporting layer/phosphorescent emitting layer/hole-blocking layer (/electron-transporting layer/electron-injecting layer)
(s) (hole-injecting layer/) hole-transporting layer/fluorescent emitting layer/exciton-blocking layer (/electron-transporting layer/electron-injecting layer)
(t) (hole-injecting layer/) hole-transporting layer/phosphorescent emitting layer/exciton-blocking layer (/electron-transporting layer/electron-injecting layer)

However, the layer configuration of the organic EL device according to one aspect of the invention is not limited thereto. For example, when the organic EL device has a hole-injecting layer and a hole-transporting layer, it is preferred that a hole-injecting layer be provided between the hole-transporting layer and the anode. Further, when the organic EL device has an electron-injecting layer and an electron-transporting layer, it is preferred that an electron-injecting layer be provided between the electron-transporting layer and the cathode. Further, each of the hole-injecting layer, the hole-transporting layer, the electron-transporting layer and the electron-injecting layer may be constituted of a single layer or of a plurality of layers.

The plurality of phosphorescent emitting layers, and the plurality of the phosphorescent emitting layer and the fluorescent emitting layer may be emitting layers that emit mutually different colors. For example, the emitting unit (f) may contain a hole-transporting layer/first phosphorescent layer (red light emission)/second phosphorescent emitting layer (green light emission)/spacing layer/fluorescent emitting layer (blue light emission)/electron-transporting layer.

An electron-blocking layer may be provided between each light emitting layer and the hole-transporting layer or the spacing layer. Further, a hole-blocking layer may be provided between each emitting layer and the electron-transporting layer. By providing the electron-blocking layer or the hole-blocking layer, it is possible to confine electrons or holes in the emitting layer, thereby to improve the recombination probability of carriers in the emitting layer, and to improve luminous efficiency.

As a representative device configuration of a tandem type organic EL device, for example, a device configuration such as anode/first emitting unit/intermediate layer/second emitting unit/cathode can be given.

The first emitting unit and the second emitting unit are independently selected from the above-mentioned emitting units, for example.

The intermediate layer is also generally referred to as an intermediate electrode, an intermediate conductive layer, a charge generating layer, an electron withdrawing layer, a connecting layer, a connector layer, or an intermediate insulating layer. The intermediate layer is a layer that supplies electrons to the first emitting unit and holes to the second emitting unit, and can be formed of known materials.

Hereinbelow, an explanation will be made on function, materials, etc. of each layer constituting the organic EL device described in this specification.

### (Substrate)

The substrate is used as a support of the organic EL device. The substrate preferably has a light transmittance of 50% or more in the visible light region within a wavelength of 400 to 700 nm, and a smooth substrate is preferable. Examples of the material of the substrate include soda-lime glass, aluminosilicate glass, quartz glass, plastic and the like. As the substrate, a flexible substrate can be used. The flexible substrate means a substrate that can be bent (flexible), and examples thereof include a plastic substrate and the like. Specific examples of the material for forming the plastic substrate include polycarbonate, polyallylate, polyether sulfone, polypropylene, polyester, polyvinyl fluoride, polyvinyl chloride, polyimide, polyethylene naphthalate and the like. Also, an inorganic vapor deposited film can be used.

### (Anode)

As the anode, for example, it is preferable to use a metal, an alloy, a conductive compound, a mixture thereof or the like, which has a high work function (specifically, 4.0 eV or more). Specific examples of the material of the anode include indium oxide-tin oxide (ITO: Indium Tin Oxide), indium oxide-tin oxide containing silicon or silicon oxide, indium oxide-zinc oxide, indium oxide containing tungsten oxide or zinc oxide, graphene and the like. In addition, it is possible to use gold, silver, platinum, nickel, tungsten, chromium, molybdenum, iron, cobalt, copper, palladium, titanium, nitrides of these metals (e.g. titanium nitride) and the like.

The anode is normally formed by depositing these materials on the substrate by a sputtering method. For example, indium oxide-zinc oxide can be formed by a sputtering method by using a target in which 1 to 10 mass % zinc oxide is added to indium oxide. Further, indium oxide containing tungsten oxide or zinc oxide can be formed by a sputtering method by using a target in which 0.5 to 5 mass % of tungsten oxide or 0.1 to 1 mass % of zinc oxide is added to indium oxide.

As the other methods for forming the anode, a vacuum deposition method, a coating method, an inkjet method, a spin coating method or the like can be given. When silver paste or the like is used, it is possible to use a coating method, an inkjet method or the like.

The hole-injecting layer formed in contact with the anode is formed by using a material that allows easy hole injection regardless of the work function of the anode. For this reason, in the anode, it is possible to use a common electrode material, for example, a metal, an alloy, a conductive compound and a mixture thereof. Specifically, materials having a small work function such as alkaline metals such as lithium and cesium; magnesium; alkaline earth metals such as calcium and strontium; alloys containing these metals (for example, magnesium-silver and aluminum-lithium); rare earth metals such as europium and ytterbium; and an alloy containing rare earth metals can also be used for the anode.

### (Hole-Injecting Layer)

A hole-injecting layer is a layer that contains a substance having a high hole-injecting property and has a function of injecting holes from the anode to the organic layer. As the substance having a high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, an electron-attracting (acceptor) compound, a polymeric compound (oligomer, dendrimer, polymer, etc.) and the like can be given. Among these, an aromatic amine compound and an acceptor compound are preferable, with an acceptor compound being more preferable.

Specific examples of the aromatic amine compound include 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), and the like.

The acceptor compound is preferably, for example, a heterocyclic ring derivative having an electron-attracting group, a quinone derivative having an electron-attracting group, an arylborane derivative, a heteroarylborane derivative, and the like, and specific examples include hexacyanohexaazatriphenylene, 2,3,5,6-tetrafluoro-7,7,8,8-tetracyanoquinodimethane (abbreviation: F4TCNQ), 1,2,3-tris[(cyano)(4-cyano-2,3,5,6-tetrafluorophenyl)methylene]cyclopropane, and the like.

When the acceptor compound is used, it is preferred that the hole-injecting layer further comprise a matrix material. As the matrix material, a material known as the material for an organic EL device can be used. For example, an electron-donating (donor) compound is preferable.

### (Hole-Transporting Layer)

The hole-transporting layer is a layer that comprises a high hole-transporting property, and has a function of transporting holes from the anode to the organic layer.

As the substance having a high hole-transporting property, a substance having a hole mobility of 10⁻⁶ cm²/(V·s) or more is preferable. For example, an aromatic amine compound, a carbazole derivative, an anthracene derivative, a polymeric compound, and the like can be given.

Specific examples of the aromatic amine compound include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluoren-2-yl)-N-phenylamino]biphenyl (abbreviation: DFLDPBi), 4,4′,4″-tris(N, N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(spiro-9,9′-bifluoren-2-yl)-N-phenylamino]biphenyl (abbreviation: BSPB), and the like.

Specific examples of the carbazole derivative include 4,4′-di(9-carbazolyl)biphenyl (abbreviation: CBP), 9-[4-(9-carbazolyl)phenyl]-10-phenylanthracene (abbreviation: CzPA), 9-phenyl-3-[4-(10-phenyl-9-anthryl)phenyl]-9H-carbazole (abbreviation: PCzPA) and the like.

Specific examples of the anthracene derivative include 2-t-butyl-9,10-di(2-naphthyl)anthracene (t-BuDNA), 9,10-di(2-naphthyl)anthracene (DNA), 9,10-diphenylanthracene (DPAnth), and the like.

Specific examples of the polymeric compound include poly(N-vinylcarbazole) (abbreviation: PVK), poly(4-vinyltriphenylamine) (abbreviation: PVTPA) and the like.

As long as a compound other than those mentioned above, that has a higher hole-transporting property as compared with electron-transporting property, such a compound can be used for the hole-transporting layer.

The hole-transporting layer may be a single layer or may be a stacked layer of two or more layers. In this case, it is preferred to arrange a layer that contains a substance having a larger energy gap among substances having a higher hole-transporting property, on a side nearer to the emitting layer.

### (Emitting Layer)

The emitting layer is a layer containing a substance having a high emitting property (dopant material). As the dopant material, various types of material can be used. For example, a fluorescent emitting compound (fluorescent dopant), a phosphorescent emitting compound (phosphorescent dopant) or the like can be used. A fluorescent emitting compound is a compound capable of emitting light from the singlet excited state, and an emitting layer containing a fluorescent emitting compound is called as a fluorescent emitting layer. Further, a phosphorescent emitting compound is a compound capable of emitting light from the triplet excited state, and an emitting layer containing a phosphorescent emitting compound is called as a phosphorescent emitting layer.

The emitting layer normally contains a dopant material and a host material that allows the dopant material to emit light efficiently. In some literatures, a dopant material may be called as a guest material, an emitter, or an emitting material. In some literatures, a host material is called as a matrix material.

A single emitting layer may include a plurality of dopant materials and a plurality of host materials. Further, a plurality of emitting layers may be present.

In this specification, a host material combined with the fluorescent dopant is referred to as a “fluorescent host” and a host material combined with the phosphorescent dopant is referred to as the “phosphorescent host”. Note that the fluorescent host and the phosphorescent host are not classified only by the molecular structure. The phosphorescent host is a material for forming a phosphorescent emitting layer containing a phosphorescent dopant, but it does not mean that it cannot be used as a material for forming a fluorescent emitting layer. The same can be applied to the fluorescent host.

The content of the dopant material in the emitting layer is not particularly limited, but from the viewpoint of adequate luminescence and concentration quenching, it is preferable, for example, to be 0.1 to 70 mass %, more preferably 0.1 to 30 mass %, more preferably 1 to 30 mass %, still more preferably 1 to 20 mass %, and particularly preferably 1 to 10 mass %.

### <Fluorescent Dopant>

As the fluorescent dopant, a fused polycyclic aromatic derivative, a styrylamine derivative, a fused ring amine derivative, a boron-containing compound, a pyrrole derivative, an indole derivative, a carbazole derivative can be given, for example. Among these, a fused ring amine derivative, a boron-containing compound, and a carbazole derivative are preferable.

As the fused ring amine derivative, a diaminopyrene derivative, a diaminochrysene derivative, a diaminoanthracene derivative, a diaminofluorene derivative, a diaminofluorene derivative with which one or more benzofuro skeletons are fused, and the like can be given.

As the boron-containing compound, a pyrromethene derivative, a triphenylborane derivative and the like can be given.

Examples of the blue fluorescent dopant include a pyrene derivative, a styrylamine derivative, a chrysene derivative, a fluoranthene derivative, a fluorene derivative, a diamine derivative, a triarylamine derivative, and the like. Specifically, N, N′-bis[4-(9H-carbazol-9-yl)phenyl]-N, N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(10-phenyl-9-anthryl)-4′-(9-phenyl-9H-carbazol-3-yl)triphenylamine (abbreviation: PCBAPA) and the like can be given.

As the green fluorescent dopant, an aromatic amine derivative and the like can be given, for example. Specifically, N-(9,10-diphenyl-2-anthryl)-N, 9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]—N-[4-(9H-carbazol-9-yl) phenyl]-N-phenylanthracene-2-amine (abbreviation: 2YGABPhA), N, N, 9-triphenylanthracene-9-amine (abbreviation: DPhAPhA), and the like can be given.

As the red fluorescent dopant, a tetracene derivative, a diamine derivative or the like can be given. Specifically, N,N,N′,N′-tetrakis(4-methylphenyl)tetracen-5,11-diamine (abbreviation: p-mPhTD), 7,14-diphenyl-N,N,N′,N′-tetrakis(4-methylphenyl)acenaphtho[1,2-a]fluoranthen-3,10-diamine (abbreviation: p-mPhAFD) and the like can be given.

### <Phosphorescent Dopant>

As the phosphorescent dopant, a phosphorescent light-emitting heavy metal complex and a phosphorescent light-emitting rare earth metal complex can be given.

As the heavy metal complex, an iridium complex, an osmium complex, a platinum complex and the like can be given. As the heavy metal complex, an ortho-metalated complex of a metal selected from iridium, osmium and platinum.

As the rare earth metal complexes include a terbium complex, a europium complex and the like. Specifically, tris(acetylacetonate)(monophenanthroline)terbium (III) (abbreviation: Tb(acac)₃(Phen)), tris(1,3-diphenyl-1,3-propandionate)(monophenanthroline)europium (III) (abbreviation: Eu(DBM)₃(Phen)), tris[1-(2-thenoyl)-3,3,3-trifluoroacetonate](monophenanthroline)europium (III) (abbreviation: Eu(TTA)₃(Phen)) and the like can be given. These rare earth metal complexes are preferable as phosphorescent dopants since rare earth metal ions emit light due to electronic transition between different multiplicity.

As the blue phosphorescent dopant, an iridium complex, an osmium complex, a platinum complex, or the like can be given, for example. Specific examples include bis[2-(4′,6′-difluorophenyl)pyridinato-N, C2′]iridium (III) tetrakis(1-pyrazolyl)borate (abbreviation: Flr6), bis[2-(4′,6′-difluorophenyl)pyridinato-N, C2′]iridium (III) picolinate (abbreviation: Flrpic), bis[2-(3′,5′-bistrofluoromethylphenyl)pyridinato-N, C2′]iridium (III) picolinate (abbreviation: Ir(CF₃ppy)₂(pic)), bis[2-(4′,6′-difluorophenyl)pyridinato-N, C2′]iridium (III) acetylacetonate (abbreviation: Flracac), and the like.

As the green phosphorescent dopant, an iridium complex or the like can be given, for example. Specific examples include tris(2-phenylpyridinato-N, C2′)iridium (III) (abbreviation: Ir(ppy)₃), bis(2-phenylpyridinato-N,C2′)iridium (III) acetylacetonate (abbreviation: Ir(ppy)₂(acac)), bis(1,2-diphenyl-1H benzimidazolate)iridium (III) acetylacetonate (abbreviation: Ir(pbi)₂(acac)), bis(benzo[h]quinolinato)iridium (III) acetylacetonate (abbreviation: Ir(bzq)₂(acac)), and the like.

As the red phosphorescent dopant, an iridium complex, a platinum complex, a terbium complex, a europium complex and the like can be given. Specifically, bis[2-(2′-benzo[4,5-a] thienyl)pyridinato-N, C3′]iridium (III) acetylacetonate (abbreviation: Ir(btp)₂(acac)), bis(1-phenylisoquinolinato-N,C2′)iridium (III) acetylacetonate (abbreviation: Ir(piq)₂(acac)), (acetylacetonate)bis[2,3-bis(4-fluorophenyl)quinoxalinato]iridium (III) (abbreviation: Ir(Fdpq)₂(acac)), 2,3,7,8,12,13,17,18-octaethyl-21H,23H-porphyrin platinum (II) (abbreviation: PtOEP), and the like.

### <Host Material>

Examples of the host material include metal complexes such as an aluminum complex, a beryllium complex, and a zinc complex; heterocyclic compounds such as an indole derivative, a pyridine derivative, a pyrimidine derivative, a triazine derivative, a quinoline derivative, an isoquinoline derivative, a quinazoline derivative, a dibenzofuran derivative, a dibenzothiophene derivative, an oxadiazole derivative, a benzimidazole derivative, a phenanthroline derivative; fused aromatic compounds such as a naphthalene derivative, a triphenylene derivative, a carbazole derivative, an anthracene derivative, a phenanthrene derivative, a pyrene derivative, a chrysene derivative, a naphthacene derivative, and a fluoranthene derivative; and aromatic amine compounds such as a triarylamine derivative, and a fused polycyclic aromatic amine derivative, and the like. Plural types of host materials can be used in combination.

Specific examples of the metal complex include tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), bis[2-(2-benzothiazolyl) phenolato]zinc(II) (abbreviation: ZnBTZ), and the like.

Specific examples of the heterocyclic compound include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), bathocuproine (abbreviation: BCP), and the like.

Specific examples of the fused aromatic compound include 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), 6,12-dimethoxy-5,11-diphenylchrysene, and the like.

Specific examples of the aromatic amine compound include N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazol-3-amine (abbreviation: PCAPA), N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 4,4′-bis[N-(1-naphthyl)-N-phenylamino]biphenyl (abbreviation: NPB or α-NPD), N,N′-bis(3-methylphenyl)-N, N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), 4,4′-bis[N-(spiro-9,9′-bifluoren-2-yl)-N-phenylamino]biphenyl (abbreviation: BSPB), and the like.

As the fluorescent host material, a compound having a higher singlet energy level as compared with a fluorescent dopant is preferable. For example, a heterocyclic compound, a fused aromatic compound, and the like can be given. As fused aromatic compounds, for example, anthracene derivatives, pyrene derivatives, chrysene derivatives, and naphthacene derivatives are preferred.

As the phosphorescent host, a compound having a higher triplet energy level as compared with a phosphorescent dopant is preferable. For example, a metal complex, a heterocyclic compound, a fused aromatic compound and the like can be given. Among these, an indole derivative, a carbazole derivative, a pyridine derivative, a pyrimidine derivative, a triazine derivative, a quinoline derivative, an isoquinoline derivative, a quinazoline derivative, a dibenzofuran derivative, a dibenzothiophene derivative, a naphthalene derivative, a triphenylene derivative, a phenanthrene derivative, a fluoranthene derivative and the like are preferable.

### (Electron-Transporting Layer)

An electron-transporting layer is a layer that comprises a substance having a high electron-transporting property. As the substance having a high electron-transporting property, a substance having an electron mobility of 10⁻⁶ cm²/Vs or more is preferable. For example, the compound represented by the above formula (A1), a metal complex, an aromatic heterocyclic compound, an aromatic hydrocarbon compound, a polymeric compound and the like can be given.

As the metal complex, an aluminum complex, a beryllium complex, a zinc complex and the like can be given. Specific examples of the metal complex include tris (8-quinolinolato) aluminum (III) (abbreviation: Alq), tris (4-methyl-8-quinolinolato) aluminum (abbreviation: Almq3), bis (10-hydroxybenzo[h]quinolinato) beryllium (abbreviation: BeBq2), bis (2-methyl-8-quinolinolato) (4-phenylphenolato) aluminum (III) (abbreviation: BAlq), bis (8-quinolinolato) zinc (II) (abbreviation: Znq), bis [2-(2-benzoxazolyl) phenolato]zinc (II) (abbreviation: ZnPBO), bis [2-(2-benzothiazolyl) phenolato] zinc(II) (abbreviation: ZnBTZ), and the like.

As the aromatic heterocyclic compound, imidazole derivatives such as a benzimidazole derivative, an imidazopyridine derivative and a benzimidazophenanthridine derivative; azine derivatives such as a pyrimidine derivative and a triazine derivative; compounds having a nitrogen-containing 6-membered ring structure such as a quinoline derivative, an isoquinoline derivative, and a phenanthroline derivative (also including one having a phosphine oxide-based substituent on the heterocyclic ring) and the like can be given. Specifically, 2-(4-biphenylyl)-5-(4-tert-butylphenyl)-1,3,4-oxadiazole (abbreviation: PBD), 1,3-bis [5-(p-tert-butylphenyl)-1,3,4-oxadiazol-2-yl] benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), 4,4′-bis (5-methylbenzoxazol-2-yl) stilbene (abbreviation: BzOs), and the like can be given.

As the aromatic hydrocarbon compound, an anthracene derivative, a fluoranthene derivative and the like can be given, for example.

As specific examples of the polymeric compound, poly [(9,9-dihexylfluoren-2,7-diyl)-co-(pyridine-3,5-diyl)] (abbreviation: PF-Py), poly [(9,9-dioctylfluoren-2,7-diyl)-co-(2,2′-bipyridin-6,6′-diyl)] (abbreviation: PF-BPy) and the like can be given.

As long as a compound other than those mentioned above, that has a higher electron-transporting property as compared with hole-transporting property, such a compound may be used in the electron-transporting layer.

The electron-transporting layer may be a single layer, or a stacked layer of two or more layers. In this case, it is preferable to arrange a layer that contains a substance having a larger energy gap, among substances having a high electron-transporting property, on the side nearer to the emitting layer.

The electron-transporting layer may contain a metal such as an alkali metal, magnesium, an alkaline earth metal, or an alloy containing two or more of these metals; a metal compound such as an alkali metal compound such as 8-quinolinolato lithium (Liq), or an alkaline earth metal compound. When a metal such as an alkali metal, magnesium, an alkaline earth metal, or an alloy containing two or more of these metals is contained in the electron-transporting layer, the content of the metal is not particularly limited, but is preferably from 0.1 to 50 mass %, more preferably from 0.1 to 20 mass %, further preferably from 1 to 10 mass %.

When a metal compound such as an alkali metal compound or an alkaline earth metal compound is contained in the electron-transporting layer, the content of the metal compound is preferably from 1 to 99 mass %, more preferably from 10 to 90 mass %. When plural electron-transporting layers are provided, the layer on the emitting layer side can be formed only from the metal compound as mentioned above.

### (Electron-Injecting Layer)

The electron-injecting layer is a layer that contains a substance having a high electron-injecting property, and has the function of efficiently injecting electrons from a cathode to an emitting layer. Examples of the substance that has a high electron-injecting property include an alkali metal, magnesium, an alkaline earth metal, a compound thereof, and the like. Specific examples thereof include lithium, cesium, calcium, lithium fluoride, cesium fluoride, calcium fluoride, lithium oxide, and the like. In addition, a material in which an alkali metal, magnesium, an alkaline earth metal, or a compound thereof is incorporated to an electron-transporting substance having an electron-transporting property, for example, Alq incorporated with magnesium, may also be used.

Alternatively, a composite material that includes an organic compound and a donor compound may also be used in the electron-injecting layer. Such a composite material is excellent in the electron-injecting property and the electron-transporting property since the organic compound receives electrons from the donor compound.

The organic compound is preferably a substance excellent in transporting property of the received electrons, and specifically, for example, the metal complex, the aromatic heterocyclic compound, and the like, which are a substance that has a high electron-transporting property as mentioned above, can be used.

Any material capable of donating electrons to an organic compound can be used as the donor compound. Examples thereof include an alkali metal, magnesium, an alkaline earth metal, a rare earth metal and the like. Specific examples thereof include lithium, cesium, magnesium, calcium, erbium, ytterbium, and the like. Further, an alkali metal oxide and an alkaline earth metal oxide are preferred, and examples thereof include lithium oxide, calcium oxide, barium oxide, and the like. Lewis bases such as magnesium oxide can also be used. Alternatively, an organic compound such as tetrathiafulvalene (abbreviation: TTF) can be used.

### (Cathode)

For the cathode, a metal, an alloy, an electrically conductive compound, and a mixture thereof, each having a small work function (specifically, a work function of 3.8 eV or less) are preferably used. Specific examples of the material for the cathode include alkali metals such as lithium and cesium; magnesium; alkaline earth metals such as calcium, and strontium; alloys containing these metals (for example, magnesium-silver, and aluminum-lithium); rare earth metals such as europium and ytterbium; alloys containing a rare earth metal, and the like.

The cathode is usually formed by a vacuum vapor deposition or a sputtering method. Further, in the case of using a silver paste or the like, a coating method, an inkjet method, or the like can be employed.

In the case where the electron-injecting layer is provided, a cathode can be formed from a substance selected from various electrically conductive materials such as aluminum, silver, ITO, graphene, indium oxide-tin oxide containing silicon or silicon oxide, regardless of the work function value. These electrically conductive materials are made into films by using a sputtering method, an inkjet method, a spin coating method, or the like.

### (Insulating Layer)

In the organic EL device, pixel defects based on leakage or a short circuit are easily generated since an electric field is applied to a thin film. In order to prevent this, an insulating thin layer may be inserted between a pair of electrodes.

Examples of substances used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, vanadium oxide, and the like. A mixture thereof may be used in the insulating layer, and a stacked body of a plurality of layers that include these substances can be also used for the insulating layer.

### (Spacing Layer)

The spacing layer is a layer provided between a fluorescent emitting layer and a phosphorescent emitting layer when the fluorescent emitting layer and the phosphorescent emitting layer are stacked, in order to prevent diffusion of excitons generated in the phosphorescent emitting layer to the fluorescent emitting layer or in order to adjust the carrier balance. Further, the spacing layer can be provided between plural phosphorescent emitting layers.

Since the spacing layer is provided between the emitting layers, the material used for the spacing layer is preferably a substance that has both electron-transporting property and hole-transporting property. In order to prevent diffusion of the triplet energy in adjacent phosphorescent emitting layers, it is preferred that the material used for the spacing layer have a triplet energy of 2.6 eV or more.

As the material used for the spacing layer, the same materials as those used in the above-mentioned hole-transporting layer can be given.

### (Electron-Blocking Layer, Hole-Blocking Layer, Exciton-Blocking Layer)

An electron-blocking layer, a hole-blocking layer, an exciton (triplet)-blocking layer, and the like may be provided in adjacent to the emitting layer.

The electron-blocking layer has a function of preventing leakage of electrons from the emitting layer to the hole-transporting layer. The hole-blocking layer has a function of preventing leakage of holes from the emitting layer to the electron-transporting layer. The exciton-blocking layer has a function of preventing diffusion of excitons generated in the emitting layer to the adjacent layers to confine the excitons within the emitting layer.

### (Capping Layer)

The organic EL device can be provided with a capping layer above the cathode in order to adjust the intensity of the outcoupled light with the optical interference effect.

For the capping layer, for example, a polymer compound, a metal oxide, a metal fluoride, a metal boride, silicon nitride, a silicon compound (silicon oxide, etc.) and the like can be used.

Further, an aromatic amine derivative, an anthracene derivative, a pyrene derivative, a fluorene derivative, and a dibenzofuran derivative can also be used for the capping layer.

A stacked body in which layers containing these substances are stacked can also be used as a capping layer.

### (Intermediate Layer)

In tandem-type organic EL device, an intermediate layer is provided.

### (Method for Forming a Layer)

The method for forming each layer of the organic EL device is not particularly limited unless otherwise specified. As the film forming method, a known film-forming method such as a dry film-forming method, a wet film-forming method or the like can be used. Specific examples of the dry film-forming method include a vacuum deposition method, a sputtering method, a plasma method, an ion plating method, and the like. Specific examples of the wet film-forming method include various coating methods such as a spin coating method, a dipping method, a flow coating method, and an inkjet method.

### (Film Thickness)

The film thickness of each layer of the organic EL device is not particularly limited unless otherwise specified. If the film thickness is too small, defects such as pinholes are likely to occur to make it difficult to obtain an enough luminance. On the other hand, if the film thickness is too large, a high driving voltage is required to be applied, leading to a lowering in efficiency. In this respect, the film thickness is preferably 1 nm to 10 μm, and more preferably 1 nm to 0.2 μm.

### [Electronic Apparatus]

The electronic apparatus according to one aspect of the invention includes the above-described organic EL device according to one aspect of the invention. Examples of the electronic apparatus include display parts such as an organic EL panel module; display devices of television sets, mobile phones, smart phones, personal computers, and the like; and emitting devices of a lighting device and a vehicle lighting device.

### EXAMPLES

Next, the invention will be described in more detail by referring to Examples and Comparative Examples, but the invention is not limited in any way to the description of these Examples.

### <Compound>

The compounds represented by the formula (1) or formula (A1) used for fabricating the organic EL device of Examples 1 to 13 are shown below.

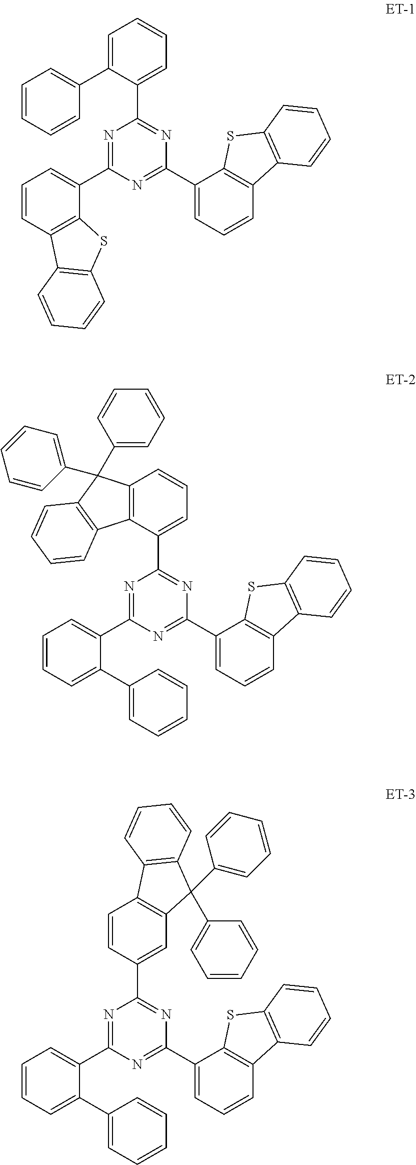 [SMILES: c1ccc(-c2ccccc2-c2nc(-c3cccc4c3sc3ccccc34)nc(-c3cccc4c3sc3ccccc34)n2)cc1 ; c1ccc(-c2ccccc2-c2nc(-c3cccc4c3-c3ccccc3C4(c3ccccc3)c3ccccc3)nc(-c3cccc4c3sc3ccccc34)n2)cc1 ; c1ccc(-c2ccccc2-c2nc(-c3ccc4c(c3)C(c3ccccc3)(c3ccccc3)c3ccccc3-4)nc(-c3cccc4c3sc3ccccc34)n2)cc1]

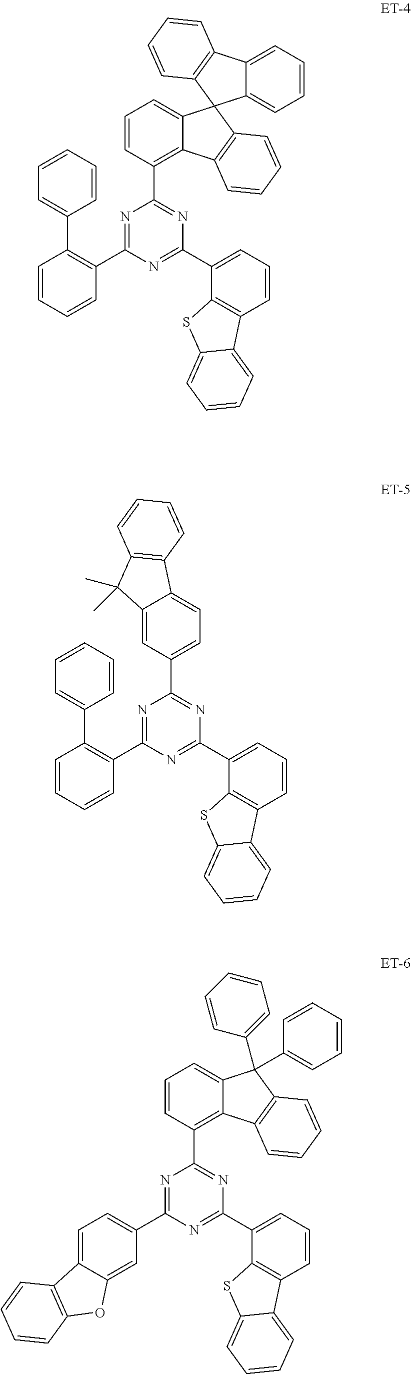 [SMILES: c1ccc(-c2ccccc2-c2nc(-c3cccc4c3-c3ccccc3C43c4ccccc4-c4ccccc43)nc(-c3cccc4c3sc3ccccc34)n2)cc1 ; CC1(C)c2ccccc2-c2ccc(-c3nc(-c4ccccc4-c4ccccc4)nc(-c4cccc5c4sc4ccccc45)n3)cc21 ; c1ccc(C2(c3ccccc3)c3ccccc3-c3c(-c4nc(-c5ccc6c(c5)oc5ccccc56)nc(-c5cccc6c5sc5ccccc56)n4)cccc32)cc1]

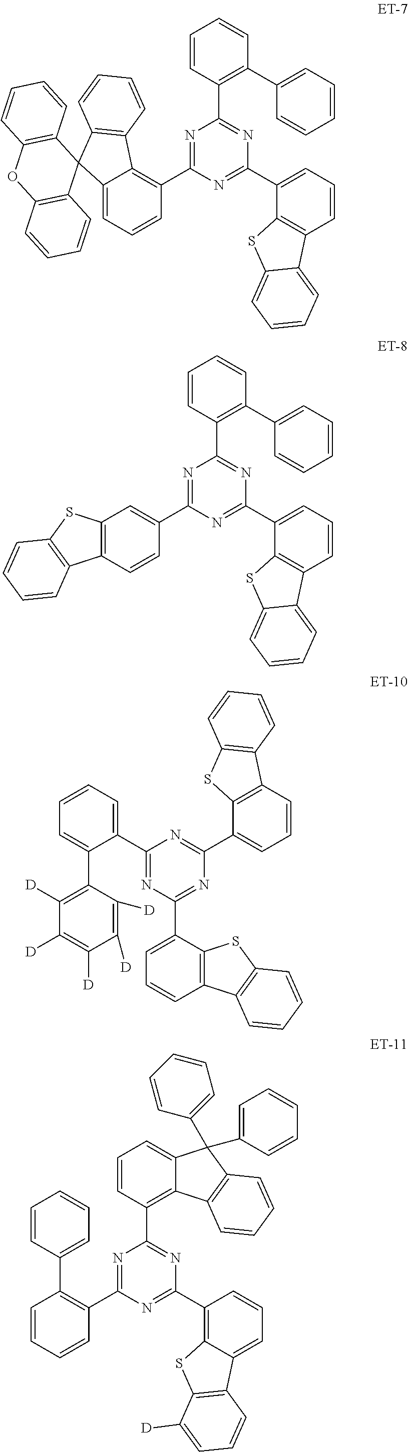

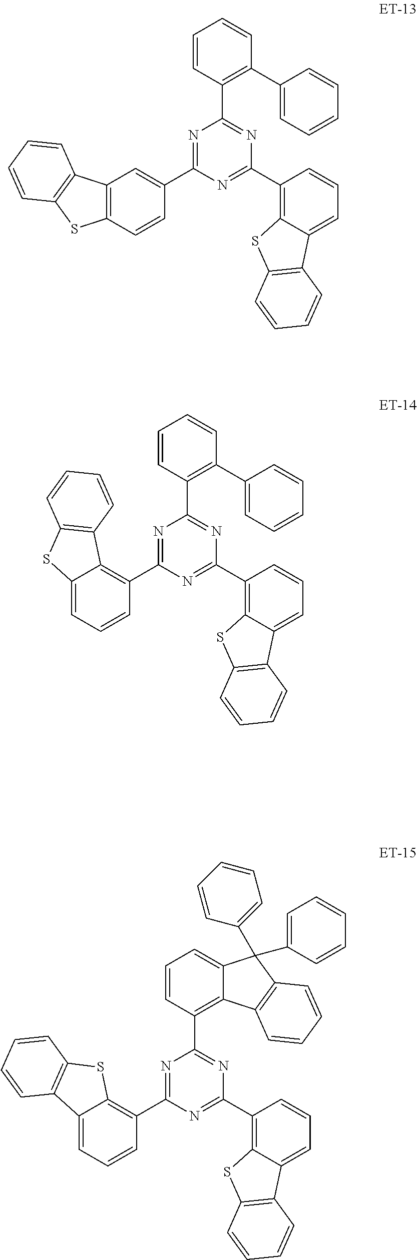 [SMILES: c1ccc(-c2ccccc2-c2nc(-c3ccc4sc5ccccc5c4c3)nc(-c3cccc4c3sc3ccccc34)n2)cc1 ; c1ccc(-c2ccccc2-c2nc(-c3cccc4c3sc3ccccc34)nc(-c3cccc4sc5ccccc5c34)n2)cc1 ; c1ccc(C2(c3ccccc3)c3ccccc3-c3c(-c4nc(-c5cccc6c5sc5ccccc56)nc(-c5cccc6c5sc5ccccc56)n4)cccc32)cc1]

The compounds used for fabricating the organic EL device of Comparative Example 1 are shown below.

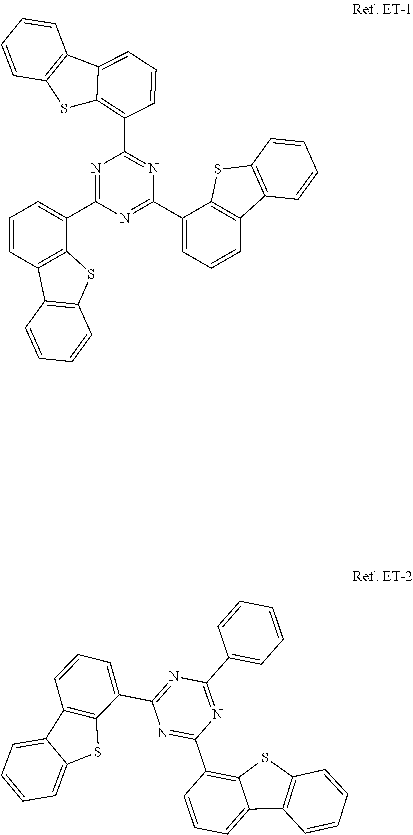 [SMILES: c1ccc2c(c1)sc1c(-c3nc(-c4cccc5c4sc4ccccc45)nc(-c4cccc5c4sc4ccccc45)n3)cccc12 ; c1ccc(-c2nc(-c3cccc4c3sc3ccccc34)nc(-c3cccc4c3sc3ccccc34)n2)cc1]

The other compounds used for fabricating the organic EL device of Examples 1 to 3 and Comparative Example 1 are shown below.

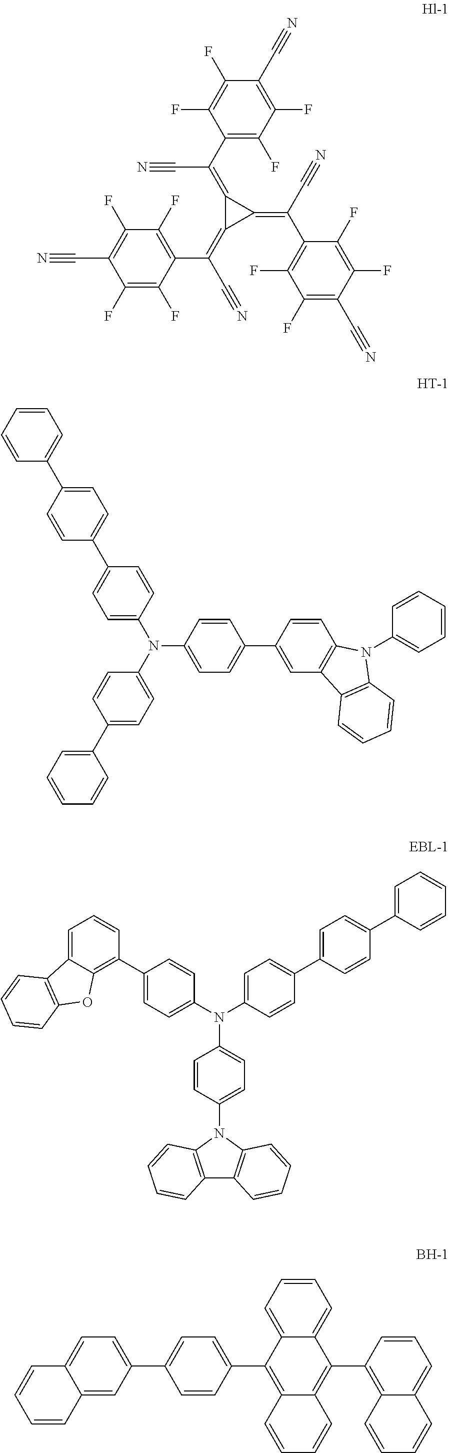

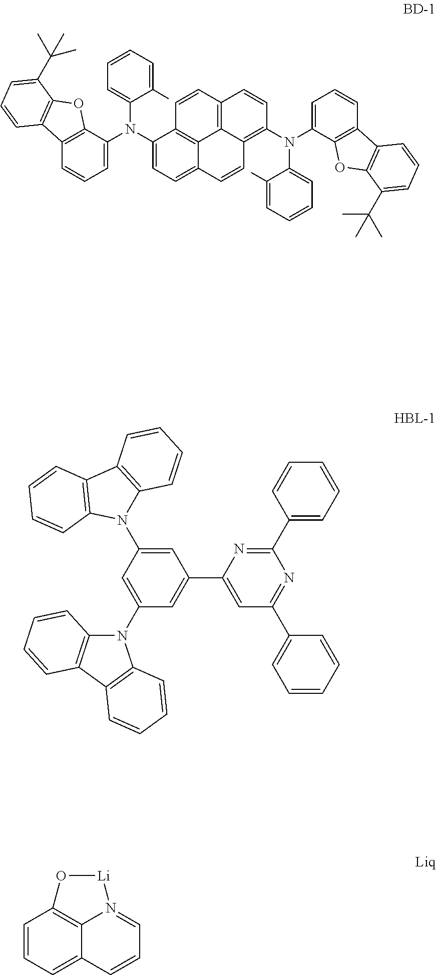

### <Calculation of Affinity Values for Compounds>

For the chemical structure formula of the compounds shown in Table 1 below, used in the following Examples and Comparative Examples, the quantum chemical calculation program (Gaussian 09, Revision E (Gaussian Inc.); calculation method: B3LYP/6-31G* (meaning that B3LYP was used for theory, and 6-31G* was used for the basis function)) was used to calculate electrons affinity (affinity value: Af). The results are shown in Table 1 and Table 2 below.

TABLE 1

| Compound | Af (eV) |
|---|---|
| ET-1 | 1.93 |
| ET-2 | 1.86 |
| ET-3 | 1.91 |
| ET-4 | 1.87 |
| ET-5 | 1.90 |
| ET-7 | 1.93 |
| ET-10 | 1.93 |
| ET-11 | 1.86 |
| ET-13 | 1.90 |
| ET-14 | 1.93 |
| Ref. ET-1 | 2.00 |
| Ref. ET-2 | 1.98 |

### <Fabrication of Organic EL Device>

The organic EL device was fabricated and evaluated as follows.

### Example 1

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HI-1 and a compound HT-1 were co-deposited to be 3 mass % in a proportion of the compound HT-1 on a surface on the side on which the transparent electrode was formed so as to cover the transparent electrode to form a hole-injecting layer having a thickness of 10 nm.

Next, a compound HT-1 was deposited on the hole-injecting layer to form a first hole-transporting layer having a thickness of 80 nm on the HI-1:HT-1 film.

Next, a compound EBL-1 was deposited on this first hole-transporting layer to form a second hole-transporting layer (electron-blocking layer) having a thickness of 5 nm.

Next, a compound BH-1 (host material) and a compound BD-1 (dopant material) were co-evaporated on the second hole-transporting layer to be 4 mass % in a proportion of the compound BD-1 to form an emitting layer having a thickness of 25 nm.

Next, a compound HBL-1 was deposited on the emitting layer to form a first electron-transporting layer (hole-blocking layer) having a thickness of 5 nm.

Next, a compound ET-1 and Liq were co-evaporated on this first electron-transporting layer to be 50 mass % in a proportion of Liq to form a second electron-transporting layer having a thickness of 20 nm.

Next, lithium fluoride (LiF) was deposited on this second electron-transporting layer to form an electron-injecting electrode (cathode) having a thickness of 1 nm.

Then, on this electron-injecting electrode, metal A1 was deposited to form a metal A1 cathode having a thickness of 80 nm.

The device configuration of the organic EL device of Example 1 is schematically shown as follows.
ITO(130)/HI-1:HT-1(10; 3%)/HT-1(80)/EBL-1(5)/BH-1:BD-1(25; 4%)/HBL-1(5)/ET-1: Liq(20; 50 mass %)/LiF(1)/AI(80)

The numerical values in parentheses indicate the film thickness (unit: nm). Also, in parentheses, the number indicated in percentages represents the percentages (mass %) of the second compound in the first hole-injecting layer, the dopant material in the emitting layer, and the second compound in the second electron-transporting layer, respectively.

### (Evaluation of Organic EL Device)

Initial characteristics of the obtained organic EL devices were measured by driving at a constant current of 10 mA/cm² of DC (direct current) at room temperature. The measurement results of the driving voltage are shown in Table 1.

Furthermore, voltage was applied to the organic EL device to be 10 mA/cm² in current density, thereby measuring an EL emission spectrum by using Spectroradiometer CS-1000 (manufactured by KONICA MINOLTA, INC.). External quantum efficiency (EQE) (%) was calculated from the obtained spectral radiance spectrum. The results are shown in Table 2.

### Examples 2 to 3 and Comparative Example 1

The organic EL devices were fabricated and evaluated in the same manner as in Example 1, except that a compound ET-1 used in the second electron-transporting layer in Example 1 was replaced with a compound shown in Table 2 below. The results are shown in Table 2.

TABLE 2

| | | | | Affinity of |
|---|---|---|---|---|
| | External | | | material for the |
| | quantum | Driving | Material for the | second electron- |
| | efficiency | voltage | second electron- | transporting layer |
| | (%) | (V) | transporting layer | (eV) |
| Example 1 | 7.7 | 4.2 | ET-1 | 1.93 |
| Example 2 | 9.8 | 3.8 | ET-2 | 1.86 |
| Example 3 | 9.7 | 3.8 | ET-3 | 1.91 |
| Comp. Ex. 1 | 6.9 | 4.7 | Ref. ET-1 | 2.00 |

From the results shown in Table 2, it can be seen that the organic EL device of Example 1 using a compound ET-1 represented by the formula (1) has lower driving voltages and improved external quantum efficiencies as compared with the organic EL device of Comparative Example 1 using a compound Ref. ET-1 in which three 4-dibenzothiophenyl groups are substituted to a triazine ring in the second electron-transporting layer.

It can also be seen that the organic EL devices of Examples 2 and 3 using a compound ET-2 or ET-3 represented by the formula (A1) exhibits even lower driving voltages and higher external quantum efficiencies than the organic EL device using a compound ET-1. It is considered that compounds ET-2 and ET-3, which are materials of the second electron-transporting layer, have even lower affinity values than a compound ET-1 to enhance electron-injecting to the emitting layer, resulting in improved luminous efficiency and reduced driving voltages.

From the results shown in Table 1, it can be seen that a compound ET-1 represented by the formula (1) has a specifically lower Af value of 1.93 V compared to Ref. ET-1 and Ref. ET-2. This is an effect obtained by substituting a phenyl group at an ortho-position of a phenyl group bonded with a triazine ring.

Further, it can be seen that compounds ET-2 and ET-3 represented by the formula (A1) are even lower in Af value than a compound ET-1. This is an effect obtained by substituting a phenyl group at an ortho-position of a phenyl group bonded with a triazine ring and further substituting a fused ring on the triazine ring.

From the comparison in Tables 1 and 2, it is considered that the compound represented by the formula (A1), which includes the formula (1), has a low electron affinity (affinity) and a small difference in electron affinity with the host material or the first electron transport layer (hole blocking layer), thereby efficiently transporting electrons to the emitting layer. Therefore, the use of the compound represented by the formula (A1) as an electron-transporting material is considered to improve the electron-injecting property to the emitting layer, thereby improving the luminous efficiency of the organic EL device (EQE, external quantum efficiency).

### Examples 4 to 13 and Comparative Example 2

The organic EL devices were fabricated and evaluated in the same manner as in Example 1, except that a compound ET-1 used in the second electron-transporting layer in Example 1 was replaced with a compound shown in Table 3 below. The results are shown in Table 3 below together with Comparative Example 1 described above.

TABLE 3

| | External | Driving | Material for the |
|---|---|---|---|
| | quantum | voltage | second electron- |
| | efficiency (%) | (V) | transporting layer |
| Example 4 | 9.8 | 3.8 | ET-4 |
| Example 5 | 9.7 | 3.9 | ET-5 |
| Example 6 | 8.1 | 4.2 | ET-6 |
| Example 7 | 9.3 | 3.9 | ET-7 |
| Example 8 | 8.5 | 4.1 | ET-8 |
| Example 9 | 7.7 | 4.2 | ET-10 |
| Example 10 | 9.7 | 3.8 | ET-11 |
| Example 11 | 9.6 | 3.9 | ET-13 |
| Example 12 | 9.4 | 3.9 | ET-14 |
| Example 13 | 8.4 | 4.1 | ET-15 |
| Comp. Ex. 1 | 6.9 | 4.7 | Ref. ET-1 |
| Comp. Ex. 2 | 7.1 | 4.6 | Ref. ET-2 |

From the results shown in Table 1, it can be seen that compounds ET-4, ET-5, ET-7, ET-10, ET-11, ET-13, and ET-14 represented by the formula (A1) have specifically lower Af values of 1.86 to 1.93 V compared to Ref. ET-1 and Ref. ET-2. This is an effect obtained by substituting a phenyl group at an ortho-position of a phenyl group bonded with a triazine ring.

From the results of Table 3, the use of the compound represented by the formula (A1) as an electron-transporting material is considered to improve the electron-injecting property to the emitting layer, thereby improving the luminous efficiency of the organic EL device (EQE, external quantum efficiency).

### <Synthesis of Compounds>

### (Synthesis Example 1): Synthesis of Compound ET-1

Compound ET-1 was synthesized along the synthetic scheme below.

### (1) Synthesis of Intermediate A

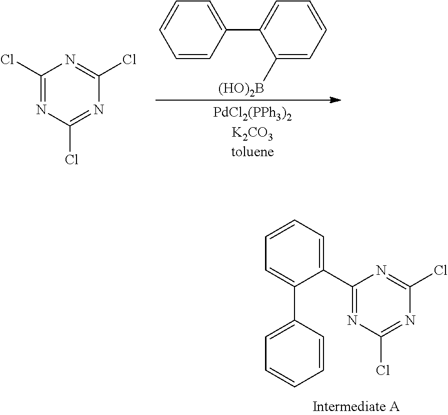

Cyanuric acid chloride (10 g) and biphenyl-2-boronic acid (7.2 g) were added in toluene (180 mL) and argon gas was passed through the resulting solution for 5 min. Dichlorobis (triphenylphosphine) palladium (0.13 g) and potassium carbonate (20 g) were added to this solution, and the solution was heated to 60° C. for 20 hours while stirring with under an argon atmosphere. The reaction solution was filtered to remove inorganic salts. The filtrate was subjected to column chromatography to obtain Intermediate A (2.3 g, 21% yield).

### (2) Synthesis of Compound ET-1

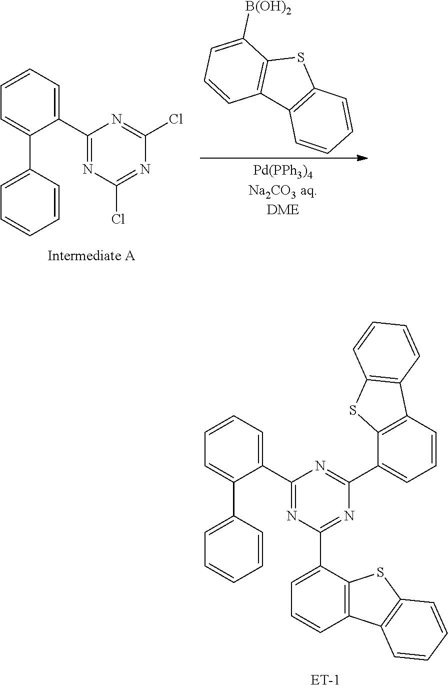 [SMILES: Clc1nc(Cl)nc(-c2ccccc2-c2ccccc2)n1 ; OB(O)c1cccc2c1sc1ccccc12 ; c1ccc(-c2ccccc2-c2nc(-c3cccc4c3sc3ccccc34)nc(-c3cccc4c3sc3ccccc34)n2)cc1]

Intermediate A (0.5 g) and benzothiophene-4-boronic acid (1.1 g) were added in dimethoxyethane (30 mL) and argon gas was passed through the resulting solution for 5 minutes. Tetrakis (triphenylphosphine) palladium (0.1 g) and an aqueous solution of sodium carbonate (2 M, 4 mL) were added to this solution, and the solution was heated and refluxed for 6 hours while stirring under an argon atmosphere. The reaction solution was filtered to obtain a solid. The solid was subjected to column chromatography to obtain the product (0.41 g, 41% yield). The molecular weight of ET-1 was 597.75, and the mass spectrum of the resulting compound was analyzed as m/z (ratio of mass to charge)=597, thereby identified as Compound ET-1.

### (Synthesis Example 2): Synthesis of Compound ET-2

Compound ET-2 was synthesized along the synthetic scheme below.

### (1) Synthesis of Intermediate B

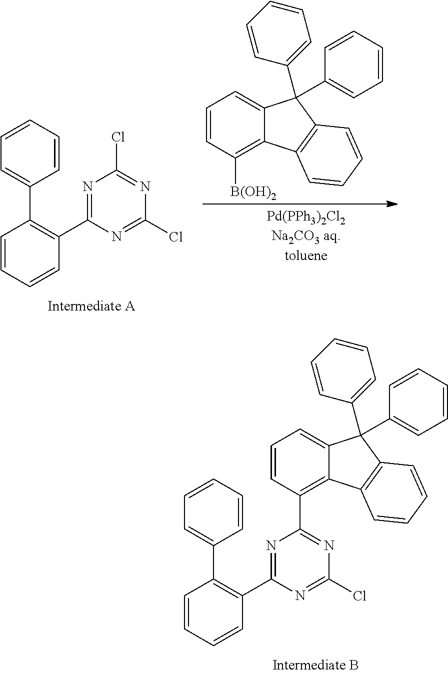 [SMILES: Clc1nc(Cl)nc(-c2ccccc2-c2ccccc2)n1 ; OB(O)c1cccc2c1-c1ccccc1C2(c1ccccc1)c1ccccc1 ; Clc1nc(-c2ccccc2-c2ccccc2)nc(-c2cccc3c2-c2ccccc2C3(c2ccccc2)c2ccccc2)n1]

Argon gas was passed through a solution of Intermediate A (7.2 g) and 9,9-diphenylfluorene-4-boronic acid (9.4 g) in toluene (120 mL) for 5 minutes. Dichlorobis (triphenylphosphine) palladium (83 mg) and an aqueous solution of sodium carbonate (2 M, 24 mL) was added to this solution and heated at 60° C. overnight while stirring under an argon atmosphere. The reaction solution was subjected to column chromatography after solvent distillation to obtain Intermediate B (8.3 g, 57% yield).

### (2) Synthesis of Compound ET-2

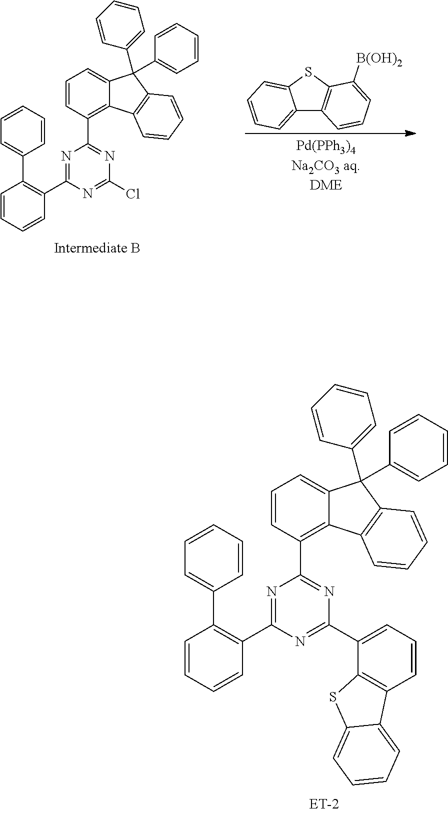 [SMILES: Clc1nc(-c2ccccc2-c2ccccc2)nc(-c2cccc3c2-c2ccccc2C3(c2ccccc2)c2ccccc2)n1 ; OB(O)c1cccc2c1sc1ccccc12 ; c1ccc(-c2ccccc2-c2nc(-c3cccc4c3-c3ccccc3C4(c3ccccc3)c3ccccc3)nc(-c3cccc4c3sc3ccccc34)n2)cc1]

Intermediate B (4.0 g) and dibenzothiophene-4-boronic acid (2.5 g) were dissolved in dimethoxyethane (DME) (70 mL) and argon gas was passed through the solution for 5 minutes. Tetrakis (triphenylphosphine) palladium (317 mg) and an aqueous solution of sodium carbonate (2 M, 10 mL) were added to the solution, and the solution was heated at 72° C. for 3 hours while stirring under an argon atmosphere. The reaction solution was subjected to column chromatography and the resulting solid was recrystallized with toluene to obtain ET-2 (2.8 g, 56% yield). The molecular weight of Compound ET-2 was 731.92, and the mass spectrum of the resulting compound was analyzed as m/z (ratio of mass to charge)=732, thereby identified as Compound ET-2.

### (Synthesis Example 3): Synthesis of Compound ET-3

Compound ET-3 was synthesized along the synthetic scheme below.

### (1) Synthesis of Intermediate C

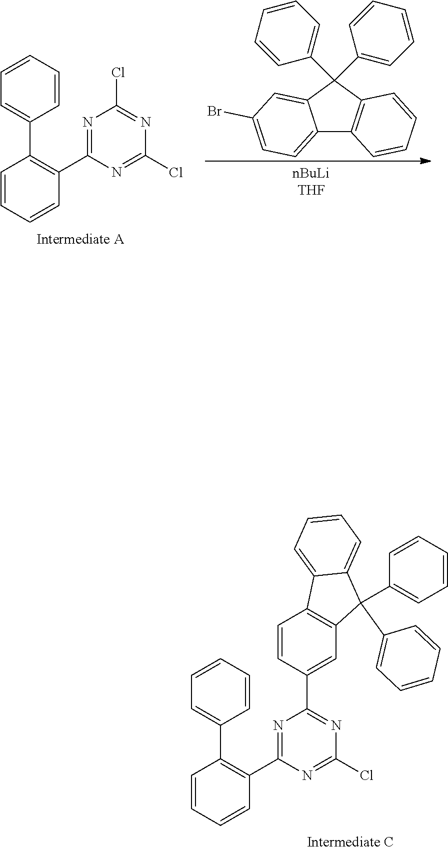 [SMILES: Clc1nc(Cl)nc(-c2ccccc2-c2ccccc2)n1 ; Brc1ccc2c(c1)C(c1ccccc1)(c1ccccc1)c1ccccc1-2 ; Clc1nc(-c2ccc3c(c2)C(c2ccccc2)(c2ccccc2)c2ccccc2-3)nc(-c2ccccc2-c2ccccc2)n1]

Under an argon atmosphere, a tetrahydrofuran solution (50 mL) of 9,9-diphenyl-2-bromo-fluorene (9.7 g) was cooled to −78° C., a n-hexanoic solution of normal butyllithium (1.6 M, 16 mL) was added dropwise to the solution over a period of 30 minutes, and then the solution was stirred at −78° C. for an additional 1 hour. This solution was added dropwise over 1 hour to a solution of Intermediate A (5.7 g) in tetrahydrofuran (100 mL) cooled to −78° C., followed by stirring at room temperature overnight. The solvent was distilled off from the reaction solution under reduced pressure to obtain a solid. This solid was subjected to column chromatography, and the resulting solid was washed with a mixed solvent of dichloromethane and hexane to obtain Intermediate C (7.6 g, yield 56%).

### (2) Synthesis of Compound ET-3

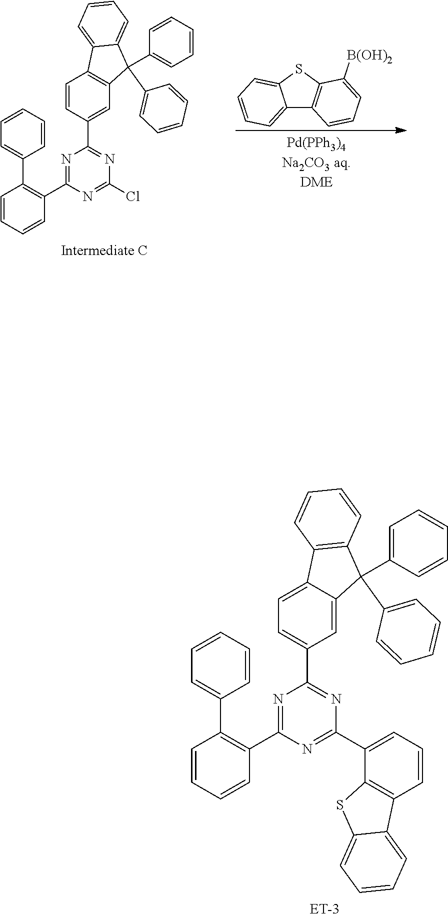 [SMILES: Clc1nc(-c2ccc3c(c2)C(c2ccccc2)(c2ccccc2)c2ccccc2-3)nc(-c2ccccc2-c2ccccc2)n1 ; OB(O)c1cccc2c1sc1ccccc12 ; c1ccc(-c2ccccc2-c2nc(-c3ccc4c(c3)C(c3ccccc3)(c3ccccc3)c3ccccc3-4)nc(-c3cccc4c3sc3ccccc34)n2)cc1]

Intermediate C (6.3 g) and dibenzothiophene-4-boronic acid (3.0 g) were dissolved in dimethoxyethane (DME) (100 mL) and argon gas was passed through the solution for 5 minutes. Tetrakis (triphenylphosphine) palladium (62 mg) and an aqueous solution of sodium carbonate (2 M, 17 mL) were added to the solution, and the solution was heated at 55° C. for 8 hours while stirring under an argon atmosphere. The solvent was distilled off from the reaction solution, and the obtained solid was washed with ethyl acetate, followed by recrystallization with a mixed solvent of hexane and toluene to obtain ET-3 (1.8 g, 23% yield). The molecular weight of Compound ET-3 was 731.97, and the mass spectrum of the resulting compound was analyzed as m/z (ratio of mass to charge)=732, thereby identified as Compound ET-3.

### (Synthesis Example 4): Synthesis of Compound ET-4

Compound ET-4 was synthesized along the synthetic scheme below.

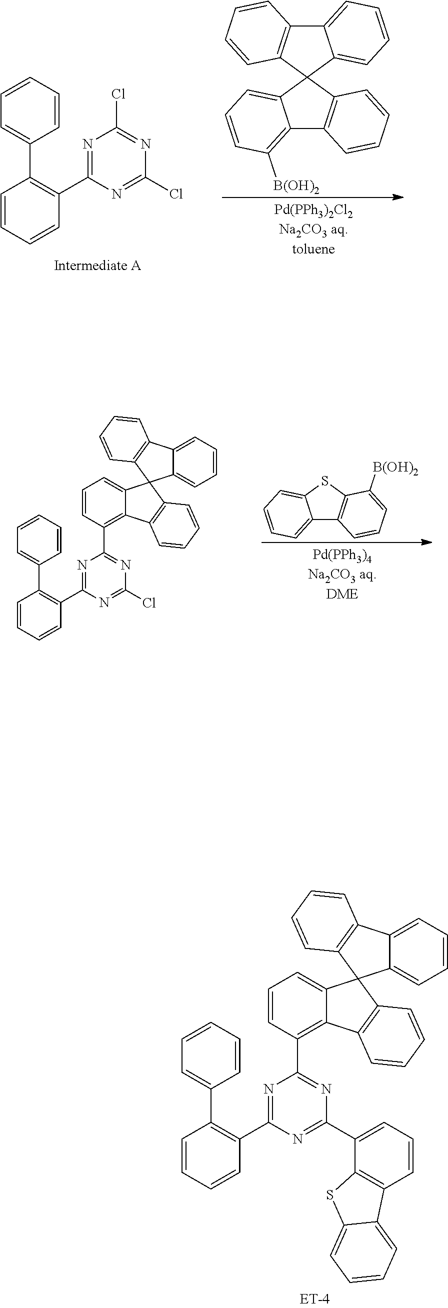 [SMILES: Clc1nc(Cl)nc(-c2ccccc2-c2ccccc2)n1 ; OB(O)c1cccc2c1-c1ccccc1C21c2ccccc2-c2ccccc21 ; Clc1nc(-c2ccccc2-c2ccccc2)nc(-c2cccc3c2-c2ccccc2C32c3ccccc3-c3ccccc32)n1 ; OB(O)c1cccc2c1sc1ccccc12 ; c1ccc(-c2ccccc2-c2nc(-c3cccc4c3-c3ccccc3C43c4ccccc4-c4ccccc43)nc(-c3cccc4c3sc3ccccc34)n2)cc1]

Compound ET-4 was obtained in the same manner as Compound ET-2, except that 9,9-diphenylfluorene-4-boronic acid was changed to 9,9′-spirobifluorene-4-boronic acid. The molecular weight of Compound ET-4 was 729.90, and the mass spectrum of the resulting compound was analyzed as m/z (ratio of mass to charge)=730, thereby identified as Compound ET-4.

### (Synthesis Example 5): Synthesis of Compound ET-5

Compound ET-5 was synthesized along the synthetic scheme below.

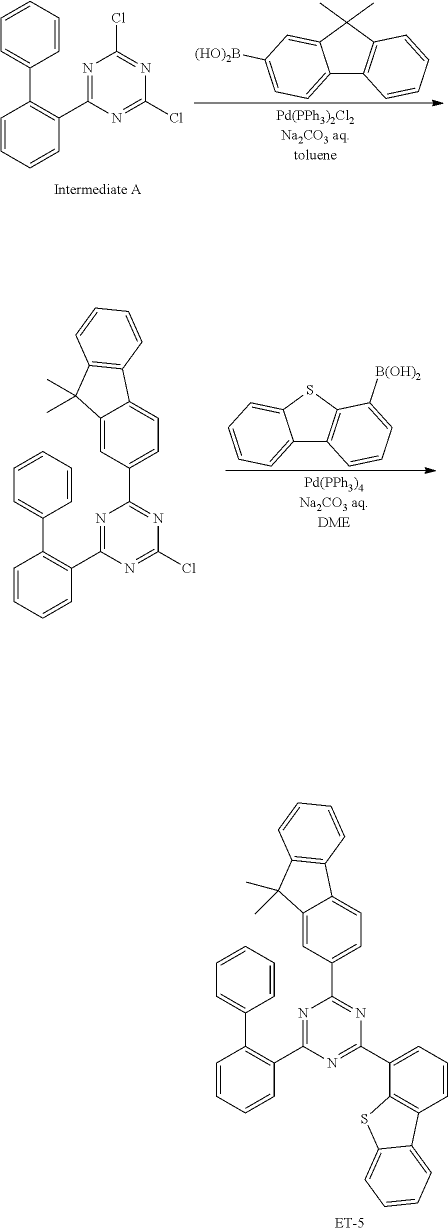 [SMILES: Clc1nc(Cl)nc(-c2ccccc2-c2ccccc2)n1 ; CC1(C)c2ccccc2-c2ccc(B(O)O)cc21 ; CC1(C)c2ccccc2-c2ccc(-c3nc(Cl)nc(-c4ccccc4-c4ccccc4)n3)cc21 ; OB(O)c1cccc2c1sc1ccccc12 ; CC1(C)c2ccccc2-c2ccc(-c3nc(-c4ccccc4-c4ccccc4)nc(-c4cccc5c4sc4ccccc45)n3)cc21]

Compound ET-5 was obtained in the same manner as Compound ET-2, except that 9,9-diphenylfluorene-4-boronic acid was changed to 9,9-dimethylfluorene-2-boronic acid. The molecular weight of Compound ET-5 was 607.78, and the mass spectrum of the resulting compound was analyzed as m/z (ratio of mass to charge)=608, thereby identified as Compound ET-5.

### (Synthesis Example 6): Synthesis of Compound ET-6

Compound ET-6 was synthesized along the synthetic scheme below.

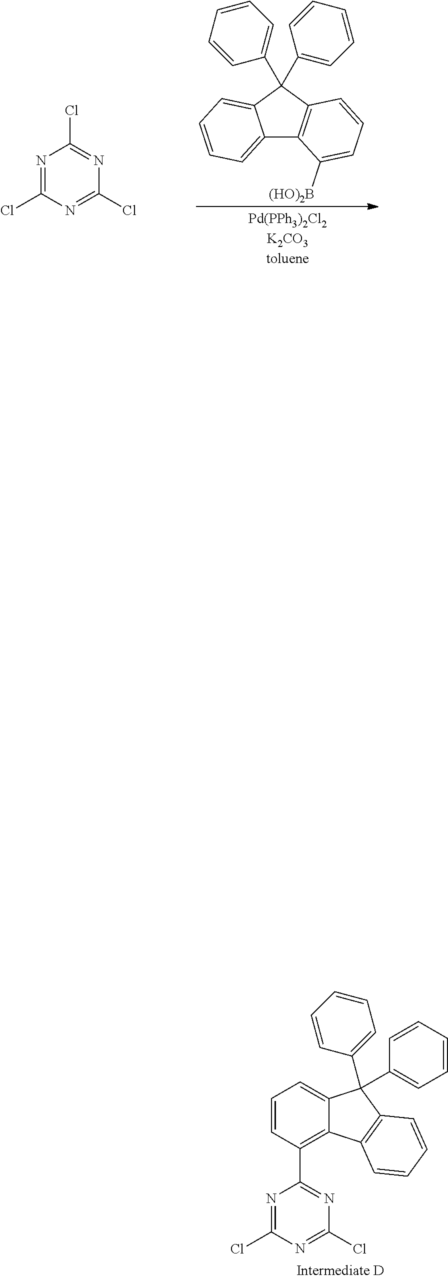 [SMILES: Clc1nc(Cl)nc(Cl)n1 ; OB(O)c1cccc2c1-c1ccccc1C2(c1ccccc1)c1ccccc1 ; Clc1nc(Cl)nc(-c2cccc3c2-c2ccccc2C3(c2ccccc2)c2ccccc2)n1]

Cyanuric acid chloride (10 g) and 9,9-diphenylfluorene-4-boronic acid (14.1 g) were added in toluene (150 mL) and argon gas was passed through the resulting solution for 5 minutes. Dichlorobis (triphenylphosphine) palladium (0.14 g) and potassium carbonate (16 g) were added to this solution, and the solution was heated at 60° C. for 15 hours while stirring under an argon atmosphere. The reaction solution was filtered to remove inorganic salts. The filtrate was subjected to column chromatography to obtain Intermediate D (5.5 g, 30% yield).

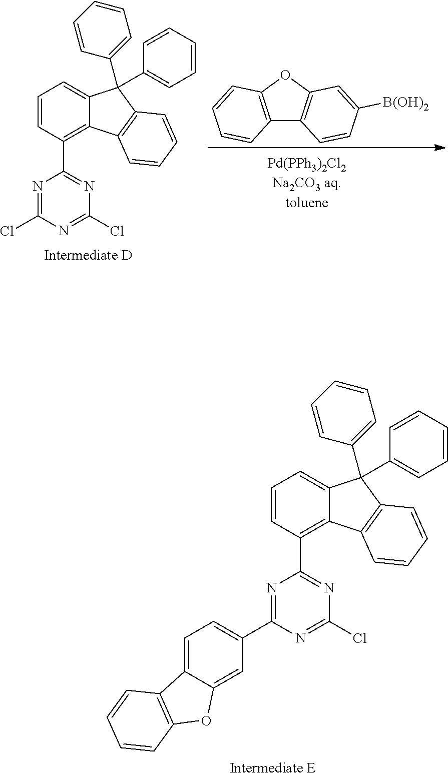

Intermediate D (5.5 g) and dibenzofuran-3-boronic acid (2.5 g) were dissolved in toluene (120 mL) and argon gas was passed through the solution for 5 minutes. Dichlorobis (triphenylphosphine) palladium (83 mg) and an aqueous solution of sodium carbonate (2 M, 18 mL) was added to this solution, and the solution was heated at 55° C. for 10 hours while stirring under an argon atmosphere. The solvent was distilled off from the reaction solution, and the obtained solid was washed with ethyl acetate, followed by recrystallization with a mixed solvent of hexane and toluene to obtain Intermediate E (2.1 g, 30% yield).

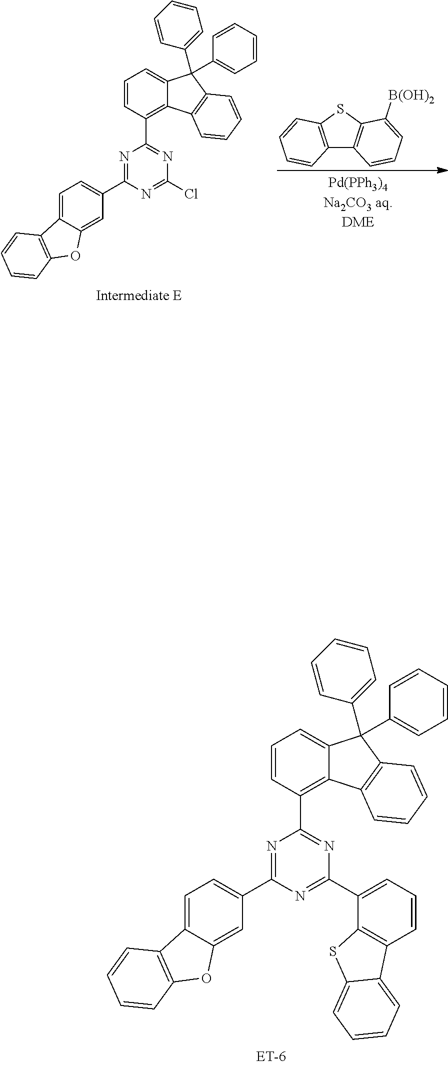 [SMILES: Clc1nc(-c2ccc3c(c2)oc2ccccc23)nc(-c2cccc3c2-c2ccccc2C3(c2ccccc2)c2ccccc2)n1 ; OB(O)c1cccc2c1sc1ccccc12 ; c1ccc(C2(c3ccccc3)c3ccccc3-c3c(-c4nc(-c5ccc6c(c5)oc5ccccc56)nc(-c5cccc6c5sc5ccccc56)n4)cccc32)cc1]

Intermediate E (3.0 g) and dibenzothiophene-4-boronic acid (1.1 g) were dissolved in dimethoxyethane (DME) (100 mL) and argon gas was passed through the solution for 5 minutes. Tetrakis (triphenylphosphine) palladium (58 mg) and an aqueous solution of sodium carbonate (2 M, 7.5 mL) were added to the solution, and the solution was heated at 55° C. for 9 hours while stirring under an argon atmosphere. The solvent was distilled off from the reaction solution, and the obtained solid was washed with ethyl acetate, followed by recrystallization with toluene to obtain ET-6 (1.2 g, 31% yield). The molecular weight of Compound ET-6 was 745.90, and the mass spectrum of the resulting compound was analyzed as m/z (ratio of mass to charge)=746, thereby identified as Compound ET-6.

### (Synthesis Example 7): Synthesis of Compound ET-7

Compound ET-7 was synthesized along the synthetic scheme below.

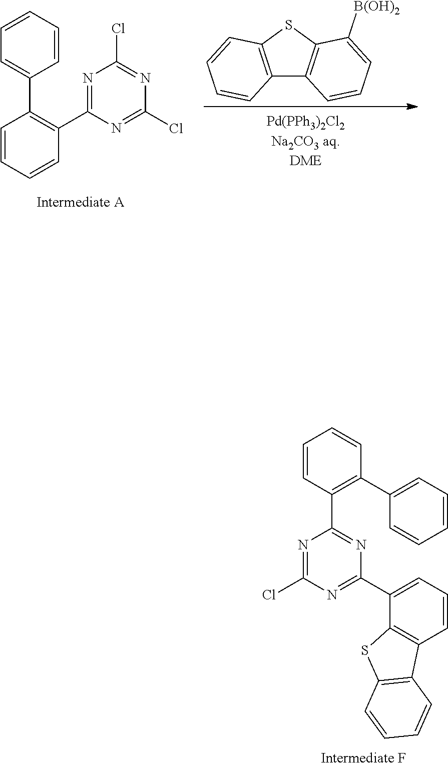 [SMILES: Clc1nc(Cl)nc(-c2ccccc2-c2ccccc2)n1 ; OB(O)c1cccc2c1sc1ccccc12 ; Clc1nc(-c2ccccc2-c2ccccc2)nc(-c2cccc3c2sc2ccccc23)n1]

Intermediate A (2.5 g) and dibenzothiophene-4-boronic acid (1.9 g) were dissolved in toluene (100 mL) and argon gas was passed through the solution for 5 minutes. Dichlorobis (triphenylphosphine) palladium (116 mg) and an aqueous solution of sodium carbonate (2 M, 12 mL) was added to this solution, and the solution was heated at 55° C. for 10 hours while stirring under an argon atmosphere. The solvent was distilled off from the reaction solution, and the resulting solid was subjected to column chromatography to obtain Intermediate F (0.7 g, 19% yield).

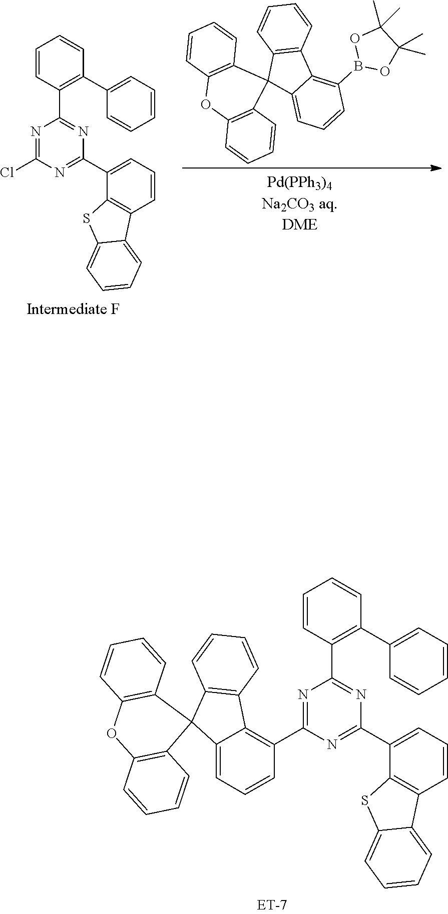 [SMILES: Clc1nc(-c2ccccc2-c2ccccc2)nc(-c2cccc3c2sc2ccccc23)n1 ; CC1(C)OB(c2cccc3c2-c2ccccc2C32c3ccccc3Oc3ccccc32)OC1(C)C ; c1ccc(-c2ccccc2-c2nc(-c3cccc4c3-c3ccccc3C43c4ccccc4Oc4ccccc43)nc(-c3cccc4c3sc3ccccc34)n2)cc1]

Intermediate F (2.3 g) and spiro[9H-fluorene-9,9′-[9H]xanthene]-4-(4,4,5,5-tetramethyl)-1,3,2-dioxaborolane (synthesized according to WO2014/072017A1) (2.3 g) were dissolved in dimethoxyethane (DME) (100 mL) and argon gas was passed through the solution for 5 minutes. Tetrakis (triphenylphosphine) palladium (59 mg) and an aqueous solution of sodium carbonate (2 M, 15 mL) were added to the solution, and the solution was heated at 55° C. for 9 hours while stirring under an argon atmosphere. The solvent was distilled off from the reaction solution, and the resulting solid was subjected to column chromatography to obtain a ET-7 (2.0 g, 53% yield). The molecular weight of Compound ET-7 was 745.90, and the mass spectrum of the resulting compound was analyzed as m/z (ratio of mass to charge)=746, thereby identified as Compound ET-7.

### (Synthesis Example 8): Synthesis of Compound ET-8

Compound ET-8 was synthesized along the synthetic scheme below.

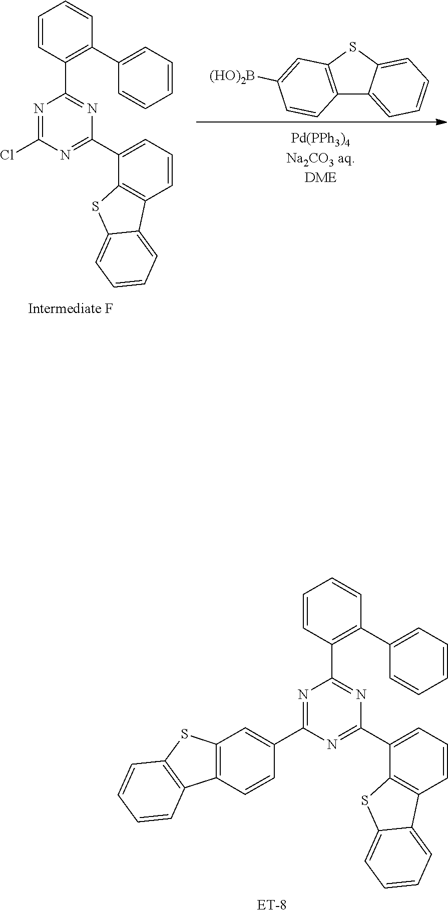 [SMILES: Clc1nc(-c2ccccc2-c2ccccc2)nc(-c2cccc3c2sc2ccccc23)n1 ; OB(O)c1ccc2c(c1)sc1ccccc12 ; c1ccc(-c2ccccc2-c2nc(-c3ccc4c(c3)sc3ccccc34)nc(-c3cccc4c3sc3ccccc34)n2)cc1]

Intermediate F (3.0 g) and dibenzothiophene-3-boronic acid (1.5 g) were dissolved in dimethoxyethane (DME) (150 mL) and argon gas was passed through the solution for 5 minutes. Tetrakis (triphenylphosphine) palladium (154 mg) and an aqueous solution of sodium carbonate (2 M, 10 mL) were added to the solution, and the solution was heated at 55° C. for 6 hours while stirring under an argon atmosphere. The solvent was distilled off from the reaction solution, and the resulting solid was subjected to column chromatography to obtain ET-8 (2.4 g, 60% yield). The molecular weight of Compound ET-8 was 597.75, and the mass spectrum of the resulting compound was analyzed as m/z (ratio of mass to charge)=598, thereby identified as Compound ET-8.

### (Synthesis Example 9): Synthesis of Compound ET-9

Compound ET-9 was synthesized along the synthetic scheme below.

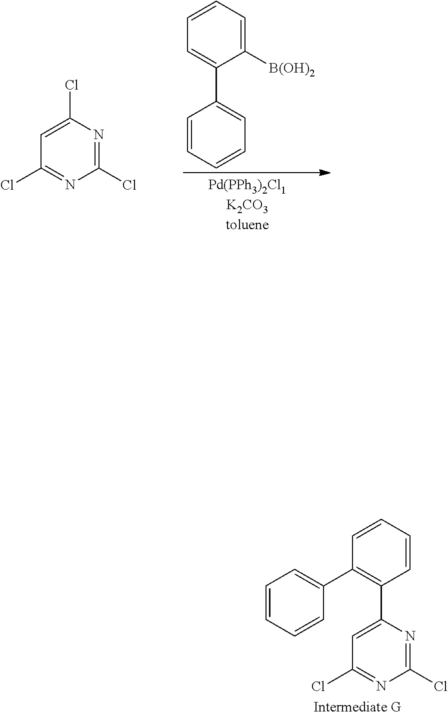 [SMILES: Clc1cc(Cl)nc(Cl)n1 ; OB(O)c1ccccc1-c1ccccc1 ; Clc1cc(-c2ccccc2-c2ccccc2)nc(Cl)n1]

2,4,6-trichloropyrimidine (5.0 g) and 2-biphenylboronic acid (5.4 g) were added in toluene (250 mL) and argon gas was passed through the resulting solution for 5 minutes. Dichlorobis (triphenylphosphine) palladium (0.1 g) and potassium carbonate (11 g) were added to this solution, and the solution was heated at 60° C. for 10 hours while stirring under an argon atmosphere. The reaction solution was filtered to remove inorganic salts. After evaporation of the filtrate in vacuo, the residue was subjected to column chromatography to obtain Intermediate G (3.3 g, 40% yield).

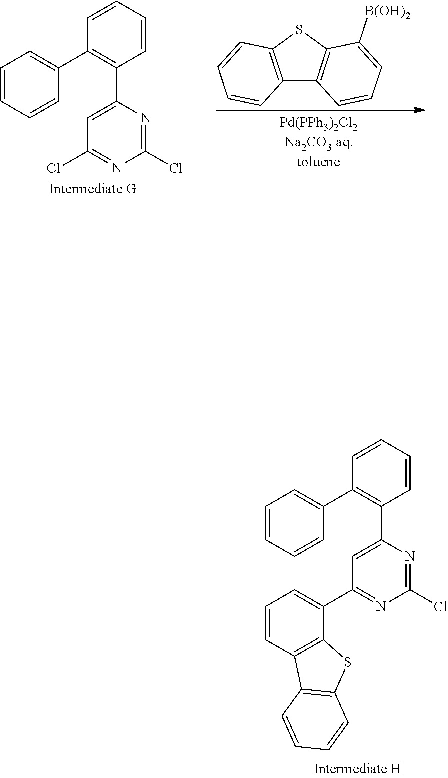 [SMILES: Clc1cc(-c2ccccc2-c2ccccc2)nc(Cl)n1 ; OB(O)c1cccc2c1sc1ccccc12 ; Clc1nc(-c2ccccc2-c2ccccc2)cc(-c2cccc3c2sc2ccccc23)n1]

Intermediate G (3.3 g) and dibenzothiophen-4-boronic acid (2.5 g) were dissolved in toluene (100 mL) and argon gas was passed through the solution for 5 minutes. Dichlorobis (triphenylphosphine) palladium (38 mg) and an aqueous solution of sodium carbonate (2 M, 11 mL) was added to this solution, and the solution was heated at 50° C. for 8 hours while stirring under an argon atmosphere. The solvent was distilled off from the reaction solution, and the resulting solid was subjected to column chromatography to obtain Intermediate H (1.7 g, 35% yield).

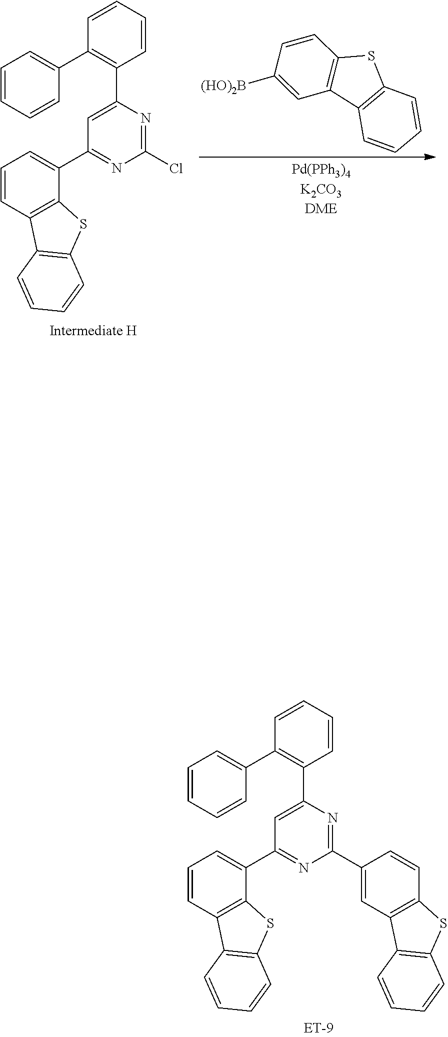 [SMILES: Clc1nc(-c2ccccc2-c2ccccc2)cc(-c2cccc3c2sc2ccccc23)n1 ; OB(O)c1ccc2sc3ccccc3c2c1 ; c1ccc(-c2ccccc2-c2cc(-c3cccc4c3sc3ccccc34)nc(-c3ccc4sc5ccccc5c4c3)n2)cc1]

Intermediate H (4.0 g) and dibenzothiophene-2-boronic acid (2.0 g) were dissolved in dimethoxyethane (DME) (100 mL) and argon gas was passed through the solution for 5 minutes. Tetrakis (triphenylphosphine) palladium (103 mg) and potassium carbonate (3.7 g) were added to this solution, and the solution was heated at 55° C. for 8 hours while stirring under an argon atmosphere. The solvent was distilled off from the reaction solution, and the resulting solid was recrystallized with toluene to obtain ET-9 (3.6 g, 67% yield). The molecular weight of Compound ET-9 was 596.77, and the mass spectrum of the resulting compound was analyzed as m/z (ratio of mass to charge)=597, thereby identified as Compound ET-9.

### (Synthesis Example 10): Synthesis of Compound ET-10

Compound ET-10 was synthesized along the synthetic scheme below.

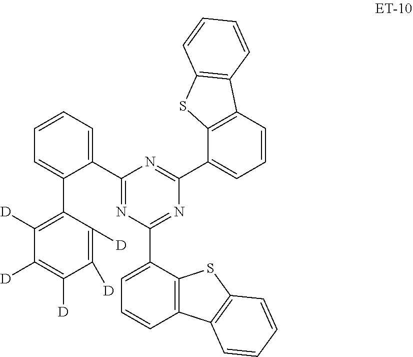 [SMILES: [2H]c1c([2H])c([2H])c(-c2ccccc2-c2nc(-c3cccc4c3sc3ccccc34)nc(-c3cccc4c3sc3ccccc34)n2)c([2H])c1[2H]]

Compound ET-10 was synthesized by the same synthetic scheme as Synthesis Example 1, except that 1,1′-biphenyl-2′-boronic acid (2,3,4,5,6-d) was used instead of biphenyl-2-boronic acid.

The molecular weight of Compound ET-10 was 602.78, and the mass spectrum of the resulting compound was analyzed as m/z (ratio of mass to charge)=602, thereby the obtained product was identified as Compound ET-10.

### (Synthesis Example 11): Synthesis of Compound ET-11

Compound ET-11 was synthesized along the synthetic scheme below.

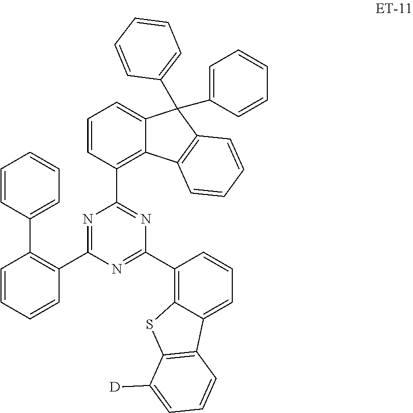 [SMILES: [2H]c1cccc2c1sc1c(-c3nc(-c4ccccc4-c4ccccc4)nc(-c4cccc5c4-c4ccccc4C5(c4ccccc4)c4ccccc4)n3)cccc12]

Compound ET-11 was synthesized by the same synthetic scheme as Synthesis Example 2, except that dibenzothiophen-4-boronic acid (6-d) was used instead of dibenzothiophen-4-boronic acid.

The molecular weight of Compound ET-11 was 732.92, and the mass spectrum of the resulting compound was analyzed as m/z (ratio of mass to charge)=732, thereby the obtained product was identified as Compound ET-11.

### (Synthesis Example 12): Synthesis of Compound ET-12

Compound ET-12 was synthesized along the synthetic scheme below.

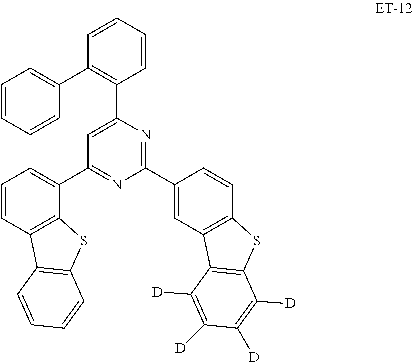 [SMILES: [2H]c1c([2H])c([2H])c2c(sc3ccc(-c4nc(-c5ccccc5-c5ccccc5)cc(-c5cccc6c5sc5ccccc56)n4)cc32)c1[2H]]

Compound ET-12 was synthesized by the same synthetic scheme in Synthesis Example 9, except that deuterated dibenzothiophene-2-boronic acid (6,7,8,9-d) was used instead of dibenzothiophene-2-boronic acid.

The molecular weight of Compound ET-12 was 600.79, and the mass spectrum of the resulting compound was analyzed as m/z (ratio of mass to charge)=600, thereby the obtained product was identified as Compound ET-12.

### (Synthesis Example 13): Synthesis of Compound ET-13

Compound ET-13 was synthesized along the synthetic scheme below.

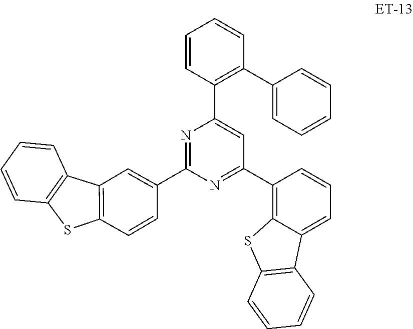 [SMILES: c1ccc(-c2ccccc2-c2cc(-c3cccc4c3sc3ccccc34)nc(-c3ccc4sc5ccccc5c4c3)n2)cc1]

Compound ET-13 was synthesized by the same synthetic scheme in Synthesis Example 8, except that deuterated dibenzothiophene-2-boronic acid was used instead of dibenzothiophene-3-boronic acid.

The molecular weight of Compound ET-13 was 597.75, and the mass spectrum of the resulting compound was analyzed as m/z (ratio of mass to charge)=597, thereby the obtained product was identified as Compound ET-13.

### (Synthesis Example 14): Synthesis of Compound ET-14

Compound ET-14 was synthesized along the synthetic scheme below.

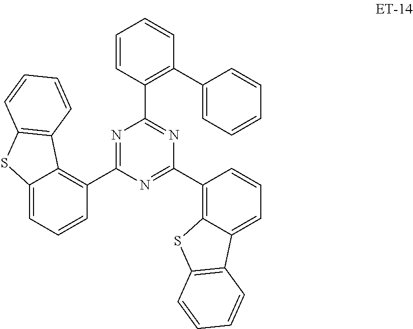 [SMILES: c1ccc(-c2ccccc2-c2nc(-c3cccc4c3sc3ccccc34)nc(-c3cccc4sc5ccccc5c34)n2)cc1]

Compound ET-14 was synthesized by the same synthetic scheme in Synthesis Example 8, except that deuterated dibenzothiophene-1-boronic acid was used instead of dibenzothiophene-3-boronic acid.

The molecular weight of Compound ET-14 was 597.75, and the mass spectrum of the resulting compound was analyzed as m/z (ratio of mass to charge)=597, thereby the obtained product was identified as Compound ET-14.

### (Synthesis Example 15): Synthesis of Compound ET-15

Compound ET-15 was synthesized along the synthetic scheme below.

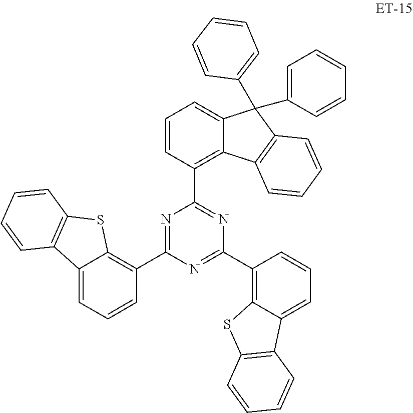 [SMILES: c1ccc(C2(c3ccccc3)c3ccccc3-c3c(-c4nc(-c5cccc6c5sc5ccccc56)nc(-c5cccc6c5sc5ccccc56)n4)cccc32)cc1]

Compound ET-15 was synthesized by the same synthetic scheme as Synthesis Example 1, except that 9,9-diphenylfluorene-4-boronic acid was used instead of biphenyl-2-boronic acid.

The molecular weight of Compound ET-15 was 761.96, and the mass spectrum of the resulting compound was analyzed as m/z (ratio of mass to charge)=761, thereby the obtained product was identified as Compound ET-15.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

1-40. (canceled)

41. A compound represented by the following formula (A1): 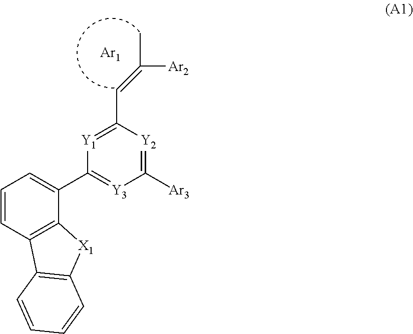
wherein in the formula (A1),
X₁ is O or S;
Y₁, Y₂, and Y₃ are independently CH or N;
provided that two or more of Y₁, Y₂, and Y₃ are N;
Ar₁ is an aryl group including 6 to 50 ring carbon atoms, comprising a benzene ring having at least substituent Ar₂ at the ortho-position; Ar₁ may have one or more substituents in addition to Ar₂;
Ar₂ is a substituted or unsubstituted aryl group including 6 to 50 ring carbon atoms;
Ar₁ and Ar₂ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring;
Ar₃ is a group represented by the following formula (A2-1: 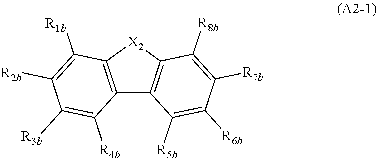
wherein in the formula (A2-1),
X₂ is O or S;
one of R_{1b} to R_{8b} is a single bond bonding with the carbon atom between Y₂ and Y₃, and the others are hydrogen atoms.

42. The compound according to claim 41, wherein the compound represented by the formula (A1) is a compound represented by the following formula (A7): 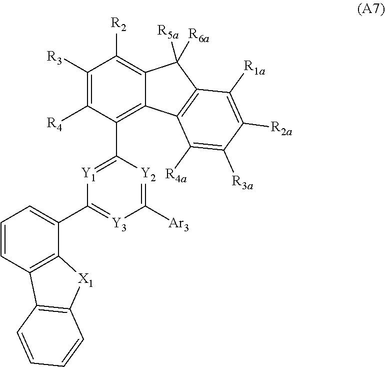
wherein in the formula (A7), X₁, Y₁ to Y₃, and Ar₃ are as defined in formula (A1);
R₅ₐ and R₆ₐ form a substituted or unsubstituted, saturated or unsaturated ring by bonding with each other, or do not form a substituted or unsubstituted, saturated or unsaturated ring;
R₅ₐ and R₆ₐ which do not form the substituted or unsubstituted, saturated or unsaturated ring are independently
a hydrogen atom,
a substituted or unsubstituted alkyl group including 1 to 50 carbon atoms, or
a substituted or unsubstituted aryl group including 6 to 50 ring carbon atoms; and
one or more sets of adjacent two or more of R₂ to R₄ and one or more sets of adjacent two or more of R₁ₐ to R₄ₐ form a substituted or unsubstituted, saturated or unsaturated ring by bonding with each other, or do not form a substituted or unsubstituted, saturated or unsaturated ring;
R₂ to R₄ and R₁ₐ to R₄ₐ which do not form the substituted or unsubstituted, saturated or unsaturated ring are independently
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

43. The compound according to claim 41, wherein Y₁ to Y₃ are N.

44. The compound according to claim 41, wherein X₁ is S.

45. The compound according to claim 41, wherein the compound represented by the formula (A1) is a compound represented by the following formula (1): 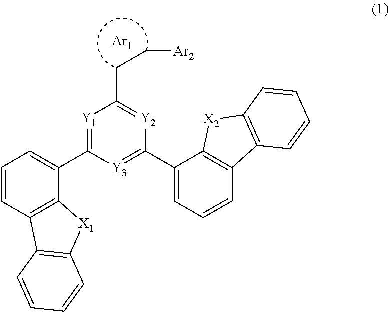
wherein in the formula (1),
X₁ and X₂ are independently O or S;
Y₁, Y₂, and Y₃ are independently CH or N;
provided that two or more of Y₁, Y₂, and Y₃ are N;
Ar₁ is an aryl group including 6 to 50 ring carbon atoms, comprising a benzene ring substituted by Ar₂ at least in the ortho-position; Ar₁ may have a substituent in addition to Ar₂;
Ar₂ is a substituted or unsubstituted aryl group including 6 to 50 ring carbon atoms;
Ar₁ and Ar₂ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring.

46. The compound according to claim 45, wherein the compound represented by the formula (1) is a compound represented by the following formula (2): 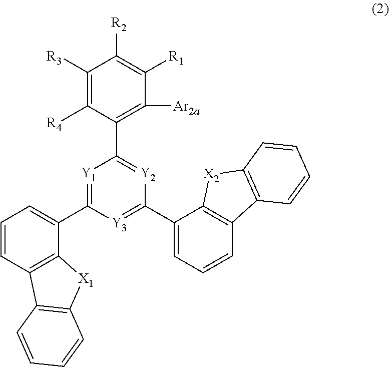
wherein in the formula (2), X₁, X₂, and Y₁ to Y₃ are as defined in the formula (1);
Ar₂ₐ is a substituted or unsubstituted aryl group including 6 to 50 ring carbon atoms;
Ar₂ₐ, which is the aryl group, and R₁ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring;
one or more sets of adjacent two or more of R₁, which does not form the substituted or unsubstituted 5-membered hydrocarbon ring by bonding with Ar₂ₐ, and R₂ to R₄ form a substituted or unsubstituted, saturated or unsaturated ring or do not form a substituted or unsubstituted, saturated or unsaturated ring;
R₁ which does not form the substituted or unsubstituted 5-membered hydrocarbon ring by bonding with Ar₂ₐ and does not form the substituted or unsubstituted, saturated or unsaturated ring, and R₂ to R₄ which do not form the substituted or unsubstituted, saturated or unsaturated ring are independently
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

47. The compound according to claim 45, wherein the compound represented by the formula (1) is a compound represented by the following formula (2H): 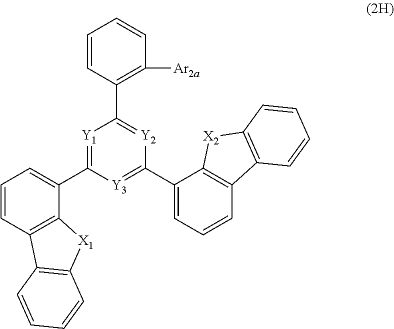
wherein in the formula (2H), X₁, X₂, and Y₁ to Y₃ are as defined in formula (1);
Ar₂ₐ is a substituted or unsubstituted aryl group including 6 to 50 ring carbon atoms.

48. The compound according to claim 45, wherein the compound represented by the formula (1) is a compound represented by the following formula (3-1) or a compound represented by the following formula (3-2): 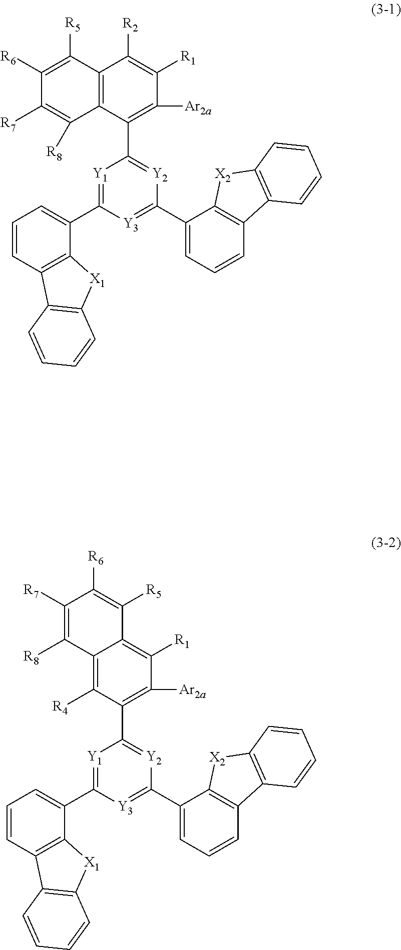
wherein in the formulas (3-1) and (3-2), X₁, X₂, and Y₁ to Y₃ are as defined in the formula (1);
Ar₂ₐ is a substituted or unsubstituted aryl group including 6 to 50 ring carbon atoms;
Ar₂ₐ, which is the aryl group, and R₁ form a substituted or unsubstituted 5-membered hydrocarbon ring to form a polycyclic fused aryl group fused via the 5-membered hydrocarbon ring by bonding with each other, or do not form a substituted or unsubstituted 5-membered hydrocarbon ring;
one or more sets of adjacent two or more of R₁, which does not form the substituted or unsubstituted 5-membered hydrocarbon ring by bonding with Ar₂ₐ, and R₂ and R₄ to R₈ form a substituted or unsubstituted, saturated or unsaturated ring or do not form a substituted or unsubstituted, saturated or unsaturated ring;
R₁ which does not form the substituted or unsubstituted 5-membered hydrocarbon ring by bonding with Ar₂ₐ and does not form the substituted or unsubstituted, saturated or unsaturated ring, and R₂ and R₄ to R₈ which do not form the substituted or unsubstituted, saturated or unsaturated ring are independently
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

49. The compound according to claim 45, wherein the compound represented by the formula (1) is a compound represented by the following formula (3H-1) or a compound represented by the following formula (3H-2): 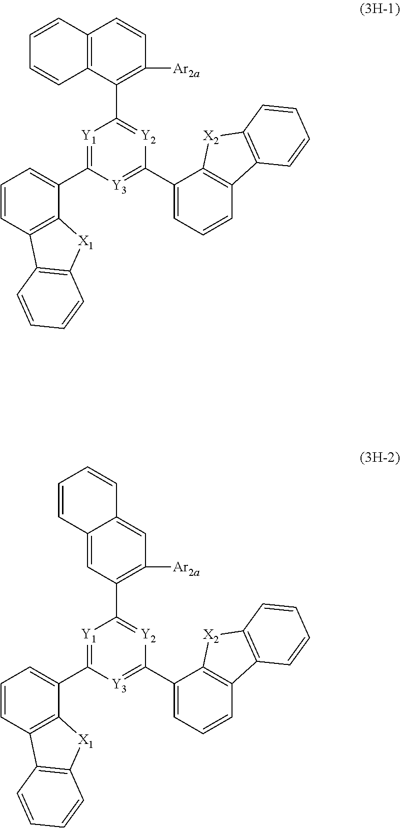
wherein in the formulas (3H-1) and (3H-2), X₁, X₂, and Y₁ to Y₃ are as defined in formula (1);
Ar₂ₐ is a substituted or unsubstituted aryl group including 6 to 50 ring carbon atoms.

50. The compound according to claim 45, wherein the compound represented by the formula (1) is a compound represented by the following formula (4): 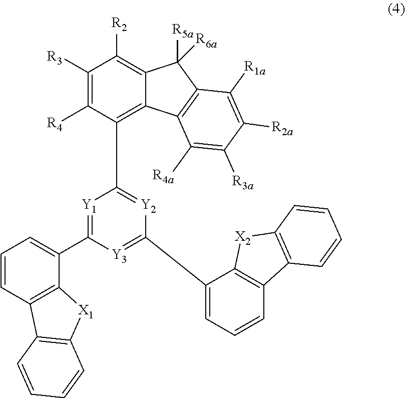
wherein in the formula (4), X₁, X₂, and Y₁ to Y₃ are as defined in formula (1);
R₅ₐ and R₆ₐ form a substituted or unsubstituted, saturated or unsaturated ring by bonding with each other, or do not form a substituted or unsubstituted, saturated or unsaturated ring;
R₅ₐ and R₆ₐ which do not form the substituted or unsubstituted, saturated or unsaturated ring are independently
a hydrogen atom,
a substituted or unsubstituted alkyl group including 1 to 50 carbon atoms, or
a substituted or unsubstituted aryl group including 6 to 50 ring carbon atoms; and
one or more sets of adjacent two or more of R₂ to R₄ and one or more sets of adjacent two or more of R₁ₐ to R₄ₐ form a substituted or unsubstituted, saturated or unsaturated ring by bonding with each other, or do not form a substituted or unsubstituted, saturated or unsaturated ring;
R₂ to R₄ and R₁ₐ to R₄ₐ which do not form the substituted or unsubstituted, saturated or unsaturated ring are independently
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom,
a cyano group,
a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ are the same or different.

51. The compound according to claim 45, wherein the compound represented by the formula (1) is a compound represented by the following formula (4H): 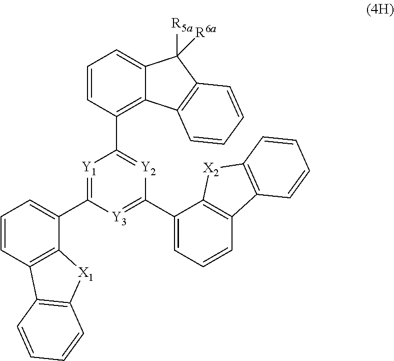
wherein in the formula (4H), X₁, X₂, and Y₁ to Y₃ are as defined in formula (1);
R₅ₐ and R₆ₐ form a substituted or unsubstituted, saturated or unsaturated ring by bonding with each other, or do not form a substituted or unsubstituted, saturated or unsaturated ring; and
R₅ₐ and R₆ₐ which do not form the substituted or unsubstituted, saturated or unsaturated ring are independently
a hydrogen atom,
a substituted or unsubstituted alkyl group including 1 to 50 carbon atoms, or
a substituted or unsubstituted aryl group including 6 to 50 ring carbon atoms.

52. The compound according to claim 46, wherein Ar₂ₐ is
a substituted or unsubstituted phenyl group,
a substituted or unsubstituted naphthyl group,
a substituted or unsubstituted anthryl group, or
a substituted or unsubstituted biphenyl group.

53. The compound according to claim 41, wherein the substituent of the “substituted or unsubstituted” is
a substituted or unsubstituted alkyl group including 1 to 50 carbon atoms, or
a substituted or unsubstituted aryl group including 6 to 50 ring carbon atoms.

54. The compound according to claim 41, wherein one of X₁ and X₂ is S and the other is O.

55. The compound according to claim 45, wherein both X₁ and X₂ are S.

56. The compound according to claim 41, wherein all of Y₁ to Y₃ are N.

57. An electron-transporting material for an organic electroluminescence device, comprising the compound according to claim 41.

58. An organic electroluminescence device comprising an anode, an organic layer, and a cathode in this order, wherein
the organic layer comprises the compound according to claim 41.

59. An organic electroluminescence device comprising an anode, an organic layer, and a cathode in this order, wherein
the organic layer comprises the compound according to claim 41, and wherein the compound according to claim 41 has at least one deuterium atom.

60. An organic electroluminescence device comprising an anode, an emitting layer, an electron-transporting region, and a cathode in this order, wherein
the electron transporting region comprises the compound according to claim 41.

61. An organic electroluminescence device comprising an anode, an emitting layer, an electron-transporting region, and a cathode in this order, wherein
the electron transporting region comprises the compound according to claim 41,
the electron-transporting region comprises a first electron-transporting layer, and a second electron-transporting layer, and the emitting layer, the first electron-transporting layer, the second electron-transporting layer and the cathode in this order, and
at least one of the first electron-transporting layer and the second electron-transporting layer comprises the compound according to claim 41.

62. The organic electroluminescence device according to claim 60, wherein,
the emitting layer comprises the compound represented by the following formula (11): 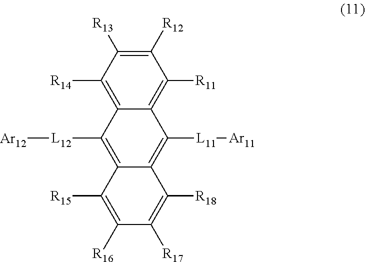
wherein in the formula (11),
R₁₁ to R₁₈ are independently
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃)
—O—(R₉₀₄)
—S—(R₉₀₅)
—N(R₉₀₆)(R₉₀₇)
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms;
when two or more of each of R₉₀₁ to R₉₀₇ are present, two or more of each of R₉₀₁ to R₉₀₇ may be the same as or different to each other;
adjacent two or more of R₁₁ to R₁₄, and adjacent two or more R₁₅ to R₁₈ do not form a ring by bonding with each other;
L₁₁ and L₁₂ are independently
a single bond,
a substituted or unsubstituted arylene group including 6 to 50 ring carbon atoms, or
a substituted or unsubstituted divalent heterocyclic ring including 5 to 50 ring atoms; and
Ar₁₁ and Ar₁₂ are independently
a substituted or unsubstituted aryl group including 6 to 50 ring carbon atoms, or
a substituted or unsubstituted monovalent heterocyclic group including 5 to 50 ring atoms.

63. The organic electroluminescence device according to claim 62, wherein the compound represented by the formula (11) is the compound represented by the following formula (12): 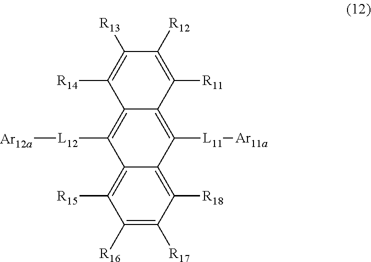
wherein in the formula (12), R₁₁ to R₁₈, L₁₁ and L₁₂ are as defined in the formula (11);
at least one of Ar₁₁ₐ and Ar₁₂ₐ is the monovalent group represented by the following formula (20): 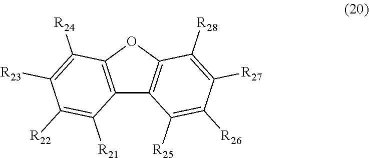
wherein in the formula (20),
one of R₂₁ to R₂₈ is a single bond bonding with Li or L₁₂;
R₂₁ to R₂₈ which are not single bonds bonding with L₁₁ or L₁₂ are independently
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃)
—O—(R₉₀₄)
—S—(R₉₀₅)
—N(R₉₀₆)(R₉₀₇)
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are as defined in the formula (11); and
adjacent two or more of R₂₁ to R₂₈ which are not single bonds bonding with L₁₁ or L₁₂ do not form a ring by bonding with each other;
Ar₁₁ₐ or Ar₁₂ₐ that is not a monovalent group represented by the formula (20) is
a substituted or unsubstituted aryl group including 6 to 50 ring carbon atoms, or
a substituted or unsubstituted monovalent heterocyclic group including 5 to 50 ring atoms, other than the monovalent group represented by the formula (20).

64. The organic electroluminescence device according to claim 63, wherein the compound represented by the formula (12) is the compound represented by the following formula (12-1): 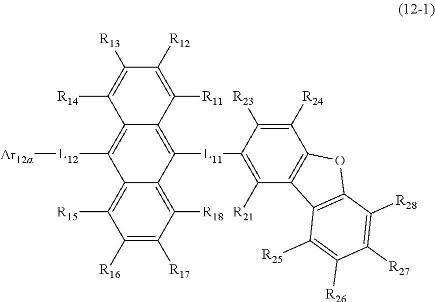
wherein in the formula (12-1), R₁₁ to R₁₈, L₁₁ and L₁₂ are as defined in the formula (11);
Ar₁₂ₐ is a substituted or unsubstituted aryl group including 6 to 50 ring carbon atoms, or
a substituted or unsubstituted monovalent heterocyclic group including 5 to 50 ring atoms, other than the monovalent group represented by the formula (20);
R₂₁ and R₂₃ to R₂₈ are independently a hydrogen atom,
a substituted or unsubstituted alkyl group including 1 to 50 carbon atoms,
a substituted or unsubstituted alkenyl group including 2 to 50 carbon atoms
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃)
—O—(R₉₀₄)
—S—(R₉₀₅)
—N(R₉₀₆)(R₉₀₇)
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms; and
R₉₀₁ to R₉₀₇ are as defined in the formula (11).

65. The organic electroluminescence device according to claim 62, wherein the compound represented by the formula (11) is the compound represented by the following formula (13): 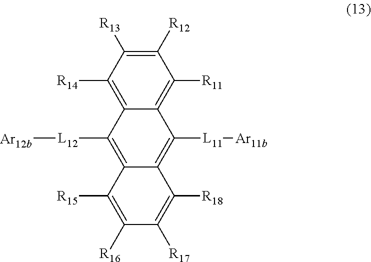
wherein in the formula (13), R₁₁ to R₁₈, Li and L₁₂ are as defined in the formula (11);
Ar_{11b} and Ar_{12b} are independently a substituted or unsubstituted aryl group including 6 to 50 ring carbon atoms, that is constituted only with a benzene ring.

66. The organic electroluminescence device according to claim 65, wherein the compound represented by the formula (13) is the compound represented by the following formula (13-1): 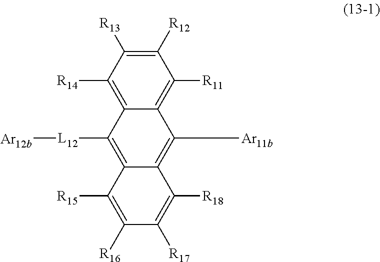
wherein in the formula (13-1), R₁₁ to R₁₈ and L₁₂ are as defined in the formula (11); and
Ar_{11b} and Ar_{12b} are independently a substituted or unsubstituted aryl group including 6 to 50 ring carbon atoms, that are constituted only with a benzene ring.

67. The organic electroluminescence device according to claim 65, wherein
Ar_{11b} and Ar_{12b} are independently
a substituted or unsubstituted phenyl group,
a substituted or unsubstituted naphthyl group,
a substituted or unsubstituted biphenylyl group,
a substituted or unsubstituted terphenylyl group,
a substituted or unsubstituted anthryl group, or
a substituted or unsubstituted phenanthryl group.

68. The organic electroluminescence device according to claim 61 wherein
one or both of the first electron-transporting layer and the second electron-transporting layer further comprises one or two or more kinds selected from the group consisting of an alkali metal, an alkaline earth metal, a rare earth metal, an alkali metal oxide, an alkali metal halide, an alkaline earth metal oxide, an alkaline earth metal halide, a rare earth metal oxide, a rare earth metal halide, an organic complex containing an alkali metal, an organic complex containing an alkaline earth metal, and an organic complex containing a rare earth metal.

69. The organic electroluminescence device according to claim 60, comprising a hole-transporting layer disposed between the anode and the emitting layer.

70. An electronic apparatus comprising the organic electroluminescence device according to claim 59.

71. The compound according to claim 41, wherein the compound represented by the formula (A1) has at least one deuterium atom.

72. The compound according to claim 41, wherein the compound represented by the formula (A1) is selected from the group consisting of: 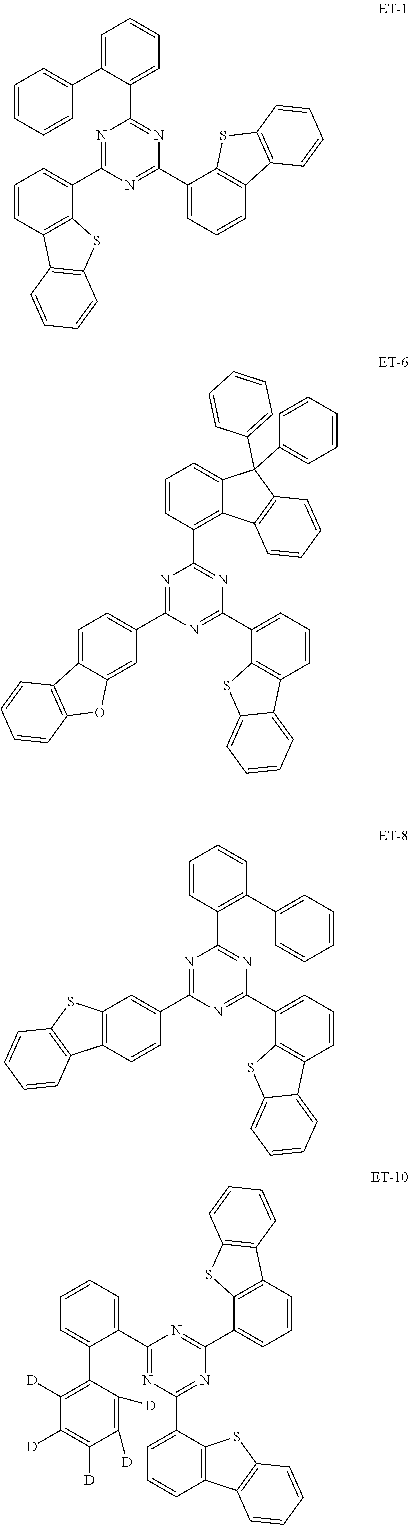 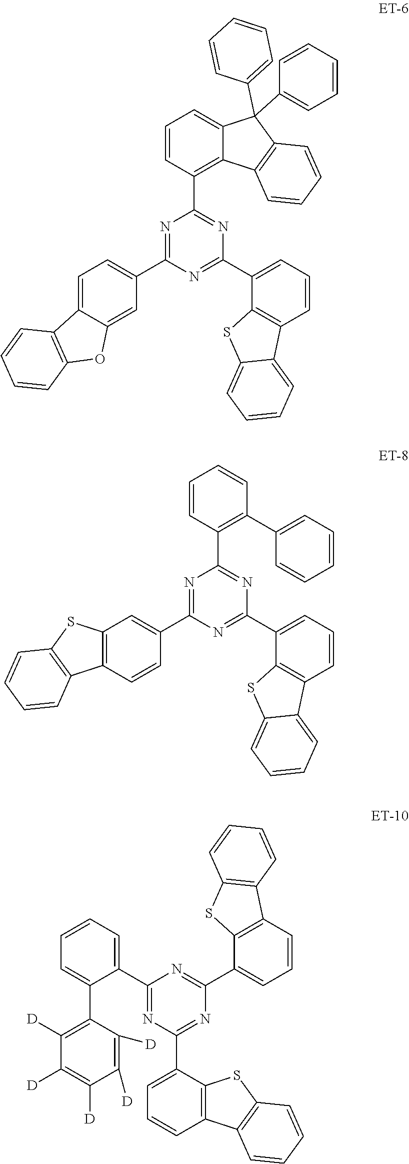 [SMILES: c1ccc(C2(c3ccccc3)c3ccccc3-c3c(-c4nc(-c5ccc6c(c5)oc5ccccc56)nc(-c5cccc6c5sc5ccccc56)n4)cccc32)cc1 ; c1ccc(-c2ccccc2-c2nc(-c3ccc4c(c3)sc3ccccc34)nc(-c3cccc4c3sc3ccccc34)n2)cc1 ; [2H]c1c([2H])c([2H])c(-c2ccccc2-c2nc(-c3cccc4c3sc3ccccc34)nc(-c3cccc4c3sc3ccccc34)n2)c([2H])c1[2H]] 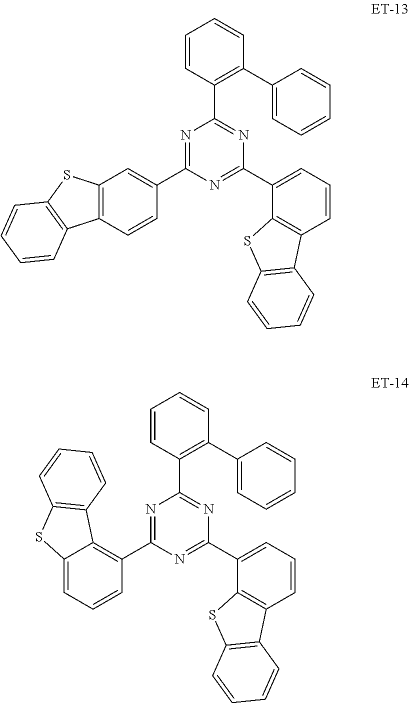 [SMILES: c1ccc(-c2ccccc2-c2nc(-c3ccc4c(c3)sc3ccccc34)nc(-c3cccc4c3sc3ccccc34)n2)cc1 ; c1ccc(-c2ccccc2-c2nc(-c3cccc4c3sc3ccccc34)nc(-c3cccc4sc5ccccc5c34)n2)cc1]
